(19) United States
(12) Patent Application Publication
(10) Pub. No.: US 2024/0254179 A1
(43) Pub. Date: Aug. 1, 2024

(54) Targeting Nuclear Speckles and DNA Speckle Association

(71) Applicant: THE TRUSTEES OF THE UNIVERSITY OF PENNSYLVANIA, Philadelphia, PA (US)

(72) Inventors: Katherine Alexander, Philadelphia, PA (US); Shelley Berger, Philadelphia, PA (US); Celeste Simon, Philadelphia, PA (US)

(21) Appl. No.: 18/418,058

(22) Filed: Jan. 19, 2024

Related U.S. Application Data
(60) Provisional application No. 63/439,914, filed on Jan. 19, 2023.

(51) Int. Cl. C07K 14/47; A61K 38/00; C07K 14/16; C12N 15/113; C12Q 1/6886

(52) U.S. Cl. CPC C07K 14/4703; C07K 14/163; C12N 15/113; C12Q 1/6886; A61K 38/00; C07K 2319/10

(57) ABSTRACT

The present invention provides polypeptides, compositions, and methods useful for the inhibition of transcription factor/DNA-speckle association and for manipulation of nuclear speckle content. Also included are methods of treating speckle related cancers in subjects in need thereof.

### CROSS-REFERENCE TO RELATED APPLICATIONS

The present application is entitled to priority under 35 U.S.C. § 119(e) to U.S. Provisional Patent Application No. 63/439,914, filed Jan. 19, 2023 which is incorporated by reference in its entirety herein.

### STATEMENT REGARDING FEDERALLY SPONSORED RESEARCH AND DEVELOPMENT

This invention was made with government support under CA078831 and CA220483 awarded by the National Institutes of Health. The government has certain rights in the invention.

### SEQUENCE LISTING

The instant application contains a Sequence Listing which has been submitted in XML format via Patent Center and is hereby incorporated by reference in its entirety. Said XML copy, created on Jan. 18, 2024, is named “046483-7359US1-Sequence-listing.xml” and is 1,615,092 bytes in size.

### BACKGROUND OF THE INVENTION

Transcription factors are key regulators of gene expression that are critical for regulating processes including development and generation of induced pluripotent stem cells. Likewise, dysregulation of transcription factor function can lead to diseases such as cancer. Many transcription factors are capable of driving different cell phenotypes and developmental outcomes depending on the cellular environment. For example, p53 activation can result in the induction of either cell death or cell survival pathways. While many tools are under development to activate or repress transcription factors, methods to toggle functional outcomes of transcription factors from one pathway to another are lacking. Shifting the type of response elicited by transcription factors is particularly impactful in cancer contexts, where transcription factor pathways are co-opted to promote cancer cell growth, invasion, and metastasis.

Nuclear speckles are nuclear structures which contain a myriad of factors involved in RNA production, and have been identified as a distinct regulatory niche in various gene expression pathways. As such, there is a need in the art for therapeutic options and prognostic indicators for transcription factor-related diseases and disorders that target or involve nuclear speckles or transcription-factor-driven DNA-speckle association. The current invention addresses this need.

### SUMMARY OF THE INVENTION

As described herein, the present invention provides polypeptides, compositions, and methods useful for the inhibition of transcription factor/DNA-speckle association and for manipulation of nuclear speckle content. Also included are methods of treating speckle related cancers in subjects in need thereof.

In one aspect, the disclosure provides a polypeptide inhibitor of transcription factor/DNA-speckle association comprising a first polypeptide domain, a second polypeptide domain, and a third polypeptide domain, wherein:
a. the first polypeptide domain comprises a cell penetrating peptide;
b. the second polypeptide domain comprises a linker region; and
c. the third polypeptide domain comprises a DNA-speckle targeting motif.

In some embodiments, the cell penetrating peptide is selected from the group consisting of an HIV TAT peptide, a penetratin peptide, an R8 peptide, a transportan peptide, a cyclic R8 peptide, a cyclic TAT peptide, an HA-TAT peptide, and an xentry peptide.

In some embodiments, the cell penetrating peptide is an HIV TAT peptide.

In some embodiments, the HIV TAT peptide comprise an amino acid sequence of GRKKRRQRRRPQ (SEQ ID NO: 2603).

In some embodiments, the linker region comprises an amino acid sequence of GGSGGGSG (SEQ ID NO: 2604).

In some embodiments, the DNA-speckle targeting motif comprises a polypeptide sequence which is at least 62 amino acids.

In some embodiments, the polypeptide sequence comprises the pattern X1(30)-X2-P-X1(30), wherein
a. X1 is any amino acid; and
b. X2 is T, S, E, or D.

In some embodiments, the polypeptide sequence does not comprise four or more consecutive proline residues.

In some embodiments, the polypeptide sequence contains proline residues in a minimum of three of positions 16, 21, 36, 41, or 46.

In some embodiments, the polypeptide sequence comprises at least five negative or phosphorylatable amino acids.

In some embodiments, the negative or phosphorylatable amino acids are selected from the group consisting of D, E, T, and S.

In some embodiments, the polypeptide sequence comprises at least five small or hydrophobic amino acids.

In some embodiments, the small or hydrophobic amino acids are selected from the group consisting of A, M, V, F, L, and I.

In some embodiments, the polypeptide sequence comprises fewer than fifteen positively charged amino acids.

In some embodiments, the positively charged amino acids are selected from the group consisting of R, H, and K.

In some embodiments, the DNA-speckle targeting motif comprises an amino acid sequence set forth in any one of SEQ ID Nos: 1-2602.

In some embodiments, the transcription factor is p53.

In some embodiments, the transcription factor is HIF2A.

In another aspect, the current disclosure provides a pharmaceutical composition comprising at least one polypeptide inhibitors of transcription factor/DNA-speckle association of any one of the above embodiments or aspects or any aspect or embodiment disclosed herein and a pharmaceutically acceptable diluent or excipient.

In another aspect, the current disclosure provides a method for inhibiting transcription factor/DNA-speckle association in a cell, comprising contacting the cell with an effective amount of an inhibitor of transcription factor/DNA-speckle association, wherein the inhibitor is the polypeptide of any one of the above aspects or embodiments or any aspect or embodiment disclosed herein.

In another aspect, the current disclosure provides a method for inhibiting transcription factor/DNA-speckle association in a cell, comprising contacting the cell with an effective amount of an inhibitor of transcription factor/DNA-speckle association, wherein the inhibitor is a small molecule.

In another aspect, the current disclosure provides a method for inhibiting transcription factor/DNA-speckle association in a cell, comprising contacting the cell with an effective amount of an inhibitor of transcription factor/DNA-speckle association, wherein the inhibitor is a combination of a small molecule and the polypeptide of any one of the above aspects or embodiments or any aspect or embodiment disclosed herein.

In another aspect, the current disclosure provides a method of treating a DNA-speckle related cancer in a subject in need thereof, comprising administering to the subject an effective amount of the pharmaceutical composition of embodiment 19, thereby treating the cancer.

In some embodiments, the cancer is clear cell renal cell carcinoma (ccRCC).

In some embodiments, the cancer is selected from the group consisting of breast cancer, cervical squamous cell carcinoma, endocervical adenocarcinoma, colon adenocarcinoma, rectum adenocarcinoma, glioblastoma, head and neck squamous cell carcinoma, kidney renal papillary cell carcinoma, glioma, liver hepatocellular carcinoma, lung squamous cell carcinoma, lung adenocarcinoma, ovarian cancer, pheochromocytoma, paraganglioma, prostate adenocarcinoma, sarcoma, skin cutaneous melanoma, stomach adenocarcinoma, tenosynovial giant cell tumor, and thymoma.

In another aspect, the current disclosure provides a method of treating a DNA-speckle related cancer in a subject in need thereof, comprising administering to the subject an effective amount of the polypeptide of any one of embodiments 1-18, thereby treating the cancer.

In some embodiments, the cancer is clear cell renal cell carcinoma (ccRCC).

In some embodiments, the cancer is selected from the group consisting of breast cancer, cervical squamous cell carcinoma, endocervical adenocarcinoma, colon adenocarcinoma, rectum adenocarcinoma, glioblastoma, head and neck squamous cell carcinoma, kidney renal papillary cell carcinoma, glioma, liver hepatocellular carcinoma, lung squamous cell carcinoma, lung adenocarcinoma, ovarian cancer, pheochromocytoma, paraganglioma, prostate adenocarcinoma, sarcoma, skin cutaneous melanoma, stomach adenocarcinoma, tenosynovial giant cell tumor, and thymoma.

In another aspect, the current disclosure provides a method of generating peptide inhibitors of DNA speckle association, the method comprising:
a. screening a library of protein sequences for those comprising a DNA-speckle targeting motif comprising:
   i. at least 62 contiguous amino acids;
   ii. comprising the pattern X1(30)-X2-P-X1(30), wherein
   iii. X1 is any amino acid; and
   iv. X2 is T, S, E, or D;
   v. does not comprise four or more consecutive proline residues;
   vi. contains proline residues in a minimum of three of positions 16, 21, 36, 41, or 46;
   vii. comprises at least five negative or phosphorylatable amino acids selected from the group consisting of D, E, T, and S;
   viii. comprises at least five small or hydrophobic amino acids selected from the group consisting of A, M, V, F, L, and I; and
   ix. comprises fewer than fifteen positively charged amino acids selected from the group consisting of R, H, and K;
b. identifying proteins comprising said motif sequence; and
c. generating peptides comprising said motif sequence.

In some embodiments, generating the peptide inhibitor further comprises adding a cell-permeability sequence to the DNA-speckle targeting motif sequence.

In some embodiments, the cell penetrating peptide is selected from the group consisting of an HIV TAT peptide, a penetratin peptide, an R8 peptide, a transportan peptide, a cyclic R8 peptide, a cyclic TAT peptide, an HA-TAT peptide, and an xentry peptide.

In some embodiments, the cell penetrating peptide is an HIV TAT peptide.

In some embodiments, the HIV TAT peptide comprise an amino acid sequence of GRKKRRQRRRPQ (SEQ ID NO: 2603).

In some embodiments, generating the peptide inhibitor further comprises adding a linker sequence between the cell-permeability sequence and the DNA-speckle targeting motif sequence.

In some embodiments, the linker region comprises an amino acid sequence of GGSGGGSG (SEQ ID NO: 2604).

In another aspect, the current disclosure provides a method of screening a tumor tissue to determine speckle signature score, comprising:
a. obtaining a specimen of tumor tissue;
b. isolating and purifying RNA from the specimen;
c. performing RNA-seq using the RNA to determine relative gene expression levels of Speckle signature genes;
d. determining the Z-score of each speckle signature gene;
e. for each speckle Signature I gene, divide its Z-score by the number of speckle protein genes in speckle Signature I, then take the sum of all these values for Signature I speckle protein genes;
f. for each speckle Signature II gene, divide its Z-score by the number of speckle protein genes in speckle Signature II, then take the sum of all these values for Signature II speckle protein genes; and
g. take the log(2) of the ratio of the result from step e to the result from step f thereby determining the speckle signature score of the specimen;
wherein, samples with high positive values are strongly Signature I and samples with low negative values are strongly Signature II.

In some embodiments, the speckle signature comprises the genes FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, and EPC2.

In some embodiments, the genes comprising speckle Signature I are selected from the group consisting of VAX2, JDP2, PLEKHN1, HDAC5, C11ORF49, SLC4A2, STYXL1, TMEM179B, TAB1, ZNF446, TBXA2R, UNC45A, PCBP1, PHLDB3, KTI12, AKAP17A, PRCC, ZNF821, SPINDOC, HSF4, DEXI, HEXIM2, EHMT2, VPS72, DDX39A, KIF22, DPCD, LHPP, CD2BP2, CDK11B, GTF2H4, DGKZ, SARNP, ALYREF, SLC2A4RG, TEPSIN, AKAP8L, PPIE, STK19, FIBP, C60RF226, H2AFX, EGFL8, PSMD13, CACTIN, EXOSC7, C120RF57, THAP4, TMEM259, THOC6, AP5Z1, PQBP1, RBM10, C1ORF35, C19ORF24, SART1, CDC34, FASTK, POMP, PRPF6, PRPF19, BRK1, UFC1, SNRPA1, ZCCHC17, SNRPB2, PCP2, SSH3, SETD1A, WDR90, THEM6, U2AF2, RBM14, MAST3, LIMK1, SF3B4, DDX39B, RTEL1, ZNF165, MAPK12, PSMD8, CDK5RAP1, PDZK1IP1, SETD4, CHTOP, CDK11A, SRSF4, TBX19, RTN2, CCDC32, CYSRT1, IQCK, MPP1, MAMSTR, ILRUN, DBNDD1, EPHB6, TCF15, C60RF52, CYGB, CCDC85C, PHYHD1, ITPKC, CDC25C, RMI2, SNRNP40, HISTIHIE, ZC3H18, SON, RBM27, TCF12, BCLAF1, ERBIN, SETD2, TCP1 IL2, EPC2, TRIP12, YLPM1, LMTK2, GPATCH8, DDX46, PRPF4B, TAB3, EPG5, RSBN1L, SF3B1, PUS7L, KCTD20, RBM26, BAZ2A, RBM41, RREB1, ZNF621, FAM160B1, CDK13, SDE2, DHX15, PRPF40A, CHIC1, SREK1, LIN52, BARD1, ZNF441, GNAQ, THRAP3, HBP1, SMC5, PPP4R3B, RBBP6, TTC26, COG6, ZC3H14, UBE3B, MRTFB, YTHDF3, UBE4A, CBLL1, API5, CMTR2, TBC1D12, WRN, KIAA1328, TMEM209, ZCCHC4, MAPK14, ZNF160, SLU7, ERCC8, FOXJ3, PCLO, RSRC1, ZC3H11A, BMP2K, RALGAPB, FBXL4, RTL6, RCAN3, FBXO34, ZBTB8A, CWF19L2, SRRM2, HELQ, FYTTD1, PPIG, ANKRD44, SOCS6, S100PBP, ZNF304, ZNF543, RBM25, EFCAB13, CPD, ARMCX5, POLI, ZNF551, MAML3, POLR3B, SFMBT2, DDX17, RNF169, KAT6A, DDX42, GPATCH2, CBFA2T2, E2F3, ZNF169, TAF5L, KIAA0100, PRKAA1, LHX4, RSRC2, CSRNP2, NCBP3, NCAPG2, SF3A1, DENND1B, BRD2, PNISR, E2F7, LRRC8B, PACSIN2, PNN, KIAA0556, SAP130, CPSF6, MAP3K7, TADA2A, HP1BP3, ZNF217, BRD1, SRRM1, SRSF11, GLYR1, FAM227B, AAGAB, PLRG1, FCHSD2, MECOM, TMEM56, CDYL, ELOA, STK17A, RIOK1, ARHGAP42, R3HCC1L, COPS4, BORCS7, THOC1, CIR1, PYROXD1, ARHGAP18, NSL1, WTAP, ZNHIT6, BCAS2, HAUS6, MORF4L1, SMC4, MBD4, PRPF18, CWC22, UBAP2L, SMURF2, KDM6B, PRKAA2, LIFR, RBM8A, SNURF, DAZAP2, FAM120C, WDR17, ZDHHC15, GTF2H2C, SRGAP1, ZSWIM5, RAF1, ZNF286B, ZNF528, ZNF572, ZNF527, XYLB, FNBP4, PRPF4, SIPA1L3, ZNF382, RFXAP, RBM39, CWC25, ZIM2, ANXA9, MFSD11, BPNT1, GPN3, MAPT, PPP1R16B, ZNF250, RAD52, ZNF786, GNB5, MNS1, TARBP1, RBM6, PRKN, ZCWPW2, MAMDC2, IPCEF1, NFATC4, LPAR1, VXN, FAM107A, IL16, USP22, RNF112, CRY2, PLAGI, IQUB, PPP1R8, BNIP3L or any combination thereof.

In some embodiments, the genes comprising speckle Signature II are selected from the group consisting of SON, RBM27, TCF12, BCLAF1, ERBIN, SETD2, TCP11L2, EPC2, TRIP12, YLPM1, LMTK2, GPATCH8, DDX46, PRPF4B, TAB3, EPG5, RSBN1L, SF3B1, PUS7L, KCTD20, RBM26, BAZ2A, RBM41, RREB1, ZNF621, FAM160B1, CDK13, SDE2, DHX15, PRPF40A, CHIC1, SREK1, LIN52, BARD1, ZNF441, GNAQ, THRAP3, HBP1, SMC5, PPP4R3B, RBBP6, TTC26, COG6, ZC3H14, UBE3B, MRTFB, YTHDF3, UBE4A, CBLL1, API5, CMTR2, TBC1D12, WRN, KIAA1328, TMEM209, ZCCHC4, MAPK14, ZNF160, SLU7, ERCC8, FOXJ3, PCLO, RSRC1, ZC3H11A, BMP2K, RALGAPB, FBXL4, RTL6, RCAN3, FBXO34, ZBTB8A, CWF19L2, SRRM2, HELQ, FYTTD1, PPIG, ANKRD44, SOCS6, S100PBP, ZNF304, ZNF543, RBM25, EFCAB13, CPD, ARMCX5, POLI, ZNF551, MAML3, POLR3B, SFMBT2, DDX17, RNF169, KAT6A, DDX42, GPATCH2, CBFA2T2, E2F3, ZNF169, TAF5L, KIAA0100, PRKAA1, LHX4, RSRC2, CSRNP2, NCBP3, NCAPG2, SF3A1, DENND1B, BRD2, PNISR, E2F7, LRRC8B, PACSIN2, PNN, KIAA0556, SAP130, CPSF6, MAP3K7, TADA2A, HP1BP3, ZNF217, BRD1, SRRM1, SRSF11, GLYR1, FAM227B, AAGAB, PLRG1, FCHSD2, MECOM, TMEM56, CDYL, ELOA, STK17A, RIOK1, ARHGAP42, R3HCC1L, COPS4, BORCS7, THOC1, CIR1, PYROXD1, ARHGAP18, NSL1, WTAP, ZNHIT6, BCAS2, HAUS6, MORF4L1, SMC4, MBD4, PRPF18, CWC22, UBAP2L, SMURF2, KDM6B, PRKAA2, LIFR, RBM8A, SNURF, DAZAP2, FAM120C, WDR17, ZDHHC15, GTF2H2C, SRGAP1, ZSWIM5, RAF1, ZNF286B, ZNF528, ZNF572, ZNF527, XYLB, FNBP4, PRPF4, SIPA1L3, ZNF382, RFXAP, RBM39, CWC25, ZIM2, ANXA9, MFSD11, BPNT1, GPN3, MAPT, PPP1R16B, ZNF250, RAD52, ZNF786, GNB5, MNS1, TARBP1, RBM6, PRKN, ZCWPW2, MAMDC2, IPCEF1, NFATC4, LPAR1, VXN, FAM107A, IL16, USP22, RNF112, CRY2, PLAGI, IQUB, PPP1R8, BNIP3L, VAX2, JDP2, PLEKHN1, HDAC5, C11ORF49, SLC4A2, STYXL1, TMEM179B, TAB1, ZNF446, TBXA2R, UNC45A, PCBP1, PHLDB3, KTI12, AKAP17A, PRCC, ZNF821, SPINDOC, HSF4, DEXI, HEXIM2, EHMT2, VPS72, DDX39A, KIF22, DPCD, LHPP, CD2BP2, CDK11B, GTF2H4, DGKZ, SARNP, ALYREF, SLC2A4RG, TEPSIN, AKAP8L, PPIE, STK19, FIBP, C60RF226, H2AFX, EGFL8, PSMD13, CACTIN, EXOSC7, C120RF57, THAP4, TMEM259, THOC6, AP5Z1, PQBP1, RBM10, C1ORF35, C19ORF24, SART1, CDC34, FASTK, POMP, PRPF6, PRPF19, BRK1, UFC1, SNRPA1, ZCCHC17, SNRPB2, PCP2, SSH3, SETD1A, WDR90, THEM6, U2AF2, RBM14, MAST3, LIMK1, SF3B4, DDX39B, RTEL1, ZNF165, MAPK12, PSMD8, CDK5RAP1, PDZK1IP1, SETD4, CHTOP, CDK11A, SRSF4, TBX19, RTN2, CCDC32, CYSRT1, IQCK, MPP1, MAMSTR, ILRUN, DBNDD1, EPHB6, TCF15, C60RF52, CYGB, CCDC85C, PHYHD1, ITPKC, CDC25C, RMI2, SNRNP40, HIST1H1E, ZC3H18.

In another aspect, the current disclosure provides a method of treating a Speckle signature associated cancer in a subject in need thereof, comprising:
a. obtaining a specimen of tumor tissue;
b. isolating and purifying RNA from the specimen;
c. performing RNA-seq using the RNA to determine the speckle signature of the tumor tissue; and
d. administering an effective amount of an inhibitor of expression for at least one speckle signature gene, thereby treating the cancer; In some embodiments, the method further comprises determining the nuclear localization profile of at least one speckle signature gene.

In some embodiments, a radial nuclear localization profile correlates with worse prognosis.

In some embodiments, the at least one inhibited speckle gene is associated with speckle Signature I.

In some embodiments, the inhibition of at least one gene associated with Speckle Signature I shifts the Speckle signature of the tumor tissue to Speckle Signature II.

In some embodiments, the at least one inhibited Speckle gene is associated with Speckle Signature II.

In some embodiments, the inhibition of at least one gene associated with Speckle Signature II shifts the Speckle signature of the tumor tissue to Speckle Signature I.

In some embodiments, shifting the Speckle signature of the tumor tissue improves prognosis.

In some embodiments, the cancer is selected from the group consisting of clear cell renal cell carcinoma, KMT2D wild type melanoma, TTN wild type lung adenocarcinoma, BRAF wild type thyroid cancer, and PIK3R1 mutant endometrial cancer.

In some embodiments, the inhibitor of Speckle signature gene expression is selected from the group consisting of an inhibitory RNA, a small molecule, a PROTAC, a CRISPR/Cas9 system, and any combination thereof.

In some embodiments, the inhibitory RNA is selected from the group consisting of an siRNA, and an shRNA or any combination thereof.

In some embodiments, the Speckle signature gene is SART1.

In some embodiments, the speckle signature gene is HBP1.

In some embodiments, the speckle signature gene is COPS4

In some embodiments, the speckle signature is determined by immunofluorescence of FFPE tumor samples.

In some embodiments, the speckle signature is determined by RNA or protein analysis of a subset of speckle protein genes comprising FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, EPC2, or any combination thereof.

In another aspect, the current disclosure provides a method of determining the prognosis of a speckle-related cancer in a subject in need thereof, comprising:
a. obtaining a specimen of cancer tissue;
b. preparing the tissue specimen such that nuclear localization of at least one speckle-related protein can be visualized and quantified; and
c. determining the nuclear localization profile of at least one speckle-related protein in the tissue, thereby indicating the severity of the speckle-related cancer;
wherein radial positioning speckle-related protein expression indicates a worse prognosis.

In some embodiments, the at least one speckle-related protein is selected from the group consisting of FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, EPC2, or any combination thereof.

In some embodiments, the at least one speckle-related protein is SON.

In some embodiments, the visualization and quantification of the speckle protein localization comprises immunofluorescence microscopy.

In some embodiments, the cancer is selected from the group consisting of clear cell renal cell carcinoma, KMT2D wild type melanoma, TTN wild type lung adenocarcinoma, BRAF wild type thyroid cancer, and PIK3R1 mutant endometrial cancer.

In another aspect, the current disclosure provides a method of treating a speckle-related cancer in a subject in need thereof, comprising:
a. performing RNA-seq using RNA purified from a tumor specimen from the subject to determine the speckle signature of the tumor tissue; and
b. administering an effective amount of an anticancer therapeutic, thereby treating the cancer;

wherein, the sensitivity of the tumor to the anticancer therapeutic correlates with the speckle signature of the tumor tissue.

In some embodiments, the method further comprises determining the nuclear localization profile nuclear speckles.

In some embodiments, the speckle signature is associated with speckle signature I.

In some embodiments, the speckle signature is associated with speckle Signature II.

In some embodiments, choosing a speckle signature correlated treatment strategy improves treatment prognosis.

In some embodiments, the cancer is selected from the group consisting of clear cell renal cell carcinoma, neuroblastoma, KMT2D wild type melanoma, TTN wild type lung adenocarcinoma, BRAF wild type thyroid cancer, and PIK3R1 mutant endometrial cancer.

In some embodiments, the cancer is clear cell renal cell carcinoma.

In some embodiments, the anticancer therapeutic is selected from the group consisting of an a biologic, a small molecule, an immunotherapy, and any combination thereof.

In some embodiments, immunotherapy is an immune checkpoint inhibitor.

In some embodiments, the immune checkpoint inhibitor is an inhibitor of PD-1.

In some embodiments, the PD-1 inhibitor is nivolumab.

In some embodiments, the anticancer therapeutic is an inhibitor of HIF-2α.

In some embodiments, the inhibitor of HIF-2α is PT2399.

In some embodiments, the speckle signature is determined by the nuclear localization profile of nuclear speckles.

In some embodiments, the nuclear localization profile is determined by immunofluorescence of FFPE tumor samples.

In some embodiments, the speckle signature is determined by RNA or protein analysis of a subset of speckle protein genes comprising FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, EPC2, or any combination thereof.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 illustrates the mapping of the critical amino acids for p53-mediated speckle association of p53 target gene, p21. Scanning point mutations spanning the p53 second transactivation domain and proline rich domain identified critical amino acids for p53-mediated speckle association of p53 target gene, p21. Distance of the p21 genomic locus was measured by immunoDNA-FISH upon wild type (WT) or mutant induced expression in Saos2 cells using doxycycline to induce p53 expression for 3 hours. The D57A mutation improved p53-mediated speckle association of p21 (p<0.01). The T81A mutation compromised p53-mediated speckle association of p21 (p<0.0005).

FIG. 2 is a diagram of the p53 proline-rich domain amino acid sequence and surrounding regions. The deletion that compromised p53-mediated speckle association of p21 in recently published studies is underlined. Specific amino acid locations within p53 are indicated above the sequence. Hydrophobic amino acids are highlighted in red; acidic amino acids are highlighted in dark blue; phosphorylatable amino acids are highlighted in light blue. The D57 and T81 amino acids that affect p53-mediated speckle association (see FIG. 1) are in white text.

FIG. 3 illustrates that the charged state of p53 amino acid positions 55, 57, and 81 dictates p53 ability to mediate speckle association of target gene, p21. Distance of the p21 genomic locus was measured by immunoDNA-FISH under p53 null conditions or upon wild type (WT) or mutant-induced expression in Saos2 cells using doxycycline to induce p53 expression for 3 hours. Speckles were stained with the speckle-marker protein, SON, and the distance of the p21 locus and the nearest speckle was measured. Mutation of T55 to unphosphorylatable A did not alter speckle association status, but mutation of T55 to phosphomimetic D compromised speckle association. Eliminating the negative charge of D57 improved speckle association. Unphosphorylatable and phosphomimetic mutations of T81 had the opposite effect as compared to T55 mutations: T81A compromised speckle association (as in FIG. 1), while the T81D mutation was competent at speckle association. These results indicate that the distribution of charge within the speckle targeting motif is critical for speckle targeting capacities of p53.

FIG. 4. illustrates the treatment of HeLa cells with the hypoxia mimic, CoCl2, results in increased speckle association of the HIF2A target gene CCND1. Speckle association was measured by immunoRNA-FISH, with sites of transcription defined as the overlap between intronic and exonic probe set spots, in untreated HeLa cells and in HeLa cells treated with CoCl2 to mimic hypoxic conditions.

FIGS. 5A-5B illustrates on target activity of HIF2A-inhibitor, PT2399. (FIG. 5A) RNA-seq in DMSO control and during a PT2399 time course reveals gene regulated by HIF as the top decreasing genes (GO analysis not shown), and shows that the majority of genes are decreasing with HIF2A inhibition, consistent with HIF2A being a transcriptional activator. (FIG. 5B) ChIP-seq in DMSO control or PT2399 treatment reveals a loss of HIF2A-specific binding upon PT2399 inhibition, confirming on target activity of PT2399 in 786O cells. The top enriched transcription factor binding motif in the DMSO control was HIF2A.

FIG. 6 illustrates that SON TSA-seq reveals regulation of speckle association by HIF2A. Speckle association as measured by SON TSA-seq decreased at the HIF2A-responsive gene DDIT4 upon HIF2A inhibition (left). HIF2A binding sites are shown as lines above genome-browser tracks. HIF2A alters speckle association of its responsive genes to varying extents (right). In total, 175 of 697 HIF2A responsive genes were found to have HIF2A-regulated changes in SON TSA-seq signal.

FIG. 7. Local alignment between p53 and HIF2A identified the strongest region of homology to the p53 proline rich domain, identifying it as a speckle targeting motif present in both proteins. Full length p53 and full length HIF2A peptide sequences were aligned using a local similarity pairwise alignment tool, EMBOSS Matcher, which revealed 37.9% identity, 55.2% similarity between p53 amino acids 62-90 (amino acids 57-102 are shown in displayed alignment) and HIF2A amino acids 450-478 (HIF2A_1; amino acids 445-490 are shown in displayed alignment). After definition of the speckle targeting motif, a second speckle targeting motif was identified in HIF2A (HIF2A_2; amino acids 766-811 are shown in displayed alignment).

FIG. 8 is a diagram of the network of protein-protein interactions among proteins that contain speckle targeting motif (from STRINGDB). Network edges represent physical protein interactions. The network is significantly more interconnected than expected by random chance (STRING-DB; p<1⁻¹⁶). The network is enriched for “Regulation of transcription by RNA polymerase II” (top Biological Process, GO, FDR<1⁻²⁸; highlighted in red), “DNA-binding transcription factor activity, RNA polymerase II-specific” (top Molecular Function, GO, FDR<1⁻²⁷), and “Nuclear chromatin” (top Cellular Compartment, GO, FDR<1⁻¹⁹).

FIG. 9 is a diagram illustrating that nuclear proteins (red) among proteins that contain speckle targeting motif Same protein network as in FIG. 8 with the nuclear compartment proteins highlighted in red (FDR enrichment <1⁻¹⁵) and “Developmental disorder of mental health” disease gene association in blue (FDR enrichment <0.005).

FIG. 10 is a diagram illustrating the speckle targeting domain of HOXB13 with familial prostate cancer mutations indicated with arrows.

FIG. 11 illustrates the loss of speckle association at HIF2A-responsive genes upon HIF2A inhibition with PT2399 in 786O cells. CCND1 and DDIT4 become more distal to speckles upon HIF2A inhibition (left). Under DMSO HIF2A active conditions, CCND1 and DDIT4 show the characteristic L-shaped relationship between distance to speckle and amount of nascent RNA within transcription sites (as estimated by the intensity of exonic RNA-FISH spot at the site of transcription [defined by overlap between exonic and intronic RNA-FISH spot] relative to the median intensity of smRNA-FISH exonic spot within the same cell), consistent with previous observations of p53-mediated speckle association that speckle association results in boosted RNA production. Treatment of cells with PT2399 abolishes this L-shaped distribution (right scatterplot).

FIG. 12 illustrates that the inhibition of HIF2A with PT2399 does not alter speckle association of HIF2A-responsive genes in A498 cells. ImmunoRNA-FISH was performed as in FIG. 11. However, in contrast to 786O cells, A498 cells do not display HIF2A dependent changes in gene-speckle association, and do not show the characteristic L-shaped relationship between nascent RNA within transcription sites and speckle distances.

FIG. 13 is a diagram illustrating the overlap between HIF2A-responsive genes in 786O cells and A498 cells.

FIG. 14 illustrates that expression of speckle protein genes in VHL-mutated ccRCC falls into three tissue clusters with distinct speckle protein gene expression patterns. Speckle protein genes show one of two dysfunction patterns compared to tissue matched controls. speckle Signature I patients (top cluster) show opposite speckle protein gene expression patterns compared to speckle Signature II patients (bottom cluster).

FIG. 15 illustrates that patient speckle protein gene expression signature is significantly associated with ccRCC tumor stage, metastasis status, and overall survival probability, with patients with speckle Signature I as defined in FIG. 14 enriched among patients with later stage tumors, metastatic disease, and poorer survival.

FIG. 16 illustrates that the top mutated genes in ccRCC differ in expression between patient speckle signature groups. Displayed on the left is the heatmap of Z-scores of the median expression of the top mutated genes in each sample group. Genes that were higher in tumor versus normal tended to be higher in tumors with speckle Signature I versus II. Reciprocally, genes that are lower in ccRCC versus normal tissue tend to be lower in speckle Signature I versus II. On the right is a boxplot representation of the highly mutated ccRCC gene PBRM1 showing lower expression in patients with speckle signature I. I—speckle Signature I patient group; N—matched adjacent tissue; II—speckle Signature II patient group.

FIG. 17 illustrates that speckle signature corresponds to altered patterns of HIF2A gene expression. Heatmap showing Z-scores of the median expression of HIF2A-responsive genes defined by RNA-seq of PT2399 treatment in 786O cells and A498 cells. I—speckle Signature I patient group; N—matched adjacent tissue; II—speckle Signature II patient group.

FIG. 18 illustrates that the observed patient biases between speckle Signature I and II patients of HIF2A-responsive genes is highly correlated with DNA-speckle association. Displayed on the right are the four HIF2A-responsive gene cluster from FIG. 17, showing that the signature I-biased HIF2A-responsive clusters i and iv have significantly higher speckle association than the signature II-biased HIF2A-responsive clusters ii and iii as determined from the amount of signal from SON TSA-seq genome-wide measurements of speckle association in 786O cells. Displayed on the right is a scatterplot showing the ratio of the median expression of each HIF2A-responsive gene in the Signature I to the Signature II patient group (x-axis) versus the SON TSA-seq speckle signal (y-axis). Together these data demonstrate that the speckle Signature I patient group preferentially expresses speckle-associating HIF2A-responsive genes, while the speckle Signature II patient group preferentially expresses non-speckle-associating HIF2A-responsive genes.

FIG. 19 illustrates that 786O-specific HIF2A-responsive genes tend to be higher in the speckle Signature I patient group; A498-specific HIF2A-responsive genes tend to be higher in the Signature II patient group. Groups of HIF2A-responsive genes (see FIG. 13) and their expression ratio between the two speckle signature patient groups defined as in FIG. 14.

FIG. 20 illustrates that knockdown of SART1 resulted in decreased expression of speckle-associating genes (Group 10) and increased expression of non-speckle-associated genes (Group 1) in 786O cells. Log 2 fold change is shown relative to a nontargeting siRNA control. Results are similar for the two SART1 siRNAs used, siRNA4 (left) and siRNA6 (right).

FIG. 21 illustrates that genes that decrease upon SART1 knockdown have higher levels of speckle association; genes that increase upon SART1 knockdown have lower levels of speckle association. Increasing and decreasing genes were combined between the two SART1 siRNAs, not significant genes were included only if they were not significant in each of the siRNA conditions.

FIG. 22 illustrates that knockdown of SART1 in 786O cells results in decreased expression of signature I-biased genes (Groups 6-10) and increased expression of Signature II-biased genes (Groups 1-4). These data demonstrate that SART1 knockdown is sufficient to transform 786O cells toward a speckle signature II-like expression phenotype.

FIG. 23 illustrates that genes decreasing upon SART1 knockdown have higher expression in the speckle Signature I patient group, while genes increasing upon SART1 knockdown have higher expression in the speckle Signature II patient group. These data demonstrate that SART1 knockdown is sufficient to transform 786O cells toward a speckle signature II-like expression phenotype.

FIG. 24 illustrates that knockdown of SART1 results in a global upregulation of Signature II speckle protein genes. The RNA-seq expression fold change of Signature I and Signature II speckle protein genes was examined in each SART1 knockdown (kd4 and kd6) relative to the non-targeting control (NTC). SART1 knockdown resulted in a slight overall decrease in the expression of other Signature I speckle protein genes (ttest with fold change of 0 as null hypothesis p<0.05), and a major overall increase in Signature II speckle protein genes (ttest with fold change of 0 as null hypothesis p<1e-5). These data suggest a speckle signature regulatory circuit.

FIG. 25 illustrates that knockdown of HBP1 Signature II speckle protein gene shifts A498 cells towards a signature I-like expression phenotype.

FIG. 26 illustrates that knockdown of COPS4 Signature II speckle protein gene shifts A498 cells towards a signature I-like expression phenotype.

FIG. 27 illustrates examples of two genes that have highly correlated expression with the speckle score, GADD45GIP1 (high in signature I) and LATS1 (high in signature II). Given the strong correlation with speckle score, expression of these genes may be genomic readouts of the speckle signature.

FIG. 28 illustrates that speckle Signature I is associated with poorer outcomes in KMT2D wild type melanoma.

FIG. 29 illustrates that speckle Signature II is associated with poorer outcomes in BRAF wild type thyroid cancer.

FIG. 30 illustrates that speckle Signature I is associated with poorer outcomes in PIK3R1 mutant endometrial cancer.

FIG. 31 illustrates that speckle Signature II is associated with poorer outcomes in TTN wild type lung adenocarcinomas.

FIG. 32 illustrates that mutated p53 is associated with poorer survival in speckle Signature I lung adenocarcinomas, but does not reach statistical significance in speckle Signature II lung adenocarcinomas.

FIG. 33 is a table illustrating Enriched Biological Processes from STRING-DB analysis of the speckle target motif-containing proteins.

FIG. 34 is a table illustrating Enriched Molecular Functions from STRING-DB analysis of the speckle target motif-containing proteins.

FIG. 35 is a table illustrating Enriched Cellular Components from STRING-DB analysis of the speckle target motif-containing proteins.

FIGS. 36A-36G-1 are a table illustrating speckle protein genes and their individual ability to predict patient outcomes in the kirc TCGA dataset (as per Xena browser), whether poor prognosis is associated with high or low expression of that speckle protein gene, the p-value of the correlation between gene expression and tumor pathology grade, the athology grade associated with high speckle protein gene expression, and our assessment of the degree of speckle enrichment of the speckle protein genes from the Human Protein Atlas-designated speckle resident proteins. The absence of values indicates nonsignificant p-values.

FIGS. 37A-37E are a table illustrating speckle protein genes contributing most to patient variation for 20 cancer types.

FIGS. 38A-38D is a table illustrating the number of overlapping Signature I speckle protein genes between each cancer type (top) and the p-values of the significance of the overlap (bottom).

FIG. 39 is a table illustrating the number of overlapping Signature II speckle protein genes between each cancer type (top) and the p-values of the significance of the overlap (bottom).

FIG. 40 is a diagram illustrating aspects of nuclear speckle staining positioning within the nucleus which were used in the correlation with patient prognosis and survival.

FIG. 41 illustrates Kaplan Meier plots showing survival statistics for the fraction of SON signal in each radial distribution bin. Bin 1 of 4 represents the center bin of the nucleus, with 2 of 4 being the second bin from center, 3 of 4 being the third bin from center, and 4 of 4 being the peripheral bin. Patient groups were split into the top or bottom 50% of each measurement based on the median value of all nuclei measured in that patient ccRCC sample.

FIG. 42 is a table of variables found to predict ccRCC survival.

FIG. 43 is a table and heatmap demonstrating how certain variables predictive of ccRCC survival correlate with one another.

FIG. 44 illustrates that SON localization is less central in ccRCC versus adjacent tissue.

FIG. 45 illustrates the scoring of SON nuclear staining localization.

FIG. 46 illustrates an example immunofluorescence microscopy images of SON (red) and DAPI (cyan) signal in ccRCC tumor samples with high fraction of SON at center (top) or high fraction SON at periphery (bottom). The alphanumerical code at the top right of each image indicates the sample location on the tissue microarray.

FIG. 47 illustrates violin plots showing the fraction of SON signal in the center of the nucleus (FractAtD1of4; left) and at the nuclear periphery (FractAtD4of4; right) in adjacent tissues and ccRCC samples separated by tumor grade.

FIG. 48 is a Kaplan Meier plot showing survival for the fraction of SON signal in the center of the nucleus (FractAtD1of4) for Grade 1, Grade 1/2, and Grade 2 ccRCC patients (excluding Grade 2/3 and Grade 3).

FIG. 49 is a table showing Cox proportional hazard statistics in a survival model using Age, fraction SON at center of nucleus (FractAtD1of4 for SON), and the coefficient of variation of DAPI signal at the center of the nucleus (RadialCV1or4 for DAPI) as variables. A p-value of less than 0.05 is considered to be statistically significant. This model accounting for Age, SON radial positioning, and DAPI signal variation at the center of the nucleus is highly significant by all metrics tested (statistics below table).

FIG. 50 is a table showing Kaplan Meier statistics for each nuclear imaging variable measured. A p-value of less than 0.05 is considered to be statistically significant.

FIG. 51 is a graph illustrating that speckle signature correlates with patient outcomes in neuroblastoma using RNA-seq and survival data from the TARGET 2018 neuroblastoma cohort.

FIG. 52 illustrates the relationship between speckle signature score and the fraction of SON in the nucleus center from ccRCC tumor and adjacent normal samples in split for RNA and imaging (as in schematic, left). Tx—Xenograft tumor from mice; all four are from the same individual donor, different mice. T—primary tumor. N—tumor-adjacent normal samples.

FIG. 53 illustrates speckle signature scores calculated from RNAseq data of patient-derived mouse xenograft tumors that were resistant or sensitive to PT2399 HIF-2A inhibition. Data represents 18 resistant and 19 sensitive mouse xenograft tumors derived from 9 total individuals.

FIG. 54 illustrates ccRCC signature I (left) or Signature II (right) patient overall survival Kaplan Meier plots in response to nivolumab (PD1 inhibitor) or everolimus (mTOR inhibitor). Signature I nivolumab n=97; Signature I everolimus n=52; Signature II nivolumab n=84; Signature II everolimus n=78.

FIGS. 55A-55C illustrates the correlation of speckle gene signature to disease outcomes of various cancer types. FIG. 55A is a schematic showing the generation of multi-cancer speckle signatures. Proteins residing within speckles were identified, their expression evaluated, contributions to patient variation compared (Pearson's correlations), and consistent speckle protein gene contributors to patient variation were identified. FIG. 55B illustrates heatmaps showing z-scores of speckle signature protein gene RNA expression in melanoma (SKCM), breast cancer (BRCA), and renal cell carcinoma (KIRC). Bar on left represents speckle scores. Bar above represents Signature I- (cyan) or Signature II (pink) high speckle protein genes. FIG. 55C illustrates Kaplan Meier plots separating cancer cohorts by the top and bottom 25% of speckle scores.

FIGS. 56A-56B illustrate that Signature I or II gene expression patterns correspond to differential functional pathways in many different cancer types. FIG. 55A. Example gene set enrichment plots for breast cancer (BRCA), melanoma (SKCM), and ccRCC (KIRC) for Hallmark (left) and KEGG (right) of gene expression biases between speckle Signature I and II patient groups. FIG. 56B illustrates Hallmark and KEGG gene set enrichment statistics for Signature I versus Signature II speckle patient groups. ccRCC (KIRC) is in red text.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a”, “an”, and “the” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or +10%, more preferably +5%, even more preferably +1%, and still more preferably +0.1% from the specified value, as such variations are appropriate to perform the disclosed methods.

A “biomarker” or “marker” as used herein generally refers to a nucleic acid molecule, clinical indicator, protein, or other analyte that is associated with a disease. In certain embodiments, a nucleic acid biomarker is indicative of the presence in a sample of a pathogenic organism, including but not limited to, viruses, viroids, bacteria, fungi, helminths, and protozoa. In various embodiments, a marker is differentially present in a biological sample obtained from a subject having or at risk of developing a disease (e.g., an infectious disease) relative to a reference. A marker is differentially present if the mean or median level of the biomarker present in the sample is statistically different from the level present in a reference. A reference level may be, for example, the level present in an environmental sample obtained from a clean or uncontaminated source. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing a disease (e.g., an infectious disease), for determining the prognosis of the subject, for evaluating therapeutic efficacy, or for selecting a treatment regimen.

By “agent” is meant any nucleic acid molecule, small molecule chemical compound, antibody, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

The term “co-activator” refers to a protein that binds indirectly to DNA that positively regulates gene expression.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

By “fragment” is meant a portion of a nucleic acid or polypeptide molecule. This portion contains, preferably, at least 10%, 20%, 30%, 40%, 50%, 60%, 70%, 80%, or 90% of the entire length of the reference nucleic acid molecule or polypeptide. A fragment may contain 5, 10, 15, 20, 30, 40, 50, 60, 70, 80, 90, 100 or more nucleotides or amino acids.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. In some cases, homology can also be defined as analogous subunit positions in two molecules, such as polypeptides, having biochemically similar residues (e.g. a serine and/or a threonine, as both have polar and uncharged side chains). The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Hybridization” means hydrogen bonding, which may be Watson-Crick, Hoogsteen or reversed Hoogsteen hydrogen bonding, between complementary nucleobases. For example, adenine and thymine are complementary nucleotides that pair through the formation of hydrogen bonds.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “marker profile” is meant a characterization of the signal, level, expression or expression level of two or more markers (e.g., polynucleotides).

By the term “microbe” is meant any and all organisms classed within the commonly used term “microbiology,” including but not limited to, bacteria, viruses, fungi and parasites.

By the term “microarray” is meant a collection of nucleic acid probes immobilized on a substrate. As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring. Nucleic acid molecules useful in the methods of the invention include any nucleic acid molecule that specifically binds a target nucleic acid (e.g., a nucleic acid biomarker). Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “nuclear speckle” refers to the specific type of membrane-less body or compartment within the cell nucleus. Nuclear speckle structures, which are also called interchromatin granule clusters, are sites of gene expression, including transcription, RNA splicing factor storage and modification, as well as RNA metabolism, that is marked by high enrichment of the protein SON and/or the protein SRRM2.

The term “nuclear speckle protein” refers to a protein that resides within nuclear speckles.

“Parenteral” administration of a composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By “reference” is meant a standard of comparison. As is apparent to one skilled in the art, an appropriate reference is where an element is changed in order to determine the effect of the element. In one embodiment, the level of a target nucleic acid molecule present in a sample may be compared to the level of the target nucleic acid molecule present in a clean or uncontaminated sample. For example, the level of a target nucleic acid molecule present in a sample may be compared to the level of the target nucleic acid molecule present in a corresponding healthy cell or tissue or in a diseased cell or tissue (e.g., a cell or tissue derived from a subject having a disease, disorder, or condition).

As used herein, the term “sample” includes a biologic sample such as any tissue, cell, fluid, or other material derived from an organism.

By “specifically binds” is meant a compound (e.g., nucleic acid probe or primer) that recognizes and binds a molecule (e.g., a nucleic acid biomarker), but which does not substantially recognize and bind other molecules in a sample, for example, a biological sample.

The term “speckle targeting motif” refers to a peptide sequence or collection of related peptide sequences found within proteins that are required for the DNA nuclear speckle targeting ability of the transcription factor proteins.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, and more preferably more, such as 80% or 85%, and more preferably 90%, 95%, 96%, 97%, 98%, or even 99% or more identical at the amino acid level or nucleic acid to the sequence used for comparison.

Sequence identity and homology is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In another exemplary approach, a BLOSOM substitution matrix may be used to score conservative and/or non-conservative substitutions.

By the term “substantially microbial hybridization signature” is a relative term and means a hybridization signature that indicates the presence of more microbes in a tumor sample than in a reference sample. By the term “substantially not a microbial hybridization signature” is a relative term and means a hybridization signature that indicates the presence of less microbes in a reference sample than in a tumor sample.

By “subject” is meant a mammal, including, but not limited to, a human or non-human mammal, such as a bovine, equine, canine, ovine, feline, mouse, or monkey. The term “subject” may refer to an animal, which is the object of treatment, observation, or experiment (e.g., a patient).

By “target nucleic acid molecule” is meant a polynucleotide to be analyzed. Such polynucleotide may be a sense or antisense strand of the target sequence. The term “target nucleic acid molecule” also refers to amplicons of the original target sequence. In various embodiments, the target nucleic acid molecule is one or more nucleic acid biomarkers.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “TSA-seq” or Tyramide Signal Amplification sequencing is a genetic mapping tool which estimates the mean chromosomal distances to defined nuclear structures, including nuclear speckles. TSA-seq makes use of the tyramide signal amplification staining method to generate biotin-tyramide free radicals, which are generated by peroxidases coupled to antibodies. The exponential decay in concentration of these free radicals, spreading radially from the antibody staining target, establishes a “cytological ruler,” allowing estimation of distance of chromosome loci from the staining target by measuring biotin labeling across the genome. TSA-seq can be used to determine interactions between gene loci and nuclear speckles.

By the term “tumor tissue sample” is meant any sample from a tumor in a subject including any solid and non-solid tumor in the subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to compositions and methods for manipulating nuclear speckles, DNA-speckle contacts, and inducible DNA-speckle association to shift gene expression. In other embodiments, the present invention relates to using the speckle signature defined by the inventors as a prognostic indicator to define subject subclasses whom would benefit from particular therapeutic strategies. The compositions and methods of the present invention will be applied to human therapies that involve altered gene expression programs driven by nuclear speckles or by speckle-targeting transcription factors, including, but not limited to, human cancer such as clear cell renal cell carcinoma, neuroblastoma, melanoma, thyroid cancer, endometrial cancer, lung adenocarcinoma, cancers with gain-of-function p53 mutations, and cancers with wild type p53 where p53 activation is a therapeutic strategy.

### Inhibitors of DNA-Speckle Association

In some aspects, the present invention provides polypeptides and compositions for inhibiting transcription-factor driven DNA-speckle contacts by cellular proteins such as transcription factors, co-activators, and the like. In certain embodiments, the transcription factors which mediate association with DNA-speckles are p53 and HIF2A. It is also contemplated that the polypeptides and compositions of the invention can be used to inhibit the DNA-speckle association of any transcription factor that drives DNA-speckle association through the presence of a DNA-speckle targeting motif within the transcription factor (see Tables 1 and 2 for a non-limiting list of transcription factors and their putative speckle targeting motifs). Transcription factors which possess a DNA-speckle targeting motif include, but are not limited to key players in stem cell pluripotency that are manipulated in pluripotent stem cell therapies (OCT4, KLF4, and TOX4), commonly mutated tumor suppressors (KMT2C and KMT2D), neurogenesis and neurodegeneration-related factors transcription factors (HTT, NEUROD1), factors involved in T cell functions and T cell exhaustion (NFATC4, FLIT, TOX2, and HIVEP3), and a transcription factor with point mutations within the speckle targeting motif associated with familial risk of prostate cancer (HOXB13, (Beebe-Dimmer et al., 2015; Breyer et al., 2012; Dupont et al., 2021; Ewing et al., 2012; Heise et al., 2019; Wei et al., 2021)). The polypeptides, compositions, and methods disclosed herein are immediately relevant to cancer therapies for cancers which possess gain-of-function p53 mutations and HIF2A hyperactivation (e.g. clear cell renal cell carcinoma, pheochromocytomas, retinal hemagiomas).

### Speckle Targeting Blocking Peptide Components

In some aspects, the current invention provides an inhibitor of transcription factor/DNA-speckle association that is a polypeptide comprising a first polypeptide domain, a second polypeptide domain, and a third polypeptide domain wherein the first polypeptide domain comprises a cell penetrating peptide, the second polypeptide domain comprises a linker region, and the third polypeptide domain comprises a DNA-speckle targeting motif. In some aspects, the current invention also includes a fourth polypeptide domain that comprises a nuclear localization signal.

In some embodiments, the first polypeptide domain comprises a cell penetrating peptide. Unlike many small-molecule drugs, which can diffuse into cells through the plasma membrane, proteins including the polypeptides of the invention are relatively large and hydrophilic molecules and as such are not able to pass directly through the plasma membrane. Cell-penetrating peptides or domains are typically composed of 5 to 30 amino acids and are positively charged at physiological pH and induce the endocytosis of the peptide or the protein to which it is conjugated to by a number of different mechanisms including, but not limited to direct penetration, endosomal uptake, and endocytic pathways. In some embodiments, the cell penetrating peptide is an HIV TAT peptide. In some preferred embodiments, the HIV TAT peptide has an amino acid sequence of GRKKRRQRRRPQ (SEQ ID NO: 1731). It is also contemplated that the polypeptides of the current invention can utilize any number of cell penetrating peptides known in the art including penetratin, R8, transportan, and xentry among others. In some embodiments, the polypeptides of the current invention comprise modified cell-penetrating peptides, which can include but are not limited to cyclic R8 peptides, cyclic TAT peptides, and HA-TAT peptides, among others. In some embodiments, the polypeptides of the current invention are delivered with separate small peptides which aid and improve cell permeabilization. Examples of such cell permeabilization aids include but are not limited to Transportan, Mastoparan, KALA, Penetratin-Arg, Penetratin, or TAT-HA2 (Anaspec).

In some embodiments, the second polypeptide domain comprises a linker region. Linker regions or sequences are typically rich in glycine for flexibility, as well as serine or threonine for solubility and low steric hinderance. The linker can link the cell-penetrating domain to the DNA-speckle targeting motif domain of the polypeptides of the invention. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)n, (GSGGS)n (SEQ ID NO: 1732), (GGGS)n (SEQ ID NO: 1733), and (GGGGS)n (SEQ ID NO: 1734), where n represents an integer of at least 1. Exemplary linker sequences can comprise amino acid sequences including, without limitation, GGSG (SEQ ID NO: 1735), GGSGG (SEQ ID NO: 1736), GSGSG (SEQ ID NO: 1737), GSGGG (SEQ ID NO: 1738), GGGSG (SEQ ID NO: 1739), GSSSG (SEQ ID NO: 1740), GGGGS (SEQ ID NO: 1741), GGGGSGGGGSGGGGS (SEQ ID NO: 1742) and the like. In some preferred embodiments, the linker sequence comprises the amino acid sequence GGSGGGSG (SEQ ID NO: 1743). It is also contemplated that the length and composition of the linker region can be optimized, including expanding or contracting the GGS repeat length, and by using other linkers, such as GIHGVPAAT (SEQ ID NO: 1744). Those of skill in the art would be able to select the appropriate linker sequence for use in the present invention.

In some embodiments, the fourth polypeptide domain comprises a nuclear localization signal (NLS). The NLS will assist the peptide to access the nuclear compartment. The term “NLS” or “nuclear localization signal” as used herein refers to an amino acid sequence, which identifies a cytoplasmic protein for import into the nucleus via a nuclear transport mechanism. Typically, this signal consists of one or more short sequences of positively charged amino acids (lysine or arginine) exposed on an exterior surface of the protein. Various nuclear localized proteins may share the same NLS. Non-binding examples of NLS sequences include the amino acid sequence PKKKRKV (SEQ ID NO: 1745) in the SV40 Large T-antigen and the amino acid sequence RRARRPRG (SEQ ID NO: 1746) from VP1 of the chicken anemia virus (CAV) which are both monopartite NLS, as well as bipartite NLS sequences in which the basic amino acid residues are present in two clusters, such as in NLS of nucleoplasmin, KR[PAATKKAGQA]KKKK (SEQ ID NO: 1747). There are many other types of NLS, which are known as “non-classical”, such as the acidic M9 domain of hnRNP A1, the sequence KIPIK in yeast transcription repressor Mata2, and the complex signals of U snRNPs among others. Thus, any type of NLS known in the art (classical or non-classical) may be used in combination with the current invention in order to direct the polypeptides of the current invention in order direct import into the nucleus of a target cell.

### DNA-Speckle Targeting Motif

In some embodiments, the current invention provides a polypeptide inhibitor of transcription factor/DNA-speckle association comprising a DNA-speckle targeting motif. The speckle targeting motif (STM) is polypeptide sequence which follows a distinct and defined pattern of amino acid residues (see Experimental Example 1 and Example 2) which acts to mediate the association of the transcription factor with DNA-speckles. Speckle targeting motifs comprise the amino acid pattern, x(30)-[TS]-P-x(30), wherein x is any amino acid and that:
1. Do not contain four or more consecutive Proline residues.
2. Contain Prolines in a minimum of three of the correctly spaced positions: amino acids 16, 21, 26, 36, 41, or 46
3. At least five negative or phosphorylatable amino acids (D, E, T, S)
4. At least five small or hydrophobic amino acids (A, M, V, F, L, I)
5. Fewer than fifteen positively charged amino acids (R, H, K)

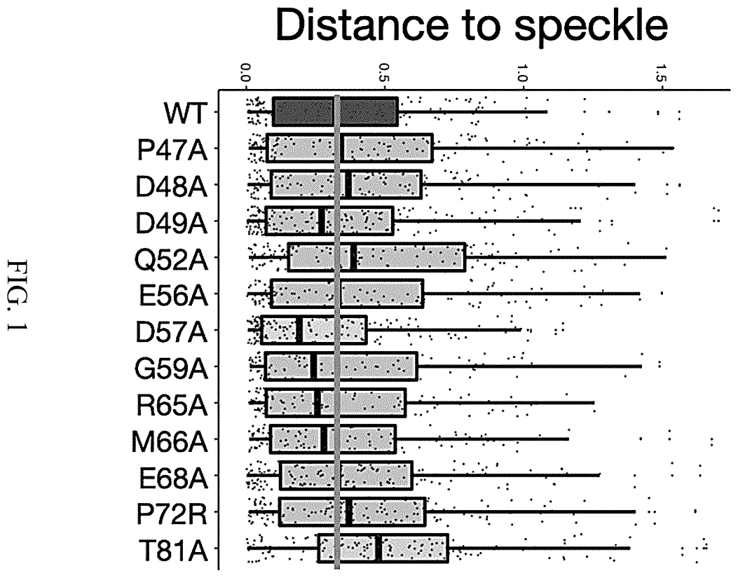

The currently defined consensus speckle targeting motif is 30 amino acids in length, spanning from amino acid 16 to amino acid 46 of the x(30)-[TS]-P-x(30) 62 amino acid peptide pattern that was extracted from the proteome (Table 1; all the speckle targeting motifs found in the genome). Here, additional amino acids to the central 30 amino acid STM are included for their potential to add specificity for individual transcription factor speckle targeting activity. Based on data that phosphorylation of the central S or T may be critical for speckle-associating functions of p53 (see Example 1; FIGS. 1 and 3), an expanded consensus speckle targeting motif is defined as x(30)-[TSED]-P-x(30), which includes the negatively charged amino acids, E and D, which would have similar biochemical properties to phosphorylated T or S (Table 2).

In some embodiments, the biochemical properties of the speckle targeting motif can be optimized to modulate speckle-targeting blocking activity including:
1. Transcription factor specificity. The specificity of the composition to each transcription factor can be optimized, starting with using the unique amino acid features of each transcription factor STM. This includes their unique x amino acid composition, proline spacing, and extending past the core STM on either or both sides. Each of these features will be optimized (see also below).
2. Proline spacing. The consensus speckle target motif constitutes the following spacing of prolines: PxxxxPxxxxPxxx[TSED]PxxxxPxxxxPxxxxP with P designating a Proline, x designating any other amino acid and [TSED] designating either a Threonine, Serine, Aspartate, or Glutamate. The number of spaced prolines, and their exact positions can be optimized.
3. Speckle targeting motif length. Starting from the full 30 amino acid speckle targeting motif, the speckle targeting motif can be shortened or lengthened on either or both sides of the TP/SP/EP/DP motif.
4. Charge and phosphomimetics. The central TSED can be optimized for charge and phosphomimickry, using T, S, E, or D as well as phospho-mimicking T and S synthetic amino acids
5. Composition of x amino acids. The complexity and biochemical properties of x amino acids can be optimized, using naturally occurring speckle targeting motifs within transcription factors as guides.
6. Proline isomerization. Proline residues are a special amino acid that covalently bond with the peptide backbone in one of two possible conformations (cis or trans). The specific conformation of each proline needed for speckle-targeting-blocking activities can be altered at each position using synthetic prolines that favor either the cis or the trans conformation.
7. Number of tandem speckle targeting motifs. The STM can be repeated from one to any number of times within the same polypeptide to accomplish maximum activity. Multiple STMs in one protein occurs naturally in several STM-containing proteins, including HIF2A and KMT2D.

TABLE 1
List of speckle targeting motif containing
proteins according to x(30)-[TS]P-x(30).
Proteins with more than one speckle targeting motif are
designated by ProteinName_[0-number of motifs minus one].

| SEQ | | |
|---|---|---|
| ID | | |
| NO: | Name: | Sequence: |
| 1 | MUC17_0 | IPVITSTEASSSPTTAEGTSIPTSTYTEGSTPLTSTPASTMPV |
| | | ATSEMSTLSITPVDTSTLV |
| 2 | MUC17_1 | STEASSSPTTAEGTSIPTSTYTEGSTPLTSTPASTMPVATSE |
| | | MSTLSITPVDTSTLVTTSTE |
| 3 | MUC17_2 | TPVTNSTEARSSPTTSEGTSMPTSTPGEGSTPLTSMPDSTTP |
| | | VVSSEARTLSATPVDTSTPV |
| 4 | MUC17_3 | TQVATSTEASSPPPTAEVTSMPTSTPGERSTPLTSMPVRHT |
| | | PVASSEASTLSTSPVDTSTPV |
| 5 | MUC17_4 | TPVTTSTEACSSPTTSEGTSMPNSNPSEGTTPLTSIPVSTTPV |
| | | VSSEASTLSATPVDTSTPG |
| 6 | MUC17_5 | TPGTTSAEATSSPTTAEGISIPTSTPSEGKTPLKSIPVSNTPV |
| | | ANSEASTLSTTPVDSNSPV |
| 7 | MUC17_6 | TPVTTSTEARSSPTTSEGTSMPNSTPSEGTTPLTSIPVSTTPV |
| | | LSSEASTLSATPIDTSTPV |
| 8 | MUC17_7 | TPVTNSTEARSSPTTSEGTSMPTSTPSEGSTPFTSMPVSTMP |
| | | VVTSEASTLSATPVDTSTPV |
| 9 | MUC17_8 | TPVTTYSQAGSSPTTADDTSMPTSTYSEGSTPLTSVPVSTM |
| | | PVVSSEASTHSTTPVDTSTPV |
| 10 | MUC17_9 | TPVTTSTEASSSPTTAEGTSIPTSPPSEGTTPLASMPVSTTPV |
| | | VSSEAGTLSTTPVDTSTPM |
| 11 | MUC17_10 | SPVVTSTEISSSATSAEGTSMPTSTYSEGSTPLRSMPVSTKP |
| | | LASSEASTLSTTPVDTSIPV |
| 12 | MUC17_11 | IPVTTSTEASSSPTTAEVTSMPTSTPSETSTPLTSMPVNHTP |
| | | VASSEAGTLSTTPVDTSTPV |
| 13 | MUC17_12 | TPVTTSTEASSSPTTAEGTGIPISTPSEGSTPLTSIPVSTTPVA |
| | | IPEASTLSTTPVDSNSPV |
| 14 | MUC17 13 | SPVVTSTEVSSSPTPAEGTSMPISTYSEGSTPLTGVPVSTTP |
| | | VTSSAISTLSTTPVDTSTPV |
| 15 | MUC17_14 | STPVTTSTEATSSTTAEGTSIPTSTPSEGMTPLTSVPVSNTP |
| | | VASSEASILSTTPVDSNTPL |
| 16 | MUC17_15 | TPVTTSTEASLSPTTAEGTSIPTSSPSEGTTPLASMPVSTTPV |
| | | VSSEVNTLSTTPVDSNTLV |
| 17 | MUC17_16 | TLVTTSTEASSSPTIAEGTSLPTSTTSEGSTPLSIMPLSTTPV |
| | | ASSEASTLSTTPVDTSTPV |
| 18 | MUC17_17 | TPVTTSSPTNSSPTTAEVTSMPTSTAGEGSTPLTNMPVSTTP |
| | | VASSEASTLSTTPVDSNTFV |
| 19 | MYO15B_0 | HRLALRLAGLAGLGGMPRASPGGRSPQVPTSPVPGDPFDQ |
| | | EDETPDPKFAVVFPRIHRAGRA |
| 20 | MYO15B_1 | AFLRKIDPKDEALAKLGINGAHSSPPMLSPSPGKGPPPAVA |
| | | PRPKAPLQLGPSSSIKEKQGP |
| 21 | FAM178B | RPCSPASAPAPTSPKKPKIQAPGETFPTDWSPPPVEFLNPRV |
| | | LQASREAPAQRWVGVVGPQG |
| 22 | INPP5J_0 | HSSPEDPVLPRPPQTLPLDVGQGPSEPGTHSPGLLSPTFRPG |
| | | APSGQTVPPPLPKPPRSPSR |
| 23 | INPP5J_1 | DPVLPRPPQTLPLDVGQGPSEPGTHSPGLLSPTFRPGAPSG |
| | | QTVPPPLPKPPRSPSRSPSHS |
| 24 | COL15A1 | VAEILEAVTYTQASPKEAKVEPINTPPTPSSPFEDMELSGEP |
| | | VPEGTLETTNMSIIQHSSPK |
| 25 | SH3RF1 | PTAAARISELSGLSCSAPSQVHISTTGLIVTPPPSSPVTTGPS |
| | | FTFPSDVPYQAALGTLNPP |
| 26 | EZHIP | DENPSCGTGSERLAFQSRSGSPDPEVPSRASPPVWHAVRM |
| | | RASSPSPPGRFFLPIPQQWDES |
| 27 | CTAGE1 | EFKIKLLEKDPYGLDVPNTAFGRQHSPYGPSPLGWPSSETR |
| | | ASLYPPTLLEGPLRLSPLLPR |
| 28 | BPTF_0 | PTHAQSSKPQVAAQSQPQSNVQGQSPVRVQSPSQTRIRPST |
| | | PSQLSPGQQSQVQTTTSQPIP |
| 29 | BPTF_1 | QPQSNVQGQSPVRVQSPSQTRIRPSTPSQLSPGQQSQVQTT |
| | | TSQPIPIQPHTSLQIPSQGQP |
| 30 | NRXN3 | KMNNRDLKPQPDIVLLPLPTAYELDSTKLKSPLITSPMFRN |
| | | VPTANPTEPGIRRVPGASEVI |
| 31 | ANKHD1-EIF4EBP3 | PHFALLAAQTMQQIRHPRLPMAQFGGTFSPSPNTWGPFPV |
| | | RPVNPGNTNSSPKHNNTSRLPN |
| 32 | putative | LCLIPRNTGTPQRVLRPVVWSPPSRKKPVLSPHNSIMFGHL |
| | | SPVRIPCLRGKFNLQLPSLDD |
| 33 | C1orf94 | KNVLDKTRVTKDFLQDNLFSGPGPKEPTGLSPFLLLPPRPP |
| | | PARPDKLPELPAQKRQLPVFA |
| 34 | ITIH6_0 | KPGSLSHQNPDILPTNSRTQVPPVKPGIPASPKADTVKCVT |
| | | PLHSKPGAPSHPQLGALTSQA |
| 35 | ITIH6_1 | LSKTPKILLSLKPSAPPHQISTSISLSKPETPNPHMPQTPLPP |
| | | RPDRPRPPLPESLSTFPNT |
| 36 | KIAA1614 | GSINEEQPARDGGPRLPRPPAPGREYCNRGSPWPPEAEWT |
| | | LPDHDRGPLLGPSSLQQSPIHG |
| 37 | KRTAP10-10 | CTDSWRVVDCPESCCEPCCCAPAPSLTLVCTPVSCVSSPCC |
| | | QTACEPSACQSGYTSSCTTPC |
| 38 | IFITM10 | LGDPASTTDGAQEARVPLDGAFWIPRPPAGSPKGCFACVS |
| | | KPPALQAPAAPAPEPSASPPMA |
| 39 | MS4A15 | GLCPPPAILPTSMCQPPGIMQFEEPPLGAQTPRATQPPDLRP |
| | | VETFLTGEPKVLGTVQILIG |
| 40 | SP5 | PQKTHLQPSFGAAHELPLTPPADPSYPYEFSPVKMLPSSMA |
| | | ALPASCAPAYVPYAAQAALPP |
| 41 | FOXE1 | AARPPYPGAVYAGYAPPSLAAPPPVYYPAASPGPCRVFGL |
| | | VPERPLSPELGPAPSGPGGSCA |
| 42 | PRICKLE2 | EYAWVPPGLKPEQVHQYYSCLPEEKVPYVNSPGEKLRIKQ |
| | | LLHQLPPHDNEVRYCNSLDEEE |
| 43 | C7orf26_0 | LCTRDDLRTLCSRLPHNNLLQLVISGPVQQSPHAALPPGFY |
| | | PHIHTPPLGYGAVPAHPAAHP |
| 44 | C7orf26_1 | HNNLLQLVISGPVQQSPHAALPPGFYPHIHTPPLGYGAVPA |
| | | HPAAHPALPTHPGHTFISGVT |
| 45 | MAGEB17_0 | EKRRQARGEDQCLGGAQATAAEKEKLPSSSSPACQSPPQS |
| | | FPNAGIPQESQRASYPSSPASA |
| 46 | MAGEB17_1 | ARGEDQCLGGAQATAAEKEKLPSSSSPACQSPPQSFPNAGI |
| | | PQESQRASYPSSPASAVSLTS |
| 47 | ATP6V1FNB | ARLPLKLPTLHPKAPLSPPPAPKSAPSKVPSPVPEAPFQSEM |
| | | YPVPPITRALLYEGISHDFQ |
| 48 | PCDH9 | ATDGGQPPRSSTAKVTINVMDVNDNSPVVISPPSNTSFKLV |
| | | PLSAIPGSVVAEVFAVDVDTG |
| 49 | FAM131C | YLQDSLPSGPSQDDSLQAFSSPSPSPDSCPSPEEPPSTAGIPQ |
| | | PPSPELQHRRRLPGAQGPE |
| 50 | FAM221B | SAEDLQENHISESFLKPSTSETPLEPHTSESPLVPSPSQIPLE |
| | | AHSPETHQEPSISETPSET |
| 51 | TOX3 | QQLQQQLQQRLQLQQLQHMQHQSQPSPRQHSPVASQITSP |
| | | IPAIGSPQPASQQHQSQIQSQT |
| 52 | MAMSTR | EQISDPDPWISASDPPLAPALPSGTAPFLFSPGVLLPEPEYC |
| | | PPWRSPKKESPKISQRWRES |
| 53 | ZAN | CAQAGQAPAWRNRTFCPMRCPPGSSYSPCSSPCPDTCSSIN |
| | | NPRDCPKALPCAESCECQKGH |
| 54 | PCLO_0 | RPQTKQADIVRGESVKPSLPSPSKPPIQQPTPGKPPAQQPG |
| | | HEKSQPGPAKPPAQPSGLTKP |
| 55 | PCLO_1 | KTPAQQPGPAKPPTQQVGTPKPLAQQPGLQSPAKAPGPTK |
| | | TPVQQPGPGKIPAQQAGPGKTS |
| 56 | PCLO_2 | KPPTQQVGTPKPLAQQPGLQSPAKAPGPTKTPVQQPGPGK |
| | | IPAQQAGPGKTSAQQTGPTKPP |
| 57 | C22orf23 | IMDIMKRGDALPLQCSPTSSQRVLPSKQIASPIYLPPILAAR |
| | | PHLRPANMCQANGAYSREQF |
| 58 | HSFX1 | RNSRGQDHGLERVPFPPQLQSETYLHPADPSPAWDDPGST |
| | | GSPNLRLLTEEIAFQPLAEEAS |
| 59 | FAM13C | RNLLCEQPTVPRENGKPEAAGPEPSSSGEETPDAALTCLKE |
| | | RREQLPPQEDSKVTKQDKNLI |
| 60 | THAP8_0 | PLQKNTPLPQSPAIPVSGPVRLVVLGPTSGSPKTVATMLLT |
| | | PLAPAPTPERSQPEVPAQQAQ |
| 61 | THAP8_1 | SPAIPVSGPVRLVVLGPTSGSPKTVATMLLTPLAPAPTPER |
| | | SQPEVPAQQAQTGLGPVLGAL |
| 62 | PRR27 | VPPSRFFSAAAAPAAPPIAAEPAAAAPLTATPVAAEPAAG |
| | | APVAAEPAAEAPVGAEPAAEAP |
| 63 | LRRN4 | VLEPDISAASTPLASKLLGPFPTSWDRSISSPQPGQRTHATP |
| | | QAPNPSLSEGEIPVLLLDDY |
| 64 | KDF1 | QRLKSTMGSSFSYPDVKLKGIPVYPYPRATSPAPDADSCC |
| | | KEPLADPPPMRHSLPSTFASSP |
| 65 | NEXMIF | INGVKENDSEDQDVAMKSFAALEAAAPIQPTPVAQKETL |
| | | MYPRGLLPLPSKKPCMQSPPSPL |
| 66 | KLHDC7B | PGGGWPWVSREVPGTRSFGPAPDSTRPWLESPPQGRPLSS |
| | | QGPGATGAYDAGEAGADSSRDN |
| 67 | C19orf67 | EGSLPLDPGETPPPDALEPGTPPCGDPSRSTPPGRPGNPSEP |
| | | DPEDAEGRLAEARASTSSPK |
| 68 | RAB44_0 | TAHSELPQQDSLLVSLPSATPQAQVEAEGPTPGKSAPPRGS |
| | | PPRGAQPGAGAGPQEPTQTPP |
| 69 | RAB44_1 | SLLVSLPSATPQAQVEAEGPTPGKSAPPRGSPPRGAQPGAG |
| | | AGPQEPTQTPPTMAEQEAQPR |
| 70 | ZNF341_0 | SGTVEIQALGMQPYPPLEVPNQCVEPPVYPTPTVYSPGKQ |
| | | GFKPKGPNPAAPMTSATGGTVA |
| 71 | ZNF341_1 | IQALGMQPYPPLEVPNQCVEPPVYPTPTVYSPGKQGFKPK |
| | | GPNPAAPMTSATGGTVATFDSP |
| 72 | RTL10 | KEPPVLPSSTCSSKPGPVEPASSQPEEAAPTPVPRLSESANP |
| | | PAQRPDPAHPGGPKPQKTEE |
| 73 | IQCN_0 | KTLLQTYPVVSVTLPQTYPASTMTTTPPKTSPVPKVTIIKTP |
| | | AQMYPGPTVTKTAPHTCPMP |
| 74 | IQCN_1 | SVTLPQTYPASTMTTTPPKTSPVPKVTIIKTPAQMYPGPTV |
| | | TKTAPHTCPMPTMTKIQVHPT |
| 75 | ZNF653 | SPVGSSGLITQEGVHIPFDVHHVESLAEQGTPLCSNPAGNG |
| | | PEALETVVCVPVPVQVGAGPS |
| 76 | KRTAP10-11 | QVDDCPESCCEPPCSAPSCCAPAPSLSLVCTPVSCVSSPCC |
| | | QAACEPSACQSGCTSSCTPSC |
| 77 | TTBK1 | TNSLPNGPALADGPAPVSPLEPSPEKVATISPRRHAMPGSR |
| | | PRSRIPVLLSEEDTGSEPSGS |
| 78 | CCDC184 | GRDPEDEEEEEEEKEMPSPATPSSHCERPESPCAGLLGGDG |
| | | PLVEPLDMPDITLLQLEGEAS |
| 79 | UBQLN3_0 | QSLGTYLQGTASALSQSQEPPPSVNRVPPSSPSSQEPGSGQ |
| | | PLPEESVAIKGRSSCPAFLRY |
| 80 | UBQLN3_1 | SSTGHSTNLPDLVSGLGDSANRVPFAPLSFSPTAAIPGIPEP |
| | | PWLPSPAYPRSLRPDGMNPA |
| 81 | PRDM8 | STPAAASPVGAEKLLAPRPGGPLPSRLEGGSPARGSAFTSV |
| | | PQLGSAGSTSGGGGTGAGAAG |
| 82 | PCBP4 | GTPSSAPADLPAPFSPPLTALPTAPPGLLGTPYAISLSNFIGL |
| | | KPMPFLALPPASPGPPPGL |
| 83 | RNF222_0 | KSSQTLAVPVGLPSVPPLDSLGHTNPLAASSPAWRPPPGQ |
| | | ARPPGSPGQSAQLPLDLLPSLP |
| 84 | RNF222_1 | PPLDSLGHTNPLAASSPAWRPPPGQARPPGSPGQSAQLPLD |
| | | LLPSLPRESQIFVISRHGMPL |
| 85 | ARMCX5 | ARYIVLVPVEGGEQSLPPEGNWTLVETLIETPLGIRPLTKIP |
| | | PYHGPYYQTLAEIKKQIRQR |
| 86 | DNM1 | RPGSRGPAPGPPPAGSALGGAPPVPSRPGASPDPFGPPPQV |
| | | PSRPNRAPPGVPSRSGQASPS |
| 87 | ZNF541 | EACGDSPHAHESAGQPPPSSLRSLVPPEARSPGSLLPHRDL |
| | | LRRIVSSIVHQKTPSPGPAPA |
| 88 | FMN1_0 | PAPAALGKVFNNSASQSSTHKQTSPVPSPLSPRLPSPQQHH |
| | | RILRLPALPGEREAALNDSPC |
| 89 | FMN1_1 | LGKVFNNSASQSSTHKQTSPVPSPLSPRLPSPQQHHRILRLP |
| | | ALPGEREAALNDSPCRKSRV |
| 90 | FBXO41 | LFARKSVASSACSTPPPGPGPGPCPGPASASPASPSPADVA |
| | | YEEGLARLKIRALEKLEVDRR |
| 91 | GAS2L2 | TKASLSAKGTHMRKVPPQGGQDCSASTVSASPEAPTPSPL |
| | | DPNSDKAKACLSKGRRTLRKPK |
| 92 | UBAP1L | VSRPRALLHGLRGHRALSLCPSPAQSPRSASPPGPAPQHPA |
| | | APASPPRPSTAGAIPPLRSHK |
| 93 | IGSF9B_0 | PFHHGQYYGYLSSSSPGEVEPPPFYVPEVGSPLSSVMSSPP |
| | | LPTEGPFGHPTIPEENGENAS |
| 94 | IGSF9B_1 | NSTLPLTQTPTGGRSPEPWGRPEFPFGGLETPAMMFPHQLP |
| | | PCDVPESLQPKAGLPRGLPPT |
| 95 | ATF7-NPFF | GCGMVVGTASTMVTARPEQSQILIQHPDAPSPAQPQVSPA |
| | | QPTPSTGGRRRRTVDEDPDERR |
| 96 | HSFX2 | RNSRGQDHGLERVPFPPQLQSETYLHPADPSPAWDDPGST |
| | | GSPNLRLLTEEIAFQPLAEEAS |
| 97 | NPIPB6 | PPSVDDNLKECLFVPLPPSPLPPSVDDNLKTPPLATQEAEV |
| | | EKPPKPKRWRVDEVEQSPKPK |
| 98 | PCED1B | HSDVPSSAHAGFFVEDNFMVGPQLPMPFFPTPRYQRPAPV |
| | | VHRGFGRYRPRGPYTPWGQRPR |
| 99 | NPIPB9 | PPSVDDNLKECLFVPLPPSPLPPSVDDNLKTPPLATQEAEV |
| | | EKPPKPKRWRVDEVEQSPKPK |
| 100 | SLFNL1 | DLLLSEAQGPFSHREEKEEEEEDSGLSPGPSPGSGVPLPTW |
| | | PTHTLPDRPQAQQLQSCQGRP |
| 101 | NLGN4Y | HNLNEIFQYVSTTTKVPPPDMTSFPYGTRRSPAKIWPTTKR |
| | | PAITPANNPKHSKDPHKTGPE |
| 102 | PRRT4 | VALPLALLGLYPALCSPRVPPRCWAKLFRLSPGHAAPLLP |
| | | GGWVTGPPDKEPLGSAIARGDA |
| 103 | NUTM1 | PALPFLPPTSDPPDHPPREPPPQPIMPSVFSPDNPLMLSAFPS |
| | | SLLVTGDGGPCLSGAGAGK |
| 104 | LMTK3_0 | VSENGGLRFPRNTERPPETGPWRAPGPWEKTPESWGPAPT |
| | | IGEPAPETSLERAPAPSAVVSS |
| 105 | LMTK3_1 | PTNELSVQAPPEGDTDPSTPPAPPTPPHPATPGDGFPSNDS |
| | | GFGGSFEWAEDFPLLPPPGPP |
| 106 | ZCCHC14 | SSLNGGGGHGGKGAPGPGGALPTCPACHKITPRTEAPVSS |
| | | VSNSLENALHTSAHSTEESLPK |
| 107 | MIA2 | ELKFELLEKDPYALDVPNTAFGREHSPYGPSPLGWPSSETR |
| | | AFLSPPTLLEGPLRLSPLLPG |
| 108 | CTNND2 | AAAAAALYYSSSTLPAPPRGGSPLAAPQGGSPTKLQRGGS |
| | | APEGATYAAPRGSSPKQSPSRL |
| 109 | NRG3 | SRTPNRISTRLTTITRAPTRFPGHRVPIRASPRSTTARNTAA |
| | | PATVPSTTAPFFSSSTLGSR |
| 110 | KCNC2 | KTLPGTRLALLASSEPPGDCLTTAGDKLQPSPPPLSPPPRAP |
| | | PLSPGPGGCFEGGAGNCSSR |
| 111 | CD300E_0 | DAGSYWCKIQTVWVLDSWSRDPSDLVRVYVSPAITTPRR |
| | | TTHPATPPIFLVVNPGRNLSTGE |
| 112 | CD300E_1 | WCKIQTVWVLDSWSRDPSDLVRVYVSPAITTPRRTTHPAT |
| | | PPIFLVVNPGRNLSTGEVLTQN |
| 113 | COL9A1 | SVPFELQWMLIHCDPLRPRRETCHELPARITPSQTTDERGP |
| | | PGEQGPPGPPGPPGVPGIDGI |
| 114 | HTR3E | TIFITHLLHVATTQPPPLPRWLHSLLLHCNSPGRCCPTAPQ |
| | | KENKGPGLTPTHLPGVKEPEV |
| 115 | NPIPB15 | PPSVDDNLKDCLFVPLPPSPLPPSVDDNLKTPPLATQEAEA |
| | | EKPPKPKRWRVDEVEQSPKPK |
| 116 | SPEM3 | HLVRSSVPVPTSAPAPPGTLAPATTPVLAPTPAPVPASAPSP |
| | | APALVMALTTTPVPDPVPAT |
| 117 | KRTAP10-4 | QVDDCPESCCEPPCCAPSCCAPAPCLSLVCTPVSRVSSPCC |
| | | PVTCEPSPCQSGCTSSCTPSC |
| 118 | CRIP3 | GVNIGGVGSYLYNPPTPSPGCTTPLSPSSFSPPRPRTGLPQG |
| | | KKSPPHMKTFTGETSLCPGC |
| 119 | LRRC37A2 | PEHSHLTQATVQPLDLGFTITPESKTEVELSPTMKETPTQPP |
| | | KKVVPQLRVYQGVTNPTPGQ |
| 120 | KRTAP10-6 | CSDSWQVDDCPESCCEPPCCAPAPCLSLVCTPVSRVSSPCC |
| | | PVTCEPSPCQSGCTSSCTPSC |
| 121 | PNMA5 | GRSMTDVARALGCCSLPAESLDAEVMPQVRSPPLEPPKES |
| | | MWYRKLKVFSGTASPSPGEETF |
| 122 | ZNF683 | LLPYPGAFQASGQALPSQARNPGAGAAPTDSPGLERGGM |
| | | ASPAKRVPLSSQTGTAALPYPLK |
| 123 | PRR23A | CALAPNPSSEGHSPGPFFDPEFRLLEPVPSSPLQPLPPSPRV |
| | | GSPGPHAHPPLPKRPPCKAR |
| 124 | SELENOV_0 | PTPLRTPTPVRTRTPIRTLTPVLTPSPAGTSPLVLTPAPAQIP |
| | | TLVPTPALARIPRLVPPPA |
| 125 | SELENOV_1 | TPTPVRTRTPIRTLTPVLTPSPAGTSPLVLTPAPAQIPTLVPT |
| | | PALARIPRLVPPPAPAWIP |
| 126 | SELENOV_2 | ALARIPRLVPPPAPAWIPTPVPTPVPVRNPTPVPTPARTLTP |
| | | PVRVPAPAPAQLLAGIRAAL |
| 127 | STON1-GTF2A1L_0 | EFPSGSSSTSSTPLSSPIVDFYFSPGPPSNSPLSTPTKDFPGFP |
| | | GIPKAGTHVLYPIPESSS |
| 128 | STON1-GTF2A1L_1 | ISGGESSLLPTRPTCLSHALLPSDHSCTHPTPKVGLPDEVNP |
| | | QQAESLGFQSDDLPQFQYFR |
| 129 | POC1B-GALNT4 | AVVVVTGRRCRSGQTVPGAARSPLLPHPLPSPLRVPPPTG |
| | | ALGRPLPRWPQPRRTPFWSVIS |
| 130 | IKZF5 | PTSPEPRPSHSQRNYSPVAGPSSEPSAHTSTPSIGNSQPSTPA |
| | | PALPVQDPQLLHHCQHCDM |
| 131 | RHBDD3 | SCGYMPVHLAMLAGEGHRPRRPRGALPPWLSPWLLLALT |
| | | PLLSSEPPFLQLLCGLLAGLAYA |
| 132 | PRR23C | CALAPNPSSERRSPRPIFDLEFHLLEPVPSSPLQPLPPSPSPG |
| | | PHARPELPERPPCKVRRRL |
| 133 | PRR23B | CALAPNPSSERRSPRPIFDLEFRLLEPVPSSPLQPLPPSPCVG |
| | | SPGPHARSPLPERPPCKAR |
| 134 | STRC | GSNRRLVKRLCAGLLPPPTSCPEGLPPVPLTPDIFWGCFLE |
| | | NETLWAERLCGEASLQAVPPS |
| 135 | NKX1-1_0 | NPGADTSAPTGGGGGPGPGAGPGTGLPGGLSPLSPSPPMG |
| | | APLGMHGPAGYPAHGPGGLVCA |
| 136 | NKX1-1_1 | TSAPTGGGGGPGPGAGPGTGLPGGLSPLSPSPPMGAPLGM |
| | | HGPAGYPAHGPGGLVCAAQLPF |
| 137 | HCFC1R1 | ATHFSQLSLHNDHPYCSPPMTFSPALPPLRSPCSELLLWRY |
| | | PGSLIPEALRLLRLGDTPSPP |
| 138 | SPATA31A3 | SLSASQPPEPSLPLEHPSPEPPALFPHPPHTPDPLACSLPPPK |
| | | GFTAPPLRDSTLITPSHCD |
| 139 | OTUD4 | TCTDAHFPMQTEASVNGQMPQPEIGPPTFSSPLVIPPSQVS |
| | | ESHGQLSYQADLESETPGQLL |
| 140 | LRRC37A | PEHSHLTQATVQPLDLGFTITPESKTEVELSPTMKETPTQPP |
| | | KKVVPQLRVYQGVTNPTPGQ |
| 141 | FOXB2 | PEYGAFGVPVKSLCHSASQSLPAMPVPIKPTPALPPVSALQ |
| | | PGLTVPAASQQPPAPSTVCSA |
| 142 | KRTAP10-8 | SPSTCTGSSWQVDNCQESCCEPRSCASSCCTPSCCAPAPCL |
| | | ALVCAPVSCEPSPCQSGCTDS |
| 143 | KRTAP10-12 | CSDSWQVDDCPESCCEPPCCAPAPCLSLVCTPVSRVSSPCC |
| | | RVTCEPSPCQSGCTSSCTPSC |
| 144 | PLAGL2 | PPGATGGLVMGYSQAEAQPLLTTLQAQPQDSPGAGGPLN |
| | | FGPLHSLPPVFTSGLSSTTLPRF |
| 145 | CCDC187_0 | AGQACSPQRAWGAQRQGPSSQRPGSPPEKRSPFPQQPWS |
| | | AVATQPCPRRAWTACETWEDPGP |
| 146 | CCDC187_1 | DTVRDPAVGLLRSCPHSLPAAPTLATPTLATPACPGALGP |
| | | NWGRGAPGEWVSMQPQPLLPPT |
| 147 | SPATA31A7 | SLSASQPPEPSLPLEHPSPEPPALFPHPPHTPDPLACSLPPPK |
| | | GFTAPPLRDSTLITPSHCD |
| 148 | NOBOX | LEELEPQDYQQSNQPGPFQFSQAPQPPLFQSPQPKLPYLPT |
| | | FPFSMPSSLTLPPPEDSLFMF |
| 149 | TTN_0 | LSATSSAQKITKSVKAPTVKPSETRVRAEPTPLPQFPFADTP |
| | | DTYKSEAGVEVKKEVGVSIT |
| 150 | TTN_1 | PAAPLGAPTYIPTLEPVSRIRSLSPRSVSRSPIRMSPARMSPA |
| | | RMSPARMSPARMSPGRRLE |
| 151 | TTN_2 | GAPTYIPTLEPVSRIRSLSPRSVSRSPIRMSPARMSPARMSP |
| | | ARMSPARMSPGRRLEETDES |
| 152 | TTN_3 | IPTLEPVSRIRSLSPRSVSRSPIRMSPARMSPARMSPARMSP |
| | | ARMSPGRRLEETDESQLERL |
| 153 | TTN_4 | PVSRIRSLSPRSVSRSPIRMSPARMSPARMSPARMSPARMS |
| | | PGRRLEETDESQLERLYKPVF |
| 154 | TTN_5 | RSLSPRSVSRSPIRMSPARMSPARMSPARMSPARMSPGRR |
| | | LEETDESQLERLYKPVFVLKPV |
| 155 | TTN_6 | RSVSRSPIRMSPARMSPARMSPARMSPARMSPGRRLEETD |
| | | ESQLERLYKPVFVLKPVSFKCL |
| 156 | TTN_7 | PEVPPTKVPEVPKAAVPEKKVPEAIPPKPESPPPEVPEAPKE |
| | | VVPEKKVPAAPPKKPEVTPV |
| 157 | TTN_8 | PEVPPTKVPEVPKVAVPEKKVPEAIPPKPESPPPEVFEEPEE |
| | | VALEEPPAEVVEEPEPAAPP |
| 158 | TTN_9 | IELMRPVSELIRSRPQPAEEYEDDTERRSPTPERTRPRSPSP |
| | | VSSERSLSRFERSARFDIFS |
| 159 | TTN_10 | EKAVTSPPRVKSPEPRVKSPEAVKSPKRVKSPEPSHPKAVS |
| | | PTETKPTPTEKVQHLPVSAPP |
| 160 | TTN_11 | KSPEPRVKSPEAVKSPKRVKSPEPSHPKAVSPTETKPTPTE |
| | | KVQHLPVSAPPKITQFLKAEA |
| 161 | KIF26B | ESDKEDNGSEGQLTNREGPELPASKMQRSHSPVPAAAPAH |
| | | SPSPASPRSVPGSSSQHSASPL |
| 162 | COL16A1 | NSGEKGDQGFQGQPGFPGPPGPPGFPGKVGSPGPPGPQAE |
| | | KGSEGIRGPSGLPGSPGPPGPP |
| 163 | ESAM_0 | DTISKNGTLSSVTSARALRPPHGPPRPGALTPTPSLSSQALP |
| | | SPRLPTTDGAHPQPISPIPG |
| 164 | ESAM_1 | TSARALRPPHGPPRPGALTPTPSLSSQALPSPRLPTTDGAHP |
| | | QPISPIPGGVSSSGLSRMGA |
| 165 | DUSP8_0 | QLLEYERSLKLLAALQGDPGTPSGTPEPPPSPAAGAPLPRL |
| | | PPPTSESAATGNAAAREGGLS |
| 166 | DUSP8_1 | DIKSAYAPSRRPDGPGPPDPGEAPKLCKLDSPSGAALGLSS |
| | | PSPDSPDAAPEARPRPRRRPR |
| 167 | DUSP8_2 | RPDGPGPPDPGEAPKLCKLDSPSGAALGLSSPSPDSPDAAP |
| | | EARPRPRRRPRPPAGSPARSP |
| 168 | DUSP8_3 | GPPDPGEAPKLCKLDSPSGAALGLSSPSPDSPDAAPEARPR |
| | | PRRRPRPPAGSPARSPAHSLG |
| 169 | DUSP8_4 | PRHGLSALSAPGLPGPGQPAGPGAWAPPLDSPGTPSPDGP |
| | | WCFSPEGAQGAGGVLFAPFGRA |
| 170 | DUSP8_5 | SALSAPGLPGPGQPAGPGAWAPPLDSPGTPSPDGPWCFSP |
| | | EGAQGAGGVLFAPFGRAGAPGP |
| 171 | SULT1A2 | KCHRAPIFMRVPFLEFKVPGIPSGMETLKNTPAPRLLKTHL |
| | | PLALLPQTLLDQKVKVVYVAR |
| 172 | GPR150 | TVLGVACGHLLSVWWRHRPQAPAAAAPWSASPGRAPAP |
| | | SALPRAKVQSLKMSLLLALLFVGC |
| 173 | DRAP1 | SEDTDTDGEEETSQPPPQASHPSAHFQSPPTPFLPFASTLPL |
| | | PPAPPGPSAPDEEDEEDYDS |
| 174 | IQCE | FRGHLTRTKLLASKAHGSEPPSVPGLPDQSSPVPRVPSPIA |
| | | QATGSPVQEEAIVIIQSALRA |
| 175 | SOX13 | INLLQQQIQQVNMPYVMIPAFPPSHQPLPVTPDSQLALPIQ |
| | | PIPCKPVEYPLQLLHSPPAPV |
| 176 | CEP170B_0 | QDFMAQCLRESSPAARPSPEKVPPVLPAPLTPHGTSPVGPP |
| | | TPPPAPTDPQLTKARKQEEDD |
| 177 | CEP170B_1 | QCLRESSPAARPSPEKVPPVLPAPLTPHGTSPVGPPTPPPAP |
| | | TDPQLTKARKQEEDDSLSDA |
| 178 | MAGEC2 | STSSSLILGGPEEEEVPSGVIPNLTESIPSSPPQGPPQGPSQSP |
| | | LSSCCSSFSWSSFSEESS |
| 179 | COL22A1 | GLPGLKGDRGEKGEAGPAGPPGLPGTTSLFTPHPRMPGEQ |
| | | GPKGEKGDPGLPGEPGLQGRPG |
| 180 | EFCAB6 | EKEGMSYLDFAAGFEDPPMRGPETTPPQPPTPSKSYVNSH |
| | | FITAEECLKLFPRRLKESFRDP |
| 181 | BEND4 | PNPSSASEYGHLADVDPLSTSPVHTLGGWTSPATSESHGH |
| | | PSSSTLPEEEEEEDEEGYCPRC |
| 182 | ATRIP | LKVLVKLAENTSCDFLPRFQCVFQVLPKCLSPETPLPSVLL |
| | | AVELLSLLADHDQLAPQLCSH |
| 183 | NCAN | NRVEAHGEATATAPPSPAAETKVYSLPLSLTPTGQGGEAM |
| | | PTTPESPRADFRETGETSPAQV |
| 184 | SYNE4 | TLGQDSLGPPEHFQGGPRGNEPAAHPPRWSTPSSYEDPAG |
| | | GKHCEHPISGLEVLEAEQNSLH |
| 185 | ATAT1_0 | DIKPYSSSDREFLKVAVEPPWPLNRAPRRATPPAHPPPRSS |
| | | SLGNSPERGPLRPFVPEQELL |
| 186 | ATAT1_1 | AVEPPWPLNRAPRRATPPAHPPPRSSSLGNSPERGPLRPFV |
| | | PEQELLRSLRLCPPHPTARLL |
| 187 | TESK1 | KIKLLDTPSKPVLPLVPPSPFPSTQLPLVTTPETLVQPGTPA |
| | | RRCRSLPSSPELPRRMETAL |
| 188 | MYBPHL | AAGSKLKVKEASPADAEPPQASPGQGAGSPTPQLLPPIEEH |
| | | PKIWLPRALRQTYIRKVGDTV |
| 189 | DENND2C | SEDNIYEDIIYPTKENPYEDIPVQPLPMWRSPSAWKLPPAK |
| | | SAFKAPKLPPKPQFLHRKTME |
| 190 | PTPN4_0 | DHMVHTSPSEVFVNQRSPSSTQANSIVLESSPSQETPGDGK |
| | | PPALPPKQSKKNSWNQIHYSH |
| 191 | PTPN4_1 | TSPSEVFVNQRSPSSTQANSIVLESSPSQETPGDGKPPALPP |
| | | KQSKKNSWNQIHYSHSQQDL |
| 192 | MYCL | HYFYDYDCGEDFYRSTAPSEDIWKKFELVPSPPTSPPWGL |
| | | GPGAGDPAPGIGPPEPWPGGCT |
| 193 | FAM110A_0 | PCRRPQLDLDILSSLIDLCDSPVSPAEASRTPGRAEGAGRPP |
| | | PATPPRPPPSTSAVRRVDVR |
| 194 | FAM110A_1 | GAGRPPPATPPRPPPSTSAVRRVDVRPLPASPARPCPSPGP |
| | | AAASSPARPPGLQRSKSDLSE |
| 195 | SSC5D_0 | VCAGQRVANSRDDSTSPLDGAPWPGLLLELSPSTEEPLVT |
| | | HAPRPAGNPQNASRKKSPRPKQ |
| 196 | SSC5D_1 | TAGKLGPTLGAGTTRSPGSPPTLRVHGDTGSPRKPWPERR |
| | | PPRPAATRTAPPTPSPGPSASP |
| 197 | SSC5D_2 | NPDLILTSPDFALSTPDSSVVPALTPEPSPTPLPTLPKELTSD |
| | | PSTPSEVTSLSPTSEQVPE |
| 198 | SSC5D_3 | PALESSPSRSSTATSMDPLSTEDFKPPRSQSPNLTPPPTHTP |
| | | HSASDLTVSPDPLLSPTAHP |
| 199 | SSC5D_4 | STATSMDPLSTEDFKPPRSQSPNLTPPPTHTPHSASDLTVSP |
| | | DPLLSPTAHPLDHPPLDPLT |
| 200 | SSC5D_5 | TEDFKPPRSQSPNLTPPPTHTPHSASDLTVSPDPLLSPTAHP |
| | | LDHPPLDPLTLGPTPGQSPG |
| 201 | SSC5D_6 | SDLTVSPDPLLSPTAHPLDHPPLDPLTLGPTPGQSPGPHGPC |
| | | VAPTPPVRVMACEPPALVEL |
| 202 | PTPRN_0 | GGVVNVGADIKKTMEGPVEGRDTAELPARTSPMPGHPTA |
| | | SPTSSEVQQVPSPVSSEPPKAAR |
| 203 | PTPRN_1 | RDTAELPARTSPMPGHPTASPTSSEVQQVPSPVSSEPPKAA |
| | | RPPVTPVLLEKKSPLGQSQPT |
| 204 | SOX30_0 | PTTVYPYRSPTYSVVIPSLQNPITHPVGETSPAIQLPTPAVQ |
| | | SPSPVTLFQPSVSSAAQVAV |
| 205 | SOX30_1 | HARFATSTIQPPREYSSVSPCPRSAPIPQASPIPHPHVYQPPP |
| | | LGHPATLFGTPPRFSFHHP |
| 206 | CSPG4 | AGRVTYGATARASEAVEDTFRFRVTAPPYFSPLYTFPIHIG |
| | | GDPDAPVLTNVLLVVPEGGEG |
| 207 | RP1L1 | SPQVSLGDGQSEEASESSSPVPEDRPTPPPSPGGDTPHQRP |
| | | GSQTGPSSSRASSWGNCWQKD |
| 208 | C3orf22 | DSNTVQLPLQKRLVPTRSIPVRGLGAPDFTSPSGSCPAPLP |
| | | APSPPPLCNLWELKLLSRRFP |
| 209 | COL19A1_0 | GIGIPGRTGAQGPAGEPGIQGPRGLPGLPGTPGTPGNDGVP |
| | | GRDGKPGLPGPPGDPIALPLL |
| 210 | COL19A1_1 | SQGERGKPGLTGMKGAIGPMGPPGNKGSMGSPGHQGPPG |
| | | SPGIPGIPADAVSFEEIKKYINQ |
| 211 | KCNH5 | QLLSCRMTALEKQVAEILKILSEKSVPQASSPKSQMPLQVP |
| | | PQIPCQDIFSVSRPESPESDK |
| 212 | FAM110D | QVIARRQEPALRGSPGPLTPHPCNELGPPASPRTPRPVRRG |
| | | SGRRLPRPDSLIFYRQKRDCK |
| 213 | RUSC1 | HELAQKRKRGPGLPLVPQAKKDRSDWLIVFSPDTELPPSG |
| | | SPGGSSAPPREVTTFKELRSRS |
| 214 | PCARE_0 | RKASPTRTHWVPQADKRRRSLPSSYRPAQPSPSAVQTPPSP |
| | | PVSPRVLSPPTTKRRTSPPHQ |
| 215 | PCARE_1 | ADKRRRSLPSSYRPAQPSPSAVQTPPSPPVSPRVLSPPTTKR |
| | | RTSPPHQPKLPNPPPESAPA |
| 216 | PCARE_2 | KVSGNTHSIFCPATSSLFEAKPPLSTAHPLTPPSLPPEAGGP |
| | | LGNPAECWKNSSGPWLRADS |
| 217 | RASSF7 | AALGCEPRKTLTPEPAPSLSRPGPAAPVTPTPGCCTDLRGL |
| | | ELRVQRNAEELGHEAFWEQEL |
| 218 | MAN2B1 | ALGFSTYSVAQVPRWKPQARAPQPIPRRSWSPALTIENEHI |
| | | RATFDPDTGLLMEIMNMNQQL |
| 219 | EPX | RRPLLGASNQALARWLPAEYEDGLSLPFGWTPSRRRNGFL |
| | | LPLVRAVSNQIVRFPNERLTSD |
| 220 | NCCRP1_0 | EVREGHALGGGMEADGPASLQELPPSPRSPSPPPSPPPLPSP |
| | | PSLPSPAAPEAPELPEPAQP |
| 221 | NCCRP1_1 | GMEADGPASLQELPPSPRSPSPPPSPPPLPSPPSLPSPAAPEA |
| | | PELPEPAQPSEAHARQLLL |
| 222 | NCCRP1_2 | PASLQELPPSPRSPSPPPSPPPLPSPPSLPSPAAPEAPELPEPA |
| | | QPSEAHARQLLLEEWGPL |
| 223 | EMILIN2 | RGLPRGVDGQTGSGTVPGAEGFAGAPGYPKSPPVASPGAP |
| | | VPSLVSFSAGLTQKPFPSDGGV |
| 224 | LMOD1 | GNTDTKKDDEKVKKNEPLHEKEAKDDSKTKTPEKQTPSG |
| | | PTKPSEGPAKVEEEAAPSIFDEP |
| 225 | MYBPC2 | GKDAPKGAPKEAPPKEAPAEAPKEAPPEDQSPTAEEPTGV |
| | | FLKKPDSVSVETGKDAVVVAKV |
| 226 | MAGI2_0 | TSAPSSEKQSPMAQQSPLAQQSPLAQPSPATPNSPIAQPAPP |
| | | QPLQLQGHENSYRSEVKARQ |
| 227 | MAGI2_1 | DEPAPWSSPAAAAPGLPEVGVSLDDGLAPFSPSHPAPPSDP |
| | | SHQISPGPTWDIKREHDVRKP |
| 228 | MAGI2_2 | LPEVGVSLDDGLAPFSPSHPAPPSDPSHQISPGPTWDIKREH |
| | | DVRKPKELSACGQKKQRLGE |
| 229 | RTN2 | LDLRLRLAQPSSPEVLTPQLSPGSGTPQAGTPSPSRSRDSNS |
| | | GPEEPLLEEEEKQWGPLERE |
| 230 | TP53BP2 | QGKPGSPEPETEPVSSVQENHENERIPRPLSPTKLLPFLSNP |
| | | YRNQSDADLEALRKKLSNAP |
| 231 | HCN1_0 | PPVYTATSLSHSNLHSPSPSTQTPQPSAILSPCSYTTAVCSPP |
| | | VQSPLAARTFHYASPTASQ |
| 232 | HCN1_1 | PTASQLSLMQQQPQQQVQQSQPPQTQPQQPSPQPQTPGSS |
| | | TPKNEVHKSTQALHNTNLTREV |
| 233 | HCN1_2 | LSLMQQQPQQQVQQSQPPQTQPQQPSPQPQTPGSSTPKNE |
| | | VHKSTQALHNTNLTREVRPLSA |
| 234 | HCN1_3 | QQPQQQVQQSQPPQTQPQQPSPQPQTPGSSTPKNEVHKST |
| | | QALHNTNLTREVRPLSASQPSL |
| 235 | TRIM10 | NERPARELLTDIRSTLIRCETRKCRKPVAVSPELGQRIRDFP |
| | | QQALPLQREMKMFLEKLCFE |
| 236 | KCNH4 | VSQLSRELRHIMGLLQARLGPPGHPAGSAWTPDPPCPQLR |
| | | PPCLSPCASRPPPSLQDTTLAE |
| 237 | MEGF9 | APTTLSTTTGPAPTTPVATTVPAPTTPRTPTPDLPSSSNSSV |
| | | LPTPPATEAPSSPPPEYVCN |
| 238 | COL24A1 | LPGIRGGPGRTGLAGAPGPPGVKGSSGLPGSPGIQGPKGEQ |
| | | GLPGQPGIQGKRGHRGAQGDQ |
| 239 | PLA2G3 | GTVPLARLQPRTFYNASWSSRATSPTPSSRSPAPPKPRQKQ |
| | | HLRKGPPHQKGSKRPSKANTT |
| 240 | FRS3 | DETPLQKPTSTRAAIRSHGSFPVPLTRRRGSPRVFNFDFRRP |
| | | GPEPPRQLNYIQVELKGWGG |
| 241 | NYNRIN | PSLSEEILRCLSLHDPPDGALDIDLLPGAASPYLGIPWDGK |
| | | APCQQVLAHLAQLTIPSNFTA |
| 242 | MBD6_0 | NAPSYNWGAALRSSLVPSDLGSPPAPHASSSPPSDPPLFHC |
| | | SDALTPPPLPPSNNLPAHPGP |
| 243 | MBD6_1 | VPSDLGSPPAPHASSSPPSDPPLFHCSDALTPPPLPPSNNLP |
| | | AHPGPASQPPVSSATMHLPL |
| 244 | MBD6_2 | ASHSSSLRPSQRRPRRPPTVFRLLEGRGPQTPRRSRPRAPAP |
| | | VPQPFSLPEPSQPILPSVLS |
| 245 | MBD6_3 | PSLPGTTSGSLSSVPGAPAPPAASKAPVVPSPVLQSPSEGLG |
| | | MGAGPACPLPPLAGGEAFPF |
| 246 | MBD6_4 | TTSGSLSSVPGAPAPPAASKAPVVPSPVLQSPSEGLGMGA |
| | | GPACPLPPLAGGEAFPFPSPEQ |
| 247 | MBD6_5 | APCLPPESPASALEPEPARPPLSALAPPHGSPDPPVPELLTG |
| | | RGSGKRGRRGGGGLRGINGE |
| 248 | PRR35_0 | LYNHMKYSLCKDSLSLLLDSPDWACRRGSTTPRPHAPTPD |
| | | RPGESDPGRQPQGARPTGAAPA |
| 249 | PRR35_1 | AAAHVPFLASASPLLPPATAFPAVQPPQRPTPAPRLYYPLL |
| | | LEHTLGLPAGKAALAKAPVSP |
| 250 | PRR35_2 | SLTRFCSRSSLPTGSSVMLWPEDGDPGGPETPGPEGPLPLQ |
| | | PRGPVPGSPEHVGEDLTRALG |
| 251 | CACNA1D | LMQQQIMAVAGLDSSKAQKYSPSHSTRSWATPPATPPYR |
| | | DWTPCYTPLIQVEQSEALDQVNG |
| 252 | ORAI3 | FSTALGTFLFLAEVVLVGWVKFVPIGAPLDTPTPMVPTSR |
| | | VPGTLAPVATSLSPASNLPRSS |
| 253 | FOXE3 | GPPLPFPYAPYAPAPGPALLVPPPSAGPGPSPPARLFSVDSL |
| | | VNLQPELAGLGAPEPPCCAA |
| 254 | POM121C_0 | SSPAAPAASSASPMFKPIFTAPPKSEKEGLTPPGPSVSATAP |
| | | SSSSLPTTTSTTAPTFQPVF |
| 255 | POM121C_1 | AADFSGFGSTLATSAPATSSQPTLTFSNTSTPTFNIPFGSSA |
| | | KSPLPSYPGANPQPAFGAAE |
| 256 | MMP24 | LQGIQKIYGPPAEPLEPTRPLPTLPVRRIHSPSERKHERQPR |
| | | PPRPPLGDRPSTPGTKPNIC |
| 257 | GPR162 | PPRGPGFFREEITTFIDETPLPSPTASPGHSPRRPRPLGLSPR |
| | | RLSLGSPESRAVGLPLGLS |
| 258 | ZMIZ1_0 | GNPMANANNPMNPGGNPMASGMTTSNPGLNSPQFAGQQ |
| | | QQFSAKAGPAQPYIQQSMYGRPNY |
| 259 | ZMIZ1_1 | YSNYSQGNVNRPPRPVPVANYPHSPVPGNPTPPMTPGSSIP |
| | | PYLSPSQDVKPPFPPDIKPNM |
| 260 | ADAMTSL5 | FQARVQALGWPLRQPQPRGVEPQPPAAPAVTPAQTPTLAP |
| | | DPCPPCPDTRGRAHRLLHYCGS |
| 261 | PPP2R3A | AVLIQQTPEVIKIQNKPEKKPGTPLPPPATSPSSPRPLSPVPH |
| | | VNNVVNAPLSINIPRFYFP |
| 262 | PCDH8 | SPEEAARGAGPRPNMFDVLTFPGTGKAPFGSPAADAPPPA |
| | | VAAAEVPGSEGGSATGESACHF |
| 263 | MMP25 | LYGKAPQTPYDKPTRKPLAPPPQPPASPTHSPSFPIPDRCEG |
| | | NFDAIANIRGETFFFKGPWF |
| 264 | COL5A3_0 | GRKKNKEIWTSSPPPDSAENQTSTDIPKTETPAPNLPPTPTP |
| | | LVVTSTVTTGLNATILERSL |
| 265 | COL5A3_1 | SSPPPDSAENQTSTDIPKTETPAPNLPPTPTPLVVTSTVTTG |
| | | LNATILERSLDPDSGTELGT |
| 266 | COL5A3_2 | FPGPKGGPGDPGPTGLKGDKGPPGPVGANGSPGERGPLGP |
| | | AGGIGLPGQSGSEGPVGPAGKK |
| 267 | COL5A3_3 | DPGPPGPIGSLGHPGPPGVAGPLGQKGSKGSPGSMGPRGD |
| | | TGPAGPPGPPGAPAELHGLRRR |
| 268 | SOX7 | PLHCSHPLGSLALGQSPGVSMMSPVPGCPPSPAYYSPATY |
| | | HPLHSNLQAHLGQLSPPPEHPG |
| 269 | SEZ6L | IVASEEASEVPLWLDRKESAVPTTPAPLQISPFTSQPYVAH |
| | | TLPQRPEPGEPGPDMAQEAPQ |
| 270 | VGF | GSQQGPEEEAAEALLTETVRSQTHSLPAPESPEPAAPPRPQ |
| | | TPENGPEASDPSEELEALASL |
| 271 | PRR30 | LSPHQGLPPSQPPFSSTQSRRPSSPPPASPSPGFQFGSCDSNS |
| | | DFAPHPYSPSLPSSPTFFH |
| 272 | SOBP | ASTTVSPSDTANCSVTKIPTPVPKSIPISETPNIPPVSVQPPAS |
| | | IGPPLGVPPRSPPMVMTN |
| 273 | INO80B_0 | LKLKIKLGGQVLGTKSVPTFTVIPEGPRSPSPLMVVDNEEE |
| | | PMEGVPLEQYRAWLDEDSNLS |
| 274 | INO80B_1 | PMVRYCSGAQGSTLSFPPGVPAPTAVSQRPSPSGPPPRCSV |
| | | PGCPHPRRYACSRTGQALCSL |
| 275 | POU5F1_0 | YAQREDFEAAGSPFSGGPVSFPLAPGPHFGTPGYGSPHFTA |
| | | LYSSVPFPEGEAFPPVSVTTL |
| 276 | POU5F1_1 | DFEAAGSPFSGGPVSFPLAPGPHFGTPGYGSPHFTALYSSV |
| | | PFPEGEAFPPVSVTTLGSPMH |
| 277 | ERICH6 | FPDVRPRLASIVSPSLTSTFVPSQSATSTETPSASPPSSTSSH |
| | | KSFPKIFQTFRKDMSEMSI |
| 278 | B4GALNT1 | LACASLGLLYASTRDAPGLRLPLAPWAPPQSPRRPELPDL |
| | | APEPRYAHIPVRIKEQVVGLLA |
| 279 | ABRA | ANENSIRQAQEPTGWLPGGTQDSPQAPKPITPPTSHQKAQS |
| | | APKSPPRLPEGHGDGQSSEKA |
| 280 | PLCH2 | TGSKGVADDVVPPGPGPAPEAPAQEGPGSGSPRDTRPLST |
| | | QRPLPPLCSLETIAEEPAPGPG |
| 281 | STAC2_0 | LKCPTEVLLTPPTPLPPPSPPPTASDRGLATPSPSPCPVPRPL |
| | | AALKPVRLHSFQEHVFKRA |
| 282 | STAC2_1 | IRSSEEGPGDSASPVFTAPAESEGPGPEEKSPGQQLPKATLR |
| | | KDVGPMYSYVALYKFLPQEN |
| 283 | MAPK8IP2 | EEEEEEEGDGEGQEGGDPGSEAPAPGPLIPSPSVEEPHKHR |
| | | PTTLRLTTLGAQDSLNNNGGF |
| 284 | PARM1 | TNHSSTVTSTQPTGAPTAPESPTEESSSDHTPTSHATAEPVP |
| | | QEKTPPTTVSGKVMCELIDM |
| 285 | MMP28 | QSLYGKPLGGSVAVQLPGKLFTDFETWDSYSPQGRRPETQ |
| | | GPKYCHSSFDAITVDRQQQLYI |
| 286 | SPEF2 | EGKGKKGETALKRKGSPKGKSSGGKVPVKKSPADSTDTS |
| | | PVAIVPQPPKPGSEEWVYVNEPV |
| 287 | CMYA5 | EGKKPSPEVKIPTQRKPISSIHAREPQSPESPEVTQNPPTQPK |
| | | VAKPDLPEEKGKKGISSFK |
| 288 | VPS37C_0 | PVRPVPQGTPPVVEEQPQPPLAMPPYPLPYSPSPSLPVGPT |
| | | AHGALPPAPFPVVSQPSFYSG |
| 289 | VPS37C_1 | RPVPQGTPPVVEEQPQPPLAMPPYPLPYSPSPSLPVGPTAH |
| | | GALPPAPFPVVSQPSFYSGPL |
| 290 | TMEM200B | LRQGVLRAQALRPPDGPGWDCALLPSPGPRSPRAVGCAEP |
| | | EIWDPSPRRGTSPVPSVRSLRS |
| 291 | PAPPA | PCSPSGHWSPREAEGHPDVEQPCKSSVRTWSPNSAVNPHT |
| | | VPPACPEPQGCYLELEFLYPLV |
| 292 | HIVEP3_0 | GKGPGQDRPPLGPTVPYTEALQVFHHPVAQTPLHEKPYLP |
| | | PPVSLFSFQHLVQHEPGQSPEF |
| 293 | HIVEP3_1 | SLFSFQHLVQHEPGQSPEFFSTQAMSSLLSSPYSMPPLPPSL |
| | | FQAPPLPLQPTVLHPGQLHL |
| 294 | HIVEP3_2 | DYPKERERTGGGPGRPPDWTPHGTGAPAEPTPTHSPCTPP |
| | | DTLPRPPQGRRAAQSWSPRLES |
| 295 | SEC31B_0 | TLHSKETSSYRLGSQPSHQVPTPSPRPRVFTPQSSPAMPLA |
| | | PSHPSPYQGPRTQNISDYRAP |
| 296 | SEC31B_1 | PSHQVPTPSPRPRVFTPQSSPAMPLAPSHPSPYQGPRTQNIS |
| | | DYRAPGPQAIQPLPLSPGVR |
| 297 | NYAP1 | PQQPHALPPHAHRRPASALPSRRDGTPTKTTPCEIPPPFPNL |
| | | LQHRPPLLAFPQAKSASRTP |
| 298 | CAMTA2_0 | AGGRRGNCFFIQDDDSGEELKGHGAAPPIPSPPPSPPPSPAP |
| | | LEPSSRVGRGEALFGGPVGA |
| 299 | CAMTA2_1 | VAHSRGHVRLARCLEELQRQEPSVEPPFALSPPSSSPDTGL |
| | | SSVSSPSELSDGTFSVTSAYS |
| 300 | CAMTA2_2 | GHVRLARCLEELQRQEPSVEPPFALSPPSSSPDTGLSSVSSP |
| | | SELSDGTFSVTSAYSSAPDG |
| 301 | SYNPO2L_0 | AYYGETDSDADGPATQEKPRRPRRRGPTRPTPPGAPPDEV |
| | | YLSDSPAEPAPTIPGPPSQGDS |
| 302 | SYNPO2L_1 | TQEKPRRPRRRGPTRPTPPGAPPDEVYLSDSPAEPAPTIPGP |
| | | PSQGDSRVSSPSWEDGAALQ |
| 303 | SYNPO2L_2 | GEGLQSPPRAQSAPPEAAVLPPSPLPAPVASPRPFQPGGGA |
| | | PTPAPSIFNRSARPFTPGLQG |
| 304 | SYNPO2L_3 | ACNFMQPVGARSYKTLPHVTPKTPPPMAPKTPPPMTPKTP |
| | | PPVAPKPPSRGLLDGLVNGAAS |
| 305 | SYNPO2L_4 | QPVGARSYKTLPHVTPKTPPPMAPKTPPPMTPKTPPPVAP |
| | | KPPSRGLLDGLVNGAASSAGIP |
| 306 | SYNPO2L_5 | FAKRQSRADRYVVEGTPGPGLGPRPRSPSPTPSLPPSWKYS |
| | | PNIRAPPPIAYNPLLSPFFPQ |
| 307 | MUC5B_0 | CCEYVPCGPSPAPGTSPQPSLSASTEPAVPTPTQTTATEKTT |
| | | LWVTPSIRSTAALTSQTGSS |
| 308 | MUC5B_1 | TPGTAHTTKVPTTTTTGFTATPSSSPGTALTPPVWISTTTTP |
| | | TTTTPTTSGSTVTPSSIPGT |
| 309 | MUC5B_2 | ASCKDMAKTWLVPDSRKDGCWAPTGTPPTASPAAPVSST |
| | | PTPTPCPPQPLCDLMLSQVFAEC |
| 310 | MUC5B_3 | LVPDSRKDGCWAPTGTPPTASPAAPVSSTPTPTPCPPQPLC |
| | | DLMLSQVFAECHNLVPPGPFF |
| 311 | SCML4 | KIPKKRGRKPGYKIKSRVLMTPLALSPPRSTPEPDLSSIPQD |
| | | AATVPSLAAPQALTVCLYIN |
| 312 | RIN3 | PPVLPLQPCSPAQPPVLPALAPAPACPLPTSPPVPAPHVTPH |
| | | APGPPDHPNQPPMMTCERLP |
| 313 | RBBP8NL | QRISNQLHGTIAVVRPGSQACPADRGPANGTPPPLPARSSP |
| | | PSPAYERGLSLDSFLRASRPS |
| 314 | ADGRG2_0 | VPKATSFAEPPDYSPVTHNVPSPIGEIQPLSPQPSAPIASSPA |
| | | IDMPPQSETISSPMPQTHV |
| 315 | ADGRG2_1 | PDYSPVTHNVPSPIGEIQPLSPQPSAPIASSPAIDMPPQSETIS |
| | | SPMPQTHVSGTPPPVKAS |
| 316 | ADGRG2_2 | SAPIASSPAIDMPPQSETISSPMPQTHVSGTPPPVKASFSSPT |
| | | VSAPANVNTTSAPPVQTDI |
| 317 | ADGRG2_3 | DMPPQSETISSPMPQTHVSGTPPPVKASFSSPTVSAPANVN |
| | | TTSAPPVQTDIVNTSSISDLE |
| 318 | C9orf131 | SSLSTPLPEPHIDLELVWRNVQQREVPQGPSPLAVDPLHPV |
| | | PQPPTLAEAVKIERTHPGLPK |
| 319 | SLC30A6 | VAANVLNFSDHHVIPMPLLKGTDDLNPVTSTPAKPSSPPPE |
| | | FSFNTPGKNVNPVILLNTQTR |
| 320 | HEYL | FFHSCPGLPALSNQLAILGRVPSPVLPGVSSPAYPIPALRTA |
| | | PLRRATGIILPARRNVLPSR |
| 321 | SPPL2B | WTGSGFAKVLPPSPWAPAPADGPQPPKDSATPLSPQPPSEE |
| | | PATSPWPAEQSPKSRTSEEMG |
| 322 | CACNB1 | EAERQALAQLEKAKTKPVAFAVRTNVGYNPSPGDEVPVQ |
| | | GVAITFEPKDFLHIKEKYNNDWW |
| 323 | PRR16 | YNIKNREVHLHSEPVHPPGKIPHQGPPLPPTPHLPPFPLENG |
| | | GMGISHSNSFPPIRPATVPP |
| 324 | TRABD2B | HTPAGQAIHSPAPQSPAPSPEGTSTSPAPVTPAAAVPEAPS |
| | | VTPTAPPEDEDPALSPHLLLP |
| 325 | PRR18 | SSWPSATLKRPPARRGPGLDRTQPPAPPGVSPQALPSRAR |
| | | APATCAPPRPAGSGHSPARTTY |
| 326 | UBALD1 | ATSSSAASSWPTAASPPGGPQHHQPQPPLWTPTPPSPASD |
| | | WPPLAPQQATSEPRAHPAMEAE |
| 327 | RTL3 | YDLLRKSSEAKEPQKLPEHMNPPAAWEAQKTPEFKEPQK |
| | | PPEPQDLLPWEPPAAWELQEAPA |
| 328 | RNF149_0 | EMPAPESPPGRDPAANLSLALPDDDGSDDSSPPSASPAESE |
| | | PQCDPSFKGDAGENTALLEAG |
| 329 | RNF149_1 | ESPPGRDPAANLSLALPDDDGSDDSSPPSASPAESEPQCDP |
| | | SFKGDAGENTALLEAGRSDSR |
| 330 | PTPRQ | GYGNASNWISTKTLPGPPDGPPENVHVVATSPFSISISWSE |
| | | PAVITGPTCYLIDVKSVDNDE |
| 331 | PLSCR3 | YPEPALHPGPGQAPVPAQVPAPAPGFALFPSPGPVALGSA |
| | | APFLPLPGVPSGLEFLVQIDQI |
| 332 | HAVCR1 | TTTSVPVTTTVSTFVPPMPLPRQNHEPVATSPSSPQPAETH |
| | | PTTLQGAIRREPTSSPLYSYT |
| 333 | DNAJC30 | RRKYDRGLLSDEDLRGPGVRPSRTPAPDPGSPRTPPPTSRT |
| | | HDGSRASPGANRTMFNFDAFY |
| 334 | LPO | RKPALGAANRALARWLPAEYEDGLSLPFGWTPGKTRNGF |
| | | PLPLAREVSNKIVGYLNEEGVLD |
| 335 | PYGO1 | SSNPYLGPGYPGFGGYSTFRMPPHVPPRMSSPYCGPYSLR |
| | | NQPHPFPQNPLGMGFNRPHAFN |
| 336 | ADGRG4 | NYATSLNTPVSYPPWTPSSATLPSLTSFVYSPHSTEAEISTP |
| | | KTSPPPTSQMVEFPVLGTRM |
| 337 | SYN3 | PGSSLFSSLSSAMKQAPQATSGLMEPPGPSTPIVQRPRILLV |
| | | IDDAHTDWSKYFHGKKVNGE |
| 338 | MAP3K13 | SGMQTKRPDLLRSEGIPTTEVAPTASPLSGSPKMSTSSSKS |
| | | RYRSKPRHRRGNSRGSHSDFA |
| 339 | SFTPA2 | PGSHGLPGRDGRDGVKGDPGPPGPMGPPGETPCPPGNNG |
| | | LPGAPGVPGERGEKGEAGERGPP |
| 340 | HECW1 | SSEKDGLSEVDTVAADPSALEEDREEPEGATPGTAHPGHS |
| | | GGHFPSLANGAAQDGDTHPSTG |
| 341 | CELF3 | ITPSSGTSTPPAIAATPVSAIPAALGVNGYSPVPTQPTGQPA |
| | | PDALYPNGVHPYPAQSPAAP |
| 342 | INAFM1 | AAVLLAVYYGLIWVPTRSPAAPAGPQPSAPSPPCAARPGV |
| | | PPVPAPAAASLSCLLGVPGGPR |
| 343 | CDX1 | KDDWAAAYGPGPAAPAASPASLAFGPPPDFSPVPAPPGPG |
| | | PGLLAQPLGGPGTPSSPGAQRP |
| 344 | TEX13D | SRSHSQGEGSERSQRMPLPGDSGCHNPLSESPQGTAPLGSS |
| | | GCHSQEEGTEGPQGMDPLGNR |
| 345 | NDST2 | FLQCWTRLRLQTLPPVPLAQKYFELFPQERSPLWQNPCDD |
| | | KRHKDIWSKEKTCDRLPKFLIV |
| 346 | SPATA31E1 | DPLGDVCKPVPAKAHQPHGKCMQDPSPASLSPPAPPAPLA |
| | | STLSPGPMTFSEPFGPHSTLSA |
| 347 | SPATC1 | LAPQVATSYTPSSTTHIAQGAPHPPSRMHNSPTQNLPVPHC |
| | | PPHNAHSPPRTSSSPASVNDS |
| 348 | SIGLEC12_0 | SARPAVGVGDTGMEDANAVRGSASQGPLIESPADDSPPH |
| | | HAPPALATPSPEEGEIQYASLSF |
| 349 | SIGLEC12_1 | VGVGDTGMEDANAVRGSASQGPLIESPADDSPPHHAPPAL |
| | | ATPSPEEGEIQYASLSFHKARP |
| 350 | SOWAHA | SVEESGLGLGLGPGRSPHLRRLSRAGPRLLSPDAEELPAAP |
| | | PPSAVPLEPSEHEWLVRTAGG |
| 351 | RAPGEF5 | VGSVKMQPPCESPALAAAAAVVAADGPLRRSPSAREPER |
| | | EQPPASLRPRLRDLPALLRSGLT |
| 352 | ADRB1 | RVFREAQKQVKKIDSCERRFLGGPARPPSPSPSPVPAPAPP |
| | | PGPPRPAAAAATAPLANGRAG |
| 353 | CNGB1 | ATGAASDPAPPGRPQEMGPKLQARETPSLPTPIPLQPKEEP |
| | | KEAPAPEPQPGSQAQTSSLPP |
| 354 | PROB1_0 | DRTVQRARSPPFECRIPSEVPSRAVRPRSPSPPRQTPNGAV |
| | | RGPRCPSPQNLSPWDRTTRRV |
| 355 | PROB1_1 | RARSPPFECRIPSEVPSRAVRPRSPSPPRQTPNGAVRGPRCP |
| | | SPQNLSPWDRTTRRVSSPLF |
| 356 | PROB1_2 | QAPLPREPLALAGRTAPAQPRAASAPPTDRSPQSPSQGAR |
| | | RQPGAAPLGKVLVDPESGRYYF |
| 357 | SPATA31D1 | LADLFSPSPLRDPLPPQPVSPLDSKFPIDHSPPQQLPFPLLPP |
| | | HHIERVESSLQPEASLSLN |
| 358 | ARHGEF18 | RSLSPILPGRHSPAPPPDPGFPAPSPPPADSPSEGFSLKAGGT |
| | | ALLPGPPAPSPLPATPLSA |
| 359 | ALPK1 | SLQEPNNDNLEPSQNQPQQQMPLTPFSPHNTPGIFLAPGAG |
| | | LLEGAPEGIQEVRNMGPRNTS |
| 360 | PRICKLE1 | EYAWVPPGLRPEQIQLYFACLPEEKVPYVNSPGEKHRIKQ |
| | | LLYQLPPHDNEVRYCQSLSEEE |
| 361 | B4GALNT3 | TASFPGRTSHIPVQQPEKRKQKPSPEPSQDSPHSDKWPPGH |
| | | PVKNLPQMRGPRPRPAGDSPR |
| 362 | KRTAP10-2 | QVDDCPESCCELPCGTPSCCAPAPCLTLVCTPVSCVSSPCC |
| | | QAACEPSACQSGCTSSCTPSC |
| 363 | PRDM12 | CQSAYSQLAGLRAHQKSARHRPPSTALQAHSPALPAPHA |
| | | HAPALAAAAAAAAAAAAHHLPAM |
| 364 | POU6F2_0 | ELRGEDKAATSDSELNEPLLAPVESNDSEDTPSKLFGARG |
| | | NPALSDPGTPDQHQASQTHPPF |
| 365 | POU6F2_1 | QQQQPPPSTNQHPQPAPQAPSQSQQQPLQPTPPQQPPPASQ |
| | | QPPAPTSQLQQAPQPQQHQPH |
| 366 | POU6F2_2 | QQHQPHSHSQNQNQPSPTQQSSSPPQKPSQSPGHGLPSPLT |
| | | PPNPLQLVNNPLASQAAAAAA |
| 367 | POU6F2_3 | NQNQPSPTQQSSSPPQKPSQSPGHGLPSPLTPPNPLQLVNN |
| | | PLASQAAAAAAAMSSIASSQA |
| 368 | LDB3_0 | KIKSASYNLSLTLQKSKRPIPISTTAPPVQTPLPVIPHQKDPA |
| | | LDTNGSLVAPSPSPEARAS |
| 369 | LDB3_1 | AAPAPKPRVVTTASIRPSVYQPVPASTYSPSPGANYSPTPY |
| | | TPSPAPAYTPSPAPAYTPSPV |
| 370 | LDB3_2 | VVTTASIRPSVYQPVPASTYSPSPGANYSPTPYTPSPAPAYT |
| | | PSPAPAYTPSPVPTYTPSPA |
| 371 | LDB3_3 | SIRPSVYQPVPASTYSPSPGANYSPTPYTPSPAPAYTPSPAP |
| | | AYTPSPVPTYTPSPAPAYTP |
| 372 | LDB3_4 | PVPASTYSPSPGANYSPTPYTPSPAPAYTPSPAPAYTPSPVP |
| | | TYTPSPAPAYTPSPAPNYNP |
| 373 | KIAA1549L | SDIPPLLPLPPSSSLAPDSPHSIISEPAEQSPKVLLVPQTAPA |
| | | DPSLGQNIANPLIPFSDEM |
| 374 | FXYD5 | MDIQVPTRAPDAVYTELQPTSPTPTWPADETPQPQTQTQQ |
| | | LEGTDGPLVTDPETHKSTKAAH |
| 375 | HGFAC | CTSEGSAHRKWCATTHNYDRDRAWGYCVEATPPPGGPA |
| | | ALDPCASGPCLNGGSCSNTQDPQS |
| 376 | KCNH6_0 | KPMPQGHASYILEAPASNDLALVPIASETTSPGPRLPQGFL |
| | | PPAQTPSYGDLDDCSPKHRNS |
| 377 | KCNH6_1 | ASNDLALVPIASETTSPGPRLPQGFLPPAQTPSYGDLDDCS |
| | | PKHRNSSPRMPHLAVATDKTL |
| 378 | KCNH6_2 | ASETTSPGPRLPQGFLPPAQTPSYGDLDDCSPKHRNSSPRM |
| | | PHLAVATDKTLAPSSEQEQPE |
| 379 | ADAM19_0 | GCGKKCNGHGVCNNNQNCHCLPGWAPPFCNTPGHGGSI |
| | | DSGPMPPESVGPVVAGVLVAILVL |
| 380 | ADAM19_1 | PFRVSQNSGTGHANPTFKLQTPQGKRKVINTPEILRKPSQP |
| | | PPRPPPDYLRGGSPPAPLPAH |
| 381 | ESYT3 | KKSPATIFLTVPGPHSPGPIKSPRPMKCPASPFAWPPKRLAP |
| | | SMSSLNSLASSCFDLADISL |
| 382 | SHANK1_0 | RSGRGRKGPLVKQTKVEGEPQKGGGLPPAPSPTSPASPQP |
| | | PPAVAAPSEKNSIPIPTIIIKA |
| 383 | SHANK1_1 | RGRKGPLVKQTKVEGEPQKGGGLPPAPSPTSPASPQPPPA |
| | | VAAPSEKNSIPIPTIIIKAPST |
| 384 | SHANK1_2 | PTQPEPTGGGGGGGSSPSPAPAMSPVPPSPSPVPTPASPSGP |
| | | ATLDFTSQFGAALVGAARRE |
| 385 | SHANK1_3 | PVTSGRGPPSEDGPGVPPPSPRRSVPPSPTSPRASEENGLPL |
| | | LVLPPPAPSVDVEDGEFLFV |
| 386 | SHANK1_4 | PSVDVEDGEFLFVEPLPPPLEFSNSFEKPESPLTPGPPHPLPD |
| | | TPAPATPLPPVPPPAVAAA |
| 387 | SHANK1_5 | DVEDGEFLFVEPLPPPLEFSNSFEKPESPLTPGPPHPLPDTP |
| | | APATPLPPVPPPAVAAAPPT |
| 388 | SHANK1_6 | EPLPPPLEFSNSFEKPESPLTPGPPHPLPDTPAPATPLPPVPP |
| | | PAVAAAPPTLDSTASSLTS |
| 389 | SHANK1_7 | PLEFSNSFEKPESPLTPGPPHPLPDTPAPATPLPPVPPPAVA |
| | | AAPPTLDSTASSLTSYDSEV |
| 390 | EMID1 | VSELTERLKVLEAKMTMLTVIEQPVPPTPATPEDPAPLWG |
| | | PPPAQGSPGDGGLQDQVGAWGL |
| 391 | MYOZ3 | ELHIFPASPGASLGGPEGAHPAAAPAGCVPSPSALAPGYAE |
| | | PLKGVPPEKFNHTAISKGYRC |
| 392 | DAB1_0 | PTVAGQFPPAAFMPTQTVMPLPAAMFQGPLTPLATVPGTS |
| | | DSTRSSPQTDKPRQKMGKETFK |
| 393 | DAB1_1 | QTVMPLPAAMFQGPLTPLATVPGTSDSTRSSPQTDKPRQK |
| | | MGKETFKDFQMAQPPPVPSRKP |
| 394 | DAB1_2 | YFNKVGVAQDTDDCDDFDISQLNLTPVTSTTPSTNSPPTPA |
| | | PRQSSPSKSSASHASDPTTDD |
| 395 | DAB1_3 | GVAQDTDDCDDFDISQLNLTPVTSTTPSTNSPPTPAPRQSS |
| | | PSKSSASHASDPTTDDIFEEG |
| 396 | DAB1_4 | DFDISQLNLTPVTSTTPSTNSPPTPAPRQSSPSKSSASHASDP |
| | | TTDDIFEEGFESPSKSEEQ |
| 397 | VEGFB | SAVKPDRAATPHHRPQPRSVPGWDSAPGAPSPADITHPTP |
| | | APGPSAHAAPSTTSALTPGPAA |
| 398 | TOX2_0 | PSFPLSPTLHQQLSLPPHAQGALLSPPVSMSPAPQPPVLPTP |
| | | MALQVQLAMSPSPPGPQDFP |
| 399 | TOX2_1 | QQLSLPPHAQGALLSPPVSMSPAPQPPVLPTPMALQVQLA |
| | | MSPSPPGPQDFPHISEFPSSSG |
| 400 | MAP3K12 | GLLKPHPSRGLLHGNTMEKLIKKRNVPQKLSPHSKRPDIL |
| | | KTESLLPKLDAALSGVGLPGCP |
| 401 | NLGN1 | EILGPVIQFLGVPYAAPPTGERRFQPPEPPSPWSDIRNATQF |
| | | APVCPQNIIDGRLPEVMLPV |
| 402 | POM121_0 | SSPAAPAASSAPPMFKPIFTAPPKSEKEGPTPPGPSVTATAP |
| | | SSSSLPTTTSTTAPTFQPVF |
| 403 | POM121_1 | AADFSGFGSTLATSAPATSSQPTLTFSNTSTPTFNIPFGSSA |
| | | KSPLPSYPGANPQPAFGAAE |
| 404 | PCDH15_0 | LGPMFLPCVLVPNTRDCRPLTYQAAIPELRTPEELNPIIVTP |
| | | PIQAIDQDRNIQPPSDRPGI |
| 405 | PCDH15_1 | VPNTRDCRPLTYQAAIPELRTPEELNPIIVTPPIQAIDQDRNI |
| | | QPPSDRPGILYSILVGTPE |
| 406 | PCDH15_2 | PISPPSPPPAPAPLAPPPDISPFSLFCPPPSPPSIPLPLPPPT |
| | | FFPLSVSTSGPPTPPLLPP |
| 407 | COL4A6 | PCIIPGSYGPSGFPGTPGFPGPKGSRGLPGTPGQPGSSGSKG |
| | | EPGSPGLVHLPELPGFPGPR |
| 408 | MCIDAS_0 | SDSSSMMSPTLASGDFPFSPCDISPFGPCLSPPLDPRALQSP |
| | | PLRPPDVPPPEQYWKEVADQ |
| 409 | MCIDAS_1 | LASGDFPFSPCDISPFGPCLSPPLDPRALQSPPLRPPDVPPPE |
| | | QYWKEVADQNQRALGDALV |
| 410 | NEUROD1 | PPYGTMDSSHVFHVKPPPHAYSAALEPFFESPLTDCTSPSF |
| | | DGPLSPPLSINGNFSFKHEPS |
| 411 | SPATA31A5 | SLSASQPPEPSLPLEHPSPEPPALFPHPPHTPDPLACSLPPPK |
| | | GFTAPPLRDSTLITPSHCD |
| 412 | GCM2 | LSSCNYAPEDTGMSVYPEPWGPPVTVTRAASPSGPPPMKI |
| | | AGDCRAIRPTVAIPHEPVSSRT |
| 413 | TOGARAM2 | PSPLPPGQGVLTGLRAPRTRLARGSGPREKTPASLEPKPLA |
| | | SPIRDRPAAAKKPALPFSQSA |
| 414 | COL4A3_0 | GSKGERGRPGKDAMGTPGSPGCAGSPGLPGSPGPPGPPGD |
| | | IVFRKGPPGDHGLPGYLGSPGI |
| 415 | COL4A3_1 | GEPGLQGTQGVPGAPGPPGEAGPRGELSVSTPVPGPPGPP |
| | | GPPGHPGPQGPPGIPGSLGKCG |
| 416 | COL4A3_2 | PHGDLGFKGIKGLLGPPGIRGPPGLPGFPGSPGPMGIRGDQ |
| | | GRDGIPGPAGEKGETGLLRAP |
| 417 | COL4A3_3 | DKGSMGHPGPKGPPGTAGDMGPPGRLGAPGTPGLPGPRG |
| | | DPGFQGFPGVKGEKGNPGFLGSI |
| 418 | GRIN2C | GRRAPPPSPCPTPRSGPSPCLPTPDPPPEPSPTGWGPPDGGR |
| | | AALVRRAPQPPGRPPTPGPP |
| 419 | SOHLH1 | DPGTGASSGTRTPDVKAFLESPWSLDPASASPEPVPHILAS |
| | | SRQWDPASCTSLGTDKCEALL |
| 420 | ZNF469_0 | QPAAEELGFHRCFQEPPSSFTSTNYTSPSATPRPPAPGPPQS |
| | | RGTSPLQPGSYPEYQASGAD |
| 421 | ZNF469_1 | QGGSQGALGTAGKTPGPREKLPAVRSSQGGSPALFTYNG |
| | | MTDPGAQPLFFGVAQPQVSPHGT |
| 422 | ZNF469_2 | GDLAACAPSPTSAAHMPCSLGPLPREDPLTSPSRAQGGLG |
| | | GQLPASPSCRDPPGPQQLLACS |
| 423 | ZNF469_3 | PGPARSESVGSFGRAPSAPDKPPRTPRKQATPSRVLPTKPK |
| | | PNSQNKPRPPPSEQRKAEPGH |
| 424 | CCDC80 | VTRSTSRAVTVAARPMTTTAFPTTQRPWTPSPSHRPPTTTE |
| | | VITARRPSVSENLYPPSRKDQ |
| 425 | POU5F1B_0 | YAQREDFEAAGSPFSGGPVSFPPAPGPHFGTPGYGSPHFTA |
| | | LYSSVPFPEGEVFPPVSVITL |
| 426 | POU5F1B_1 | DFEAAGSPFSGGPVSFPPAPGPHFGTPGYGSPHFTALYSSV |
| | | PFPEGEVFPPVSVITLGSPMH |
| 427 | COL4A4 | GRKGESGIGAKGEKGIPGFPGPRGDPGSYGSPGFPGLKGEL |
| | | GLVGDPGLFGLIGPKGDPGNR |
| 428 | SULT1A4 | KCNRAPIYVRVPFLEVNDPGEPSGLETLKDTPPPRLIKSHLP |
| | | LALLPQTLLDQKVKVVYVAR |
| 429 | SULT1A3 | KCNRAPIYVRVPFLEVNDPGEPSGLETLKDTPPPRLIKSHLP |
| | | LALLPQTLLDQKVKVVYVAR |
| 430 | ADGRL1_0 | GPPDPSAGPATSPPLSTTTTARPTPLTSTASPAATTPLRRAP |
| | | LTTHPVGAINQLGPDLPPAT |
| 431 | ADGRL1_1 | SAGPATSPPLSTTTTARPTPLTSTASPAATTPLRRAPLTTHP |
| | | VGAINQLGPDLPPATAPVPS |
| 432 | COL1A2 | ASGPAGVRGPNGDAGRPGEPGLMGPRGLPGSPGNIGPAG |
| | | KEGPVGLPGIDGRPGPIGPAGAR |
| 433 | WIZ_0 | CLIKKEPPAGDLAPALAEDGPPTVAPGPVQSPLPLSPLAGR |
| | | PGKPGAGPAQVPRELSLTPIT |
| 434 | WIZ_1 | EPPAGDLAPALAEDGPPTVAPGPVQSPLPLSPLAGRPGKPG |
| | | AGPAQVPRELSLTPITGAKPS |
| 435 | CBLL2 | DHIQNNSDSGAKKPTPPDYYPECQSQPAVSSPHHIIPQKQH |
| | | YAPPPSPSSPVNHQMPYPPQD |
| 436 | ATXN7_0 | SAVGPTCPATVSSLVKPGLNCPSIPKPTLPSPGQILNGKGLP |
| | | APPTLEKKPEDNSNNRKFLN |
| 437 | ATXN7_1 | KPHTPSLPRPPGCPAQQGGSAPIDPPPVHESPHPPLPATEPA |
| | | SRLSSEEGEGDDKEESVEKL |
| 438 | FLRT2 | MAVRELNMNLLSCPTTTPGLPLFTPAPSTASPTTQPPTLSIP |
| | | NPSRSYTPPTPTTSKLPTIP |
| 439 | GRB10_0 | VRRLQEEDQQFRTSSLPAIPNPFPELCGPGSPPVLTPGSLPP |
| | | SQAAAKQDVKVFSEDGTSKV |
| 440 | GRB10_1 | EEDQQFRTSSLPAIPNPFPELCGPGSPPVLTPGSLPPSQAAA |
| | | KQDVKVFSEDGTSKVVEILA |
| 441 | TNFRSF10C_0 | CTSWDDIQCVEEFGANATVETPAAEETMNTSPGTPAPAAE |
| | | ETMNTSPGTPAPAAEETMTTSP |
| 442 | TNFRSF10C_1 | NATVETPAAEETMNTSPGTPAPAAEETMNTSPGTPAPAAE |
| | | ETMTTSPGTPAPAAEETMTTSP |
| 443 | TNFRSF10C_2 | SPGTPAPAAEETMNTSPGTPAPAAEETMTTSPGTPAPAAEE |
| | | TMTTSPGTPAPAAEETMITSP |
| 444 | TNFRSF10C_3 | SPGTPAPAAEETMTTSPGTPAPAAEETMTTSPGTPAPAAEE |
| | | TMITSPGTPASSHYLSCTIVG |
| 445 | PIK3C2B | SGKPVARSKTMPPQVPPRTYASRYGNRKNATPGKNRRISA |
| | | APVGSRPHTVANGHELFEVSEE |
| 446 | PRPF40B | AGKQQQQLPQTLQPQPPQPQPDPPPVPPGPTPVPTGLLEPE |
| | | PGGSEDCDVLEATQPLEQGFL |
| 447 | OLFML2B | SVLQPSPQVPATTVAHTATQQPAAPAPPAVSPREALMEA |
| | | MHTVPVPPTTVRTDSLGKDAPAG |
| 448 | GRIN2D | RYYGPIEPQGLGLGLGEARAAPRGAAGRPLSPPAAQPPQK |
| | | PPPSYFAIVRDKEPAEPPAGAF |
| 449 | GFY | LLAGLRSKAAPSAPLPLGCGFPDMAHPSETSPLKGASENS |
| | | KRDRLNPEFPGTPYPEPSKLPH |
| 450 | TBXT | NHRWKYVNGEWVPGGKPEPQAPSCVYIHPDSPNFGAHW |
| | | MKAPVSFSKVKLTNKLNGGGQIML |
| 451 | ARHGAP44 | GTACAGTQPGAQPGAQPGASPSPSQPPADQSPHTLRKVSK |
| | | KLAPIPPKVPFGQPGAMADQSA |
| 452 | ASCL2 | VRNALAGGLRPQAVRPSAPRGPPGTTPVAASPSRASSSPG |
| | | RGGSSEPGSPRSAYSSDDSGCE |
| 453 | DOK3 | AIARQRERLPELTRPQPCPLPRATSLPSLDTPGELREMPPGP |
| | | EPPTSRKMHLAEPGPQSLPL |
| 454 | DLX5 | VFDRRVPSIRSGDFQAPFQTSAAMHHPSQESPTLPESSATD |
| | | SDYYSPTGGAPHGYCSPTSAS |
| 455 | MAP3K14 | SLAHAGVALAKPLPRTPEQESCTIPVQEDESPLGAPYVRNT |
| | | PQFTKPLKEPGLGQLCFKQLG |
| 456 | PAX9 | LAQQGHYDSYKQHQPTPQPALPYNHIYSYPSPITAAAAKV |
| | | PTPPGVPAIPGSVAMPRTWPSS |
| 457 | ARHGEF15 | DSQTSPDSPSSTPTPSPVSRRSASPEPAPRSPVPPPKPSGSPC |
| | | TPLLPMAGVLAQNGSASAP |
| 458 | NEDD9 | TKPAGKDLHVKYNCDIPGAAEPVARRHQSLSPNHPPPQLG |
| | | QSVGSQNDAYDVPRGVQFLEPP |
| 459 | MUC7_0 | NTSSSVATLAPVNSPAPQDTTAAPPTPSATTPAPPSSSAPPE |
| | | TTAAPPTPSATTQAPPSSSA |
| 460 | MUC7_1 | ETTAAPPTPSATTQAPPSSSAPPETTAAPPTPPATTPAPPSSS |
| | | APPETTAAPPTPSATTPAP |
| 461 | MUC7_2 | PPTPSATTQAPPSSSAPPETTAAPPTPPATTPAPPSSSAPPET |
| | | TAAPPTPSATTPAPLSSSA |
| 462 | MUC7_3 | ETTAAPPTPPATTPAPPSSSAPPETTAAPPTPSATTPAPLSSS |
| | | APPETTAVPPTPSATTLDP |
| 463 | MUC7_4 | PPTPPATTPAPPSSSAPPETTAAPPTPSATTPAPLSSSAPPET |
| | | TAVPPTPSATTLDPSSASA |
| 464 | MUC7_5 | PPTPSATTLDPSSASAPPETTAAPPTPSATTPAPPSSPAPQET |
| | | TAAPITTPNSSPTTLAPDT |
| 465 | RCAN2 | KLYFAQVQTPETDGDKLHLAPPQPAKQFLISPPSSPPVGW |
| | | QPINDATPVLNYDLLYAVAKLG |
| 466 | MXRA8 | HLHHHYCGLHERRVFHLTVAEPHAEPPPRGSPGNGSSHSG |
| | | APGPDPTLARGHNVINVIVPES |
| 467 | STON1_0 | EFPSGSSSTSSTPLSSPIVDFYFSPGPPSNSPLSTPTKDFPGFP |
| | | GIPKAGTHVLYPIPESSS |
| 468 | STON1_1 | ISGGESSLLPTRPTCLSHALLPSDHSCTHPTPKVGLPDEVNP |
| | | QQAESLGFQSDDLPQFQYFR |
| 469 | MYBPC1 | MPEPTKKEENEVPAPAPPPEEPSKEKEAGTTPAKDWTLVE |
| | | TPPGEEQAKQNANSQLSILFIE |
| 470 | SIMC1_0 | DVPGLPQSILHPQDVAYLQDMPRSPGDVPQSPSDVSPSPD |
| | | APQSPGGMPHLPGDVLHSPGDM |
| 471 | SIMC1_1 | PQSILHPQDVAYLQDMPRSPGDVPQSPSDVSPSPDAPQSPG |
| | | GMPHLPGDVLHSPGDMPHSSG |
| 472 | SIMC1_2 | GDRPDFTQNDVQNRDMPMDISALSSPSCSPSPQSETPLEKV |
| | | PWLSVMETPARKEISLSEPAK |
| 473 | CHPF2 | FFPVHFQEFNPALSPQRSPPGPPGAGPDPPSPPGADPSRGAP |
| | | IGGRFDRQASAEGCFYNADY |
| 474 | SPATA22 | GCLPVPLFNQKKRNRQPLTSNPLKDDSGISTPSDNYDFPPL |
| | | PTDWAWEAVNPELAPVMKTVD |
| 475 | TOGARAM1 | QNPSPGAYILPSYPVSSPRTSPKHTSPLIISPKKSQDNSVNFS |
| | | NSWPLKSFEGLSKPSPQKK |
| 476 | ZCWPW1 | QNKEECGKGPKRIFAPPAQKSYSLLPCSPNSPKEETPGISSP |
| | | ETEARISLPKASLKKKEEKA |
| 477 | LTBR | TGGSMTITGNIYIYNGPVLGGPPGPGDLPATPEPPYPIPEEG |
| | | DPGPPGLSTPHQEDGKAWHL |
| 478 | TSPOAP1 | PPPCCCSIPQPCRGSGPKDLDLPPGSPGRCTPKSSEPAPATL |
| | | TGVPRRTAKKAESLSNSSHS |
| 479 | NLRP1 | TSGRRWREISASLLYQALPSSPDHESPSQESPNAPTSTAVL |
| | | GSWGSPPQPSLAPREQEAPGT |
| 480 | PLXND1_0 | VYLAAVNRLYQLSGANLSLEAEAAVGPVPDSPLCHAPQL |
| | | PQASCEHPRRLTDNYNKILQLDP |
| 481 | PLXND1_1 | LSAQWPCFWCSQQHSCVSNQSRCEASPNPTSPQDCPRTLL |
| | | SPLAPVPTGGSQNILVPLANTA |
| 482 | PLXND1_2 | SQQHSCVSNQSRCEASPNPTSPQDCPRTLLSPLAPVPTGGS |
| | | QNILVPLANTAFFQGAALECS |
| 483 | FLI1 | LSVVSDDQSLFDSAYGAAAHLPKADMTASGSPDYGQPHK |
| | | INPLPPQQEWINQPVRVNVKREY |
| 484 | COL7A1_0 | GPPGRGLTGPTGAVGLPGPPGPSGLVGPQGSPGLPGQVGE |
| | | TGKPGAPGRDGASGKDGDRGSP |
| 485 | COL7A1_1 | GEPGDPGEDGQKGAPGPKGFKGDPGVGVPGSPGPPGPPG |
| | | VKGDLGLPGLPGAPGVVGFPGQT |
| 486 | USP30 | LLGHKPSQHNPKLNKNPGPTLELQDGPGAPTPVLNQPGAP |
| | | KTQIFMNGACSPSLLPTLSAPM |
| 487 | NPAP1 | GLTSPSVQPLSGSIIPPGFAELTSPYTALGTPVNAEPVEGHN |
| | | ASAFPNGTAKTSGFRIATGM |
| 488 | RBMS3 | AASPVSTYQVQSTSWMPHPPYVMQPTGAVITPTMDHPMS |
| | | MQPANMMGPLTQQMNHLSLGTTG |
| 489 | ANKLE1_0 | VPRSQGTEAELNARLQALTLTPPNAAGFQSSPSSMPLLDRS |
| | | PAHSPPRTPTPGASDCHCLWE |
| 490 | ANKLE1_1 | LNARLQALTLTPPNAAGFQSSPSSMPLLDRSPAHSPPRTPT |
| | | PGASDCHCLWEHQTSIDSDMA |
| 491 | MEF2B | SGGRSLGEEGPPTRGASPPTPPVSIKSERLSPAPGGPGDFPK |
| | | TFPYPLLLARSLAEPLRPGP |
| 492 | VGLL2 | LAYYSKMQEAQECNASPSSSGSGSSSFSSQTPASIKEEEGS |
| | | PEKERPPEAEYINSRCVLFTY |
| 493 | ESRRB | RGSPKDERMSSHDGKCPFQSAAFTSRDQSNSPGIPNPRPSS |
| | | PTPLNERGRQISPSTRTPGGQ |
| 494 | GALNT6 | RDSMPKLQIRAPEAQQTLFSINQSCLPGFYTPAELKPFWER |
| | | PPQDPNAPGADGKAFQKSKWT |
| 495 | RBM38 | TYGLTPHYIYPPAIVQPSVVIPAAPVPSLSSPYIEYTPASPAY |
| | | AQYPPATYDQYPYAASPAT |
| 496 | COL18A1_0 | PGGRVKEGGLKGQKGEPGVPGPPGRAGPPGSPCLPGPPGL |
| | | PCPVSPLGPAGPALQTVPGPQG |
| 497 | COL18A1_1 | CPVSPLGPAGPALQTVPGPQGPPGPPGRDGTPGRDGEPGD |
| | | PGEDGKPGDTGPQGFPGTPGDV |
| 498 | COL18A1_2 | KGEPGDASLGFGMRGMPGPPGPPGPPGPPGTPVYDSNVFA |
| | | ESSRPGPPGLPGNQGPPGPKGA |
| 499 | ZMAT4 | DSHYQGKIHAKRLKLLLGEKTPLKTTATPLSPLKPPRMDT |
| | | APVVASPYQRRDSDRYCGLCAA |
| 500 | KRTAP16-1_0 | EPSCCSAVCTLPSSCQPVVCEPSCCQPVCPTPTCSVTSSCQ |
| | | AVCCDPSPCEPSCSESSICQP |
| 501 | KRTAP16-1_1 | EPPSVPSTCQEPSCCVSSICQPICSEPSPCSPAVCVSSPCQPT |
| | | CYVVKRCPSVCPEPVSCPS |
| 502 | KRTAP16-1_2 | QPTCYVVKRCPSVCPEPVSCPSTSCRPLSCSPGSSASAICRP |
| | | TCPRTFYIPSSSKRPCSATI |
| 503 | AJM1 | APGPRREDPLGRGRSYENLLGREVREPRGVSPEGRRPPVV |
| | | VNLSTSPRRYAALSLSETSLTE |
| 504 | C11orf91 | GLGPSSERPWPSPWPSGLASIPYEPLRFFYSPPPGPEVVASP |
| | | LVPCPSTPRLASASHPEELC |
| 505 | ADPGK | LLEPELPGSALRSLWSSLCLGPAPAPPGPVSPEGRLAAAW |
| | | DALIVRPVRRWRRVAVGVNACV |
| 506 | TGM1 | GDIGGNETVTLRQSFVPVRPGPRQLIASLDSPQLSQVHGVI |
| | | QVDVAPAPGDGGFFSDAGGDS |
| 507 | CACNA1C | LVHHQALAVAGLSPLLQRSHSPASFPRPFATPPATPGSRG |
| | | WPPQPVPTLRLEGVESSEKLNS |
| 508 | F12_0 | AAPPTPVSPRLHVPLMPAQPAPPKPQPTTRTPPQSQTPGAL |
| | | PAKREQPPSLTRNGPLSCGQR |
| 509 | F12_1 | VSPRLHVPLMPAQPAPPKPQPTTRTPPQSQTPGALPAKREQ |
| | | PPSLTRNGPLSCGQRLRKSLS |
| 510 | DOT1L | KNQTALDALHAQTVSQTAASSPQDAYRSPHSPFYQLPPSV |
| | | QRHSPNPLLVAPTPPALQKLLE |
| 511 | TCF7L1_0 | FAEVRRPQDSAFFKGPPYPGYPFLMIPDLSSPYLSNGPLSP |
| | | GGARTYLQMKWPLLDVPSSAT |
| 512 | TCF7L1_1 | HFSPGSPPTHLSPEIDPKTGIPRPPHPSELSPYYPLSPGAVGQ |
| | | IPHPLGWLVPQQGQPMYSL |
| 513 | CBARP_0 | PFLASPPPALGRYFSVDGGARGGPVGPCPPSPPPRRPRERS |
| | | PGPVDTRSPASSGKAPPRGGL |
| 514 | CBARP_1 | GRYFSVDGGARGGPVGPCPPSPPPRRPRERSPGPVDTRSPA |
| | | SSGKAPPRGGLTGATSPAWTR |
| 515 | SHF_0 | FEDPYSGGSSGSAALATPVAPGPTPPPRHGSPPHRLIRVETP |
| | | GPPAPPADERISGPPASSDR |
| 516 | SHF_1 | GSAALATPVAPGPTPPPRHGSPPHRLIRVETPGPPAPPADE |
| | | RISGPPASSDRLAILEDYADP |
| 517 | PTOV1 | RSGAGGPLGGRGRPPRPLVVRAVRSRSWPASPRGPQPPRI |
| | | RARSAPPMEGARVFGALGPIGP |
| 518 | HOXB13 | PAVNYAPLDLPGSAEPPKQCHPCPGVPQGTSPAPVPYGYF |
| | | GGGYYSCRVSRSSLKPCAQAAT |
| 519 | ELAVL4 | FRLDNLLNMAYGVKRLMSGPVPPSACPPRFSPITIDGMTSL |
| | | VGMNIPGHTGTGWCIFVYNLS |
| 520 | PNPLA1 | PAQPLASSTPLSLSGMPPVSFPAVHKPPSSTPGSSLPTPPPG |
| | | LSPLSPQQQVQPSGSPARSL |
| 521 | CHRD | VLCACEAPQWGRRTRGPGRVSCKNIKPECPTPACGQPRQL |
| | | PGHCCQTCPQERSSSERQPSGL |
| 522 | ALOX12 | AAPLVMLKMEPNGKLQPMVIQIQPPNPSSPTPTLFLPSDPP |
| | | LAWLLAKSWVRNSDFQLHEIQ |
| 523 | COL8A2 | GEPGLPGPPGEGRAGEPGTAGPTGPPGVPGSPGITGPPGPP |
| | | GPPGPPGAPGAFDETGIAGLH |
| 524 | NLGN4X | HNLNEIFQYVSTTTKVPPPDMTSFPYGTRRSPAKIWPTTKR |
| | | PAITPANNPKHSKDPHKTGPE |
| 525 | SMAD1 | RNLGQNEPHMPLNATFPDSFQQPNSHPFPHSPNSSYPNSPG |
| | | SSSSTYPHSPTSSDPGSPFQM |
| 526 | NID2_0 | QGNFLPLQCHGSTGFCWCVDPDGHEVPGTQTPPGSTPPHC |
| | | GPSPEPTQRPPTICERWRENLL |
| 527 | NID2_1 | PLQCHGSTGFCWCVDPDGHEVPGTQTPPGSTPPHCGPSPE |
| | | PTQRPPTICERWRENLLEHYGG |
| 528 | RNF38 | SISQDENYHHLPYAQQQAIEEPRAFHPPNVSPRLLHPAAHP |
| | | PQQNAVMVDIHDQLHQGTVPV |
| 529 | NOCT | HSPRRLCSALLQRDAPGLRRLPAPGLRRPLSPPAAVPRPAS |
| | | PRLLAAASAASGAARSCSRTV |
| 530 | ZNF746 | RPFTCTVCGKSFIRKDHLRKHQRNHAAGAKTPARGQPLPT |
| | | PPAPPDPFKSPASKGPLASTDL |
| 531 | SSH2_0 | KFPDLTVEDLETDALKADMNVHLLPMEELTSPLKDPPMSP |
| | | DPESPSPQPSCQTEISDFSTDR |
| 532 | SSH2_1 | KADMNVHLLPMEELTSPLKDPPMSPDPESPSPQPSCQTEIS |
| | | DFSTDRIDFFSALEKFVELSQ |
| 533 | ARHGAP39 | TFAPEADGTIFFPERRPSPFLKRAELPGSSSPLLAQPRKPSG |
| | | DSQPSSPRYGYEPPLYEEPP |
| 534 | WIPF1_0 | NRMPPPRPDVGSKPDSIPPPVPSTPRPIQSSPHNRGSPPVPG |
| | | GPRQPSPGPTPPPFPGNRGT |
| 535 | WIPF1_1 | PPPVPSTPRPIQSSPHNRGSPPVPGGPRQPSPGPTPPPFPGNR |
| | | GTALGGGSIRQSPLSSSSP |
| 536 | OBSCN_0 | GGSSSSSSSSDNELAPFARAKSLPPSPVTHSPLLHPRGFLRP |
| | | SASLPEEAEASERSTEAPAP |
| 537 | OBSCN_1 | NLSDLYDIKYLPFEFMIFRKVPKSAQPEPPSPMAEEELAEFP |
| | | EPTWPWPGELGPHAGLEITE |
| 538 | VWCE_0 | TATFPGEPGASPRLSPGPSTPPGAPTLPLASPGAPQPPPVTP |
| | | ERSFSASGAQIVSRWPPLPG |
| 539 | VWCE_1 | GTLLTEASALSMMDPSPSKTPITLLGPRVLSPTTSRLSTALA |
| | | ATTHPGPQQPPVGASRGEES |
| 540 | PFKFB2_0 | YGCKVETIKLNVEAVNTHRDKPTNNFPKNQTPVRMRRNS |
| | | FTPLSSSNTIRRPRNYSVGSRPL |
| 541 | PFKFB2_1 | NVEAVNTHRDKPTNNFPKNQTPVRMRRNSFTPLSSSNTIR |
| | | RPRNYSVGSRPLKPLSPLRAQD |
| 542 | NCOA6 | MILSRAQLMPQGQMMVNPPSQNLGPSPQRMTPPKQMLSQ |
| | | QGPQMMAPHNQMMGPQGQVLLQQ |
| 543 | CCDC120 | DNEEPHGCFSLAERPSPPKAWDQLRAVSGGSPERRTPWKP |
| | | PPSDLYGDLKSRRNSVASPTSP |
| 544 | ATXN7L2 | REVQGRAKDFDVLVAELKANSRKGESPKEKSPGRKEQVL |
| | | ERPSQELPSSVQVVAAVAAPSST |
| 545 | STIL | FARPQMNTRFPSSRMVPFHFPPSKCALWNPTPTGDFIYLHL |
| | | SYYRNPKLVVTEKTIRLAYRH |
| 546 | EIF4G3_0 | KQEVLPLTLELEILENPPEEMKLECIPAPITPSTVPSFPPTPPT |
| | | PPASPPHTPVIVPAAATT |
| 547 | EIF4G3_1 | LEILENPPEEMKLECIPAPITPSTVPSFPPTPPTPPASPPHTPV |
| | | IVPAAATTVSSPSAAITV |
| 548 | PRKCQ | RDTEQIFREGPVEIGLPCSIKNEARPPCLPTPGKREPQGISW |
| | | ESPLDEVDKMCHLPEPELNK |
| 549 | SCMH1 | KFPKKRGPKPGSKRKPRTLLNPPPASPTTSTPEPDTSTVPQ |
| | | DAATIPSSAMQAPTVCIYLNK |
| 550 | CABIN1 | CLVDEDSHSSAGTLPGPGASLPSSSGPGLTSPPYTATPIDHD |
| | | YVKCKKPHQQATPDDRSQDS |
| 551 | SMPD4_0 | TSDCAYFILVDRYLSWFLPTEGSVPPPLSSSPGGTSPSPPPR |
| | | TPAIPFASYGLHHTSLLKRH |
| 552 | SMPD4_1 | YFILVDRYLSWFLPTEGSVPPPLSSSPGGTSPSPPPRTPAIPF |
| | | ASYGLHHTSLLKRHISHQT |
| 553 | THAP7 | GPLGAQADEAGCSAQPSPERQPSPLEPRPVSPSAYMLRLPP |
| | | PAGAYIQNEHSYQVGSALLWK |
| 554 | EIF4G2 | QSFLMNKNQVPKLQPQITMIPPSAQPPRTQTPPLGQTPQLG |
| | | LKTNPPLIQEKPAKTSKKPPP |
| 555 | AKAP1 | GPDTAEPATAEAAVAPPDAGLPLPGLPAEGSPPPKTYVSC |
| | | LKSLLSSPTKDSKPNISAHHIS |
| 556 | ZNF684 | GCPITKTKVILKVEQGQEPWMVEGANPHESSPESDYPLVD |
| | | EPGKHRESKDNFLKSVLLTFNK |
| 557 | RGL2 | PSVSSLDSALESSPSLHSPADPSHLSPPASSPRPSRGHRRSA |
| | | SCGSPLSGGAEEASGGTGYG |
| 558 | MAP3K21 | TGATIISATGASALPLCPSPAPHSHLPREVSPKKHSTVHIVP |
| | | QRRPASLRSRSDLPQAYPQT |
| 559 | MN1_0 | GPQRPGNLPDFHSSGASSHAVPAPCLPLDQSPNRAASFHG |
| | | LPSSSGSDSHSLEPRRVTNQGA |
| 560 | MN1_1 | RCASWNGSMHNGALDNHLSPSAYPGLPGEFTPPVPDSFPS |
| | | GPPLQHPAPDHQSLQQQQQQQQ |
| 561 | FARP2_0 | PSAQPLGPPALQPGPGLSTKSPQPSPSSRKSPLSLSPAFQVP |
| | | LGPAEQGSSPLLSPVLSDAG |
| 562 | FARP2_1 | LGPPALQPGPGLSTKSPQPSPSSRKSPLSLSPAFQVPLGPAE |
| | | QGSSPLLSPVLSDAGGAGMD |
| 563 | ZNF787 | EDQQMASHENPVDILIMDDDDVPSWPPTKLSPPQSAPPAG |
| | | PPPRPRPPAPYICNECGKSFSH |
| 564 | ENKD1 | EPGPASGTESAHFLRAHSRCGPGLPPPHVSSPQPTPPGPEA |
| | | KEPGLGVDFIRHNARAAKRAP |
| 565 | DAXX | TANSIIVLDDDDEDEAAAQPGPSHPLPNAASPGAEAPSSSE |
| | | PHGARGSSSSGGKKCYKLENE |
| 566 | HIVEP1_0 | YNIAVTSSVGLTSPSSRSQVTPQNQQMDSASPLSISPANST |
| | | QSPPMPIYNSTHVASVVNQSV |
| 567 | HIVEP1_1 | TSSVGLTSPSSRSQVTPQNQQMDSASPLSISPANSTQSPPMP |
| | | IYNSTHVASVVNQSVEQMCN |
| 568 | HIVEP1_2 | EVSDLRSKSFDCGSITPPQTTPLTELQPPSSPSRVGVTGHVP |
| | | LLERRRGPLVRQISLNIAPD |
| 569 | SETBP1 | RQRGGESDFLPVSSAKPPAAPGCAGEPLLSTPGPGKGIPVG |
| | | GERMEPEEEDELGSGRDVDSN |
| 570 | SRRM2 | ATRPSPSPERSSTGPEPPAPTPLLAERHGGSPQPLATTPLSQ |
| | | EPVNPPSEASPTRDRSPPKS |
| 571 | MAPK7 | RSLLERWTRMARPAAPALTSVPAPAPAPTPTPTPVQPTSPP |
| | | PGPVAQPTGPQPQSAGSTSGP |
| 572 | ALX3 | LQNSLWASPGSGSPGGPCLVSPEGIPSPCMSPYSHPHGSVA |
| | | GFMGVPAPSAAHPGIYSIHGF |
| 573 | ATXN1L_0 | QLPSTSLQFIGSPYSLPYAVPPNFLPSPLLSPSANLATSHLPH |
| | | FVPYASLLAEGATPPPQAP |
| 574 | ATXN1L_1 | PSPLLSPSANLATSHLPHFVPYASLLAEGATPPPQAPSPAHS |
| | | FNKAPSATSPSGQLPHHSST |
| 575 | ATXN1L_2 | PYASLLAEGATPPPQAPSPAHSFNKAPSATSPSGQLPHHSS |
| | | TQPLDLAPGRMPIYYQMSRLP |
| 576 | ZZEF1_0 | IRPVDFKQRNKADKGVSLSKDPSCQTQISDSPADASPPTGL |
| | | PDAEDSEVSSQKPIEEKAVTP |
| 577 | ZZEF1_1 | FKQRNKADKGVSLSKDPSCQTQISDSPADASPPTGLPDAE |
| | | DSEVSSQKPIEEKAVTPSPEQV |
| 578 | ZNF318 | DLKVEELTALGNLGDMPVDFCTTRVSPAHRSPTVLCQKV |
| | | CEENSVSPIGCNSSDPADFEPIP |
| 579 | PDLIM4 | DPEIQDGSPTTSRRPSGTGTGPEDGRPSLGSPYGQPPRFPVP |
| | | HNGSSEATLPAQMSTLHVSP |
| 580 | CCDC9_0 | VAVTAPRKGRSVEKENVAVESEKNLGPSRRSPGTPRPPGA |
| | | SKGGRTPPQQGGRAGMGRASRS |
| 581 | CCDC9_1 | AAPRAYSDHDDRWETKEGAASPAPETPQPTSPETSPKETP |
| | | MQPPEIPAPAHRPPEDEGEENE |
| 582 | CNNM4_0 | VEAGKENMKFETGAFSYYGTMALTSVPSDRSPAHPTPLSR |
| | | SASLSYPDRTDVSTAATLAGSS |
| 583 | CNNM4_1 | ENMKFETGAFSYYGTMALTSVPSDRSPAHPTPLSRSASLS |
| | | YPDRTDVSTAATLAGSSNQFGS |
| 584 | CSF2RB | YVSSADLVFTPNSGASSVSLVPSLGLPSDQTPSLCPGLASG |
| | | PPGAPGPVKSGFEGYVELPPI |
| 585 | SPEG_0 | YMATATNELGQATCAASLTVRPGGSTSPFSSPITSDEEYLS |
| | | PPEEFPEPGETWPRTPTMKPS |
| 586 | SPEG_1 | QATCAASLTVRPGGSTSPFSSPITSDEEYLSPPEEFPEPGET |
| | | WPRTPTMKPSPSQNRRSSDT |
| 587 | SPEG_2 | ARRLQESPSLSALSEAQPSSPARPSAPKPSTPKSAEPSATTP |
| | | SDAPQPPAPQPAQDKAPEPR |
| 588 | SPEG_3 | SALSEAQPSSPARPSAPKPSTPKSAEPSATTPSDAPQPPAPQ |
| | | PAQDKAPEPRPEPVRASKPA |
| 589 | SPEG_4 | LSGHAQGPSQGPAAPPSEPKPHAAVFARVASPPPGAPEKR |
| | | VPSAGGPPVLAEKARVPTVPPR |
| 590 | ARHGAP30 | PALQHRPSPASGPGPGPGLGPGPPDEKLEASPASSPLADSG |
| | | PDDLAPALEDSLSQEVQDSFS |
| 591 | TTBK2 | KIKLGICKAATEEENSHGQANGLLNAPSLGSPIRVRSEITQP |
| | | DRDIPLVRKLRSIHSFELEK |
| 592 | POLR2A_0 | SAASDASGFSPGYSPAWSPTPGSPGSPGPSSPYIPSPGGAMS |
| | | PSYSPTSPAYEPRSPGGYTP |
| 593 | POLR2A_1 | ASGFSPGYSPAWSPTPGSPGSPGPSSPYIPSPGGAMSPSYSP |
| | | TSPAYEPRSPGGYTPQSPSY |
| 594 | POLR2A_2 | AWSPTPGSPGSPGPSSPYIPSPGGAMSPSYSPTSPAYEPRSP |
| | | GGYTPQSPSYSPTSPSYSPT |
| 595 | KLF10 | SAGGVPPMPVICQMVPLPANNPVVTTVVPSTPPSQPPAVC |
| | | PPVVFMGTQVPKGAVMFVVPQP |
| 596 | ALDOC_0 | VTEKVLAAVYKALSDHHVYLEGTLLKPNMVTPGHACPIK |
| | | YTPEEIAMATVTALRRTVPPAVP |
| 597 | ALDOC_1 | KALSDHHVYLEGTLLKPNMVTPGHACPIKYTPEEIAMATV |
| | | TALRRTVPPAVPGVTFLSGGQS |
| 598 | NEO1 | VKPPDLWIHHERLELKPIDKSPDPNPIMTDTPIPRNSQDITP |
| | | VDNSMDSNIHQRRNSYRGHE |
| 599 | DAB2_0 | PGAMMGGQPSGFSQPVIFGTSPAVSGWNQPSPFAASTPPP |
| | | VPVVWGPSASVAPNAWSTTSPL |
| 600 | DAB2_1 | SPLGNPFQSNIFPAPAVSTQPPSMHSSLLVTPPQPPPRAGPP |
| | | KDISSDAFTALDPLGDKEIK |
| 601 | GPATCH8 | KNSVTAKLLLEKIQSRKVERKPSVSEEVQATPNKAGPKLK |
| | | DPPQGYFGPKLPPSLGNKPVLP |
| 602 | TMEM131_0 | HHAHSPLEQHPQPPLPPPVPQPQEPQPERLSPAPLAHPSHPE |
| | | RASSARHSSEDSDITSLIEA |
| 603 | TMEM131_1 | LPFTTPANTLASIGLMGTENSPAPHAPSTSSPADDLGQTYN |
| | | PWRIWSPTIGRRSSDPWSNSH |
| 604 | DIP2A | NPWSISSCDAFLNVFQSRGLRPEVICPCASSPEALTVAIRRP |
| | | PDLGGPPPRKAVLSMNGLSY |
| 605 | MINK1_0 | ERTRMNKQQNSPLAKSKPGSTGPEPPIPQASPGPPGPLSQT |
| | | PPMQRPVEPQEGPHKSLVAHR |
| 606 | MINK1_1 | SPLAKSKPGSTGPEPPIPQASPGPPGPLSQTPPMQRPVEPQE |
| | | GPHKSLVAHRVPLKPYAAPV |
| 607 | IGSF9_0 | FSEIVLSAPEGLPTTPAAPGLPPTEIPPPLSPPRGLVAVRTPR |
| | | GVLLHWDPPELVPKRLDGY |
| 608 | IGSF9_1 | GLPTTPAAPGLPPTEIPPPLSPPRGLVAVRTPRGVLLHWDP |
| | | PELVPKRLDGYVLEGRQGSQG |
| 609 | IGSF9_2 | PDSVAKLKLQGSPVPSLRQSLLWGDPAGTPSPHPDPPSSRG |
| | | PLPLEPICRGPDGRFVMGPTV |
| 610 | IGSF9_3 | RTPAQRLARSFDCSSSSPSGAPQPLCIEDISPVAPPPAAPPSP |
| | | LPGPGPLLQYLSLPFFREM |
| 611 | MDC1 | PEAIAQGGQSKTLRSSTVRAMPVPTTPEFQSPVTTDQPISPE |
| | | PITQPSCIKRQRAAGNPGSL |
| 612 | NCAPH2 | GEVLASRKDFRMNTCVPHPRGAFMLEPEGMSPMEPAGVS |
| | | PMPGTQKDTGRTEEQPMEVSVCR |
| 613 | ANKIB1 | PENCCQRSGVQMPTPPPSGYNAWDTLPSPRTPRTTRSSVT |
| | | SPDEISLSPGDLDTSLCDICMC |
| 614 | UBN2_0 | KSNPTPKPTVSPSSSSPNALVAQGSHSSTNSPVHKQPSGMN |
| | | ISRQSPTLNLLPSSRTSGLPP |
| 615 | UBN2_1 | SPNALVAQGSHSSTNSPVHKQPSGMNISRQSPTLNLLPSSR |
| | | TSGLPPTKNLQAPSKLTNSSS |
| 616 | RASAL3 | EPDPEPEQEAPELEPEPELEPPTPQIPEAPTPNVPVWDIGGF |
| | | TLLDGKLVLLGGEEEGPRRP |
| 617 | TNRC6B_0 | KKKEATQKVTEQKTKVPEVTKPSLSQPTAASPIGSSPSPPV |
| | | NGGNNAKRVAVPNGQPPSAAR |
| 618 | TNRC6B_1 | TQKVTEQKTKVPEVTKPSLSQPTAASPIGSSPSPPVNGGNN |
| | | AKRVAVPNGQPPSAARYMPRE |
| 619 | TNRC6B_2 | GDPNSYNYKNVNLWDKNSQGGPAPREPNLPTPMTSKSAS |
| | | VWSKSTPPAPDNGTSAWGEPNES |
| 620 | CDAN1 | LQEEREMLRKERSKQLQQSPTPTCPTPELGSPLPSRTGSLT |
| | | DEPADPARVSSRQRLELVALV |
| 621 | KLF13 | VARILADLNQQAPAPAPAERREGAAARKARTPCRLPPPAP |
| | | EPTSPGAEGAAAAPPSPAWSEP |
| 622 | STK11IP | ELMSSFRERFGRNWLQYRSHLEPSGNPLPATPTTSAPSAPP |
| | | ASSQGPDTAPRPSPPQEEARG |
| 623 | SLC12A7_0 | VEAHADGGGDETAERTEAPGTPEGPEPERPSPGDGNPREN |
| | | SPFLNNVEVEQESFFEGKNMAL |
| 624 | SLC12A7_1 | ETAERTEAPGTPEGPEPERPSPGDGNPRENSPFLNNVEVEQ |
| | | ESFFEGKNMALFEEEMDSNPM |
| 625 | DENND5A | GSLERILVGELLTSQPEVDERPCRTPPLQQSPSVIRRLVTISP |
| | | NNKPKLNTGQIQESIGEAV |
| 626 | HIP1 | LQYFKRLIQIPQLPENPPNFLRASALSEHISPVVVIPAEASSP |
| | | DSEPVLEKDDLMDMDASQQ |
| 627 | RBM15B | YDRPLKVEPVYLRGGGGSSRRSSSSSAAASTPPPGPPAPAD |
| | | PLGYLPLHGGYQYKQRSLSPV |
| 628 | DENND4B_0 | LSGRGPKAGGRQDEAGTPRRGLGARLQQLLTPSRHSPASR |
| | | IPPPELPPDLPPPARRSPMDSL |
| 629 | DENND4B_1 | PKAGGRQDEAGTPRRGLGARLQQLLTPSRHSPASRIPPPEL |
| | | PPDLPPPARRSPMDSLLHPRE |
| 630 | DENND4B_2 | QQLLTPSRHSPASRIPPPELPPDLPPPARRSPMDSLLHPRER |
| | | PGSTASESSASLGSEWDLSE |
| 631 | MAP3K10_0 | FAEAEDGGSSVPPSPYSTPSYLSVPLPAEPSPGARAPWEPT |
| | | PSAPPARWGHGARRRCDLALL |
| 632 | MAP3K10_1 | VPPSPYSTPSYLSVPLPAEPSPGARAPWEPTPSAPPARWGH |
| | | GARRRCDLALLGCATLLGAVG |
| 633 | PAIP1_0 | AGPAERARHQPPQPKAPGFLQPPPLRQPRTTPPPGAQCEVP |
| | | ASPQRPSRPGALPEQTRPLRA |
| 634 | PAIP1_1 | QPKAPGFLQPPPLRQPRTTPPPGAQCEVPASPQRPSRPGAL |
| | | PEQTRPLRAPPSSQDKIPQQN |
| 635 | CASKIN2_0 | TESDTVKRRPKCREREPLQTALLAFGVASATPGPAAPLPSP |
| | | TPGESPPASSLPQPEPSSLPA |
| 636 | CASKIN2_1 | EPLQTALLAFGVASATPGPAAPLPSPTPGESPPASSLPQPEP |
| | | SSLPAQGVPTPLAPSPAMQP |
| 637 | CASKIN2_2 | PLPSPTPGESPPASSLPQPEPSSLPAQGVPTPLAPSPAMQPP |
| | | VPPCPGPGLESSAASRWNGE |
| 638 | CASKIN2_3 | TPGESPPASSLPQPEPSSLPAQGVPTPLAPSPAMQPPVPPCP |
| | | GPGLESSAASRWNGETEPPA |
| 639 | TFAP2E | RPDGLGAAAGGARLSSLPQAAYGPAPPLCHTPAATAAAE |
| | | FQPPYFPPPYPQPPLPYGQAPDA |
| 640 | CD5 | SRNDMCHSLGLTCLEPQKTTPPTTRPPPTTTPEPTAPPRLQ |
| | | LVAQSGGQHCAGVVEFYSGSL |
| 641 | DNAJB1 | DGRTIPVVFKDVIRPGMRRKVPGEGLPLPKTPEKRGDLIIE |
| | | FEVIFPERIPQTSRTVLEQVL |
| 642 | PALMD | DEEEEDEGEAEKPSYHPIAPHSQVYQPAKPTPLPRKRSEAS |
| | | PHENTNHKSPHKNSISLKEQE |
| 643 | RNF10 | ALGPTSTEGHGALSISPLSRSPGSHADFLLTPLSPTASQGSP |
| | | SFCVGSLEEDSPFPSFAQML |
| 644 | KMT2C_0 | PIQDSLSQAQTSQPPSPQVFSPGSSNSRPPSPMDPYAKMVG |
| | | TPRPPPVGHSFSRRNSAAPVE |
| 645 | KMT2C_1 | RETPSKAFHQYSNNISTLDVHCLPQLPEKASPPASPPIAFPP |
| | | AFEAAQVEAKPDELKVTVKL |
| 646 | SH2D3A | RTPSFELPDASERPPTYCELVPRVPSVQGTSPSQSCPEPEAP |
| | | WWEAEEDEEEENRCFTRPQA |
| 647 | PRPF6 | HTSVDPRQTQFGGLNTPYPGGLNTPYPGGMTPGLMTPGT |
| | | GELDMRKIGQARNTLMDMRLSQV |
| 648 | CDK13 | LQLRPPPEPSTPVSGQDDLIQHQDMRILELTPEPDRPRILPP |
| | | DQRPPEPPEPPPVTEEDLDY |
| 649 | ARHGAP17 | KPNSQGPPNPMALPSEHGLEQPSHTPPQTPTPPSTPPLGKQ |
| | | NPSLPAPQTLAGGNPETAQPH |
| 650 | HIVEP2_0 | SAQLFGSGKLASPSEVVQQVAEKQYPPHRPSPYSCQHSLS |
| | | FPQHSLPQGVMHSTKPHQSLEG |
| 651 | HIVEP2_1 | SESAELVACTQDKAPSPSETCDSEISEAPVSPEWAPPGDGA |
| | | ESGGKPSPSQQVQQQSYHTQP |
| 652 | MAP1S | PTSEAGLSLPLRGPRARRSASPHDVDLCLVSPCEFEHRKAV |
| | | PMAPAPASPGSSNDSSARSQE |
| 653 | ZBTB4_0 | SSSSSSSSSSSSSSSASSSSSSSSSSPPPASPPASSPPRVLELPG |
| | | VPAAAFSDVLNFIYSAR |
| 654 | ZBTB4_1 | SSSSSSSSSSASSSSSSSSSSPPPASPPASSPPRVLELPGVPAA |
| | | AFSDVLNFIYSARLALPG |
| 655 | ZBTB4_2 | NTLKLYRLLPMRAAKRPYKTYSQGAPEAPLSPTLNTPAPV |
| | | AMPASPPPGPPPAPEPGPPPSV |
| 656 | ZBTB4_3 | YRLLPMRAAKRPYKTYSQGAPEAPLSPTLNTPAPVAMPAS |
| | | PPPGPPPAPEPGPPPSVITFAH |
| 657 | NFATC3_0 | HLPQLQCRDESVSKEQHMIPSPIVHQPFQVTPTPPVGSSYQ |
| | | PMQTNVVYNGPTCLPINAASS |
| 658 | NFATC3_1 | PVADQITGQPSSQLQPITYGPSHSGSATTASPAASHPLASSP |
| | | LSGPPSPQLQPMPYQSPSSG |
| 659 | NFATC3_2 | SSQLQPITYGPSHSGSATTASPAASHPLASSPLSGPPSPQLQ |
| | | PMPYQSPSSGTASSPSPATR |
| 660 | ZBTB32 | WLRENPGGSEESLRKLPGPLPPAGSLQTSVTPRPSWAEAP |
| | | WLVGGQPALWSILLMPPRYGIP |
| 661 | DPH2 | VVLLSEPACAHALEALATLLRPRYLDLLVSSPAFPQPVGSL |
| | | SPEPMPLERFGRRFPLAPGRR |
| 662 | DMRTC2_0 | KGTTQPQVPSGKENIAPQPQTPHGAVLLAPTPPGKNSCGP |
| | | LLLSHPPEASPLSWTPVPPGPW |
| 663 | DMRTC2_1 | QTPHGAVLLAPTPPGKNSCGPLLLSHPPEASPLSWTPVPPG |
| | | PWVPGHWLPPGFSMPPPVVCR |
| 664 | DMRTC2_2 | AVLLAPTPPGKNSCGPLLLSHPPEASPLSWTPVPPGPWVPG |
| | | HWLPPGFSMPPPVVCRLLYQE |
| 665 | RBM25 | APSVSSASGNATPNTPGDESPCGIIIPHENSPDQQQPEEHRP |
| | | KIGLSLKLGASNSPGQPNSV |
| 666 | AATK | SGGDHPQAEPKLATEAEGTTGPRLPLPSVPSPSQEGAPLPS |
| | | EEASAPDAPDALPDSPTPATG |
| 667 | GATA5 | QGALLPREQFAAPLGRPVGTSYSATYPAYVSPDVAQSWT |
| | | AGPFDGSVLHGLPGRRPTFVSDF |
| 668 | CC2D1A | ASIRKGNAIDEADIPPPVAIGKGPASTPTYSPAPTQPAPRIAS |
| | | APEPRVTLEGPSATAPASS |
| 669 | NACAD | HGPRSALGGAREVPDAPPAACPEVSQARLLSPAREERGLS |
| | | GKSTPEPTLPSAVATEASLDSC |
| 670 | CUX2 | VSLNSPSAASSPGLMMSVSPVPSSSAPISPSPPGAPPAKVPS |
| | | ASPTADMAGALHPSAKVNPN |
| 671 | BSN_0 | LGASLLTQASTLMSVQPEADTQGQPAPSKGTPKIVENDAS |
| | | KEAGPKPLGSGPGPGPAPGAKT |
| 672 | BSN_1 | EPSKTPSSVQEKKTRVPTKAEPMPKPPPETTPTPATPKVKS |
| | | GVRRAEPATPVVKAVPEAPKG |
| 673 | BSN_2 | PSSVQEKKTRVPTKAEPMPKPPPETTPTPATPKVKSGVRR |
| | | AEPATPVVKAVPEAPKGGEAED |
| 674 | BSN_3 | SGGRVIPDVRVTQHFAKETQDPLKLHSSPASPSSASKEIGM |
| | | PFSQGPGTPATTAVAPCPAGL |
| 675 | BSN_4 | GPRATAEFSTQTPSPAPASDMPRSPGAPTPSPMVAQGTQTP |
| | | HRPSTPRLVWQESSQEAPFMV |
| 676 | BSN_5 | QTRMVHASASTSPLCSPTETQPTTHGYSQTTPPSVSQLPPE |
| | | PPGPPGFPRVPSAGADGPLAL |
| 677 | BSN_6 | GRGESLACQTEPDGQAQGVAGPQLVGPTAISPYLPGIQIVT |
| | | PGPLGRFEKKKPDPLEIGYQA |
| 678 | PPRC1_0 | GPLDLYPKLADTIQTNPIPTHLSLVDSAQASPMPVDSVEAD |
| | | PTAVGPVLAGPVPVDPGLVDL |
| 679 | PPRC1_1 | ISDNLPPVDAVPSGPAPVDLALVDPVPNDLTPVDPVLVKS |
| | | RPTDPRRGAVSSALGGSAPQLL |
| 680 | PPRC1_2 | PSLPETPTGLADIPCLVIPPAPAKKTALQRSPETPLEICLVPV |
| | | GPSPASPSPEPPVSKPVAS |
| 681 | PPRC1_3 | PETPTGLADIPCLVIPPAPAKKTALQRSPETPLEICLVPVGPS |
| | | PASPSPEPPVSKPVASSPT |
| 682 | PPRC1_4 | LVIPPAPAKKTALQRSPETPLEICLVPVGPSPASPSPEPPVSK |
| | | PVASSPTEQVPSQEMPLLA |
| 683 | PPRC1_5 | PPAPAKKTALQRSPETPLEICLVPVGPSPASPSPEPPVSKPV |
| | | ASSPTEQVPSQEMPLLARPS |
| 684 | PPRC1_6 | ETPLEICLVPVGPSPASPSPEPPVSKPVASSPTEQVPSQEMP |
| | | LLARPSPPVQSVSPAVPTPP |
| 685 | LMTK2 | DVMLTGDTLSTSLQSSPEVQVPPTSFETEETPRRVPPDSLPT |
| | | QGETQPTCLDVIVPEDCLHQ |
| 686 | ARNT2 | QLNQSQVAWTGSRPPFPGQQIPSQSSKTQSSPFGIGTSHTY |
| | | PADPSSYSPLSSPATSSPSGN |
| 687 | HHEX | YIEDILGRGPAAPTPAPTLPSPNSSFTSLVSPYRTPVYEPTPI |
| | | HPAFSHHSAAALAAAYGPG |
| 688 | TMEM201 | PHPSVGGSPASLFIPSPPSFLPLANQQLFRSPRRTSPSSLPGR |
| | | LSRALSLGTIPSLTRADSG |
| 689 | ALX4_0 | YGAGQQDLATPLESGAGARGSFNKFQPQPSTPQPQPPPQP |
| | | QPQQQQPQPQPPAQPHLYLQRG |
| 690 | ALX4_1 | IQNPSWLGNNGAASPVPACVVPCDPVPACMSPHAHPPGS |
| | | GASSVTDFLSVSGAGSHVGQTHM |
| 691 | MNT_0 | PLAPRQPALVGAPGLSIKEPAPLPSRPQVPTPAPLLPDSKAT |
| | | IPPNGSPKPLQPLPTPVLTI |
| 692 | MNT_1 | KEPAPLPSRPQVPTPAPLLPDSKATIPPNGSPKPLQPLPTPV |
| | | LTIAPHPGVQPQLAPQQPPP |
| 693 | MNT_2 | TTHASVIQTVNHVLQGPGGKHIAHIAPSAPSPAVQLAPATP |
| | | PIGHITVHPATLNHVAHLGSQ |
| 694 | NFATC4_0 | ASATPFGTDMDFSPPRPPYPSYPHEDPACETPYLSEGFGYG |
| | | MPPLYPQTGPPPSYRPGLRMF |
| 695 | NFATC4_1 | SDPYGGRGSSFSLGLPFSPPAPFRPPPLPASPPLEGPFPSQSD |
| | | VHPLPAEGYNKVGPGYGPG |
| 696 | TRIM33 | DNLLSRYISGSHLPPQPTSTMNPSPGPSALSPGSSGLSNSHT |
| | | PVRPPSTSSTGSRGSCGSSG |
| 697 | RBPMS | PNPSTPLPNTVPQFIAREPYELTVPALYPSSPEVWAPYPLYP |
| | | AELAPALPPPAFTYPASLHA |
| 698 | FCHSD1 | GVFPSLLVEELLGPPGPPELSDPEQMLPSPSPPSFSPPAPTSV |
| | | LDGPPAPVLPGDKALDFPG |
| 699 | SKOR1 | SAPSAGGGPDGEQPTGPPSATSSGADGPANSPDGGSPRPR |
| | | RRLGPPPAGRPAFGDLAAEDLV |
| 700 | SMG6 | QYPYTGYNPLQYPVGPTNGVYPGPYYPGYPTPSGQYVCSP |
| | | LPTSTMSPEEVEQHMRNLQQQE |
| 701 | EHBP1L1_0 | GKEAEGSLTEASLPEAQVASGAGAGAPRASSPEKAEEDRR |
| | | LPGSQAPPALVSSSQSLLEWCQ |
| 702 | EHBP1L1_1 | AAAAEGQAPDPSPAPGPPTAADSQQPPGGSSPSEEPPPSPG |
| | | EEAGLQRFQDTSQYVCAELQA |
| 703 | TAOK2 | QPKSLKVRAGQRPPGLPLPIPGALGPPNTGTPIEQQPCSPG |
| | | QEAVLDQRMLGEEEEAVGERR |
| 704 | ARHGEF5_0 | RKGTVSSQGTEVVFASASVTPPRTPDSAPPSPAEAYPITPAS |
| | | VSARPPVAFPRRETSCAARA |
| 705 | ARHGEF5_1 | GPLPQASDPAVARQHRPLPSTPDSSHHAQATPRWRYNKPL |
| | | PPTPDLPQPHLPPISAPGSSRI |
| 706 | RBM27 | LGTPPPLLAARLVPPRNLMGSSIGYHTSVSSPTPLVPDTYE |
| | | PDGYNPEAPSITSSGRSQYRQ |
| 707 | ANKRD34A_0 | GRGMLSPRAQEEEEKRDVFEFPLPKPPDDPSPSEPLPKPPR |
| | | HPPKPLKRLNSEPWGLVAPPQ |
| 708 | ANKRD34A_1 | PGLLERRGSGTLLLDHISQTRPGFLPPLNVSPHPPIPDIRPQP |
| | | GGRAPSLPAPPYAGAPGSP |
| 709 | ANKHD1 | PHFALLAAQTMQQIRHPRLPMAQFGGTFSPSPNTWGPFPV |
| | | RPVNPGNTNSSPKHNNTSRLPN |
| 710 | EPS8L2 | PVSRQSIRNSQKHSPTSEPTPPGDALPPVSSPHTHRGYQPTP |
| | | AMAKYVKILYDFTARNANEL |
| 711 | HOXD1 | PVALQPAFPLGNGDGAFVSCLPLAAARPSPSPPAAPARPSV |
| | | PPPAAPQYAQCTLEGAYEPGA |
| 712 | PPARGC1B | QSRSCTELHKHLTSAQCCLQDRGLQPPCLQSPRLPAKEDK |
| | | EPGEDCPSPQPAPASPRDSLAL |
| 713 | HUWE1_0 | PAPRGSGTASDDEFENLRIKGPNAVQLVKTTPLKPSPLPVI |
| | | PDTIKEVIYDMLNALAAYHAP |
| 714 | HUWE1_1 | SGTASDDEFENLRIKGPNAVQLVKTTPLKPSPLPVIPDTIKE |
| | | VIYDMLNALAAYHAPEEADK |
| 715 | PTPN3 | VSQNRSPHQESLSENNPAQSYLTQKSSSSVSPSSNAPGSCS |
| | | PDGVDQQLLDDFHRVTKGGST |
| 716 | SLC24A1 | VHHCVVVKPTPAMLTTPSPSLTTALLPEELSPSPSVLPPSLP |
| | | DLHPKGEYPPDLFSVEERRQ |
| 717 | DOCK2 | IISLASMNSDCSTPSKPTSESFDLELASPKTPRVEQEEPISPG |
| | | STLPEVKLRRSKKRTKRSS |
| 718 | SHARPIN | VRGATVEGQNGSKSNSPPALGPEACPVSLPSPPEASTLKGP |
| | | PPEADLPRSPGNLTEREELAG |
| 719 | KIF13B | TAVPAEEPPGPQQLVSPGRERPDLEAPAPGSPFRVRRVRAS |
| | | ELRSFSRMLAGDPGCSPGAEG |
| 720 | UNK | GSCPRGPFCAFAHVEQPPLSDDLQPSSAVSSPTQPGPVLYM |
| | | PSAAGDSVPVSPSSPHAPDLS |
| 721 | BRME1 | VETLGVPLQEATELGDPTQADSARPEQSSQSPVQAVPGSG |
| | | DSQPDDPPDRGTGLSASQRASQ |
| 722 | BICRA_0 | NSVFGGAGAASAPTGTPSGQPLAVAPGLGSSPLVPAPNVIL |
| | | HRTPTPIQPKPAGVLPPKLYQ |
| 723 | BICRA_1 | TPSGQPLAVAPGLGSSPLVPAPNVILHRTPTPIQPKPAGVLP |
| | | PKLYQLTPKPFAPAGATLTI |
| 724 | BICRA_2 | QPAPQAPPAVSTPLPLGLQQPQAQQPPQAPTPQAAAPPQA |
| | | TTPQPSPGLASSPEKIVLGQPP |
| 725 | BICRA_3 | LGLQQPQAQQPPQAPTPQAAAPPQATTPQPSPGLASSPEKI |
| | | VLGQPPSATPTAILTQDSLQM |
| 726 | BICRA_4 | PAPQIPAAAPLKGPGPSSSPSLPHQAPLGDSPHLPSPHPTRP |
| | | PSRPPSRPQSVSRPPSEPPL |
| 727 | BICRA_5 | PAAAPLKGPGPSSSPSLPHQAPLGDSPHLPSPHPTRPPSRPP |
| | | SRPQSVSRPPSEPPLHPCPP |
| 728 | MED13_0 | YTPQTHTSFGMPPSSAPPSNSGAGILPSPSTPRFPTPRTPRTP |
| | | RTPRGAGGPASAQGSVKYE |
| 729 | MED13_1 | HTSFGMPPSSAPPSNSGAGILPSPSTPRFPTPRTPRTPRTPRG |
| | | AGGPASAQGSVKYENSDLY |
| 730 | ACACB | ADVNLPAAQLQIAMGVPLHRLKDIRLLYGESPWGVTPISF |
| | | ETPSNPPLARGHVIAARITSEN |
| 731 | ERF_0 | AFRGPPLARLPHDPGVFRVYPRPRGGPEPLSPFPVSPLAGP |
| | | GSLLPPQLSPALPMTPTHLAY |
| 732 | ERF_1 | PLARLPHDPGVFRVYPRPRGGPEPLSPFPVSPLAGPGSLLPP |
| | | QLSPALPMTPTHLAYTPSPT |
| 733 | ERF_2 | YPRPRGGPEPLSPFPVSPLAGPGSLLPPQLSPALPMTPTHLA |
| | | YTPSPTLSPMYPSGGGGPSG |
| 734 | HIPK1 | QPLQIQSGVLTQGSCTPLMVATLHPQVATITPQYAVPFTLS |
| | | CAAGRPALVEQTAAVLQAWPG |
| 735 | PRR12 | GSSAPPPKAPAPPPKPETPEKTTSEKPPEQTPETAMPEPPAP |
| | | EKPSLLRPVEKEKEKEKVTR |
| 736 | INPP5D_0 | SFPKPAPRKDQESPKMPRKEPPPCPEPGILSPSIVLTKAQEA |
| | | DRGEGPGKQVPAPRLRSFTC |
| 737 | INPP5D_1 | QGKPKTPVSSQAPVPAKRPIKPSRSEINQQTPPTPTPRPPLP |
| | | VKSPAVLHLQHSKGRDYRDN |
| 738 | INPP5D_2 | TPVSSQAPVPAKRPIKPSRSEINQQTPPTPTPRPPLPVKSPA |
| | | VLHLQHSKGRDYRDNTELPH |
| 739 | SRRT | NFLTDAKRPALPEIKPAQPPGPAQILPPGLTPGLPYPHQTPQ |
| | | GLMPYGQPRPPILGYGAGAV |
| 740 | HERC1 | TLLGVVKEGSTSAKVQWDEAEITISFPTFWSPSDTPLYNLE |
| | | PCEPLPFDVARFRGLTASVLL |
| 741 | ARAP3_0 | PQAQPPKPVPKPRTVFGGLSGPATTQRPGLSPALGGPGVS |
| | | RSPEPSPRPPPLPTSSSEQSSA |
| 742 | ARAP3_1 | LGAALEMFASENSPEPLSLIQPQDIVCLGVSPPPTDPGDRFP |
| | | FSFELILAGGRIQHFGTDGA |
| 743 | PERM1 | PGPASSGDQMQRLLQGPAPRPPGEPPGSPKSPGHSTGSQRP |
| | | PDSPGAPPRSPSRKKRRAVGA |
| 744 | LNPK | PSAGAAVTARPGQEIRQRTAAQRNLSPTPASPNQGPPPQV |
| | | PVSPGPPKDSSAPGGPPERTVT |
| 745 | SYDE1 | GPAAGPGGTRSPRAGYLSDGDSPERPAGPPSPTSFRPYEVG |
| | | PAARAPPAALWGRLSLHLYGL |
| 746 | CD248 | PSQSPTNQTSPISPTHPHSKAPQIPREDGPSPKLALWLPSPA |
| | | PTAAPTALGEAGLAEHSQRD |
| 747 | OFD1 | RSLESEMYLEGLGRSHIASPSPCPDRMPLPSPTESRHSLSIPP |
| | | VSSPPEQKVGLYRRQTELQ |
| 748 | CDC27_0 | PLGTGTSILSKQVQNKPKTGRSLLGGPAALSPLTPSFGILPL |
| | | ETPSPGDGSYLQNYTNTPPV |
| 749 | CDC27_1 | TKSVFSQSGNSREVTPILAQTQSSGPQTSTTPQVLSPTITSPP |
| | | NALPRRSSRLFTSDSSTTK |
| 750 | CDC27_2 | SQSGNSREVTPILAQTQSSGPQTSTTPQVLSPTITSPPNALP |
| | | RRSSRLFTSDSSTTKENSKK |
| 751 | CDC27_3 | SREVTPILAQTQSSGPQTSTTPQVLSPTITSPPNALPRRSSRL |
| | | FTSDSSTTKENSKKLKMKF |
| 752 | PODXL | STAPSSQETVQPTSPATALRTPTLPETMSSSPTAASTTHRYP |
| | | KTPSPTVAHESNWAKCEDLE |
| 753 | PODXL2 | PTADYVFPDLTEKAGSIEDTSQAQELPNLPSPLPKMNLVEP |
| | | PWHMPPREEEEEEEEEEEREK |
| 754 | TELO2_0 | RQRMDILDVLTLAAQELSRPGCLGRTPQPGSPSPNTPCLPE |
| | | AAVSQPGSAVASDWRVVVEER |
| 755 | TELO2_1 | ILDVLTLAAQELSRPGCLGRTPQPGSPSPNTPCLPEAAVSQ |
| | | PGSAVASDWRVVVEERIRSKT |
| 756 | CNTROB | TKVPLAMASSLFRVPEPPSSHSQGSGPSSGSPERGGDGLTF |
| | | PRQLMEVSQLLRLYQARGWGA |
| 757 | CIZ1_0 | QFAMPPATYDTAGLTMPTATLGNLRGYGMASPGLAAPSL |
| | | TPPQLATPNLQQFFPQATRQSLL |
| 758 | CIZ1_1 | MPTATLGNLRGYGMASPGLAAPSLTPPQLATPNLQQFFPQ |
| | | ATRQSLLGPPPVGVPMNPSQFN |
| 759 | NUP98 | GSHELENHQIADSMEFGFLPNPVAVKPLTESPFKVHLEKLS |
| | | LRQRKPDEDMKLYQTPLELKL |
| 760 | MEF2D | NQSSLQFSNPSGSLVTPSLVTSSLTDPRLLSPQQPALQRNS |
| | | VSPGLPQRPASAGAMLGGDLN |
| 761 | HMX3 | FALSQVGDLAFPRFEIPAQRFALPAHYLERSPAWWYPYTL |
| | | TPAGGHLPRPEASEKALLRDSS |
| 762 | FOXB1 | GDYSAYGVPLKPLCHAAGQTLPAIPVPIKPTPAAVPALPAL |
| | | PAPIPTLLSNSPPSLSPTSSQ |
| 763 | USP43 | SPPRPQPGHCDGDGEGGFACAPGPVPAAPGSPGEERPPGP |
| | | QPQLQLPAGDGARPPGAQGLKN |
| 764 | MLXIPL_0 | PMAPPTALLQEEPLFSPRFPFPTVPPAPGVSPLPAPAAFPPT |
| | | PQSVPSPAPTPFPIELLPLG |
| 765 | MLXIPL_1 | VSSTLLRSPGSPQETVPEFPCTFLPPTPAPTPPRPPPGPATLA |
| | | PSRPLLVPKAERLSPPAPS |
| 766 | SLX4 | PGAHRPKGPAKTKGPRHQRKHHESITPPSRSPTKEAPPGLN |
| | | DDAQIPASQESVATSVDGSDS |
| 767 | SCAP | MLPPSHPDPAFSIFPPDAPKLPENQTSPGESPERGGPAEVV |
| | | HDSPVPEVTWGPEDEELWRKL |
| 768 | RPAP1 | LQDHRDVVMLDNLPDLPPALVPSPPKRARPSPGHCLPEDE |
| | | DPEERLRRHDQHITAVLTKIIE |
| 769 | IQSEC2_0 | SYSHPHHPQSPLSPHSPIPPHPSYPPLPPPSPHTPHSPLPPTSP |
| | | HGPLHASGPPGTANPPSA |
| 770 | IQSEC2_1 | HPHHPQSPLSPHSPIPPHPSYPPLPPPSPHTPHSPLPPTSPHGP |
| | | LHASGPPGTANPPSANPK |
| 771 | IQSEC2_2 | SPHSPIPPHPSYPPLPPPSPHTPHSPLPPTSPHGPLHASGPPGT |
| | | ANPPSANPKAKPSRISTV |
| 772 | PDLIM7_0 | GTEFMQDPDEEHLKKSSQVPRTEAPAPASSTPQEPWPGPT |
| | | APSPTSRPPWAVDPAFAERYAP |
| 773 | PDLIM7_1 | LKKSSQVPRTEAPAPASSTPQEPWPGPTAPSPTSRPPWAVD |
| | | PAFAERYAPDKTSTVLTRHSQ |
| 774 | ZC3H12D | AALRGSFSRLAFSDDLGPLGPPLPVPACSLTPRLGGPDWVS |
| | | AGGRVPGPLSLPSPESQFSPG |
| 775 | IRX5 | TAPSPGYNSHLQYGADPAAAAAAAFSSYVGSPYDHTPGM |
| | | AGSLGYHPYAAPLGSYPYGDPAY |
| 776 | TACC2_0 | HRDASSIGSVGLGGFCTASESSASLDPCLVSPEVTEPRKDP |
| | | QGARGPEGSLLPSPPPSQERE |
| 777 | TACC2_1 | RGTKPNQVVCVAAGGQPEGGLPVSPEPSLLTPTEEAHPAS |
| | | SLASFPAAQIPIAVEEPGSSSR |
| 778 | TACC2_2 | DNQQENPPPTKKIGKKPVAKMPLRRPKMKKTPEKLDNTP |
| | | ASPPRSPAEPNDIPIAKGTYTFD |
| 779 | ANKLE2 | SPSDRQSWPSPAVKGRFKSQLPDLSGPHSYSPGRNSVAGS |
| | | NPAKPGLGSPGRYSPVHGSQLR |
| 780 | RAP1GAP2 | AGEGEAMEEGDSGGSQPSTTSPFKQEVFVYSPSPSSESPSL |
| | | GAAATPIIMSRSPTDAKSRNS |
| 781 | SLC26A9 | ENAPPTDPNNNQTPANGTSVSYITFSPDSSSPAQSEPPASAE |
| | | APGEPSDMLASVPPFVTFHT |
| 782 | MAP1A_0 | SQYGTPVFSAPGHALHPGEPALGEAEERCLSPDDSTVKMA |
| | | SPPPSGPPSATHTPFHQSPVEE |
| 783 | MAP1A_1 | SSPQKGLEVERWLAESPVGLPPEEEDKLTRSPFEIISPPASPP |
| | | EMVGQRVPSAPGQESPIPD |
| 784 | MAP1A_2 | HMKNEPTTPSWLADIPPWVPKDRPLPPAPLSPAPGPPTPAP |
| | | ESHTPAPFSWGTAEYDSVVAA |
| 785 | MAP1A_3 | TPSWLADIPPWVPKDRPLPPAPLSPAPGPPTPAPESHTPAPF |
| | | SWGTAEYDSVVAAVQEGAAE |
| 786 | MAP1A_4 | SDTPTFSYAALAGPTVPPRPEPGPSMEPSLTPPAVPPRAPIL |
| | | SKGPSPPLNGNILSCSPDRR |
| 787 | MAP1A_5 | RFSPSLEAAEQESGELDPGMEPAAHSLWDLTPLSPAPPASL |
| | | DLALAPAPSLPGDMGDGILPC |
| 788 | DOCK4 | TQTASPARHTTSVSPSPAGRSPLKGSVQSFTPSPVEYHSPG |
| | | LISNSPVLSGSYSSGISSLSR |
| 789 | CEP350 | LDSTAHTAKQDTVELQNQKSSAPVHAPRSHSPVKRKPDKI |
| | | TANEDPPVISKRRHYDTDEVRQ |
| 790 | MAML2 | PFNIDLGQQSQRSTPRPSLPMEKIVIKSEYSPGLTQGPSGSP |
| | | QLRPPSAGPAFSMANSALST |
| 791 | ATAD5 | FFNSYYIGKSPKKISSPKKVVTSPRKVPPPSPKSSGPKRALP |
| | | PKTLANYFKVSPKPKNNEEI |
| 792 | SMAP2 | PVPEKKLEPVVFEKVKMPQKKEDPQLPRKSSPKSTAPVMD |
| | | LLGLDAPVACSIANSKTSNTLE |
| 793 | PTPN23_0 | GPTQLIQPRAPGPHAMPVAPGPALYPAPAYTPELGLVPRSS |
| | | PQHGVVSSPYVGVGPAPPVAG |
| 794 | PTPN23_1 | GPQAAPLTIRGPSSAGQSTPSPHLVPSPAPSPGPGPVPPRPP |
| | | AAEPPPCLRRGAAAADLLSS |
| 795 | PTPN23_2 | QDLVLGGDVPISSIQATIAKLSIRPPGGLESPVASLPGPAEPP |
| | | GLPPASLPESTPIPSSSPP |
| 796 | PTPN23_3 | LPGPAEPPGLPPASLPESTPIPSSSPPPLSSPLPEAPQPKEEPP |
| | | VPEAPSSGPPSSSLELLA |
| 797 | CASC3_0 | HGDSPAPLPPQGMLVQPGMNLPHPGLHPHQTPAPLPNPGL |
| | | YPPPVSMSPGQPPPQQLLAPTY |
| 798 | CASC3_1 | GMNLPHPGLHPHQTPAPLPNPGLYPPPVSMSPGQPPPQQL |
| | | LAPTYFSAPGVMNFGNPSYPYA |
| 799 | GOLGA3 | KVQCAEVNRASTEGESPDGPGQGGLCQNGPTPPFPDPPSS |
| | | LDPTTSPVGPDASPGVAGFHDN |
| 800 | MISP_0 | RRLCDLERERWAVIQGQAVRKSSTVATLQGTPDHGDPRT |
| | | PGPPRSTPLEENVVDREQIDFLA |
| 801 | MISP_1 | GQAVRKSSTVATLQGTPDHGDPRTPGPPRSTPLEENVVDR |
| | | EQIDFLAARQQFLSLEQANKGA |
| 802 | PROSER2_0 | PPDPPAPETLLAPPPLPSTPDPPRRELRAPSPPVEHPRLLRSV |
| | | PTPLVMAQKISERMAGNEA |
| 803 | PROSER2_1 | MAQKISERMAGNEALSPTSPFREGRPGEWRTPAARGPRSG |
| | | DPGPGPSHPAQPKAPRFPSNII |
| 804 | DTL | EDLSKDSLGPTKSSKIEGAGTSISEPPSPISPYASESCGTLPL |
| | | PLRPCGEGSEMVGKENSSP |
| 805 | TOX4_0 | YLKALAAYKDNQECQATVETVELDPAPPSQTPSPPPMATV |
| | | DPASPAPASIEPPALSPSIVVN |
| 806 | TOX4_1 | APPSQTPSPPPMATVDPASPAPASIEPPALSPSIVVNSTLSSY |
| | | VANQASSGAGGQPNITKLI |
| 807 | TOX4_2 | IKSVPLPTLKMQTTLVPPTVESSPERPMNNSPEAHTVEAPS |
| | | PETICEMITDVVPEVESPSQM |
| 808 | CASKIN1_0 | GPAPATAKVKPTPQLLPPTERPMSPRSLPQSPTHRGFAYVL |
| | | PQPVEGEVGPAAPGPAPPPVP |
| 809 | CASKIN1_1 | PPPEGEARKPAKPPVSPKPVLTQPVPKLQGSPTPTSKKVPL |
| | | PGPGSPEVKRAHGTPPPVSPK |
| 810 | CASKIN1_2 | PEGEARKPAKPPVSPKPVLTQPVPKLQGSPTPTSKKVPLPG |
| | | PGSPEVKRAHGTPPPVSPKPP |
| 811 | CASKIN1_3 | VAGLPSGSAGPSPAPSPARQPPAALAKPPGTPPSLGASPAK |
| | | PPSPGAPALHVPAKPPRAAAA |
| 812 | SRGAP3 | RLRSDGAAIPRRRSGGDTHSPPRGLGPSIDTPPRAAACPSSP |
| | | HKIPLTRGRIESPEKRRMAT |
| 813 | CSTF2T | PPLMQTPIQGGIPAPGPIPAAVPGAGPGSLTPGGAMQPQLG |
| | | MPGVGPVPLERGQVQMSDPRA |
| 814 | ADNP2 | TQPVGPINRPVGPGVLPVSPSVTPGVLQAVSPGVLSVSRAV |
| | | PSGVLPAGQMTPAGQMTPAGV |
| 815 | PRR36_0 | PKPKGLQALRPPQVTPPRKDAAPALGPLSSSPLATPSPSGT |
| | | KARPVPPPDNAATPLPATLPP |
| 816 | PRR36_1 | HSSSLTCQLATPLPLAPPSPSAPPSLQTLPSPPATPPSQVPPT |
| | | QLIMSFPEAGVSSLATAAF |
| 817 | PRR36_2 | ASVSPSVSSPLQSMPPTQANPALPSLPTLLSPLATPPLSAMS |
| | | PLQGPVSPATSLGNSAFPLA |
| 818 | PRR36_3 | LQGPVSPATSLGNSAFPLAALPQPGLSALTTPPPQASPSPSP |
| | | PSLQATPHTLATLPLQDSPL |
| 819 | PRR36_4 | ETPPCPAPCPLQAPPSPLTTPPPETPSSIATPPPQAPPALASPP |
| | | LQGLPSPPLSPLATPPPQ |
| 820 | PRR36_5 | ETPSSIATPPPQAPPALASPPLQGLPSPPLSPLATPPPQAPPA |
| | | LALPPLQAPPSPPASPPLS |
| 821 | PRR36_6 | SIATPPPQAPPALASPPLQGLPSPPLSPLATPPPQAPPALALP |
| | | PLQAPPSPPASPPLSPLAT |
| 822 | PRR36_7 | PSPQAPNALAVHLLQAPFSPPPSPPVQAPFSPPASPPVSPSA |
| | | TPPSQAPPSLAAPPLQVPPS |
| 823 | PRR36_8 | LAVHLLQAPFSPPPSPPVQAPFSPPASPPVSPSATPPSQAPPS |
| | | LAAPPLQVPPSPPASPPMS |
| 824 | PRR36_9 | PSATPPSQAPPSLAAPPLQVPPSPPASPPMSPSATPPPQAPPP |
| | | LAAPPLQVPPSPPASPPMS |
| 825 | PRR36_10 | PSATPPPQAPPPLAAPPLQVPPSPPASPPMSPSATPPPRVPPL |
| | | LAAPPLQVPPSPPASLPMS |
| 826 | PRR36_11 | PSATPPPRVPPLLAAPPLQVPPSPPASLPMSPLAKPPPQAPP |
| | | ALATPPLQALPSPPASFPGQ |
| 827 | PRR36_12 | PPLQVPPSPPASLPMSPLAKPPPQAPPALATPPLQALPSPPA |
| | | SFPGQAPFSPSASLPMSPLA |
| 828 | PRR36_13 | LATPPLQALPSPPASFPGQAPFSPSASLPMSPLATPPPQAPP |
| | | VLAAPLLQVPPSPPASPTLQ |
| 829 | SOX18_0 | APGHGAAADTRGLAAGPAALAAPAAPASPPSPQRSPPRSP |
| | | EPGRYGLSPAGRGERQAADESR |
| 830 | SOX18_1 | GGCYGAPLAEALRTAPPAAPLAGLYYGTLGTPGPYPGPLS |
| | | PPPEAPPLESAEPLGPAADLWA |
| 831 | SOX18_2 | EALRTAPPAAPLAGLYYGTLGTPGPYPGPLSPPPEAPPLES |
| | | AEPLGPAADLWADVDLTEFDQ |
| 832 | DDI2 | QKENADPRPPVQFPNLPRIDFSSIAVPGTSSPRQRQPPGTQQ |
| | | SHSSPGEITSSPQGLDNPAL |
| 833 | TRIM47 | CPEGAALPAALSCLSCLASFCPAHLGPHERSPALRGHRLVP |
| | | PLRRLEESLCPRHLRPLERYC |
| 834 | SF3B2 | AHKVPPPWLIAMQRYGPPPSYPNLKIPGLNSPIPESCSFGY |
| | | HAGGWGKPPVDETGKPLYGDV |
| 835 | TBC1D25 | LLSDWDLSTAFATASKPYLQLRVDIRPSEDSPLLEDWDIIS |
| | | PKDVIGSDVLLAEKRSSLTTA |
| 836 | HCFC1 | SADGKPTTIITTTQASGAGTKPTILGISSVSPSTTKPGTTTIIK |
| | | TIPMSAIITQAGATGVTS |
| 837 | NEUROD6_0 | TPPGHGTLDNSKSMKPYNYCSAYESFYESTSPECASPQFE |
| | | GPLSPPPINYNGIFSLKQEETL |
| 838 | NEUROD6_1 | GTLDNSKSMKPYNYCSAYESFYESTSPECASPQFEGPLSPP |
| | | PINYNGIFSLKQEETLDYGKN |
| 839 | PPP1R3D | SRKLGPRSLSCLSDLDGGVALEPRACRPPGSPGRAPPPTPA |
| | | PSGCDPRLRPIILRRARSLPS |
| 840 | CACNA1I_0 | NFLCEMEEIPFNPVRSWLKHDSSQAPPSPFSPDASSPLLPM |
| | | PAEFFHPAVSASQKGPEKGTG |
| 841 | CACNA1I_1 | MEEIPFNPVRSWLKHDSSQAPPSPFSPDASSPLLPMPAEFF |
| | | HPAVSASQKGPEKGTGTGTLP |
| 842 | ZFPM1 | LLLGAPLAGPGVEARTPADRGPSPAPAPAASPQPGSRGPR |
| | | DGLGPEPQEPPPGPPPSPAAAP |
| 843 | SETD1A | PVPERVAGSPVTPLPEQEASPARPAGPTEESPPSAPLRPPEP |
| | | PAGPPAPAPRPDERPSSPIP |
| 844 | KEL | SLNFNRTLRLLMSQYGHFPFFRAYLGPHPASPHTPVIQIDQ |
| | | PEFDVPLKQDQEQKIYAQIFR |
| 845 | CCDC102A_0 | ESPQLSKGSLLTILGSPSPERMGPADSLPPTPPSGTPSPGPPP |
| | | ALPLPPAPALLADGDWESR |
| 846 | CCDC102A_1 | GSLLTILGSPSPERMGPADSLPPTPPSGTPSPGPPPALPLPPA |
| | | PALLADGDWESREELRLRE |
| 847 | NIBAN2 | TEIRGLLAQGLRPESPPPAGPLLNGAPAGESPQPKAAPEAS |
| | | SPPASPLQHLLPGKAVDLGPP |
| 848 | TANC2_0 | EEEYLEQDVENVSIGLQTEARPSQGLPVIQSPPSSPPHRDSA |
| | | YISSSPLGSHQVFDFRSSSS |
| 849 | TANC2_1 | SSSQLGSPDVSHLIRRPISVNPNEIKPHPPTPRPLLHSQSVGL |
| | | RFSPSSNSISSTSNLTPTF |
| 850 | EPOP_0 | ASAPPRPAPGLEPQRGPAASPPQEPSSRPPSPPAGLSTEPAG |
| | | PGTAPRPFLPGQPAEVDGNP |
| 851 | EPOP_1 | PGTAPRPFLPGQPAEVDGNPPPAAPEAPAASPSTASPAPAA |
| | | PGDLRQEHFDRLIRRSKLWCY |
| 852 | EPOP_2 | RPFLPGQPAEVDGNPPPAAPEAPAASPSTASPAPAAPGDLR |
| | | QEHFDRLIRRSKLWCYAKGFA |
| 853 | ICE1_0 | GSTEFVDHDHFFDEDLQAAIDFFKLPPPLLSPVPSPPPMSSP |
| | | HPGSLPSSFAPETYFGEYTD |
| 854 | ICE1_1 | FFDEDLQAAIDFFKLPPPLLSPVPSPPPMSSPHPGSLPSSFAP |
| | | ETYFGEYTDSSDNDSVQLR |
| 855 | ICE1_2 | PLISSSSPSSPASPVGQVSPFRETPVPPAMSPWPEDPRRASP |
| | | PDPSPSPSAASASERVVPSP |
| 856 | ICE1_3 | PASPVGQVSPFRETPVPPAMSPWPEDPRRASPPDPSPSPSA |
| | | ASASERVVPSPLQFCAATPKH |
| 857 | ICE1_4 | GQVSPFRETPVPPAMSPWPEDPRRASPPDPSPSPSAASASE |
| | | RVVPSPLQFCAATPKHALPVP |
| 858 | ZBED4 | TSCLIRHMWRAHRAIVLQENGGTGIPPLYSTPPTLLPSLLPP |
| | | EGELSSVSSSPVKPVRESPS |
| 859 | CAMSAP1 | ELKDAKTVLHQKSSRPPVPISNATKRSFLGSPAAGTLAELQ |
| | | PPVQLPAEGCHRHYLHPEEPE |
| 860 | TBC1D17 | ELPHNVQEILGLAPPAEPHSPSPTASPLPLSPTRAPPTPPPST |
| | | DTAPQPDSSLEILPEEEDE |
| 861 | SLC12A9 | LGFYDDAPPQDHFLTDPAFSEPADSTREGSSPALSTLFPPPR |
| | | APGSPRALNPQDYVATVADA |
| 862 | DLG3 | ISHNSSLGYLGAVESKVSYPAPPQVPPTRYSPIPRHMLAEE |
| | | DFTREPRKIILHKGSTGLGFN |
| 863 | SCARF1 | GAQSGPEGREAEESTGPEEAEAPESFPAAASPGDSATGHR |
| | | RPPLGGRTVAEHVEAIEGSVQE |
| 864 | PRRX2_0 | MLASRSASLLKSYSQEAAIEQPVAPRPTALSPDYLSWTASS |
| | | PYSTVPPYSPGSSGPATPGVN |
| 865 | PRRX2_1 | KSYSQEAAIEQPVAPRPTALSPDYLSWTASSPYSTVPPYSP |
| | | GSSGPATPGVNMANSIASLRL |
| 866 | DOK2 | EEAISAQKNAAPATPQPQPATIPASLPRPDSPYSRPHDSLPP |
| | | PSPTTPVPAPRPRGQEGEYA |
| 867 | ATF7 | GCGMVVGTASTMVTARPEQSQILIQHPDAPSPAQPQVSPA |
| | | QPTPSTGGRRRRTVDEDPDERR |
| 868 | UBQLN4 | QTEAPGLVPSLGSFGISRTPAPSAGSNAGSTPEAPTSSPATP |
| | | ATSSPTGASSAQQQLMQQMI |
| 869 | TANK | ACLPPGDHNALYVNSFPLLDPSDAPFPSLDSPGKAIRGPQQ |
| | | PIWKPFPNQDSDSVVLSGTDS |
| 870 | PDE12 | FGDPASSLFRWYKEAKPGAAEPEVGVPSSLSPSSPSSSWTE |
| | | TDVEERVYTPSNADIGLRLKL |
| 871 | RABL6 | ASPLAANGQSPSPGSQSPVVPAGAVSTGSSSPGTPQPAPQL |
| | | PLNAAPPSSVPPVPPSEALPP |
| 872 | WNK1 | AVAPSKLLTSTTSTCLPPTNLPLGTVALPVTPVVTPGQVST |
| | | PVSTTTSGVKPGTAPSKPPLT |
| 873 | MORC2 | RSQADLKKLPLEVTTRPSTEEPVRRPQRPRSPPLPAVIRNA |
| | | PSRPPSLPTPRPASQPRKAPV |
| 874 | MED12_0 | GVSSHSSHVISAQSTSTLPTTPAPQPPTSSTPSTPFSDLLMCP |
| | | QHRPLVFGLSCILQTILLC |
| 875 | MED12_1 | IDPSSSVLFEDMEKPDFSLFSPTMPCEGKGSPSPEKPDVEKE |
| | | VKPPPKEKIEGTLGVLYDQP |
| 876 | CDT1 | EKALSQLALRSAAPSSPGSPRPALPATPPATPPAASPSALK |
| | | GVSQDLLERIRAKEAQKQLAQ |
| 877 | CIPC | LQSWTVQPSFEVISAQPQLLFLHPPVPSPVSPCHTGEKKSD |
| | | SRNYLPILNSYTKIAPHPGKR |
| 878 | RBPMS2 | ARDPYDLMGAALIPASPEAWAPYPLYTTELTPAISHAAFT |
| | | YPTATAAAAALHAQVRWYPSSD |
| 879 | EPN3 | ASGSSWGPSADPWSPIPSGTVLSRSQPWDLTPMLSSSEPW |
| | | GRTPVLPAGPPTTDPWALNSPH |
| 880 | FRAT1 | LRCALGDRGRVRGRAAPYCVAELATGPSALSPLPPQADL |
| | | DGPPGAGKQGIPQPLSGPCRRGW |
| 881 | RERE_0 | PQDNESDSDSSAQQQMLQAQPPALQAPTGVTPAPSSAPPG |
| | | TPQLPTPGPTPSATAVPPQGSP |
| 882 | RERE_1 | SAQQQMLQAQPPALQAPTGVTPAPSSAPPGTPQLPTPGPTP |
| | | SATAVPPQGSPTASQAPNQPQ |
| 883 | RERE_2 | MLQAQPPALQAPTGVTPAPSSAPPGTPQLPTPGPTPSATAV |
| | | PPQGSPTASQAPNQPQAPTAP |
| 884 | RERE_3 | TPAPSSAPPGTPQLPTPGPTPSATAVPPQGSPTASQAPNQPQ |
| | | APTAPVPHTHIQQAPALHPQ |
| 885 | RERE_4 | QSALQSQQPPREQPLPPAPLAMPHIKPPPTTPIPQLPAPQAH |
| | | KHPPHLSGPSPFSMNANLPP |
| 886 | RERE_5 | RFPYPPGTLPNPLLGQPPHEHEMLRHPVFGTPYPRDLPGAI |
| | | PPPMSAAHQLQAMHAQSAELQ |
| 887 | ETV5 | YGEKCLYNYCAYDRKPPSGFKPLTPPTTPLSPTHQNPLFPP |
| | | PQATLPTSGHAPAAGPVQGVG |
| 888 | SYNJ2 | ASEEALSAVAPRDLEASSEPEPTPGAAKPETPQAPPLLPRR |
| | | PPPRVPAIKKPTLRRTGKPLS |
| 889 | NBR1_0 | TAQDLLSFELLDINIVQELERVPHNTPVDVTPCMSPLPHDS |
| | | PLIEKPGLGQIEEENEGAGFK |
| 890 | NBR1_1 | LDINIVQELERVPHNTPVDVTPCMSPLPHDSPLIEKPGLGQI |
| | | EEENEGAGFKALPDSMVSVK |
| 891 | NBR1_2 | QTLETVPLIPEVVELPPSLPRSSPCVHHHGSPGVDLPVTIPE |
| | | VSSVPDQIRGEPRGSSGLVN |
| 892 | NCKAP5L | TSHFTACGSLTRTLDSGIGTFPPPDHGSSGTPSKNLPKTKPP |
| | | RLDPPPGVPPARPPPLTKVP |
| 893 | KIF1C_0 | PFKSNPQHRESWPGMGSGEAPTPLQPPEEVTPHPATPARR |
| | | PPSPRRSHHPRRNSLDGGGRSR |
| 894 | KIF1C_1 | PQHRESWPGMGSGEAPTPLQPPEEVTPHPATPARRPPSPRR |
| | | SHHPRRNSLDGGGRSRGAGSA |
| 895 | PHLDB1 | AMSVGSSYENTSPAFSPLSSPASSGSCASHSPSGQEPGPSVP |
| | | PLVPARSSSYHLALQPPQSR |
| 896 | EIF3F | APASSSDPAAAAAATAAPGQTPASAQAPAQTPAPALPGPA |
| | | LPGPFPGGRVVRLHPVILASIV |
| 897 | UBE2O | EEKMEAVPDVERKEDKPEGQSPVKAEWPSETPVLCQQCG |
| | | GKPGVTFTSAKGEVFSVLEFAPS |
| 898 | YLPM1_0 | KQQQYKHQMLHHQRDGPPGLVPMELESPPESPPVPPGSY |
| | | MPPSQSYMPPPQPPPSYYPPTSS |
| 899 | YLPM1_1 | PSQSYMPPPQPPPSYYPPTSSQPYLPPAQPSPSQSPPSQSYL |
| | | APTPSYSSSSSSSQSYLSHS |
| 900 | YLPM1_2 | GHKKGPVVAKDTPEPVKEEVTVPATSQVPESPSSEEPPLPP |
| | | PNEEVPPPLPPEEPQSEDPEE |
| 901 | YLPM1_3 | SAGPPPVLPPPSLSSTAPPPVMPLPPLSSATPPPGIPPPGVPQ |
| | | GIPPQLTAAPVPPASSSQS |
| 902 | CDC42BPB | EPSVTVPLRSMSDPDQDFDKEPDSDSTKHSTPSNSSNPSGP |
| | | PSPNSPHRSQLPLEGLEQPAC |
| 903 | MAP3K6 | AALGVLGPEVEKEAVSPRSEELSNEGDSQQSPGQQSPLPV |
| | | EPEQGPAPLMVQLSLLRAETDR |
| 904 | PKN3_0 | RGQDFLRASQMNLGMAAWGRLVMNLLPPCSSPSTISPPK |
| | | GCPRTPTTLREASDPATPSNFLP |
| 905 | PKN3_1 | LRASQMNLGMAAWGRLVMNLLPPCSSPSTISPPKGCPRTP |
| | | TTLREASDPATPSNFLPKKTPL |
| 906 | PKN3_2 | LPKKTPLGEEMTPPPKPPRLYLPQEPTSEETPRTKRPHMEP |
| | | RTRRGPSPPASPTRKPPRLQD |
| 907 | NUAK2_0 | GKSNLKLPKGILKKKVSASAEGVQEDPPELSPIPASPGQAA |
| | | PLLPKKGILKKPRQRESGYYS |
| 908 | NUAK2_1 | KLPKGILKKKVSASAEGVQEDPPELSPIPASPGQAAPLLPK |
| | | KGILKKPRQRESGYYSSPEPS |
| 909 | CEP104 | YEQLELHSLLDAELMRRPFDLPLQPLARSGSPCHQKPMPS |
| | | LPQLEERGTENQFAEPFLQEKP |
| 910 | MAST3_0 | SSEDEGVGPGPAGPKRPVFILGEPDPPPAATPVMPKPSSLS |
| | | ADTAALSHARLRSNSIGARHS |
| 911 | MAST3_1 | LPGSPTHSLSPSPTTPCRSPAPDVPADTTASPPSASPSSSSPA |
| | | SPAAAGHTRPSSLHGLAAK |
| 912 | MAST3_2 | THSLSPSPTTPCRSPAPDVPADTTASPPSASPSSSSPASPAA |
| | | AGHTRPSSLHGLAAKLGPPR |
| 913 | MAST3_3 | RPSSLHGLAAKLGPPRPKTGRRKSTSSIPPSPLACPPISAPPP |
| | | RSPSPLPGHPPAPARSPRL |
| 914 | MAST3_4 | PRPKTGRRKSTSSIPPSPLACPPISAPPPRSPSPLPGHPPAPAR |
| | | SPRLRRGQSADKLGTGER |
| 915 | WNK4_0 | HRSWTAFSTSSSSPGTPLSPGNPFSPGTPISPGPIFPITSPPCH |
| | | PSPSPFSPISSQVSSNPS |
| 916 | WNK4_1 | TPLSPGNPFSPGTPISPGPIFPITSPPCHPSPSPFSPISSQVSSNP |
| | | SPHPTSSPLPFSSSTP |
| 917 | WNK4_2 | GNPFSPGTPISPGPIFPITSPPCHPSPSPFSPISSQVSSNPSPHP |
| | | TSSPLPFSSSTPEFPVP |
| 918 | WNK4_3 | SPSPFSPISSQVSSNPSPHPTSSPLPFSSSTPEFPVPLSQCPWS |
| | | SLPTTSPPTFSPTCSQVT |
| 919 | WNK4_4 | SAFSLAVMTVAQSLLSPSPGLLSQSPPAPPSPLPSLPLPPPV |
| | | APGGQESPSPHTAEVESEAS |
| 920 | CTTNBP2NL | NTANPRGDTSHSPTPGKVSSPLSPLSPGIKSPTIPRAERGNP |
| | | PPIPPKKPGLTPSPSATTPL |
| 921 | TAF3_0 | KVKDKGREDKMKAPAPPLVLPPKELALPLFSPATASRVPA |
| | | MLPSLLPVLPEKLFEEKEKVKE |
| 922 | TAF3_1 | RVGAGQDKIVISKVVPAPEAKPAPSQNRPKTPPPAPAPAPG |
| | | PMLVSPAPVPLPLLAQAAAGP |
| 923 | TAF3_2 | PAPEAKPAPSQNRPKTPPPAPAPAPGPMLVSPAPVPLPLLA |
| | | QAAAGPALLPSPGPAASGASA |
| 924 | C1orf116_0 | LIPPPEAFRDTQPEQCREASLPEGPGQQGHTPQLHTPSSSQE |
| | | REQTPSEAMSQKAKETVSTR |
| 925 | C1orf116_1 | EAFRDTQPEQCREASLPEGPGQQGHTPQLHTPSSSQEREQT |
| | | PSEAMSQKAKETVSTRYTQPQ |
| 926 | PHACTR4 | ITTKTPSDEREKSTCSMGSELLPMISPRSPSPPLPTHIPPEPPR |
| | | TPPFPAKTFQVVPEIEFP |
| 927 | PARP10 | TLEGLDLDGEDWLPRELEEEGPQEQPEEEVTPGHEEEEPV |
| | | APSTVAPRWLEEEAALQLALHR |
| 928 | SH3RF3 | GSCPIESEMQGAMGMEPLHRKAGSLDLNFTSPSRQAPLSM |
| | | AAIRPEPKLLPRERYRVVVSYP |
| 929 | MED1_0 | RKKADTEGKSPSHSSSNRPFTPPTSTGGSKSPGSAGRSQTP |
| | | PGVATPPIPKITIQIPKGTVM |
| 930 | MED1_1 | SNRPFTPPTSTGGSKSPGSAGRSQTPPGVATPPIPKITIQIPK |
| | | GTVMVGKPSSHSQYTSSGS |
| 931 | MED1_2 | GLSSGSSSTKMKPQGKPSSLMNPSLSKPNISPSHSRPPGGS |
| | | DKLASPMKPVPGTPPSSKAKS |
| 932 | MED1_3 | KPSSLMNPSLSKPNISPSHSRPPGGSDKLASPMKPVPGTPPS |
| | | SKAKSPISSGSGGSHMSGTS |
| 933 | ELL | GDQQLLKRVLVRKLCQPQSTGSLLGDPAASSPPGERGRSA |
| | | SPPQKRLQPPDFIDPLANKKPR |
| 934 | CASP9 | LEDTGQDMLASFLRTNRQAAKLSKPTLENLTPVVLRPEIR |
| | | KPEVLRPETPRPVDIGSGGFGD |
| 935 | PPFIA3 | SRVSSSGLDSLGRYRSSCSLPPSLTTSTLASPSPPSSGHSTPR |
| | | LAPPSPAREGTDKANHVPK |
| 936 | GAK_0 | DLLSCLLGPPEAASQGPPEDLLSEDPLLLASPAPPLSVQSTP |
| | | RGGPPAAADPFGPLLPSSGN |
| 937 | GAK_1 | EAASQGPPEDLLSEDPLLLASPAPPLSVQSTPRGGPPAAAD |
| | | PFGPLLPSSGNNSQPCSNPDL |
| 938 | GAK_2 | APCGSQASWTKSQNPDPFADLGDLSSGLQGSPAGFPPGGF |
| | | IPKTATTPKGSSSWQTSRPPAQ |
| 939 | RAPH1 | QAAPPTPTPPVPPAKKQPAFPASYIPPSPPTPPVPVPPPTLPK |
| | | QQSFCAKPPPSPLSPVPSV |
| 940 | NOTO | SRVRPPRSGRSPAPRSPTGPNTPRAPGRFESPFSVEAILARP |
| | | DPCAPAASQPSGSACVHPAF |
| 941 | SNAI3 | PRASRAAIVPLKDSLNHLNLPPLLVLPTRWSPTLGPDRHG |
| | | APEKLLGAERMPRAPGGFECFH |
| 942 | CYP4F22 | IYGTHHNPTVWPDSKVYNPYRFDPDNPQQRSPLAYVPFSA |
| | | GPRNCIGQSFAMAELRVVVALT |
| 943 | BCL9_0 | EMNRMIPGSQRHMEPGNNPIFPRIPVEGPLSPSRGDFPKGIP |
| | | PQMGPGRELEFGMVPSGMKG |
| 944 | BCL9_1 | PGINPLKSPTMHQVQSPMLGSPSGNLKSPQTPSQLAGMLA |
| | | GPAAAASIKSPPVLGSAAASPV |
| 945 | BCL9_2 | AGMLAGPAAAASIKSPPVLGSAAASPVHLKSPSLPAPSPG |
| | | WTSSPKPPLQSPGIPPNHKAPL |
| 946 | UTF1 | ATPLPTARDRDADPTWTLRFSPSPPKSADASPAPGSPPAPA |
| | | PTALATCIPEDRAPVRGPGSP |
| 947 | MICALL2_0 | GGMAGVKRASEDSEEEPSGKKAPVQAAKLPSPAPARKPP |
| | | LSPAQTNPVVQRRNEGAGGPPPK |
| 948 | MICALL2_1 | KDSSKEQARNFLKQALSALEEAGAPAPGRPSPATAAVPSS |
| | | QPKTEAPQASPLAKPLQSSSPR |
| 949 | MICALL2_2 | EEEKKPHLQGKPGRPLSPANVPALPGETVTSPVRLHPDYL |
| | | SPEEIQRQLQDIERRLDALELR |
| 950 | POU6F1_0 | PQLLLNAQGQVIATLASSPLPPPVAVRKPSTPESPAKSEVQ |
| | | PIQPTPTVPQPAVVIASPAPA |
| 951 | POU6F1_1 | ASSPLPPPVAVRKPSTPESPAKSEVQPIQPTPTVPQPAVVIA |
| | | SPAPAAKPSASAPIPITCSE |
| 952 | MICAL3 | DAPSDLKAVHSPIRSQPVTLPEARTPVSPGSPQPQPPVAAS |
| | | TPPPSPLPICSQPQPSTEATV |
| 953 | ASH1L | VFSLQSKEEQEPPILQPEIEIPSFKQGLSVSPFPKKRGRPKRQ |
| | | MRSPVKMKPPVLSVAPFVA |
| 954 | LCP2 | DEDDVHQRPLPQPALLPMSSNTFPSRSTKPSPMNPLPSSHM |
| | | PGAFSESNSSFPQSASLPPYF |
| 955 | LHX5 | PLGALEPPLAGPHAADNPRFTDMISHPDTPSPEPGLPGTLH |
| | | PMPGEVFSGGPSPPFPMSGTS |
| 956 | PRICKLE3 | EYAWVPPGLKPEQVYQFFSCLPEDKVPYVNSPGEKYRIKQ |
| | | LLHQLPPHDSEAQYCTALEEEE |
| 957 | MAP3K1_0 | NSPSGRTVKSESPGVRRKRVSPVPFQSGRITPPRRAPSPDGF |
| | | SPYSPEETNRRVNKVMRARL |
| 958 | MAP3K1_1 | MVQTKGRPHSQCLNSSPLSHHSQLMFPALSTPSSSTPSVPA |
| | | GTATDVSKHRLQGFIPCRIPS |
| 959 | DYNC1LI1 | KLQSLLAKQPPTAAGRPVDASPRVPGGSPRTPNRSVSSNV |
| | | ASVSPIPAGSKKIDPNMKAGAT |
| 960 | ZFHX3 | FDNTPLQALNLPTAYPALQGIPPVLLPGLNSPSLPGFTPSNT |
| | | ALTSPKPNLMGLPSTTVPSP |
| 961 | CCNO | LHPLNPCPLPGDSGICDLFESPSSGSDGAESPSAARGGSPLP |
| | | GPAQPVAQLDLQTFRDYGQS |
| 962 | WAC_0 | SHSCTTPSTSSASGLNPTSAPPTSASAVPVSPVPQSPIPPLLQ |
| | | DPNLLRQLLPALQATLQLN |
| 963 | WAC_1 | SPRISTPQTNTVPIKPLISTPPVSSQPKVSTPVVKQGPVSQSA |
| | | TQQPVTADKQQGHEPVSPR |
| 964 | SCML2 | LPTQQVRRSSRIKPPGPTAVPKRSSSVKNITPRKKGPNSGK |
| | | KEKPLPVICSTSAASLKSLTR |
| 965 | ZNF512B | CGKTYRSKAGHDYHVRSEHTAPPPEEPTDKSPEAEDPLGV |
| | | ERTPSGRVRRTSAQVAVFHLQE |
| 966 | SCYL1_0 | AVTGVSSLTSKLIRSHPTTAPTETNIPQRPTPEGVPAPAPTP |
| | | VPATPTTSGHWETQEEDKDT |
| 967 | SCYL1_1 | KLIRSHPTTAPTETNIPQRPTPEGVPAPAPTPVPATPTTSGH |
| | | WETQEEDKDTAEDSSTADRW |
| 968 | TRIOBP_0 | ISRASSTQQETSRASSTQEDTPRASSTQEDTPRASSTQWNT |
| | | PRASSPSRSTQLDNPRTSSTQ |
| 969 | TRIOBP_1 | AAYGAPLTSPEPSQPPCAVCIGHRDAPRASSPPRYLQHDPF |
| | | PFFPEPRAPESEPPHHEPPYI |
| 970 | TRIOBP_2 | RAPESEPPHHEPPYIPPAVCIGHRDAPRASSPPRHTQFDPFP |
| | | FLPDTSDAEHQCQSPQHEPL |
| 971 | TRIOBP_3 | AEHQCQSPQHEPLQLPAPVCIGYRDAPRASSPPRQAPEPSL |
| | | LFQDLPRASTESLVPSMDSLH |
| 972 | TRIOBP_4 | SLVPSMDSLHECPHIPTPVCIGHRDAPSFSSPPRQAPEPSLFF |
| | | QDPPGTSMESLAPSTDSLH |
| 973 | TRIOBP_5 | SLAPSTDSLHGSPVLIPQVCIGHRDAPRASSPPRHPPSDLAF |
| | | LAPSPSPGSSGGSRGSAPPG |
| 974 | NELFA | LNNEPALPSTSYLPSTPSVVPASSYIPSSETPPAPSSREASRP |
| | | PEEPSAPSPTLPAQFKQRA |
| 975 | BCR | ASASRPQPAPADGADPPPAEEPEARPDGEGSPGKARPGTA |
| | | RRPGAAASGERDDRGPPASVAA |
| 976 | EPS15_0 | VSNVVITKNVFEETSVKSEDEPPALPPKIGTPTRPCPLPPGK |
| | | RSINKLDSPDPFKLNDPFQP |
| 977 | EPS15_1 | LPPGKRSINKLDSPDPFKLNDPFQPFPGNDSPKEKDPEIFCD |
| | | PFTSATTTTNKEADPSNFAN |
| 978 | JCAD | HSQQQSPTEKAGASGQPPSGPPGTGNEYGVSPRLPQGLPA |
| | | HPRPVTAYDGFVQYIPFDDPRL |
| 979 | EP400 | QAAQLAGQRQSQQQYDPSTGPPVQNAASLHTPLPQLPGR |
| | | LPPAGVPTAALSSALQFAQQPQV |
| 980 | SGIP1 | ESAFDEQKTEVLLDQPEIWGSGQPINPSMESPKLTRPFPTG |
| | | TPPPLPPKNVPATPPRTGSPL |
| 981 | FBXO42 | GQCVVVFSQAPSGRAPLSPSLNSRPSPISATPPALVPETREY |
| | | RSQSPVRSMDEAPCVNGRWG |
| 982 | SP2_0 | SPLALLAATCSKIGPPAVEAAVTPPAPPQPTPRKLVPIKPAP |
| | | LPLSPGKNSFGILSSKGNIL |
| 983 | SP2_1 | PAVEAAVTPPAPPQPTPRKLVPIKPAPLPLSPGKNSFGILSS |
| | | KGNILQIQGSQLSASYPGGQ |
| 984 | COL4A1_0 | PGSPGLPGPKGEPGKIVPLPGPPGAEGLPGSPGFPGPQGDR |
| | | GFPGTPGRPGLPGEKGAVGQP |
| 985 | COL4A1_1 | IVPLPGPPGAEGLPGSPGFPGPQGDRGFPGTPGRPGLPGEK |
| | | GAVGQPGIGFPGPPGPKGVDG |
| 986 | CHAF1B_0 | VLNMRTPDTAKKTKSQTHRGSSPGPRPVEGTPASRTQDPS |
| | | SPGTTPPQARQAPAPTVIRDPP |
| 987 | CHAF1B_1 | KKTKSQTHRGSSPGPRPVEGTPASRTQDPSSPGTTPPQARQ |
| | | APAPTVIRDPPSITPAVKSPL |
| 988 | C6orf132_0 | RSPAEPKGSALGPNPEPHLTFPRSFKVPPPTPVRTSSIPVQE |
| | | AQEAPRKEEGATKKAPSRLP |
| 989 | C6orf132_1 | KNLPPQSTTLLPTTSLQPKAMLGPAIPPKATPEPAIPPKATL |
| | | WPATPPKATLGPATPLKATS |
| 990 | C6orf132_2 | LQPKAMLGPAIPPKATPEPAIPPKATLWPATPPKATLGPAT |
| | | PLKATSGPTTPLKATSGPAIA |
| 991 | PCGF2_0 | SGASECESVSDKAPSPATLPATSSSLPSPATPSHGSPSSHGP |
| | | PATHPTSPTPPSTASGATTA |
| 992 | PCGF2_1 | CESVSDKAPSPATLPATSSSLPSPATPSHGSPSSHGPPATHP |
| | | TSPTPPSTASGATTAANGGS |
| 993 | PCGF2_2 | ATSSSLPSPATPSHGSPSSHGPPATHPTSPTPPSTASGATTA |
| | | ANGGSLNCLQTPSSTSRGRK |
| 994 | SRCAP_0 | GPALLTSVTPPLAPVVPAAPGPPSLAPSGASPSASALTLGL |
| | | ATAPSLSSSQTPGHPLLLAPT |
| 995 | SRCAP_1 | GAASTLVPGVSETSASPGSPSVRSMSGPESSPPIGGPCEAAP |
| | | SSSLPTPPQQPFIARRHIEL |
| 996 | SRCAP_2 | IVADPVLEPQLIPGPQPLGPQPVHRPNPLLSPVEKRRRGRPP |
| | | KARDLPIPGTISSAGDGNSE |
| 997 | SYNPO2_0 | RMVPMNRTAKPFPGSVNQPATPFSPTRNMTSPIADFPAPPP |
| | | YSAVTPPPDAFSRGVSSPIAG |
| 998 | SYNPO2_1 | MKQALPPRPVNAASPTNVQASSVYSVPAYTSPPSFFAEAS |
| | | SPVSASPVPVGIPTSPKQESAS |
| 999 | SYNPO2_2 | NAASPTNVQASSVYSVPAYTSPPSFFAEASSPVSASPVPVG |
| | | IPTSPKQESASSSYFVAPRPK |
| 1000 | CHRNA10_0 | ARALLLGHLARGLCVRERGEPCGQSRPPELSPSPQSPEGG |
| | | AGPPAGPCHEPRCLCRQEALLH |
| 1001 | CHRNA10_1 | LGHLARGLCVRERGEPCGQSRPPELSPSPQSPEGGAGPPAG |
| | | PCHEPRCLCRQEALLHHVATI |
| 1002 | KIAA1522_0 | LPRPPTTGGSEGAGAAPCPPNPANSWVPGLSPGGSRRPPRS |
| | | PERTLSPSSGYSSQSGTPTLP |
| 1003 | KIAA1522_1 | APSDRSGPQILTPLGDRFVIPPHPKVPAPFSPPPSKPRSPNPA |
| | | APALAAPAVVPGPVSTTDA |
| 1004 | KIAA1522_2 | MADFPPPEEAFFSVASPEPAGPSGSPELVSSPAASSSSATAL |
| | | QIQPPGSPDPPPAPPAPAPA |
| 1005 | KIAA1522_3 | SPETQADLQRNLVAELRSISEQRPPQAPKKSPKAPPPVARK |
| | | PSVGVPPPASPSYPRAEPLTA |
| 1006 | KIAA1522_4 | EQRPPQAPKKSPKAPPPVARKPSVGVPPPASPSYPRAEPLT |
| | | APPTNGLPHTQDRTKRELAEN |
| 1007 | BCLAF1_0 | DEFNKSSATSGDIWPGLSAYDNSPRSPHSPSPIATPPSQSSS |
| | | CSDAPMLSTVHSAKNTPSQH |
| 1008 | BCLAF1_1 | KNTPSQHSHSIQHSPERSGSGSVGNGSSRYSPSQNSPIHHIP |
| | | SRRSPAKTIAPQNAPRDESR |
| 1009 | BCLAF1_2 | QHSHSIQHSPERSGSGSVGNGSSRYSPSQNSPIHHIPSRRSP |
| | | AKTIAPQNAPRDESRGRSSF |
| 1010 | BCLAF1_3 | ERSGSGSVGNGSSRYSPSQNSPIHHIPSRRSPAKTIAPQNAP |
| | | RDESRGRSSFYPDGGDQETA |
| 1011 | JPH1 | DYVKQRFQEGVDAKENPEEKVPEKPPTPKESPHFYRKGTT |
| | | PPRSPEASPKHSHSPASSPKPL |
| 1012 | NCOA2 | YALKMNSPSQSSPGMNPGQPTSMLSPRHRMSPGVAGSPRI |
| | | PPSQFSPAGSLHSPVGVCSSTG |
| 1013 | RBSN | AVAGNPFIQPDSPAPNPFSEEDEHPQQRLSSPLVPGNPFEEP |
| | | TCINPFEMDSDSGPEAEEPI |
| 1014 | PDLIM5 | LDSPTSGRPGVTSLTAAAAFKPVGSTGVIKSPSWQRPNQG |
| | | VPSTGRISNSATYSGSVAPANS |
| 1015 | HOXC4 | RGHGPAQAGHHHPEKSQSLCEPAPLSGASASPSPAPPACS |
| | | QPAPDHPSSAASKQPIVYPWMK |
| 1016 | PPP1R13L_0 | GSPRKAATDGADTPFGRSESAPTLHPYSPLSPKGRPSSPRT |
| | | PLYLQPDAYGSLDRATSPRPR |
| 1017 | PPP1R13L_1 | LQPQPQPQPQPQSQPQPQLPPQPQTQPQTPTPAPQHPQQT |
| | | WPPVNEGPPKPPTELEPEPEIE |
| 1018 | PPP1R13L_2 | HPQQTWPPVNEGPPKPPTELEPEPEIEGLLTPVLEAGDVDE |
| | | GPVARPLSPTRLQPALPPEAQ |
| 1019 | FAM184A | NRFVSVPNLSALESGGVGNGHPNRLDPIPNSPVHDIEFNSS |
| | | KPLPQPVPPKGPKTFLSPAQS |
| 1020 | SCRIB | YRALAAVPSAGSVQRVPSGAAGGKMAESPCSPSGQQPPSP |
| | | PSPDELPANVKQAYRAFAAVPT |
| 1021 | ARHGEF17_0 | RGAWPSVTEMRKLFGGPGSRRPSADSESPGTPSPDGAAW |
| | | EPPARESRQPPTPPPRTCFPLAG |
| 1022 | ARHGEF17_1 | IAVCSARILCIGAVPGLQPRCHREPPPSLRSPPETAPEPAGP |
| | | ELDVEAAADEEAATLAEPGP |
| 1023 | ATN1_0 | SDSSSGLSQGPARPYHPPPLFPPSPQPPDSTPRQPEASFEPHP |
| | | SVTPTGYHAPMEPPTSRMF |
| 1024 | ATN1_1 | ASGPPLSATQIKQEPAEEYETPESPVPPARSPSPPPKVVDVP |
| | | SHASQSARFNKHLDRGFNSC |
| 1025 | ARMH4 | KTEKFEADTDHRTTSFPGAESTAGSEPGSLTPDKEKPSQM |
| | | TADNTQAAATKQPLETSEYTLS |
| 1026 | TSC22D4_0 | YEGPGSPGASDPPTPQPPTGPPPRLPNGEPSPDPGGKGTPR |
| | | NGSPPPGAPSSRFRVVKLPHG |
| 1027 | TSC22D4_1 | ASDPPTPQPPTGPPPRLPNGEPSPDPGGKGTPRNGSPPPGAP |
| | | SSRFRVVKLPHGLGEPYRRG |
| 1028 | TSC22D4_2 | TPQPPTGPPPRLPNGEPSPDPGGKGTPRNGSPPPGAPSSRFR |
| | | VVKLPHGLGEPYRRGRWTCV |
| 1029 | BCAR3_0 | HGTLPRKKKGPPPIRSCDDFSHMGTLPHSKSPRQNSPVTQ |
| | | DGIQESPWQDRHGETFTFRDPH |
| 1030 | BCAR3_1 | RKKKGPPPIRSCDDFSHMGTLPHSKSPRQNSPVTQDGIQES |
| | | PWQDRHGETFTFRDPHLLDPT |
| 1031 | SMAD5_0 | LLVQFRNLSHNEPHMPQNATFPDSFHQPNNTPFPLSPNSPY |
| | | PPSPASSTYPNSPASSGPGSP |
| 1032 | SMAD5_1 | RNLSHNEPHMPQNATFPDSFHQPNNTPFPLSPNSPYPPSPA |
| | | SSTYPNSPASSGPGSPFQLPA |
| 1033 | ARGFX | KKQQQQQSAKQRNQILPSKKNVPTSPRTSPSPYAFSPVISD |
| | | FYSSLPSQPLDPSNWAWNSTF |
| 1034 | SYNPO_0 | VLRPEPTKQPPYQLRPSLFVLSPIKEPAKVSPRAASPAKPSS |
| | | LDLVPNLPKGALPPSPALPR |
| 1035 | SYNPO_1 | PTKQPPYQLRPSLFVLSPIKEPAKVSPRAASPAKPSSLDLVP |
| | | NLPKGALPPSPALPRPSRSS |
| 1036 | CHAMP1_0 | PEHQKIPCNSAEPKSIPALSMETQKLGSVLSPESPKPTPLTP |
| | | LEPQKPGSVVSPELQTPLPS |
| 1037 | CHAMP1_1 | SPEPPKSVPVCESQKLAPVPSPEPQKPAPVSPESVKATLSNP |
| | | KPQKQSHFPETLGPPSASSP |
| 1038 | PLEKHA7_0 | KNPERKTVPLFPHPPVPSLSTSESKPPPQPSPPTSPVRTPLEV |
| | | RLFPQLQTYVPYRPHPPQL |
| 1039 | PLEKHA7_1 | LEVRLFPQLQTYVPYRPHPPQLRKVTSPLQSPTKAKPKVE |
| | | DEAPPRPPLPELYSPEDQPPAV |
| 1040 | PLEKHA7_2 | KVTSPLQSPTKAKPKVEDEAPPRPPLPELYSPEDQPPAVPP |
| | | LPREATIIRHTSVRGLKRQSD |
| 1041 | SEC24C | SQPNHVSSPPQALPPGTQMTGPLGPLPPMHSPQQPGYQPQ |
| | | QNGSFGPARGPQSNYGGPYPAA |
| 1042 | ARHGEF10 | QAPSAPETGGAGASEAPAPTGGEDGAGAETTPVAEPTKLV |
| | | LPMKVNPYSVIDITPFQEDQPP |
| 1043 | EVL | SEAGRKPWERSNSVEKPVSSILSRTPSVAKSPEAKSPLQSQ |
| | | PHSRMKPAGSVNDMALDAFDL |
| 1044 | PLIN1_0 | AERRASGAPSAGPEPAPRLAQPRRSLRSAQSPGAPPGPGLE |
| | | DEVATPAAPRPGFPAVPREKP |
| 1045 | PLIN1_1 | APRLAQPRRSLRSAQSPGAPPGPGLEDEVATPAAPRPGFPA |
| | | VPREKPKRRVSDSFFRPSVME |
| 1046 | THRAP3 | WPDATYGTGSASRASAVSELSPRERSPALKSPLQSVVVRR |
| | | RSPRPSPVPKPSPPLSSTSQMG |
| 1047 | PLEKHG4 | VLSEGPGPSGVESLLCPMSSHLSLAQGESDTPGVGLVGDP |
| | | GPSRAMPSGLSPGALDSDPVGL |
| 1048 | FNBP4 | DSTLANFLAEIDAITAPQPAAPVGASAPPPTPPRPEPKEAAT |
| | | STLSSSTSNGTDSTQTSGWQ |
| 1049 | RREB1_0 | EEAGSSEQPSPCPAPGPSLPVTLGPSGILESPMAPAPAATPE |
| | | PPAQPLQGPVQLAVPIYSSA |
| 1050 | RREB1_1 | ASATKDCSHREEKVTAGWPSEPGQGDLNPESPAALGQDL |
| | | LEPRSKRPAHPILATADGASQLV |
| 1051 | IRX2_0 | LKQPSLGPGCGPPGLPAAAAPASTGAPPGGSPYPASPLLGR |
| | | PLYYTSPFYGNYTNYGNLNAA |
| 1052 | IRX2_1 | LGPGCGPPGLPAAAAPASTGAPPGGSPYPASPLLGRPLYYT |
| | | SPFYGNYTNYGNLNAALQGQG |
| 1053 | PDHX | DALKLVQLKQTGKITESRPTPAPTATPTAPSPLQATAGPSY |
| | | PRPVIPPVSTPGQPNAVGTFT |
| 1054 | SALL2 | PFSAGGVGRSHKPTPAPSPALPGSTDQLIASPHLAFPSTTGL |
| | | LAAQCLGAARGLEATASPGL |
| 1055 | AUTS2 | PLSTQPPQGPPEAQLQPAPQPQVQRPPRPQSPTQLLHQNLP |
| | | PVQAHPSAQSLSQPLSAYNSS |
| 1056 | FOSL1_0 | MSGSQELQWMVQPHFLGPSSYPRPLTYPQYSPPQPRPGVI |
| | | RALGPPPGVRRRPCEQISPEEE |
| 1057 | FOSL1_1 | RPVPCISLSPGPVLEPEALHTPTLMTTPSLTPFTPSLVFTYPS |
| | | TPEPCASAHRKSSSSSGDP |
| 1058 | BSX | KPLREVAPDHFASSLASRVPLLDYGYPLMPTPTLLAPHAH |
| | | HPLHKGDHHHPYFLTTSGMPVP |
| 1059 | PRRC2A_0 | VSSGPCSQRSSPDGGLKGAAEGPPKRPGGSSPLNAVPCEG |
| | | PPGSEPPRRPPPAPHDGDRKEL |
| 1060 | PRRC2A_1 | PLSLLPVGPALQPPSLAVRPPPAPATRVLPSPARPFPASLGR |
| | | AELHPVELKPFQDYQKLSSN |
| 1061 | DBNDD1 | AEVFADSDDENLNTESPAGLHPLPRAGYLRSPSWTRTRAE |
| | | QSHEKQPLGDPERQATVLDTFL |
| 1062 | TENT2 | YSLVLMVLHYLQTLPEPILPSLQKIYPESFSPAIQLHLVHQA |
| | | PCNVPPYLSKNESNLGDLLL |
| 1063 | PACS2_0 | VVKVGIVEPSSATSGDSDDAAPSGSGTLSSTPPSASPAAKE |
| | | ASPTPPSSPSVSGGLSSPSQG |
| 1064 | PACS2_1 | IVEPSSATSGDSDDAAPSGSGTLSSTPPSASPAAKEASPTPP |
| | | SSPSVSGGLSSPSQGVGAEL |
| 1065 | GRAMD1A | RASSDADHGAEEDKEEQVDSQPDASSSQTVTPVAEPPSTE |
| | | PTQPDGPTTLGPLDLLPSEELL |
| 1066 | CHD4_0 | KVQEFEHVNGRWSMPELAEVEENKKMSQPGSPSPKTPTPS |
| | | TPGDTQPNTPAPVPPAEDGIKI |
| 1067 | CHD4_1 | EHVNGRWSMPELAEVEENKKMSQPGSPSPKTPTPSTPGDT |
| | | QPNTPAPVPPAEDGIKIEENSL |
| 1068 | CHD4_2 | VNGRWSMPELAEVEENKKMSQPGSPSPKTPTPSTPGDTQP |
| | | NTPAPVPPAEDGIKIEENSLKE |
| 1069 | CHD4_3 | RWSMPELAEVEENKKMSQPGSPSPKTPTPSTPGDTQPNTP |
| | | APVPPAEDGIKIEENSLKEEES |
| 1070 | FAM168A | ASSAAFRYTAGTPYKVPPTQSNTAPPPYSPSPNPYQTAMY |
| | | PIRSAYPQQNLYAQGAYYTQPV |
| 1071 | HOXD12 | FYFSNLRPNGGQLAALPPISYPRGALPWAATPASCAPAQP |
| | | AGATAFGGFSQPYLAGSGPLGL |
| 1072 | CEP85 | PHSNSSGVLPLGLQPAPGLSKPLPSQVWQPSPDTWHPREQ |
| | | SCELSTCRQQLELIRLQMEQMQ |
| 1073 | EIF4G1 | DDRSQGAIIADRPGLPGPEHSPSESQPSSPSPTPSPSPVLEPG |
| | | SEPNLAVLSIPGDTMTTIQ |
| 1074 | FCHO1_0 | SPENVEDSGLDSPSHAAPGPSPDSWVPRPGTPQSPPSCRAP |
| | | PPEARGIRAPPLPDSPQPLAS |
| 1075 | FCHO1_1 | QSPPSCRAPPPEARGIRAPPLPDSPQPLASSPGPWGLEALA |
| | | GGDLMPAPADPTAREGLAAPP |
| 1076 | USP25 | LSYGSGPKRFPLVDVLQYALEFASSKPVCTSPVDDIDASSP |
| | | PSGSIPSQTLPSTTEQQGALS |
| 1077 | RXRB | EQQTPEPEPGEAGRDGMGDSGRDSRSPDSSSPNPLPQGVP |
| | | PPSPPGPPLPPSTAPSLGGSGA |
| 1078 | SNW1 | MQKDPMEPPRFKINKKIPRGPPSPPAPVMHSPSRKMTVKE |
| | | QQEWKIPPCISNWKNAKGYTIP |
| 1079 | APC_0 | KKQNLKNNSKVFNDKLPNNEDRVRGSFAFDSPHHYTPIEG |
| | | TPYCFSRNDSLSSLDFDDDDVD |
| 1080 | APC_1 | SRGRTMIHIPGVRNSSSSTSPVSKKGPPLKTPASKSPSEGQT |
| | | ATTSPRGAKPSVKSELSPVA |
| 1081 | APC_2 | MIHIPGVRNSSSSTSPVSKKGPPLKTPASKSPSEGQTATTSP |
| | | RGAKPSVKSELSPVARQTSQ |
| 1082 | APC_3 | SSSTSPVSKKGPPLKTPASKSPSEGQTATTSPRGAKPSVKSE |
| | | LSPVARQTSQIGGSSKAPSR |
| 1083 | RAPGEF6 | SQSQDDSIVGTRHCRHSLAIMPIPGTLSSSSPDLLQPTTSML |
| | | DFSNPSDIPDQVIRVFKVDQ |
| 1084 | SMTN | EPPLEPAEAQCLTAEVPGSPEPPPSPPKTTSPEPQESPTLPST |
| | | EGQVVNKLLSGPKETPAAQ |
| 1085 | PKN1 | TGTLEVRVVGCRDLPETIPWNPTPSMGGPGTPDSRPPFLSR |
| | | PARGLYSRSGSLSGRSSLKAE |
| 1086 | ASXL2_0 | FQVSPQPFLNRGDRIQVRKVPPLKIPVSRISPMPFHPSQVSP |
| | | RARFPVSITSPNRTGARTLA |
| 1087 | ASXL2_1 | RGDRIQVRKVPPLKIPVSRISPMPFHPSQVSPRARFPVSITSP |
| | | NRTGARTLADIKAKAQLVK |
| 1088 | ASXL2_2 | FSSTVLPLPADSPTHQPLLLPPLQTPKLYGSPTQIGPSYRGM |
| | | INVSTSSDMDHNSAVPGSQV |
| 1089 | AOC1 | NENIENEDLVAWVTVGFLHIPHSEDIPNTATPGNSVGFLLR |
| | | PFNFFPEDPSLASRDTVIVWP |
| 1090 | MAP3K7 | ISGNGQPRRRSIQDLTVTGTEPGQVSSRSSSPSVRMITTSGP |
| | | TSEKPTRSHPWTPDDSTDTN |
| 1091 | TEPSIN | PLPGSQVFLQPLSSTPVSSRSPAPSSGMPSSPVPTPPPDASPI |
| | | PAPGDPSEAEARLAESRRW |
| 1092 | KIDINS220 | HSGKRGIPHSLSGLQDPIIARMSICSEDKKSPSECSLIASSPE |
| | | ENWPACQKAYNLNRTPSTV |
| 1093 | CAPRIN1_0 | FTSGEKEQVDEWTVETVEVVNSLQQQPQAASPSVPEPHSL |
| | | TPVAQADPLVRRQRVQDLMAQM |
| 1094 | CAPRIN1_1 | EWTVETVEVVNSLQQQPQAASPSVPEPHSLTPVAQADPLV |
| | | RRQRVQDLMAQMQGPYNFIQDS |
| 1095 | TEAD4 | PGQAGTSHDVKPFSQQTYAVQPPLPLPGFESPAGPAPSPSA |
| | | PPAPPWQGRSVASSKLWMLEF |
| 1096 | PRRC1 | PVRPSAPLPFVPPPAVPSVPPLVTSMPPPVSPSTAAAFGNPP |
| | | VSHFPPSTSAPNTLLPAPPS |
| 1097 | TMPRSS13_0 | SHGNASPARTPSAGASPAQASPAGTPPGRASPAQASPAQA |
| | | SPAGTPPGRASPAQASPAGTPP |
| 1098 | TMPRSS13_1 | SPARTPSAGASPAQASPAGTPPGRASPAQASPAQASPAGTP |
| | | PGRASPAQASPAGTPPGRASP |
| 1099 | TMPRSS13_2 | PSAGASPAQASPAGTPPGRASPAQASPAQASPAGTPPGRA |
| | | SPAQASPAGTPPGRASPGRASP |
| 1100 | TMPRSS13_3 | SPAGTPPGRASPAQASPAQASPAGTPPGRASPAQASPAGTP |
| | | PGRASPGRASPAQASPAQASP |
| 1101 | TMPRSS13_4 | PPGRASPAQASPAQASPAGTPPGRASPAQASPAGTPPGRAS |
| | | PGRASPAQASPAQASPARASP |
| 1102 | TMPRSS13_5 | SPAQASPAGTPPGRASPAQASPAGTPPGRASPGRASPAQAS |
| | | PAQASPARASPALASLSRSSS |
| 1103 | TMPRSS13_6 | SPAGTPPGRASPAQASPAGTPPGRASPGRASPAQASPAQAS |
| | | PARASPALASLSRSSSGRSSS |
| 1104 | TMPRSS13_7 | PPGRASPAQASPAGTPPGRASPGRASPAQASPAQASPARA |
| | | SPALASLSRSSSGRSSSARSAS |
| 1105 | TMPRSS13_8 | SPAQASPAGTPPGRASPGRASPAQASPAQASPARASPALAS |
| | | LSRSSSGRSSSARSASVTTSP |
| 1106 | TMPRSS13_9 | SPAGTPPGRASPGRASPAQASPAQASPARASPALASLSRSS |
| | | SGRSSSARSASVTTSPTRVYL |
| 1107 | TMPRSS13_10 | SLSRSSSGRSSSARSASVTTSPTRVYLVRATPVGAVPIRSSP |
| | | ARSAPATRATRESPGTSLPK |
| 1108 | TMPRSS13_11 | SSARSASVTTSPTRVYLVRATPVGAVPIRSSPARSAPATRA |
| | | TRESPGTSLPKFTWREGQKQL |
| 1109 | SUPT5H_0 | THSPASYHPTPSPMAYQASPSPSPVGYSPMTPGAPSPGGY |
| | | NPHTPGSGIEQNSSDWVTTDIQ |
| 1110 | SUPT5H_1 | SYHPTPSPMAYQASPSPSPVGYSPMTPGAPSPGGYNPHTP |
| | | GSGIEQNSSDWVTTDIQVKVRD |
| 1111 | SOX5 | ATAGVVYPGAIAMAGMPSPHLPSEHSSVSSSPEPGMPVIQS |
| | | TYGVKGEEPHIKEEIQAEDIN |
| 1112 | AIRE | TWRCSSCLQATVQEVQPRAEEPRPQEPPVETPLPPGLRSA |
| | | GEEVRGPPGEPLAGMDTTLVYK |
| 1113 | SEC16A_0 | HGGHPHGNMPGLDRPLSRQNPHDGVVTPAASPSLPQPGL |
| | | QMPGQWGPVQGGPQPSGQHRSPC |
| 1114 | SEC16A_1 | PDGPLASPARVPMFPVPLPPGPLEPGPGCVTPGPALGFLEP |
| | | SGPGLPPGVPPLQERRHLLQE |
| 1115 | SEC16A_2 | GTQRSEPALAPADFVAPLAPLPIPSNLFVPTPDAEEPQLPD |
| | | GTGREGPAAARGLANPEPAPE |
| 1116 | MYO18B | GDVLLMVAKLDPDSAKPEKTHPHDAPPCKTSPPATDTGK |
| | | EKKGETSRTPCGSQASTEILAPK |
| 1117 | NAV2 | NSVKVNPAAQPVSSPAQTSLQPGAKYPDVASPTLRRLFGG |
| | | KPTKQVPIATAENMKNSVVISN |
| 1118 | TCF7L2_0 | LEEAAKRQDGGLFKGPPYPGYPFIMIPDLTSPYLPNGSLSP |
| | | TARTLHFQSGSTHYSAYKTIE |
| 1119 | TCF7L2_1 | HFTPGNPPPHLPADVDPKTGIPRPPHPPDISPYYPLSPGTVG |
| | | QIPHPLGWLVPQQGQPVYPI |
| 1120 | CHEK2 | TLSSLETVSTQELYSIPEDQEPEDQEPEEPTPAPWARLWAL |
| | | QDGFANLECVNDNYWFGRDKS |
| 1121 | IL15RA_0 | CIRDPALVHQRPAPPSTVTTAGVTPQPESLSPSGKEPAASSP |
| | | SSNNTAATTAAIVPGSQLMP |
| 1122 | IL15RA_1 | RPAPPSTVTTAGVTPQPESLSPSGKEPAASSPSSNNTAATT |
| | | AAIVPGSQLMPSKSPSTGTTE |
| 1123 | UHRF2 | LNDIIQLLVRPDPDHLPGTSTQIEAKPCSNSPPKVKKAPRV |
| | | GPSNQPSTSARARLIDPGFGI |
| 1124 | PDLIM2_0 | DSSLEVLATRFQGSVRTYTESQSSLRSSYSSPTSLSPRAGSP |
| | | FSPPPSSSSLTGEAAISRSF |
| 1125 | PDLIM2_1 | VLATRFQGSVRTYTESQSSLRSSYSSPTSLSPRAGSPFSPPP |
| | | SSSSLTGEAAISRSFQSLAC |
| 1126 | PDLIM2_2 | FQGSVRTYTESQSSLRSSYSSPTSLSPRAGSPFSPPPSSSSLT |
| | | GEAAISRSFQSLACSPGLP |
| 1127 | PNPLA6 | HNYLGLTNELFSHEIQPLRLFPSPGLPTRTSPVRGSKRMVS |
| | | TSATDEPRETPGRPPDPTGAP |
| 1128 | GP1BA_0 | TQESTKEQTTFPPRWTPNFTLHMESITFSKTPKSTTEPTPSP |
| | | TTSEPVPEPAPNMTTLEPTP |
| 1129 | GP1BA_1 | TPKSTTEPTPSPTTSEPVPEPAPNMTTLEPTPSPTTPEPTSEP |
| | | APSPTTPEPTSEPAPSPTT |
| 1130 | GP1BA_2 | TEPTPSPTTSEPVPEPAPNMTTLEPTPSPTTPEPTSEPAPSPT |
| | | TPEPTSEPAPSPTTPEPTS |
| 1131 | GP1BA_3 | EPVPEPAPNMTTLEPTPSPTTPEPTSEPAPSPTTPEPTSEPAP |
| | | SPTTPEPTSEPAPSPTTPE |
| 1132 | GP1BA_4 | PEPAPNMTTLEPTPSPTTPEPTSEPAPSPTTPEPTSEPAPSPT |
| | | TPEPTSEPAPSPTTPEPTP |
| 1133 | GP1BA_5 | EPTPSPTTPEPTSEPAPSPTTPEPTSEPAPSPTTPEPTSEPAPS |
| | | PTTPEPTPIPTIATSPTI |
| 1134 | GP1BA_6 | PSPTTPEPTSEPAPSPTTPEPTSEPAPSPTTPEPTSEPAPSPTT |
| | | PEPTPIPTIATSPTILVS |
| 1135 | GP1BA_7 | EPAPSPTTPEPTSEPAPSPTTPEPTSEPAPSPTTPEPTPIPTIAT |
| | | SPTILVSATSLITPKST |
| 1136 | ADAMTS7 | FLPEEDTPIGAPDLGLPSLSWPRVSTDGLQTPATPESQNDF |
| | | PVGKDSQSQLPPPWRDRTNEV |
| 1137 | TRIB1 | LDADDAAAVAAKCPRLSECSSPPDYLSPPGSPCSPQPPPAA |
| | | PGAGGGSGSAPGPSRIADYLL |
| 1138 | GMEB1 | QNVVLMPVSTPKPPKRPRLQRPASTTVLSPSPPVQQPQFTV |
| | | ISPITITPVGQSFSMGNIPVA |
| 1139 | RNF213 | LPRGLQVGQPNLVVCGHSEVLPAALAVYMQTPSQPLPTY |
| | | DEVLLCTPATTFEEVALLLRRCL |
| 1140 | IFI16_0 | ALSRKRKKEVDATSPAPSTSSTVKTEGAEATPGAQNPKTV |
| | | AKCQVTPRRNVLQKRPVIVKVL |
| 1141 | IFI16_1 | LKEGSHFPGPFMTSIGPAESHPHTPQMPPSTPSSSFLTTLKP |
| | | RLKTEPEEVSIEDSAQSDLK |
| 1142 | KDM2A_0 | KAQKRKMEESDEEAVQAKVLRPLRSCDEPLTPPPHSPTSM |
| | | LQLIHDPVSPRGMVTRSSPGAG |
| 1143 | KDM2A_1 | KMEESDEEAVQAKVLRPLRSCDEPLTPPPHSPTSMLQLIHD |
| | | PVSPRGMVTRSSPGAGPSDHH |
| 1144 | NRK | ASAILYAGFVEVPEESPKQPSEVNVNPLYVSPACKKPLIHM |
| | | YEKEFTSEICCGSLWGVNLLL |
| 1145 | CGNL1 | SNWLKTLTEEGINNKKPWTCFPKPSNSQPTSPSLEDPAKSG |
| | | VTAIRLCSSVVIEDPKKQTSV |
| 1146 | DMTN | STSPPPSPEVWADSRSPGIISQASAPRTTGTPRTSLPHFHHP |
| | | ETSRPDSNIYKKPPIYKQRE |
| 1147 | PABPC4 | TAVQNLAPRAAVAAAAPRAVAPYKYASSVRSPHPAIQPL |
| | | QAPQPAVHVQGQEPLTASMLAAA |
| 1148 | E2F1_0 | AAQDASAPPAPTGPAAPAAGPCDPDLLLFATPQAPRPTPS |
| | | APRPALGRPPVKRRLDLETDHQ |
| 1149 | E2F1_1 | PPAPTGPAAPAAGPCDPDLLLFATPQAPRPTPSAPRPALGR |
| | | PPVKRRLDLETDHQYLAESSG |
| 1150 | KPRP | GASCPELRPHVEPRPLPSFCPPRRLDQCPESPLQRCPPPAPR |
| | | PRLRPEPCISLEPRPRPLPR |
| 1151 | AGER | EEVQLVVEPEGGAVAPGGTVTLTCEVPAQPSPQIHWMKD |
| | | GVPLPLPPSPVLILPEIGPQDQG |
| 1152 | SIK3 | AAGAGTGGAGPAGRLLPPPAPGSPAAPAAVSPAAGQPRPP |
| | | APASRGPMPARIGYYEIDRTIG |
| 1153 | TAF4B | GETSGAAICLPSVKPVVSSAGTTSDKPVIGTPVQIKLAQPG |
| | | PVLSQPAGIPQAVQVKQLVVQ |
| 1154 | AKNA | PIMPYPPAAVYYAPAGPTSAQPAAKWPPTASPPPARRHRH |
| | | SIQLDLGDLEELNKALSRAVQA |
| 1155 | NUP62 | STAQPSGFNIGSAGNSAQPTAPATLPFTPATPAATTAGATQ |
| | | PAAPTPTATITSTGPSLFASI |
| 1156 | ARHGAP33_0 | RAGGGGRDAPEAAAQSPCSVPSQVPTPGFFSPAPRECLPPF |
| | | LGVPKPGLYPLGPPSFQPSSP |
| 1157 | ARHGAP33_1 | TRSWSPFRSMPPDRLNASYGMLGQSPPLHRSPDFLLSYPP |
| | | APSCFPPDHLGYSAPQHPARRP |
| 1158 | ARHGAP33_2 | PARRPTPPEPLYVNLALGPRGPSPASSSSSSPPAHPRSRSDP |
| | | GPPVPRLPQKQRAPWGPRTP |
| 1159 | TEAD2 | DVKPFSQTPFTLSLTPPSTDLPGYEPPQALSPLPPPTPSPPA |
| | | WQARGLGTARLQLVEFSAFV |
| 1160 | TP53BP1_0 | EEGGEPFQKKLQSGEPVELENPPLLPESTVSPQASTPISQST |
| | | PVFPPGSLPIPSQPQFSHDI |
| 1161 | TP53BP1_1 | PFQKKLQSGEPVELENPPLLPESTVSPQASTPISQSTPVFPP |
| | | GSLPIPSQPQFSHDIFIPSP |
| 1162 | PPP1R13B_0 | LERRKEGSLPRPSAGLPSRQRPTLLPATGSTPQPGSSQQIQQ |
| | | RISVPPSPTYPPAGPPAFPA |
| 1163 | PPP1R13B_1 | PSESTEKEPEQDGPAAPADGSTVESLPRPLSPTKLTPIVHSP |
| | | LRYQSDADLEALRRKLANAP |
| 1164 | PPP1R13B_2 | EKEPEQDGPAAPADGSTVESLPRPLSPTKLTPIVHSPLRYQ |
| | | SDADLEALRRKLANAPRPLKK |
| 1165 | PPP1R13B_3 | QDGPAAPADGSTVESLPRPLSPTKLTPIVHSPLRYQSDADL |
| | | EALRRKLANAPRPLKKRSSIT |
| 1166 | EML3_0 | QEMELVKAALAEALRLLRLQVPPSSLQGSGTPAPPGDSLA |
| | | APPGLPPTCTPSLVSRGTQTET |
| 1167 | EML3_1 | SEGGGSSSSGAGSPGPPGILRPLQPPQRADTPRRNSSSSSSP |
| | | SERPRQKLSRKAISSANLLV |
| 1168 | ZDHHC8 | SLSYDSLLNPGSPGGHACPAHPAVGVAGYHSPYLHPGAT |
| | | GDPPRPLPRSFSPVLGPRPREPS |
| 1169 | HIF3A | QLNASEQLPRAYHRPLGAVPRPRARSFHGLSPPALEPSLLP |
| | | RWGSDPRLSCSSPSRGDPSAS |
| 1170 | ZNF385A_0 | ARRVKGIEAAKTRGREPGVREPGDPAPPGSTPTNGDGVAP |
| | | RPVSMENGLGPAPGSPEKQPGS |
| 1171 | ZNF385A_1 | TFSKELPKSLAGGLLPSPLAVAAVMAAAAGSPLSLRPAPA |
| | | APLLQGPPITHPLLHPAPGPIR |
| 1172 | VASN_0 | ATTTTATVPTTRPVVREPTALSSSLAPTWLSPTEPATEAPSP |
| | | PSTAPPTVGPVPQPQDCPPS |
| 1173 | VASN_1 | TRPVVREPTALSSSLAPTWLSPTEPATEAPSPPSTAPPTVGP |
| | | VPQPQDCPPSTCLNGGTCHL |
| 1174 | MYRF_0 | CFPDISAPASSASYSHGQPAMPGSSGVHHLSPPGGGPSPGR |
| | | HGPLPPPGYGTPLNCNNNNGM |
| 1175 | MYRF_1 | PTRAPSPPWPPQGPLSPGPGSLPLSIARVQTPPWHPPGAPSP |
| | | GLLQDSDSLSGSYLDPNYQS |
| 1176 | MAP2K7 | RRRIDLNLDISPQRPRPTLQLPLANDGGSRSPSSESSPQHPT |
| | | PPARPRHMLGLPSTLFTPRS |
| 1177 | RORC | VVKTPPAGAQGADTLTYTLGLPDGQLPLGSSPDLPEASAC |
| | | PPGLLKASGSGPSYSNNLAKAG |
| 1178 | TRERF1 | SQLRSPRVLGDHLLLDPTHELPPYTPPPMLSPVRQGSGLFS |
| | | NVLISGHGPGAHPQLPLTPLT |
| 1179 | EIF4B | TSTTSSRNARRRESEKSLENETLNKEEDCHSPTSKPPKPDQ |
| | | PLKVMPAPPPKENAWVKRSSN |
| 1180 | MAP7D1_0 | RAGASLARGPQPDRTHPSAAVPVCPRSASASPLTPCSVTRS |
| | | VHRCAPAGERGERRKPNAGGS |
| 1181 | MAP7D1_1 | GPEDKSQSKRRASNEKESAAPASPAPSPAPSPTPAPPQKEQ |
| | | PPAETPTDAAVLTSPPAPAPP |
| 1182 | MAP7D1_2 | KESAAPASPAPSPAPSPTPAPPQKEQPPAETPTDAAVLTSPP |
| | | APAPPVTPSKPMAGTTDREE |
| 1183 | RAB11FIP5_0 | ASPHHSSSGEEKAKSSWFGLREAKDPTQKPSPHPVKPLSA |
| | | APVEGSPDRKQSRSSLSIALSS |
| 1184 | RAB11FIP5_1 | SWFGLREAKDPTQKPSPHPVKPLSAAPVEGSPDRKQSRSS |
| | | LSIALSSGLEKLKTVTSGSIQP |
| 1185 | RAD54L2 | LSEPRMFAPFPSPVLPSNLSRGMSIYPGYMSPHAGYPAGGL |
| | | LRSQVPPFDSHEVAEVGFSSN |
| 1186 | LZTS2 | CPSGTLSDSGRNSLSSLPTYSTGGAEPTTSSPGGHLPSHGS |
| | | GRGALPGPARGVPTGPSHSDS |
| 1187 | SH3BP1_0 | SGSPGTPQALPRRLVGSSLRAPTVPPPLPPTPPQPARRQSRR |
| | | SPASPSPASPGPASPSPVSL |
| 1188 | SH3BP1_1 | RLVGSSLRAPTVPPPLPPTPPQPARRQSRRSPASPSPASPGP |
| | | ASPSPVSLSNPAQVDLGAAT |
| 1189 | SH3BP1_2 | GSSLRAPTVPPPLPPTPPQPARRQSRRSPASPSPASPGPASPS |
| | | PVSLSNPAQVDLGAATAEG |
| 1190 | SH3BP1_3 | SLRAPTVPPPLPPTPPQPARRQSRRSPASPSPASPGPASPSPV |
| | | SLSNPAQVDLGAATAEGGA |
| 1191 | SH3BP1_4 | LPPTPPQPARRQSRRSPASPSPASPGPASPSPVSLSNPAQVD |
| | | LGAATAEGGAPEAISGVPTP |
| 1192 | L3MBTL1 | DHPDIHPAGWCSKTGHPLQPPLGPREPSSASPGGCPPLSYR |
| | | SLPHTRTSKYSFHHRKCPTPG |
| 1193 | NBEAL2_0 | ARQAGWQDVLTRLYVLEAATAGSPPPSSPESPTSPKPAPP |
| | | KPPTESPAEPSDVFLPSEAPCP |
| 1194 | NBEAL2_1 | AGWQDVLTRLYVLEAATAGSPPPSSPESPTSPKPAPPKPPT |
| | | ESPAEPSDVFLPSEAPCPDPD |
| 1195 | NBEAL2_2 | LEAATAGSPPPSSPESPTSPKPAPPKPPTESPAEPSDVFLPSE |
| | | APCPDPDGFYHALSPFCTP |
| 1196 | TP53 | EQWFTEDPGPDEAPRMPEAAPPVAPAPAAPTPAAPAPAPS |
| | | WPLSSSVPSQKTYQGSYGFRLG |
| 1197 | RGL3 | LSAKLAREKSSSPSGSPGDPSSPTSSVSPGSPPSSPRSRDAP |
| | | AGSPPASPGPQGPSTKLPLS |
| 1198 | PRG4_0 | TPKAETTTKGPALTTPKEPTPTTPKEPASTTPKEPTPTTIKS |
| | | APTTPKEPAPTTTKSAPTTP |
| 1199 | PRG4_1 | TTTKGPALTTPKEPTPTTPKEPASTTPKEPTPTTIKSAPTTPK |
| | | EPAPTTTKSAPTTPKEPAP |
| 1200 | PRG4_2 | PKEPTPTTPKEPASTTPKEPTPTTIKSAPTTPKEPAPTTTKSA |
| | | PTTPKEPAPTTTKEPAPTT |
| 1201 | PRG4_3 | TPKEPTPTTIKSAPTTPKEPAPTTTKSAPTTPKEPAPTTTKEP |
| | | APTTPKEPAPTTTKEPAPT |
| 1202 | PRG4_4 | TTPKEPAPTTPKKPAPTTPKEPAPTTPKEPTPTTPKEPAPTT |
| | | KEPAPTTPKEPAPTAPKKPA |
| 1203 | PRG4_5 | KEPAPTTPKKPAPTTPKEPAPTTPKEPTPTTPKEPAPTTKEP |
| | | APTTPKEPAPTAPKKPAPTT |
| 1204 | PRG4_6 | PKEPAPTTPKEPTPTTPKEPAPTTKEPAPTTPKEPAPTAPKK |
| | | PAPTTPKEPAPTTPKEPAPT |
| 1205 | PRG4_7 | KEPAPTTPKETAPTTPKGTAPTTLKEPAPTTPKKPAPKELA |
| | | PTTTKEPTSTTSDKPAPTTPK |
| 1206 | PRG4_8 | KEPAPTTPKEPAPTTPKGTAPTTLKEPAPTTPKKPAPKELA |
| | | PTTTKGPTSTTSDKPAPTTPK |
| 1207 | NHS | AGLASPSSGYSSQSETPTSSFPTAFFSGPLSPGGSKRKPKVP |
| | | ERKSSLQQPSLKDGTISLSK |
| 1208 | TNK2_0 | SAQTAEIFQALQQECMRQLQAPAGSPAPSPSPGGDDKPQV |
| | | PPRVPIPPRPTRPHVQLSPAPP |
| 1209 | TNK2_1 | PIPPRPTRPHVQLSPAPPGEEETSQWPGPASPPRVPPREPLS |
| | | PQGSRTPSPLVPPGSSPLPP |
| 1210 | TNK2_2 | STHYYLLPERPSYLERYQRFLREAQSPEEPTPLPVPLLLPPP |
| | | STPAPAAPTATVRPMPQAAL |
| 1211 | TNK2_3 | LERYQRFLREAQSPEEPTPLPVPLLLPPPSTPAPAAPTATVR |
| | | PMPQAALDPKANFSTNNSNP |
| 1212 | KMT2D_0 | KPLGKAGVQLEPQLEAPLNEEMPLLPPPEESPLSPPPEESPT |
| | | SPPPEASRLSPPPEELPASP |
| 1213 | KMT2D_1 | LEPQLEAPLNEEMPLLPPPEESPLSPPPEESPTSPPPEASRLS |
| | | PPPEELPASPLPEALHLSR |
| 1214 | KMT2D_2 | PEASRLSPPPEELPASPLPEALHLSRPLEESPLSPPPEESPLSP |
| | | PPESSPFSPLEESPLSPP |
| 1215 | KMT2D_3 | PESSPFSPLEESPLSPPEESPPSPALETPLSPPPEASPLSPPFEE |
| | | SPLSPPPEELPTSPPPE |
| 1216 | KMT2D_4 | PPEESPPSPALETPLSPPPEASPLSPPFEESPLSPPPEELPTSPP |
| | | PEASRLSPPPEESPMSP |
| 1217 | KMT2D_5 | FEESPLSPPPEELPTSPPPEASRLSPPPEESPMSPPPEESPMSP |
| | | PPEASRLFPPFEESPLSP |
| 1218 | KMT2D_6 | PEELPTSPPPEASRLSPPPEESPMSPPPEESPMSPPPEASRLFP |
| | | PFEESPLSPPPEESPLSP |
| 1219 | KMT2D_7 | PEESPMSPPPEESPMSPPPEASRLFPPFEESPLSPPPEESPLSP |
| | | PPEASRLSPPPEDSPMSP |
| 1220 | KMT2D_8 | PEESPMSPPPEASRLFPPFEESPLSPPPEESPLSPPPEASRLSP |
| | | PPEDSPMSPPPEESPMSP |
| 1221 | KMT2D_9 | FEESPLSPPPEESPLSPPPEASRLSPPPEDSPMSPPPEESPMSP |
| | | PPEVSRLSPLPVVSRLSP |
| 1222 | KMT2D_10 | PEESPLSPPPEASRLSPPPEDSPMSPPPEESPMSPPPEVSRLSP |
| | | LPVVSRLSPPPEESPLSP |
| 1223 | KMT2D_11 | PEESPMSPPPEVSRLSPLPVVSRLSPPPEESPLSPPPEESPTSP |
| | | PPEASRLSPPPEDSPTSP |
| 1224 | KMT2D_12 | PEVSRLSPLPVVSRLSPPPEESPLSPPPEESPTSPPPEASRLSP |
| | | PPEDSPTSPPPEDSPASP |
| 1225 | KMT2D_13 | PEESPLSPPPEESPTSPPPEASRLSPPPEDSPTSPPPEDSPASPP |
| | | PEDSLMSLPLEESPLLP |
| 1226 | KMT2D_14 | PEESPTSPPPEASRLSPPPEDSPTSPPPEDSPASPPPEDSLMSL |
| | | PLEESPLLPLPEEPQLCP |
| 1227 | KMT2D_15 | PEDSPTSPPPEDSPASPPPEDSLMSLPLEESPLLPLPEEPQLC |
| | | PRSEGPHLSPRPEEPHLSP |
| 1228 | KMT2D_16 | GEPALSEPGEPPLSPLPEELPLSPSGEPSLSPQLMPPDPLPPP |
| | | LSPIITAAAPPALSPLGEL |
| 1229 | KMT2D_17 | ILETPISPPPEANCTDPEPVPPMILPPSPGSPVGPASPILMEPL |
| | | PPQCSPLLQHSLVPQNSP |
| 1230 | KMT2D_18 | SPILMEPLPPQCSPLLQHSLVPQNSPPSQCSPPALPLSVPSPL |
| | | SPIGKVVGVSDEAELHEME |
| 1231 | KMT2D_19 | DTAPLDGIDAPGSQPEPGQTPGSLASELKGSPVLLDPEELA |
| | | PVTPMEVYPECKQTAGQGSPC |
| 1232 | KMT2D_20 | CALPPRSLPSDPFSRVPASPQSQSSSQSPLTPRPLSAEAFCPS |
| | | PVTPRFQSPDPYSRPPSRP |
| 1233 | KMT2D_21 | FSRVPASPQSQSSSQSPLTPRPLSAEAFCPSPVTPRFQSPDP |
| | | YSRPPSRPQSRDPFAPLHKP |
| 1234 | KMT2D_22 | VPASPQSQSSSQSPLTPRPLSAEAFCPSPVTPRFQSPDPYSR |
| | | PPSRPQSRDPFAPLHKPPRP |
| 1235 | KMT2D_23 | QSQSSSQSPLTPRPLSAEAFCPSPVTPRFQSPDPYSRPPSRPQ |
| | | SRDPFAPLHKPPRPQPPEV |
| 1236 | KMT2D_24 | GAGPRPQGPPRLPAPPGALSTGPVLGPVHPTPPPSSPQEPK |
| | | RPSQLPSPSSQLPTEAQLPPT |
| 1237 | KMT2D_25 | PQGPPRLPAPPGALSTGPVLGPVHPTPPPSSPQEPKRPSQLP |
| | | SPSSQLPTEAQLPPTHPGTP |
| 1238 | KMT2D_26 | ALSTGPVLGPVHPTPPPSSPQEPKRPSQLPSPSSQLPTEAQL |
| | | PPTHPGTPKPQGPTLEPPPG |
| 1239 | KMT2D_27 | YTYNVSNLDVRQLSAPPPEEPSPPPSPLAPSPASPPTEPLVE |
| | | LPTEPLAEPPVPSPLPLASS |
| 1240 | ARHGAP32 | RFYSGDQPPSYLGASVDKLHHPLEFADKSPTPPNLPSDKIY |
| | | PPSGSPEENTSTATMTYMTTT |
| 1241 | ZNF652_0 | EKPYPCDVCGQRFRFSNMLKAHKEKCFRVTSPVNVPPAV |
| | | QIPLTTSPATPVPSVVNTATTPT |
| 1242 | ZNF652_1 | SNMLKAHKEKCFRVTSPVNVPPAVQIPLTTSPATPVPSVV |
| | | NTATTPTPPINMNPVSTLPPRP |
| 1243 | TNS2_0 | SYGGAVPSYCPAYGRVPHSCGSPGEGRGYPSPGAHSPRAG |
| | | SISPGSPPYPQSRKLSYEIPTE |
| 1244 | TNS2_1 | ASSELSGPSTPLHTSSPVQGKESTRRQDTRSPTSAPTQRLSP |
| | | GEALPPVSQAGTGKAPELPS |
| 1245 | TNS2_2 | PGEALPPVSQAGTGKAPELPSGSGPEPLAPSPVSPTFPPSSP |
| | | SDWPQERSPGGHSDGASPRS |
| 1246 | TNS2_3 | ALPPVSQAGTGKAPELPSGSGPEPLAPSPVSPTFPPSSPSDW |
| | | PQERSPGGHSDGASPRSPVP |
| 1247 | TNS2_4 | SPRSPVPTTLPGLRHAPWQGPRGPPDSPDGSPLTPVPSQMP |
| | | WLVASPEPPQSSPTPAFPLAA |
| 1248 | TNS2_5 | SLSALVSQHSISPISLPCCLRIPSKDPLEETPEAPVPTNMSTA |
| | | ADLLRQGAACSVLYLTSVE |
| 1249 | ARHGAP27_0 | LPSPVWETHTDAGTGRPYYYNPDTGVTTWESPFEAAEGA |
| | | ASPATSPASVDSHVSLETEWGQY |
| 1250 | ARHGAP27_1 | WEDEAENEPEEELEMQPGLSPGSPGDPRPPTPETDYPESLT |
| | | SYPEEDYSPVGSFGEPGPTSP |
| 1251 | FOXL1 | RSAEAQPEAGSGAGGSGPAISRLQAAPAGPSPLLDGPSPPA |
| | | PLHWPGTASPNEDAGDAAQGA |
| 1252 | TMEM132E | GPGGGEDEARGAGPPGSALPAPEAPGPGTASPVVPPTEDF |
| | | LPLPTGFLQVPRGLTDLEIGMY |
| 1253 | SOS1 | DYLFNKSLEIEPRNPKPLPRFPKKYSYPLKSPGVRPSNPRPG |
| | | TMRHPTPLQQEPRKISYSRI |
| 1254 | CRAMP1 | PSPRPGPGLLLDVCTKDLADAPAEELQEKGSPAGPPPSQG |
| | | QPAARPPKEVPASRLAQQLREE |
| 1255 | PIAS1 | EEPSAKRTCPSLSPTSPLNNKGILSLPHQASPVSRTPSLPAV |
| | | DTSYINTSLIQDYRHPFHMT |
| 1256 | PPP1R15B | AGDIPGNTQESTEEKIELLTTEVPLALEEESPSEGCPSSEIPM |
| | | EKEPGEGRISVVDYSYLEG |
| 1257 | JPH2_0 | LQEILENSESLLEPPDRGAGAAGLPQPPRESPQLHERETPRP |
| | | EGGSPSPAGTPPQPKRPRPG |
| 1258 | JPH2_1 | EVSGSESAPSSPATAPLQAPTLRGPEPARETPAKLEPKPIIP |
| | | KAEPRAKARKTEARGLTKAG |
| 1259 | PPFIBP2 | EEPEGGFSKWNATNKDPEELFKQEMPPRCSSPTVGPPPLP |
| | | QKSLETRAQKKLSCSLEDLRSE |
| 1260 | LPP_0 | IDSLTSILADLECSSPYKPRPPQSSTGSTASPPVSTPVTGHK |
| | | RMVIPNQPPLTATKKSTLKP |
| 1261 | LPP_1 | SILADLECSSPYKPRPPQSSTGSTASPPVSTPVTGHKRMVIP |
| | | NQPPLTATKKSTLKPQPAPQ |
| 1262 | PMEL | QAVPSGEGDAFELTVSCQGGLPKEACMEISSPGCQPPAQR |
| | | LCQPVLPSPACQLVLHQILKGG |
| 1263 | ITSN2 | SIAMKLIKLKLQGQQLPVVLPPIMKQPPMFSPLISARFGMG |
| | | SMPNLSIPQPLPPAAPITSLS |
| 1264 | CSTF2 | EVRGMEARGMDTRGPVPGPRGPIPSGMQGPSPINMGAVV |
| | | PQGSRQVPVMQGTGMQGASIQGG |
| 1265 | BCL9L_0 | LTISINQMGSPGMGHLKSPTLSQVHSPLVTSPSANLKSPQT |
| | | PSQMVPLPSANPPGPLKSPQV |
| 1266 | BCL9L_1 | PGMGHLKSPTLSQVHSPLVTSPSANLKSPQTPSQMVPLPSA |
| | | NPPGPLKSPQVLGSSLSVRSP |
| 1267 | ZNF142 | SFKQQRGLSTHLLKKCPVLLRKNKGLPRPDSPIPLQPVLPG |
| | | TQASEDTESGKPPPASQEAEL |
| 1268 | MED13L_0 | LNTPQMNTPVTLNSAAPASNSGAGVLPSPATPRFSVPTPRT |
| | | PRTPRTPRGGGTASGQGSVKY |
| 1269 | MED13L_1 | TLNSAAPASNSGAGVLPSPATPRFSVPTPRTPRTPRTPRGG |
| | | GTASGQGSVKYDSTDQGSPAS |
| 1270 | MED13L_2 | LYAQVCRHHLAPYLATLQLDSSLLIPPKYQTPPAAAQGQA |
| | | TPGNAGPLAPNGSAAPPAGSAF |
| 1271 | MED13L_3 | APYLATLQLDSSLLIPPKYQTPPAAAQGQATPGNAGPLAP |
| | | NGSAAPPAGSAFNPTSNSSSTN |
| 1272 | MASTL | PNQIKSGTPYRTPKSVRRGVAPVDDGRILGTPDYLAPELLL |
| | | GRAHGPAVDWWALGVCLFEFL |
| 1273 | SAMD11 | QGLAQHREGAAPAAAPSFSERELPQPPPLLSPQNAPHVAL |
| | | GPHLRPPFLGVPSALCQTPGYG |
| 1274 | BCORL1 | APVPTPVLAPMPASTPPAAPAPPSVPMPTPTPSSGPPSTPTL |
| | | IPAFAPTPVPAPTPAPIFTP |
| 1275 | SETD1B_0 | RTKLLFLREPDSDTELQMEGSPISSSSSQLSPLAPFGTNSQP |
| | | GFRGPTPPSSRPSSTGLEDI |
| 1276 | SETD1B_1 | HDLEVEPEPPMMLPLPLQPPLPPPRPPRPPSPPPEPETTDAS |
| | | HPSVPPEPLAEDHPPHTPGL |
| 1277 | SETD1B_2 | TEEYMELAKSRGPWRRPPKKRHEDLVPPAGSPELSPPQPL |
| | | FRPRSEFEEMTILYDIWNGGID |
| 1278 | ZCCHC8 | GSQSSESFQFQPPLPPDTPPLPRGTPPPVFTPPLPKGTPPLTP |
| | | SDSPQTRTASGAVDEDALT |
| 1279 | IKBKG | RKRHVEVSQAPLPPAPAYLSSPLALPSQRRSPPEEPPDFCCP |
| | | KCQYQAPDMDTLQIHVMECI |
| 1280 | LAS1L | ARRGWRLFNCSASLDWPRMVESCLGSPCWASPQLLRIIFK |
| | | AMGQGLPDEEQEKLLRICSIYT |
| 1281 | PDZD4_0 | PEKSDKDSTSAYNTGESCRSTPLLVEPLPESPLRRAMAGNS |
| | | NLNRTPPGPAVATPAKAAPPP |
| 1282 | PDZD4_1 | LVEPLPESPLRRAMAGNSNLNRTPPGPAVATPAKAAPPPG |
| | | SPAKFRSLSRDPEAGRRQHAEE |
| 1283 | PDZD4_2 | RRAMAGNSNLNRTPPGPAVATPAKAAPPPGSPAKFRSLSR |
| | | DPEAGRRQHAEERGRRNPKTGL |
| 1284 | ZNF106 | SAASFEVVRQCPTAEKPEQEHTPNKMPSLKSPLLPCPATKS |
| | | LSQKQDPKNISKNTKTNFFSP |
| 1285 | HNF1A | EEAFRHKLAMDTYSGPPPGPGPGPALPAHSSPGLPPPALSP |
| | | SKVHGVRYGQPATSETAEVPS |
| 1286 | CLASP2 | NTGNGTQSSMGSPLTRPTPRSPANWSSPLTSPTNTSQNTLS |
| | | PSAFDYDTENMNSEDIYSSLR |
| 1287 | KMT2B_0 | PVVSARSSRVIKTPRRFMDEDPPKPPKVEVSPVLRPPITTSP |
| | | PVPQEPAPVPSPPRAPTPPS |
| 1288 | KMT2B_1 | IKTPRRFMDEDPPKPPKVEVSPVLRPPITTSPPVPQEPAPVP |
| | | SPPRAPTPPSTPVPLPEKRR |
| 1289 | KMT2B_2 | EVSPVLRPPITTSPPVPQEPAPVPSPPRAPTPPSTPVPLPEKR |
| | | RSILREPTFRWTSLTRELP |
| 1290 | CIC_0 | PLVSPPFSVPVQNGAQPPSKIIQLTPVPVSTPSGLVPPLSPAT |
| | | LPGPTSQPQKVLLPSSTRI |
| 1291 | CIC_1 | PTAPESELEGQPTPPAPPPLPETWTPTARSSPPLPPPAEERTS |
| | | AKGPETMASKFPSSSSDWR |
| 1292 | CIC_2 | FQARYADIFPSKVCLQLKIREVRQKIMQAATPTEQPPGAE |
| | | APLPVPPPTGTAAAPAPTPSPA |
| 1293 | DCTN1_0 | GPSGSASAGELSSSEPSTPAQTPLAAPIIPTPVLTSPGAVPPL |
| | | PSPSKEEEGLRAQVRDLEE |
| 1294 | DCTN1_1 | ASAGELSSSEPSTPAQTPLAAPIIPTPVLTSPGAVPPLPSPSK |
| | | EEEGLRAQVRDLEEKLETL |
| 1295 | EPN1_0 | PWGGPAPTPASGDPWRPAAPAGPSVDPWGGTPAPAAGEG |
| | | PTPDPWGSSDGGVPVSGPSASDP |
| 1296 | EPN1_1 | SGDPWRPAAPAGPSVDPWGGTPAPAAGEGPTPDPWGSSD |
| | | GGVPVSGPSASDPWTPAPAFSDP |
| 1297 | EPN1_2 | GSSDGGVPVSGPSASDPWTPAPAFSDPWGGSPAKPSTNGT |
| | | TAAGGFDTEPDEFSDFDRLRTA |
| 1298 | EPN1_3 | EVPARSPGAFDMSGVRGSLAEAVGSPPPAATPTPTPPTRKT |
| | | PESFLGPNAALVDLDSLVSRP |
| 1299 | EPN1_4 | DMSGVRGSLAEAVGSPPPAATPTPTPPTRKTPESFLGPNAA |
| | | LVDLDSLVSRPGPTPPGAKAS |
| 1300 | CEBPE | TAMHLPPTLAAPGQPLRVLKAPLATAAPPCSPLLKAPSPA |
| | | GPLHKGKKAVNKDSLEYRLRRE |
| 1301 | RFX4 | MKGEGSTAEVREEIILTEAAAPTPSPVPSFSPAKSATSVEVP |
| | | PPSSPVSNPSPEYTGLSTTG |
| 1302 | LPIN3 | PLGLPIQQTEAGADLQPDTEDPTLVGPPLHTPETEESKTQS |
| | | SGDMGLPPASKSWSWATLEVP |
| 1303 | RAPGEF1 | SQSTELLPDATDEEVAPPKPPLPGIRVVDNSPPPALPPKKR |
| | | QSAPSPTRVAVVAPMSRATSG |
| 1304 | SAMD4A | AYSSPSTTPEARRREPQAPRQPSLMGPESQSPDCKDGAAA |
| | | TGATATPSAGASGGLQPHQLSS |
| 1305 | MAST4_0 | NPQQREGSSPKHQDHTTDPKLLTCLGQNLHSPDLARPRCP |
| | | LPPEASPSREKPGLRESSERGP |
| 1306 | MAST4_1 | TTDPKLLTCLGQNLHSPDLARPRCPLPPEASPSREKPGLRE |
| | | SSERGPPTARSERSAARADTC |
| 1307 | PRRC2C | QTHKPVQNPLQTTSQSSKQPPPSIRLPSAQTPNGTDYVASG |
| | | KSIQTPQSHGTLTAELWDNKV |
| 1308 | PROP1 | MEAERRRQAEKPKKGRVGSNLLPERHPATGTPTTTVDSSA |
| | | PPCRRLPGAGGGRSRFSPQGGQ |
| 1309 | ARMC5_0 | RAQGGSFRSLRSWLISEGYATGPDDISPDWSPEQCPPEPME |
| | | PASPAPTPTSLRAPRTQRTPG |
| 1310 | ARMC5_1 | ADSLSCLQDLVSPTVSPAVPQAVPMDLDSPSPCLYEPLLGP |
| | | APVPAPDLHFLLDSGLQLPAQ |
| 1311 | CRYBG1_0 | SSPTKRKGRSRALEAVPAPPASGPRAPAKESPPKRVPDPSP |
| | | VTKGTAAESGEEAARAIPREL |
| 1312 | CRYBG1_1 | PTTVDTKDLPPTAMPKPQHTFSDSQSPAESSPGPSLSLSAP |
| | | APGDVPKDTCVQSPISSFPCT |
| 1313 | DLAT_0 | IIVEKEADISAFADYRPTEVTDLKPQVPPPTPPPVAAVPPTP |
| | | QPLAPTPSAPCPATPAGPKG |
| 1314 | DLAT_1 | AFADYRPTEVTDLKPQVPPPTPPPVAAVPPTPQPLAPTPSA |
| | | PCPATPAGPKGRVFVSPLAKK |
| 1315 | DLAT_2 | TEVTDLKPQVPPPTPPPVAAVPPTPQPLAPTPSAPCPATPA |
| | | GPKGRVFVSPLAKKLAVEKGI |
| 1316 | DLAT_3 | QVPPPTPPPVAAVPPTPQPLAPTPSAPCPATPAGPKGRVFV |
| | | SPLAKKLAVEKGIDLTQVKGT |
| 1317 | DENND2B_0 | ACRYPSHSSSRVLLKDRHPPAPSPQNPQDPSPDTSPPTCPF |
| | | KTASFGYLDRSPSACKRDAQK |
| 1318 | DENND2B_1 | NPVPKPKRTFEYEADKNPKSKPSNGLPPSPTPAAPPPLPSTP |
| | | APPVTRRPKKDMRGHRKSQS |
| 1319 | DENND2B_2 | EYEADKNPKSKPSNGLPPSPTPAAPPPLPSTPAPPVTRRPK |
| | | KDMRGHRKSQSRKSFEFEDAS |
| 1320 | PCDH12 | CEVGQSHKDVDKEAMMEAGWDPCLQAPFHLTPTLYRTL |
| | | RNQGNQGAPAESREVLQDTVNLLF |
| 1321 | SCARF2_0 | HTVEHGSPRTRDPTPRPPGLPEEATALAAPSPPRARARGRG |
| | | PGLLEPTDAGGPPRSAPEAAS |
| 1322 | SCARF2_1 | LGRAEVALGAQGPREKPAPPQKAKRSVPPASPARAPPATE |
| | | TPGPEKAATDLPAPETPRKKTP |
| 1323 | SCARF2_2 | QGPREKPAPPQKAKRSVPPASPARAPPATETPGPEKAATD |
| | | LPAPETPRKKTPIQKPPRKKSR |
| 1324 | IRAG1 | PGTRGHSQQEAAMPHIPEDEEPPGEPQAAQSPAGQGPPAA |
| | | GVSCSPTPTIVLTGDATSPEGE |
| 1325 | CAMSAP3_0 | SLASPYLPEGTSKPLSDRPTKAPVYMPHPETPSKPSPCLVG |
| | | EASKPPAPSEGSPKAVASSPA |
| 1326 | CAMSAP3_1 | YLPEGTSKPLSDRPTKAPVYMPHPETPSKPSPCLVGEASKP |
| | | PAPSEGSPKAVASSPAATNSE |
| 1327 | SP110_0 | QPPQPSCSPCAPRVSEPGTSSQQSDEILSESPSPSDPVLPLPA |
| | | LIQEGRSTSVTNDKLTSKM |
| 1328 | SP110_1 | DNLIPQIRDKEDPQEMPHSPLGSMPEIRDNSPEPNDPEEPQE |
| | | VSSTPSDKKGKKRKRCIWST |
| 1329 | COL6A2 | QKGKLGRIGPPGCKGDPGNRGPDGYPGEAGSPGERGDQG |
| | | GKGDPGRPGRRGPPGEIGAKGSK |
| 1330 | POLR1G | TCASAPQGTLRILEGPQQSLSGSPLQPIPASPPPQIPPGLRPR |
| | | FCAFGGNPPVTGPRSALAP |
| 1331 | USP54 | CSSSSSLPVIHDPSVFLLGPQLYLPQPQFLSPDVLMPTMAG |
| | | EPNRLPGTSRSVQQFLAMCDR |
| 1332 | FILIP1L | HTPGQPLHIKVTPDHVQNTATLEITSPTTESPHSYTSTAVIP |
| | | NCGTPKQRITILQNASITPV |
| 1333 | LITAF | GPYQAATGPSSAPSAPPSYEETVAVNSYYPTPPAPMPGPTT |
| | | GLVTGPDGKGMNPPSYYTQPA |
| 1334 | GLIS3 | HNPSSQLPPLTAVDAGAERFAPSAPSPHHISPRRVPAPSSIL |
| | | QRTQPPYTQQPSGSHLKSYQ |
| 1335 | CPLANE1 | ISQAYGLMNELLSESVQLPTLPQKPLPNKPSPTQSSSCQHC |
| | | PSPRGENQHGHSFLINRPGKV |
| 1336 | CNOT2_0 | ALGLPMRGMSNNTPQLNRSLSQGTQLPSHVTPTTGVPTMS |
| | | LHTPPSPSRGILPMNPRNMMNH |
| 1337 | CNOT2_1 | LTFIRAAETDPGMVHLALGSDLTTLGLNLNSPENLYPKFA |
| | | SPWASSPCRPQDIDFHVPSEYL |
| 1338 | CNOT2_2 | PGMVHLALGSDLTTLGLNLNSPENLYPKFASPWASSPCRP |
| | | QDIDFHVPSEYLTNIHIRDKLA |
| 1339 | CNOT2_3 | LALGSDLTTLGLNLNSPENLYPKFASPWASSPCRPQDIDFH |
| | | VPSEYLTNIHIRDKLAAIKLG |
| 1340 | USP19_0 | LRKRQSQRWGGLEAPAARVGGAKVAVPTGPTPLDSTPPG |
| | | GAPHPLTGQEEARAVEKDKSKAR |
| 1341 | USP19_1 | SQRWGGLEAPAARVGGAKVAVPTGPTPLDSTPPGGAPHP |
| | | LTGQEEARAVEKDKSKARSEDTG |
| 1342 | CNTFR | EFTIVKPDPPENVVARPVPSNPRRLEVTWQTPSTWPDPESF |
| | | PLKFFLRYRPLILDQWQHVEL |
| 1343 | MYO19 | QARYMADTFYTNAGCTLVALNPFKPVPQLYSPELMREYH |
| | | AAPQPQKLKPHVFTVGEQTYRNV |
| 1344 | NR4A1 | YGSPCSAPSPSTPSFQPPQLSPWDGSFGHFSPSQTYEGLRA |
| | | WTEQLPKASGPPQPPAFFSFS |
| 1345 | FAT4 | RSKSPQAMASHGSRPGSRLKQPIGQIPLESSPPVGLSIEEVE |
| | | RLNTPRPRNPSICSADHGRS |
| 1346 | CC2D1B | RRGRKINEDEIPPPVALGKRPLAPQEPANRSPETDPPAPPAL |
| | | ESDNPSQPETSLPGISAQPV |
| 1347 | GRB7_0 | LDLSPPHLSSSPEDLCPAPGTPPGTPRPPDTPLPEEVKRSQP |
| | | LLIPTTGRKLREEERRATSL |
| 1348 | GRB7_1 | LIPTTGRKLREEERRATSLPSIPNPFPELCSPPSQSPILGGPSS |
| | | ARGLLPRDASRPHVVKVY |
| 1349 | GRB7_2 | GRKLREEERRATSLPSIPNPFPELCSPPSQSPILGGPSSARGL |
| | | LPRDASRPHVVKVYSEDGA |
| 1350 | STPG1 | PGYYNPSDCTKVPKKTLFPKNPILNFSAQPSPLPPKPPFPGP |
| | | GQYEIVDYLGPRKHFISSAS |
| 1351 | TCOF1 | NPAAARAPSAKGTISAPGKVVTAAAQAKQRSPSKVKPPV |
| | | RNPQNSTVLARGPASVPSVGKAV |
| 1352 | ELF2_0 | PEFIHAAMRPDVITETVVEVSTEESEPMDTSPIPTSPDSHEP |
| | | MKKKKVGRKPKTQQSPISNG |
| 1353 | ELF2_1 | AAMRPDVITETVVEVSTEESEPMDTSPIPTSPDSHEPMKKK |
| | | KVGRKPKTQQSPISNGSPELG |
| 1354 | BRD4_0 | GRGRKETGTAKPGVSTVPNTTQASTPPQTQTPQPNPPPVQ |
| | | ATPHPFPAVTPDLIVQTPVMTV |
| 1355 | BRD4_1 | QATPHPFPAVTPDLIVQTPVMTVVPPQPLQTPPPVPPQPQP |
| | | PPAPAPQPVQSHPPIIAATPQ |
| 1356 | BRD4_2 | PQQPSRPSNRAAALPPKPARPPAVSPALTQTPLLPQPPMAQ |
| | | PPQVLLEDEEPPAPPLTSMQM |
| 1357 | MAP3K9 | DGALKPETLLASRSPSSNGLSPSPGAGMLKTPSPSRDPGEF |
| | | PRLPDPNVVFPPTPRRWNTQQ |
| 1358 | CBFA2T2 | RREENSFDRDTIAPEPPAKRVCTISPAPRHSPALTVPLMNP |
| | | GGQFHPTPPPLQHYTLEDIAT |
| 1359 | MYPN_0 | SEASSEAGVVTTRQTRPDSFQERFNGQATKTPEPSSPVKEP |
| | | PPVLAKPKLDSTQLQQLHNQV |
| 1360 | MYPN_1 | LLVSHPSVQTKSPGGLSIQNEPLPPGPTEPTPPPFTFSIPSGN |
| | | QFQPRCVSPIPVSPTSRIQ |
| 1361 | PTCHD3 | SATGPQWYQESQESESEGKQPPPGPLAPPKSPEPSGPLASE |
| | | QDAPLPEGDDAPPRPSMLDDA |
| 1362 | KDM6B | PPAPPSSCHQNTSGSFRRPESPRPRVSFPKTPEVGPGPPPGP |
| | | LSKAPQPVPPGVGELPARGP |
| 1363 | C2CD5 | GESGLVVRAIGTACTLDKLSSPAAFLPACNSPSKEMKEIPF |
| | | NEDPNPNTHSSGPSTPLKNQT |
| 1364 | SEC16B | GTTTENTFYQDFSGCQGYSEAPGYRSALWLTPEQTCLLQP |
| | | SPQQPFPLQPGSYPAGGGAGQT |
| 1365 | ARAP1_0 | AHTSPAPAPRPTPRPVPMKRHIFRSPPVPATPPEPLPTTTED |
| | | EGLPAAPPIPPRRSCLPPTC |
| 1366 | ARAP1_1 | NGGWHTSSLSLSLPSTIAAPHPMDGPPGGSTPVTPVIKAG |
| | | WLDKNPPQGSYIYQKRWVRLDT |
| 1367 | TRAPPC12 | EGDAGDLGRVRDEAEPGGEGDPGPEPAGTPSPSGEADGD |
| | | CAPEDAAPSSGGAPRQDAAREVP |
| 1368 | ACACA | ADVNLPAAQLQIAMGIPLYRIKDIRMMYGVSPWGDSPIDF |
| | | EDSAHVPCPRGHVIAARITSEN |
| 1369 | UBP1_0 | EDAVEHEQKKSSKRTLPADYGDSLAKRGSCSPWPDAPTA |
| | | YVNNSPSPAPTFTSPQQSTCSVP |
| 1370 | UBP1_1 | LPADYGDSLAKRGSCSPWPDAPTAYVNNSPSPAPTFTSPQ |
| | | QSTCSVPDSNSSSPNHQGDGAS |
| 1371 | DENND1A | AWSGSTLPSRPATPNVATPFTPQFSFPPAGTPTPFPQPPLNP |
| | | FVPSMPAAPPTLPLVSTPAG |
| 1372 | FAM193A_0 | GIMDPPVTDDIHIHQLPLQVDPAPDYLAERSPPSVSSASSGS |
| | | GSSSPITIQQHPRLILTDSG |
| 1373 | FAM193A_1 | SSEADDEEADGESSGEPPGAPKEDGVLGSRSPRTEESKADS |
| | | PPPSYPTQQAEQAPNTCECHV |
| 1374 | FAM193A_2 | LHLYPHIHGHVPLHTVPHLPRPLIHPTLYATPPFTHSKALPP |
| | | APVQNHTNKHQVFNASLQDH |
| 1375 | FAM193A_3 | FHGISKEDHRHSAPAAPRNSPTGLAPLPALSPAALSPAALS |
| | | PASTPHLANLAAPSFPKTATT |
| 1376 | FAM193A_4 | HSAPAAPRNSPTGLAPLPALSPAALSPAALSPASTPHLANL |
| | | AAPSFPKTATTTPGFVDTRKS |
| 1377 | SCYL3 | LNQLVFAEPVAVKSFLPYLLGPKKDHAQGETPCLLSPALF |
| | | QSRVIPVLLQLFEVHEEHVRMV |
| 1378 | QRICH1_0 | LTVHQPTEQPIQVQVQIQGQAPQSAAPSIQTPSLQSPSPSQL |
| | | QAAQIQVQHVQAAQQIQAAE |
| 1379 | QRICH1_1 | PTEQPIQVQVQIQGQAPQSAAPSIQTPSLQSPSPSQLQAAQI |
| | | QVQHVQAAQQIQAAEIPEEH |
| 1380 | TFPT_0 | TIVLEDEGSQGTDAPTPGNAENEPPEKETLSPPRRTPAPPEP |
| | | GSPAPGEGPSGRKRRRVPRD |
| 1381 | TFPT_1 | DEGSQGTDAPTPGNAENEPPEKETLSPPRRTPAPPEPGSPA |
| | | PGEGPSGRKRRRVPRDGRRAG |
| 1382 | CXXC1 | GGPNKIRQKCRLRQCQLRARESYKYFPSSLSPVTPSESLPR |
| | | PRRPLPTQQQPQPSQKLGRIR |
| 1383 | GORASP1 | PSYHKKPPGTPPPSALPLGAPPPDALPPGPTPEDSPSLETGS |
| | | RQSDYMEALLQAPGSSMEDP |
| 1384 | PRR14 | DPLESPPTAPDPALELPSTPPPSSLLRPRLSPWGLAPLFRSV |
| | | RSKLESFADIFLTPNKTPQP |
| 1385 | CRYZL2P-SEC16B | GTTTENTFYQDFSGCQGYSEAPGYRSALWLTPEQTCLLQP |
| | | SPQQPFPLQPGSYPAGGGAGQT |
| 1386 | NTRK1 | PFGQASASIMAAFMDNPFEFNPEDPIPVSFSPVDTNSTSGD |
| | | PVEKKDETPFGVSVAVGLAVF |
| 1387 | HMGXB3 | PGADVPTPSEGTSTSSPLPAPKKPTGADLLTPGSRAPELKG |
| | | RARGKPSLLAAARPMRAILPA |
| 1388 | HMX2 | KAPACFCPDQHGPKEQGPKHHPPIPFPCLGTPKGSGGSGPG |
| | | GLERTPFLSPSHSDFKEEKER |
| 1389 | MGA | KPLILSRKKDQATENTSPLNTPHTSANLVMTPQGQLLTLK |
| | | GPLFSGPVVAVSPDLLESDLKP |
| 1390 | FBF1 | LFPASPTREAHRESSVPVTPSVPPPASQHSTPAGLPPSRAKP |
| | | PTEGAGSPAKASQASKLRAS |
| 1391 | SULT1A1 | KCHRAPIFMRVPFLEFKAPGIPSGMETLKDTPAPRLLKTHL |
| | | PLALLPQTLLDQKVKVVYVAR |
| 1392 | KAT14 | SSSDRTPLTSPSPSPSLDFSAPGTPASHSATPSLLSEADLIPD |
| | | VMPPQALFHDDDEMEGDGV |
| 1393 | ELK1_0 | PERTPGSGSGSGLQAPGPALTPSLLPTHTLTPVLLTPSSLPP |
| | | SIHFWSTLSPIAPRSPAKLS |
| 1394 | ELK1_1 | GSGSGSGLQAPGPALTPSLLPTHTLTPVLLTPSSLPPSIHFW |
| | | STLSPIAPRSPAKLSFQFPS |
| 1395 | DAG1 | IHATPTPVTAIGPPTTAIQEPPSRIVPTPTSPAIAPPTETMAPP |
| | | VRDPVPGKPTVTIRTRGA |
| 1396 | GLIS1 | PLDATTSSHHHLSPLPMAESTRDGLGPGLLSPIVSPLKGLG |
| | | PPPLPPSSQSHSPGGQPFPTL |
| 1397 | PRDM2 | SSASPHPCPSPLSNATAQSPLPILSPTVSPSPSPIPPVEPLMSA |
| | | ASPGPPTLSSSSSSSSSS |
| 1398 | POU2F2_0 | WFCNRRQKEKRINPCSAAPMLPSPGKPASYSPHMVTPQG |
| | | GAGTLPLSQASSSLSTTVTTLSS |
| 1399 | POU2F2_1 | RQKEKRINPCSAAPMLPSPGKPASYSPHMVTPQGGAGTLP |
| | | LSQASSSLSTTVTTLSSAVGTL |
| 1400 | FOXN1_0 | KHAGFSCSSFVSDGPPERTPSLPPHSPRIASPGPEQVQGHCP |
| | | AGPGPGPFRLSPSDKYPGFG |
| 1401 | FOXN1_1 | APGPIPGKNPLQDLLMGHTPSCYGQTYLHLSPGLAPPGPP |
| | | QPLFPQPDGHLELRAQPGTPQD |
| 1402 | RIMS1 | DVELESESVSEKGDLDYYWLDPATWHSRETSPISSHPVTW |
| | | QPSKEGDRLIGRVILNKRTTMP |
| 1403 | MED12L | LYHTHPMPKPRSYYLQPLPLPPEEEEEEPTSPVSQEPERKS |
| | | AELSDQGKTTTDEEKKTKGRK |
| 1404 | REPIN1 | HKRSEGSAQAAPGPGSPQLPAGPQESAAEPTPAVPLKPAQ |
| | | EPPPGAPPEHPQDPIEAPPSLY |
| 1405 | WNK2_0 | SVPAPACPPSLQQHFPDPAMSFAPVLPPPSTPMPTGPGQPA |
| | | PPGQQPPPLAQPTPLPQVLAP |
| 1406 | WNK2_1 | TPLAGIDGLPPALPDLPTATVPPVPPPQYFSPAVILPSLAAP |
| | | LPPASPALPLQAVKLPHPPG |
| 1407 | WNK2_2 | VSASVQSVPTQTATLLPPANPPLPGGPGIASPCPTVQLTVE |
| | | PVQEEQASQDKPPGLPQSCES |
| 1408 | GTF3C2_0 | TPMPKKRGRKSKAELLLLKLSKDLDRPESQSPKRPPEDFET |
| | | PSGERPRRRAAQVALLYLQEL |
| 1409 | GTF3C2_1 | SKAELLLLKLSKDLDRPESQSPKRPPEDFETPSGERPRRRA |
| | | AQVALLYLQELAEELSTALPA |
| 1410 | BTBD18 | TQDSPQIPDPGGDFQEPSGTQPFSSNEQEMSPTRTELCQDS |
| | | PMCTKLQDILVSASHSPDHPV |
| 1411 | STXBP5 | TEVIPMLEVRLLYEINDVETPEGEQPPPLPTPVGGSNPQPIP |
| | | PQSHPSTSSSSSDGLRDNVP |
| 1412 | CNOT1 | CSNVMNKARQPPPGVMPKGRPPSASSLDAISPVQIDPLAG |
| | | MTSLSIGGSAAPHTQSMQGFPP |
| 1413 | CNOT4 | EGAVTESQSLFSDNFRHPNPIPSGLPPFPSSPQTSSDWPTAP |
| | | EPQSLFTSETIPVSSSTDWQ |
| 1414 | FETUB | SQAPATGSENSAVNQKPTNLPKVEESQQKNTPPTDSPSKA |
| | | GPRGSVQYLPDLDDKNSQEKGP |
| 1415 | BCL11A_0 | AMEPPAMDFSRRLRELAGNTSSPPLSPGRPSPMQRLLQPF |
| | | QPGSKPPFLATPPLPPLQSAPP |
| 1416 | BCL11A_1 | SSPPLSPGRPSPMQRLLQPFQPGSKPPFLATPPLPPLQSAPPP |
| | | SQPPVKSKSCEFCGKTFKF |
| 1417 | KDM3B | GPSLSAMGNGRSSSPTSSLTQPIEMPTLSSSPTEERPTVGPG |
| | | QQDNPLLKTFSNVFGRHSGG |
| 1418 | RBM10 | SQSYTIMSPAVLKSELQSPTHPSSALPPATSPTAQESYSQYP |
| | | VPDVSTYQYDETSGYYYDPQ |
| 1419 | KIF20A | KKRLGTNQENQQPNQQPPGKKPFLRNLLPRTPTCQSSTDC |
| | | SPYARILRSRRSPLLKSGPFGK |
| 1420 | DGKZ_0 | YVTEIAQDEIYILDPELLGASARPDLPTPTSPLPTSPCSPTPR |
| | | SLQGDAAPPQGEELIEAAK |
| 1421 | DGKZ_1 | AQDEIYILDPELLGASARPDLPTPTSPLPTSPCSPTPRSLQG |
| | | DAAPPQGEELIEAAKRNDFC |
| 1422 | DGKZ_2 | YILDPELLGASARPDLPTPTSPLPTSPCSPTPRSLQGDAAPP |
| | | QGEELIEAAKRNDFCKLQEL |
| 1423 | FOXF2 | PVPSSPAMASAIECHSPYTSPAAHWSSPGASPYLKQPPALT |
| | | PSSNPAASAGLHSSMSSYSLE |
| 1424 | HSPG2 | NKVGSAEAFAQLLVQGPPGSLPATSIPAGSTPTVQVTPQLE |
| | | TKSIGASVEFHCAVPSDRGTQ |
| 1425 | MIA3 | GSSPTRVLDEGKVNMAPKGPPPFPGVPLMSTPMGGPVPPP |
| | | IRYGPPPQLCGPFGPRPLPPPF |
| 1426 | CREB3L2_0 | PTPPSSHGSDSEGSLSPNPRLHPFSLPQTHSPSRAAPRAPSA |
| | | LSSSPLLTAPHKLQGSGPLV |
| 1427 | CREB3L2_1 | SPNPRLHPFSLPQTHSPSRAAPRAPSALSSSPLLTAPHKLQG |
| | | SGPLVLTEEEKRTLIAEGYP |
| 1428 | NFATC1_0 | PQRSTLMPAAPGVSPKLHDLSPAAYTKGVASPGHCHLGLP |
| | | QPAGEAPAVQDVPRPVATHPGS |
| 1429 | NFATC1_1 | PGHCHLGLPQPAGEAPAVQDVPRPVATHPGSPGQPPPALL |
| | | PQQVSAPPSSSCPPGLEHSLCP |
| 1430 | PDE5A | PVCKEGIRGHTESCSCPLQQSPRADNSAPGTPTRKISASEF |
| | | DRPLRPIVVKDSEGTVSFLSD |
| 1431 | PRDM15 | ELRVWYAAFYAKKMDKPMLKQAGSGVHAAGTPENSAPV |
| | | ESEPSQWACKVCSATFLELQLLNE |
| 1432 | MYBL2_0 | VTTPLHRDKTPLHQKHAAFVTPDQKYSMDNTPHTPTPFK |
| | | NALEKYGPLKPLPQTPHLEEDLK |
| 1433 | MYBL2_1 | HRDKTPLHQKHAAFVTPDQKYSMDNTPHTPTPFKNALEK |
| | | YGPLKPLPQTPHLEEDLKEVLRS |
| 1434 | ZYX | YVPPPVATPFSSKSSTKPAAGGTAPLPPWKSPSSSQPLPQV |
| | | PAPAQSQTQFHVQPQPQPKPQ |
| 1435 | FCMR | ARGADAAGTGEAPVPGPGAPLPPAPLQVSESPWLHAPSLK |
| | | TSCEYVSLYHQPAAMMEDSDSD |
| 1436 | ATG12_0 | MAEEPQSVLQLPTSIAAGGEGLTDVSPETTTPEPPSSAAVS |
| | | PGTEEPAGDTKKKIDILLKAV |
| 1437 | ATG12_1 | LPTSIAAGGEGLTDVSPETTTPEPPSSAAVSPGTEEPAGDTK |
| | | KKIDILLKAVGDTPIMKTKK |
| 1438 | DLGAP2 | LCSGHTCGLAPPEDCEHLHHGPDARPPYLLSPADSCPGGR |
| | | HRCSPRSSVHSECVMMPVVLGD |
| 1439 | DNM3_0 | LGIIGDISTATVSTPAPPPVDDSWIQHSRRSPPPSPTTQRRPT |
| | | LSAPLARPTSGRGPAPAIP |
| 1440 | DNM3_1 | PPSPTTQRRPTLSAPLARPTSGRGPAPAIPSPGPHSGAPPVP |
| | | FRPGPLPPFPSSSDSFGAPP |
| 1441 | KLF16 | LAASILADLRGGPGAAPGGASPASSSSAASSPSSGRAPGAA |
| | | PSAAAKSHRCPFPDCAKAYYK |
| 1442 | WNT6 | TQACSMGELLQCGCQAPRGRAPPRPSGLPGTPGPPGPAGS |
| | | PEGSAAWEWGGCGDDVDFGDEK |
| 1443 | MUC16_0 | MTYTEKSEVSSSIHPRPETSAPGAETTLTSTPGNRAISLTLP |
| | | FSSIPVEEVISTGITSGPDI |
| 1444 | MUC16_1 | RGPGDMSWQSSPSLENPSSLPSLLSLPATTSPPPISSTLPVTI |
| | | SSSPLPVTSLLTSSPVTTT |
| 1445 | MUC16_2 | PEDVSWPSPLSVEKNSPPSSLVSSSSVTSPSPLYSTPSGSSHS |
| | | SPVPVTSLFTSIMMKATDM |
| 1446 | BCAR1 | ETYDVPPAFAKAKPFDPARTPLVLAAPPPDSPPAEDVYDV |
| | | PPPAPDLYDVPPGLRRPGPGTL |
| 1447 | FOXO4 | APGPSSLVPTLSMIAPPPVMASAPIPKALGTPVLTPPTEAAS |
| | | QDRMPQDLDLDMYMENLECD |
| 1448 | AKT1S1 | RCLHDIALAHRAATAARPPAPPPAPQPPSPTPSPPRPTLARE |
| | | DNEEDEDEPTETETSGEQLG |
| 1449 | COL5A2 | AIGTDGTPGAKGPTGSPGTSGPPGSAGPPGSPGPQGSTGPQ |
| | | GIRGQPGDPGVPGFKGEAGPK |
| 1450 | CTC1_0 | SYLPPARWNSSGEGHLELWDAPVPVFPLTISPGPVTPIPVL |
| | | YPESASCLLRLRNKLRGVQRN |
| 1451 | CTC1_1 | ARWNSSGEGHLELWDAPVPVFPLTISPGPVTPIPVLYPESA |
| | | SCLLRLRNKLRGVQRNLAGSL |
| 1452 | SH2D6 | PLSLAPAHLPGTEEDSLYLDHSGPLGPSKPSPPLPQPTMLK |
| | | GAVSLPVAGKQGPIFGRREQG |
| 1453 | KSR1 | DSSSNPSSTTSSTPSSPAPFPTSSNPSSATTPPNPSPGQRDSR |
| | | FNFPAAYFIHHRQQFIFPV |
| 1454 | C1orf127_0 | AAPVLWTVESFFQCVGSGTESPASTAALRTTPSPPSPGPET |
| | | PPAGVPPAASSQVWAAGPAAQ |
| 1455 | C1orf127_1 | WTVESFFQCVGSGTESPASTAALRTTPSPPSPGPETPPAGV |
| | | PPAASSQVWAAGPAAQEWLSR |
| 1456 | C1orf127_2 | FFQCVGSGTESPASTAALRTTPSPPSPGPETPPAGVPPAASS |
| | | QVWAAGPAAQEWLSRDLLHR |
| 1457 | C1orf127_3 | QTSASILPRVVQAQRGPQPPPGEAGIPGHPTPPATLPSEPVE |
| | | GVQASPWRPRPVLPTHPALT |
| 1458 | C1orf127_4 | GVQASPWRPRPVLPTHPALTLPVSSDASSPSPPAPRPERPES |
| | | LLVSGPSVTLTEGLGTVRPE |
| 1459 | C1orf127_5 | GHMDLSSSEPSQDIEGPGLSILPARDATFSTPSVRQPDPSA |
| | | WLSSGPELTGMPRVRLAAPLA |
| 1460 | C2CD4D_0 | AEPAARWAPSGLFSKRRAPGPPTSACPNVLTPDRIPQFFIPP |
| | | RLPDPGGAVPAARRHVAGRG |
| 1461 | C2CD4D_1 | SDTASSPDSSPFGSPRPGLGRRRVSRPHSLSPEKASSADTSP |
| | | HSPRRAGPPTPPLFHLDFLC |
| 1462 | LHX6 | TLQKLADMTGLSRRVIQVWFQNCRARHKKHTPQHPVPPS |
| | | GAPPSRLPSALSDDIHYTPFSSP |
| 1463 | FRMD1 | MAVPPRGRGIDPARTNPDTFPPSGARCMEPSPERPACSQQ |
| | | EPTLGMDAMASEHRDVLVLLPS |
| 1464 | SPHK2_0 | TLGTVLGLATLHTYRGRLSYLPATVEPASPTPAHSLPRAKS |
| | | ELTLTPDPAPPMAHSPLHRSV |
| 1465 | SPHK2_1 | GRLSYLPATVEPASPTPAHSLPRAKSELTLTPDPAPPMAHS |
| | | PLHRSVSDLPLPLPQPALASP |
| 1466 | SPHK2_2 | EPASPTPAHSLPRAKSELTLTPDPAPPMAHSPLHRSVSDLP |
| | | LPLPQPALASPGSPEPLPILS |
| 1467 | SPHK2_3 | AGDWGGAGDAPLSPDPLLSSPPGSPKAALHSPVSEGAPVIP |
| | | PSSGLPLPTPDARVGASTCGP |
| 1468 | LACTB | GAAPAQSPAAPDPEASPLAEPPQEQSLAPWSPQTPAPPCSR |
| | | CFARAIESSRDLLHRIKDEVG |
| 1469 | SMAD2 | YISEDGETSDQQLNQSMDTGSPAELSPTTLSPVNHSLDLQP |
| | | VTYSEPAFWCSIAYYELNQRV |
| 1470 | TET3_0 | AKEKNISLQTAIAIEALTQLSSALPQPSHSTPQASCPLPEAL |
| | | SPPAPFRSPQSYLRAPSWPV |
| 1471 | TET3_1 | KRSLFLEQVHDTSFPAPSEPSAPGWWPPPSSPVPRLPDRPP |
| | | KEKKKKLPTPAGGPVGTEKAA |
| 1472 | COL1A1 | PPGERGGPGSRGFPGADGVAGPKGPAGERGSPGPAGPKGS |
| | | PGEAGRPGEAGLPGAKGLTGSP |
| 1473 | PER1_0 | RDFTQEKSVFCRIRGGPDRDPGPRYQPFRLTPYVTKIRVSD |
| | | GAPAQPCCLLIAERIHSGYEA |
| 1474 | PER1_1 | HQNPRAEAPCYVSHPSPVPPSTPWPTPPATTPFPAVVQPYP |
| | | LPVFSPRGGPQPLPPAPTSVP |
| 1475 | PER1_2 | LPNYLFPTPSSYPYGALQTPAEGPPTPASHSPSPSLPALAPS |
| | | PPHRPDSPLFNSRCSSPLQL |
| 1476 | CARMIL1 | ENRFGLGTPEKNTKAEPKAEAGSRSRSSSSTPTSPKPLLQS |
| | | PKPSLAARPVIPQKPRTASRP |
| 1477 | CDCA8 | VGRLEVSMVKPTPGLTPRFDSRVFKTPGLRTPAAGERIYNI |
| | | SGNGSPLADSKEIFLTVPVGG |
| 1478 | AMPH_0 | AFTIQGAPSDSGPLRIAKTPSPPEEPSPLPSPTASPNHTLAPA |
| | | SPAPARPRSPSQTRKGPPV |
| 1479 | AMPH_1 | LRIAKTPSPPEEPSPLPSPTASPNHTLAPASPAPARPRSPSQT |
| | | RKGPPVPPLPKVTPTKELQ |
| 1480 | POGZ_0 | QKKGKSLDSEPSVPSAAKPPSPEKTAPVASTPSSTPIPALSP |
| | | PTKVPEPNENVGDAVQTKLI |
| 1481 | POGZ_1 | PSVPSAAKPPSPEKTAPVASTPSSTPIPALSPPTKVPEPNEN |
| | | VGDAVQTKLIMLVDDFYYGR |
| 1482 | POGZ_2 | AGATPAEPEELLTPLAPALPSPASTATPPPTPTHPQALALPP |
| | | LATEGAECLNVDDQDEGSPV |
| 1483 | NRIP1 | YARTSVIESPSTNRTTPVSTPPLLTSSKAGSPINLSQHSLVIK |
| | | WNSPPYVCSTQSEKLTNTA |
| 1484 | CHRNA4 | ATSGTQSLHPPSPSFCVPLDVPAEPGPSCKSPSDQLPPQQPL |
| | | EAEKASPHPSPGPCRPPHGT |
| 1485 | PIK3R2 | RPRGPRPLPARPRDGAPEPGLTLPDLPEQFSPPDVAPPLLV |
| | | KLVEAIERTGLDSESHYRPEL |
| 1486 | ADAM17 | LSLFHPSNVEMLSSMDSASVRIIKPFPAPQTPGRLQPAPVIP |
| | | SAPAAPKLDHQRMDTIQEDP |
| 1487 | PXN_0 | LLLELNAVQHNPPGFPADEANSSPPLPGALSPLYGVPETNS |
| | | PLGGKAGPLTKEKPKRNGGRG |
| 1488 | PXN_1 | NPPGFPADEANSSPPLPGALSPLYGVPETNSPLGGKAGPLT |
| | | KEKPKRNGGRGLEDVRPSVES |
| 1489 | SNAI2 | THTVIISPYLYESYSMPVIPQPEILSSGAYSPITVWTTAAPFH |
| | | AQLPNGLSPLSGYSSSLGR |
| 1490 | IRS2 | NSASVENVSLRKSSEGGVGVGPGGGDEPPTSPRQLQPAPP |
| | | LAPQGRPWTPGQPGGLVGCPGS |
| 1491 | USP10_0 | DGTGSASGTLPVSQPKSWASLFHDSKPSSSSPVAYVETKY |
| | | SPPAISPLVSEKQVEVKEGLVP |
| 1492 | USP10_1 | PVSQPKSWASLFHDSKPSSSSPVAYVETKYSPPAISPLVSE |
| | | KQVEVKEGLVPVSEDPVAIKI |
| 1493 | USP10_2 | KSWASLFHDSKPSSSSPVAYVETKYSPPAISPLVSEKQVEV |
| | | KEGLVPVSEDPVAIKIAELLE |
| 1494 | GFI1B | EPELEQDQNLARMAPAPEGPIVLSRPQDGDSPLSDSPPFYK |
| | | PSFSWDTLATTYGHSYRQAPS |
| 1495 | LPA | PVTESSVLTTPTVAPVPSTEAPSEQAPPEKSPVVQDCYHGD |
| | | GRSYRGISSTTVTGRTCQSWS |
| 1496 | TNKS1BP1 | QTPEASQASPCPAVTPSAPSAALPDEGSRHTPSPGLPAEGA |
| | | PEAPRPSSPPPEVLEPHSLDQ |
| 1497 | NIPBL_0 | YQQTTISHSPSSRFVPPQTSSGNRFMPQQNSPVPSPYAPQSP |
| | | AGYMPYSHPSSYTTHPQMQQ |
| 1498 | NIPBL_1 | SSRFVPPQTSSGNRFMPQQNSPVPSPYAPQSPAGYMPYSHP |
| | | SSYTTHPQMQQASVSSPIVAG |
| 1499 | FOXL2 | AHHLHAAAAPPPAPPHHGAAAPPPGQLSPASPATAAPPAP |
| | | APTSAPGLQFACARQPELAMMH |
| 1500 | PLEKHG5 | AGTHGTPSAPSRSLSELCLAVPAPGIRTQGSPQEAGPSWDC |
| | | RGAPSPGSGPGLVGCLAGEPA |
| 1501 | COL11A1 | DDGMRGEDGEIGPRGLPGEAGPRGLLGPRGTPGAPGQPG |
| | | MAGVDGPPGPKGNMGPQGEPGPP |
| 1502 | PSD4 | SQDRDEREGGHPQESLPCTLAPCPWRSPASSPEPSSPESESR |
| | | GPGPRPSPASSQEGSPQLQH |
| 1503 | MAP4K1_0 | ESSDDDYDDVDIPTPAEDTPPPLPPKPKFRSPSDEGPGSMG |
| | | DDGQLSPGVLVRCASGPPPNS |
| 1504 | MAP4K1_1 | PSDEGPGSMGDDGQLSPGVLVRCASGPPPNSPRPGPPPSTS |
| | | SPHLTAHSEPSLWNPPSRELD |
| 1505 | COL3A1_0 | GPGAAGFPGARGLPGPPGSNGNPGPPGPSGSPGKDGPPGP |
| | | AGNTGAPGSPGVSGPKGDAGQP |
| 1506 | COL3A1_1 | AAGIKGHRGFPGNPGAPGSPGPAGQQGAIGSPGPAGPRGP |
| | | VGPSGPPGKDGTSGHPGPIGPP |
| 1507 | TBKBP1_0 | SSLQGRILRTLLQEQARSGGQRHSPLSQRHSPAPQCPSPSPP |
| | | ARAAPPCPPCQSPVPQRRSP |
| 1508 | TBKBP1_1 | SPAPQCPSPSPPARAAPPCPPCQSPVPQRRSPVPPCPSPQQR |
| | | RSPASPSCPSPVPQRRSPVP |
| 1509 | TBKBP1_2 | RAAPPCPPCQSPVPQRRSPVPPCPSPQQRRSPASPSCPSPVP |
| | | QRRSPVPPSCQSPSPQRRSP |
| 1510 | TBKBP1_3 | CQSPVPQRRSPVPPCPSPQQRRSPASPSCPSPVPQRRSPVPP |
| | | SCQSPSPQRRSPVPPSCPAP |
| 1511 | INSYN1 | LDVSTPSDSVDGPESTRPGAGPDYRLMNGGTPIPNGPRVE |
| | | TPDSSSEEAFGAGPTVKSQLPQ |
| 1512 | PLEKHA4 | HRMMTGGNLDSQGDPLPGVPLPPSDPTRQETPPPRSPPVA |
| | | NSGSTGFSRRGSGRGGGPTPWG |
| 1513 | GIGYF2 | LSQIPSDTASPLLILPPPVPNPSPTLRPVETPVVGAPGMGSV |
| | | STEPDDEEGLKHLEQQAEKM |
| 1514 | YIF1B | AVDTMYVGRKLGLLFFPYLHQDWEVQYQQDTPVAPRFD |
| | | VNAPDLYIPAMAFITYVLVAGLAL |
| 1515 | EIF4ENIF1 | SQANRYTKEQDYRPKATGRKTPTLASPVPTTPFLRPVHQV |
| | | PLVPHVPMVRPAHQLHPGLVQR |
| 1516 | KAT5 | IPGGEPDQPLSSSSCLQPNHRSTKRKVEVVSPATPVPSETAP |
| | | ASVFPQNGAARRAVAAQPGR |
| 1517 | MICALL1_0 | IMTYVSQYYNHFCSPGQAGVSPPRKGLAPCSPPSVAPTPV |
| | | EPEDVAQGEELSSGSLSEQGTG |
| 1518 | MICALL1_1 | PFEEEEEDKEEEAPAAPSLATSPALGHPESTPKSLHPWYGI |
| | | TPTSSPKTKKRPAPRAPSASP |
| 1519 | MICALL1_2 | EAPAAPSLATSPALGHPESTPKSLHPWYGITPTSSPKTKKR |
| | | PAPRAPSASPLALHASRLSHS |
| 1520 | MICALL1_3 | APSLATSPALGHPESTPKSLHPWYGITPTSSPKTKKRPAPR |
| | | APSASPLALHASRLSHSEPPS |
| 1521 | MED26 | HTSSPGLGKPPGPCLQPKASVLQQLDRVDETPGPPHPKGPP |
| | | RCSFSPRNSRHEGSFARQQSL |
| 1522 | ANKRD40_0 | VPNYLANPAFPFIYTPTAEDSAQMQNGGPSTPPASPPADGS |
| | | PPLLPPGEPPLLGTFPRDHTS |
| 1523 | ANKRD40_1 | PFIYTPTAEDSAQMQNGGPSTPPASPPADGSPPLLPPGEPPL |
| | | LGTFPRDHTSLALVQNGDVS |
| 1524 | DBP | AALPAATTPGPGLETAGPADAPAGAVVGGGSPRGRPGPV |
| | | PAPGLLAPLLWERTLPFGDVEYV |
| 1525 | FHIP1B_0 | ALFLRQQSLGGSESPGPAPCSPGLSASPASSPGRRPTPAEEP |
| | | GELEDNYLEYLREARRGVDR |
| 1526 | FHIP1B_1 | SPLEPPLPLEEEEAYESFTCPPEPPGPFLSSPLRTLNQLPSQP |
| | | FTGPFMAVLFAKLENMLQN |
| 1527 | EXOSC10 | ALADFIHQQRTQQVEQDMFAHPYQYELNHFTPADAVLQK |
| | | PQPQLYRPIEETPCHFISSLDEL |
| 1528 | KRTAP10-7 | CSDSWQVDDCPESCCEPPCCAPAPCLSLVCTPVSYVSSPCC |
| | | RVTCEPSPCQSGCTSSCTPSC |
| 1529 | KIAA0754_0 | EEPTSPAAAVPTPEEPASPAAAVPTPEEPASPAAAVPTPEEP |
| | | AFPAPAVPTPEESASAAVAV |
| 1530 | KIAA0754_1 | AAVPTPEEPASPAAAVPTPEEPAFPAPAVPTPEESASAAVA |
| | | VPTPEESASPAAAVPTPAESA |
| 1531 | KIAA0754_2 | AVVATLEEPTSPAASVPTPAAMVATLEEFTSPAASVPTSEE |
| | | PASLAAAVSNPEEPTSPAAAV |
| 1532 | ATG9B_0 | FSPPTAGPPCSVLQGTGASQSCHSALPIPATPPTQAQPAMT |
| | | PASASPSWGSHSTPPLAPATP |
| 1533 | ATG9B_1 | SVLQGTGASQSCHSALPIPATPPTQAQPAMTPASASPSWGS |
| | | HSTPPLAPATPTPSQQCPQDS |
| 1534 | ATG9B_2 | TGASQSCHSALPIPATPPTQAQPAMTPASASPSWGSHSTPP |
| | | LAPATPTPSQQCPQDSPGLRV |
| 1535 | ATG9B_3 | PTQAQPAMTPASASPSWGSHSTPPLAPATPTPSQQCPQDSP |
| | | GLRVGPLIPEQDYERLEDCDP |
| 1536 | ILF3 | RDSSKGEDSAEETEAKPAVVAPAPVVEAVSTPSAAFPSDA |
| | | TAEQGPILTKHGKNPVMELNEK |
| 1537 | SLC25A46 | RSFSTGSDLGHWVTTPPDIPGSRNLHWGEKSPPYGVPTTST |
| | | PYEGPTEEPFSSGGGGSVQGQ |
| 1538 | CBS | PEDKEAKEPLWIRPDAPSRCTWQLGRPASESPHHHTAPAK |
| | | SPKILPDILKKIGDTPMVRINK |
| 1539 | PELP1 | SSFCSEALVTCAALTHPRVPPLQPMGPTCPTPAPVPPPEAP |
| | | SPFRAPPFHPPGPMPSVGSMP |
| 1540 | PAK5 | QKFTGLPQQWHSLLADTANRPKPMVDPSCITPIQLAPMKT |
| | | IVRGNKPCKETSINGLLEDFDN |
| 1541 | NR4A3_0 | DPPMKAVPTVAGARFPLFHFKPSPPHPPAPSPAGGHHLGY |
| | | DPTAAAALSLPLGAAAAAGSQA |
| 1542 | NR4A3_1 | GSQAAALESHPYGLPLAKRAAPLAFPPLGLTPSPTASSLLG |
| | | ESPSLPSPPSRSSSSGEGTCA |
| 1543 | TFAP2A | HDGTSNGTARLPQLGTVGQSPYTSAPPLSHTPNADFQPPY |
| | | FPPPYQPIYPQSQDPYSHVNDP |
| 1544 | FAM161A | IKREKILADIEADEENLKETRWPYLSPRRKSPVRCAGVNPV |
| | | PCNCNPPVPTVSSRGREQAVR |
| 1545 | ADAMTS14 | HRLCCVSCIKKASGPNPGPDPGPTSLPPFSTPGSPLPGPQDP |
| | | ADAAEPPGKPTGSEDHQHGR |
| 1546 | FNDC3A | VQVNPGEAFTIRREDGQFQCITGPAQVPMMSPNGSVPPIY |
| | | VPPGYAPQVIEDNGVRRVVVVP |
| 1547 | GDF6 | GAELRLFRQAPSAPWGPPAGPLHVQLFPCLSPLLLDARTL |
| | | DPQGAPPAGWEVFDVWQGLRHQ |
| 1548 | ZMYND8 | SASEESMDFLDKSTASPASTKTGQAGSLSGSPKPFSPQLSA |
| | | PITTKTDKTSTTGSILNLNLD |
| 1549 | SOX8 | QGDYGDLQASSYYGAYPGYAPGLYQYPCFHSPRRPYASP |
| | | LLNGLALPPAHSPTSHWDQPVYT |
| 1550 | ROBO4 | QTQPPVAPQAPSSILLPAAPIPILSPCSPPSPQASSLSGPSPAS |
| | | SRLSSSSLSSLGEDQDSV |
| 1551 | MYO15A_0 | SPPVPPRPPSSGPPPAPPLSPALSGLPRPASPYGSLRRHPPP |
| | | WAAPAHVPPAPQASWWAFVE |
| 1552 | MYO15A_1 | RRHPPPWAAPAHVPPAPQASWWAFVEPPAVSPEVPPDLL |
| | | AFPGPRPSFRGSRRRGAAFGFPG |
| 1553 | MYO15A_2 | PPFLPPARRPRSLQESPAPRRAAGRLGPPGSPLPGSPRPPSP |
| | | PLGLCHSPRRSSLNLPSRLP |
| 1554 | MYO15A_3 | SLPAEKPPAPEAQPTSVGTGPPAKPVLLRATPKPLAPAPLA |
| | | KAPRLPIKPVAAPVLAQDQAS |
| 1555 | NCOR2_0 | PKPPATLGADGPPPGPPTPPPEDIPAPTEPTPASEATGAPTPP |
| | | PAPPSPSAPPPVVPKEEKE |
| 1556 | NCOR2_1 | GPPPGPPTPPPEDIPAPTEPTPASEATGAPTPPPAPPSPSAPPP |
| | | VVPKEEKEEETAAAPPVE |
| 1557 | ELK3 | AAAASAFLASSVSAKISSLMLPNAASISSASPFSSRSPSLSP |
| | | NSPLPSEHRSLFLEAACHDS |
| 1558 | E2F7_0 | VGPSSGQLPSFSVPCMVLPSPPLGPFPVLYSPAMPGPVSST |
| | | LGALPNTGPVNFSLPGLGSIA |
| 1559 | E2F7_1 | SHSVVQQPESPVYVGHPVSVVKLHQSPVPVTPKSIQRTHR |
| | | ETFFKTPGSLGDPVLKRRERNQ |
| 1560 | KLF4 | GLMGKFVLKASLSAPGSEYGSPSVISVSKGSPDGSHPVVV |
| | | APYNGGPPRTCPKIKQEAVSSC |
| 1561 | PKD1 | WEPLKVLLEALYFSLVAKRLHPDEDDTLVESPAVTPVSAR |
| | | VPRVRPPHGFALFLAKEEARKV |
| 1562 | ATXN2_0 | VPWPSPCPSPSSRPPSRYQSGPNSLPPRAATPTRPPSRPPSRP |
| | | SRPPSHPSAHGSPAPVSTM |
| 1563 | ATXN2_1 | NPNAKEFNPRSFSQPKPSTTPTSPRPQAQPSPSMVGHQQPT |
| | | PVYTQPVCFAPNMMYPVPVSP |
| 1564 | ATXN2_2 | SFSQPKPSTTPTSPRPQAQPSPSMVGHQQPTPVYTQPVCFA |
| | | PNMMYPVPVSPGVQPLYPIPM |
| 1565 | ATXN2_3 | SPSMVGHQQPTPVYTQPVCFAPNMMYPVPVSPGVQPLYPI |
| | | PMTPMPVNQAKTYRAVPNMPQQ |
| 1566 | KNG1 | IQSDDDWIPDIQIDPNGLSFNPISDFPDTTSPKCPGRPWKSV |
| | | SEINPTTQMKESYYFDLTDG |
| 1567 | ULK1 | SHGLQSCRNLRGSPKLPDFLQRNPLPPILGSPTKAVPSFDFP |
| | | KTPSSQNLLALLARQGVVMT |
| 1568 | WEE1_0 | EEEEEEEGSGHSTGEDSAFQEPDSPLPPARSPTEPGPERRRS |
| | | PGPAPGSPGELEEDLLLPGA |
| 1569 | WEE1_1 | FQEPDSPLPPARSPTEPGPERRRSPGPAPGSPGELEEDLLLP |
| | | GACPGADEAGGGAEGDSWEE |
| 1570 | COL2A1_0 | PAGEQGPRGDRGDKGEKGAPGPRGRDGEPGTPGNPGPPG |
| | | PPGPPGPPGLGGNFAAQMAGGFD |
| 1571 | COL2A1_1 | LVGPRGERGFPGERGSPGAQGLQGPRGLPGTPGTDGPKGA |
| | | SGPAGPPGAQGPPGLQGMPGER |
| 1572 | COL2A1_2 | APGASGDRGPPGPVGPPGLTGPAGEPGREGSPGADGPPGR |
| | | DGAAGVKGDRGETGAVGAPGAP |
| 1573 | CACNA1G | LQLPKDAPHLLQPHSAPTWGTIPKLPPPGRSPLAQRPLRRQ |
| | | AAIRTDSLDVQGLGSREDLLA |
| 1574 | PTK2_0 | RMESRRQATVSWDSGGSDEAPPKPSRPGYPSPRSSEGFYP |
| | | SPQHMVQTNHYQVSGYPGSHGI |
| 1575 | PTK2_1 | SWDSGGSDEAPPKPSRPGYPSPRSSEGFYPSPQHMVQTNH |
| | | YQVSGYPGSHGITAMAGSIYPG |
| 1576 | TAB3 | QSSPQGPVPHYSQRPLPVYPHQQNYQPSQYSPKQQQIPQS |
| | | AYHSPPPSQCPSPFSSPQHQVQ |
| 1577 | FCRLA | ETASVVAITVQELFPAPILRAVPSAEPQAGSPMTLSCQTKL |
| | | PLQRSAARLLFSFYKDGRIVQ |
| 1578 | PTCH1 | LNGLVLLPVLLSFFGPYPEVSPANGLNRLPTPSPEPPPSVVR |
| | | FAMPPGHTHSGSDSSDSEYS |
| 1579 | ZNF804A | CEVYQHILQPNMLANKVKFTFPPAALPPPSTPLQPLPLQQS |
| | | LCSTSVTTIHHTVLQQHAAAA |
| 1580 | RGS12 | VQESSDSPSTSPGSASSPPGPPGTTPPGQKSPSGPFCTPQSP |
| | | VSLAQEGTAQIWKRQSQEVE |
| 1581 | COL5A1_0 | FPGDRGLPGPVGALGLKGNEGPPGPPGPAGSPGERGPAGA |
| | | AGPIGIPGRPGPQGPPGPAGEK |
| 1582 | COL5A1_1 | ERGEKGESGPSGAAGPPGPKGPPGDDGPKGSPGPVGFPGD |
| | | PGPPGEPGPAGQDGPPGDKGDD |
| 1583 | COL5A1_2 | PIGPQGAPGKPGPDGLRGIPGPVGEQGLPGSPGPDGPPGPM |
| | | GPPGLPGLKGDSGPKGEKGHP |
| 1584 | PAK4 | APNGPSAGGLAIPQSSSSSSRPPTRARGAPSPGVLGPHASEP |
| | | QLAPPACTPAAPAVPGPPGP |
| 1585 | SFPQ | GVGSAPPASSSAPPATPPTSGAPPGSGPGPTPTPPPAVTSAP |
| | | PGAPPPTPPSSGVPTTPPQA |
| 1586 | ANKRD11 | DSPMPPSMEDRAPLPPVPAEKFACLSPGYYSPDYGLPSPK |
| | | VDALHCPPAAVVTVTPSPEGVF |
| 1587 | TICRR_0 | TPRTPKRQGTQPPGFLPNCTWPHSVNSSPESPSCPAPPTSST |
| | | AQPRRECLTPIRDPLRTPPR |
| 1588 | TICRR_1 | PALSMPRASRSLSKPEPTYVSPPCPRLSHSTPGKSRGQTYIC |
| | | QACTPTHGPSSTPSPFQTDG |
| 1589 | PSMB8 | APRGQRPESALPVAGSGRRSDPGHYSFSMRSPELALPRGM |
| | | QPTEFFQSLGGDGERNVQIEMA |
| 1590 | STIM1_0 | LAKKALLALNHGLDKAHSLMELSPSAPPGGSPHLDSSRSH |
| | | SPSSPDPDTPSPVGDSRALQAS |
| 1591 | STIM1_1 | HGLDKAHSLMELSPSAPPGGSPHLDSSRSHSPSSPDPDTPS |
| | | PVGDSRALQASRNTRIPHLAG |
| 1592 | CAPN15 | MLEPGEYAVVCCAFNHWGPPLPGTPAPQASSPSAGVPRAS |
| | | PEPPGHVLAVYSSRLVMVEPVE |
| 1593 | BAHCC1 | PTAPGAPSPAAGPTKLPPCCHPPDPKPPASSPTPPPRPSAPC |
| | | TLNVCPASSPGPGSRVRSAE |
| 1594 | KIAA1210_0 | QVIIRGLPVWFSHFQGILEGSLQCVTQTLETPNLDEPLPVEP |
| | | KEEEPNLPLVSEEEKSITKP |
| 1595 | KIAA1210_1 | GNLTKISYVADKQQSRPKSESMAKKQPACKTPGKPAGQQ |
| | | SDYAVSEPVWITMAKQKQKSFKA |
| 1596 | MYO9B_0 | ASTESLLEERAGRGASEGPPAPALPCPGAPTPSPLPTVAAP |
| | | PRRRPSSFVTVRVKTPRRTPI |
| 1597 | MYO9B_1 | WAPGAREAAAPVRRREPPARRPDQIHSVYITPGADLPVQG |
| | | ALEPLEEDGQPPGAKRRYSDPP |
| 1598 | TRPM2 | FRGAVYHSYLTIFGQIPGYIDGVNFNPEHCSPNGTDPYKPK |
| | | CPESDATQQRPAFPEWLTVLL |
| 1599 | TBX10 | AFLSAGLGILAPSETYPLPTTSSGWEPRLGSPFPSGPCTSST |
| | | GAQAVAEPTGQGPKNPRVSR |
| 1600 | C11orf53 | ALLEPYFPQEPYGDYRPPALTPNAGSLFSASPLPPLLPPPFP |
| | | GDPAHFLFRDSWEQTLPDGL |
| 1601 | UNC13A | LPPAAPGKEDKAPVAPTEAPDMAKVAPKPATPDKVPAAE |
| | | QIPEAEPPKDEESFRPREDEEGQ |
| 1602 | AGAP2_0 | VPPGPPLSGGLSPDPKPGGAPTSSRRPLLSSPSWGGPEPEG |
| | | RAGGGIPGSSSPHPGTGSRRL |
| 1603 | AGAP2_1 | KGKSKTLDNSDLHPGPPAGSPPPLTLPPTPSPATAVTAASA |
| | | QPPGPAPPITLEPPAPGLKRG |
| 1604 | SOCS1 | VAHNQVAADNAVSTAAEPRRRPEPSSSSSSSPAAPARPRP |
| | | CPAVPAPAPGDTHFRTFRSHAD |
| 1605 | SPATA31D4 | LADLFSPSPLRDPLPPQPVSPLDSKFPIDHSPPQQLPFPLLPP |
| | | HHIERVEPSLQPEASLSLN |
| 1606 | KIAA1671 | IIDVDALWSHRGSEDGPRPQSNWKESANKMSPSGGAPQTT |
| | | PTLRSRPKDLPVRRKTDVISDT |
| 1607 | PROX2 | RVQLQAGVPVGNLSLAKRLDSPRYPIPPRMTPKPCQDPPA |
| | | NFPLTAPSHIQENQILSQLLGH |
| 1608 | LRRC37A3 | PEHSHLTQATVQPLDLGFTITPESMTEVELSPTMKETPTQP |
| | | PKKVVPQLRVYQGVTNPTPGQ |
| 1609 | POM121L2 | TIWSLRHPRPIWSPVTIRITPPDQRVPPSTSPEDVIALAGLPP |
| | | SEELADPCSKETVLRALRE |
| 1610 | LRRC66 | SAHYSEVPYGDPRDTGPSVFPPRWDSGLDVTPANKEPVQ |
| | | KSTPSDTCCELESDCDSDEGSLF |
| 1611 | KIF26A_0 | LQAPASHEDLDAPHGGPSLAPPSTTTSSRDTPGPAGPAGR |
| | | QPGRAGPDRTKGLAWSPGPSVQ |
| 1612 | KIF26A_1 | TSSRDTPGPAGPAGRQPGRAGPDRTKGLAWSPGPSVQVS |
| | | VAPAGLGGALSTVTIQAQQCLEG |
| 1613 | KIF26A_2 | TFAELQERLECMDGNEGPSGGPGGTDGAQASPARGGRKP |
| | | SPPEAASPRKAVGTPMAASTPRG |
| 1614 | KIF26A_3 | LAPKAGFLPRPSGAAPPAPPTRKSSLEQRSSPASAPPHAVN |
| | | PARVGAAAVLRGEEEPRPSSR |
| 1615 | PRRC2B | DQKCKQARKAGEARKQAEKEVPWSPSAEKASPQENGPA |
| | | VHKGSPEFPAQETPTTFPEEAPTV |
| 1616 | BNC1 | KGQPAFPNIGQNGVLFPNLKTVQPVLPFYRSPATPAEVAN |
| | | TPGILPSLPLLSSSIPEQLISN |
| 1617 | SPATA31D3 | LADLFSPSPLRDPLPPQPVSPLDSKFPIDHSPPQQLPFPLLPP |
| | | HHIERVEPSLQPEASLSLN |
| 1618 | CXorf49_0 | ADTSRQASFHCKESYLPVPGRFLTSAPRGLTPVAERPAVG |
| | | ELEDSPQKKMQSRAWGKVEVRP |
| 1619 | CXorf49_1 | RLSVRRGEFSSSDPNIRAPQLPGTSEPSAYSPGGLVPRRHA |
| | | PSGNQQPPVHPPRPERQQQPP |
| 1620 | CXorf49B_0 | ADTSRQASFHCKESYLPVPGRFLTSAPRGLTPVAERPAVG |
| | | ELEDSPQKKMQSRAWGKVEVRP |
| 1621 | CXorf49B_1 | RLSVRRGEFSSSDPNIRAPQLPGTSEPSAYSPGGLVPRRHA |
| | | PSGNQQPPVHPPRPERQQQPP |
| 1622 | TNRC18_0 | ALKAKVIQKLEDVSKPPAYAYPATPSSHPTSPPPASPPPTP |
| | | GITRKEEAPENVVEKKDLELE |
| 1623 | TNRC18_1 | AATLEEGNPTDEVPSTPLALEPSSTPGSKKSPPEPVDKRAK |
| | | APKARPAPPQPSPAPPAFTSC |
| 1624 | RNF225 | RPQLVALAPAPGFSWFPPRPPPGSPWAPAWTPRPTGPDLD |
| | | TALPGTAEDALEPEAGPEDPAE |
| 1625 | PCNX3_0 | GLLSSEGPSGKWSLGGRKGLGGSDGEPASGSPKGGTPKSQ |
| | | APLDLSLSLSLSLSPDVSTEAS |
| 1626 | PCNX3_1 | EGPSGKWSLGGRKGLGGSDGEPASGSPKGGTPKSQAPLD |
| | | LSLSLSLSLSPDVSTEASPPRAS |
| 1627 | RGL4 | PRPGQHALTMPALEPAPPLLADLGPALEPESPAALGPPGYL |
| | | HSAPGPAPAPGEGPPPGTVLE |
| 1628 | SALL3_0 | PVEKEAEPMDAEPAGDTRAPRPPPAAPAPPTPAYGAPSTN |
| | | VTLEALLSTKVAVAQFSQGARA |
| 1629 | SALL3_1 | VPTSVGLQLPPTVPGAHGYADSPSATPASRSPQRPSPASSE |
| | | CASLSPGLNHVESGVSATAES |
| 1630 | SALL3_2 | GLQLPPTVPGAHGYADSPSATPASRSPQRPSPASSECASLS |
| | | PGLNHVESGVSATAESPQSLL |
| 1631 | SREBF1_0 | LQLINNQDSDFPGLFDPPYAGSGAGGTDPASPDTSSPGSLS |
| | | PPPATLSSSLEAFLSGPQAAP |
| 1632 | SREBF1_1 | SPGSLSPPPATLSSSLEAFLSGPQAAPSPLSPPQPAPTPLKM |
| | | YPSMPAFSPGPGIKEESVPL |
| 1633 | SHISA7 | DINVPRALVDILRHQAGPGTRPDRARSSSLTPGIGGPDSMP |
| | | PRTPKNLYNTVKTPNLDWRAL |
| 1634 | KIF24 | LPVSSATRHLWLSSSPPDNKPGGDLPALSPSPIRQHPADKL |
| | | PSREADLGEACQSRETVLFSH |
| 1635 | C4orf54 | PETGQYVDVPMTSQQQAVAPMSISVPPLALSPGAYGPTY |
| | | MIYPGFLPTVLPTNALQPTPIAR |
| 1636 | NPIPB8 | PPSVDDNLKECLFVPLPPSPLPPSVDDNLKTPPLATQEAEV |
| | | EKPPKPKRWRVDEVEQSPKPK |
| 1637 | ATXN2L | LKPQPLQQPSQPQQPPPTQQAVARRPPGGTSPPNGGLPGPL |
| | | ATSAAPPGPPAAASPCLGPVA |
| 1638 | HDAC5 | SKEPTPGGLNHSLPQHPKCWGAHHASLDQSSPPQSGPPGT |
| | | PPSYKLPLPGPYDSRDDFPLRK |
| 1639 | ATF7IP | WKETPCILSVNVKNKQDDDLNCEPLSPHNITPEPVSKLPAE |
| | | PVSGDPAPGDLDAGDPASGVL |
| 1640 | RNF217 | APASEQLSPPASPPGAPPVLNPPSTRSSFPSPRLSLPTDSLSP |
| | | DGGSIELEFYLAPEPFSMP |
| 1641 | ZNF831 | REAPWDSAPMASPGLPAASTQPWRKLPEQKSPTAGKPCA |
| | | LQRQQATAAEKPWDAKAPEGRLR |
| 1642 | CBSL | PEDKEAKEPLWIRPDAPSRCTWQLGRPASESPHHHTAPAK |
| | | SPKILPDILKKIGDTPMVRINK |
| 1643 | INPP5E_0 | PPEGRTLQGQLPGAPPAQRAGSPPDAPGSESPALACSTPAT |
| | | PSGEDPPARAAPIAPRPPARP |
| 1644 | INPP5E_1 | LPGAPPAQRAGSPPDAPGSESPALACSTPATPSGEDPPARA |
| | | APIAPRPPARPRLERALSLDD |
| 1645 | PEX1 | HLGKVWIPDDLRKRLNIEMHAVVRITPVEVTPKIPRSLKLQ |
| | | PRENLPKDISEEDIKTVFYSW |
| 1646 | CAPRIN2 | EEQKKQETPKLWPVQLQKEQDPKKQTPKSWTPSMQSEQN |
| | | TTKSWTTPMCEEQDSKQPETPKS |
| 1647 | CBX4 | RCLSETHGEREPCKKRLTARSISTPTCLGGSPAAERPADLP |
| | | PAAALPQPEVILLDSDLDEPI |
| 1648 | XDH | GDGNNPNCCMNQKKDHSVSLSPSLFKPEEFTPLDPTQEPIF |
| | | PPELLRLKDTPRKQLRFEGER |
| 1649 | EPAS1_0 | ATELRSHSTQSEAGSLPAFTVPQAAAPGSTTPSATSSSSSCS |
| | | TPNSPEDYYTSLDNDLKIEV |
| 1650 | EPAS1_1 | VPNDKFTQNPMRGLGHPLRHLPLPQPPSAISPGENSKSRFP |
| | | PQCYATQYQDYSLSSAHKVSG |
| 1651 | SHANK3_0 | GLVPPPEEFANGVLLATPLAGPGPSPTTVPSPASGKPSSEPP |
| | | PAPESAADSGVEEADTRSSS |
| 1652 | SHANK3_1 | GELTDTHTSFADGHTFLLEKPPVPPKPKLKSPLGKGPVTFR |
| | | DPLLKQSSDSELMAQQHHAAS |
| 1653 | ATF6 | PSAQPVLAVAGGVTQLPNHVVNVVPAPSANSPVNGKLSV |
| | | TKPVLQSTMRNVGSDIAVLRRQQ |
| 1654 | BCOR | KASNPEPSFKANENGLPPSSIFLSPNEAFRSPPIPYPRSYLPY |
| | | PAPEGIAVSPLSLHGKGPV |
| 1655 | CCP110 | SDERGAHIMNSTCAAMPKLHEPYASSQCIASPNFGTVSGL |
| | | KPASMLEKNCSLQTELNKSYDV |
| 1656 | MMP9 | GPPLHKDDVNGIRHLYGPRPEPEPRPPTTTTPQPTAPPTVC |
| | | PTGPPTVHPSERPTAGPTGPP |
| 1657 | BCL11B_0 | LNPMAIDSPAMDFSRRLRELAGNSSTPPPVSPGRGNPMHR |
| | | LLNPFQPSPKSPFLSTPPLPPM |
| 1658 | BCL11B_1 | AGNSSTPPPVSPGRGNPMHRLLNPFQPSPKSPFLSTPPLPPM |
| | | PPGGTPPPQPPAKSKSCEFC |
| 1659 | BCL11B_2 | TPPPVSPGRGNPMHRLLNPFQPSPKSPFLSTPPLPPMPPGGT |
| | | PPPQPPAKSKSCEFCGKTFK |
| 1660 | BCL11B_3 | PMHRLLNPFQPSPKSPFLSTPPLPPMPPGGTPPPQPPAKSKS |
| | | CEFCGKTFKFQSNLIVHRRS |
| 1661 | RB1 | DSIIVFYNSVFMQRLKTNILQYASTRPPTLSPIPHIPRSPYKF |
| | | PSSPLRIPGGNIYISPLKS |
| 1662 | AHSG_0 | GAEVAVTCMVFQTQPVSSQPQPEGANEAVPTPVVDPDAP |
| | | PSPPLGAPGLPPAGSPPDSHVLL |
| 1663 | AHSG_1 | GANEAVPTPVVDPDAPPSPPLGAPGLPPAGSPPDSHVLLA |
| | | APPGHQLHRAHYDLRHTFMGVV |
| 1664 | TCTN3 | TDGGTLQSPSEATATRPAVPGLPTVVPTLVTPSAPGNRTV |
| | | DLFPVLPICVCDLTPGACDINC |
| 1665 | NR2F2 | QDEVPGSQGSQASQAPPVPGPPPGAPHTPQTPGQGGPAST |
| | | PAQTAAGGQGGPGGPGSDKQQQ |
| 1666 | KHDRBS1 | PSVRQTPSRQPPLPHRSRGGGGGSRGGARASPATQPPPLLP |
| | | PSATGPDATVGGPAPTPLLPP |
| 1667 | ARRB1 | SSDVAVELPFTLMHPKPKEEPPHREVPENETPVDTNLIELD |
| | | TNDDDIVFEDFARQRLKGMKD |
| 1668 | TFAP2B | HDGVPSHSSRLSQLGSVSQGPYSSAPPLSHTPSSDFQPPYFP |
| | | PPYQPLPYHQSQDPYSHVND |
| 1669 | KSR2_0 | IQWPTTETGKENNPVCPPEPTPWIRTHLSQSPRVPSKCVQH |
| | | YCHTSPTPGAPVYTHVDRLTV |
| 1670 | KSR2_1 | RSLPPSPRQRHAVRTPPRTPNIVTTVTPPGTPPMRKKNKLK |
| | | PPGTPPPSSRKLIHLIPGFTA |
| 1671 | KSR2_2 | RQQKNFNLPASHYYKYKQQFIFPDVVPVPETPTRAPQVIL |
| | | HPVTSNPILEGNPLLQIEVEPT |
| 1672 | TNS1_0 | SGYIPSGHSLGTPEPAPRASLESVPPGRSYSPYDYQPCLAG |
| | | PNQDFHSKSPASSSLPAFLPT |
| 1673 | TNS1_1 | LPAFLPTTHSPPGPQQPPASLPGLTAQPLLSPKEATSDPSRT |
| | | PEEEPLNLEGLVAHRVAGVQ |
| 1674 | TNS1_2 | SASGYQAPSTPSFPVSPAYYPGLSSPATSPSPDSAAFRQGSP |
| | | TPALPEKRRMSVGDRAGSLP |
| 1675 | ZEB2 | SNSRSPSLERSSKPLAPNSNPPTKDSLLPRSPVKPMDSITSPS |
| | | IAELHNSVTNCDPPLRLTK |
| 1676 | CREBBP_0 | QGQVPGAALPNPLNMLGPQASQLPCPPVTQSPLHPTPPPA |
| | | STAAGMPSLQHTTPPGMTPPQP |
| 1677 | CREBBP_1 | GAALPNPLNMLGPQASQLPCPPVTQSPLHPTPPPASTAAG |
| | | MPSLQHTTPPGMTPPQPAAPTQ |
| 1678 | CREBBP_2 | AQLMRRRMATMNTRNVPQQSLPSPTSAPPGTPTQQPSTPQ |
| | | TPQPPAQPQPSPVSMSPAGFPS |
| 1679 | CREBBP_3 | MATMNTRNVPQQSLPSPTSAPPGTPTQQPSTPQTPQPPAQP |
| | | QPSPVSMSPAGFPSVARTQPP |
| 1680 | CREBBP_4 | MNTRNVPQQSLPSPTSAPPGTPTQQPSTPQTPQPPAQPQPS |
| | | PVSMSPAGFPSVARTQPPTTV |
| 1681 | CREBBP_5 | GQQIATSLSNQVRSPAPVQSPRPQSQPPHSSPSPRIQPQPSP |
| | | HHVSPQTGSPHPGLAVTMAS |
| 1682 | CREBBP_6 | QVRSPAPVQSPRPQSQPPHSSPSPRIQPQPSPHHVSPQTGSP |
| | | HPGLAVTMASSIDQGHLGNP |
| 1683 | CREBBP_7 | APVQSPRPQSQPPHSSPSPRIQPQPSPHHVSPQTGSPHPGLA |
| | | VTMASSIDQGHLGNPEQSAM |
| 1684 | GBF1_0 | IPSELGACDFEKPESPRAASSSSPGSPVASSPSRLSPTPDGPP |
| | | PLAQPPLILQPLASPLQVG |
| 1685 | GBF1_1 | GACDFEKPESPRAASSSSPGSPVASSPSRLSPTPDGPPPLAQ |
| | | PPLILQPLASPLQVGVPPMT |
| 1686 | GBF1_2 | CDFEKPESPRAASSSSPGSPVASSPSRLSPTPDGPPPLAQPPL |
| | | ILQPLASPLQVGVPPMTLP |
| 1687 | ESRP2 | QATPTLIPTETAALYPSSALLPAARVPAAPTPVAYYPGPAT |
| | | QLYLNYTAYYPSPPVSPTTVG |
| 1688 | FGFR1 | PYWTSPEKMEKKLHAVPAAKTVKFKCPSSGTPNPTLRWL |
| | | KNGKEFKPDHRIGGYKVRYATWS |
| 1689 | FNDC1 | IVAMPTTSKADVEQNTEDNGKPEKPEPSSPSPRAPASSQHP |
| | | SVPASPQGRNAKDLLLDLKNK |
| 1690 | LCAT | PWQWVTLLLGLLLPPAAPFWLLNVLFPPHTTPKAELSNHT |
| | | RPVILVPGCLGNQLEAKLDKPD |
| 1691 | COL4A5 | IKGSVGDPGLPGLPGTPGAKGQPGLPGFPGTPGPPGPKGIS |
| | | GPPGNPGLPGEPGPVGGGGHP |
| 1692 | FGD1 | PGQSLEPHPEGPQRLRSDPGPPTETPSQRPSPLKRAPGPKPQ |
| | | VPPKPSYLQMPRMPPPLEPI |
| 1693 | PIK3R1 | IGWLNGYNETTGERGDFPGTYVEYIGRKKISPPTPKPRPPR |
| | | PLPVAPGSSKTEADVEQQALT |
| 1694 | EP300_0 | MQIQRAAETQRQMAHVQIFQRPIQHQMPPMTPMAPMGM |
| | | NPPPMTRGPSGHLEPGMGPTGMQQ |
| 1695 | EP300_1 | GQQIPNSLSNQVRSPQPVPSPRPQSQPPHSSPSPRMQPQPSP |
| | | HHVSPQTSSPHPGLVAAQAN |
| 1696 | EP300_2 | QVRSPQPVPSPRPQSQPPHSSPSPRMQPQPSPHHVSPQTSSP |
| | | HPGLVAAQANPMEQGHFASP |
| 1697 | EP300_3 | QPVPSPRPQSQPPHSSPSPRMQPQPSPHHVSPQTSSPHPGLV |
| | | AAQANPMEQGHFASPDQNSM |
| 1698 | FOXM1_0 | VGGLDFSPVQTSQGASDPLPDPLGLMDLSTTPLQSAPPLES |
| | | PQRLLSSEPLDLISVPFGNSS |
| 1699 | FOXM1_1 | TSQGASDPLPDPLGLMDLSTTPLQSAPPLESPQRLLSSEPLD |
| | | LISVPFGNSSPSDIDVPKPG |
| 1700 | ACD | ICSAPATLTPRSPHASRTPSSPLQSCTPSLSPRSHVPSPHQAL |
| | | VTRPQKPSLEFKEFVGLPC |
| 1701 | SON | SETAETFDSMRASGHVASEVSTSLLVPAVTTPVLAESILEP |
| | | PAMAAPESSAMAVLESSAVTV |
| 1702 | HTT | INICAHVLDDVAPGPAIKAALPSLTNPPSLSPIRRKGKEKEP |
| | | GEQASVPLSPKKGSEASAAS |
| 1703 | PHLPP1 | APGAFGGPPRAPPADLPLPVGGPGGWSRRASPAPSDSSPG |
| | | EPFVGGPVSSPRAPRPVVSDTE |
| 1704 | NAF1 | DFGVGEGPAAPSPGSAPVPGTQPPLQSFEGSPDAGQTVEV |
| | | KPAGEQPLQPVLNAVAAGTPAP |
| 1705 | ERBB2 | PSETDGYVAPLTCSPQPEYVNQPDVRPQPPSPREGPLPAAR |
| | | PAGATLERPKTLSPGKNGVVK |
| 1706 | DAZAP1 | VKFKDPNCVGTVLASRPHTLDGRNIDPKPCTPRGMQPERT |
| | | RPKEGWQKGPRSDNSKSNKIFV |
| 1707 | E2F8 | VAPLDPPVNAEMELTAPSLIQPLGMVPLIPSPLSSAVPLILP |
| | | QAPSGPSYAIYLQPTQAHQS |
| 1708 | PC | RPAQNRAQKLLHYLGHVMVNGPTTPIPVKASPSPTDPVVP |
| | | AVPIGPPPAGFRDILLREGPEG |
| 1709 | TMIGD2 | QSIYSTSFPQPAPRQPHLASRPCPSPRPCPSPRPGHPVSMVR |
| | | VSPRPSPTQQPRPKGFPKVG |
| 1710 | MAPT_0 | PAKTPPAPKTPPSSGEPPKSGDRSGYSSPGSPGTPGSRSRTP |
| | | SLPTPPTREPKKVAVVRTPP |
| 1711 | MAPT_1 | PPSSGEPPKSGDRSGYSSPGSPGTPGSRSRTPSLPTPPTREPK |
| | | KVAVVRTPPKSPSSAKSRL |
| 1712 | MAPT_2 | EPPKSGDRSGYSSPGSPGTPGSRSRTPSLPTPPTREPKKVAV |
| | | VRTPPKSPSSAKSRLQTAPV |
| 1713 | KCNQ2_0 | LIPPLNQLELLRNLKSKSGLAFRKDPPPEPSPSKGSPCRGPL |
| | | CGCCPGRSSQKVSLKDRVFS |
| 1714 | KCNQ2_1 | NQLELLRNLKSKSGLAFRKDPPPEPSPSKGSPCRGPLCGCC |
| | | PGRSSQKVSLKDRVFSSPRGV |
| 1715 | MBNL2 | SFAPYLAPVTPGVGLVPTEILPTTPVIVPGSPPVTVPGSTAT |
| | | QKLLRTDKLEVCREFQRGNC |
| 1716 | FN1 | PGTSGQQPSVGQQMIFEEHGFRRTTPPTTATPIRHRPRPYPP |
| | | NVGEEIQIGHIPREDVDYHL |
| 1717 | KLF5 | TAVKQFQGMPPCTYTMPSQFLPQQATYFPPSPPSSEPGSPD |
| | | RQAEMLQNLTPPPSYAATIAS |
| 1718 | uncharacterized_ | VIRALGPLVPPTEGGLWSDQVSWPLWEDVKTPEPGEPGSP |
| | LOC101060588_0 | LPASPHPPLQPPAFPDPPIRSP |
| 1719 | uncharacterized_ | TPEPGEPGSPLPASPHPPLQPPAFPDPPIRSPDPAVSSAHSFP |
| | LOC101060588_1 | APRLAWSCVLHSPLSLPLS |
| 1720 | translation_initiation_ | PGSLLPTPASLWQAQCPRHMHSWSSAPGRLTPHPPGPAPG |
| | factor_IF-2-like | TKLATGATSSACSRPQGRPCPQ |
| 1721 | putative_uncharacterized_ | SAQAGPPETAHAADPQPRGPQAPPRLPPSLSPERVHPGQPA |
| | protein_MGC34800 | APAEPAPGAPALRSGPSQPRG |
| 1722 | uncharacterized_ | SLPWPLRAAPLYAGRSGQGGEPGARAPRQGTPEPGELDQE |
| | LOC100507221 | RPPAPPEQGRRAAAAVAKSGGG |
| 1723 | basic_proline- | SAGNKENARTWRRSEGGLAGPPLAKAPRSHSPPGCSPHG |
| | rich protein-like_0 | QSLPPRRRTPPSQLTGSARSRRP |
| 1724 | basic_proline- | ENARTWRRSEGGLAGPPLAKAPRSHSPPGCSPHGQSLPPR |
| | rich protein-like_1 | RRTPPSQLTGSARSRRPGSPFR |
| 1725 | basic proline- | RSPGAGGVQGGGAGGIPAPRAPRPPPSGAPSPTHVEPPRPR |
| | rich protein-like_2 | RPAPTREGTRASPHTRASRSR |
| 1726 | uncharacterized_ | CWDSHLPFRKKGAAPAPGCGDRIDTVPTSATPNGRTPGRG |
| | LOC107987269 | ALLAAPILSQPCHFQSCQHPSQ |
| 1727 | sine_oculis- | GCLSKGSQRSLTPSWSPSVSPGSEADSSWGTPSTPPRPHSP |
| | binding_protein_ | PSLPRPSPSPWVQARPGIPPP |
| | homolog_0 | |
| 1728 | sine_oculis- | SPGSEADSSWGTPSTPPRPHSPPSLPRPSPSPWVQARPGIPP |
| | binding_protein_ | PSEQTLFKGLWRLEGIEPPP |
| | homolog_1 | |
| 1729 | mucin-1-like_0 | PAGSPAAPLQTATSVPPWVSSCTTSNCNISSPLGLQQHGPQ |
| | | PGTSAPPNPGLQLHSPQPGTS |
| 1730 | mucin-1-like_1 | NCNISSPLGLQQHGPQPGTSAPPNPGLQLHSPQPGTSAPPN |
| | | PGLQLHGPQTGTSAPCRVSSC |

TABLE 2
List of speckle targeting motif containing proteins according to x(30)-[TSED]P-
x(30) pattern. Proteins with more than one speckle targeting motif are designated by
ProteinName_[0 - number of motifs minus one]. See Table for SEQ ID numbers of
repeated peptides.

| SEQ | | |
|---|---|---|
| ID | | |
| NO: | Name: | Sequence: |
| | MUC17_0 | IPVITSTEASSSPTTAEGTSIPTSTYTEGSTPLTSTPAST |
| | | MPVATSEMSTLSITPVDTSTLV |
| | MUC17_1 | STEASSSPTTAEGTSIPTSTYTEGSTPLTSTPASTMPV |
| | | ATSEMSTLSITPVDTSTLVTTSTE |
| | MUC17_2 | TPVTNSTEARSSPTTSEGTSMPTSTPGEGSTPLTSMP |
| | | DSTTPVVSSEARTLSATPVDTSTPV |
| | MUC17_3 | TQVATSTEASSPPPTAEVTSMPTSTPGERSTPLTSMP |
| | | VRHTPVASSEASTLSTSPVDTSTPV |
| | MUC17_4 | TPVTTSTEACSSPTTSEGTSMPNSNPSEGTTPLTSIPV |
| | | STTPVVSSEASTLSATPVDTSTPG |
| | MUC17_5 | TPGTTSAEATSSPTTAEGISIPTSTPSEGKTPLKSIPVS |
| | | NTPVANSEASTLSTTPVDSNSPV |
| | MUC17_6 | TPVTTSTEARSSPTTSEGTSMPNSTPSEGTTPLTSIPV |
| | | STTPVLSSEASTLSATPIDTSTPV |
| | MUC17_7 | TPVTNSTEARSSPTTSEGTSMPTSTPSEGSTPFTSMPV |
| | | STMPVVTSEASTLSATPVDTSTPV |
| | MUC17_8 | TPVTTYSQAGSSPTTADDTSMPTSTYSEGSTPLTSVP |
| | | VSTMPVVSSEASTHSTTPVDTSTPV |
| | MUC17_9 | TPVTTSTEASSSPTTAEGTSIPTSPPSEGTTPLASMPV |
| | | STTPVVSSEAGTLSTTPVDTSTPM |
| | MUC17_10 | SPVVTSTEISSSATSAEGTSMPTSTYSEGSTPLRSMPV |
| | | STKPLASSEASTLSTTPVDTSIPV |
| | MUC17_11 | IPVTTSTEASSSPTTAEVTSMPTSTPSETSTPLTSMPV |
| | | NHTPVASSEAGTLSTTPVDTSTPV |
| | MUC17_12 | TPVTTSTEASSSPTTAEGTGIPISTPSEGSTPLTSIPVST |
| | | TPVAIPEASTLSTTPVDSNSPV |
| | MUC17_13 | SPVVTSTEVSSSPTPAEGTSMPISTYSEGSTPLTGVPV |
| | | STTPVTSSAISTLSTTPVDTSTPV |
| | MUC17_14 | STPVTTSTEATSSTTAEGTSIPTSTPSEGMTPLTSVPV |
| | | SNTPVASSEASILSTTPVDSNTPL |
| | MUC17_15 | TPVTTSTEASLSPTTAEGTSIPTSSPSEGTTPLASMPV |
| | | STTPVVSSEVNTLSTTPVDSNTLV |
| | MUC17_16 | TLVTTSTEASSSPTIAEGTSLPTSTTSEGSTPLSIMPLS |
| | | TTPVASSEASTLSTTPVDTSTPV |
| | MUC17_17 | TPVTTSSPTNSSPTTAEVTSMPTSTAGEGSTPLTNMP |
| | | VSTTPVASSEASTLSTTPVDSNTFV |
| 1731 | TGOLN2 | HAFKTESGEETDLISPPQEEVKSSEPTEDVEPKEAED |
| | | DDTGPEEGSPPKEEKEKMSGSASSE |
| | MYO15B_0 | HRLALRLAGLAGLGGMPRASPGGRSPQVPTSPVPG |
| | | DPFDQEDETPDPKFAVVFPRIHRAGRA |
| | MYO15B_1 | AFLRKIDPKDEALAKLGINGAHSSPPMLSPSPGKGPP |
| | | PAVAPRPKAPLQLGPSSSIKEKQGP |
| | FAM178B | RPCSPASAPAPTSPKKPKIQAPGETFPTDWSPPPVEFL |
| | | NPRVLQASREAPAQRWVGVVGPQG |
| 1732 | INPP5J_0 | GSPPCIQTSPDPRLSPSFRARPEALHSSPEDPVLPRPP |
| | | QTLPLDVGQGPSEPGTHSPGLLSP |
| 1733 | INPP5J_1 | RPEALHSSPEDPVLPRPPQTLPLDVGQGPSEPGTHSP |
| | | GLLSPTFRPGAPSGQTVPPPLPKPP |
| | INPP5J_2 | HSSPEDPVLPRPPQTLPLDVGQGPSEPGTHSPGLLSP |
| | | TFRPGAPSGQTVPPPLPKPPRSPSR |
| | INPP5J_3 | DPVLPRPPQTLPLDVGQGPSEPGTHSPGLLSPTFRPG |
| | | APSGQTVPPPLPKPPRSPSRSPSHS |
| | COL15A1 | VAEILEAVTYTQASPKEAKVEPINTPPTPSSPFEDME |
| | | LSGEPVPEGTLETTNMSIIQHSSPK |
| | SH3RF1 | PTAAARISELSGLSCSAPSQVHISTTGLIVTPPPSSPVT |
| | | TGPSFTFPSDVPYQAALGTLNPP |
| 1734 | FMN2 | GAGEAPGSPDTEQALSALSDLPESLAAEPREPQQPPS |
| | | PGGLPVSEAPSLPAAQPAAKDSPSS |
| | EZHIP | DENPSCGTGSERLAFQSRSGSPDPEVPSRASPPVWH |
| | | AVRMRASSPSPPGRFFLPIPQQWDES |
| | CTAGE1 | EFKIKLLEKDPYGLDVPNTAFGRQHSPYGPSPLGWP |
| | | SSETRASLYPPTLLEGPLRLSPLLPR |
| | BPTF_0 | PTHAQSSKPQVAAQSQPQSNVQGQSPVRVQSPSQTR |
| | | IRPSTPSQLSPGQQSQVQTTTSQPIP |
| | BPTF_1 | QPQSNVQGQSPVRVQSPSQTRIRPSTPSQLSPGQQSQ |
| | | VQTTTSQPIPIQPHTSLQIPSQGQP |
| | NRXN3 | KMNNRDLKPQPDIVLLPLPTAYELDSTKLKSPLITSP |
| | | MFRNVPTANPTEPGIRRVPGASEVI |
| | ANKHD1-EIF4EBP3 | PHFALLAAQTMQQIRHPRLPMAQFGGTFSPSPNTW |
| | | GPFPVRPVNPGNTNSSPKHNNTSRLPN |
| | putative_UPF0607_protein_ | LCLIPRNTGTPQRVLRPVVWSPPSRKKPVLSPHNSIM |
| | FLJ37424 | FGHLSPVRIPCLRGKFNLQLPSLDD |
| 1735 | C1orf94_0 | KVPDNKNVLDKTRVTKDFLQDNLFSGPGPKEPTGL |
| | | SPFLLLPPRPPPARPDKLPELPAQKRQ |
| | C1orf94_1 | KNVLDKTRVTKDFLQDNLFSGPGPKEPTGLSPFLLL |
| | | PPRPPPARPDKLPELPAQKRQLPVFA |
| | ITIH6_0 | KPGSLSHQNPDILPTNSRTQVPPVKPGIPASPKADTV |
| | | KCVTPLHSKPGAPSHPQLGALTSQA |
| | ITIH6_1 | LSKTPKILLSLKPSAPPHQISTSISLSKPETPNPHMPQT |
| | | PLPPRPDRPRPPLPESLSTFPNT |
| | KIAA1614 | GSINEEQPARDGGPRLPRPPAPGREYCNRGSPWPPE |
| | | AEWTLPDHDRGPLLGPSSLQQSPIHG |
| | KRTAP10-10 | CTDSWRVVDCPESCCEPCCCAPAPSLTLVCTPVSCV |
| | | SSPCCQTACEPSACQSGYTSSCTTPC |
| | IFITM10 | LGDPASTTDGAQEARVPLDGAFWIPRPPAGSPKGCF |
| | | ACVSKPPALQAPAAPAPEPSASPPMA |
| | MS4A15 | GLCPPPAILPTSMCQPPGIMQFEEPPLGAQTPRATQP |
| | | PDLRPVETFLTGEPKVLGTVQILIG |
| 1736 | SP5_0 | HSPLALLAATCSRIGQPGAAAPPDFLQVPYDPALGS |
| | | PSRLFHPWTADMPAHSPGALPPPHPS |
| | SP5_1 | PQKTHLQPSFGAAHELPLTPPADPSYPYEFSPVKMLP |
| | | SSMAALPASCAPAYVPYAAQAALPP |
| | FOXE1 | AARPPYPGAVYAGYAPPSLAAPPPVYYPAASPGPCR |
| | | VFGLVPERPLSPELGPAPSGPGGSCA |
| | PRICKLE2 | EYAWVPPGLKPEQVHQYYSCLPEEKVPYVNSPGEK |
| | | LRIKQLLHQLPPHDNEVRYCNSLDEEE |
| | C7orf26_0 | LCTRDDLRTLCSRLPHNNLLQLVISGPVQQSPHAAL |
| | | PPGFYPHIHTPPLGYGAVPAHPAAHP |
| | C7orf26_1 | HNNLLQLVISGPVQQSPHAALPPGFYPHIHTPPLGYG |
| | | AVPAHPAAHPALPTHPGHTFISGVT |
| | MAGEB17_0 | EKRRQARGEDQCLGGAQATAAEKEKLPSSSSPACQ |
| | | SPPQSFPNAGIPQESQRASYPSSPASA |
| | MAGEB17_1 | ARGEDQCLGGAQATAAEKEKLPSSSSPACQSPPQSF |
| | | PNAGIPQESQRASYPSSPASAVSLTS |
| | ATP6V1FNB | ARLPLKLPTLHPKAPLSPPPAPKSAPSKVPSPVPEAPF |
| | | QSEMYPVPPITRALLYEGISHDFQ |
| | PCDH9 | ATDGGQPPRSSTAKVTINVMDVNDNSPVVISPPSNT |
| | | SFKLVPLSAIPGSVVAEVFAVDVDTG |
| | FAM131C | YLQDSLPSGPSQDDSLQAFSSPSPSPDSCPSPEEPPST |
| | | AGIPQPPSPELQHRRRLPGAQGPE |
| | FAM221B | SAEDLQENHISESFLKPSTSETPLEPHTSESPLVPSPSQ |
| | | IPLEAHSPETHQEPSISETPSET |
| 1737 | TOX3_0 | FFAASEQTFHTPSLGDEEFEIPPITPPPESDPALGMPD |
| | | VLLPFQALSDPLPSQGSEFTPQFP |
| | TOX3_1 | QQLQQQLQQRLQLQQLQHMQHQSQPSPRQHSPVAS |
| | | QITSPIPAIGSPQPASQQHQSQIQSQT |
| | MAMSTR | EQISDPDPWISASDPPLAPALPSGTAPFLFSPGVLLPE |
| | | PEYCPPWRSPKKESPKISQRWRES |
| | ZAN | CAQAGQAPAWRNRTFCPMRCPPGSSYSPCSSPCPDT |
| | | CSSINNPRDCPKALPCAESCECQKGH |
| | PCLO_0 | RPQTKQADIVRGESVKPSLPSPSKPPIQQPTPGKPPA |
| | | QQPGHEKSQPGPAKPPAQPSGLTKP |
| | PCLO_1 | KTPAQQPGPAKPPTQQVGTPKPLAQQPGLQSPAKAP |
| | | GPTKTPVQQPGPGKIPAQQAGPGKTS |
| | PCLO_2 | KPPTQQVGTPKPLAQQPGLQSPAKAPGPTKTPVQQP |
| | | GPGKIPAQQAGPGKTSAQQTGPTKPP |
| 1738 | PCLO_3 | TSAVSKSSPQPQQTSPKKDAAPKQDLSKAPEPKKPP |
| | | PLVKQPTLHGSPSAKAKQPPEADSLS |
| 1739 | IER5L | RGQPLEPLQPGPAPLPLPLPPPAPAALCPRDPRAPAA |
| | | CSAPPGAAPPAAAASPPASPAPASS |
| | C22orf23 | IMDIMKRGDALPLQCSPTSSQRVLPSKQIASPIYLPPI |
| | | LAARPHLRPANMCQANGAYSREQF |
| | HSFX1 | RNSRGQDHGLERVPFPPQLQSETYLHPADPSPAWD |
| | | DPGSTGSPNLRLLTEEIAFQPLAEEAS |
| | FAM13C | RNLLCEQPTVPRENGKPEAAGPEPSSSGEETPDAAL |
| | | TCLKERREQLPPQEDSKVTKQDKNLI |
| | THAP8_0 | PLQKNTPLPQSPAIPVSGPVRLVVLGPTSGSPKTVAT |
| | | MLLTPLAPAPTPERSQPEVPAQQAQ |
| | THAP8_1 | SPAIPVSGPVRLVVLGPTSGSPKTVATMLLTPLAPAP |
| | | TPERSQPEVPAQQAQTGLGPVLGAL |
| 1740 | PRR27_0 | PPLPPRGFPFVPPSRFFSAAAAPAAPPIAAEPAAAAPL |
| | | TATPVAAEPAAGAPVAAEPAAEAP |
| | PRR27_1 | VPPSRFFSAAAAPAAPPIAAEPAAAAPLTATPVAAEP |
| | | AAGAPVAAEPAAEAPVGAEPAAEAP |
| 1741 | PRR27_2 | FFSAAAAPAAPPIAAEPAAAAPLTATPVAAEPAAGA |
| | | PVAAEPAAEAPVGAEPAAEAPVAAEP |
| 1742 | PRR27_3 | PPIAAEPAAAAPLTATPVAAEPAAGAPVAAEPAAEA |
| | | PVGAEPAAEAPVAAEPAAEAPVGVEP |
| 1743 | PRR27_4 | APLTATPVAAEPAAGAPVAAEPAAEAPVGAEPAAE |
| | | APVAAEPAAEAPVGVEPAAEEPSPAEP |
| 1744 | PRR27_5 | EPAAGAPVAAEPAAEAPVGAEPAAEAPVAAEPAAE |
| | | APVGVEPAAEEPSPAEPATAKPAAPEP |
| 1745 | PRR27_6 | EPAAEAPVGAEPAAEAPVAAEPAAEAPVGVEPAAE |
| | | EPSPAEPATAKPAAPEPHPSPSLEQAN |
| 1746 | RINL | DTPGKVLSIVNQLYLETHRGWGREQTPQETEPEAA |
| | | QRHDPAPRNPAPHGVSWVKGPLSPEVD |
| | LRRN4 | VLEPDISAASTPLASKLLGPFPTSWDRSISSPQPGQRT |
| | | HATPQAPNPSLSEGEIPVLLLDDY |
| | KDF1 | QRLKSTMGSSFSYPDVKLKGIPVYPYPRATSPAPDA |
| | | DSCCKEPLADPPPMRHSLPSTFASSP |
| 1747 | FNDC10 | PDVHDSVLYRLCLQPLPLRAGPAAAAPETPEPAECV |
| | | EFTAEPAGMQDIVVAMTAVGGSICVM |
| 1748 | C1QL1_0 | SGAPPPSTLVQGPQGKPGRTGKPGPPGPPGDPGPPGP |
| | | VGPPGEKGEPGKPGPPGLPGAGGSG |
| 1749 | C1QL1_1 | KPGRTGKPGPPGPPGDPGPPGPVGPPGEKGEPGKPG |
| | | PPGLPGAGGSGAISTATYTTVPRVAF |
| | NEXMIF | INGVKENDSEDQDVAMKSFAALEAAAPIQPTPVAQ |
| | | KETLMYPRGLLPLPSKKPCMQSPPSPL |
| | KLHDC7B | PGGGWPWVSREVPGTRSFGPAPDSTRPWLESPPQG |
| | | RPLSSQGPGATGAYDAGEAGADSSRDN |
| 1750 | C19orf67_0 | TEQWFEGSLPLDPGETPPPDALEPGTPPCGDPSRSTP |
| | | PGRPGNPSEPDPEDAEGRLAEARAS |
| | C19orf67_1 | EGSLPLDPGETPPPDALEPGTPPCGDPSRSTPPGRPG |
| | | NPSEPDPEDAEGRLAEARASTSSPK |
| 1751 | CYSRT1 | RPDLGQQLEVASTCSSSSEMQPLPVGPCAPEPTHLL |
| | | QPTEVPGPKGAKGNQGAAPIQNQQAW |
| | RAB44_0 | TAHSELPQQDSLLVSLPSATPQAQVEAEGPTPGKSA |
| | | PPRGSPPRGAQPGAGAGPQEPTQTPP |
| | RAB44_1 | SLLVSLPSATPQAQVEAEGPTPGKSAPPRGSPPRGAQ |
| | | PGAGAGPQEPTQTPPTMAEQEAQPR |
| 1752 | C16orf90 | LGPRNSLCSALLEARLPRDSLGSSASSSSMDPDKGA |
| | | LPQPSPSRLRPKRSWGTWEEAMCPLC |
| | ZNF341_0 | SGTVEIQALGMQPYPPLEVPNQCVEPPVYPTPTVYS |
| | | PGKQGFKPKGPNPAAPMTSATGGTVA |
| | ZNF341_1 | IQALGMQPYPPLEVPNQCVEPPVYPTPTVYSPGKQG |
| | | FKPKGPNPAAPMTSATGGTVATFDSP |
| 1753 | RTL10_0 | EQQLTKESTPGPKEPPVLPSSTCSSKPGPVEPASSQPE |
| | | EAAPTPVPRLSESANPPAQRPDPA |
| | RTL10_1 | KEPPVLPSSTCSSKPGPVEPASSQPEEAAPTPVPRLSE |
| | | SANPPAQRPDPAHPGGPKPQKTEE |
| 1754 | BNIP5 | PLCVGGHRPSTSSSLDPEDLECREPLPAEGEPVVISE |
| | | APSQARGHTPEGAPQLSGACESKEI |
| | IQCN_0 | KTLLQTYPVVSVTLPQTYPASTMTTTPPKTSPVPKV |
| | | TIIKTPAQMYPGPTVTKTAPHTCPMP |
| | IQCN_1 | SVTLPQTYPASTMTTTPPKTSPVPKVTIIKTPAQMYP |
| | | GPTVTKTAPHTCPMPTMTKIQVHPT |
| 1755 | FREM3 | TPRQLLVALACLLLSRPALQGRASSLGTEPDPALYL |
| | | PARGALDGTRPDGPSVLIANPGLRVP |
| | ZNF653 | SPVGSSGLITQEGVHIPFDVHHVESLAEQGTPLCSNP |
| | | AGNGPEALETVVCVPVPVQVGAGPS |
| | KRTAP10-11 | QVDDCPESCCEPPCSAPSCCAPAPSLSLVCTPVSCVS |
| | | SPCCQAACEPSACQSGCTSSCTPSC |
| 1756 | TTBK1_0 | VPLAEEEDFDSKEWVIIDKETELKDFPPGAEPSTSGT |
| | | TDEEPEELRPLPEEGEERRRLGAEP |
| 1757 | TTBK1_1 | SKEWVIIDKETELKDFPPGAEPSTSGTTDEEPEELRPL |
| | | PEEGEERRRLGAEPTVRPRGRSMQ |
| | TTBK1_2 | TNSLPNGPALADGPAPVSPLEPSPEKVATISPRRHAM |
| | | PGSRPRSRIPVLLSEEDTGSEPSGS |
| | CCDC184 | GRDPEDEEEEEEEKEMPSPATPSSHCERPESPCAGLL |
| | | GGDGPLVEPLDMPDITLLQLEGEAS |
| 1758 | PRR15 | GPWWKSLTNSRKKSKEAAVGVPPPAQPAPGEPTPP |
| | | APPSPDWTSSSRENQHPNLLGGAGEPP |
| 1759 | LAMB4_0 | GQHCDRCRPLFYRDPLKTISDPYACIPCECDPDGTIS |
| | | GGICVSHSDPALGSVAGQCLCKENV |
| 1760 | LAMB4_1 | SVAGQCLCKENVEGAKCDQCKPNHYGLSATDPLGC |
| | | QPCDCNPLGSLPFLTCDVDTGQCLCLS |
| | UBQLN3_0 | QSLGTYLQGTASALSQSQEPPPSVNRVPPSSPSSQEP |
| | | GSGQPLPEESVAIKGRSSCPAFLRY |
| 1761 | UBQLN3_1 | YLQGTASALSQSQEPPPSVNRVPPSSPSSQEPGSGQP |
| | | LPEESVAIKGRSSCPAFLRYPTENS |
| | UBQLN3_2 | SSTGHSTNLPDLVSGLGDSANRVPFAPLSFSPTAAIP |
| | | GIPEPPWLPSPAYPRSLRPDGMNPA |
| 1762 | UBQLN3_3 | DLVSGLGDSANRVPFAPLSFSPTAAIPGIPEPPWLPSP |
| | | AYPRSLRPDGMNPAPQLQDEIQPQ |
| 1763 | C10orf82 | VLQHEELLPKYPDFSIPDGSCPALGRPLREDPKTPLT |
| | | CGCAQRPSIPCSGKMYLEPLSSAKY |
| | PRDM8 | STPAAASPVGAEKLLAPRPGGPLPSRLEGGSPARGS |
| | | AFTSVPQLGSAGSTSGGGGTGAGAAG |
| | PCBP4 | GTPSSAPADLPAPFSPPLTALPTAPPGLLGTPYAISLS |
| | | NFIGLKPMPFLALPPASPGPPPGL |
| 1764 | MARVELD3 | GARGLTWDAAAPPGPAPWEAPEPPQPQRKGDPGRR |
| | | RPESEPPSERYLPSTPRPGREEVEYYQ |
| | RNF222_0 | KSSQTLAVPVGLPSVPPLDSLGHTNPLAASSPAWRP |
| | | PPGQARPPGSPGQSAQLPLDLLPSLP |
| | RNF222_1 | PPLDSLGHTNPLAASSPAWRPPPGQARPPGSPGQSA |
| | | QLPLDLLPSLPRESQIFVISRHGMPL |
| 1765 | PARP8 | QVVDLLVSMCRSALESPRKVVIFEPYPSVVDPNDPQ |
| | | MLAFNPRKKNYDRVMKALDSITSIRE |
| | ARMCX5 | ARYIVLVPVEGGEQSLPPEGNWTLVETLIETPLGIRP |
| | | LTKIPPYHGPYYQTLAEIKKQIRQR |
| | DNM1 | RPGSRGPAPGPPPAGSALGGAPPVPSRPGASPDPFGP |
| | | PPQVPSRPNRAPPGVPSRSGQASPS |
| 1766 | PIANP | TPSGFEEGPPSSQYPWAIVWGPTVSREDGGDPNSAN |
| | | PGFLDYGFAAPHGLATPHPNSDSMRG |
| 1767 | KCP | LNGREHRSGEPVGSGDPCSHCRCANGSVQCEPLPCP |
| | | PVPCRHPGKIPGQCCPVCDGCEYQGH |
| | ZNF541 | EACGDSPHAHESAGQPPPSSLRSLVPPEARSPGSLLP |
| | | HRDLLRRIVSSIVHQKTPSPGPAPA |
| 1768 | PCDHA9 | VCSGEGKQKTDLMAFSPGLSPCAGSTERTGEPSASS |
| | | DSTGKPRQPNPDWRYSASLRAGMHSS |
| 1769 | DMRTB1 | AAACFFEQPPRGRNPGPRALQPVLGGRSHVEPSERA |
| | | AVAMPSLAGPPFGAEAAGSGYPGPLD |
| | FMN1_0 | PAPAALGKVFNNSASQSSTHKQTSPVPSPLSPRLPSP |
| | | QQHHRILRLPALPGEREAALNDSPC |
| | FMN1_1 | LGKVFNNSASQSSTHKQTSPVPSPLSPRLPSPQQHHR |
| | | ILRLPALPGEREAALNDSPCRKSRV |
| | FBXO41 | LFARKSVASSACSTPPPGPGPGPCPGPASASPASPSP |
| | | ADVAYEEGLARLKIRALEKLEVDRR |
| | GAS2L2 | TKASLSAKGTHMRKVPPQGGQDCSASTVSASPEAP |
| | | TPSPLDPNSDKAKACLSKGRRTLRKPK |
| 1770 | ZAR1L | QPDWRQNMGPPTFLARPGLLVPANAPDYCIDPYKR |
| | | AQLKAILSQMNPSLSPRLCKPNTKEVG |
| 1771 | SHPK | WGYFNTQSQSWNVETLRSSGFPVHLLPDIAEPGSVA |
| | | GRTSHMWFEIPKGTQVGVALGDLQAS |
| | UBAP1L | VSRPRALLHGLRGHRALSLCPSPAQSPRSASPPGPAP |
| | | QHPAAPASPPRPSTAGAIPPLRSHK |
| | IGSF9B_0 | PFHHGQYYGYLSSSSPGEVEPPPFYVPEVGSPLSSVM |
| | | SSPPLPTEGPFGHPTIPEENGENAS |
| | IGSF9B_1 | NSTLPLTQTPTGGRSPEPWGRPEFPFGGLETPAMMF |
| | | PHQLPPCDVPESLQPKAGLPRGLPPT |
| | ATF7-NPFF | GCGMVVGTASTMVTARPEQSQILIQHPDAPSPAQPQ |
| | | VSPAQPTPSTGGRRRRTVDEDPDERR |
| | HSFX2 | RNSRGQDHGLERVPFPPQLQSETYLHPADPSPAWD |
| | | DPGSTGSPNLRLLTEEIAFQPLAEEAS |
| 1772 | TMEM108 | QGGTPDATAASGAPVSPQAAPVPSQRPHHGDPQDG |
| | | PSHSDSWLTVTPGTSRPLSTSSGVFTA |
| 1773 | NT5C1B-RDH14 | LRKTDSRGYLVRSQWSRISRSPSTKAPSIDEPRSRNT |
| | | SAKLPSSSTSSRTPSTSPSLHDSSP |
| | NPIPB6 | PPSVDDNLKECLFVPLPPSPLPPSVDDNLKTPPLATQ |
| | | EAEVEKPPKPKRWRVDEVEQSPKPK |
| | PCED1B | HSDVPSSAHAGFFVEDNFMVGPQLPMPFFPTPRYQR |
| | | PAPVVHRGFGRYRPRGPYTPWGQRPR |
| 1774 | FIGNL2 | QLEPFEKFPERAPAPRGGFAVPSGETPKGVDPGALE |
| | | LVTSKMVDCGPPVQWADVAGQGALKA |
| | NPIPB9 | PPSVDDNLKECLFVPLPPSPLPPSVDDNLKTPPLATQ |
| | | EAEVEKPPKPKRWRVDEVEQSPKPK |
| | SLFNL1 | DLLLSEAQGPFSHREEKEEEEEDSGLSPGPSPGSGVP |
| | | LPTWPTHTLPDRPQAQQLQSCQGRP |
| | NLGN4Y | HNLNEIFQYVSTTTKVPPPDMTSFPYGTRRSPAKIWP |
| | | TTKRPAITPANNPKHSKDPHKTGPE |
| | PRRT4 | VALPLALLGLYPALCSPRVPPRCWAKLFRLSPGHAA |
| | | PLLPGGWVTGPPDKEPLGSAIARGDA |
| 1775 | NUTM1_0 | ASALPGPDMSMKPSAAPSPSPALPFLPPTSDPPDHPP |
| | | REPPPQPIMPSVFSPDNPLMLSAFP |
| | NUTM1_1 | PALPFLPPTSDPPDHPPREPPPQPIMPSVFSPDNPLML |
| | | SAFPSSLLVTGDGGPCLSGAGAGK |
| 1776 | LMTK3_0 | TPFSPEGAFPGGGAAEEEGVPRPRAPPEPPDPGAPRP |
| | | PPDPGPLPLPGPREKPTFVVQVSTE |
| | LMTK3_1 | VSENGGLRFPRNTERPPETGPWRAPGPWEKTPESW |
| | | GPAPTIGEPAPETSLERAPAPSAVVSS |
| | LMTK3_2 | PTNELSVQAPPEGDTDPSTPPAPPTPPHPATPGDGFP |
| | | SNDSGFGGSFEWAEDFPLLPPPGPP |
| | ZCCHC14 | SSLNGGGGHGGKGAPGPGGALPTCPACHKITPRTEA |
| | | PVSSVSNSLENALHTSAHSTEESLPK |
| | MIA2 | ELKFELLEKDPYALDVPNTAFGREHSPYGPSPLGWP |
| | | SSETRAFLSPPTLLEGPLRLSPLLPG |
| | CTNND2_0 | AAAAAALYYSSSTLPAPPRGGSPLAAPQGGSPTKLQ |
| | | RGGSAPEGATYAAPRGSSPKQSPSRL |
| 1777 | CTNND2_1 | AESSGCWGKKKKKKKSQDQWDGVGPLPDCAEPPK |
| | | GIQMLWHPSIVKPYLTLLSECSNPDTLE |
| 1778 | CPXCR1 | EGSDTAGNAHKNSENEPPNDCSTDIESPSADPNMIY |
| | | QVETNPINREPGTATSQEDVVPQAAE |
| | NRG3 | SRTPNRISTRLTTITRAPTRFPGHRVPIRASPRSTTAR |
| | | NTAAPATVPSTTAPFFSSSTLGSR |
| | KCNC2 | KTLPGTRLALLASSEPPGDCLTTAGDKLQPSPPPLSP |
| | | PPRAPPLSPGPGGCFEGGAGNCSSR |
| 1779 | SEMA6B | PGRASHGDFPLTPHASPDRRRVVSAPTGPLDPASAA |
| | | DGLPRPWSPPPTGSLRRPLGPHAPPA |
| 1780 | LRRC56 | QDWLAVKEAIKKGNGLPPLDCPRGAPIRRLDPELSL |
| | | PETQSRASRPWPFSLLVRGGPLPEGL |
| 1781 | DNAJB13 | DDRLLNIPINDIIHPKYFKKVPGEGMPLPEDPTKKGD |
| | | LFIFFDIQFPTRLTPQKKQMLRQAL |
| | CD300E_0 | DAGSYWCKIQTVWVLDSWSRDPSDLVRVYVSPAIT |
| | | TPRRTTHPATPPIFLVVNPGRNLSTGE |
| | CD300E_1 | WCKIQTVWVLDSWSRDPSDLVRVYVSPAITTPRRTT |
| | | HPATPPIFLVVNPGRNLSTGEVLTQN |
| | COL9A1 | SVPFELQWMLIHCDPLRPRRETCHELPARITPSQTTD |
| | | ERGPPGEQGPPGPPGPPGVPGIDGI |
| | HTR3E | TIFITHLLHVATTQPPPLPRWLHSLLLHCNSPGRCCP |
| | | TAPQKENKGPGLTPTHLPGVKEPEV |
| 1782 | ZNF385C_0 | TLASGAPGEPQSKVPAAPPLGPPLQPPPTPDPTCREP |
| | | AHSELLDAASSSSSSSCPPCSPEPG |
| 1783 | ZNF385C_1 | APGEPQSKVPAAPPLGPPLQPPPTPDPTCREPAHSEL |
| | | LDAASSSSSSSCPPCSPEPGREAPG |
| | NPIPB15 | PPSVDDNLKDCLFVPLPPSPLPPSVDDNLKTPPLATQ |
| | | EAEAEKPPKPKRWRVDEVEQSPKPK |
| | SPEM3 | HLVRSSVPVPTSAPAPPGTLAPATTPVLAPTPAPVPA |
| | | SAPSPAPALVMALTTTPVPDPVPAT |
| | KRTAP10-4 | QVDDCPESCCEPPCCAPSCCAPAPCLSLVCTPVSRVS |
| | | SPCCPVTCEPSPCQSGCTSSCTPSC |
| 1784 | LMLN | VSIQMNGWIHDGNLLCPSCWDFCELCPPETDPPATN |
| | | LTRALPLDLCSCSSSLVVTLWLLLGN |
| | CRIP3 | GVNIGGVGSYLYNPPTPSPGCTTPLSPSSFSPPRPRTG |
| | | LPQGKKSPPHMKTFTGETSLCPGC |
| | LRRC37A2 | PEHSHLTQATVQPLDLGFTITPESKTEVELSPTMKET |
| | | PTQPPKKVVPQLRVYQGVTNPTPGQ |
| 1785 | FER1L6 | DVEPPPTVVPDSAQAQPAILVDVPDSSPMLEPEHTP |
| | | VAQEPPKDGKPKDPRKPSRRSTKRRK |
| 1786 | ZGLP1 | GCVACPRVHKEPAQVGTPWPAKPRSHPRKRDPTAL |
| | | LPRSLWPACQESVTALCFLQETVERLG |
| | KRTAP10-6 | CSDSWQVDDCPESCCEPPCCAPAPCLSLVCTPVSRV |
| | | SSPCCPVTCEPSPCQSGCTSSCTPSC |
| | PNMA5 | GRSMTDVARALGCCSLPAESLDAEVMPQVRSPPLEP |
| | | PKESMWYRKLKVFSGTASPSPGEETF |
| | ZNF683 | LLPYPGAFQASGQALPSQARNPGAGAAPTDSPGLER |
| | | GGMASPAKRVPLSSQTGTAALPYPLK |
| | PRR23A | CALAPNPSSEGHSPGPFFDPEFRLLEPVPSSPLQPLPP |
| | | SPRVGSPGPHAHPPLPKRPPCKAR |
| | SELENOV_0 | PTPLRTPTPVRTRTPIRTLTPVLTPSPAGTSPLVLTPA |
| | | PAQIPTLVPTPALARIPRLVPPPA |
| | SELENOV_1 | TPTPVRTRTPIRTLTPVLTPSPAGTSPLVLTPAPAQIPT |
| | | LVPTPALARIPRLVPPPAPAWIP |
| | SELENOV_2 | ALARIPRLVPPPAPAWIPTPVPTPVPVRNPTPVPTPAR |
| | | TLTPPVRVPAPAPAQLLAGIRAAL |
| 1788 | SELENOV_3 | LPVLDSYLAPALPLDPPPEPAPELPLLPEEDPEPAPSL |
| | | KLIPSVSSEAGPAPGPLPTRTPLA |
| | STON1-GTF2A1L_0 | EFPSGSSSTSSTPLSSPIVDFYFSPGPPSNSPLSTPTKD |
| | | FPGFPGIPKAGTHVLYPIPESSS |
| | STON1-GTF2A1L_1 | ISGGESSLLPTRPTCLSHALLPSDHSCTHPTPKVGLPD |
| | | EVNPQQAESLGFQSDDLPQFQYFR |
| | POC1B-GALNT4 | AVVVVTGRRCRSGQTVPGAARSPLLPHPLPSPLRVP |
| | | PPTGALGRPLPRWPQPRRTPFWSVIS |
| 1789 | IKZF5_0 | KPFMIQQPSTQAVVSAVSASIPQSSSPTSPEPRPSHSQ |
| | | RNYSPVAGPSSEPSAHTSTPSIGN |
| | IKZF5_1 | PTSPEPRPSHSQRNYSPVAGPSSEPSAHTSTPSIGNSQ |
| | | PSTPAPALPVQDPQLLHHCQHCDM |
| | RHBDD3 | SCGYMPVHLAMLAGEGHRPRRPRGALPPWLSPWLL |
| | | LALTPLLSSEPPFLQLLCGLLAGLAYA |
| 1790 | SATL1 | QLGMRQPGTSQSSKNQTGMSHPGRGQPGIWEPGPS |
| | | QPGLSQQDLNQLVLSQPGLSQPGRSQP |
| 1791 | PRR23C_0 | PIRGPCALAPNPSSERRSPRPIFDLEFHLLEPVPSSPLQ |
| | | PLPPSPSPGPHARPELPERPPCK |
| | PRR23C_1 | CALAPNPSSERRSPRPIFDLEFHLLEPVPSSPLQPLPPS |
| | | PSPGPHARPELPERPPCKVRRRL |
| | PRR23B | CALAPNPSSERRSPRPIFDLEFRLLEPVPSSPLQPLPPS |
| | | PCVGSPGPHARSPLPERPPCKAR |
| | STRC | GSNRRLVKRLCAGLLPPPTSCPEGLPPVPLTPDIFWG |
| | | CFLENETLWAERLCGEASLQAVPPS |
| 1792 | KRTAP29-1 | CLPSSCHSRMWQLVTCQESCQPSIGAPSGCDPASCQ |
| | | PTRLPATSCVGFVCQPMCSHAACYQS |
| 1793 | PRKCSH | YDRVWAAIRDKYRSEALPTDLPAPSAPDLTEPKEEQ |
| | | PPVPSSPTEEEEEEEEEEEEEAEEEE |
| 1794 | B3GNT8 | VYIEWTSESRLSKAYPSPRGTPPSPTPANPEPTLPAN |
| | | LSTRLGQTIPLPFAYWNQQQWRLGS |
| | NKX1-1_0 | NPGADTSAPTGGGGGPGPGAGPGTGLPGGLSPLSPS |
| | | PPMGAPLGMHGPAGYPAHGPGGLVCA |
| | NKX1-1_1 | TSAPTGGGGGPGPGAGPGTGLPGGLSPLSPSPPMGA |
| | | PLGMHGPAGYPAHGPGGLVCAAQLPF |
| | HCFC1R1 | ATHFSQLSLHNDHPYCSPPMTFSPALPPLRSPCSELL |
| | | LWRYPGSLIPEALRLLRLGDTPSPP |
| | SPATA31A3_0 | SLSASQPPEPSLPLEHPSPEPPALFPHPPHTPDPLACSL |
| | | PPPKGFTAPPLRDSTLITPSHCD |
| 1795 | SPATA31A3_1 | SASQPPEPSLPLEHPSPEPPALFPHPPHTPDPLACSLPP |
| | | PKGFTAPPLRDSTLITPSHCDSV |
| | OTUD4 | TCTDAHFPMQTEASVNGQMPQPEIGPPTFSSPLVIPP |
| | | SQVSESHGQLSYQADLESETPGQLL |
| | LRRC37A | PEHSHLTQATVQPLDLGFTITPESKTEVELSPTMKET |
| | | PTQPPKKVVPQLRVYQGVTNPTPGQ |
| | FOXB2 | PEYGAFGVPVKSLCHSASQSLPAMPVPIKPTPALPPV |
| | | SALQPGLTVPAASQQPPAPSTVCSA |
| | KRTAP10-8 | SPSTCTGSSWQVDNCQESCCEPRSCASSCCTPSCCAP |
| | | APCLALVCAPVSCEPSPCQSGCTDS |
| 1796 | PVRIG | SPCANTTFCCKFASFPEGSWEACGSLPPSSDPGLSAP |
| | | PTPAPILRADLAGILGVSGVLLFGC |
| | KRTAP10-12 | CSDSWQVDDCPESCCEPPCCAPAPCLSLVCTPVSRV |
| | | SSPCCRVTCEPSPCQSGCTSSCTPSC |
| | PLAGL2 | PPGATGGLVMGYSQAEAQPLLTTLQAQPQDSPGAG |
| | | GPLNFGPLHSLPPVFTSGLSSTTLPRF |
| | CCDC187_0 | AGQACSPQRAWGAQRQGPSSQRPGSPPEKRSPFPQQ |
| | | PWSAVATQPCPRRAWTACETWEDPGP |
| | CCDC187_1 | DTVRDPAVGLLRSCPHSLPAAPTLATPTLATPACPG |
| | | ALGPNWGRGAPGEWVSMQPQPLLPPT |
| 1797 | KRTAP2-1 | TCQTTVCRPVTCVPRCTRPICEPCRRPVCCDPCSLQE |
| | | GCCRPITCCPSSCTAVVCRPCCWAT |
| | SPATA31A7_0 | SLSASQPPEPSLPLEHPSPEPPALFPHPPHTPDPLACSL |
| | | PPPKGFTAPPLRDSTLITPSHCD |
| 1798 | SPATA31A7_1 | SASQPPEPSLPLEHPSPEPPALFPHPPHTPDPLACSLPP |
| | | PKGFTAPPLRDSTLITPSHCDSV |
| | NOBOX | LEELEPQDYQQSNQPGPFQFSQAPQPPLFQSPQPKLP |
| | | YLPTFPFSMPSSLTLPPPEDSLFMF |
| | TTN_0 | LSATSSAQKITKSVKAPTVKPSETRVRAEPTPLPQFP |
| | | FADTPDTYKSEAGVEVKKEVGVSIT |
| | TTN_1 | PAAPLGAPTYIPTLEPVSRIRSLSPRSVSRSPIRMSPAR |
| | | MSPARMSPARMSPARMSPGRRLE |
| | TTN_2 | GAPTYIPTLEPVSRIRSLSPRSVSRSPIRMSPARMSPA |
| | | RMSPARMSPARMSPGRRLEETDES |
| | TTN_3 | IPTLEPVSRIRSLSPRSVSRSPIRMSPARMSPARMSPA |
| | | RMSPARMSPGRRLEETDESQLERL |
| | TTN_4 | PVSRIRSLSPRSVSRSPIRMSPARMSPARMSPARMSP |
| | | ARMSPGRRLEETDESQLERLYKPVF |
| | TTN_5 | RSLSPRSVSRSPIRMSPARMSPARMSPARMSPARMS |
| | | PGRRLEETDESQLERLYKPVFVLKPV |
| | TTN_6 | RSVSRSPIRMSPARMSPARMSPARMSPARMSPGRRL |
| | | EETDESQLERLYKPVFVLKPVSFKCL |
| 1799 | TTN_7 | EYEPTEEYDQYEEYEEREYERYEEHEEYITEPEKPIP |
| | | VKPVPEEPVPTKPKAPPAKVLKKAV |
| 1800 | TTN_8 | YEEREYERYEEHEEYITEPEKPIPVKPVPEEPVPTKPK |
| | | APPAKVLKKAVPEEKVPVPIPKKL |
| | TTN_9 | PEVPPTKVPEVPKAAVPEKKVPEAIPPKPESPPPEVPE |
| | | APKEVVPEKKVPAAPPKKPEVTPV |
| | TTN_10 | PEVPPTKVPEVPKVAVPEKKVPEAIPPKPESPPPEVFE |
| | | EPEEVALEEPPAEVVEEPEPAAPP |
| 1801 | TTN_11 | PKPESPPPEVFEEPEEVALEEPPAEVVEEPEPAAPPQV |
| | | TVPPKKPVPEKKAPAVVAKKPELP |
| 1801 | TTN_12 | PEEEIAPEEEKPVPVAEEEEPEVPPPAVPEEPKKIIPEK |
| | | KVPVIKKPEAPPPKEPEPEKVIE |
| 1803 | TTN_13 | CSVEKLIEGHEYQFRICAENKYGVGDPVFTEPAIAK |
| | | NPYDPPGRCDPPVISNITKDHMTVSW |
| | TTN_14 | IELMRPVSELIRSRPQPAEEYEDDTERRSPTPERTRPR |
| | | SPSPVSSERSLSRFERSARFDIFS |
| | TTN_15 | EKAVTSPPRVKSPEPRVKSPEAVKSPKRVKSPEPSHP |
| | | KAVSPTETKPTPTEKVQHLPVSAPP |
| | TTN_16 | KSPEPRVKSPEAVKSPKRVKSPEPSHPKAVSPTETKP |
| | | TPTEKVQHLPVSAPPKITQFLKAEA |
| | KIF26B | ESDKEDNGSEGQLTNREGPELPASKMQRSHSPVPAA |
| | | APAHSPSPASPRSVPGSSSQHSASPL |
| 1804 | ZNF114 | TFPEANRVCLTSISSQHSTLREDWRCPKTEEPHRQG |
| | | VNNVKPPAVAPEKDESPVSICEDHEM |
| 1805 | COL16A1_0 | DGGIKGVPGKPGRDGRPGEICVIGPKGQKGDPGFVG |
| | | PEGLAGEPGPPGLPGPPGIGLPGTPG |
| 1806 | COL16A1_1 | EKGNFGEAGPAGSPGPPGPVGPAGIKGAKGEPCEPC |
| | | PALSNLQDGDVRVVALPGPSGEKGEP |
| | COL16A1_2 | NSGEKGDQGFQGQPGFPGPPGPPGFPGKVGSPGPPG |
| | | PQAEKGSEGIRGPSGLPGSPGPPGPP |
| | ESAM_0 | DTISKNGTLSSVTSARALRPPHGPPRPGALTPTPSLSS |
| | | QALPSPRLPTTDGAHPQPISPIPG |
| | ESAM_1 | TSARALRPPHGPPRPGALTPTPSLSSQALPSPRLPTTD |
| | | GAHPQPISPIPGGVSSSGLSRMGA |
| | DUSP8_0 | QLLEYERSLKLLAALQGDPGTPSGTPEPPPSPAAGAP |
| | | LPRLPPPTSESAATGNAAAREGGLS |
| | DUSP8_1 | DIKSAYAPSRRPDGPGPPDPGEAPKLCKLDSPSGAA |
| | | LGLSSPSPDSPDAAPEARPRPRRRPR |
| | DUSP8_2 | RPDGPGPPDPGEAPKLCKLDSPSGAALGLSSPSPDSP |
| | | DAAPEARPRPRRRPRPPAGSPARSP |
| | DUSP8_3 | GPPDPGEAPKLCKLDSPSGAALGLSSPSPDSPDAAPE |
| | | ARPRPRRRPRPPAGSPARSPAHSLG |
| | DUSP8_4 | PRHGLSALSAPGLPGPGQPAGPGAWAPPLDSPGTPS |
| | | PDGPWCFSPEGAQGAGGVLFAPFGRA |
| | DUSP8_5 | SALSAPGLPGPGQPAGPGAWAPPLDSPGTPSPDGPW |
| | | CFSPEGAQGAGGVLFAPFGRAGAPGP |
| 1807 | BEST4 | QPQPPYTVATAAESLRPSFLGSTFNLRMSDDPEQSL |
| | | QVEASPGSGRPAPAAQTPLLGRFLGV |
| | SULT1A2 | KCHRAPIFMRVPFLEFKVPGIPSGMETLKNTPAPRLL |
| | | KTHLPLALLPQTLLDQKVKVVYVAR |
| 1808 | LRTM2 | SSAGLDIPGPPCTKASPEPAKPKPGAEPEPEPSTACPQ |
| | | KQRHRPASVRRAMGTVIIAGVVCG |
| | GPR150 | TVLGVACGHLLSVWWRHRPQAPAAAAPWSASPGR |
| | | APAPSALPRAKVQSLKMSLLLALLFVGC |
| | DRAP1 | SEDTDTDGEEETSQPPPQASHPSAHFQSPPTPFLPFAS |
| | | TLPLPPAPPGPSAPDEEDEEDYDS |
| | IQCE | FRGHLTRTKLLASKAHGSEPPSVPGLPDQSSPVPRVP |
| | | SPIAQATGSPVQEEAIVIIQSALRA |
| 1809 | COL14A1 | CSCSETNEVALGPAGPPGGPGLRGPKGQQGEPGPKG |
| | | PDGPRGEIGLPGPQGPPGPQGPSGLS |
| | SOX13 | INLLQQQIQQVNMPYVMIPAFPPSHQPLPVTPDSQLA |
| | | LPIQPIPCKPVEYPLQLLHSPPAPV |
| 1810 | RALGDS | AVGLESAPAPALELEPAPEQDPAPSQTLELEPAPAPV |
| | | PSLQPSWPSPVVAENGLSEEKPHLL |
| | CEP170B_0 | QDFMAQCLRESSPAARPSPEKVPPVLPAPLTPHGTSP |
| | | VGPPTPPPAPTDPQLTKARKQEEDD |
| | CEP170B_1 | QCLRESSPAARPSPEKVPPVLPAPLTPHGTSPVGPPT |
| | | PPPAPTDPQLTKARKQEEDDSLSDA |
| | MAGEC2 | STSSSLILGGPEEEEVPSGVIPNLTESIPSSPPQGPPQG |
| | | PSQSPLSSCCSSFSWSSFSEESS |
| | COL22A1 | GLPGLKGDRGEKGEAGPAGPPGLPGTTSLFTPHPRM |
| | | PGEQGPKGEKGDPGLPGEPGLQGRPG |
| 1811 | SH3RF2 | LTCISRGSEAWIHSAASSLIMEDKEIPIKSEPLPKPPAS |
| | | APPSILVKPENSRNGIEKQVKTV |
| 1812 | SPRR4 | PPQRAQQQQVKQPCQPPPVKCQETCAPKTKDPCAP |
| | | QVKKQCPPKGTIIPAQQKCPSAQQASK |
| 1813 | EFCAB6_0 | MDDDQYALLTTKIGFEKEGMSYLDFAAGFEDPPMR |
| | | GPETTPPQPPTPSKSYVNSHFITAEEC |
| | EFCAB6_1 | EKEGMSYLDFAAGFEDPPMRGPETTPPQPPTPSKSY |
| | | VNSHFITAEECLKLFPRRLKESFRDP |
| 1814 | DDN | AQLAGLPAPLRPERLAPVGRAPRPSAQPQSDPGSAW |
| | | AGPWGGRRPGPPSYEAHLLLRGSAGT |
| | BEND4 | PNPSSASEYGHLADVDPLSTSPVHTLGGWTSPATSE |
| | | SHGHPSSSTLPEEEEEEDEEGYCPRC |
| | ATRIP | LKVLVKLAENTSCDFLPRFQCVFQVLPKCLSPETPLP |
| | | SVLLAVELLSLLADHDQLAPQLCSH |
| | NCAN | NRVEAHGEATATAPPSPAAETKVYSLPLSLTPTGQG |
| | | GEAMPTTPESPRADFRETGETSPAQV |
| 1815 | SYNE4_0 | EESTSPEQAQTLGQDSLGPPEHFQGGPRGNEPAAHP |
| | | PRWSTPSSYEDPAGGKHCEHPISGLE |
| | SYNE4_1 | TLGQDSLGPPEHFQGGPRGNEPAAHPPRWSTPSSYE |
| | | DPAGGKHCEHPISGLEVLEAEQNSLH |
| 1816 | ATAT1_0 | FVIFEGFFAHQHRPPAPSLRATRHSRAAAVDPTPAAP |
| | | ARKLPPKRAEGDIKPYSSSDREFLK |
| | ATAT1_1 | DIKPYSSSDREFLKVAVEPPWPLNRAPRRATPPAHPP |
| | | PRSSSLGNSPERGPLRPFVPEQELL |
| | ATAT1_2 | AVEPPWPLNRAPRRATPPAHPPPRSSSLGNSPERGPL |
| | | RPFVPEQELLRSLRLCPPHPTARLL |
| | TESK1_0 | KIKLLDTPSKPVLPLVPPSPFPSTQLPLVTTPETLVQP |
| | | GTPARRCRSLPSSPELPRRMETAL |
| 1817 | TESK1_1 | RRMETALPGPGPPAVGPSAEEKMECEGSSPEPEPPG |
| | | PAPQLPLAVATDNFISTCSSASQPWS |
| 1818 | TESK1_2 | VVVNSPQGWAGEPWNRAQHSLPRAAALERTEPSPP |
| | | PSAPREPDEGLPCPGCCLGPFSFGFLS |
| 1819 | TMEM221 | PAEVSKASPRAQPQQGIHRRTPYSTCPEPGDPFGSM |
| | | ATATAPAALEGGWESSLPASRMHRTL |
| | MYBPHL | AAGSKLKVKEASPADAEPPQASPGQGAGSPTPQLLP |
| | | PIEEHPKIWLPRALRQTYIRKVGDTV |
| | DENND2C | SEDNIYEDIIYPTKENPYEDIPVQPLPMWRSPSAWKL |
| | | PPAKSAFKAPKLPPKPQFLHRKTME |
| 1820 | GALNT12 | GLGSVLRAQRGAGAGAAEPGPPRTPRPGRREPVMP |
| | | RPPVPANALGARGEAVRLQLQGEELRL |
| 1821 | CLNK | GDASVRKNKIPLPPPRPLITLPKKYQPLPPEPESSRPP |
| | | LSQRHTFPEVQRMPSQISLRDLSE |
| | PTPN4_0 | DHMVHTSPSEVFVNQRSPSSTQANSIVLESSPSQETP |
| | | GDGKPPALPPKQSKKNSWNQIHYSH |
| | PTPN4_1 | TSPSEVFVNQRSPSSTQANSIVLESSPSQETPGDGKPP |
| | | ALPPKQSKKNSWNQIHYSHSQQDL |
| | MYCL_0 | HYFYDYDCGEDFYRSTAPSEDIWKKFELVPSPPTSPP |
| | | WGLGPGAGDPAPGIGPPEPWPGGCT |
| 1822 | MYCL_1 | TAPSEDIWKKFELVPSPPTSPPWGLGPGAGDPAPGIG |
| | | PPEPWPGGCTGDEAESRGHSKGWGR |
| | FAM110A_0 | PCRRPQLDLDILSSLIDLCDSPVSPAEASRTPGRAEG |
| | | AGRPPPATPPRPPPSTSAVRRVDVR |
| | FAM110A_1 | GAGRPPPATPPRPPPSTSAVRRVDVRPLPASPARPCP |
| | | SPGPAAASSPARPPGLQRSKSDLSE |
| | SSC5D_0 | VCAGQRVANSRDDSTSPLDGAPWPGLLLELSPSTEE |
| | | PLVTHAPRPAGNPQNASRKKSPRPKQ |
| | SSC5D_1 | TAGKLGPTLGAGTTRSPGSPPTLRVHGDTGSPRKPW |
| | | PERRPPRPAATRTAPPTPSPGPSASP |
| | SSC5D_2 | NPDLILTSPDFALSTPDSSVVPALTPEPSPTPLPTLPKE |
| | | LTSDPSTPSEVTSLSPTSEQVPE |
| | SSC5D_3 | PALESSPSRSSTATSMDPLSTEDFKPPRSQSPNLTPPP |
| | | THTPHSASDLTVSPDPLLSPTAHP |
| | SSC5D_4 | STATSMDPLSTEDFKPPRSQSPNLTPPPTHTPHSASD |
| | | LTVSPDPLLSPTAHPLDHPPLDPLT |
| | SSC5D_5 | TEDFKPPRSQSPNLTPPPTHTPHSASDLTVSPDPLLSP |
| | | TAHPLDHPPLDPLTLGPTPGQSPG |
| | SSC5D_6 | SDLTVSPDPLLSPTAHPLDHPPLDPLTLGPTPGQSPG |
| | | PHGPCVAPTPPVRVMACEPPALVEL |
| 1823 | STARD9_0 | SPQRLCSKHMPQLHSIFLSWDPSTTLPPRPDPTHQTS |
| | | EKTSSEEHLPQAASYPARTGCLRKN |
| 1824 | STARD9_1 | QPCSSQPVATHAYSSHSSTLLCFRDGDLGKEPFKAA |
| | | PHTIHPPCVVPSRAYEMDETGEISRG |
| | PTPRN_0 | GGVVNVGADIKKTMEGPVEGRDTAELPARTSPMPG |
| | | HPTASPTSSEVQQVPSPVSSEPPKAAR |
| | PTPRN_1 | RDTAELPARTSPMPGHPTASPTSSEVQQVPSPVSSEP |
| | | PKAARPPVTPVLLEKKSPLGQSQPT |
| | SOX30_0 | PTTVYPYRSPTYSVVIPSLQNPITHPVGETSPAIQLPT |
| | | PAVQSPSPVTLFQPSVSSAAQVAV |
| | SOX30_1 | HARFATSTIQPPREYSSVSPCPRSAPIPQASPIPHPHV |
| | | YQPPPLGHPATLFGTPPRFSFHHP |
| | CSPG4_0 | AGRVTYGATARASEAVEDTFRFRVTAPPYFSPLYTF |
| | | PIHIGGDPDAPVLTNVLLVVPEGGEG |
| 1825 | CSPG4_1 | RNKTGKHDVQVLTAKPRNGLAGDTETFRKVEPGQ |
| | | AIPLTAVPGQGPPPGGQPDPELLQFCRT |
| | RP1L1 | SPQVSLGDGQSEEASESSSPVPEDRPTPPPSPGGDTP |
| | | HQRPGSQTGPSSSRASSWGNCWQKD |
| 1826 | PRELP | QPTRRPRPGTGPGRRPRPRPRPTPSFPQPDEPAEPTD |
| | | LPPPLPPGPPSIFPDCPRECYCPPD |
| | C3orf22 | DSNTVQLPLQKRLVPTRSIPVRGLGAPDFTSPSGSCP |
| | | APLPAPSPPPLCNLWELKLLSRRFP |
| | COL19A1_0 | GIGIPGRTGAQGPAGEPGIQGPRGLPGLPGTPGTPGN |
| | | DGVPGRDGKPGLPGPPGDPIALPLL |
| | COL19A1_1 | SQGERGKPGLTGMKGAIGPMGPPGNKGSMGSPGHQ |
| | | GPPGSPGIPGIPADAVSFEEIKKYINQ |
| | KCNH5 | QLLSCRMTALEKQVAEILKILSEKSVPQASSPKSQMP |
| | | LQVPPQIPCQDIFSVSRPESPESDK |
| | FAM110D | QVIARRQEPALRGSPGPLTPHPCNELGPPASPRTPRP |
| | | VRRGSGRRLPRPDSLIFYRQKRDCK |
| | RUSC1 | HELAQKRKRGPGLPLVPQAKKDRSDWLIVFSPDTEL |
| | | PPSGSPGGSSAPPREVTTFKELRSRS |
| | PCARE_0 | RKASPTRTHWVPQADKRRRSLPSSYRPAQPSPSAVQ |
| | | TPPSPPVSPRVLSPPTTKRRTSPPHQ |
| | PCARE_1 | ADKRRRSLPSSYRPAQPSPSAVQTPPSPPVSPRVLSPP |
| | | TTKRRTSPPHQPKLPNPPPESAPA |
| | PCARE_2 | KVSGNTHSIFCPATSSLFEAKPPLSTAHPLTPPSLPPE |
| | | AGGPLGNPAECWKNSSGPWLRADS |
| | RASSF7 | AALGCEPRKTLTPEPAPSLSRPGPAAPVTPTPGCCTD |
| | | LRGLELRVQRNAEELGHEAFWEQEL |
| | MAN2B1 | ALGFSTYSVAQVPRWKPQARAPQPIPRRSWSPALTI |
| | | ENEHIRATFDPDTGLLMEIMNMNQQL |
| | EPX | RRPLLGASNQALARWLPAEYEDGLSLPFGWTPSRR |
| | | RNGFLLPLVRAVSNQIVRFPNERLTSD |
| | NCCRP1_0 | EVREGHALGGGMEADGPASLQELPPSPRSPSPPPSPP |
| | | PLPSPPSLPSPAAPEAPELPEPAQP |
| | NCCRP1_1 | GMEADGPASLQELPPSPRSPSPPPSPPPLPSPPSLPSPA |
| | | APEAPELPEPAQPSEAHARQLLL |
| | NCCRP1_2 | PASLQELPPSPRSPSPPPSPPPLPSPPSLPSPAAPEAPEL |
| | | PEPAQPSEAHARQLLLEEWGPL |
| | EMILIN2 | RGLPRGVDGQTGSGTVPGAEGFAGAPGYPKSPPVA |
| | | SPGAPVPSLVSFSAGLTQKPFPSDGGV |
| 1828 | STAC3 | TLRTGVIMANKERKKGQADKKNPVAAMMEEEPES |
| | | ARPEEGKPQDGNPEGDKKAEKKTPDDKH |
| | LMOD1 | GNTDTKKDDEKVKKNEPLHEKEAKDDSKTKTPEKQ |
| | | TPSGPTKPSEGPAKVEEEAAPSIFDEP |
| | MYBPC2_0 | GKDAPKGAPKEAPPKEAPAEAPKEAPPEDQSPTAEE |
| | | PTGVFLKKPDSVSVETGKDAVVVAKV |
| 1829 | MYBPC2_1 | KGAPKEAPPKEAPAEAPKEAPPEDQSPTAEEPTGVF |
| | | LKKPDSVSVETGKDAVVVAKVNGKEL |
| | MAGI2_0 | TSAPSSEKQSPMAQQSPLAQQSPLAQPSPATPNSPIA |
| | | QPAPPQPLQLQGHENSYRSEVKARQ |
| | MAGI2_1 | DEPAPWSSPAAAAPGLPEVGVSLDDGLAPFSPSHPA |
| | | PPSDPSHQISPGPTWDIKREHDVRKP |
| | MAGI2_2 | LPEVGVSLDDGLAPFSPSHPAPPSDPSHQISPGPTWDI |
| | | KREHDVRKPKELSACGQKKQRLGE |
| 1830 | RPP25L | DSWVPASPDTGLDPLTVRRHVPAVWVLLSRDPLDP |
| | | NECGYQPPGAPPGLGSMPSSSCGPRSR |
| 1831 | IGDCC3 | RDEKRVDMKELEQLFPPASAAGQPDPRPTQDPAAP |
| | | APCEETQLSVLPLQGCGLMEGKTTEAK |
| | RTN2 | LDLRLRLAQPSSPEVLTPQLSPGSGTPQAGTPSPSRS |
| | | RDSNSGPEEPLLEEEEKQWGPLERE |
| | TP53BP2 | QGKPGSPEPETEPVSSVQENHENERIPRPLSPTKLLPF |
| | | LSNPYRNQSDADLEALRKKLSNAP |
| | HCN1_0 | PPVYTATSLSHSNLHSPSPSTQTPQPSAILSPCSYTTA |
| | | VCSPPVQSPLAARTFHYASPTASQ |
| | HCN1_1 | PTASQLSLMQQQPQQQVQQSQPPQTQPQQPSPQPQT |
| | | PGSSTPKNEVHKSTQALHNTNLTREV |
| | HCN1_2 | LSLMQQQPQQQVQQSQPPQTQPQQPSPQPQTPGSST |
| | | PKNEVHKSTQALHNTNLTREVRPLSA |
| | HCN1_3 | QQPQQQVQQSQPPQTQPQQPSPQPQTPGSSTPKNEV |
| | | HKSTQALHNTNLTREVRPLSASQPSL |
| | TRIM10 | NERPARELLTDIRSTLIRCETRKCRKPVAVSPELGQR |
| | | IRDFPQQALPLQREMKMFLEKLCFE |
| | KCNH4_0 | VSQLSRELRHIMGLLQARLGPPGHPAGSAWTPDPPC |
| | | PQLRPPCLSPCASRPPPSLQDTTLAE |
| 1832 | KCNH4_1 | EVHCPASVGTMETGTALLDLRPSILPPYPSEPDPLGP |
| | | SPVPEASPPTPSLLRHSFQSRSDTF |
| 1833 | MEGF9_0 | VASAASAGNVTGGGGAAGQVDASPGPGLRGEPSHP |
| | | FPRATAPTAQAPRTGPPRATVHRPLAA |
| | MEGF9_1 | APTTLSTTTGPAPTTPVATTVPAPTTPRTPTPDLPSSS |
| | | NSSVLPTPPATEAPSSPPPEYVCN |
| 1834 | COL24A1_0 | EPGYPGDKGAVGLPGPPGMRGKSGPSGQTGDPGLQ |
| | | GPSGPPGPEGFPGDIGIPGQNGPEGPK |
| | COL24A1_1 | LPGIRGGPGRTGLAGAPGPPGVKGSSGLPGSPGIQGP |
| | | KGEQGLPGQPGIQGKRGHRGAQGDQ |
| 1835 | IGSF21 | FSRYQAQNFTLVCIVSGGKPAPMVYFKRDGEPIDAV |
| | | PLSEPPAASSGPLQDSRPFRSLLHRD |
| 1836 | COL27A1 | VAGERGHLGSRGFPGIPGPSGPPGTKGLPGEPGPQGP |
| | | QGPIGPPGEMGPKGPPGAVGEPGLP |
| | PLA2G3 | GTVPLARLQPRTFYNASWSSRATSPTPSSRSPAPPKP |
| | | RQKQHLRKGPPHQKGSKRPSKANTT |
| 1837 | FRS3_0 | DDHRRGRHCLQPLPEGQAPFLPQARGPDQRDPQVF |
| | | LQPGQVKFVLGPTPARRHMVKCQGLCP |
| | FRS3_1 | DETPLQKPTSTRAAIRSHGSFPVPLTRRRGSPRVFNF |
| | | DFRRPGPEPPRQLNYIQVELKGWGG |
| | NYNRIN | PSLSEEILRCLSLHDPPDGALDIDLLPGAASPYLGIPW |
| | | DGKAPCQQVLAHLAQLTIPSNFTA |
| | MBD6_0 | NAPSYNWGAALRSSLVPSDLGSPPAPHASSSPPSDPP |
| | | LFHCSDALTPPPLPPSNNLPAHPGP |
| | MBD6_1 | VPSDLGSPPAPHASSSPPSDPPLFHCSDALTPPPLPPS |
| | | NNLPAHPGPASQPPVSSATMHLPL |
| | MBD6_2 | ASHSSSLRPSQRRPRRPPTVFRLLEGRGPQTPRRSRP |
| | | RAPAPVPQPFSLPEPSQPILPSVLS |
| 1838 | MBD6_3 | FRLLEGRGPQTPRRSRPRAPAPVPQPFSLPEPSQPILP |
| | | SVLSLLGLPTPGPSHSDGSFNLLG |
| | MBD6_4 | PSLPGTTSGSLSSVPGAPAPPAASKAPVVPSPVLQSP |
| | | SEGLGMGAGPACPLPPLAGGEAFPF |
| | MBD6_5 | TTSGSLSSVPGAPAPPAASKAPVVPSPVLQSPSEGLG |
| | | MGAGPACPLPPLAGGEAFPFPSPEQ |
| 1839 | MBD6_6 | ACLLQSLQIPPEQPEAPCLPPESPASALEPEPARPPLS |
| | | ALAPPHGSPDPPVPELLTGRGSGK |
| | MBD6_7 | APCLPPESPASALEPEPARPPLSALAPPHGSPDPPVPE |
| | | LLTGRGSGKRGRRGGGGLRGINGE |
| | PRR35_0 | LYNHMKYSLCKDSLSLLLDSPDWACRRGSTTPRPH |
| | | APTPDRPGESDPGRQPQGARPTGAAPA |
| 1840 | PRR35_1 | LLLDSPDWACRRGSTTPRPHAPTPDRPGESDPGRQP |
| | | QGARPTGAAPAPDLVVADIHSLHCGG |
| | PRR35_2 | AAAHVPFLASASPLLPPATAFPAVQPPQRPTPAPRLY |
| | | YPLLLEHTLGLPAGKAALAKAPVSP |
| | PRR35_3 | SLTRFCSRSSLPTGSSVMLWPEDGDPGGPETPGPEGP |
| | | LPLQPRGPVPGSPEHVGEDLTRALG |
| 1841 | LMNTD2_0 | RASSEQALVQAGSYSRDSEDLQKTHSPRHGEPVLSP |
| | | QPCTDPDHWSPELLQSPTGLKIVAVS |
| 1842 | LMNTD2_1 | AGSYSRDSEDLQKTHSPRHGEPVLSPQPCTDPDHWS |
| | | PELLQSPTGLKIVAVSCREKFVRIFN |
| 1843 | LMNTD2_2 | SGKLFHAREGPARPENPEIPAPQHLPAIPGDPTLPSPP |
| | | AEAGLGLEDCRLQKEHRVRVCRKS |
| | CACNA1D | LMQQQIMAVAGLDSSKAQKYSPSHSTRSWATPPAT |
| | | PPYRDWTPCYTPLIQVEQSEALDQVNG |
| | ORAI3 | FSTALGTFLFLAEVVLVGWVKFVPIGAPLDTPTPMV |
| | | PTSRVPGTLAPVATSLSPASNLPRSS |
| | FOXE3 | GPPLPFPYAPYAPAPGPALLVPPPSAGPGPSPPARLFS |
| | | VDSLVNLQPELAGLGAPEPPCCAA |
| | POM121C_0 | SSPAAPAASSASPMFKPIFTAPPKSEKEGLTPPGPSVS |
| | | ATAPSSSSLPTTTSTTAPTFQPVF |
| | POM121C_1 | AADFSGFGSTLATSAPATSSQPTLTFSNTSTPTFNIPF |
| | | GSSAKSPLPSYPGANPQPAFGAAE |
| | MMP24 | LQGIQKIYGPPAEPLEPTRPLPTLPVRRIHSPSERKHE |
| | | RQPRPPRPPLGDRPSTPGTKPNIC |
| | GPR162 | PPRGPGFFREEITTFIDETPLPSPTASPGHSPRRPRPLG |
| | | LSPRRLSLGSPESRAVGLPLGLS |
| | ZMIZ1_0 | GNPMANANNPMNPGGNPMASGMTTSNPGLNSPQF |
| | | AGQQQQFSAKAGPAQPYIQQSMYGRPNY |
| | ZMIZ1_1 | YSNYSQGNVNRPPRPVPVANYPHSPVPGNPTPPMTP |
| | | GSSIPPYLSPSQDVKPPFPPDIKPNM |
| 1844 | DOK7_0 | EGEQISFLFDCIVRGISPTKGPFGLRPVLPDPSPPGPST |
| | | VEERVAQEALETLQLEKRLSLLS |
| 1845 | DOK7_1 | PSGWLGTRRRGLVMEAPQGSEATLPGPAPGEPWEA |
| | | GGPHAGPPPAFFSACPVCGGLKVNPPP |
| 1846 | TMEM79 | STVSEAATLPWGTGPQPSAPFPDPPGWRDIEPEPPES |
| | | EPLTKLEELPEDDANLLPEKAARAF |
| 1847 | ZFHX2 | GQEPPTHGPEPTPSRDQAAEGPNLTPEASPDPLPEPP |
| | | LASVEVPDKPSGSPGQPPSPAPSPV |
| | ADAMTSL5 | FQARVQALGWPLRQPQPRGVEPQPPAAPAVTPAQT |
| | | PTLAPDPCPPCPDTRGRAHRLLHYCGS |
| | PPP2R3A | AVLIQQTPEVIKIQNKPEKKPGTPLPPPATSPSSPRPL |
| | | SPVPHVNNVVNAPLSINIPRFYFP |
| | PCDH8 | SPEEAARGAGPRPNMFDVLTFPGTGKAPFGSPAADA |
| | | PPPAVAAAEVPGSEGGSATGESACHF |
| | MMP25 | LYGKAPQTPYDKPTRKPLAPPPQPPASPTHSPSFPIPD |
| | | RCEGNFDAIANIRGETFFFKGPWF |
| | COL5A3_0 | GRKKNKEIWTSSPPPDSAENQTSTDIPKTETPAPNLP |
| | | PTPTPLVVTSTVTTGLNATILERSL |
| | COL5A3_1 | SSPPPDSAENQTSTDIPKTETPAPNLPPTPTPLVVTST |
| | | VTTGLNATILERSLDPDSGTELGT |
| | COL5A3_2 | FPGPKGGPGDPGPTGLKGDKGPPGPVGANGSPGER |
| | | GPLGPAGGIGLPGQSGSEGPVGPAGKK |
| | COL5A3_3 | DPGPPGPIGSLGHPGPPGVAGPLGQKGSKGSPGSMG |
| | | PRGDTGPAGPPGPPGAPAELHGLRRR |
| | SOX7 | PLHCSHPLGSLALGQSPGVSMMSPVPGCPPSPAYYS |
| | | PATYHPLHSNLQAHLGQLSPPPEHPG |
| | SEZ6L_0 | IVASEEASEVPLWLDRKESAVPTTPAPLQISPFTSQP |
| | | YVAHTLPQRPEPGEPGPDMAQEAPQ |
| 1848 | SEZ6L_1 | VPTTPAPLQISPFTSQPYVAHTLPQRPEPGEPGPDMA |
| | | QEAPQEDTSPMALMDKGENELTGSA |
| | VGF | GSQQGPEEEAAEALLTETVRSQTHSLPAPESPEPAAP |
| | | PRPQTPENGPEASDPSEELEALASL |
| | PRR30 | LSPHQGLPPSQPPFSSTQSRRPSSPPPASPSPGFQFGSC |
| | | DSNSDFAPHPYSPSLPSSPTFFH |
| | SOBP | ASTTVSPSDTANCSVTKIPTPVPKSIPISETPNIPPVSV |
| | | QPPASIGPPLGVPPRSPPMVMTN |
| | INO80B_0 | LKLKIKLGGQVLGTKSVPTFTVIPEGPRSPSPLMVVD |
| | | NEEEPMEGVPLEQYRAWLDEDSNLS |
| 1849 | INO80B_1 | VLGTKSVPTFTVIPEGPRSPSPLMVVDNEEEPMEGVP |
| | | LEQYRAWLDEDSNLSPSPLRDLSGG |
| | INO80B_2 | PMVRYCSGAQGSTLSFPPGVPAPTAVSQRPSPSGPPP |
| | | RCSVPGCPHPRRYACSRTGQALCSL |
| 1850 | POU5F1_0 | MAGHLASDFAFSPPPGGGGDGPGGPEPGWVDPRTW |
| | | LSFQGPPGGPGIGPGVGPGSEVWGIPP |
| | POU5F1_1 | YAQREDFEAAGSPFSGGPVSFPLAPGPHFGTPGYGS |
| | | PHFTALYSSVPFPEGEAFPPVSVTTL |
| | POU5F1_2 | DFEAAGSPFSGGPVSFPLAPGPHFGTPGYGSPHFTAL |
| | | YSSVPFPEGEAFPPVSVTTLGSPMH |
| 1851 | EMILIN3 | TDLAWRCCPGFTGKRCPEHLTDHGAASPQLEPEPQI |
| | | PSGQLDPGPRPPSYSRAAPSPHGRKG |
| | ERICH6 | FPDVRPRLASIVSPSLTSTFVPSQSATSTETPSASPPSS |
| | | TSSHKSFPKIFQTFRKDMSEMSI |
| 1852 | HHIPL2 | FAEDEAGELYFLATSYPSAYAPRGSIYKFVDPSRRAP |
| | | PGKCKYKPVPVRTKSKRIPFRPLAK |
| | B4GALNT1 | LACASLGLLYASTRDAPGLRLPLAPWAPPQSPRRPE |
| | | LPDLAPEPRYAHIPVRIKEQVVGLLA |
| | ABRA | ANENSIRQAQEPTGWLPGGTQDSPQAPKPITPPTSHQ |
| | | KAQSAPKSPPRLPEGHGDGQSSEKA |
| 1853 | EFS | HPLTRVAPQPPGEDDAPYDVPLTPKPPAELEPDLEW |
| | | EGGREPGPPIYAAPSNLKRASALLNL |
| 1854 | AEBP1 | PPPSRRRRPERVWPEPPEEKAPAPAPEERIEPPVKPLL |
| | | PPLPPDYGDGYVIPNYDDMDYYFG |
| | PLCH2 | TGSKGVADDVVPPGPGPAPEAPAQEGPGSGSPRDTR |
| | | PLSTQRPLPPLCSLETIAEEPAPGPG |
| | STAC2_0 | LKCPTEVLLTPPTPLPPPSPPPTASDRGLATPSPSPCP |
| | | VPRPLAALKPVRLHSFQEHVFKRA |
| | STAC2_1 | IRSSEEGPGDSASPVFTAPAESEGPGPEEKSPGQQLP |
| | | KATLRKDVGPMYSYVALYKFLPQEN |
| | MAPK8IP2_0 | EEEEEEEGDGEGQEGGDPGSEAPAPGPLIPSPSVEEP |
| | | HKHRPTTLRLTTLGAQDSLNNNGGF |
| 1855 | MAPK8IP2_1 | EEGDGEGQEGGDPGSEAPAPGPLIPSPSVEEPHKHRP |
| | | TTLRLTTLGAQDSLNNNGGFDLVRP |
| | PARMI | TNHSSTVTSTQPTGAPTAPESPTEESSSDHTPTSHAT |
| | | AEPVPQEKTPPTTVSGKVMCELIDM |
| | MMP28 | QSLYGKPLGGSVAVQLPGKLFTDFETWDSYSPQGR |
| | | RPETQGPKYCHSSFDAITVDRQQQLYI |
| 1856 | PRAC2 | NLLAFFLGLSGAGPIHLPMPWPNGRRHRVLDPHTQL |
| | | STHEAPGRWKPVAPRTMKACPQVLLE |
| | SPEF2 | EGKGKKGETALKRKGSPKGKSSGGKVPVKKSPADS |
| | | TDTSPVAIVPQPPKPGSEEWVYVNEPV |
| 1857 | CMYA5_0 | EAASPGLAASTQDGLDPDQEQPDLTSIERAEPVSAK |
| | | LTPTHPSVKGEKEENMLEPSISLSEP |
| 1858 | CMYA5_1 | ISELSSLLREESQNEEIKPFSPKIISLESKEPPASVAEG |
| | | GNPEEFQPFTFSLKGLSEEVSHP |
| | CMYA5_2 | EGKKPSPEVKIPTQRKPISSIHAREPQSPESPEVTQNP |
| | | PTQPKVAKPDLPEEKGKKGISSFK |
| 1859 | VOPP1 | FWFLLMMGVLFCCGAGFFIRRRMYPPPLIEEPAFNV |
| | | SYTRQPPNPGPGAQQPGPPYYTDPGG |
| | VPS37C_0 | PVRPVPQGTPPVVEEQPQPPLAMPPYPLPYSPSPSLP |
| | | VGPTAHGALPPAPFPVVSQPSFYSG |
| | VPS37C_1 | RPVPQGTPPVVEEQPQPPLAMPPYPLPYSPSPSLPVG |
| | | PTAHGALPPAPFPVVSQPSFYSGPL |
| 1860 | TNFRSF10D | WGQSVPTASSARAGRYPGARTASGTRPWLLDPKIL |
| | | KFVVFIVAVLLPVRVDSATIPRQDEVP |
| 1861 | DSC3 | NDNPPEILQEYVVICKPKMGYTDILAVDPDEPVHGA |
| | | PFYFSLPNTSPEISRLWSLTKVNDTA |
| | TMEM200B_0 | LRQGVLRAQALRPPDGPGWDCALLPSPGPRSPRAV |
| | | GCAEPEIWDPSPRRGTSPVPSVRSLRS |
| 1862 | TMEM200B_1 | QALRPPDGPGWDCALLPSPGPRSPRAVGCAEPEIWD |
| | | PSPRRGTSPVPSVRSLRSEPANPRLG |
| 1863 | INSRR | DGDLYLNDYCHRGLRLPTSNNDPRFDGEDGDPEAE |
| | | MESDCCPCQHPPPGQVLPPLEAQEASF |
| | PAPPA_0 | PCSPSGHWSPREAEGHPDVEQPCKSSVRTWSPNSAV |
| | | NPHTVPPACPEPQGCYLELEFLYPLV |
| 1864 | PAPPA_1 | PDVEQPCKSSVRTWSPNSAVNPHTVPPACPEPQGCY |
| | | LELEFLYPLVPESLTIWVTFVSTDWD |
| 1865 | HIVEP3_0 | SSGSHSSSHERCSLSQSSTAQSLEDPPPFVEPSSEHPL |
| | | SHKPEDTHTIKQKLALRLSERKKV |
| 1866 | HIVEP3_1 | AFESTKSQFGSPGPSDAARNLPLESTKSPAEPSKSVP |
| | | SLEGPTGFQPRTPKPGSGSESGKER |
| | HIVEP3_2 | GKGPGQDRPPLGPTVPYTEALQVFHHPVAQTPLHE |
| | | KPYLPPPVSLFSFQHLVQHEPGQSPEF |
| | HIVEP3_3 | SLFSFQHLVQHEPGQSPEFFSTQAMSSLLSSPYSMPP |
| | | LPPSLFQAPPLPLQPTVLHPGQLHL |
| | HIVEP3_4 | DYPKERERTGGGPGRPPDWTPHGTGAPAEPTPTHSP |
| | | CTPPDTLPRPPQGRRAAQSWSPRLES |
| | SEC31B_0 | TLHSKETSSYRLGSQPSHQVPTPSPRPRVFTPQSSPA |
| | | MPLAPSHPSPYQGPRTQNISDYRAP |
| | SEC31B_1 | PSHQVPTPSPRPRVFTPQSSPAMPLAPSHPSPYQGPR |
| | | TQNISDYRAPGPQAIQPLPLSPGVR |
| | NYAP1 | PQQPHALPPHAHRRPASALPSRRDGTPTKTTPCEIPP |
| | | PFPNLLQHRPPLLAFPQAKSASRTP |
| | CAMTA2_0 | AGGRRGNCFFIQDDDSGEELKGHGAAPPIPSPPPSPP |
| | | PSPAPLEPSSRVGRGEALFGGPVGA |
| 1867 | CAMTA2_1 | PDSLGRLPLSVAHSRGHVRLARCLEELQRQEPSVEP |
| | | PFALSPPSSSPDTGLSSVSSPSELSD |
| | CAMTA2_2 | VAHSRGHVRLARCLEELQRQEPSVEPPFALSPPSSSP |
| | | DTGLSSVSSPSELSDGTFSVTSAYS |
| | CAMTA2_3 | GHVRLARCLEELQRQEPSVEPPFALSPPSSSPDTGLS |
| | | SVSSPSELSDGTFSVTSAYSSAPDG |
| | SYNPO2L_0 | AYYGETDSDADGPATQEKPRRPRRRGPTRPTPPGAP |
| | | PDEVYLSDSPAEPAPTIPGPPSQGDS |
| | SYNPO2L_1 | TQEKPRRPRRRGPTRPTPPGAPPDEVYLSDSPAEPAP |
| | | TIPGPPSQGDSRVSSPSWEDGAALQ |
| | SYNPO2L_2 | GEGLQSPPRAQSAPPEAAVLPPSPLPAPVASPRPFQP |
| | | GGGAPTPAPSIFNRSARPFTPGLQG |
| | SYNPO2L_3 | ACNFMQPVGARSYKTLPHVTPKTPPPMAPKTPPPM |
| | | TPKTPPPVAPKPPSRGLLDGLVNGAAS |
| | SYNPO2L_4 | QPVGARSYKTLPHVTPKTPPPMAPKTPPPMTPKTPP |
| | | PVAPKPPSRGLLDGLVNGAASSAGIP |
| | SYNPO2L_5 | FAKRQSRADRYVVEGTPGPGLGPRPRSPSPTPSLPPS |
| | | WKYSPNIRAPPPIAYNPLLSPFFPQ |
| | MUC5B_0 | CCEYVPCGPSPAPGTSPQPSLSASTEPAVPTPTQTTA |
| | | TEKTTLWVTPSIRSTAALTSQTGSS |
| | MUC5B_1 | TPGTAHTTKVPTTTTTGFTATPSSSPGTALTPPVWIS |
| | | TTTTPTTTTPTTSGSTVTPSSIPGT |
| | MUC5B_2 | ASCKDMAKTWLVPDSRKDGCWAPTGTPPTASPAAP |
| | | VSSTPTPTPCPPQPLCDLMLSQVFAEC |
| | MUC5B_3 | LVPDSRKDGCWAPTGTPPTASPAAPVSSTPTPTPCPP |
| | | QPLCDLMLSQVFAECHNLVPPGPFF |
| | SCML4 | KIPKKRGRKPGYKIKSRVLMTPLALSPPRSTPEPDLS |
| | | SIPQDAATVPSLAAPQALTVCLYIN |
| | RIN3 | PPVLPLQPCSPAQPPVLPALAPAPACPLPTSPPVPAPH |
| | | VTPHAPGPPDHPNQPPMMTCERLP |
| | RBBP8NL | QRISNQLHGTIAVVRPGSQACPADRGPANGTPPPLP |
| | | ARSSPPSPAYERGLSLDSFLRASRPS |
| | ADGRG2_0 | VPKATSFAEPPDYSPVTHNVPSPIGEIQPLSPQPSAPI |
| | | ASSPAIDMPPQSETISSPMPQTHV |
| | ADGRG2_1 | PDYSPVTHNVPSPIGEIQPLSPQPSAPIASSPAIDMPPQ |
| | | SETISSPMPQTHVSGTPPPVKAS |
| | ADGRG2_2 | SAPIASSPAIDMPPQSETISSPMPQTHVSGTPPPVKAS |
| | | FSSPTVSAPANVNTTSAPPVQTDI |
| | ADGRG2_3 | DMPPQSETISSPMPQTHVSGTPPPVKASFSSPTVSAP |
| | | ANVNTTSAPPVQTDIVNTSSISDLE |
| 1868 | FAM193B | NGLVRRLNTVPNLSRVIWVKTPKPGYPSSEEPSSKE |
| | | VPSCKQELPEPVSSGGKPQKGKRQGS |
| 1869 | ZSCAN25 | RGAWEPGIQLGPVEVKPEWGMPPGEGVQGPDPGTE |
| | | EQLSQDPGDETRAFQEQALPVLQAGPG |
| 1870 | C9orf131_0 | QSPGTSPLEVLPGYETHLETTGHKKMPQAFEPPMPP |
| | | PCQSPASLSEPRKVSPEGGLAISKDF |
| 1871 | C9orf131_1 | THLETTGHKKMPQAFEPPMPPPCQSPASLSEPRKVS |
| | | PEGGLAISKDFWGTVGYREKPQASES |
| | C9orf131_2 | SSLSTPLPEPHIDLELVWRNVQQREVPQGPSPLAVDP |
| | | LHPVPQPPTLAEAVKIERTHPGLPK |
| 1872 | C9orf131_3 | PLPEPHIDLELVWRNVQQREVPQGPSPLAVDPLHPV |
| | | PQPPTLAEAVKIERTHPGLPKGVTCP |
| | SLC30A6 | VAANVLNFSDHHVIPMPLLKGTDDLNPVTSTPAKPS |
| | | SPPPEFSFNTPGKNVNPVILLNTQTR |
| | HEYL | FFHSCPGLPALSNQLAILGRVPSPVLPGVSSPAYPIPA |
| | | LRTAPLRRATGIILPARRNVLPSR |
| | SPPL2B | WTGSGFAKVLPPSPWAPAPADGPQPPKDSATPLSPQ |
| | | PPSEEPATSPWPAEQSPKSRTSEEMG |
| 1873 | DQX1 | SDSLQGLLQDARLEKLPGDLRVVVVTDPALEPKLR |
| | | AFWGNPPIVHIPREPGERPSPIYWDTI |
| | CACNB1 | EAERQALAQLEKAKTKPVAFAVRTNVGYNPSPGDE |
| | | VPVQGVAITFEPKDFLHIKEKYNNDWW |
| 1874 | COL25A1 | IKGEPGESGRPGQKGEPGLPGLPGLPGIKGEPGFIGP |
| | | QGEPGLPGLPGTKGERGEAGPPGRG |
| | PRR16 | YNIKNREVHLHSEPVHPPGKIPHQGPPLPPTPHLPPFP |
| | | LENGGMGISHSNSFPPIRPATVPP |
| 1875 | SYCP2L | TNMVEFMSAEDDRCLITLHLNDQSEPPVIGEPASDS |
| | | HLQPVPPFGVPDFPQQPKSHYRKHLF |
| 1876 | ASIC3 | QTFVSCQQQQLSFLPPPWGDCSSASLNPNYEPEPSDP |
| | | LGSPSPSPSPPYTLMGCRLACETRY |
| | TRABD2B | HTPAGQAIHSPAPQSPAPSPEGTSTSPAPVTPAAAVP |
| | | EAPSVTPTAPPEDEDPALSPHLLLP |
| | PRR18 | SSWPSATLKRPPARRGPGLDRTQPPAPPGVSPQALPS |
| | | RARAPATCAPPRPAGSGHSPARTTY |
| | UBALD1 | ATSSSAASSWPTAASPPGGPQHHQPQPPLWTPTPPSP |
| | | ASDWPPLAPQQATSEPRAHPAMEAE |
| 1877 | COL28A1 | PYGPKGPRGIQGITGPPGDPGPKGFQGNKGEPGPPGP |
| | | YGSPGAPGIGQQGIKGERGQEGRPG |
| | RTL3_0 | YDLLRKSSEAKEPQKLPEHMNPPAAWEAQKTPEFK |
| | | EPQKPPEPQDLLPWEPPAAWELQEAPA |
| 1878 | RTL3_1 | KSSEAKEPQKLPEHMNPPAAWEAQKTPEFKEPQKPP |
| | | EPQDLLPWEPPAAWELQEAPAAPESL |
| 1879 | OIT3 | PFLLLTCLFITGTSVSPVALDPCSAYISLNEPWRNTD |
| | | HQLDESQGPPLCDNHVNGEWYHFTG |
| | RNF149_0 | EMPAPESPPGRDPAANLSLALPDDDGSDDSSPPSASP |
| | | AESEPQCDPSFKGDAGENTALLEAG |
| | RNF149_1 | ESPPGRDPAANLSLALPDDDGSDDSSPPSASPAESEP |
| | | QCDPSFKGDAGENTALLEAGRSDSR |
| | PTPRQ | GYGNASNWISTKTLPGPPDGPPENVHVVATSPFSISI |
| | | SWSEPAVITGPTCYLIDVKSVDNDE |
| | PLSCR3 | YPEPALHPGPGQAPVPAQVPAPAPGFALFPSPGPVA |
| | | LGSAAPFLPLPGVPSGLEFLVQIDQI |
| 1880 | HAVCR1_0 | TTSIPTTTSVPVTTTVSTFVPPMPLPRQNHEPVATSPS |
| | | SPQPAETHPTTLQGAIRREPTSSP |
| | HAVCR1_1 | TTTSVPVTTTVSTFVPPMPLPRQNHEPVATSPSSPQP |
| | | AETHPTTLQGAIRREPTSSPLYSYT |
| 1880 | KRTAP2-4 | TCQTTVCRPVTCVPRCTRPICEPCRRPVCCDPCSLQE |
| | | GCCRPITCCPSSCTAVVCRPCCWAT |
| | DNAJC30 | RRKYDRGLLSDEDLRGPGVRPSRTPAPDPGSPRTPPP |
| | | TSRTHDGSRASPGANRTMFNFDAFY |
| | LPO | RKPALGAANRALARWLPAEYEDGLSLPFGWTPGKT |
| | | RNGFPLPLAREVSNKIVGYLNEEGVLD |
| | PYGO1 | SSNPYLGPGYPGFGGYSTFRMPPHVPPRMSSPYCGP |
| | | YSLRNQPHPFPQNPLGMGFNRPHAFN |
| | ADGRG4 | NYATSLNTPVSYPPWTPSSATLPSLTSFVYSPHSTEA |
| | | EISTPKTSPPPTSQMVEFPVLGTRM |
| | SYN3 | PGSSLFSSLSSAMKQAPQATSGLMEPPGPSTPIVQRP |
| | | RILLVIDDAHTDWSKYFHGKKVNGE |
| | MAP3K13 | SGMQTKRPDLLRSEGIPTTEVAPTASPLSGSPKMSTS |
| | | SSKSRYRSKPRHRRGNSRGSHSDFA |
| 1882 | TUT1 | FLDLGDLEEPQPVPKAPESPSLDSALASPLDPQALAC |
| | | TPASPPDSQPPASPQDSEALDFETP |
| | SFTPA2 | PGSHGLPGRDGRDGVKGDPGPPGPMGPPGETPCPPG |
| | | NNGLPGAPGVPGERGEKGEAGERGPP |
| 1883 | HECW1_0 | STLKDSSEKDGLSEVDTVAADPSALEEDREEPEGAT |
| | | PGTAHPGHSGGHFPSLANGAAQDGDT |
| | HECW1_1 | SSEKDGLSEVDTVAADPSALEEDREEPEGATPGTAH |
| | | PGHSGGHFPSLANGAAQDGDTHPSTG |
| | CELF3 | ITPSSGTSTPPAIAATPVSAIPAALGVNGYSPVPTQPT |
| | | GQPAPDALYPNGVHPYPAQSPAAP |
| 1884 | CCDC17_0 | ALQMQRGRAPLGPQDLRLLGDASLQPKGRRDPPLL |
| | | PPPVAPPLPPLPGFSEPQLPGTMTRNL |
| 1885 | CCDC17_1 | DASLQPKGRRDPPLLPPPVAPPLPPLPGFSEPQLPGT |
| | | MTRNLGLDSHFLLPTSDMLGPAPYD |
| | INAFM1 | AAVLLAVYYGLIWVPTRSPAAPAGPQPSAPSPPCAA |
| | | RPGVPPVPAPAAASLSCLLGVPGGPR |
| 1886 | GGN | EQIHSAPGPRRPAPALLAPPTFIFPAPTNGEPMRPGPP |
| | | GLQELPPLPPPTPPPTLQPPALQP |
| 1887 | CDX1_0 | SLGLGPQAYGPPAPPPAPPQYPDFSSYSHVEPAPAPP |
| | | TAWGAPFPAPKDDWAAAYGPGPAAP |
| | CDX1_1 | KDDWAAAYGPGPAAPAASPASLAFGPPPDFSPVPAP |
| | | PGPGPGLLAQPLGGPGTPSSPGAQRP |
| | TEX13D | SRSHSQGEGSERSQRMPLPGDSGCHNPLSESPQGTA |
| | | PLGSSGCHSQEEGTEGPQGMDPLGNR |
| 1888 | BEST2_0 | VSEASTGASCSCAVVPEGAAPECSCGDPLLDPGLPE |
| | | PEAPPPAGPEPLTLIPGPVEPFSIVT |
| 1889 | BEST2_1 | TGASCSCAVVPEGAAPECSCGDPLLDPGLPEPEAPPP |
| | | AGPEPLTLIPGPVEPFSIVTMPGPR |
| 1890 | BEST2_2 | PEGAAPECSCGDPLLDPGLPEPEAPPPAGPEPLTLIPG |
| | | PVEPFSIVTMPGPRGPAPPWLPSP |
| | NDST2 | FLQCWTRLRLQTLPPVPLAQKYFELFPQERSPLWQN |
| | | PCDDKRHKDIWSKEKTCDRLPKFLIV |
| 1891 | TNXB | VRTLCSLHGVFDLSRCTCSCEPGWGGPTCSDPTDAE |
| | | IPPSSPPSASGSCPDDCNDQGRCVRG |
| | SPATA31E1 | DPLGDVCKPVPAKAHQPHGKCMQDPSPASLSPPAPP |
| | | APLASTLSPGPMTFSEPFGPHSTLSA |
| 1892 | HTR3C | CTSPGRCCPTAPQKGNKGLGLTLTHLPGPKEPGELA |
| | | GKKLGPRETEPDGGSGWTKTQLMELW |
| 1893 | ITGAL | EGPITHQWSVQMEPPVPCHYEDLERLPDAAEPCLPG |
| | | ALFRCPVVFRQEILVQVIGTLELVGE |
| | SPATC1 | LAPQVATSYTPSSTTHIAQGAPHPPSRMHNSPTQNLP |
| | | VPHCPPHNAHSPPRTSSSPASVNDS |
| | SIGLEC12_0 | SARPAVGVGDTGMEDANAVRGSASQGPLIESPADD |
| | | SPPHHAPPALATPSPEEGEIQYASLSF |
| | SIGLEC12_1 | VGVGDTGMEDANAVRGSASQGPLIESPADDSPPHH |
| | | APPALATPSPEEGEIQYASLSFHKARP |
| 1894 | SOWAHA_0 | KQFVNNVAVVKELDGVKFVVLRKKPRPPEPEPAPF |
| | | GPPGAAAQPSKPTSTVLPRSASAPGAP |
| 1895 | SOWAHA_1 | AQPSKPTSTVLPRSASAPGAPPLVRVPRPVEPPGDLG |
| | | LPTEPQDTPGGPASEPAQPPGERSA |
| 1896 | SOWAHA_2 | LPRSASAPGAPPLVRVPRPVEPPGDLGLPTEPQDTPG |
| | | GPASEPAQPPGERSADPPLPALELA |
| 1897 | SOWAHA_3 | ALELAQATERPSADAAPPPRAPSEAASPCSDPPDAEP |
| | | GPGAAKGPPQQKPCMLPVRCVPAPA |
| | SOWAHA_4 | SVEESGLGLGLGPGRSPHLRRLSRAGPRLLSPDAEEL |
| | | PAAPPPSAVPLEPSEHEWLVRTAGG |
| | RAPGEF5_0 | VGSVKMQPPCESPALAAAAAVVAADGPLRRSPSAR |
| | | EPEREQPPASLRPRLRDLPALLRSGLT |
| 1898 | RAPGEF5_1 | MQPPCESPALAAAAAVVAADGPLRRSPSAREPERE |
| | | QPPASLRPRLRDLPALLRSGLTLRRKR |
| 1899 | PNRC1 | RLAPLGFSSRGYFGALPMVTTAPPPLPRIPDPRALPP |
| | | TLFLPHFLGGDGPCLTPQPRAPAAL |
| 1900 | THEG | PRGLQSSVYESRRVTDPERQDLDNAELGPEDPEEEL |
| | | PPEEVAGEEFPETLDPKEALSELERV |
| 1901 | PRSS36 | ARHLLLPLVMLVISPIPGAFQDSALSPTQEEPEDLDC |
| | | GRPEPSARIVGGSNAQPGTWPWQVS |
| | ADRB1 | RVFREAQKQVKKIDSCERRFLGGPARPPSPSPSPVPA |
| | | PAPPPGPPRPAAAAATAPLANGRAG |
| | CNGB1 | ATGAASDPAPPGRPQEMGPKLQARETPSLPTPIPLQP |
| | | KEEPKEAPAPEPQPGSQAQTSSLPP |
| 1902 | PROB1_0 | GIPRQLPTAPARRQDSSGSSGSYYTAPGSPEPPDVGP |
| | | DAKGPANWPWVAPGRGAGAQPRLSV |
| | PROB1_1 | DRTVQRARSPPFECRIPSEVPSRAVRPRSPSPPRQTPN |
| | | GAVRGPRCPSPQNLSPWDRTTRRV |
| | PROB1_2 | RARSPPFECRIPSEVPSRAVRPRSPSPPRQTPNGAVR |
| | | GPRCPSPQNLSPWDRTTRRVSSPLF |
| | PROB1_3 | QAPLPREPLALAGRTAPAQPRAASAPPTDRSPQSPSQ |
| | | GARRQPGAAPLGKVLVDPESGRYYF |
| | SPATA31D1 | LADLFSPSPLRDPLPPQPVSPLDSKFPIDHSPPQQLPF |
| | | PLLPPHHIERVESSLQPEASLSLN |
| | ARHGEF18 | RSLSPILPGRHSPAPPPDPGFPAPSPPPADSPSEGFSLK |
| | | AGGTALLPGPPAPSPLPATPLSA |
| 1903 | IL12RB2 | DLPTHDGYLPSNIDDLPSHEAPLADSLEELEPQHISLS |
| | | VFPSSSLHPLTFSCGDKLTLDQLK |
| | ALPK1 | SLQEPNNDNLEPSQNQPQQQMPLTPFSPHNTPGIFLA |
| | | PGAGLLEGAPEGIQEVRNMGPRNTS |
| | PRICKLE1 | EYAWVPPGLRPEQIQLYFACLPEEKVPYVNSPGEKH |
| | | RIKQLLYQLPPHDNEVRYCQSLSEEE |
| 1904 | B4GALNT3_0 | SKRNSTASFPGRTSHIPVQQPEKRKQKPSPEPSQDSP |
| | | HSDKWPPGHPVKNLPQMRGPRPRPA |
| | B4GALNT3_1 | TASFPGRTSHIPVQQPEKRKQKPSPEPSQDSPHSDKW |
| | | PPGHPVKNLPQMRGPRPRPAGDSPR |
| | KRTAP10-2 | QVDDCPESCCELPCGTPSCCAPAPCLTLVCTPVSCVS |
| | | SPCCQAACEPSACQSGCTSSCTPSC |
| | PRDM12 | CQSAYSQLAGLRAHQKSARHRPPSTALQAHSPALP |
| | | APHAHAPALAAAAAAAAAAAAHHLPAM |
| 1905 | USP9Y | RSHSARMTLAKACELCPEEEPDDQDAPDEHEPSPSE |
| | | DAPLYPHSPASQYQQNNHVHGQPYTG |
| 1906 | KRTAP2-3 | TCQTTVCRPVTCVPRCTRPICEPCRRPVCCDPCSLQE |
| | | GCCRPITCCPSSCTAVVCRPCCWAT |
| | POU6F2_0 | ELRGEDKAATSDSELNEPLLAPVESNDSEDTPSKLF |
| | | GARGNPALSDPGTPDQHQASQTHPPF |
| | POU6F2_1 | QQQQPPPSTNQHPQPAPQAPSQSQQQPLQPTPPQQP |
| | | PPASQQPPAPTSQLQQAPQPQQHQPH |
| | POU6F2_2 | QQHQPHSHSQNQNQPSPTQQSSSPPQKPSQSPGHGL |
| | | PSPLTPPNPLQLVNNPLASQAAAAAA |
| | POU6F2_3 | NQNQPSPTQQSSSPPQKPSQSPGHGLPSPLTPPNPLQ |
| | | LVNNPLASQAAAAAAAMSSIASSQA |
| 1907 | DSCAML1 | ASTATLPQRTLAMPAPPAGTAPPAPGPTPAEPPTAPS |
| | | AAPPAPSTEPPRAGGPHTKMGGSRD |
| | LDB3_0 | KIKSASYNLSLTLQKSKRPIPISTTAPPVQTPLPVIPHQ |
| | | KDPALDTNGSLVAPSPSPEARAS |
| 1908 | LDB3_1 | LTLQKSKRPIPISTTAPPVQTPLPVIPHQKDPALDTNG |
| | | SLVAPSPSPEARASPGTPGTPELR |
| | LDB3_2 | AAPAPKPRVVTTASIRPSVYQPVPASTYSPSPGANYS |
| | | PTPYTPSPAPAYTPSPAPAYTPSPV |
| | LDB3_3 | VVTTASIRPSVYQPVPASTYSPSPGANYSPTPYTPSP |
| | | APAYTPSPAPAYTPSPVPTYTPSPA |
| | LDB3_4 | SIRPSVYQPVPASTYSPSPGANYSPTPYTPSPAPAYTP |
| | | SPAPAYTPSPVPTYTPSPAPAYTP |
| | LDB3_5 | PVPASTYSPSPGANYSPTPYTPSPAPAYTPSPAPAYT |
| | | PSPVPTYTPSPAPAYTPSPAPNYNP |
| 1909 | SPAG4 | PRSHNWQTACGAATVRGGASEPTGSPVVSEEPLDL |
| | | LPTLDLRQEMPPPRVFKSFLSLLFQGL |
| 1910 | KIAA1549L_0 | KHPPRSDIPPLLPLPPSSSLAPDSPHSIISEPAEQSPKV |
| | | LLVPQTAPADPSLGQNIANPLIP |
| | KIAA1549L_1 | SDIPPLLPLPPSSSLAPDSPHSIISEPAEQSPKVLLVPQ |
| | | TAPADPSLGQNIANPLIPFSDEM |
| 1911 | IHO1 | IPIQTCKFNSKYQSPQPAISVPQSPFLGQQEPRAQPLH |
| | | LQCPRSPRKPVCPILGGTVMPNKT |
| 1912 | TTLL8 | KVELPACPCRHVDSQAPNTGVPVAQPAKSWDPNQL |
| | | NAHPLEPVLRGLKTAEGALRPPPGGKG |
| 1913 | TTLL3 | ELGPGRRGSASWYRQEGGAVCNWLRKPQPLEPRTS |
| | | FPSARRSEFRPPRRLPWAGPASAQSEE |
| 1914 | GGT6 | TSDLAGDALLSLLAGDLGVEVPSAVPRPTLEPAEQL |
| | | PVPQGILFTTPSPSAGPELLALLEAA |
| | FXYD5 | MDIQVPTRAPDAVYTELQPTSPTPTWPADETPQPQT |
| | | QTQQLEGTDGPLVTDPETHKSTKAAH |
| 1915 | DENND3 | GKTRMRSLRKKREKPRPEQWKGLPGPPRAPEPEDV |
| | | AVPGGVDLLTLPQLCFPGGVCVATEPK |
| | HGFAC_0 | CTSEGSAHRKWCATTHNYDRDRAWGYCVEATPPP |
| | | GGPAALDPCASGPCLNGGSCSNTQDPQS |
| 1916 | HGFAC_1 | WCATTHNYDRDRAWGYCVEATPPPGGPAALDPCA |
| | | SGPCLNGGSCSNTQDPQSYHCSCPRAFT |
| | KCNH6_0 | KPMPQGHASYILEAPASNDLALVPIASETTSPGPRLP |
| | | QGFLPPAQTPSYGDLDDCSPKHRNS |
| | KCNH6_1 | ASNDLALVPIASETTSPGPRLPQGFLPPAQTPSYGDL |
| | | DDCSPKHRNSSPRMPHLAVATDKTL |
| | KCNH6_2 | ASETTSPGPRLPQGFLPPAQTPSYGDLDDCSPKHRNS |
| | | SPRMPHLAVATDKTLAPSSEQEQPE |
| | ADAM19_0 | GCGKKCNGHGVCNNNQNCHCLPGWAPPFCNTPGH |
| | | GGSIDSGPMPPESVGPVVAGVLVAILVL |
| | ADAM19_1 | PFRVSQNSGTGHANPTFKLQTPQGKRKVINTPEILRK |
| | | PSQPPPRPPPDYLRGGSPPAPLPAH |
| | ESYT3 | KKSPATIFLTVPGPHSPGPIKSPRPMKCPASPFAWPP |
| | | KRLAPSMSSLNSLASSCFDLADISL |
| | SHANK1_0 | RSGRGRKGPLVKQTKVEGEPQKGGGLPPAPSPTSPA |
| | | SPQPPPAVAAPSEKNSIPIPTIIIKA |
| | SHANK1_1 | RGRKGPLVKQTKVEGEPQKGGGLPPAPSPTSPASPQ |
| | | PPPAVAAPSEKNSIPIPTIIIKAPST |
| | SHANK1_2 | PTQPEPTGGGGGGGSSPSPAPAMSPVPPSPSPVPTPA |
| | | SPSGPATLDFTSQFGAALVGAARRE |
| | SHANK1_3 | PVTSGRGPPSEDGPGVPPPSPRRSVPPSPTSPRASEEN |
| | | GLPLLVLPPPAPSVDVEDGEFLFV |
| | SHANK1_4 | PSVDVEDGEFLFVEPLPPPLEFSNSFEKPESPLTPGPP |
| | | HPLPDTPAPATPLPPVPPPAVAAA |
| | SHANK1_5 | DVEDGEFLFVEPLPPPLEFSNSFEKPESPLTPGPPHPL |
| | | PDTPAPATPLPPVPPPAVAAAPPT |
| | SHANK1_6 | EPLPPPLEFSNSFEKPESPLTPGPPHPLPDTPAPATPLP |
| | | PVPPPAVAAAPPTLDSTASSLTS |
| | SHANK1_7 | PLEFSNSFEKPESPLTPGPPHPLPDTPAPATPLPPVPPP |
| | | AVAAAPPTLDSTASSLTSYDSEV |
| | EMID1 | VSELTERLKVLEAKMTMLTVIEQPVPPTPATPEDPA |
| | | PLWGPPPAQGSPGDGGLQDQVGAWGL |
| | MYOZ3_0 | ELHIFPASPGASLGGPEGAHPAAAPAGCVPSPSALAP |
| | | GYAEPLKGVPPEKFNHTAISKGYRC |
| 1917 | MYOZ3_1 | ASLGGPEGAHPAAAPAGCVPSPSALAPGYAEPLKG |
| | | VPPEKFNHTAISKGYRCPWQEFVSYRD |
| 1918 | ZDHHC1 | MRTFRHMRPEPPGQAGPAAVNAKHSRPASPDPTPG |
| | | RRDCAGPPVQVEWDRKKPLPWRSPLLL |
| | DAB1_0 | PTVAGQFPPAAFMPTQTVMPLPAAMFQGPLTPLAT |
| | | VPGTSDSTRSSPQTDKPRQKMGKETFK |
| | DAB1_1 | QTVMPLPAAMFQGPLTPLATVPGTSDSTRSSPQTDK |
| | | PRQKMGKETFKDFQMAQPPPVPSRKP |
| | DAB1_2 | YFNKVGVAQDTDDCDDFDISQLNLTPVTSTTPSTNS |
| | | PPTPAPRQSSPSKSSASHASDPTTDD |
| | DAB1_3 | GVAQDTDDCDDFDISQLNLTPVTSTTPSTNSPPTPAP |
| | | RQSSPSKSSASHASDPTTDDIFEEG |
| | DAB1_4 | DFDISQLNLTPVTSTTPSTNSPPTPAPRQSSPSKSSAS |
| | | HASDPTTDDIFEEGFESPSKSEEQ |
| 1919 | COL13A1 | LDGRPGPPGTPGPIGVPGPAGPKGERGSKGDPGMTG |
| | | PTGAAGLPGLHGPPGDKGNRGERGKK |
| | VEGFB | SAVKPDRAATPHHRPQPRSVPGWDSAPGAPSPADIT |
| | | HPTPAPGPSAHAAPSTTSALTPGPAA |
| | TOX2_0 | PSFPLSPTLHQQLSLPPHAQGALLSPPVSMSPAPQPP |
| | | VLPTPMALQVQLAMSPSPPGPQDFP |
| | TOX2_1 | QQLSLPPHAQGALLSPPVSMSPAPQPPVLPTPMALQ |
| | | VQLAMSPSPPGPQDFPHISEFPSSSG |
| | MAP3K12 | GLLKPHPSRGLLHGNTMEKLIKKRNVPQKLSPHSKR |
| | | PDILKTESLLPKLDAALSGVGLPGCP |
| 1920 | PARP6 | EVVDLLVAMCRAALESPRKSIIFEPYPSVVDPTDPKT |
| | | LAFNPKKKNYERLQKALDSVMSIRE |
| | NLGN1 | EILGPVIQFLGVPYAAPPTGERRFQPPEPPSPWSDIRN |
| | | ATQFAPVCPQNIIDGRLPEVMLPV |
| | POM121_0 | SSPAAPAASSAPPMFKPIFTAPPKSEKEGPTPPGPSVT |
| | | ATAPSSSSLPTTTSTTAPTFQPVF |
| | POM121_1 | AADFSGFGSTLATSAPATSSQPTLTFSNTSTPTFNIPF |
| | | GSSAKSPLPSYPGANPQPAFGAAE |
| 1921 | GPR137 | GCSWEHSRGESTRCQDQAATTTVSTPPHRRDPPPSP |
| | | TEYPGPSPPHPRPLCQVCLPLLAQDP |
| 1922 | PPFIA4 | SALREESAKDWETSPLPGMLAPAAGPAFDSDPEISD |
| | | VDEDEPGGLVGSADVVSPSGHSDAQT |
| | PCDH15_0 | LGPMFLPCVLVPNTRDCRPLTYQAAIPELRTPEELNP |
| | | IIVTPPIQAIDQDRNIQPPSDRPGI |
| | PCDH15_1 | VPNTRDCRPLTYQAAIPELRTPEELNPIIVTPPIQAID |
| | | QDRNIQPPSDRPGILYSILVGTPE |
| | PCDH15_2 | PISPPSPPPAPAPLAPPPDISPFSLFCPPPSPPSIPLPLPPP |
| | | TFFPLSVSTSGPPTPPLLPP |
| | COL4A6 | PCIIPGSYGPSGFPGTPGFPGPKGSRGLPGTPGQPGSS |
| | | GSKGEPGSPGLVHLPELPGFPGPR |
| 1923 | NT5C1B | LRKTDSRGYLVRSQWSRISRSPSTKAPSIDEPRSRNT |
| | | SAKLPSSSTSSRTPSTSPSLHDSSP |
| | MCIDAS_0 | SDSSSMMSPTLASGDFPFSPCDISPFGPCLSPPLDPRA |
| | | LQSPPLRPPDVPPPEQYWKEVADQ |
| | MCIDAS_1 | LASGDFPFSPCDISPFGPCLSPPLDPRALQSPPLRPPD |
| | | VPPPEQYWKEVADQNQRALGDALV |
| | NEUROD1 | PPYGTMDSSHVFHVKPPPHAYSAALEPFFESPLTDC |
| | | TSPSFDGPLSPPLSINGNFSFKHEPS |
| | SPATA31A5_0 | SLSASQPPEPSLPLEHPSPEPPALFPHPPHTPDPLACSL |
| | | PPPKGFTAPPLRDSTLITPSHCD |
| 1924 | SPATA31A5_1 | SASQPPEPSLPLEHPSPEPPALFPHPPHTPDPLACSLPP |
| | | PKGFTAPPLRDSTLITPSHCDSV |
| 1925 | ADAM33 | PKDGPHRDHPLGGVHPMELGPTATGQPWPLDPENS |
| | | HEPSSHPEKPLPAVSPDPQADQVQMPR |
| | GCM2 | LSSCNYAPEDTGMSVYPEPWGPPVTVTRAASPSGPP |
| | | PMKIAGDCRAIRPTVAIPHEPVSSRT |
| 1926 | PLCH1 | NRAKFKANGNCGYVLKPQQMCKGTFNPFSGDPLPA |
| | | NPKKQLILKVISGQQLPKPPDSMFGDR |
| 1927 | LAMA5 | LPPGLPLTHAQDLTPAMSPAGPRPRPPTAVDPDAEP |
| | | TLLREPQATVVFTTHVPTLGRYAFLL |
| 1928 | TTLL10 | QPGARRPAPPPLVPQRPRPPGPDLDSAHDGEPQAPG |
| | | TEQSGTGNRHPAQEPSPGTAKEEREE |
| 1929 | LONRF2 | RPEELEELAGGLVRAVGLRDRPLSAENPGGEPEAPG |
| | | EGGPAPEPRAPRDLLGCPRCRRLLHK |
| 1930 | MXRA7_0 | ASPEPARAPPEPAPPAEATGAPAPSRPCAPEPAASPA |
| | | GPEEPGEPAGLGELGEPAGPGEPEG |
| 1931 | MXRA7_1 | EPAPPAEATGAPAPSRPCAPEPAASPAGPEEPGEPAG |
| | | LGELGEPAGPGEPEGPGDPAAAPAE |
| | TOGARAM2 | PSPLPPGQGVLTGLRAPRTRLARGSGPREKTPASLEP |
| | | KPLASPIRDRPAAAKKPALPFSQSA |
| 1932 | KIF17 | RLSSTVARTDAPQADVPKVPVQVPAPTDLLEPSDAR |
| | | PEAEAADDFPPRPEVDLASEVALEVV |
| | COL4A3_0 | GSKGERGRPGKDAMGTPGSPGCAGSPGLPGSPGPPG |
| | | PPGDIVFRKGPPGDHGLPGYLGSPGI |
| | COL4A3_1 | GEPGLQGTQGVPGAPGPPGEAGPRGELSVSTPVPGP |
| | | PGPPGPPGHPGPQGPPGIPGSLGKCG |
| | COL4A3_2 | PHGDLGFKGIKGLLGPPGIRGPPGLPGFPGSPGPMGI |
| | | RGDQGRDGIPGPAGEKGETGLLRAP |
| | COL4A3_3 | DKGSMGHPGPKGPPGTAGDMGPPGRLGAPGTPGLP |
| | | GPRGDPGFQGFPGVKGEKGNPGFLGSI |
| 1933 | COL4A3_4 | VKGEKGNPGFLGSIGPPGPIGPKGPPGVRGDPGTLKI |
| | | ISLPGSPGPPGTPGEPGMQGEPGPP |
| | GRIN2C | GRRAPPPSPCPTPRSGPSPCLPTPDPPPEPSPTGWGPP |
| | | DGGRAALVRRAPQPPGRPPTPGPP |
| 1934 | LRRC37B_0 | VEVTMTSEPKNETESTQAQQEAPIQPPEEAEPSSTAL |
| | | RTTDPPPEHPEVTLPPSDKGQAQHS |
| 1935 | LRRC37B_1 | NETESTQAQQEAPIQPPEEAEPSSTALRTTDPPPEHPE |
| | | VTLPPSDKGQAQHSHLTEATVQPL |
| | SOHLH1 | DPGTGASSGTRTPDVKAFLESPWSLDPASASPEPVP |
| | | HILASSRQWDPASCTSLGTDKCEALL |
| | ZNF469_0 | QPAAEELGFHRCFQEPPSSFTSTNYTSPSATPRPPAP |
| | | GPPQSRGTSPLQPGSYPEYQASGAD |
| | ZNF469_1 | QGGSQGALGTAGKTPGPREKLPAVRSSQGGSPALFT |
| | | YNGMTDPGAQPLFFGVAQPQVSPHGT |
| 1936 | ZNF469_2 | ESQLPGPLGPSAFFHPPTHPQETGSPFPSPEPPHSLPT |
| | | HYQPEPAKAFPFPADGLGAEGAFQ |
| 1937 | ZNF469_3 | RGPSSGHPLKSKAGVTPESKAPPPLPAATPDPQTPRP |
| | | GDRGCPARGRPKTRSLGLAPTEADA |
| | ZNF469_4 | GDLAACAPSPTSAAHMPCSLGPLPREDPLTSPSRAQ |
| | | GGLGGQLPASPSCRDPPGPQQLLACS |
| 1938 | ZNF469_5 | LQGLPDNPDTQGGVQGPEGPTPDASGSSAKDPPSLF |
| | | DDEVSFSQLFPPGGRLTRKRNPHVYG |
| | ZNF469_6 | PGPARSESVGSFGRAPSAPDKPPRTPRKQATPSRVLP |
| | | TKPKPNSQNKPRPPPSEQRKAEPGH |
| 1939 | PWWP3A | SGVREDDPCANAEGHDPGLPLGSLTAPPAPEPSACS |
| | | EPGECPAKKRPRLDGSQRPPAVQLEP |
| 1940 | APC2_0 | IDKELLEAQDRVQQTEPQALLAVKSVPVDEDPETEV |
| | | PTHPEDGTPQPGNSKVEVVFWLLSML |
| 1941 | APC2_1 | APPPARTQPSLIADETPPCYSLSSSASSLSEPEPSEPPA |
| | | VHPRGREPAVTKDPGPGGGRDSS |
| 1942 | APC2_2 | RTQPSLIADETPPCYSLSSSASSLSEPEPSEPPAVHPR |
| | | GREPAVTKDPGPGGGRDSSPSPRA |
| 1943 | APC2_3 | TPPCYSLSSSASSLSEPEPSEPPAVHPRGREPAVTKDP |
| | | GPGGGRDSSPSPRAAEELLQRCIS |
| | CCDC80 | VTRSTSRAVTVAARPMTTTAFPTTQRPWTPSPSHRP |
| | | PTTTEVITARRPSVSENLYPPSRKDQ |
| 1944 | POU5F1B_0 | MAGHLASDFAFSPPPGGGGDGPWGAEPGWVDPLT |
| | | WLSFQGPPGGPGIGPGVGPGSEVWGIPP |
| | POU5F1B_1 | YAQREDFEAAGSPFSGGPVSFPPAPGPHFGTPGYGSP |
| | | HFTALYSSVPFPEGEVFPPVSVITL |
| | POU5F1B_2 | DFEAAGSPFSGGPVSFPPAPGPHFGTPGYGSPHFTAL |
| | | YSSVPFPEGEVFPPVSVITLGSPMH |
| | COL4A4_0 | GRKGESGIGAKGEKGIPGFPGPRGDPGSYGSPGFPGL |
| | | KGELGLVGDPGLFGLIGPKGDPGNR |
| 1945 | COL4A4_1 | PPGCPGDHGMPGLRGQPGEMGDPGPRGLQGDPGIP |
| | | GPPGIKGPSGSPGLNGLHGLKGQKGTK |
| 1946 | COL4A4_2 | PHGFPGPPGEKGLPGPPGRKGPTGLPGPRGEPGPPA |
| | | DVDDCPRIPGLPGAPGMRGPEGAMGL |
| | SULT1A4 | KCNRAPIYVRVPFLEVNDPGEPSGLETLKDTPPPRLI |
| | | KSHLPLALLPQTLLDQKVKVVYVAR |
| | SULT1A3 | KCNRAPIYVRVPFLEVNDPGEPSGLETLKDTPPPRLI |
| | | KSHLPLALLPQTLLDQKVKVVYVAR |
| | ADGRL1_0 | GPPDPSAGPATSPPLSTTTTARPTPLTSTASPAATTPL |
| | | RRAPLTTHPVGAINQLGPDLPPAT |
| | ADGRL1_1 | SAGPATSPPLSTTTTARPTPLTSTASPAATTPLRRAPL |
| | | TTHPVGAINQLGPDLPPATAPVPS |
| 1947 | ODF3 | HKTPGPAAYRQTDVRVTKFKAPQYTMAARVEPPG |
| | | DKTLKPGPGAHSPEKVTLTKPCAPVVTF |
| 1948 | COL1A2_0 | PMGLMGPRGPPGAAGAPGPQGFQGPAGEPGEPGQT |
| | | GPAGARGPAGPPGKAGEDGHPGKPGRP |
| | COL1A2_1 | ASGPAGVRGPNGDAGRPGEPGLMGPRGLPGSPGNI |
| | | GPAGKEGPVGLPGIDGRPGPIGPAGAR |
| | WIZ_0 | CLIKKEPPAGDLAPALAEDGPPTVAPGPVQSPLPLSP |
| | | LAGRPGKPGAGPAQVPRELSLTPIT |
| | WIZ_1 | EPPAGDLAPALAEDGPPTVAPGPVQSPLPLSPLAGRP |
| | | GKPGAGPAQVPRELSLTPITGAKPS |
| | CBLL2 | DHIQNNSDSGAKKPTPPDYYPECQSQPAVSSPHHIIP |
| | | QKQHYAPPPSPSSPVNHQMPYPPQD |
| | ATXN7_0 | SAVGPTCPATVSSLVKPGLNCPSIPKPTLPSPGQILNG |
| | | KGLPAPPTLEKKPEDNSNNRKFLN |
| | ATXN7_1 | KPHTPSLPRPPGCPAQQGGSAPIDPPPVHESPHPPLPA |
| | | TEPASRLSSEEGEGDDKEESVEKL |
| 1949 | CHRDL2_0 | YCLRCTCSEGAHVSCYRLHCPPVHCPQPVTEPQQCC |
| | | PKCVEPHTPSGLRAPPKSCQHNGTMY |
| 1950 | CHRDL2_1 | AHVSCYRLHCPPVHCPQPVTEPQQCCPKCVEPHTPS |
| | | GLRAPPKSCQHNGTMYQHGEIFSAHE |
| | FLRT2 | MAVRELNMNLLSCPTTTPGLPLFTPAPSTASPTTQPP |
| | | TLSIPNPSRSYTPPTPTTSKLPTIP |
| | GRB10_0 | VRRLQEEDQQFRTSSLPAIPNPFPELCGPGSPPVLTPG |
| | | SLPPSQAAAKQDVKVFSEDGTSKV |
| | GRB10_1 | EEDQQFRTSSLPAIPNPFPELCGPGSPPVLTPGSLPPS |
| | | QAAAKQDVKVFSEDGTSKVVEILA |
| | TNFRSF10C_0 | CTSWDDIQCVEEFGANATVETPAAEETMNTSPGTPA |
| | | PAAEETMNTSPGTPAPAAEETMTTSP |
| | TNFRSF10C_1 | NATVETPAAEETMNTSPGTPAPAAEETMNTSPGTPA |
| | | PAAEETMTTSPGTPAPAAEETMTTSP |
| | TNFRSF10C_2 | SPGTPAPAAEETMNTSPGTPAPAAEETMTTSPGTPA |
| | | PAAEETMTTSPGTPAPAAEETMITSP |
| | TNFRSF10C_3 | SPGTPAPAAEETMTTSPGTPAPAAEETMTTSPGTPAP |
| | | AAEETMITSPGTPASSHYLSCTIVG |
| | PIK3C2B | SGKPVARSKTMPPQVPPRTYASRYGNRKNATPGKN |
| | | RRISAAPVGSRPHTVANGHELFEVSEE |
| | PRPF40B | AGKQQQQLPQTLQPQPPQPQPDPPPVPPGPTPVPTG |
| | | LLEPEPGGSEDCDVLEATQPLEQGFL |
| | OLFML2B | SVLQPSPQVPATTVAHTATQQPAAPAPPAVSPREAL |
| | | MEAMHTVPVPPTTVRTDSLGKDAPAG |
| | GRIN2D | RYYGPIEPQGLGLGLGEARAAPRGAAGRPLSPPAAQ |
| | | PPQKPPPSYFAIVRDKEPAEPPAGAF |
| 1951 | CISH | VASCTADTRSDSPDPAPTPALPMPKEDAPSDPALPA |
| | | PPPATAVHLKLVQPFVRRSSARSLQH |
| 1952 | RRBP1 | KKGKTKKKEEKPNGKIPDHDPAPNVTVLLREPVRA |
| | | PAVAVAPTPVQPPIIVAPVATVPAMPQ |
| | GFY | LLAGLRSKAAPSAPLPLGCGFPDMAHPSETSPLKGA |
| | | SENSKRDRLNPEFPGTPYPEPSKLPH |
| 1953 | FRMD7 | QVFFYVDKPPQVPRWSPIRAEERTSPHSYVEPTAMK |
| | | PAERSPRNIRMKSFQQDLQVLQEAIA |
| | TBXT | NHRWKYVNGEWVPGGKPEPQAPSCVYIHPDSPNFG |
| | | AHWMKAPVSFSKVKLTNKLNGGGQIML |
| 1954 | PLPPR3 | DLLAPRSPMAKENMVTFSHTLPRASAPSLDDPARRH |
| | | MTIHVPLDASRSKQLISEWKQKSLEG |
| 1955 | FREM1_0 | DYDRMASLECTVSLDTARTRLPAHGQMVLGEPRPE |
| | | EPRGDQPHSFFPESQLRAKLKCPGGSC |
| 1956 | FREM1_1 | ASLECTVSLDTARTRLPAHGQMVLGEPRPEEPRGDQ |
| | | PHSFFPESQLRAKLKCPGGSCTPGLK |
| 1957 | NAPSA | QGLLDKPVFSFYLNRDPEEPDGGELVLGGSDPAHYI |
| | | PPLTFVPVTVPAYWQIHMERVKVGPG |
| | ARHGAP44 | GTACAGTQPGAQPGAQPGASPSPSQPPADQSPHTLR |
| | | KVSKKLAPIPPKVPFGQPGAMADQSA |
| | ASCL2 | VRNALAGGLRPQAVRPSAPRGPPGTTPVAASPSRAS |
| | | SSPGRGGSSEPGSPRSAYSSDDSGCE |
| 1958 | PIP4P1 | PGGGLTPSAPPYGAAFPPFPEGHPAVLPGEDPPPYSP |
| | | LTSPDSGSAPMITCRVCQSLINVEG |
| 1959 | ASXL3 | KEKRARIEDDQSTRNISSSSPPEKEQPPREEPRVPPLK |
| | | IQLSKIGPPFIIKSQPVSKPESRA |
| | DOK3 | AIARQRERLPELTRPQPCPLPRATSLPSLDTPGELRE |
| | | MPPGPEPPTSRKMHLAEPGPQSLPL |
| 1960 | HS6ST3 | PEGPRGAAAPEEEDEEPGDPREGEEEEEEDEPDPEAP |
| | | ENGSLPRFVPRFNFSLKDLTRFVDF |
| | DLX5 | VFDRRVPSIRSGDFQAPFQTSAAMHHPSQESPTLPES |
| | | SATDSDYYSPTGGAPHGYCSPTSAS |
| | MAP3K14 | SLAHAGVALAKPLPRTPEQESCTIPVQEDESPLGAPY |
| | | VRNTPQFTKPLKEPGLGQLCFKQLG |
| 1961 | XAGE3 | WRGRSTYRPRPRRSVPPPELIGPMLEPGDEEPQQEEP |
| | | PTESRDPAPGQEREEDQGAAETQVP |
| | PAX9 | LAQQGHYDSYKQHQPTPQPALPYNHIYSYPSPITAA |
| | | AAKVPTPPGVPAIPGSVAMPRTWPSS |
| 1962 | ARHGEF15_0 | SRASLDSQTSPDSPSSTPTPSPVSRRSASPEPAPRSPV |
| | | PPPKPSGSPCTPLLPMAGVLAQNG |
| | ARHGEF15_1 | DSQTSPDSPSSTPTPSPVSRRSASPEPAPRSPVPPPKPS |
| | | GSPCTPLLPMAGVLAQNGSASAP |
| 1963 | NEDD9_0 | EGVYDIPPTCTKPAGKDLHVKYNCDIPGAAEPVARR |
| | | HQSLSPNHPPPQLGQSVGSQNDAYDV |
| | NEDD9_1 | TKPAGKDLHVKYNCDIPGAAEPVARRHQSLSPNHPP |
| | | PQLGQSVGSQNDAYDVPRGVQFLEPP |
| | MUC7_0 | NTSSSVATLAPVNSPAPQDTTAAPPTPSATTPAPPSS |
| | | SAPPETTAAPPTPSATTQAPPSSSA |
| | MUC7_1 | ETTAAPPTPSATTQAPPSSSAPPETTAAPPTPPATTPA |
| | | PPSSSAPPETTAAPPTPSATTPAP |
| | MUC7_2 | PPTPSATTQAPPSSSAPPETTAAPPTPPATTPAPPSSSA |
| | | PPETTAAPPTPSATTPAPLSSSA |
| | MUC7_3 | ETTAAPPTPPATTPAPPSSSAPPETTAAPPTPSATTPA |
| | | PLSSSAPPETTAVPPTPSATTLDP |
| | MUC7_4 | PPTPPATTPAPPSSSAPPETTAAPPTPSATTPAPLSSSA |
| | | PPETTAVPPTPSATTLDPSSASA |
| | MUC7_5 | PPTPSATTLDPSSASAPPETTAAPPTPSATTPAPPSSP |
| | | APQETTAAPITTPNSSPTTLAPDT |
| | RCAN2 | KLYFAQVQTPETDGDKLHLAPPQPAKQFLISPPSSPP |
| | | VGWQPINDATPVLNYDLLYAVAKLG |
| 1964 | RPH3AL | AWFYKGLPKYILPLKTPGRADDPHFRPLPTEPAERE |
| | | PRSSETSRIYTWARGRVVSSDSDSDS |
| | MXRA8 | HLHHHYCGLHERRVFHLTVAEPHAEPPPRGSPGNGS |
| | | SHSGAPGPDPTLARGHNVINVIVPES |
| | STON1_0 | EFPSGSSSTSSTPLSSPIVDFYFSPGPPSNSPLSTPTKD |
| | | FPGFPGIPKAGTHVLYPIPESSS |
| | STON1_1 | ISGGESSLLPTRPTCLSHALLPSDHSCTHPTPKVGLPD |
| | | EVNPQQAESLGFQSDDLPQFQYFR |
| | MYBPC1 | MPEPTKKEENEVPAPAPPPEEPSKEKEAGTTPAKDW |
| | | TLVETPPGEEQAKQNANSQLSILFIE |
| | SIMC1_0 | DVPGLPQSILHPQDVAYLQDMPRSPGDVPQSPSDVS |
| | | PSPDAPQSPGGMPHLPGDVLHSPGDM |
| | SIMC1_1 | PQSILHPQDVAYLQDMPRSPGDVPQSPSDVSPSPDA |
| | | PQSPGGMPHLPGDVLHSPGDMPHSSG |
| | SIMC1_2 | GDRPDFTQNDVQNRDMPMDISALSSPSCSPSPQSET |
| | | PLEKVPWLSVMETPARKEISLSEPAK |
| 1966 | KRTAP2-2 | TCQTTVCRPVTCVPRCTRPICEPCRRPVCCDPCSLQE |
| | | GCCRPITCCPSSCTAVVCRPCCWAT |
| | CHPF2_0 | FFPVHFQEFNPALSPQRSPPGPPGAGPDPPSPPGADPS |
| | | RGAPIGGRFDRQASAEGCFYNADY |
| 1967 | CHPF2_1 | FQEFNPALSPQRSPPGPPGAGPDPPSPPGADPSRGAPI |
| | | GGRFDRQASAEGCFYNADYLAARA |
| | SPATA22 | GCLPVPLFNQKKRNRQPLTSNPLKDDSGISTPSDNY |
| | | DFPPLPTDWAWEAVNPELAPVMKTVD |
| | TOGARAM1 | QNPSPGAYILPSYPVSSPRTSPKHTSPLIISPKKSQDNS |
| | | VNFSNSWPLKSFEGLSKPSPQKK |
| 1968 | HS3ST6 | ALVLGAYCLCALPGRCPPAARAPAPAPAPSEPSSSV |
| | | HRPGAPGLPLASGPGRRRFPQALIVG |
| | ZCWPW1 | QNKEECGKGPKRIFAPPAQKSYSLLPCSPNSPKEETP |
| | | GISSPETEARISLPKASLKKKEEKA |
| 1969 | TGFBR3L | LHTLTQPIVVTVPRPPPRPPKSVPGRAVRPEPPAPAP |
| | | AALEPAPVVALVLAAFVLGAALAAG |
| 1970 | EFCAB8 | SSLSPESVANTNLRRSLVSAPPVMRCPRDKEPDRPV |
| | | PQQKPSSASGTSRQSSKIHSKQSIYK |
| | LTBR_0 | TGGSMTITGNIYIYNGPVLGGPPGPGDLPATPEPPYPI |
| | | PEEGDPGPPGLSTPHQEDGKAWHL |
| 1971 | LTBR_1 | GSMTITGNIYIYNGPVLGGPPGPGDLPATPEPPYPIPE |
| | | EGDPGPPGLSTPHQEDGKAWHLAE |
| 1972 | LTBR_2 | IYNGPVLGGPPGPGDLPATPEPPYPIPEEGDPGPPGLS |
| | | TPHQEDGKAWHLAETEHCGATPSN |
| | TSPOAP1 | PPPCCCSIPQPCRGSGPKDLDLPPGSPGRCTPKSSEPA |
| | | PATLTGVPRRTAKKAESLSNSSHS |
| | NLRP1 | TSGRRWREISASLLYQALPSSPDHESPSQESPNAPTS |
| | | TAVLGSWGSPPQPSLAPREQEAPGT |
| | PLXND1_0 | VYLAAVNRLYQLSGANLSLEAEAAVGPVPDSPLCH |
| | | APQLPQASCEHPRRLTDNYNKILQLDP |
| | PLXND1_1 | LSAQWPCFWCSQQHSCVSNQSRCEASPNPTSPQDCP |
| | | RTLLSPLAPVPTGGSQNILVPLANTA |
| | PLXND1_2 | SQQHSCVSNQSRCEASPNPTSPQDCPRTLLSPLAPVP |
| | | TGGSQNILVPLANTAFFQGAALECS |
| | FLI1 | LSVVSDDQSLFDSAYGAAAHLPKADMTASGSPDYG |
| | | QPHKINPLPPQQEWINQPVRVNVKREY |
| 1973 | PANX2 | LSQAEDCGLGLAPAPIKDAPLPEKEIPYPTEPARAGL |
| | | PSGGPFHVRSPPAAPAVAPLTPASL |
| 1974 | CACNA1H | EGKGSTDDEAEDGRAAPGPRATPLRRAESLDPRPLR |
| | | PAALPPTKCRDRDGQVVALPSDFFLR |
| 1975 | COL7A1_0 | RPGSPGRAGNPGTPGAPGLKGSPGLPGPRGDPGERG |
| | | PRGPKGEPGAPGQVIGGEGPGLPGRK |
| 1976 | COL7A1_1 | QVIGGEGPGLPGRKGDPGPSGPPGPRGPLGDPGPRG |
| | | PPGLPGTAMKGDKGDRGERGPPGPGE |
| 1977 | COL7A1_2 | GPAGPRGATGVQGERGPPGLVLPGDPGPKGDPGDR |
| | | GPIGLTGRAGPPGDSGPPGEKGDPGRP |
| 1978 | COL7A1_3 | ERGEQGRDGPPGLPGTPGPPGPPGPKVSVDEPGPGL |
| | | SGEQGPPGLKGAKGEPGSNGDQGPKG |
| | COL7A1_4 | GPPGRGLTGPTGAVGLPGPPGPSGLVGPQGSPGLPG |
| | | QVGETGKPGAPGRDGASGKDGDRGSP |
| | COL7A1_5 | GEPGDPGEDGQKGAPGPKGFKGDPGVGVPGSPGPP |
| | | GPPGVKGDLGLPGLPGAPGVVGFPGQT |
| 1979 | CDH2 | RDNILKYDEEGGGEEDQDYDLSQLQQPDTVEPDAIK |
| | | PVGIRRMDERPIHAEPQYPVRSAAPH |
| 1980 | FBXO24 | RECLYILSSHDIEQHAPYRHLPASRVVGTPEPSLGAR |
| | | APQDPGGMAQACEEYLSQIHSCQTL |
| | USP30 | LLGHKPSQHNPKLNKNPGPTLELQDGPGAPTPVLNQ |
| | | PGAPKTQIFMNGACSPSLLPTLSAPM |
| | NPAPI | GLTSPSVQPLSGSIIPPGFAELTSPYTALGTPVNAEPV |
| | | EGHNASAFPNGTAKTSGFRIATGM |
| | RBMS3 | AASPVSTYQVQSTSWMPHPPYVMQPTGAVITPTMD |
| | | HPMSMQPANMMGPLTQQMNHLSLGTTG |
| 1981 | SAC3D1 | PAAERAQREREHRLHRLEVVPGCRQDPPRADPQRA |
| | | VKEYSRPAAGKPRPPPSQLRPPSVLLA |
| 1982 | SP7 | PAGSPPAPTSGYANDYPPFSHSFPGPTGTQDPGLLVP |
| | | KGHSSSDCLPSVYTSLDMTHPYGSW |
| | ANKLE1_0 | VPRSQGTEAELNARLQALTLTPPNAAGFQSSPSSMP |
| | | LLDRSPAHSPPRTPTPGASDCHCLWE |
| | ANKLE1_1 | LNARLQALTLTPPNAAGFQSSPSSMPLLDRSPAHSPP |
| | | RTPTPGASDCHCLWEHQTSIDSDMA |
| | MEF2B | SGGRSLGEEGPPTRGASPPTPPVSIKSERLSPAPGGPG |
| | | DFPKTFPYPLLLARSLAEPLRPGP |
| | VGLL2_0 | LAYYSKMQEAQECNASPSSSGSGSSSFSSQTPASIKE |
| | | EEGSPEKERPPEAEYINSRCVLFTY |
| 1983 | VGLL2_1 | MPAASGRPARLATAPAPAPGSPPCELSGKGEPAGAA |
| | | WAGPGGPFASPSGDVAQGLGLSVDSA |
| | ESRRB | RGSPKDERMSSHDGKCPFQSAAFTSRDQSNSPGIPN |
| | | PRPSSPTPLNERGRQISPSTRTPGGQ |
| | GALNT6 | RDSMPKLQIRAPEAQQTLFSINQSCLPGFYTPAELKP |
| | | FWERPPQDPNAPGADGKAFQKSKWT |
| | RBM38 | TYGLTPHYIYPPAIVQPSVVIPAAPVPSLSSPYIEYTP |
| | | ASPAYAQYPPATYDQYPYAASPAT |
| | COL18A1_0 | PGGRVKEGGLKGQKGEPGVPGPPGRAGPPGSPCLP |
| | | GPPGLPCPVSPLGPAGPALQTVPGPQG |
| | COL18A1_1 | CPVSPLGPAGPALQTVPGPQGPPGPPGRDGTPGRDG |
| | | EPGDPGEDGKPGDTGPQGFPGTPGDV |
| | COL18A1_2 | KGEPGDASLGFGMRGMPGPPGPPGPPGPPGTPVYDS |
| | | NVFAESSRPGPPGLPGNQGPPGPKGA |
| | ZMAT4 | DSHYQGKIHAKRLKLLLGEKTPLKTTATPLSPLKPP |
| | | RMDTAPVVASPYQRRDSDRYCGLCAA |
| 1984 | GAB4 | FLGNISSASHGLCSSPAEPSCSHQHLPQEQEPTSEPPV |
| | | SHCVPPTWPIPAPPGCLRSHQHAS |
| 1985 | CT47B1 | LGLIQEAASVQEAASVPEPAVPADLAEMAREPAEEA |
| | | ADEKPPEEAAEEKLTEEATEEPAAEE |
| 1986 | KRTAP16-1_0 | CQDSCGSSSCGPQCRQPSCPVSSCAQPLCCDPVICEP |
| | | SCSVSSGCQPVCCEATTCEPSCSVS |
| 1987 | KRTAP16-1_1 | GSSSCGPQCRQPSCPVSSCAQPLCCDPVICEPSCSVSS |
| | | GCQPVCCEATTCEPSCSVSNCYQP |
| 1988 | KRTAP16-1_2 | QPVCFEATICEPSCSVSNCCQPVCFEATVCEPSCSVS |
| | | SCAQPVCCEPAICEPSCSVSSCCQP |
| 1989 | KRTAP16-1_3 | VSNCCQPVCFEATVCEPSCSVSSCAQPVCCEPAICEP |
| | | SCSVSSCCQPVGSEATSCQPVLCVP |
| 1990 | KRTAP16-1_4 | QPVCFEATVCEPSCSVSSCAQPVCCEPAICEPSCSVS |
| | | SCCQPVGSEATSCQPVLCVPTSCQP |
| 1991 | KRTAP16-1_5 | EATSCQPVLCVPTSCQPVLCKSSCCQPVVCEPSCCS |
| | | AVCTLPSSCQPVVCEPSCCQPVCPTP |
| 1992 | KRTAP16-1_6 | KSSCCQPVVCEPSCCSAVCTLPSSCQPVVCEPSCCQP |
| | | VCPTPTCSVTSSCQAVCCDPSPCEP |
| | KRTAP16-1_7 | EPSCCSAVCTLPSSCQPVVCEPSCCQPVCPTPTCSVT |
| | | SSCQAVCCDPSPCEPSCSESSICQP |
| 1993 | KRTAP16-1_8 | QPVVCEPSCCQPVCPTPTCSVTSSCQAVCCDPSPCEP |
| | | SCSESSICQPATCVALVCEPVCLRP |
| 1994 | KRTAP16-1_9 | QAVCCDPSPCEPSCSESSICQPATCVALVCEPVCLRP |
| | | VCCVQSSCEPPSVPSTCQEPSCCVS |
| 1995 | KRTAP16-1_10 | ESSICQPATCVALVCEPVCLRPVCCVQSSCEPPSVPS |
| | | TCQEPSCCVSSICQPICSEPSPCSP |
| 1996 | KRTAP16-1_11 | VALVCEPVCLRPVCCVQSSCEPPSVPSTCQEPSCCVS |
| | | SICQPICSEPSPCSPAVCVSSPCQP |
| 1997 | KRTAP16-1_12 | VQSSCEPPSVPSTCQEPSCCVSSICQPICSEPSPCSPAV |
| | | CVSSPCQPTCYVVKRCPSVCPEP |
| | KRTAP16-1_13 | EPPSVPSTCQEPSCCVSSICQPICSEPSPCSPAVCVSSP |
| | | CQPTCYVVKRCPSVCPEPVSCPS |
| 1998 | KRTAP16-1_14 | EPSPCSPAVCVSSPCQPTCYVVKRCPSVCPEPVSCPS |
| | | TSCRPLSCSPGSSASAICRPTCPRT |
| | KRTAP16-1_15 | QPTCYVVKRCPSVCPEPVSCPSTSCRPLSCSPGSSAS |
| | | AICRPTCPRTFYIPSSSKRPCSATI |
| | AJM1 | APGPRREDPLGRGRSYENLLGREVREPRGVSPEGRR |
| | | PPVVVNLSTSPRRYAALSLSETSLTE |
| | C11orf91 | GLGPSSERPWPSPWPSGLASIPYEPLRFFYSPPPGPEV |
| | | VASPLVPCPSTPRLASASHPEELC |
| | ADPGK | LLEPELPGSALRSLWSSLCLGPAPAPPGPVSPEGRLA |
| | | AAWDALIVRPVRRWRRVAVGVNACV |
| 1999 | QSOX1 | TNTTPHVPAEGPEASRPPKLHPGLRAAPGQEPPEHM |
| | | AELQRNEQEQPLGQWHLSKRDTGAAL |
| | TGM1 | GDIGGNETVTLRQSFVPVRPGPRQLIASLDSPQLSQV |
| | | HGVIQVDVAPAPGDGGFFSDAGGDS |
| | CACNA1C | LVHHQALAVAGLSPLLQRSHSPASFPRPFATPPATPG |
| | | SRGWPPQPVPTLRLEGVESSEKLNS |
| | F12_0 | AAPPTPVSPRLHVPLMPAQPAPPKPQPTTRTPPQSQT |
| | | PGALPAKREQPPSLTRNGPLSCGQR |
| | F12_1 | VSPRLHVPLMPAQPAPPKPQPTTRTPPQSQTPGALPA |
| | | KREQPPSLTRNGPLSCGQRLRKSLS |
| | DOT1L_0 | KNQTALDALHAQTVSQTAASSPQDAYRSPHSPFYQ |
| | | LPPSVQRHSPNPLLVAPTPPALQKLLE |
| 2000 | DOT1L_1 | IGLAKSADSPLQASSALSQNSLFTFRPALEEPSADAK |
| | | LAAHPRKGFPGSLSGADGLSPGTNP |
| | TCF7L1_0 | FAEVRRPQDSAFFKGPPYPGYPFLMIPDLSSPYLSNG |
| | | PLSPGGARTYLQMKWPLLDVPSSAT |
| 2001 | TCF7L1_1 | HHMHPLTPLITYSNDHFSPGSPPTHLSPEIDPKTGIPR |
| | | PPHPSELSPYYPLSPGAVGQIPHP |
| | TCF7L1_2 | HFSPGSPPTHLSPEIDPKTGIPRPPHPSELSPYYPLSPG |
| | | AVGQIPHPLGWLVPQQGQPMYSL |
| | CBARP_0 | PFLASPPPALGRYFSVDGGARGGPVGPCPPSPPPRRP |
| | | RERSPGPVDTRSPASSGKAPPRGGL |
| | CBARP_1 | GRYFSVDGGARGGPVGPCPPSPPPRRPRERSPGPVD |
| | | TRSPASSGKAPPRGGLTGATSPAWTR |
| 2002 | SHF_0 | GSGGVAKWLREHLGFRGGGGGGGGSKPAPPEPDY |
| | | RPPAPSPAAPPAPPPDILAAYRLQRERD |
| | SHF_1 | FEDPYSGGSSGSAALATPVAPGPTPPPRHGSPPHRLI |
| | | RVETPGPPAPPADERISGPPASSDR |
| | SHF_2 | GSAALATPVAPGPTPPPRHGSPPHRLIRVETPGPPAP |
| | | PADERISGPPASSDRLAILEDYADP |
| 2003 | SHF_3 | SCLSPGREEKGRLPPRLSAGNPKSAKPLSMEPSSPLG |
| | | EWTDPALPLENQVWYHGAISRTDAE |
| | PTOV1 | RSGAGGPLGGRGRPPRPLVVRAVRSRSWPASPRGP |
| | | QPPRIRARSAPPMEGARVFGALGPIGP |
| 2004 | EDIL3 | LADGSFSCECPDGFTDPNCSSVVEVASDEEEPTSAGP |
| | | CTPNPCHNGGTCEISEAYRGDTFIG |
| | HOXB13 | PAVNYAPLDLPGSAEPPKQCHPCPGVPQGTSPAPVP |
| | | YGYFGGGYYSCRVSRSSLKPCAQAAT |
| 2005 | NUDT15 | SFIEKENYHYVTILMKGEVDVTHDSEPKNVEPEKNE |
| | | SWEWVPWEELPPLDQLFWGLRCLKEQ |
| | ELAVL4 | FRLDNLLNMAYGVKRLMSGPVPPSACPPRFSPITIDG |
| | | MTSLVGMNIPGHTGTGWCIFVYNLS |
| 2006 | PDX1 | PPHPFPGALGALEQGSPPDISPYEVPPLADDPAVAHL |
| | | HHHLPAQLALPHPPAGPFPEGAEPG |
| | PNPLA1 | PAQPLASSTPLSLSGMPPVSFPAVHKPPSSTPGSSLPT |
| | | PPPGLSPLSPQQQVQPSGSPARSL |
| | CHRD | VLCACEAPQWGRRTRGPGRVSCKNIKPECPTPACG |
| | | QPRQLPGHCCQTCPQERSSSERQPSGL |
| | ALOX12 | AAPLVMLKMEPNGKLQPMVIQIQPPNPSSPTPTLFLP |
| | | SDPPLAWLLAKSWVRNSDFQLHEIQ |
| 2007 | PM20D1 | GSGTVVTVLQQLANEFPFPVNIILSNPWLFEPLISRF |
| | | MERNPLTNAIIRTTTALTIFKAGVK |
| 2008 | COL8A2_0 | GPPGFSRMGKAGPPGLPGKVGPPGQPGLRGEPGIRG |
| | | DQGLRGPPGPPGLPGPSGITIPGKPG |
| 2009 | COL8A2_1 | MPGLPGPKGDRGPAGVPGLLGDRGEPGEDGEPGEQ |
| | | GPQGLGGPPGLPGSAGLPGRRGPPGPK |
| | COL8A2_2 | GEPGLPGPPGEGRAGEPGTAGPTGPPGVPGSPGITGP |
| | | PGPPGPPGPPGAPGAFDETGIAGLH |
| 2010 | COL17A1 | DRGFPGTPGIPGPLGHPGPQGPKGQKGSVGDPGME |
| | | GPMGQRGREGPMGPRGEAGPPGSGEKG |
| | NLGN4X | HNLNEIFQYVSTTTKVPPPDMTSFPYGTRRSPAKIWP |
| | | TTKRPAITPANNPKHSKDPHKTGPE |
| 2011 | SULF1 | HIGQFGLVKGKSMPYDFDIRVPFFIRGPSVEPGSIVP |
| | | QIVLNIDLAPTILDIAGLDTPPDVD |
| 2012 | ANTXR2 | VRWGDKGSTEEGARLEKAKNAVVKIPEETEEPIRPR |
| | | PPRPKPTHQPPQTKWYTPIKGRLDAL |
| | SMAD1 | RNLGQNEPHMPLNATFPDSFQQPNSHPFPHSPNSSY |
| | | PNSPGSSSSTYPHSPTSSDPGSPFQM |
| | NID2_0 | QGNFLPLQCHGSTGFCWCVDPDGHEVPGTQTPPGS |
| | | TPPHCGPSPEPTQRPPTICERWRENLL |
| | NID2_1 | PLQCHGSTGFCWCVDPDGHEVPGTQTPPGSTPPHCG |
| | | PSPEPTQRPPTICERWRENLLEHYGG |
| 2013 | NDST1_0 | LFIFCLFSVFISAYYLYGWKRGLEPSADAPEPDCGDP |
| | | PPVAPSRLLPLKPVQAATPSRTDPL |
| 2014 | NDST1_1 | LFSVFISAYYLYGWKRGLEPSADAPEPDCGDPPPVA |
| | | PSRLLPLKPVQAATPSRTDPLVLVFV |
| 2015 | RNF38_0 | GRRDRLSRHNSISQDENYHHLPYAQQQAIEEPRAFH |
| | | PPNVSPRLLHPAAHPPQQNAVMVDIH |
| | RNF38_1 | SISQDENYHHLPYAQQQAIEEPRAFHPPNVSPRLLHP |
| | | AAHPPQQNAVMVDIHDQLHQGTVPV |
| 2016 | FAM20C_0 | KHTLRILQDFSSDPSSNLSSHSLEKLPPAAEPAERAL |
| | | RGRDPGALRPHDPAHRPLLRDPGPR |
| 2017 | FAM20C_1 | SSDPSSNLSSHSLEKLPPAAEPAERALRGRDPGALRP |
| | | HDPAHRPLLRDPGPRRSESPPGPGG |
| 2018 | TNFRSF13C | KDAPEPLDKVIILSPGISDATAPAWPPPGEDPGTTPP |
| | | GHSVPVPATELGSTELVTTKTAGPE |
| | NOCT | HSPRRLCSALLQRDAPGLRRLPAPGLRRPLSPPAAVP |
| | | RPASPRLLAAASAASGAARSCSRTV |
| | ZNF746_0 | RPFTCTVCGKSFIRKDHLRKHQRNHAAGAKTPARG |
| | | QPLPTPPAPPDPFKSPASKGPLASTDL |
| 2019 | ZNF746_1 | DHLRKHQRNHAAGAKTPARGQPLPTPPAPPDPFKSP |
| | | ASKGPLASTDLVTDWTCGLSVLGPTD |
| 2020 | SNORC | VPQEPVPTLWNEPAELPSGEGPVESTSPGREPVDTGP |
| | | PAPTVAPGPEDSTAQERLDQGGGSL |
| 2021 | STK19 | SWKRHHLIPETFGVKRRRKRGPVESDPLRGEPGSAR |
| | | AAVSELMQLFPRGLFEDALPPIVLRS |
| | SSH2_0 | KFPDLTVEDLETDALKADMNVHLLPMEELTSPLKD |
| | | PPMSPDPESPSPQPSCQTEISDFSTDR |
| 2022 | SSH2_1 | ETDALKADMNVHLLPMEELTSPLKDPPMSPDPESPS |
| | | PQPSCQTEISDFSTDRIDFFSALEKF |
| | SSH2_2 | KADMNVHLLPMEELTSPLKDPPMSPDPESPSPQPSC |
| | | QTEISDFSTDRIDFFSALEKFVELSQ |
| | ARHGAP39 | TFAPEADGTIFFPERRPSPFLKRAELPGSSSPLLAQPR |
| | | KPSGDSQPSSPRYGYEPPLYEEPP |
| 2023 | NFXL1 | GHLCPAPCHDQALIKQTGRHQPTGPWEQPSEPAFIQ |
| | | TALPCPPCQVPIPMECLGKHEVSPLP |
| | WIPF1_0 | NRMPPPRPDVGSKPDSIPPPVPSTPRPIQSSPHNRGSP |
| | | PVPGGPRQPSPGPTPPPFPGNRGT |
| | WIPF1_1 | PPPVPSTPRPIQSSPHNRGSPPVPGGPRQPSPGPTPPPF |
| | | PGNRGTALGGGSIRQSPLSSSSP |
| | OBSCN_0 | GGSSSSSSSSDNELAPFARAKSLPPSPVTHSPLLHPRG |
| | | FLRPSASLPEEAEASERSTEAPAP |
| | OBSCN_1 | NLSDLYDIKYLPFEFMIFRKVPKSAQPEPPSPMAEEE |
| | | LAEFPEPTWPWPGELGPHAGLEITE |
| 2024 | OBSCN_2 | FEFMIFRKVPKSAQPEPPSPMAEEELAEFPEPTWPWP |
| | | GELGPHAGLEITEESEDVDALLAEA |
| | VWCE_0 | TATFPGEPGASPRLSPGPSTPPGAPTLPLASPGAPQPP |
| | | PVTPERSFSASGAQIVSRWPPLPG |
| | VWCE_1 | GTLLTEASALSMMDPSPSKTPITLLGPRVLSPTTSRL |
| | | STALAATTHPGPQQPPVGASRGEES |
| | PFKFB2_0 | YGCKVETIKLNVEAVNTHRDKPTNNFPKNQTPVRM |
| | | RRNSFTPLSSSNTIRRPRNYSVGSRPL |
| | PFKFB2_1 | NVEAVNTHRDKPTNNFPKNQTPVRMRRNSFTPLSSS |
| | | NTIRRPRNYSVGSRPLKPLSPLRAQD |
| | NCOA6 | MILSRAQLMPQGQMMVNPPSQNLGPSPQRMTPPKQ |
| | | MLSQQGPQMMAPHNQMMGPQGQVLLQQ |
| | CCDC120 | DNEEPHGCFSLAERPSPPKAWDQLRAVSGGSPERRT |
| | | PWKPPPSDLYGDLKSRRNSVASPTSP |
| 2025 | AHDC1 | LLADFLGRTEAACLSAPHLASPPATPKADKEPLEMA |
| | | RPPGPPRGPAAAAAGYGCPLLSDLTL |
| | ATXN7L2 | REVQGRAKDFDVLVAELKANSRKGESPKEKSPGRK |
| | | EQVLERPSQELPSSVQVVAAVAAPSST |
| 2026 | TRIM16 | KSCLTCMVNYCEEHLQPHQVNIKLQSHLLTEPVKD |
| | | HNWRYCPAHHSPLSAFCCPDQQCICQD |
| | STIL | FARPQMNTRFPSSRMVPFHFPPSKCALWNPTPTGDF |
| | | IYLHLSYYRNPKLVVTEKTIRLAYRH |
| 2027 | SCAF4 | TPPFPPMAQPVIPPTPPVQQPFQASFQAQNEPLTQKP |
| | | HQQEMEVEQPCIQEVKRHMSDNRKS |
| | EIF4G3_0 | KQEVLPLTLELEILENPPEEMKLECIPAPITPSTVPSFP |
| | | PTPPTPPASPPHTPVIVPAAATT |
| | EIF4G3_1 | LEILENPPEEMKLECIPAPITPSTVPSFPPTPPTPPASPP |
| | | HTPVIVPAAATTVSSPSAAITV |
| | PRKCQ | RDTEQIFREGPVEIGLPCSIKNEARPPCLPTPGKREPQ |
| | | GISWESPLDEVDKMCHLPEPELNK |
| | SCMH1 | KFPKKRGPKPGSKRKPRTLLNPPPASPTTSTPEPDTS |
| | | TVPQDAATIPSSAMQAPTVCIYLNK |
| | CABIN1 | CLVDEDSHSSAGTLPGPGASLPSSSGPGLTSPPYTAT |
| | | PIDHDYVKCKKPHQQATPDDRSQDS |
| | SMPD4_0 | TSDCAYFILVDRYLSWFLPTEGSVPPPLSSSPGGTSPS |
| | | PPPRTPAIPFASYGLHHTSLLKRH |
| | SMPD4_1 | YFILVDRYLSWFLPTEGSVPPPLSSSPGGTSPSPPPRT |
| | | PAIPFASYGLHHTSLLKRHISHQT |
| 2028 | THAP7_0 | FSDLLGPLGAQADEAGCSAQPSPERQPSPLEPRPVSP |
| | | SAYMLRLPPPAGAYIQNEHSYQVGS |
| | THAP7_1 | GPLGAQADEAGCSAQPSPERQPSPLEPRPVSPSAYM |
| | | LRLPPPAGAYIQNEHSYQVGSALLWK |
| | EIF4G2 | QSFLMNKNQVPKLQPQITMIPPSAQPPRTQTPPLGQT |
| | | PQLGLKTNPPLIQEKPAKTSKKPPP |
| 2029 | AKAP1_0 | RVCQASQLQGQKEESCVPVHQKTVLGPDTAEPATA |
| | | EAAVAPPDAGLPLPGLPAEGSPPPKTY |
| | AKAP1_1 | GPDTAEPATAEAAVAPPDAGLPLPGLPAEGSPPPKT |
| | | YVSCLKSLLSSPTKDSKPNISAHHIS |
| 2030 | TRAF4 | CDTCLQEFLSEGVFKCPEDQLPLDYAKIYPDPELEV |
| | | QVLGLPIRCIHSEEGCRWSGPLRHLQ |
| 2031 | OTUD7B | CVGGLPPYATFPRQCPPGRPYPHQDSIPSLEPGSHSK |
| | | DGLHRGALLPPPYRVADSYSNGYRE |
| 2032 | PRPF8 | FPPFDDEEPPLDYADNILDVEPLEAIQLELDPEEDAP |
| | | VLDWFYDHQPLRDSRKYVNGSTYQR |
| 2033 | CNOT11 | MYRTEPLAANPFAASFAHLLNPAPPARGGQEPDRPP |
| | | LSGFLPPITPPEKFFLSQLMLAPPRE |
| 2034 | MRPL19 | EKRLDDSLLYLRDALPEYSTFDVNMKPVVQEPNQK |
| | | VPVNELKVKMKPKPWSKRWERPNFNIK |
| 2035 | VARS1 | LEKFQQKQKIQQQQPPPGEKKPKPEKREKRDPGVIT |
| | | YDLPTPPGEKKDVSGPMPDSYSPRYV |
| 2036 | FRAS1_0 | LRGISEAGFLDDVVYDSTALGPGYDRPFQFDPSVRE |
| | | PKTIQLYKHLNLKSCVWTFDAYYDMT |
| 2037 | FRAS1_1 | EAGFLDDVVYDSTALGPGYDRPFQFDPSVREPKTIQ |
| | | LYKHLNLKSCVWTFDAYYDMTELIDV |
| | ZNF684_0 | GCPITKTKVILKVEQGQEPWMVEGANPHESSPESDY |
| | | PLVDEPGKHRESKDNFLKSVLLTFNK |
| 2038 | ZNF684_1 | LKVEQGQEPWMVEGANPHESSPESDYPLVDEPGKH |
| | | RESKDNFLKSVLLTFNKILTMERIHHY |
| | RGL2 | PSVSSLDSALESSPSLHSPADPSHLSPPASSPRPSRGH |
| | | RRSASCGSPLSGGAEEASGGTGYG |
| | MAP3K21 | TGATIISATGASALPLCPSPAPHSHLPREVSPKKHSTV |
| | | HIVPQRRPASLRSRSDLPQAYPQT |
| 2039 | PPDPF | RLGSTSSNSSCSSTECPGEAIPHPPGLPKADPGHWW |
| | | ASFFFGKSTLPFMATVLESAEHSEPP |
| 2040 | CRACDL_0 | EEGGVPGEDPSSRPATPELAEPESAPTLRVEPPSPPEG |
| | | PPNPGPDGGKQDGEAPPAGPCAPA |
| 2041 | CRACDL_1 | DTTPPETDPAATSEAPSARDGPERSVPKEAEPTPPVL |
| | | PDEEKGPPGPAPEPEREAETEPERG |
| 2042 | CRACDL_2 | KEAEPTPPVLPDEEKGPPGPAPEPEREAETEPERGAG |
| | | TEPERIGTEPSTAPAPSPPAPKSCL |
| 2043 | CRACDL_3 | SKPPLPRKPLLQSFTLPHQPAPPDAGPGEREPRKEPR |
| | | TAEKRPLRRGAEKSLPPAATGPGAD |
| 2044 | FAM83G | DSRPRPEPCPPPEPSAPQDGVPAENGLPQGDPEPLPP |
| | | VPKPRTVPVADVLARDSSDIGWVLE |
| | MN1_0 | GPQRPGNLPDFHSSGASSHAVPAPCLPLDQSPNRAA |
| | | SFHGLPSSSGSDSHSLEPRRVTNQGA |
| | MN1_1 | RCASWNGSMHNGALDNHLSPSAYPGLPGEFTPPVP |
| | | DSFPSGPPLQHPAPDHQSLQQQQQQQQ |
| 2045 | DSG1 | DISLGKESYPDLDPSWPPQSTEPVCLPQETEPVVSGH |
| | | PPISPHFGTTTVISESTYPSGPGVL |
| | FARP2_0 | PSAQPLGPPALQPGPGLSTKSPQPSPSSRKSPLSLSPA |
| | | FQVPLGPAEQGSSPLLSPVLSDAG |
| | FARP2_1 | LGPPALQPGPGLSTKSPQPSPSSRKSPLSLSPAFQVPL |
| | | GPAEQGSSPLLSPVLSDAGGAGMD |
| | ZNF787 | EDQQMASHENPVDILIMDDDDVPSWPPTKLSPPQSA |
| | | PPAGPPPRPRPPAPYICNECGKSFSH |
| 2046 | PSMF1 | VGGEDLDPFGPRRGGMIVDPLRSGFPRALIDPSSGLP |
| | | NRLPPGAVPPGARFDPFGPIGTSPP |
| | ENKD1 | EPGPASGTESAHFLRAHSRCGPGLPPPHVSSPQPTPP |
| | | GPEAKEPGLGVDFIRHNARAAKRAP |
| | DAXX_0 | TANSIIVLDDDDEDEAAAQPGPSHPLPNAASPGAEA |
| | | PSSSEPHGARGSSSSGGKKCYKLENE |
| 2047 | DAXX_1 | DDEDEAAAQPGPSHPLPNAASPGAEAPSSSEPHGAR |
| | | GSSSSGGKKCYKLENEKLFEEFLELC |
| | HIVEP1_0 | YNIAVTSSVGLTSPSSRSQVTPQNQQMDSASPLSISP |
| | | ANSTQSPPMPIYNSTHVASVVNQSV |
| | HIVEP1_1 | TSSVGLTSPSSRSQVTPQNQQMDSASPLSISPANSTQ |
| | | SPPMPIYNSTHVASVVNQSVEQMCN |
| | HIVEP1_2 | EVSDLRSKSFDCGSITPPQTTPLTELQPPSSPSRVGVT |
| | | GHVPLLERRRGPLVRQISLNIAPD |
| | SETBP1 | RQRGGESDFLPVSSAKPPAAPGCAGEPLLSTPGPGK |
| | | GIPVGGERMEPEEEDELGSGRDVDSN |
| | SRRM2_0 | ATRPSPSPERSSTGPEPPAPTPLLAERHGGSPQPLATT |
| | | PLSQEPVNPPSEASPTRDRSPPKS |
| 2048 | SRRM2_1 | TGPEPPAPTPLLAERHGGSPQPLATTPLSQEPVNPPS |
| | | EASPTRDRSPPKSPEKLPQSSSSES |
| 2049 | PARD3B | LAAFKPIGGEIEVTPSALKLGTPLLVRRSSDPVPGPP |
| | | ADTQPSASHPGGQSLKLVVPDSTQN |
| | MAPK7 | RSLLERWTRMARPAAPALTSVPAPAPAPTPTPTPVQ |
| | | PTSPPPGPVAQPTGPQPQSAGSTSGP |
| 2050 | CPSF6 | PPAPHVNPAFFPPPTNSGMPTSDSRGPPPTDPYGRPP |
| | | PYDRGDYGPPGREMDTARTPLSEAE |
| | ALX3 | LQNSLWASPGSGSPGGPCLVSPEGIPSPCMSPYSHPH |
| | | GSVAGFMGVPAPSAAHPGIYSIHGF |
| | ATXN1L_0 | QLPSTSLQFIGSPYSLPYAVPPNFLPSPLLSPSANLAT |
| | | SHLPHFVPYASLLAEGATPPPQAP |
| | ATXN1L_1 | PSPLLSPSANLATSHLPHFVPYASLLAEGATPPPQAP |
| | | SPAHSFNKAPSATSPSGQLPHHSST |
| | ATXN1L_2 | PYASLLAEGATPPPQAPSPAHSFNKAPSATSPSGQLP |
| | | HHSSTQPLDLAPGRMPIYYQMSRLP |
| | ZZEF1_0 | IRPVDFKQRNKADKGVSLSKDPSCQTQISDSPADAS |
| | | PPTGLPDAEDSEVSSQKPIEEKAVTP |
| | ZZEF1_1 | FKQRNKADKGVSLSKDPSCQTQISDSPADASPPTGL |
| | | PDAEDSEVSSQKPIEEKAVTPSPEQV |
| 2051 | ZNF318_0 | SFDAYRHYMAYAASRWPMYPTSQPSNHPVPEPHRI |
| | | MPITKQATRSRPNLRVIPTVTPDKPKQ |
| | ZNF318_1 | DLKVEELTALGNLGDMPVDFCTTRVSPAHRSPTVL |
| | | CQKVCEENSVSPIGCNSSDPADFEPIP |
| 2052 | KATNB1 | PNLEVLPRPPVVASTPAPKAEPAIIPATRNEPIGLKAS |
| | | DFLPAVKIPQQAELVDEDAMSQIR |
| | PDLIM4 | DPEIQDGSPTTSRRPSGTGTGPEDGRPSLGSPYGQPP |
| | | RFPVPHNGSSEATLPAQMSTLHVSP |
| | CCDC9_0 | VAVTAPRKGRSVEKENVAVESEKNLGPSRRSPGTPR |
| | | PPGASKGGRTPPQQGGRAGMGRASRS |
| | CCDC9_1 | AAPRAYSDHDDRWETKEGAASPAPETPQPTSPETSP |
| | | KETPMQPPEIPAPAHRPPEDEGEENE |
| | CNNM4_0 | VEAGKENMKFETGAFSYYGTMALTSVPSDRSPAHP |
| | | TPLSRSASLSYPDRTDVSTAATLAGSS |
| | CNNM4_1 | ENMKFETGAFSYYGTMALTSVPSDRSPAHPTPLSRS |
| | | ASLSYPDRTDVSTAATLAGSSNQFGS |
| | CSF2RB | YVSSADLVFTPNSGASSVSLVPSLGLPSDQTPSLCPG |
| | | LASGPPGAPGPVKSGFEGYVELPPI |
| 2053 | FHOD1_0 | PETAPAARTPQSPAPCVLLRAQRSLAPEPKEPLIPASP |
| | | KAEPIWELPTRAPRLSIGDLDFSD |
| 2054 | FHOD1_1 | QSPAPCVLLRAQRSLAPEPKEPLIPASPKAEPIWELPT |
| | | RAPRLSIGDLDFSDLGEDEDQDML |
| 2055 | ZNF592_0 | DPHNCGKFDSTFMNGDSARSFPGKLEPPKSEPLPTF |
| | | NQFSPISSPEPEDPIKDNGFGIKPKH |
| 2056 | ZNF592_1 | MAVEVAEPEEGSGEEVPMETRENGLEECAGEPLSA |
| | | DPEARRLLGPAPEDDGGHNDHSQPQAS |
| | SPEG_0 | YMATATNELGQATCAASLTVRPGGSTSPFSSPITSDE |
| | | EYLSPPEEFPEPGETWPRTPTMKPS |
| | SPEG_1 | QATCAASLTVRPGGSTSPFSSPITSDEEYLSPPEEFPE |
| | | PGETWPRTPTMKPSPSQNRRSSDT |
| | SPEG_2 | ARRLQESPSLSALSEAQPSSPARPSAPKPSTPKSAEPS |
| | | ATTPSDAPQPPAPQPAQDKAPEPR |
| 2057 | SPEG_3 | ESPSLSALSEAQPSSPARPSAPKPSTPKSAEPSATTPS |
| | | DAPQPPAPQPAQDKAPEPRPEPVR |
| | SPEG_4 | SALSEAQPSSPARPSAPKPSTPKSAEPSATTPSDAPQP |
| | | PAPQPAQDKAPEPRPEPVRASKPA |
| 2028 | SPEG_5 | STPKSAEPSATTPSDAPQPPAPQPAQDKAPEPRPEPV |
| | | RASKPAPPPQALQTLALPLTPYAQI |
| | SPEG_6 | LSGHAQGPSQGPAAPPSEPKPHAAVFARVASPPPGA |
| | | PEKRVPSAGGPPVLAEKARVPTVPPR |
| | ARHGAP30 | PALQHRPSPASGPGPGPGLGPGPPDEKLEASPASSPL |
| | | ADSGPDDLAPALEDSLSQEVQDSFS |
| 2059 | TNFRSF8 | KFPGTAQKNTVCEPASPGVSPACASPENCKEPSSGTI |
| | | PQAKPTPVSPATSSASTMPVRGGTR |
| 2060 | ETL4 | NRDSVASSSHIAQEASPRPLLVPDEGPTALEPPTSIPS |
| | | ASRKGSSGAPQTSRMPVPMSAKNR |
| | TTBK2 | KIKLGICKAATEEENSHGQANGLLNAPSLGSPIRVRS |
| | | EITQPDRDIPLVRKLRSIHSFELEK |
| | POLR2A_0 | SAASDASGFSPGYSPAWSPTPGSPGSPGPSSPYIPSPG |
| | | GAMSPSYSPTSPAYEPRSPGGYTP |
| | POLR2A_1 | ASGFSPGYSPAWSPTPGSPGSPGPSSPYIPSPGGAMSP |
| | | SYSPTSPAYEPRSPGGYTPQSPSY |
| | POLR2A_2 | AWSPTPGSPGSPGPSSPYIPSPGGAMSPSYSPTSPAYE |
| | | PRSPGGYTPQSPSYSPTSPSYSPT |
| | KLF10 | SAGGVPPMPVICQMVPLPANNPVVTTVVPSTPPSQP |
| | | PAVCPPVVFMGTQVPKGAVMFVVPQP |
| 2061 | LIN37 | PPTPPGPPGDACRSRIPSPLQPEMQGTPDDEPSEPEPS |
| | | PSTLIYRNMQRWKRIRQRWKEASH |
| | ALDOC_0 | VTEKVLAAVYKALSDHHVYLEGTLLKPNMVTPGH |
| | | ACPIKYTPEEIAMATVTALRRTVPPAVP |
| | ALDOC_1 | KALSDHHVYLEGTLLKPNMVTPGHACPIKYTPEEIA |
| | | MATVTALRRTVPPAVPGVTFLSGGQS |
| | NEO1 | VKPPDLWIHHERLELKPIDKSPDPNPIMTDTPIPRNS |
| | | QDITPVDNSMDSNIHQRRNSYRGHE |
| 2062 | DAB2_0 | QSTKPGRGRRTAKSSANDLLASDIFAPPVSEPSGQAS |
| | | PTGQPTALQPNPLDLFKTSAPAPVG |
| | DAB2_1 | PGAMMGGQPSGFSQPVIFGTSPAVSGWNQPSPFAAS |
| | | TPPPVPVVWGPSASVAPNAWSTTSPL |
| | DAB2_2 | SPLGNPFQSNIFPAPAVSTQPPSMHSSLLVTPPQPPPR |
| | | AGPPKDISSDAFTALDPLGDKEIK |
| | GPATCH8_0 | KNSVTAKLLLEKIQSRKVERKPSVSEEVQATPNKAG |
| | | PKLKDPPQGYFGPKLPPSLGNKPVLP |
| 2063 | GPATCH8_1 | EKIQSRKVERKPSVSEEVQATPNKAGPKLKDPPQGY |
| | | FGPKLPPSLGNKPVLPLIGKLPATRK |
| 2064 | GPATCH8_2 | SSSQPGPVESSLLPIAPDLEHFPSYAPPSGDPSIESTDG |
| | | AEDASLAPLESQPITFTPEEMEK |
| | TMEM131_0 | HHAHSPLEQHPQPPLPPPVPQPQEPQPERLSPAPLAH |
| | | PSHPERASSARHSSEDSDITSLIEA |
| | TMEM131_1 | LPFTTPANTLASIGLMGTENSPAPHAPSTSSPADDLG |
| | | QTYNPWRIWSPTIGRRSSDPWSNSH |
| 2065 | DVL2 | NARLPCFNGRVVSWLVSSDNPQPEMAPPVHEPRAE |
| | | LAPPAPPLPPLPPERTSGIGDSRPPSF |
| | DIP2A | NPWSISSCDAFLNVFQSRGLRPEVICPCASSPEALTV |
| | | AIRRPPDLGGPPPRKAVLSMNGLSY |
| | MINK1_0 | ERTRMNKQQNSPLAKSKPGSTGPEPPIPQASPGPPGP |
| | | LSQTPPMQRPVEPQEGPHKSLVAHR |
| | MINK1_1 | SPLAKSKPGSTGPEPPIPQASPGPPGPLSQTPPMQRPV |
| | | EPQEGPHKSLVAHRVPLKPYAAPV |
| 2066 | PPP1R12C | SLQDLSKERRPGGAGGPPIQDEDEGEEGPTEPPPAEP |
| | | RTLNGVSSPPHPSPKSPVQLEEAPF |
| | IGSF9_0 | FSEIVLSAPEGLPTTPAAPGLPPTEIPPPLSPPRGLVAV |
| | | RTPRGVLLHWDPPELVPKRLDGY |
| | IGSF9_1 | GLPTTPAAPGLPPTEIPPPLSPPRGLVAVRTPRGVLLH |
| | | WDPPELVPKRLDGYVLEGRQGSQG |
| | IGSF9_2 | PDSVAKLKLQGSPVPSLRQSLLWGDPAGTPSPHPDP |
| | | PSSRGPLPLEPICRGPDGRFVMGPTV |
| 2067 | IGSF9_3 | SLRQSLLWGDPAGTPSPHPDPPSSRGPLPLEPICRGP |
| | | DGRFVMGPTVAAPQERSGREQAEPR |
| | IGSF9_4 | RTPAQRLARSFDCSSSSPSGAPQPLCIEDISPVAPPPA |
| | | APPSPLPGPGPLLQYLSLPFFREM |
| 2068 | IGSF9_5 | PLPGPGPLLQYLSLPFFREMNVDGDWPPLEEPSPAA |
| | | PPDYMDTRRCPTSSFLRSPETPPVSP |
| | MDC1 | PEAIAQGGQSKTLRSSTVRAMPVPTTPEFQSPVTTD |
| | | QPISPEPITQPSCIKRQRAAGNPGSL |
| 2069 | NCAPH2_0 | LYSRQGEVLASRKDFRMNTCVPHPRGAFMLEPEGM |
| | | SPMEPAGVSPMPGTQKDTGRTEEQPME |
| | NCAPH2_1 | GEVLASRKDFRMNTCVPHPRGAFMLEPEGMSPMEP |
| | | AGVSPMPGTQKDTGRTEEQPMEVSVCR |
| | ANKIB1 | PENCCQRSGVQMPTPPPSGYNAWDTLPSPRTPRTTR |
| | | SSVTSPDEISLSPGDLDTSLCDICMC |
| 2070 | UBN2_0 | AEYPGPEREPEYPREPPRLEPQPYREPARAEPPAPRE |
| | | PAPRSDAQPPSREKPLPQREVSRAE |
| | UBN2_1 | KSNPTPKPTVSPSSSSPNALVAQGSHSSTNSPVHKQP |
| | | SGMNISRQSPTLNLLPSSRTSGLPP |
| | UBN2_2 | SPNALVAQGSHSSTNSPVHKQPSGMNISRQSPTLNL |
| | | LPSSRTSGLPPTKNLQAPSKLTNSSS |
| 2071 | RASAL3_0 | RLSKALWGRHKNPPPEPDPEPEQEAPELEPEPELEPP |
| | | TPQIPEAPTPNVPVWDIGGFTLLDG |
| | RASAL3_1 | EPDPEPEQEAPELEPEPELEPPTPQIPEAPTPNVPVWD |
| | | IGGFTLLDGKLVLLGGEEEGPRRP |
| | TNRC6B_0 | KKKEATQKVTEQKTKVPEVTKPSLSQPTAASPIGSSP |
| | | SPPVNGGNNAKRVAVPNGQPPSAAR |
| | TNRC6B_1 | TQKVTEQKTKVPEVTKPSLSQPTAASPIGSSPSPPVN |
| | | GGNNAKRVAVPNGQPPSAARYMPRE |
| | TNRC6B_2 | GDPNSYNYKNVNLWDKNSQGGPAPREPNLPTPMTS |
| | | KSASVWSKSTPPAPDNGTSAWGEPNES |
| 2072 | MAP3K11 | LDSDDSSPLGSPSTPPALNGNPPRPSLEPEEPKRPVPA |
| | | ERGSSSGTPKLIQRALLRGTALLA |
| 2073 | XAGE2 | WRGRSTYRPRPRRSLQPPELIGAMLEPTDEEPKEEKP |
| | | PTKSRNPTPDQKREDDQGAAEIQVP |
| | CDAN1 | LQEEREMLRKERSKQLQQSPTPTCPTPELGSPLPSRT |
| | | GSLTDEPADPARVSSRQRLELVALV |
| | KLF13_0 | VARILADLNQQAPAPAPAERREGAAARKARTPCRL |
| | | PPPAPEPTSPGAEGAAAAPPSPAWSEP |
| 2074 | KLF13_1 | QAPAPAPAERREGAAARKARTPCRLPPPAPEPTSPG |
| | | AEGAAAAPPSPAWSEPEPEAGLEPER |
| | STK11IP | ELMSSFRERFGRNWLQYRSHLEPSGNPLPATPTTSA |
| | | PSAPPASSQGPDTAPRPSPPQEEARG |
| 2075 | SLC12A7_0 | FTVVPVEAHADGGGDETAERTEAPGTPEGPEPERPS |
| | | PGDGNPRENSPFLNNVEVEQESFFEG |
| | SLC12A7_1 | VEAHADGGGDETAERTEAPGTPEGPEPERPSPGDGN |
| | | PRENSPFLNNVEVEQESFFEGKNMAL |
| | SLC12A7_2 | ETAERTEAPGTPEGPEPERPSPGDGNPRENSPFLNNV |
| | | EVEQESFFEGKNMALFEEEMDSNPM |
| | DENND5A | GSLERILVGELLTSQPEVDERPCRTPPLQQSPSVIRRL |
| | | VTISPNNKPKLNTGQIQESIGEAV |
| | HIP1 | LQYFKRLIQIPQLPENPPNFLRASALSEHISPVVVIPA |
| | | EASSPDSEPVLEKDDLMDMDASQQ |
| | RBM15B_0 | YDRPLKVEPVYLRGGGGSSRRSSSSSAAASTPPPGPP |
| | | APADPLGYLPLHGGYQYKQRSLSPV |
| 2076 | RBM15B_1 | YLRGGGGSSRRSSSSSAAASTPPPGPPAPADPLGYLP |
| | | LHGGYQYKQRSLSPVAAPPLREPRA |
| | DENND4B_0 | LSGRGPKAGGRQDEAGTPRRGLGARLQQLLTPSRH |
| | | SPASRIPPPELPPDLPPPARRSPMDSL |
| | DENND4B_1 | PKAGGRQDEAGTPRRGLGARLQQLLTPSRHSPASRI |
| | | PPPELPPDLPPPARRSPMDSLLHPRE |
| | DENND4B_2 | QQLLTPSRHSPASRIPPPELPPDLPPPARRSPMDSLLH |
| | | PRERPGSTASESSASLGSEWDLSE |
| 2077 | MAP3K10_0 | EEFAEAEDGGSSVPPSPYSTPSYLSVPLPAEPSPGAR |
| | | APWEPTPSAPPARWGHGARRRCDLA |
| | MAP3K10_1 | FAEAEDGGSSVPPSPYSTPSYLSVPLPAEPSPGARAP |
| | | WEPTPSAPPARWGHGARRRCDLALL |
| 2078 | MAP3K10_2 | SSVPPSPYSTPSYLSVPLPAEPSPGARAPWEPTPSAPP |
| | | ARWGHGARRRCDLALLGCATLLGA |
| | MAP3K10_3 | VPPSPYSTPSYLSVPLPAEPSPGARAPWEPTPSAPPA |
| | | RWGHGARRRCDLALLGCATLLGAVG |
| 2079 | MAP3K10_4 | SDGALGQRGPPEPAGHGPGPRDLLDFPRLPDPQALF |
| | | PARRRPPEFPGRPTTLTFAPRPRPAA |
| | PAIP1_0 | AGPAERARHQPPQPKAPGFLQPPPLRQPRTTPPPGA |
| | | QCEVPASPQRPSRPGALPEQTRPLRA |
| | PAIP1_1 | QPKAPGFLQPPPLRQPRTTPPPGAQCEVPASPQRPSR |
| | | PGALPEQTRPLRAPPSSQDKIPQQN |
| 2080 | ASAP3 | SSLSSEAPETPESLGSPASSSSLMSPLEPGDPSQAPPN |
| | | SEEGLREPPGTSRPSLTSGTTPSE |
| 2081 | MINDY4 | LTVERQKTTASSPPHLPSKRLPPWDRARPRDPSEDTP |
| | | AVDGSTDTDRMPLKLYLPGGNSRMT |
| 2082 | RAVER1 | RLPPEPGLSDSYSFDYPSDMGPRRLFSHPREPALGPH |
| | | GPSRHKMSPPPSGFGERSSGGSGGG |
| 2083 | CASKIN2_0 | VSGPSPEPPPLDESPGPKEGATGPRRRTLSEPAGPSEP |
| | | PGPPAPAGPASDTEEEEPGPEGTP |
| | CASKIN2_1 | TESDTVKRRPKCREREPLQTALLAFGVASATPGPAA |
| | | PLPSPTPGESPPASSLPQPEPSSLPA |
| | CASKIN2_2 | EPLQTALLAFGVASATPGPAAPLPSPTPGESPPASSLP |
| | | QPEPSSLPAQGVPTPLAPSPAMQP |
| | CASKIN2_3 | PLPSPTPGESPPASSLPQPEPSSLPAQGVPTPLAPSPA |
| | | MQPPVPPCPGPGLESSAASRWNGE |
| | CASKIN2_4 | TPGESPPASSLPQPEPSSLPAQGVPTPLAPSPAMQPPV |
| | | PPCPGPGLESSAASRWNGETEPPA |
| | TFAP2E | RPDGLGAAAGGARLSSLPQAAYGPAPPLCHTPAAT |
| | | AAAEFQPPYFPPPYPQPPLPYGQAPDA |
| | CD5 | SRNDMCHSLGLTCLEPQKTTPPTTRPPPTTTPEPTAP |
| | | PRLQLVAQSGGQHCAGVVEFYSGSL |
| | DNAJB1 | DGRTIPVVFKDVIRPGMRRKVPGEGLPLPKTPEKRG |
| | | DLIIEFEVIFPERIPQTSRTVLEQVL |
| | PALMD | DEEEEDEGEAEKPSYHPIAPHSQVYQPAKPTPLPRK |
| | | RSEASPHENTNHKSPHKNSISLKEQE |
| | RNF10 | ALGPTSTEGHGALSISPLSRSPGSHADFLLTPLSPTAS |
| | | QGSPSFCVGSLEEDSPFPSFAQML |
| | KMT2C_0 | PIQDSLSQAQTSQPPSPQVFSPGSSNSRPPSPMDPYA |
| | | KMVGTPRPPPVGHSFSRRNSAAPVE |
| 2084 | KMT2C_1 | SYARPLLTPAPLDSGPGPFKTPMQPPPSSQDPYGSVS |
| | | QASRRLSVDPYERPALTPRPIDNFS |
| 2085 | KMT2C_2 | LTPHPAVNESFAHPSRAFSQPGTISRPTSQDPYSQPP |
| | | GTPRPVVDSYSQSSGTARSNTDPYS |
| 2086 | KMT2C_3 | VDSYSQSSGTARSNTDPYSQPPGTPRPTTVDPYSQQ |
| | | PQTPRPSTQTDLFVTPVTNQRHSDPY |
| 2087 | KMT2C_4 | VDPYSQQPQTPRPSTQTDLFVTPVTNQRHSDPYAHP |
| | | PGTPRPGISVPYSQPPATPRPRISEG |
| 2088 | KMT2C_5 | APPGSVVEASSNLRHGNFIPRPDFPGPRHTDPMRRPP |
| | | QGLPNQLPVHPDLEQVPPSQQEQGH |
| | KMT2C_6 | RETPSKAFHQYSNNISTLDVHCLPQLPEKASPPASPPI |
| | | AFPPAFEAAQVEAKPDELKVTVKL |
| | SH2D3A | RTPSFELPDASERPPTYCELVPRVPSVQGTSPSQSCPE |
| | | PEAPWWEAEEDEEEENRCFTRPQA |
| | PRPF6 | HTSVDPRQTQFGGLNTPYPGGLNTPYPGGMTPGLM |
| | | TPGTGELDMRKIGQARNTLMDMRLSQV |
| | CDK13_0 | LQLRPPPEPSTPVSGQDDLIQHQDMRILELTPEPDRP |
| | | RILPPDQRPPEPPEPPPVTEEDLDY |
| 2089 | CDK13_1 | QHQDMRILELTPEPDRPRILPPDQRPPEPPEPPPVTEE |
| | | DLDYRTENQHVPTTSSSLTDPHAG |
| | ARHGAP17 | KPNSQGPPNPMALPSEHGLEQPSHTPPQTPTPPSTPP |
| | | LGKQNPSLPAPQTLAGGNPETAQPH |
| | HIVEP2_0 | SAQLFGSGKLASPSEVVQQVAEKQYPPHRPSPYSCQ |
| | | HSLSFPQHSLPQGVMHSTKPHQSLEG |
| | HIVEP2_1 | SESAELVACTQDKAPSPSETCDSEISEAPVSPEWAPP |
| | | GDGAESGGKPSPSQQVQQQSYHTQP |
| | MAPIS | PTSEAGLSLPLRGPRARRSASPHDVDLCLVSPCEFEH |
| | | RKAVPMAPAPASPGSSNDSSARSQE |
| | ZBTB4_0 | SSSSSSSSSSSSSSSASSSSSSSSSSPPPASPPASSPPRVL |
| | | ELPGVPAAAFSDVLNFIYSAR |
| | ZBTB4_1 | SSSSSSSSSSASSSSSSSSSSPPPASPPASSPPRVLELPG |
| | | VPAAAFSDVLNFIYSARLALPG |
| | ZBTB4_2 | NTLKLYRLLPMRAAKRPYKTYSQGAPEAPLSPTLNT |
| | | PAPVAMPASPPPGPPPAPEPGPPPSV |
| | ZBTB4_3 | YRLLPMRAAKRPYKTYSQGAPEAPLSPTLNTPAPVA |
| | | MPASPPPGPPPAPEPGPPPSVITFAH |
| 2090 | EVX1 | VPPATRERGGGGPEEEPVDGLAGSAAGPGAEPQVA |
| | | GAAMLGPGPPAPSVDSLSGQGQPSSSD |
| | NFATC3_0 | HLPQLQCRDESVSKEQHMIPSPIVHQPFQVTPTPPVG |
| | | SSYQPMQTNVVYNGPTCLPINAASS |
| | NFATC3_1 | PVADQITGQPSSQLQPITYGPSHSGSATTASPAASHP |
| | | LASSPLSGPPSPQLQPMPYQSPSSG |
| | NFATC3_2 | SSQLQPITYGPSHSGSATTASPAASHPLASSPLSGPPS |
| | | PQLQPMPYQSPSSGTASSPSPATR |
| 2091 | RRP1B | RRKKKKKHHLQPENPGPGGAAPSLEQNRGREPEAS |
| | | GLKALKARVAEPGAEATSSTGEESGSE |
| | ZBTB32 | WLRENPGGSEESLRKLPGPLPPAGSLQTSVTPRPSW |
| | | AEAPWLVGGQPALWSILLMPPRYGIP |
| | DPH2 | VVLLSEPACAHALEALATLLRPRYLDLLVSSPAFPQ |
| | | PVGSLSPEPMPLERFGRRFPLAPGRR |
| 2092 | SAPCD2 | GVRAPLAGPSAAARSPEQLCAPAEAAPCPAEPERSQ |
| | | SAALEPSSSADAGAVACRALEADSGD |
| | DMRTC2_0 | KGTTQPQVPSGKENIAPQPQTPHGAVLLAPTPPGKN |
| | | SCGPLLLSHPPEASPLSWTPVPPGPW |
| | DMRTC2_1 | QTPHGAVLLAPTPPGKNSCGPLLLSHPPEASPLSWTP |
| | | VPPGPWVPGHWLPPGFSMPPPVVCR |
| | DMRTC2_2 | AVLLAPTPPGKNSCGPLLLSHPPEASPLSWTPVPPGP |
| | | WVPGHWLPPGFSMPPPVVCRLLYQE |
| | RBM25 | APSVSSASGNATPNTPGDESPCGIIIPHENSPDQQQPE |
| | | EHRPKIGLSLKLGASNSPGQPNSV |
| | AATK_0 | SGGDHPQAEPKLATEAEGTTGPRLPLPSVPSPSQEG |
| | | APLPSEEASAPDAPDALPDSPTPATG |
| 2093 | AATK_1 | PGEVLPPLLQLEGSSPEPSTCPSGLVPEPPEPQGPAKV |
| | | RPGPSPSCSQFFLLTPVPLRSEGN |
| 2094 | WDR6 | GREITCVKRVGTITLGPEYGVPSFMQPDDLEPGSEGP |
| | | DLTDIVITCSEDTTVCVLALPTTTG |
| | GATA5 | QGALLPREQFAAPLGRPVGTSYSATYPAYVSPDVA |
| | | QSWTAGPFDGSVLHGLPGRRPTFVSDF |
| | CC2D1A | ASIRKGNAIDEADIPPPVAIGKGPASTPTYSPAPTQPA |
| | | PRIASAPEPRVTLEGPSATAPASS |
| | NACAD | HGPRSALGGAREVPDAPPAACPEVSQARLLSPAREE |
| | | RGLSGKSTPEPTLPSAVATEASLDSC |
| | CUX2 | VSLNSPSAASSPGLMMSVSPVPSSSAPISPSPPGAPPA |
| | | KVPSASPTADMAGALHPSAKVNPN |
| | BSN_0 | LGASLLTQASTLMSVQPEADTQGQPAPSKGTPKIVF |
| | | NDASKEAGPKPLGSGPGPGPAPGAKT |
| 2095 | BSN_1 | PLPAKASPLSTKASPLPSKASPQAKPLRASEPSKTPSS |
| | | VQEKKTRVPTKAEPMPKPPPETTP |
| 2096 | BSN_2 | SPQAKPLRASEPSKTPSSVQEKKTRVPTKAEPMPKPP |
| | | PETTPTPATPKVKSGVRRAEPATPV |
| | BSN_3 | EPSKTPSSVQEKKTRVPTKAEPMPKPPPETTPTPATP |
| | | KVKSGVRRAEPATPVVKAVPEAPKG |
| | BSN_4 | PSSVQEKKTRVPTKAEPMPKPPPETTPTPATPKVKSG |
| | | VRRAEPATPVVKAVPEAPKGGEAED |
| | BSN_5 | SGGRVIPDVRVTQHFAKETQDPLKLHSSPASPSSASK |
| | | EIGMPFSQGPGTPATTAVAPCPAGL |
| | BSN_6 | GPRATAEFSTQTPSPAPASDMPRSPGAPTPSPMVAQ |
| | | GTQTPHRPSTPRLVWQESSQEAPFMV |
| | BSN_7 | QTRMVHASASTSPLCSPTETQPTTHGYSQTTPPSVSQ |
| | | LPPEPPGPPGFPRVPSAGADGPLAL |
| 2097 | BSN_8 | TAATDPKVEIVRYISAPEKTGRGESLACQTEPDGQA |
| | | QGVAGPQLVGPTAISPYLPGIQIVTP |
| | BSN_9 | GRGESLACQTEPDGQAQGVAGPQLVGPTAISPYLPG |
| | | IQIVTPGPLGRFEKKKPDPLEIGYQA |
| 2098 | CHERP | AIPPTTQPDDSKPPIQMPGSSEYEAPGGVQDPAAAGP |
| | | RGPGPHDQIPPNKPPWFDQPHPVAP |
| | PPRC1_0 | GPLDLYPKLADTIQTNPIPTHLSLVDSAQASPMPVDS |
| | | VEADPTAVGPVLAGPVPVDPGLVDL |
| 2099 | PPRC1_1 | DTIQTNPIPTHLSLVDSAQASPMPVDSVEADPTAVGP |
| | | VLAGPVPVDPGLVDLASTSSELVEP |
| 2100 | PPRC1_2 | DSAQASPMPVDSVEADPTAVGPVLAGPVPVDPGLV |
| | | DLASTSSELVEPLPAEPVLINPVLADS |
| 2101 | PPRC1_3 | DPTAVGPVLAGPVPVDPGLVDLASTSSELVEPLPAE |
| | | PVLINPVLADSAAVDPAVVPISDNLP |
| 2102 | PPRC1_4 | GPVLAGPVPVDPGLVDLASTSSELVEPLPAEPVLINP |
| | | VLADSAAVDPAVVPISDNLPPVDAV |
| 2103 | PPRC1_5 | DLASTSSELVEPLPAEPVLINPVLADSAAVDPAVVPI |
| | | SDNLPPVDAVPSGPAPVDLALVDPV |
| | PPRC1_6 | ISDNLPPVDAVPSGPAPVDLALVDPVPNDLTPVDPV |
| | | LVKSRPTDPRRGAVSSALGGSAPQLL |
| | PPRC1_7 | PSLPETPTGLADIPCLVIPPAPAKKTALQRSPETPLEIC |
| | | LVPVGPSPASPSPEPPVSKPVAS |
| | PPRC1_8 | PETPTGLADIPCLVIPPAPAKKTALQRSPETPLEICLV |
| | | PVGPSPASPSPEPPVSKPVASSPT |
| | PPRCI_9 | LVIPPAPAKKTALQRSPETPLEICLVPVGPSPASPSPE |
| | | PPVSKPVASSPTEQVPSQEMPLLA |
| | PPRC1_10 | PPAPAKKTALQRSPETPLEICLVPVGPSPASPSPEPPV |
| | | SKPVASSPTEQVPSQEMPLLARPS |
| 2104 | PPRC1_11 | AKKTALQRSPETPLEICLVPVGPSPASPSPEPPVSKPV |
| | | ASSPTEQVPSQEMPLLARPSPPVQ |
| | PPRC1_12 | ETPLEICLVPVGPSPASPSPEPPVSKPVASSPTEQVPS |
| | | QEMPLLARPSPPVQSVSPAVPTPP |
| | LMTK2 | DVMLTGDTLSTSLQSSPEVQVPPTSFETEETPRRVPP |
| | | DSLPTQGETQPTCLDVIVPEDCLHQ |
| | ARNT2 | QLNQSQVAWTGSRPPFPGQQIPSQSSKTQSSPFGIGT |
| | | SHTYPADPSSYSPLSSPATSSPSGN |
| | HHEX | YIEDILGRGPAAPTPAPTLPSPNSSFTSLVSPYRTPVY |
| | | EPTPIHPAFSHHSAAALAAAYGPG |
| | TMEM201 | PHPSVGGSPASLFIPSPPSFLPLANQQLFRSPRRTSPSS |
| | | LPGRLSRALSLGTIPSLTRADSG |
| | ALX4_0 | YGAGQQDLATPLESGAGARGSFNKFQPQPSTPQPQP |
| | | PPQPQPQQQQPQPQPPAQPHLYLQRG |
| | ALX4_1 | IQNPSWLGNNGAASPVPACVVPCDPVPACMSPHAH |
| | | PPGSGASSVTDFLSVSGAGSHVGQTHM |
| | MNT_0 | PLAPRQPALVGAPGLSIKEPAPLPSRPQVPTPAPLLP |
| | | DSKATIPPNGSPKPLQPLPTPVLTI |
| | MNT_1 | KEPAPLPSRPQVPTPAPLLPDSKATIPPNGSPKPLQPL |
| | | PTPVLTIAPHPGVQPQLAPQQPPP |
| | MNT_2 | TTHASVIQTVNHVLQGPGGKHIAHIAPSAPSPAVQL |
| | | APATPPIGHITVHPATLNHVAHLGSQ |
| | NFATC4_0 | ASATPFGTDMDFSPPRPPYPSYPHEDPACETPYLSEG |
| | | FGYGMPPLYPQTGPPPSYRPGLRMF |
| | NFATC4_1 | SDPYGGRGSSFSLGLPFSPPAPFRPPPLPASPPLEGPFP |
| | | SQSDVHPLPAEGYNKVGPGYGPG |
| | TRIM33 | DNLLSRYISGSHLPPQPTSTMNPSPGPSALSPGSSGLS |
| | | NSHTPVRPPSTSSTGSRGSCGSSG |
| | RBPMS | PNPSTPLPNTVPQFIAREPYELTVPALYPSSPEVWAP |
| | | YPLYPAELAPALPPPAFTYPASLHA |
| 2105 | FCHSD1_0 | WRGEFGGRVGVFPSLLVEELLGPPGPPELSDPEQML |
| | | PSPSPPSFSPPAPTSVLDGPPAPVLP |
| | FCHSD1_1 | GVFPSLLVEELLGPPGPPELSDPEQMLPSPSPPSFSPP |
| | | APTSVLDGPPAPVLPGDKALDFPG |
| | SKOR1 | SAPSAGGGPDGEQPTGPPSATSSGADGPANSPDGGS |
| | | PRPRRRLGPPPAGRPAFGDLAAEDLV |
| | SMG6 | QYPYTGYNPLQYPVGPTNGVYPGPYYPGYPTPSGQ |
| | | YVCSPLPTSTMSPEEVEQHMRNLQQQE |
| 2106 | HERPUD1_0 | RPRPVQNFPNDGPPPDVVNQDPNNNLQEGTDPETE |
| | | DPNHLPPDRDVLDGEQTSPSFMSTAWL |
| 2017 | HERPUD1_1 | QNFPNDGPPPDVVNQDPNNNLQEGTDPETEDPNHL |
| | | PPDRDVLDGEQTSPSFMSTAWLVFKTF |
| | EHBP1L1_0 | GKEAEGSLTEASLPEAQVASGAGAGAPRASSPEKAE |
| | | EDRRLPGSQAPPALVSSSQSLLEWCQ |
| | EHBP1L1_1 | AAAAEGQAPDPSPAPGPPTAADSQQPPGGSSPSEEPP |
| | | PSPGEEAGLQRFQDTSQYVCAELQA |
| | TAOK2_0 | QPKSLKVRAGQRPPGLPLPIPGALGPPNTGTPIEQQP |
| | | CSPGQEAVLDQRMLGEEEEAVGERR |
| 2108 | TAOK2_1 | ELGWVQGPALTPVPEEEEEEEEGAPIGTPRDPGDGC |
| | | PSPDIPPEPPPTHLRPCPASQLPGLL |
| 2109 | ASPSCR1_0 | KSGQDPQQEQEQERERDPQQEQERERPVDREPVDR |
| | | EPVVCHPDLEERLQAWPAELPDEFFEL |
| 2110 | ASPSCR1_1 | PQQEQEQERERDPQQEQERERPVDREPVDREPVVC |
| | | HPDLEERLQAWPAELPDEFFELTVDDV |
| | ARHGEF5_0 | RKGTVSSQGTEVVFASASVTPPRTPDSAPPSPAEAYP |
| | | ITPASVSARPPVAFPRRETSCAARA |
| | ARHGEF5_1 | GPLPQASDPAVARQHRPLPSTPDSSHHAQATPRWR |
| | | YNKPLPPTPDLPQPHLPPISAPGSSRI |
| | RBM27_0 | LGTPPPLLAARLVPPRNLMGSSIGYHTSVSSPTPLVP |
| | | DTYEPDGYNPEAPSITSSGRSQYRQ |
| 2111 | RBM27_1 | RLVPPRNLMGSSIGYHTSVSSPTPLVPDTYEPDGYNP |
| | | EAPSITSSGRSQYRQFFSRTQTQRP |
| | ANKRD34A_0 | GRGMLSPRAQEEEEKRDVFEFPLPKPPDDPSPSEPLP |
| | | KPPRHPPKPLKRLNSEPWGLVAPPQ |
| | ANKRD34A_1 | PGLLERRGSGTLLLDHISQTRPGFLPPLNVSPHPPIPD |
| | | IRPQPGGRAPSLPAPPYAGAPGSP |
| | ANKHD1 | PHFALLAAQTMQQIRHPRLPMAQFGGTFSPSPNTW |
| | | GPFPVRPVNPGNTNSSPKHNNTSRLPN |
| 2112 | ZNF444 | DSGMIPLAGTAPGAEGPAPGDSQAVRPYKQEPSSPP |
| | | LAPGLPAFLAAPGTTSCPECGKTSLK |
| | EPS8L2 | PVSRQSIRNSQKHSPTSEPTPPGDALPPVSSPHTHRG |
| | | YQPTPAMAKYVKILYDFTARNANEL |
| | HOXD1 | PVALQPAFPLGNGDGAFVSCLPLAAARPSPSPPAAP |
| | | ARPSVPPPAAPQYAQCTLEGAYEPGA |
| 2113 | OGFR_0 | DTEGRTGPKEGTPGSPSETPGPSPAGPAGDEPAESPS |
| | | ETPGPRPAGPAGDEPAESPSETPGP |
| 2114 | OGFR_1 | GPSPAGPAGDEPAESPSETPGPRPAGPAGDEPAESPS |
| | | ETPGPRPAGPAGDEPAESPSETPGP |
| 2115 | OGFR_2 | GPRPAGPAGDEPAESPSETPGPRPAGPAGDEPAESPS |
| | | ETPGPSPAGPTRDEPAESPSETPGP |
| 2116 | OGFR_3 | GPRPAGPAGDEPAESPSETPGPSPAGPTRDEPAESPS |
| | | ETPGPRPAGPAGDEPAESPSETPGP |
| 2117 | OGFR_4 | GPSPAGPTRDEPAESPSETPGPRPAGPAGDEPAESPS |
| | | ETPGPRPAGPAGDEPAESPSETPGP |
| 2118 | OGFR_5 | GPRPAGPAGDEPAESPSETPGPRPAGPAGDEPAESPS |
| | | ETPGPSPAGPTRDEPAKAGEAAELQ |
| | PPARGC1B_0 | QSRSCTELHKHLTSAQCCLQDRGLQPPCLQSPRLPA |
| | | KEDKEPGEDCPSPQPAPASPRDSLAL |
| 2119 | PPARGC1B_1 | HLTSAQCCLQDRGLQPPCLQSPRLPAKEDKEPGEDC |
| | | PSPQPAPASPRDSLALGRADPGAPVS |
| | HUWE1_0 | PAPRGSGTASDDEFENLRIKGPNAVQLVKTTPLKPSP |
| | | LPVIPDTIKEVIYDMLNALAAYHAP |
| | HUWE1_1 | SGTASDDEFENLRIKGPNAVQLVKTTPLKPSPLPVIP |
| | | DTIKEVIYDMLNALAAYHAPEEADK |
| | PTPN3 | VSQNRSPHQESLSENNPAQSYLTQKSSSSVSPSSNAP |
| | | GSCSPDGVDQQLLDDFHRVTKGGST |
| | SLC24A1 | VHHCVVVKPTPAMLTTPSPSLTTALLPEELSPSPSVL |
| | | PPSLPDLHPKGEYPPDLFSVEERRQ |
| | DOCK2 | IISLASMNSDCSTPSKPTSESFDLELASPKTPRVEQEE |
| | | PISPGSTLPEVKLRRSKKRTKRSS |
| | SHARPIN | VRGATVEGQNGSKSNSPPALGPEACPVSLPSPPEAST |
| | | LKGPPPEADLPRSPGNLTEREELAG |
| | KIF13B | TAVPAEEPPGPQQLVSPGRERPDLEAPAPGSPFRVRR |
| | | VRASELRSFSRMLAGDPGCSPGAEG |
| | UNK | GSCPRGPFCAFAHVEQPPLSDDLQPSSAVSSPTQPGP |
| | | VLYMPSAAGDSVPVSPSSPHAPDLS |
| | BRME1 | VETLGVPLQEATELGDPTQADSARPEQSSQSPVQAV |
| | | PGSGDSQPDDPPDRGTGLSASQRASQ |
| | BICRA_0 | NSVFGGAGAASAPTGTPSGQPLAVAPGLGSSPLVPA |
| | | PNVILHRTPTPIQPKPAGVLPPKLYQ |
| | BICRA_1 | TPSGQPLAVAPGLGSSPLVPAPNVILHRTPTPIQPKPA |
| | | GVLPPKLYQLTPKPFAPAGATLTI |
| | BICRA_2 | QPAPQAPPAVSTPLPLGLQQPQAQQPPQAPTPQAAA |
| | | PPQATTPQPSPGLASSPEKIVLGQPP |
| | BICRA_3 | LGLQQPQAQQPPQAPTPQAAAPPQATTPQPSPGLAS |
| | | SPEKIVLGQPPSATPTAILTQDSLQM |
| | BICRA_4 | PAPQIPAAAPLKGPGPSSSPSLPHQAPLGDSPHLPSPH |
| | | PTRPPSRPPSRPQSVSRPPSEPPL |
| | BICRA_5 | PAAAPLKGPGPSSSPSLPHQAPLGDSPHLPSPHPTRPP |
| | | SRPPSRPQSVSRPPSEPPLHPCPP |
| 2120 | GREB1L | KRHRGWYPGSPLPQPGLVVPVPTVRPLSRTEPLLSA |
| | | PVPQTPLTGILQPRPIPAGETVIVPE |
| 2121 | PITPNM1 | SMNNELLSPEFGPVRDPLADGVEGLGRGSPEPSALP |
| | | PQRIPSDMASPEPEGSQNSLQAAPAT |
| 2122 | NCOR1 | PHHRGSTAGEVYRSHLPTHLDPAMPFHRALDPAAA |
| | | AYLFQRQLSPTPGYPSQYQLYAMENTR |
| | MED13_0 | YTPQTHTSFGMPPSSAPPSNSGAGILPSPSTPRFPTPR |
| | | TPRTPRTPRGAGGPASAQGSVKYE |
| | MED13_1 | HTSFGMPPSSAPPSNSGAGILPSPSTPRFPTPRTPRTP |
| | | RTPRGAGGPASAQGSVKYENSDLY |
| | ACACB | ADVNLPAAQLQIAMGVPLHRLKDIRLLYGESPWGV |
| | | TPISFETPSNPPLARGHVIAARITSEN |
| | ERF_0 | AFRGPPLARLPHDPGVFRVYPRPRGGPEPLSPFPVSP |
| | | LAGPGSLLPPQLSPALPMTPTHLAY |
| | ERF_1 | PLARLPHDPGVFRVYPRPRGGPEPLSPFPVSPLAGPG |
| | | SLLPPQLSPALPMTPTHLAYTPSPT |
| | ERF_2 | YPRPRGGPEPLSPFPVSPLAGPGSLLPPQLSPALPMTP |
| | | THLAYTPSPTLSPMYPSGGGGPSG |
| | HIPK1 | QPLQIQSGVLTQGSCTPLMVATLHPQVATITPQYAV |
| | | PFTLSCAAGRPALVEQTAAVLQAWPG |
| 2123 | HIP1R | EGPPNFLRASALAEHIKPVVVIPEEAPEDEEPENLIEI |
| | | STGPPAGEPVVVADLFDQTFGPPN |
| 2124 | PRR12_0 | PMPLQLEAHLRSHGLEPAAPSPRLRPEESLDPPGAM |
| | | QELLGALEPLPPAPGDTGVGPPNSEG |
| | PRR12_1 | GSSAPPPKAPAPPPKPETPEKTTSEKPPEQTPETAMP |
| | | EPPAPEKPSLLRPVEKEKEKEKVTR |
| 2125 | INPP5D_0 | YGSLSSFPKPAPRKDQESPKMPRKEPPPCPEPGILSPS |
| | | IVLTKAQEADRGEGPGKQVPAPRL |
| | INPP5D_1 | SFPKPAPRKDQESPKMPRKEPPPCPEPGILSPSIVLTK |
| | | AQEADRGEGPGKQVPAPRLRSFTC |
| | INPP5D_2 | QGKPKTPVSSQAPVPAKRPIKPSRSEINQQTPPTPTPR |
| | | PPLPVKSPAVLHLQHSKGRDYRDN |
| | INPP5D_3 | TPVSSQAPVPAKRPIKPSRSEINQQTPPTPTPRPPLPV |
| | | KSPAVLHLQHSKGRDYRDNTELPH |
| | SRRT | NFLTDAKRPALPEIKPAQPPGPAQILPPGLTPGLPYP |
| | | HQTPQGLMPYGQPRPPILGYGAGAV |
| | HERC1_0 | TLLGVVKEGSTSAKVQWDEAEITISFPTFWSPSDTPL |
| | | YNLEPCEPLPFDVARFRGLTASVLL |
| 2126 | HERC1_1 | TSAKVQWDEAEITISFPTFWSPSDTPLYNLEPCEPLPF |
| | | DVARFRGLTASVLLDLTYLTGVHE |
| 2127 | ZNF335 | GPEEEDDDDIVDAGAIDDLEEDSDYNPAEDEPRGRQ |
| | | LRLQRPTPSTPRPRRRPGRPRKLPRL |
| | ARAP3_0 | PQAQPPKPVPKPRTVFGGLSGPATTQRPGLSPALGG |
| | | PGVSRSPEPSPRPPPLPTSSSEQSSA |
| | ARAP3_1 | LGAALEMFASENSPEPLSLIQPQDIVCLGVSPPPTDP |
| | | GDRFPFSFELILAGGRIQHFGTDGA |
| 2128 | ARAP3_2 | EMFASENSPEPLSLIQPQDIVCLGVSPPPTDPGDRFPF |
| | | SFELILAGGRIQHFGTDGADSLEA |
| 2129 | RAX | KAPEEGSEPSPPPAPAPAPEYEAPRPYCPKEPGEARP |
| | | SPGLPVGPATGEAKLSEEEQPKKKH |
| 2130 | RHOXF1 | ENGMNRDGGMIPEGGGGNQEPRQQPQPPPEEPAQA |
| | | AMEGPQPENMQPRTRRTKFTLLQVEEL |
| | PERM1_0 | PGPASSGDQMQRLLQGPAPRPPGEPPGSPKSPGHST |
| | | GSQRPPDSPGAPPRSPSRKKRRAVGA |
| 2131 | PERM1_1 | QDPAGVQWPDMCEFFFPDVGAQRSRRRGSPEPLPR |
| | | ADPVPAPIPGDPVPISIPEVYEHFFFG |
| | LNPK | PSAGAAVTARPGQEIRQRTAAQRNLSPTPASPNQGP |
| | | PPQVPVSPGPPKDSSAPGGPPERTVT |
| 2132 | SYDE1_0 | RLRGREKLPRKKSDAKERGHPAQRPEPSPPEPEPQA |
| | | PEGSQAGAEGPSSPEASRSPARGAYL |
| 2133 | SYDE1_1 | LGPGVPGTGEPAGEIWYNPIPEEDPRPPAPEPPGPQP |
| | | GSAESEGLAPQGAAPASPPTKASRT |
| | SYDE1_2 | GPAAGPGGTRSPRAGYLSDGDSPERPAGPPSPTSFRP |
| | | YEVGPAARAPPAALWGRLSLHLYGL |
| 2134 | ZNF462 | RARIIKHQKMYHKNNLKETTAPPPAPAPMPDPVVPP |
| | | VSLQDPCKELPAEVVERSILESMVKP |
| 2135 | CD248_0 | WTEMPGILWMEPTQPPDFALAYRPSFPEDREPQIPY |
| | | PEPTWPPPLSAPRVPYHSSVLSVTRP |
| | CD248_1 | PSQSPTNQTSPISPTHPHSKAPQIPREDGPSPKLALWL |
| | | PSPAPTAAPTALGEAGLAEHSQRD |
| | OFD1 | RSLESEMYLEGLGRSHIASPSPCPDRMPLPSPTESRH |
| | | SLSIPPVSSPPEQKVGLYRRQTELQ |
| 2136 | TPRN | SAPEPRAGPANRLAGSPPGSGQWKPKVESGDPSLHP |
| | | PPSPGTPSATPASPPASATPSQRQCV |
| | CDC27_0 | PLGTGTSILSKQVQNKPKTGRSLLGGPAALSPLTPSF |
| | | GILPLETPSPGDGSYLQNYTNTPPV |
| | CDC27_1 | TKSVFSQSGNSREVTPILAQTQSSGPQTSTTPQVLSP |
| | | TITSPPNALPRRSSRLFTSDSSTTK |
| | CDC27_2 | SQSGNSREVTPILAQTQSSGPQTSTTPQVLSPTITSPP |
| | | NALPRRSSRLFTSDSSTTKENSKK |
| | CDC27_3 | SREVTPILAQTQSSGPQTSTTPQVLSPTITSPPNALPR |
| | | RSSRLFTSDSSTTKENSKKLKMKF |
| | PODXL | STAPSSQETVQPTSPATALRTPTLPETMSSSPTAASTT |
| | | HRYPKTPSPTVAHESNWAKCEDLE |
| | PODXL2_0 | PTADYVFPDLTEKAGSIEDTSQAQELPNLPSPLPKM |
| | | NLVEPPWHMPPREEEEEEEEEEEREK |
| 2137 | PODXL2_1 | AGLSGQHEEVPALPSFPQTTAPSGAEHPDEDPLGSR |
| | | TSASSPLAPGDMELTPSSATLGQEDL |
| | TELO2_0 | RQRMDILDVLTLAAQELSRPGCLGRTPQPGSPSPNT |
| | | PCLPEAAVSQPGSAVASDWRVVVEER |
| | TELO2_1 | ILDVLTLAAQELSRPGCLGRTPQPGSPSPNTPCLPEA |
| | | AVSQPGSAVASDWRVVVEERIRSKT |
| | CNTROB | TKVPLAMASSLFRVPEPPSSHSQGSGPSSGSPERGGD |
| | | GLTFPRQLMEVSQLLRLYQARGWGA |
| | CIZ1_0 | QFAMPPATYDTAGLTMPTATLGNLRGYGMASPGL |
| | | AAPSLTPPQLATPNLQQFFPQATRQSLL |
| | CIZ1_1 | MPTATLGNLRGYGMASPGLAAPSLTPPQLATPNLQ |
| | | QFFPQATRQSLLGPPPVGVPMNPSQFN |
| 2138 | CIZ1_2 | DIAKEKRTPAPEPEPCEASELPAKRLRSSEEPTEKEPP |
| | | GQLQVKAQPQARMTVPKQTQTPDL |
| 2139 | CIZ1_3 | KRTPAPEPEPCEASELPAKRLRSSEEPTEKEPPGQLQ |
| | | VKAQPQARMTVPKQTQTPDLLPEAL |
| | NUP98 | GSHELENHQIADSMEFGFLPNPVAVKPLTESPFKVH |
| | | LEKLSLRQRKPDEDMKLYQTPLELKL |
| 2140 | PPP1R35_0 | ELKSADGEEAAAVPGPPPEPQVPQLRAPVPEPGLDL |
| | | SLSPRPDSPQPRHGSPGRRKGRAERR |
| 2141 | PPP1R35_1 | LQVPEEQVLNAALREKLALLPPQARAPHPKEPPGPG |
| | | PDMTILCDPETLFYESPHLTLDGLPP |
| | MEF2D | NQSSLQFSNPSGSLVTPSLVTSSLTDPRLLSPQQPAL |
| | | QRNSVSPGLPQRPASAGAMLGGDLN |
| | HMX3 | FALSQVGDLAFPRFEIPAQRFALPAHYLERSPAWWY |
| | | PYTLTPAGGHLPRPEASEKALLRDSS |
| | FOXB1 | GDYSAYGVPLKPLCHAAGQTLPAIPVPIKPTPAAVP |
| | | ALPALPAPIPTLLSNSPPSLSPTSSQ |
| | USP43 | SPPRPQPGHCDGDGEGGFACAPGPVPAAPGSPGEER |
| | | PPGPQPQLQLPAGDGARPPGAQGLKN |
| | MLXIPL_0 | PMAPPTALLQEEPLFSPRFPFPTVPPAPGVSPLPAPAA |
| | | FPPTPQSVPSPAPTPFPIELLPLG |
| | MLXIPL_1 | VSSTLLRSPGSPQETVPEFPCTFLPPTPAPTPPRPPPGP |
| | | ATLAPSRPLLVPKAERLSPPAPS |
| | SLX4 | PGAHRPKGPAKTKGPRHQRKHHESITPPSRSPTKEA |
| | | PPGLNDDAQIPASQESVATSVDGSDS |
| 2142 | SCAP_0 | AAQVTEQSPLGEGALAPMPVPSGMLPPSHPDPAFSIF |
| | | PPDAPKLPENQTSPGESPERGGPAE |
| | SCAP_1 | MLPPSHPDPAFSIFPPDAPKLPENQTSPGESPERGGPA |
| | | EVVHDSPVPEVTWGPEDEELWRKL |
| | RPAP1_0 | LQDHRDVVMLDNLPDLPPALVPSPPKRARPSPGHCL |
| | | PEDEDPEERLRRHDQHITAVLTKIIE |
| 2143 | RPAP1_1 | SPARASLLASQALHRGELQRVPTLLLPMPTEPLLPTD |
| | | WPFLPLIRLYHRASDTPSGLSPTDT |
| | IQSEC2_0 | SYSHPHHPQSPLSPHSPIPPHPSYPPLPPPSPHTPHSPL |
| | | PPTSPHGPLHASGPPGTANPPSA |
| | IQSEC2_1 | HPHHPQSPLSPHSPIPPHPSYPPLPPPSPHTPHSPLPPT |
| | | SPHGPLHASGPPGTANPPSANPK |
| | IQSEC2_2 | SPHSPIPPHPSYPPLPPPSPHTPHSPLPPTSPHGPLHAS |
| | | GPPGTANPPSANPKAKPSRISTV |
| 2144 | MTF1 | GDAESVSDVPPSTGNSASLSLPLVLQPGLSEPPQPLL |
| | | PASAPSAPPPAPSLGPGSQQAAFGN |
| 2145 | MLXIP | PSLAHMDEQGCEHTSRTEDPFIQPTDFGPSEPPLSVP |
| | | QPFLPVFTMPLLSPSPAPPPISPVL |
| 2146 | SPINDOC | NKKPRGQRWKEPPGEEPVRKKRGRPMTKNLDPDPE |
| | | PPSPDSPTETFAAPAEVRHFTDGSFPA |
| | PDLIM7_0 | GTEFMQDPDEEHLKKSSQVPRTEAPAPASSTPQEPW |
| | | PGPTAPSPTSRPPWAVDPAFAERYAP |
| | PDLIM7_1 | LKKSSQVPRTEAPAPASSTPQEPWPGPTAPSPTSRPP |
| | | WAVDPAFAERYAPDKTSTVLTRHSQ |
| 2147 | PDLIM7_2 | EAPAPASSTPQEPWPGPTAPSPTSRPPWAVDPAFAE |
| | | RYAPDKTSTVLTRHSQPATPTPLQSR |
| 2148 | TCFL5 | AKPAVRVRLEDRFNSIPAEPPPAPRGPEPPEPGGALN |
| | | NLVTLIRHPSELMNVPLQQQNKCTA |
| | ZC3H12D | AALRGSFSRLAFSDDLGPLGPPLPVPACSLTPRLGGP |
| | | DWVSAGGRVPGPLSLPSPESQFSPG |
| | IRX5 | TAPSPGYNSHLQYGADPAAAAAAAFSSYVGSPYDH |
| | | TPGMAGSLGYHPYAAPLGSYPYGDPAY |
| | TACC2_0 | HRDASSIGSVGLGGFCTASESSASLDPCLVSPEVTEP |
| | | RKDPQGARGPEGSLLPSPPPSQERE |
| 2149 | TACC2_1 | SIGSVGLGGFCTASESSASLDPCLVSPEVTEPRKDPQ |
| | | GARGPEGSLLPSPPPSQEREHPSSS |
| 2150 | TACC2_2 | PQTGMRGTKPNQVVCVAAGGQPEGGLPVSPEPSLL |
| | | TPTEEAHPASSLASFPAAQIPIAVEEP |
| | TACC2_3 | RGTKPNQVVCVAAGGQPEGGLPVSPEPSLLTPTEEA |
| | | HPASSLASFPAAQIPIAVEEPGSSSR |
| | TACC2_4 | DNQQENPPPTKKIGKKPVAKMPLRRPKMKKTPEKL |
| | | DNTPASPPRSPAEPNDIPIAKGTYTFD |
| 2151 | PRDM10 | KRKAHILKNHPGAELPPSIRKLRPAGPGEPDPMLSTH |
| | | TQLTGTIATPPVCCPHCSKQYSSKT |
| 2152 | CACTIN | LYKLKQEQGVESEPLFPILKQEPQSPSRSLEPEDAAP |
| | | TPPGPSSEGGPAEAEVDGATPTEGD |
| | ANKLE2 | SPSDRQSWPSPAVKGRFKSQLPDLSGPHSYSPGRNS |
| | | VAGSNPAKPGLGSPGRYSPVHGSQLR |
| | RAPIGAP2 | AGEGEAMEEGDSGGSQPSTTSPFKQEVFVYSPSPSSE |
| | | SPSLGAAATPIIMSRSPTDAKSRNS |
| | SLC26A9_0 | ENAPPTDPNNNQTPANGTSVSYITFSPDSSSPAQSEP |
| | | PASAEAPGEPSDMLASVPPFVTFHT |
| 2153 | SLC26A9_1 | TDPNNNQTPANGTSVSYITFSPDSSSPAQSEPPASAE |
| | | APGEPSDMLASVPPFVTFHTLILDM |
| 2154 | GSE1 | MGRPPVPAEAEHRPESTTRPGPNRHEPGGRDPPQHF |
| | | GGPPPLISPKPQLHAAPTALWNPVSL |
| | MAP1A_0 | SQYGTPVFSAPGHALHPGEPALGEAEERCLSPDDST |
| | | VKMASPPPSGPPSATHTPFHQSPVEE |
| | MAP1A_1 | SSPQKGLEVERWLAESPVGLPPEEEDKLTRSPFEIISP |
| | | PASPPEMVGQRVPSAPGQESPIPD |
| | MAP1A_2 | HMKNEPTTPSWLADIPPWVPKDRPLPPAPLSPAPGP |
| | | PTPAPESHTPAPFSWGTAEYDSVVAA |
| | MAP1A_3 | TPSWLADIPPWVPKDRPLPPAPLSPAPGPPTPAPESH |
| | | TPAPFSWGTAEYDSVVAAVQEGAAE |
| 2155 | MAP1A_4 | SSPISPKSLQSDTPTFSYAALAGPTVPPRPEPGPSMEP |
| | | SLTPPAVPPRAPILSKGPSPPLNG |
| | MAP1A_5 | SDTPTFSYAALAGPTVPPRPEPGPSMEPSLTPPAVPP |
| | | RAPILSKGPSPPLNGNILSCSPDRR |
| | MAP1A_6 | RFSPSLEAAEQESGELDPGMEPAAHSLWDLTPLSPA |
| | | PPASLDLALAPAPSLPGDMGDGILPC |
| 2156 | DOCK4_0 | LLSDKHKHSRENSCLSPRERPCSAIYPTPVEPSQRML |
| | | FNHIGDGALPRSDPNLSAPEKAVNP |
| | DOCK4_1 | TQTASPARHTTSVSPSPAGRSPLKGSVQSFTPSPVEY |
| | | HSPGLISNSPVLSGSYSSGISSLSR |
| 2157 | DOCK4_2 | SKTPPPYSVYERTLRRPVPLPHSLSIPVTSEPPALPPK |
| | | PLAARSSHLENGARRTDPGPRPRP |
| | CEP350 | LDSTAHTAKQDTVELQNQKSSAPVHAPRSHSPVKR |
| | | KPDKITANEDPPVISKRRHYDTDEVRQ |
| | MAML2 | PFNIDLGQQSQRSTPRPSLPMEKIVIKSEYSPGLTQGP |
| | | SGSPQLRPPSAGPAFSMANSALST |
| | ATAD5 | FFNSYYIGKSPKKISSPKKVVTSPRKVPPPSPKSSGPK |
| | | RALPPKTLANYFKVSPKPKNNEEI |
| | SMAP2 | PVPEKKLEPVVFEKVKMPQKKEDPQLPRKSSPKSTA |
| | | PVMDLLGLDAPVACSIANSKTSNTLE |
| | PTPN23_0 | GPTQLIQPRAPGPHAMPVAPGPALYPAPAYTPELGL |
| | | VPRSSPQHGVVSSPYVGVGPAPPVAG |
| | PTPN23_1 | GPQAAPLTIRGPSSAGQSTPSPHLVPSPAPSPGPGPVP |
| | | PRPPAAEPPPCLRRGAAAADLLSS |
| | PTPN23_2 | QDLVLGGDVPISSIQATIAKLSIRPPGGLESPVASLPG |
| | | PAEPPGLPPASLPESTPIPSSSPP |
| 2158 | PTPN23_3 | ISSIQATIAKLSIRPPGGLESPVASLPGPAEPPGLPPAS |
| | | LPESTPIPSSSPPPLSSPLPEAP |
| | PTPN23_4 | LPGPAEPPGLPPASLPESTPIPSSSPPPLSSPLPEAPQPK |
| | | EEPPVPEAPSSGPPSSSLELLA |
| | CASC3_0 | HGDSPAPLPPQGMLVQPGMNLPHPGLHPHQTPAPLP |
| | | NPGLYPPPVSMSPGQPPPQQLLAPTY |
| | CASC3_1 | GMNLPHPGLHPHQTPAPLPNPGLYPPPVSMSPGQPP |
| | | PQQLLAPTYFSAPGVMNFGNPSYPYA |
| | GOLGA3 | KVQCAEVNRASTEGESPDGPGQGGLCQNGPTPPFPD |
| | | PPSSLDPTTSPVGPDASPGVAGFHDN |
| 2159 | INF2 | KEGAQRKWAALKEKLGPQDSDPTEANLESADPELC |
| | | IRLLQMPSVVNYSGLRKRLEGSDGGWM |
| | MISP_0 | RRLCDLERERWAVIQGQAVRKSSTVATLQGTPDHG |
| | | DPRTPGPPRSTPLEENVVDREQIDFLA |
| 2160 | MISP_1 | LERERWAVIQGQAVRKSSTVATLQGTPDHGDPRTP |
| | | GPPRSTPLEENVVDREQIDFLAARQQF |
| | MISP_2 | GQAVRKSSTVATLQGTPDHGDPRTPGPPRSTPLEEN |
| | | VVDREQIDFLAARQQFLSLEQANKGA |
| | PROSER2_0 | PPDPPAPETLLAPPPLPSTPDPPRRELRAPSPPVEHPR |
| | | LLRSVPTPLVMAQKISERMAGNEA |
| | PROSER2_1 | MAQKISERMAGNEALSPTSPFREGRPGEWRTPAAR |
| | | GPRSGDPGPGPSHPAQPKAPRFPSNII |
| 2161 | PROSER2_2 | GNEALSPTSPFREGRPGEWRTPAARGPRSGDPGPGP |
| | | SHPAQPKAPRFPSNIIVINGAAREPR |
| | DTL | EDLSKDSLGPTKSSKIEGAGTSISEPPSPISPYASESCG |
| | | TLPLPLRPCGEGSEMVGKENSSP |
| | TOX4_0 | YLKALAAYKDNQECQATVETVELDPAPPSQTPSPPP |
| | | MATVDPASPAPASIEPPALSPSIVVN |
| 2162 | TOX4_1 | NQECQATVETVELDPAPPSQTPSPPPMATVDPASPA |
| | | PASIEPPALSPSIVVNSTLSSYVANQ |
| 2163 | TOX4_2 | VELDPAPPSQTPSPPPMATVDPASPAPASIEPPALSPS |
| | | IVVNSTLSSYVANQASSGAGGQPN |
| | TOX4_3 | APPSQTPSPPPMATVDPASPAPASIEPPALSPSIVVNS |
| | | TLSSYVANQASSGAGGQPNITKLI |
| | TOX4_4 | IKSVPLPTLKMQTTLVPPTVESSPERPMNNSPEAHTV |
| | | EAPSPETICEMITDVVPEVESPSQM |
| | CASKIN1_0 | GPAPATAKVKPTPQLLPPTERPMSPRSLPQSPTHRGF |
| | | AYVLPQPVEGEVGPAAPGPAPPPVP |
| | CASKIN1_1 | PPPEGEARKPAKPPVSPKPVLTQPVPKLQGSPTPTSK |
| | | KVPLPGPGSPEVKRAHGTPPPVSPK |
| | CASKIN1_2 | PEGEARKPAKPPVSPKPVLTQPVPKLQGSPTPTSKK |
| | | VPLPGPGSPEVKRAHGTPPPVSPKPP |
| | CASKIN1_3 | VAGLPSGSAGPSPAPSPARQPPAALAKPPGTPPSLGA |
| | | SPAKPPSPGAPALHVPAKPPRAAAA |
| | SRGAP3 | RLRSDGAAIPRRRSGGDTHSPPRGLGPSIDTPPRAAA |
| | | CPSSPHKIPLTRGRIESPEKRRMAT |
| | CSTF2T | PPLMQTPIQGGIPAPGPIPAAVPGAGPGSLTPGGAMQ |
| | | PQLGMPGVGPVPLERGQVQMSDPRA |
| 2164 | EGR3 | NLFPMIPDYNLYHHPNDMGSIPEHKPFQGMDPIRVN |
| | | PPPITPLETIKAFKDKQIHPGFGSLP |
| | ADNP2 | TQPVGPINRPVGPGVLPVSPSVTPGVLQAVSPGVLS |
| | | VSRAVPSGVLPAGQMTPAGQMTPAGV |
| 2165 | ARHGAP23_0 | HALSFRDSPFGGLPTFNLAQSPASFPPEASEPPRVVR |
| | | PEPSTRALEPPAEDRGDEVVLRQKP |
| 2166 | ARHGAP23_1 | LMPCDTLARRRLARGRPDGEGAGRGGPRAPEPPGS |
| | | ASSSSQESLRPPAAALASRPSRMEALR |
| | PRR36_0 | PKPKGLQALRPPQVTPPRKDAAPALGPLSSSPLATPS |
| | | PSGTKARPVPPPDNAATPLPATLPP |
| | PRR36_1 | HSSSLTCQLATPLPLAPPSPSAPPSLQTLPSPPATPPSQ |
| | | VPPTQLIMSFPEAGVSSLATAAF |
| | PRR36_2 | ASVSPSVSSPLQSMPPTQANPALPSLPTLLSPLATPPL |
| | | SAMSPLQGPVSPATSLGNSAFPLA |
| | PRR36_3 | LQGPVSPATSLGNSAFPLAALPQPGLSALTTPPPQAS |
| | | PSPSPPSLQATPHTLATLPLQDSPL |
| | PRR36_4 | ETPPCPAPCPLQAPPSPLTTPPPETPSSIATPPPQAPPA |
| | | LASPPLQGLPSPPLSPLATPPPQ |
| | PRR36_5 | ETPSSIATPPPQAPPALASPPLQGLPSPPLSPLATPPPQ |
| | | APPALALPPLQAPPSPPASPPLS |
| | PRR36_6 | SIATPPPQAPPALASPPLQGLPSPPLSPLATPPPQAPPA |
| | | LALPPLQAPPSPPASPPLSPLAT |
| | PRR36_7 | PSPQAPNALAVHLLQAPFSPPPSPPVQAPFSPPASPPV |
| | | SPSATPPSQAPPSLAAPPLQVPPS |
| | PRR36_8 | LAVHLLQAPFSPPPSPPVQAPFSPPASPPVSPSATPPS |
| | | QAPPSLAAPPLQVPPSPPASPPMS |
| | PRR36_9 | PSATPPSQAPPSLAAPPLQVPPSPPASPPMSPSATPPP |
| | | QAPPPLAAPPLQVPPSPPASPPMS |
| | PRR36_10 | PSATPPPQAPPPLAAPPLQVPPSPPASPPMSPSATPPP |
| | | RVPPLLAAPPLQVPPSPPASLPMS |
| | PRR36_11 | PSATPPPRVPPLLAAPPLQVPPSPPASLPMSPLAKPPP |
| | | QAPPALATPPLQALPSPPASFPGQ |
| | PRR36_12 | PPLQVPPSPPASLPMSPLAKPPPQAPPALATPPLQALP |
| | | SPPASFPGQAPFSPSASLPMSPLA |
| | PRR36_13 | LATPPLQALPSPPASFPGQAPFSPSASLPMSPLATPPP |
| | | QAPPVLAAPLLQVPPSPPASPTLQ |
| | SOX18_0 | APGHGAAADTRGLAAGPAALAAPAAPASPPSPQRS |
| | | PPRSPEPGRYGLSPAGRGERQAADESR |
| | SOX18_1 | GGCYGAPLAEALRTAPPAAPLAGLYYGTLGTPGPY |
| | | PGPLSPPPEAPPLESAEPLGPAADLWA |
| | SOX18_2 | EALRTAPPAAPLAGLYYGTLGTPGPYPGPLSPPPEAP |
| | | PLESAEPLGPAADLWADVDLTEFDQ |
| | DDI2 | QKENADPRPPVQFPNLPRIDFSSIAVPGTSSPRQRQPP |
| | | GTQQSHSSPGEITSSPQGLDNPAL |
| 2167 | EEFSEC_0 | TLDLGFSCFSVPLPARLRSSLPEFQAAPEAEPEPGEPL |
| | | LQVTLVDCPGHASLIRTIIGGAQI |
| 2168 | EEFSEC_1 | FSCFSVPLPARLRSSLPEFQAAPEAEPEPGEPLLQVTL |
| | | VDCPGHASLIRTIIGGAQIIDLMM |
| 2169 | TRIM47_0 | RKNHTLSELLQLRQGSGPGSGPGPAPALAPEPSAPS |
| | | ALPSVPEPSAPCAPEPWPAGEEPVRC |
| 2170 | TRIM47_1 | RQGSGPGSGPGPAPALAPEPSAPSALPSVPEPSAPCA |
| | | PEPWPAGEEPVRCDACPEGAALPAA |
| | TRIM47_2 | CPEGAALPAALSCLSCLASFCPAHLGPHERSPALRG |
| | | HRLVPPLRRLEESLCPRHLRPLERYC |
| | SF3B2 | AHKVPPPWLIAMQRYGPPPSYPNLKIPGLNSPIPESC |
| | | SFGYHAGGWGKPPVDETGKPLYGDV |
| | TBC1D25 | LLSDWDLSTAFATASKPYLQLRVDIRPSEDSPLLED |
| | | WDIISPKDVIGSDVLLAEKRSSLTTA |
| 2171 | SMPD1 | APGAPVSRILFLTDLHWDHDYLEGTDPDCADPLCCR |
| | | RGSGLPPASRPGAGYWGEYSKCDLPL |
| | HCFC1_0 | SADGKPTTIITTTQASGAGTKPTILGISSVSPSTTKPG |
| | | TTTIIKTIPMSAIITQAGATGVTS |
| 2172 | HCFC1_1 | QTSATSTTMTVMATGAPCSAGPLLGPSMAREPGGR |
| | | SPAFVQLAPLSSKVRLSSPSIKDLPAG |
| | NEUROD6_0 | TPPGHGTLDNSKSMKPYNYCSAYESFYESTSPECAS |
| | | PQFEGPLSPPPINYNGIFSLKQEETL |
| | NEUROD6_1 | GTLDNSKSMKPYNYCSAYESFYESTSPECASPQFEG |
| | | PLSPPPINYNGIFSLKQEETLDYGKN |
| | PPP1R3D_0 | SRKLGPRSLSCLSDLDGGVALEPRACRPPGSPGRAPP |
| | | PTPAPSGCDPRLRPIILRRARSLPS |
| 2173 | PPP1R3D_1 | DGGVALEPRACRPPGSPGRAPPPTPAPSGCDPRLRPII |
| | | LRRARSLPSSPERRQKAAGAPGAA |
| 2174 | NAPRT_0 | GSPLMDMLQLAEEPVPQAGQELRVWPPGAQEPCTV |
| | | RPAQVEPLLRLCLQQGQLCEPLPSLAE |
| 2175 | NAPRT_1 | AEEPVPQAGQELRVWPPGAQEPCTVRPAQVEPLLR |
| | | LCLQQGQLCEPLPSLAESRALAQLSLS |
| 2176 | PPIL4 | DIIKKINETFVDKDFVPYQDIRINHTVILDDPFDDPPD |
| | | LLIPDRSPEPTREQLDSGRIGADE |
| 2177 | CACNA1I_0 | SSAAAPAAEPGVTTEQPGPRSPPSSPPGLEEPLDGAD |
| | | PHVPHPDLAPIAFFCLRQTTSPRNW |
| 2178 | CACNA1I_1 | ALGLYQALQSRRQALGPEAPAPAKPGPHAKEPRHY |
| | | HGKTKGQGDEGRHLGSRHCQTLHGPAS |
| | CACNA1I_2 | NFLCEMEEIPFNPVRSWLKHDSSQAPPSPFSPDASSP |
| | | LLPMPAEFFHPAVSASQKGPEKGTG |
| | CACNA1I_3 | MEEIPFNPVRSWLKHDSSQAPPSPFSPDASSPLLPMP |
| | | AEFFHPAVSASQKGPEKGTGTGTLP |
| 2179 | CACNA1I_4 | SSLAAPGRPHAAALAHGLARSPSWAADRSKDPPGR |
| | | APLPMGLGPLAPPPQPLPGELEPGDAA |
| | ZFPM1 | LLLGAPLAGPGVEARTPADRGPSPAPAPAASPQPGS |
| | | RGPRDGLGPEPQEPPPGPPPSPAAAP |
| | SETDIA_0 | PVPERVAGSPVTPLPEQEASPARPAGPTEESPPSAPL |
| | | RPPEPPAGPPAPAPRPDERPSSPIP |
| 2180 | SETD1A_1 | VTPLPEQEASPARPAGPTEESPPSAPLRPPEPPAGPPA |
| | | PAPRPDERPSSPIPLLPPPKKRRK |
| | KEL | SLNFNRTLRLLMSQYGHFPFFRAYLGPHPASPHTPVI |
| | | QIDQPEFDVPLKQDQEQKIYAQIFR |
| | CCDC102A_0 | ESPQLSKGSLLTILGSPSPERMGPADSLPPTPPSGTPS |
| | | PGPPPALPLPPAPALLADGDWESR |
| | CCDC102A_1 | GSLLTILGSPSPERMGPADSLPPTPPSGTPSPGPPPAL |
| | | PLPPAPALLADGDWESREELRLRE |
| | NIBAN2 | TEIRGLLAQGLRPESPPPAGPLLNGAPAGESPQPKAA |
| | | PEASSPPASPLQHLLPGKAVDLGPP |
| 2181 | FAM89B | CQDLSFCQDLSSSLHSDSSYPPDAGLSDDEEPPDASL |
| | | PPDPPPLTVPQTHNARDQWLQDAFH |
| 2182 | SETX | RMGIEVKGGIFLWDPQPSSPQHPGATPPTGEPGFPV |
| | | VHQDLSHIQQPAAVVAALSSHKPPVR |
| | TANC2_0 | EEEYLEQDVENVSIGLQTEARPSQGLPVIQSPPSSPPH |
| | | RDSAYISSSPLGSHQVFDFRSSSS |
| | TANC2_1 | SSSQLGSPDVSHLIRRPISVNPNEIKPHPPTPRPLLHSQ |
| | | SVGLRFSPSSNSISSTSNLTPTF |
| | EPOP_0 | ASAPPRPAPGLEPQRGPAASPPQEPSSRPPSPPAGLST |
| | | EPAGPGTAPRPFLPGQPAEVDGNP |
| 2183 | EPOP_1 | PGLEPQRGPAASPPQEPSSRPPSPPAGLSTEPAGPGT |
| | | APRPFLPGQPAEVDGNPPPAAPEAP |
| | EPOP_2 | PGTAPRPFLPGQPAEVDGNPPPAAPEAPAASPSTASP |
| | | APAAPGDLRQEHFDRLIRRSKLWCY |
| | EPOP_3 | RPFLPGQPAEVDGNPPPAAPEAPAASPSTASPAPAAP |
| | | GDLRQEHFDRLIRRSKLWCYAKGFA |
| | ICE1_0 | GSTEFVDHDHFFDEDLQAAIDFFKLPPPLLSPVPSPPP |
| | | MSSPHPGSLPSSFAPETYFGEYTD |
| | ICE1_1 | FFDEDLQAAIDFFKLPPPLLSPVPSPPPMSSPHPGSLP |
| | | SSFAPETYFGEYTDSSDNDSVQLR |
| | ICE1_2 | PLISSSSPSSPASPVGQVSPFRETPVPPAMSPWPEDPR |
| | | RASPPDPSPSPSAASASERVVPSP |
| 2184 | ICE1_3 | SSPSSPASPVGQVSPFRETPVPPAMSPWPEDPRRASP |
| | | PDPSPSPSAASASERVVPSPLQFCA |
| | ICE1_4 | PASPVGQVSPFRETPVPPAMSPWPEDPRRASPPDPSP |
| | | SPSAASASERVVPSPLQFCAATPKH |
| | ICE1_5 | GQVSPFRETPVPPAMSPWPEDPRRASPPDPSPSPSAA |
| | | SASERVVPSPLQFCAATPKHALPVP |
| | ZBED4 | TSCLIRHMWRAHRAIVLQENGGTGIPPLYSTPPTLLP |
| | | SLLPPEGELSSVSSSPVKPVRESPS |
| | CAMSAP1_0 | ELKDAKTVLHQKSSRPPVPISNATKRSFLGSPAAGTL |
| | | AELQPPVQLPAEGCHRHYLHPEEPE |
| 2185 | CAMSAP1_1 | QPLVRRKMTGSRDLNRTFTPIPCSEFPMGIDPTETGP |
| | | LSVETAGEVCGGPLALGGFDPFPQG |
| | TBC1D17 | ELPHNVQEILGLAPPAEPHSPSPTASPLPLSPTRAPPT |
| | | PPPSTDTAPQPDSSLEILPEEEDE |
| | SLC12A9 | LGFYDDAPPQDHFLTDPAFSEPADSTREGSSPALSTL |
| | | FPPPRAPGSPRALNPQDYVATVADA |
| | DLG3 | ISHNSSLGYLGAVESKVSYPAPPQVPPTRYSPIPRHM |
| | | LAEEDFTREPRKIILHKGSTGLGFN |
| 2186 | SCARF1_0 | GTQCQQPCLPGTFGESCEQQCPHCRHGEACEPDTG |
| | | HCQRCDPGWLGPRCEDPCPTGTFGEDC |
| 2187 | SCARF1_1 | GTFGESCEQQCPHCRHGEACEPDTGHCQRCDPGWL |
| | | GPRCEDPCPTGTFGEDCGSTCPTCVQG |
| 2188 | SCARF1_2 | CPHCRHGEACEPDTGHCQRCDPGWLGPRCEDPCPT |
| | | GTFGEDCGSTCPTCVQGSCDTVTGDCV |
| | SCARF1_3 | GAQSGPEGREAEESTGPEEAEAPESFPAAASPGDSA |
| | | TGHRRPPLGGRTVAEHVEAIEGSVQE |
| | PRRX2_0 | MLASRSASLLKSYSQEAAIEQPVAPRPTALSPDYLS |
| | | WTASSPYSTVPPYSPGSSGPATPGVN |
| | PRRX2_1 | KSYSQEAAIEQPVAPRPTALSPDYLSWTASSPYSTVP |
| | | PYSPGSSGPATPGVNMANSIASLRL |
| 2189 | SGPP1 | RFQRLCGVEAPPRRSADRREDEKAEAPLAGDPRLR |
| | | GRQPGAPGGPQPPGSDRNQCPAKPDGG |
| | DOK2 | EEAISAQKNAAPATPQPQPATIPASLPRPDSPYSRPH |
| | | DSLPPPSPTTPVPAPRPRGQEGEYA |
| | ATF7 | GCGMVVGTASTMVTARPEQSQILIQHPDAPSPAQPQ |
| | | VSPAQPTPSTGGRRRRTVDEDPDERR |
| | UBQLN4 | QTEAPGLVPSLGSFGISRTPAPSAGSNAGSTPEAPTSS |
| | | PATPATSSPTGASSAQQQLMQQMI |
| 2190 | TET1 | AADGPGISQLGEVAPLPTLSAPVMEPLINSEPSTGVT |
| | | EPLTPHQPNHQPSFLTSPQDLASSP |
| | TANK | ACLPPGDHNALYVNSFPLLDPSDAPFPSLDSPGKAIR |
| | | GPQQPIWKPFPNQDSDSVVLSGTDS |
| 2191 | PDE12_0 | FPVCPKLSLEFGDPASSLFRWYKEAKPGAAEPEVGV |
| | | PSSLSPSSPSSSWTETDVEERVYTPS |
| | PDE12_1 | FGDPASSLFRWYKEAKPGAAEPEVGVPSSLSPSSPSS |
| | | SWTETDVEERVYTPSNADIGLRLKL |
| | RABL6 | ASPLAANGQSPSPGSQSPVVPAGAVSTGSSSPGTPQP |
| | | APQLPLNAAPPSSVPPVPPSEALPP |
| 2192 | FBRSL1 | GFAWEPFRGLELPRRAFPAAAPAPGSAALLEPPERP |
| | | YRDREPHGYSPERLRGELERARAPHL |
| | WNK1_0 | AVAPSKLLTSTTSTCLPPTNLPLGTVALPVTPVVTPG |
| | | QVSTPVSTTTSGVKPGTAPSKPPLT |
| 2193 | WNK1_1 | EGPVASPPFMDLEQAVLPAVIPKKEKPELSEPSHLNG |
| | | PSSDPEAAFLSRDVDDGSGSPHSPH |
| 2194 | TRIM65 | LRRNVALSGVLEVVRAGPARDPGPDPGPGPDPAAR |
| | | CPRHGRPLELFCRTEGRCVCSVCTVRE |
| 2195 | SEC24B | NSYDALEGGSYPDMLSSSASSPAPDPAPEPDPASAP |
| | | APASAPAPVVPQPSKMAKPFGYGYPT |
| | MORC2 | RSQADLKKLPLEVTTRPSTEEPVRRPQRPRSPPLPAV |
| | | IRNAPSRPPSLPTPRPASQPRKAPV |
| | MED12_0 | GVSSHSSHVISAQSTSTLPTTPAPQPPTSSTPSTPFSDL |
| | | LMCPQHRPLVFGLSCILQTILLC |
| | MED12_1 | IDPSSSVLFEDMEKPDFSLFSPTMPCEGKGSPSPEKP |
| | | DVEKEVKPPPKEKIEGTLGVLYDQP |
| 2196 | MED12_2 | QQRLLLYHTHLRPRPRAYYLEPLPLPPEDEEPPAPTL |
| | | LEPEKKAPEPPKTDKPGAAPPSTEE |
| | CDT1 | EKALSQLALRSAAPSSPGSPRPALPATPPATPPAASP |
| | | SALKGVSQDLLERIRAKEAQKQLAQ |
| 2197 | HCN3 | LVQHDRDMARGVRGRAPSTGAQLSGKPVLWEPLV |
| | | HAPLQAAAVTSNVAIALTHQRGPLPLSP |
| | CIPC | LQSWTVQPSFEVISAQPQLLFLHPPVPSPVSPCHTGE |
| | | KKSDSRNYLPILNSYTKIAPHPGKR |
| | RBPMS2 | ARDPYDLMGAALIPASPEAWAPYPLYTTELTPAISH |
| | | AAFTYPTATAAAAALHAQVRWYPSSD |
| 2198 | EPN3_0 | PSTHCSADPWDIPGFRPNTEASGSSWGPSADPWSPIP |
| | | SGTVLSRSQPWDLTPMLSSSEPWGR |
| | EPN3_1 | ASGSSWGPSADPWSPIPSGTVLSRSQPWDLTPMLSS |
| | | SEPWGRTPVLPAGPPTTDPWALNSPH |
| | FRAT1 | LRCALGDRGRVRGRAAPYCVAELATGPSALSPLPPQ |
| | | ADLDGPPGAGKQGIPQPLSGPCRRGW |
| | RERE_0 | PQDNESDSDSSAQQQMLQAQPPALQAPTGVTPAPSS |
| | | APPGTPQLPTPGPTPSATAVPPQGSP |
| | RERE_1 | SAQQQMLQAQPPALQAPTGVTPAPSSAPPGTPQLPT |
| | | PGPTPSATAVPPQGSPTASQAPNQPQ |
| | RERE_2 | MLQAQPPALQAPTGVTPAPSSAPPGTPQLPTPGPTPS |
| | | ATAVPPQGSPTASQAPNQPQAPTAP |
| | RERE_3 | TPAPSSAPPGTPQLPTPGPTPSATAVPPQGSPTASQAP |
| | | NQPQAPTAPVPHTHIQQAPALHPQ |
| | RERE_4 | QSALQSQQPPREQPLPPAPLAMPHIKPPPTTPIPQLPA |
| | | PQAHKHPPHLSGPSPFSMNANLPP |
| 2199 | RERE_5 | EKEREREREREREAERAAKASSSAHEGRLSDPQLSG |
| | | PGHMRPSFEPPPTTIAAVPPYIGPDT |
| | RERE_6 | RFPYPPGTLPNPLLGQPPHEHEMLRHPVFGTPYPRD |
| | | LPGAIPPPMSAAHQLQAMHAQSAELQ |
| | ETV5 | YGEKCLYNYCAYDRKPPSGFKPLTPPTTPLSPTHQN |
| | | PLFPPPQATLPTSGHAPAAGPVQGVG |
| | SYNJ2 | ASEEALSAVAPRDLEASSEPEPTPGAAKPETPQAPPL |
| | | LPRRPPPRVPAIKKPTLRRTGKPLS |
| | NBR1_0 | TAQDLLSFELLDINIVQELERVPHNTPVDVTPCMSPL |
| | | PHDSPLIEKPGLGQIEEENEGAGFK |
| | NBR1_1 | LDINIVQELERVPHNTPVDVTPCMSPLPHDSPLIEKP |
| | | GLGQIEEENEGAGFKALPDSMVSVK |
| | NBR1_2 | QTLETVPLIPEVVELPPSLPRSSPCVHHHGSPGVDLP |
| | | VTIPEVSSVPDQIRGEPRGSSGLVN |
| 2200 | NCKAP5L_0 | VLRALEETDPLLLCSPATPWRPPGQGPGSPEPINGEL |
| | | CGPPQPEPSPWAPCLLLGPGNLGGL |
| 2201 | NCKAP5L_1 | CSPATPWRPPGQGPGSPEPINGELCGPPQPEPSPWAP |
| | | CLLLGPGNLGGLLHWERLLGGLGGE |
| | NCKAP5L_2 | TSHFTACGSLTRTLDSGIGTFPPPDHGSSGTPSKNLP |
| | | KTKPPRLDPPPGVPPARPPPLTKVP |
| 2202 | NCKAP5L_3 | DSGIGTFPPPDHGSSGTPSKNLPKTKPPRLDPPPGVPP |
| | | ARPPPLTKVPRRAHTLEREVPGIE |
| 2203 | KLHL42 | LREARMTGTPVLVALGDFLGGPLAPHPYQGEPPSM |
| | | LRYEEMTERWFPLANNLPPDLVNVRGY |
| 2204 | PPP1R10 | KKVLSPTAAKPSPFEGKTSTEPSTAKPSSPEPAPPSEA |
| | | MDADRPGTPVPPVEVPELMDTASL |
| | KIFIC_0 | PFKSNPQHRESWPGMGSGEAPTPLQPPEEVTPHPAT |
| | | PARRPPSPRRSHHPRRNSLDGGGRSR |
| | KIF1C_1 | PQHRESWPGMGSGEAPTPLQPPEEVTPHPATPARRP |
| | | PSPRRSHHPRRNSLDGGGRSRGAGSA |
| | PHLDB1 | AMSVGSSYENTSPAFSPLSSPASSGSCASHSPSGQEP |
| | | GPSVPPLVPARSSSYHLALQPPQSR |
| 2205 | MRPS23 | FSRTRDLVRAGVLKEKPLWFDVYDAFPPLREPVFQR |
| | | PRVRYGKAKAPIQDIWYHEDRIRAKF |
| | EIF3F | APASSSDPAAAAAATAAPGQTPASAQAPAQTPAPA |
| | | LPGPALPGPFPGGRVVRLHPVILASIV |
| | UBE20 | EEKMEAVPDVERKEDKPEGQSPVKAEWPSETPVLC |
| | | QQCGGKPGVTFTSAKGEVFSVLEFAPS |
| 2206 | CHD6 | LRQQADYSLEVPGFGANFSDKPKQRRPRCKEPGKL |
| | | DVSSLSGEERVPAIPKEPGLRGFLPEN |
| 2207 | CEP192 | SLDVLPVKGPQGSPLLSRAARPPLDQLASEEPWTVL |
| | | PEHLILVAPSPCDMAKTGRFQIVNNS |
| | YLPM1_0 | KQQQYKHQMLHHQRDGPPGLVPMELESPPESPPVP |
| | | PGSYMPPSQSYMPPPQPPPSYYPPTSS |
| | YLPM1_1 | PSQSYMPPPQPPPSYYPPTSSQPYLPPAQPSPSQSPPS |
| | | QSYLAPTPSYSSSSSSSQSYLSHS |
| | YLPM1_2 | GHKKGPVVAKDTPEPVKEEVTVPATSQVPESPSSEE |
| | | PPLPPPNEEVPPPLPPEEPQSEDPEE |
| 2208 | YLPM1_3 | PVVAKDTPEPVKEEVTVPATSQVPESPSSEEPPLPPP |
| | | NEEVPPPLPPEEPQSEDPEEDARLK |
| | YLPM1_4 | SAGPPPVLPPPSLSSTAPPPVMPLPPLSSATPPPGIPPP |
| | | GVPQGIPPQLTAAPVPPASSSQS |
| | CDC42BPB | EPSVTVPLRSMSDPDQDFDKEPDSDSTKHSTPSNSSN |
| | | PSGPPSPNSPHRSQLPLEGLEQPAC |
| 2209 | MSL3 | TNRSQEELSPSPPLLNPSTPQSTESQPTTGEPATPKRR |
| | | KAEPEALQSLRRSTRHSANCDRLS |
| | MAP3K6 | AALGVLGPEVEKEAVSPRSEELSNEGDSQQSPGQQS |
| | | PLPVEPEQGPAPLMVQLSLLRAETDR |
| 2210 | CCDC34 | NAKHKPRPAAKSYGYANGKLTGFYSGNSYPEPAFY |
| | | NPIPWKPIHMPPPKEAKDLSGRKSKRP |
| | PKN3_0 | RGQDFLRASQMNLGMAAWGRLVMNLLPPCSSPSTI |
| | | SPPKGCPRTPTTLREASDPATPSNFLP |
| | PKN3_1 | LRASQMNLGMAAWGRLVMNLLPPCSSPSTISPPKG |
| | | CPRTPTTLREASDPATPSNFLPKKTPL |
| | PKN3_2 | LPKKTPLGEEMTPPPKPPRLYLPQEPTSEETPRTKRP |
| | | HMEPRTRRGPSPPASPTRKPPRLQD |
| 2211 | NUAK2_0 | TAHRPGKSNLKLPKGILKKKVSASAEGVQEDPPELS |
| | | PIPASPGQAAPLLPKKGILKKPRQRE |
| | NUAK2_1 | GKSNLKLPKGILKKKVSASAEGVQEDPPELSPIPASP |
| | | GQAAPLLPKKGILKKPRQRESGYYS |
| | NUAK2_2 | KLPKGILKKKVSASAEGVQEDPPELSPIPASPGQAAP |
| | | LLPKKGILKKPRQRESGYYSSPEPS |
| | CEP104 | YEQLELHSLLDAELMRRPFDLPLQPLARSGSPCHQK |
| | | PMPSLPQLEERGTENQFAEPFLQEKP |
| 2212 | DLGAP5 | RSATQAAKQVPRTVSSTTARKPVTRAANENEPEGK |
| | | VPSKGRPAKNVETKPDKGISCKVDSEE |
| | MAST3_0 | SSEDEGVGPGPAGPKRPVFILGEPDPPPAATPVMPKP |
| | | SSLSADTAALSHARLRSNSIGARHS |
| | MAST3_1 | LPGSPTHSLSPSPTTPCRSPAPDVPADTTASPPSASPS |
| | | SSSPASPAAAGHTRPSSLHGLAAK |
| | MAST3_2 | THSLSPSPTTPCRSPAPDVPADTTASPPSASPSSSSPA |
| | | SPAAAGHTRPSSLHGLAAKLGPPR |
| | MAST3_3 | RPSSLHGLAAKLGPPRPKTGRRKSTSSIPPSPLACPPI |
| | | SAPPPRSPSPLPGHPPAPARSPRL |
| | MAST3_4 | PRPKTGRRKSTSSIPPSPLACPPISAPPPRSPSPLPGHP |
| | | PAPARSPRLRRGQSADKLGTGER |
| | WNK4_0 | HRSWTAFSTSSSSPGTPLSPGNPFSPGTPISPGPIFPITS |
| | | PPCHPSPSPFSPISSQVSSNPS |
| | WNK4_1 | TPLSPGNPFSPGTPISPGPIFPITSPPCHPSPSPFSPISSQ |
| | | VSSNPSPHPTSSPLPFSSSTP |
| | WNK4_2 | GNPFSPGTPISPGPIFPITSPPCHPSPSPFSPISSQVSSNP |
| | | SPHPTSSPLPFSSSTPEFPVP |
| | WNK4_3 | SPSPFSPISSQVSSNPSPHPTSSPLPFSSSTPEFPVPLSQ |
| | | CPWSSLPTTSPPTFSPTCSQVT |
| | WNK4_4 | SAFSLAVMTVAQSLLSPSPGLLSQSPPAPPSPLPSLPL |
| | | PPPVAPGGQESPSPHTAEVESEAS |
| 2213 | PRRT3 | MNGADPISPQRVRGAVEAPGTPKSLIPGPSDPGPAV |
| | | NRTESPMGALQPDEAEEWPGRPQSHP |
| | CTTNBP2NL | NTANPRGDTSHSPTPGKVSSPLSPLSPGIKSPTIPRAE |
| | | RGNPPPIPPKKPGLTPSPSATTPL |
| 2214 | EEF1G | KPQAERKEEKKAAAPAPEEEMDECEQALAAEPKAK |
| | | DPFAHLPKSTFVLDEFKRKYSNEDTLS |
| 2215 | RBM20 | SPHGFSGQSKPDLTAGPMWPPPHNQPYELYDPEEPT |
| | | SDRTPPSFGGRLNNSKQGFIGAGRRA |
| | TAF3_0 | KVKDKGREDKMKAPAPPLVLPPKELALPLFSPATAS |
| | | RVPAMLPSLLPVLPEKLFEEKEKVKE |
| | TAF3_1 | RVGAGQDKIVISKVVPAPEAKPAPSQNRPKTPPPAP |
| | | APAPGPMLVSPAPVPLPLLAQAAAGP |
| | TAF3_2 | PAPEAKPAPSQNRPKTPPPAPAPAPGPMLVSPAPVPL |
| | | PLLAQAAAGPALLPSPGPAASGASA |
| 2216 | DHX34 | VVQVPGRLFPITVVYQPQEAEPTTSKSEKLDPRPFLR |
| | | VLESIDHKYPPEERGDLLVFLSGMA |
| | C1orf116_0 | LIPPPEAFRDTQPEQCREASLPEGPGQQGHTPQLHTP |
| | | SSSQEREQTPSEAMSQKAKETVSTR |
| | C1orf116_1 | EAFRDTQPEQCREASLPEGPGQQGHTPQLHTPSSSQ |
| | | EREQTPSEAMSQKAKETVSTRYTQPQ |
| | PHACTR4_0 | ITTKTPSDEREKSTCSMGSELLPMISPRSPSPPLPTHIP |
| | | PEPPRTPPFPAKTFQVVPEIEFP |
| 2217 | PHACTR4_1 | EKSTCSMGSELLPMISPRSPSPPLPTHIPPEPPRTPPFP |
| | | AKTFQVVPEIEFPPSLDLHQEIP |
| 2218 | PGM2 | TSVHGVGHSFVQSAFKAFDLVPPEAVPEQKDPDPEF |
| | | PTVKYPNPEEGKGVLTLSFALADKTK |
| | PARP10 | TLEGLDLDGEDWLPRELEEEGPQEQPEEEVTPGHEE |
| | | EEPVAPSTVAPRWLEEEAALQLALHR |
| 2219 | PAXIP1 | SPEKQERNLNWTPAEVPQLAAAKRRLPQGKEPGLIN |
| | | LCANVPPVPGNILPPEVRGNLMAAGQ |
| | SH3RF3_0 | GSCPIESEMQGAMGMEPLHRKAGSLDLNFTSPSRQA |
| | | PLSMAAIRPEPKLLPRERYRVVVSYP |
| 2220 | SH3RF3_1 | EPLHRKAGSLDLNFTSPSRQAPLSMAAIRPEPKLLPR |
| | | ERYRVVVSYPPQSEAEIELKEGDIV |
| | MED1_0 | RKKADTEGKSPSHSSSNRPFTPPTSTGGSKSPGSAGR |
| | | SQTPPGVATPPIPKITIQIPKGTVM |
| | MED1_1 | SNRPFTPPTSTGGSKSPGSAGRSQTPPGVATPPIPKITI |
| | | QIPKGTVMVGKPSSHSQYTSSGS |
| | MED1_2 | GLSSGSSSTKMKPQGKPSSLMNPSLSKPNISPSHSRP |
| | | PGGSDKLASPMKPVPGTPPSSKAKS |
| | MED1_3 | KPSSLMNPSLSKPNISPSHSRPPGGSDKLASPMKPVP |
| | | GTPPSSKAKSPISSGSGGSHMSGTS |
| 2221 | ELL_0 | PGYSEGDQQLLKRVLVRKLCQPQSTGSLLGDPAASS |
| | | PPGERGRSASPPQKRLQPPDFIDPLA |
| | ELL_1 | GDQQLLKRVLVRKLCQPQSTGSLLGDPAASSPPGER |
| | | GRSASPPQKRLQPPDFIDPLANKKPR |
| | CASP9 | LEDTGQDMLASFLRTNRQAAKLSKPTLENLTPVVL |
| | | RPEIRKPEVLRPETPRPVDIGSGGFGD |
| 2222 | HOXD4 | LYPRPDFGEQPFGGSGPGPGSALPARGHGQEPGGPG |
| | | GHYAAPGEPCPAPPAPPPAPLPGARA |
| | PPFIA3 | SRVSSSGLDSLGRYRSSCSLPPSLTTSTLASPSPPSSG |
| | | HSTPRLAPPSPAREGTDKANHVPK |
| 2223 | PHF12_0 | RPGTPTSSASTETPTSEQNDVDEDIIDVDEEPVAAEP |
| | | DYVQPQLRRPFELLIAAAMERNPTQ |
| 2224 | PHF12_1 | TSSASTETPTSEQNDVDEDIIDVDEEPVAAEPDYVQP |
| | | QLRRPFELLIAAAMERNPTQFQLPN |
| | GAK_0 | DLLSCLLGPPEAASQGPPEDLLSEDPLLLASPAPPLS |
| | | VQSTPRGGPPAAADPFGPLLPSSGN |
| | GAK_1 | EAASQGPPEDLLSEDPLLLASPAPPLSVQSTPRGGPP |
| | | AAADPFGPLLPSSGNNSQPCSNPDL |
| | GAK_2 | APCGSQASWTKSQNPDPFADLGDLSSGLQGSPAGFP |
| | | PGGFIPKTATTPKGSSSWQTSRPPAQ |
| 2225 | HAUS6 | KEFLGLSPFSLIKGWTPSVDLLPPMSPLSFDPASEEV |
| | | YAKSILCQYPASLPDAHKQHNQENG |
| 2226 | BARHL1 | ELLAEAGNYSALQRMFPSPYFYPQSLVSNLDPGAAL |
| | | YLYRGPSAPPPALQRPLVPRILIHGL |
| | RAPH1 | QAAPPTPTPPVPPAKKQPAFPASYIPPSPPTPPVPVPP |
| | | PTLPKQQSFCAKPPPSPLSPVPSV |
| | NOTO | SRVRPPRSGRSPAPRSPTGPNTPRAPGRFESPFSVEAI |
| | | LARPDPCAPAASQPSGSACVHPAF |
| | SNAI3 | PRASRAAIVPLKDSLNHLNLPPLLVLPTRWSPTLGPD |
| | | RHGAPEKLLGAERMPRAPGGFECFH |
| | CYP4F22 | IYGTHHNPTVWPDSKVYNPYRFDPDNPQQRSPLAY |
| | | VPFSAGPRNCIGQSFAMAELRVVVALT |
| | BCL9_0 | EMNRMIPGSQRHMEPGNNPIFPRIPVEGPLSPSRGDF |
| | | PKGIPPQMGPGRELEFGMVPSGMKG |
| | BCL9_1 | PGINPLKSPTMHQVQSPMLGSPSGNLKSPQTPSQLA |
| | | GMLAGPAAAASIKSPPVLGSAAASPV |
| | BCL9_2 | AGMLAGPAAAASIKSPPVLGSAAASPVHLKSPSLPA |
| | | PSPGWTSSPKPPLQSPGIPPNHKAPL |
| 2227 | UTF1_0 | RKRPRRRSPGSGRPQRARRPVPNAHAPAPSEPDATP |
| | | LPTARDRDADPTWTLRFSPSPPKSAD |
| | UTF1_1 | ATPLPTARDRDADPTWTLRFSPSPPKSADASPAPGSP |
| | | PAPAPTALATCIPEDRAPVRGPGSP |
| | MICALL2_0 | GGMAGVKRASEDSEEEPSGKKAPVQAAKLPSPAPA |
| | | RKPPLSPAQTNPVVQRRNEGAGGPPPK |
| | MICALL2_1 | KDSSKEQARNFLKQALSALEEAGAPAPGRPSPATAA |
| | | VPSSQPKTEAPQASPLAKPLQSSSPR |
| | MICALL2_2 | EEEKKPHLQGKPGRPLSPANVPALPGETVTSPVRLH |
| | | PDYLSPEEIQRQLQDIERRLDALELR |
| | POU6F1_0 | PQLLLNAQGQVIATLASSPLPPPVAVRKPSTPESPAK |
| | | SEVQPIQPTPTVPQPAVVIASPAPA |
| | POU6F1_1 | ASSPLPPPVAVRKPSTPESPAKSEVQPIQPTPTVPQPA |
| | | VVIASPAPAAKPSASAPIPITCSE |
| 2228 | PANK4 | GPAQRARSGTFDLLEMDRLERPLVDLPLLLDPPSYV |
| | | PDTVDLTDDALARKYWLTCFEEALDG |
| | MICAL3 | DAPSDLKAVHSPIRSQPVTLPEARTPVSPGSPQPQPP |
| | | VAASTPPPSPLPICSQPQPSTEATV |
| 2229 | ASHIL_0 | KEMPQLEGPPKRTLKIPASKVFSLQSKEEQEPPILQP |
| | | EIEIPSFKQGLSVSPFPKKRGRPKR |
| | ASHIL_1 | VFSLQSKEEQEPPILQPEIEIPSFKQGLSVSPFPKKRGR |
| | | PKRQMRSPVKMKPPVLSVAPFVA |
| | LCP2 | DEDDVHQRPLPQPALLPMSSNTFPSRSTKPSPMNPLP |
| | | SSHMPGAFSESNSSFPQSASLPPYF |
| | LHX5 | PLGALEPPLAGPHAADNPRFTDMISHPDTPSPEPGLP |
| | | GTLHPMPGEVFSGGPSPPFPMSGTS |
| 2230 | UBXN7 | LAKSRKSPHKDLGHRKEENRRPLTEPPVRTDPGTAT |
| | | NHQGLPAVDSEILEMPPEKADGVVEG |
| 2231 | SHROOM2 | RVLRATSFKRRDLDPNPGDLYPESLEHRMGDPDTVP |
| | | HFWEAGLAQPPSSTSGGPHPPRIGGR |
| 2232 | FLAD1 | EKTRVFLEGSTRTPALPHCLFWLLQVPSTQDPLFPG |
| | | YGPQCPVDLAGPPCLRPLFGGLGGYW |
| | PRICKLE3 | EYAWVPPGLKPEQVYQFFSCLPEDKVPYVNSPGEK |
| | | YRIKQLLHQLPPHDSEAQYCTALEEEE |
| | MAP3K1_0 | NSPSGRTVKSESPGVRRKRVSPVPFQSGRITPPRRAP |
| | | SPDGFSPYSPEETNRRVNKVMRARL |
| | MAP3K1_1 | MVQTKGRPHSQCLNSSPLSHHSQLMFPALSTPSSSTP |
| | | SVPAGTATDVSKHRLQGFIPCRIPS |
| | DYNCILI1 | KLQSLLAKQPPTAAGRPVDASPRVPGGSPRTPNRSV |
| | | SSNVASVSPIPAGSKKIDPNMKAGAT |
| | ZFHX3 | FDNTPLQALNLPTAYPALQGIPPVLLPGLNSPSLPGF |
| | | TPSNTALTSPKPNLMGLPSTTVPSP |
| | CCNO | LHPLNPCPLPGDSGICDLFESPSSGSDGAESPSAARG |
| | | GSPLPGPAQPVAQLDLQTFRDYGQS |
| 2233 | VEZF1 | TAFLFQAHEASHHQQQAAQNSLLPLLSSAVEPPDQK |
| | | PLLPIPITQKPQGAPETLKDAIGIKK |
| | WAC_0 | SHSCTTPSTSSASGLNPTSAPPTSASAVPVSPVPQSPI |
| | | PPLLQDPNLLRQLLPALQATLQLN |
| | WAC_1 | SPRISTPQTNTVPIKPLISTPPVSSQPKVSTPVVKQGP |
| | | VSQSATQQPVTADKQQGHEPVSPR |
| 2234 | SPAG17_0 | NEKPVLEAMPTSEAPQPAVPAPGKKKAQYEEPQAP |
| | | PPVTSVITTEVDMRYYNYLLNPIREEF |
| 2235 | SPAG17_1 | SLKKKSPYKEKSKEEQVKIQEVTEESPHQPEPKITYP |
| | | FHGYNMGNIPTQISGSNYYLYPSDG |
| | SCML2 | LPTQQVRRSSRIKPPGPTAVPKRSSSVKNITPRKKGP |
| | | NSGKKEKPLPVICSTSAASLKSLTR |
| 2236 | ZNF512B_0 | FPCTHCGKTYRSKAGHDYHVRSEHTAPPPEEPTDKS |
| | | PEAEDPLGVERTPSGRVRRTSAQVAV |
| | ZNF512B_1 | CGKTYRSKAGHDYHVRSEHTAPPPEEPTDKSPEAED |
| | | PLGVERTPSGRVRRTSAQVAVFHLQE |
| 2237 | ZNF512B_2 | RSKAGHDYHVRSEHTAPPPEEPTDKSPEAEDPLGVE |
| | | RTPSGRVRRTSAQVAVFHLQEIAEDE |
| | SCYL1_0 | AVTGVSSLTSKLIRSHPTTAPTETNIPQRPTPEGVPAP |
| | | APTPVPATPTTSGHWETQEEDKDT |
| | SCYL1_1 | KLIRSHPTTAPTETNIPQRPTPEGVPAPAPTPVPATPT |
| | | TSGHWETQEEDKDTAEDSSTADRW |
| | TRIOBP_0 | ISRASSTQQETSRASSTQEDTPRASSTQEDTPRASST |
| | | QWNTPRASSPSRSTQLDNPRTSSTQ |
| 2238 | TRIOBP_1 | SSTQQDNPQTSFPTCTPQRENPRTPCVQQDDPRASSP |
| | | NRTTQRENSRTSCAQRDNPKASRTS |
| | TRIOBP_2 | AAYGAPLTSPEPSQPPCAVCIGHRDAPRASSPPRYLQ |
| | | HDPFPFFPEPRAPESEPPHHEPPYI |
| 2239 | TRIOBP_3 | SPEPSQPPCAVCIGHRDAPRASSPPRYLQHDPFPFFPE |
| | | PRAPESEPPHHEPPYIPPAVCIGH |
| 2240 | TRIOBP_4 | RASSPPRYLQHDPFPFFPEPRAPESEPPHHEPPYIPPA |
| | | VCIGHRDAPRASSPPRHTQFDPFP |
| | TRIOBP_5 | RAPESEPPHHEPPYIPPAVCIGHRDAPRASSPPRHTQF |
| | | DPFPFLPDTSDAEHQCQSPQHEPL |
| | TRIOBP_6 | AEHQCQSPQHEPLQLPAPVCIGYRDAPRASSPPRQA |
| | | PEPSLLFQDLPRASTESLVPSMDSLH |
| | TRIOBP_7 | SLVPSMDSLHECPHIPTPVCIGHRDAPSFSSPPRQAPE |
| | | PSLFFQDPPGTSMESLAPSTDSLH |
| | TRIOBP_8 | SLAPSTDSLHGSPVLIPQVCIGHRDAPRASSPPRHPPS |
| | | DLAFLAPSPSPGSSGGSRGSAPPG |
| 2241 | SIPA1L3 | SPQKGLQRTLSDESLCSGRREPSFASPAGLEPGLPSD |
| | | VLFTSTCAFPSSTLPARRQHQHPHP |
| | NELFA | LNNEPALPSTSYLPSTPSVVPASSYIPSSETPPAPSSRE |
| | | ASRPPEEPSAPSPTLPAQFKQRA |
| 2242 | BCR_0 | QAPDGASEPRASASRPQPAPADGADPPPAEEPEARP |
| | | DGEGSPGKARPGTARRPGAAASGERD |
| | BCR_1 | ASASRPQPAPADGADPPPAEEPEARPDGEGSPGKAR |
| | | PGTARRPGAAASGERDDRGPPASVAA |
| 2243 | EPS15_0 | DPFRSATSSSVSNVVITKNVFEETSVKSEDEPPALPP |
| | | KIGTPTRPCPLPPGKRSINKLDSPD |
| | EPS15_1 | VSNVVITKNVFEETSVKSEDEPPALPPKIGTPTRPCPL |
| | | PPGKRSINKLDSPDPFKLNDPFQP |
| 2244 | EPS15_2 | KIGTPTRPCPLPPGKRSINKLDSPDPFKLNDPFQPFPG |
| | | NDSPKEKDPEIFCDPFTSATTTTN |
| | EPS15_3 | LPPGKRSINKLDSPDPFKLNDPFQPFPGNDSPKEKDP |
| | | EIFCDPFTSATTTTNKEADPSNFAN |
| 2245 | EPS15_4 | RSINKLDSPDPFKLNDPFQPFPGNDSPKEKDPEIFCDP |
| | | FTSATTTTNKEADPSNFANFSAYP |
| | JCAD | HSQQQSPTEKAGASGQPPSGPPGTGNEYGVSPRLPQ |
| | | GLPAHPRPVTAYDGFVQYIPFDDPRL |
| | EP400 | QAAQLAGQRQSQQQYDPSTGPPVQNAASLHTPLPQ |
| | | LPGRLPPAGVPTAALSSALQFAQQPQV |
| | SGIP1 | ESAFDEQKTEVLLDQPEIWGSGQPINPSMESPKLTRP |
| | | FPTGTPPPLPPKNVPATPPRTGSPL |
| | FBXO42 | GQCVVVFSQAPSGRAPLSPSLNSRPSPISATPPALVPE |
| | | TREYRSQSPVRSMDEAPCVNGRWG |
| 2246 | ZNF574_0 | GVGGVPLPTTPVPPEEPVIGFPEPAPAETGEPEAPEPP |
| | | VSEETSAGPAAPGTYRCLLCSREF |
| 2247 | ZNF574_1 | PLPTTPVPPEEPVIGFPEPAPAETGEPEAPEPPVSEETS |
| | | AGPAAPGTYRCLLCSREFGKALQ |
| | SP2_0 | SPLALLAATCSKIGPPAVEAAVTPPAPPQPTPRKLVPI |
| | | KPAPLPLSPGKNSFGILSSKGNIL |
| | SP2_1 | PAVEAAVTPPAPPQPTPRKLVPIKPAPLPLSPGKNSF |
| | | GILSSKGNILQIQGSQLSASYPGGQ |
| 2248 | COL4A1_0 | GYGPAGPIGDKGQAGFPGGPGSPGLPGPKGEPGKIV |
| | | PLPGPPGAEGLPGSPGFPGPQGDRGF |
| | COL4A1_1 | PGSPGLPGPKGEPGKIVPLPGPPGAEGLPGSPGFPGP |
| | | QGDRGFPGTPGRPGLPGEKGAVGQP |
| | COL4A1_2 | IVPLPGPPGAEGLPGSPGFPGPQGDRGFPGTPGRPGL |
| | | PGEKGAVGQPGIGFPGPPGPKGVDG |
| 2249 | COL4A1_3 | LPGLDGIPGVKGEAGLPGTPGPTGPAGQKGEPGSDG |
| | | IPGSAGEKGEPGLPGRGFPGFPGAKG |
| 2250 | ZC3H12C | YGYRQTYSLPDNSTQPCYEQFTFQSLPEQQEPAWRI |
| | | PYCGMPQDPPRYQDNREKIYINLCNI |
| | CHAF1B_0 | VLNMRTPDTAKKTKSQTHRGSSPGPRPVEGTPASRT |
| | | QDPSSPGTTPPQARQAPAPTVIRDPP |
| | CHAF1B_1 | KKTKSQTHRGSSPGPRPVEGTPASRTQDPSSPGTTPP |
| | | QARQAPAPTVIRDPPSITPAVKSPL |
| 2251 | CHAF1B_2 | GTPASRTQDPSSPGTTPPQARQAPAPTVIRDPPSITPA |
| | | VKSPLPGPSEEKTLQPSSQNTKAH |
| | C6orf132_0 | RSPAEPKGSALGPNPEPHLTFPRSFKVPPPTPVRTSSI |
| | | PVQEAQEAPRKEEGATKKAPSRLP |
| | C6orf132_1 | KNLPPQSTTLLPTTSLQPKAMLGPAIPPKATPEPAIPP |
| | | KATLWPATPPKATLGPATPLKATS |
| | C6orf132_2 | LQPKAMLGPAIPPKATPEPAIPPKATLWPATPPKATL |
| | | GPATPLKATSGPTTPLKATSGPAIA |
| | PCGF2_0 | SGASECESVSDKAPSPATLPATSSSLPSPATPSHGSPS |
| | | SHGPPATHPTSPTPPSTASGATTA |
| | PCGF2_1 | CESVSDKAPSPATLPATSSSLPSPATPSHGSPSSHGPP |
| | | ATHPTSPTPPSTASGATTAANGGS |
| | PCGF2_2 | ATSSSLPSPATPSHGSPSSHGPPATHPTSPTPPSTASG |
| | | ATTAANGGSLNCLQTPSSTSRGRK |
| | SRCAP_0 | GPALLTSVTPPLAPVVPAAPGPPSLAPSGASPSASAL |
| | | TLGLATAPSLSSSQTPGHPLLLAPT |
| | SRCAP_1 | GAASTLVPGVSETSASPGSPSVRSMSGPESSPPIGGP |
| | | CEAAPSSSLPTPPQQPFIARRHIEL |
| 2252 | SRCAP_2 | RGVDEAPSSTLKGKTNGADPVPGPETLIVADPVLEP |
| | | QLIPGPQPLGPQPVHRPNPLLSPVEK |
| | SRCAP_3 | IVADPVLEPQLIPGPQPLGPQPVHRPNPLLSPVEKRR |
| | | RGRPPKARDLPIPGTISSAGDGNSE |
| | SYNPO2_0 | RMVPMNRTAKPFPGSVNQPATPFSPTRNMTSPIADF |
| | | PAPPPYSAVTPPPDAFSRGVSSPIAG |
| | SYNPO2_1 | MKQALPPRPVNAASPTNVQASSVYSVPAYTSPPSFF |
| | | AEASSPVSASPVPVGIPTSPKQESAS |
| | SYNPO2_2 | NAASPTNVQASSVYSVPAYTSPPSFFAEASSPVSASP |
| | | VPVGIPTSPKQESASSSYFVAPRPK |
| | CHRNA10_0 | ARALLLGHLARGLCVRERGEPCGQSRPPELSPSPQSP |
| | | EGGAGPPAGPCHEPRCLCRQEALLH |
| | CHRNA10_1 | LGHLARGLCVRERGEPCGQSRPPELSPSPQSPEGGA |
| | | GPPAGPCHEPRCLCRQEALLHHVATI |
| 2253 | CNKSR1 | FDLSSNPSPGPSPAWTDSASLGPEPLPIPPEPPAILPAG |
| | | VAGTPGLPESPDKSPVGRKKSKG |
| 2254 | ZNF174 | AKGAKPCAVSAGRSKGNGLQNPEPRGANMSEPRLS |
| | | RRQVSSPNAQKPFAHYQRHCRVEYISS |
| 2255 | CCNK | PKIETTHPPLPPAHPPPDRKPPLAAALGEAEPPGPVD |
| | | ATDLPKVQIPPPAHPAPVHQPPPLP |
| | KIAA1522_0 | LPRPPTTGGSEGAGAAPCPPNPANSWVPGLSPGGSR |
| | | RPPRSPERTLSPSSGYSSQSGTPTLP |
| | KIAA1522_1 | APSDRSGPQILTPLGDRFVIPPHPKVPAPFSPPPSKPR |
| | | SPNPAAPALAAPAVVPGPVSTTDA |
| | KIAA1522_2 | MADFPPPEEAFFSVASPEPAGPSGSPELVSSPAASSSS |
| | | ATALQIQPPGSPDPPPAPPAPAPA |
| | KIAA1522_3 | SPETQADLQRNLVAELRSISEQRPPQAPKKSPKAPPP |
| | | VARKPSVGVPPPASPSYPRAEPLTA |
| | KIAA1522_4 | EQRPPQAPKKSPKAPPPVARKPSVGVPPPASPSYPRA |
| | | EPLTAPPTNGLPHTQDRTKRELAEN |
| 2256 | KIAA1522_5 | PKKSPKAPPPVARKPSVGVPPPASPSYPRAEPLTAPP |
| | | TNGLPHTQDRTKRELAENGGVLQLV |
| | BCLAF1_0 | DEFNKSSATSGDIWPGLSAYDNSPRSPHSPSPIATPPS |
| | | QSSSCSDAPMLSTVHSAKNTPSQH |
| | BCLAF1_1 | KNTPSQHSHSIQHSPERSGSGSVGNGSSRYSPSQNSPI |
| | | HHIPSRRSPAKTIAPQNAPRDESR |
| | BCLAF1_2 | QHSHSIQHSPERSGSGSVGNGSSRYSPSQNSPIHHIPS |
| | | RRSPAKTIAPQNAPRDESRGRSSF |
| | BCLAF1_3 | ERSGSGSVGNGSSRYSPSQNSPIHHIPSRRSPAKTIAP |
| | | QNAPRDESRGRSSFYPDGGDQETA |
| | JPH1 | DYVKQRFQEGVDAKENPEEKVPEKPPTPKESPHFYR |
| | | KGTTPPRSPEASPKHSHSPASSPKPL |
| | NCOA2 | YALKMNSPSQSSPGMNPGQPTSMLSPRHRMSPGVA |
| | | GSPRIPPSQFSPAGSLHSPVGVCSSTG |
| | RBSN | AVAGNPFIQPDSPAPNPFSEEDEHPQQRLSSPLVPGN |
| | | PFEEPTCINPFEMDSDSGPEAEEPI |
| | PDLIM5 | LDSPTSGRPGVTSLTAAAAFKPVGSTGVIKSPSWQR |
| | | PNQGVPSTGRISNSATYSGSVAPANS |
| 2257 | ZNF219_0 | LTAHGAPERPLAATSAAPPPQPQPQPPPQPEPRSVPQ |
| | | PEPEPEPEREATPTPAPAAPEEPPA |
| 2258 | ZNF219_1 | LAATSAAPPPQPQPQPPPQPEPRSVPQPEPEPEPEREA |
| | | TPTPAPAAPEEPPAPPEFRCQVCG |
| | HOXC4 | RGHGPAQAGHHHPEKSQSLCEPAPLSGASASPSPAP |
| | | PACSQPAPDHPSSAASKQPIVYPWMK |
| | PPP1R13L_0 | GSPRKAATDGADTPFGRSESAPTLHPYSPLSPKGRPS |
| | | SPRTPLYLQPDAYGSLDRATSPRPR |
| | PPP1R13L_1 | LQPQPQPQPQPQSQPQPQLPPQPQTQPQTPTPAPQHP |
| | | QQTWPPVNEGPPKPPTELEPEPEIE |
| 2259 | PPP1R13L_2 | QPQTPTPAPQHPQQTWPPVNEGPPKPPTELEPEPEIE |
| | | GLLTPVLEAGDVDEGPVARPLSPTR |
| | PPP1R13L_3 | HPQQTWPPVNEGPPKPPTELEPEPEIEGLLTPVLEAG |
| | | DVDEGPVARPLSPTRLQPALPPEAQ |
| | FAM184A | NRFVSVPNLSALESGGVGNGHPNRLDPIPNSPVHDIE |
| | | FNSSKPLPQPVPPKGPKTFLSPAQS |
| 2260 | CYFIP2 | FWELNFDFLPNYCYNGSTNRFVRTAIPFTQEPQRDK |
| | | PANVQPYYLYGSKPLNIAYSHIYSSY |
| | SCRIB | YRALAAVPSAGSVQRVPSGAAGGKMAESPCSPSGQ |
| | | QPPSPPSPDELPANVKQAYRAFAAVPT |
| | ARHGEF17_0 | RGAWPSVTEMRKLFGGPGSRRPSADSESPGTPSPDG |
| | | AAWEPPARESRQPPTPPPRTCFPLAG |
| | ARHGEF17_1 | IAVCSARILCIGAVPGLQPRCHREPPPSLRSPPETAPE |
| | | PAGPELDVEAAADEEAATLAEPGP |
| | ATN1_0 | SDSSSGLSQGPARPYHPPPLFPPSPQPPDSTPRQPEAS |
| | | FEPHPSVTPTGYHAPMEPPTSRMF |
| 2261 | ATN1_1 | PQPPDSTPRQPEASFEPHPSVTPTGYHAPMEPPTSRM |
| | | FQAPPGAPPPHPQLYPGGTGGVLSG |
| | ATN1_2 | ASGPPLSATQIKQEPAEEYETPESPVPPARSPSPPPKV |
| | | VDVPSHASQSARFNKHLDRGFNSC |
| 2262 | ARMH4_0 | LTTNPKTEKFEADTDHRTTSFPGAESTAGSEPGSLTP |
| | | DKEKPSQMTADNTQAAATKQPLETS |
| | ARMH4_1 | KTEKFEADTDHRTTSFPGAESTAGSEPGSLTPDKEKP |
| | | SQMTADNTQAAATKQPLETSEYTLS |
| 2263 | HOMEZ | PPPVPAPEQVGIGIGPPTLSKPTQTKGLKVEPEEPSQ |
| | | MPPLPQSHQKLKESLMTPGSGAFPY |
| 2264 | TRIL_0 | PSPSVAAAAGPAPQSLDLHKKPQRGRPTRADPALAE |
| | | PTPTASPGSAPSPAGDPWQRATKHRL |
| 2265 | TRIL_1 | AAAAGPAPQSLDLHKKPQRGRPTRADPALAEPTPT |
| | | ASPGSAPSPAGDPWQRATKHRLGTEHQ |
| 2266 | TSC22D4_0 | TDYEGPGSPGASDPPTPQPPTGPPPRLPNGEPSPDPG |
| | | GKGTPRNGSPPPGAPSSRFRVVKLP |
| | TSC22D4_1 | YEGPGSPGASDPPTPQPPTGPPPRLPNGEPSPDPGGK |
| | | GTPRNGSPPPGAPSSRFRVVKLPHG |
| | TSC22D4_2 | ASDPPTPQPPTGPPPRLPNGEPSPDPGGKGTPRNGSP |
| | | PPGAPSSRFRVVKLPHGLGEPYRRG |
| | TSC22D4_3 | TPQPPTGPPPRLPNGEPSPDPGGKGTPRNGSPPPGAP |
| | | SSRFRVVKLPHGLGEPYRRGRWTCV |
| 2267 | ADGRA2 | GLTCTAFQRREGGVPGTRPGSPGQNPPPEPEPPADQ |
| | | QLRFRCTTGRPNVSLSSFHIKNSVAL |
| | BCAR3_0 | HGTLPRKKKGPPPIRSCDDFSHMGTLPHSKSPRQNSP |
| | | VTQDGIQESPWQDRHGETFTFRDPH |
| | BCAR3_1 | RKKKGPPPIRSCDDFSHMGTLPHSKSPRQNSPVTQD |
| | | GIQESPWQDRHGETFTFRDPHLLDPT |
| | SMAD5_0 | LLVQFRNLSHNEPHMPQNATFPDSFHQPNNTPFPLS |
| | | PNSPYPPSPASSTYPNSPASSGPGSP |
| | SMAD5_1 | RNLSHNEPHMPQNATFPDSFHQPNNTPFPLSPNSPYP |
| | | PSPASSTYPNSPASSGPGSPFQLPA |
| 2268 | RIPOR1 | SYTQADPMAPRTPHPSPAHSSRKPLTSPAPDPSESTV |
| | | QSLSPTPSPPTPAPQHSDLCLAMAV |
| | ARGFX | KKQQQQQSAKQRNQILPSKKNVPTSPRTSPSPYAFS |
| | | PVISDFYSSLPSQPLDPSNWAWNSTF |
| 2269 | NFATC2 | AGLLVEQPPLAGVAASPRFTLPVPGFEGYREPLCLSP |
| | | ASSGSSASFISDTFSPYTSPCVSPN |
| | SYNPO_0 | VLRPEPTKQPPYQLRPSLFVLSPIKEPAKVSPRAASP |
| | | AKPSSLDLVPNLPKGALPPSPALPR |
| | SYNPO_1 | PTKQPPYQLRPSLFVLSPIKEPAKVSPRAASPAKPSSL |
| | | DLVPNLPKGALPPSPALPRPSRSS |
| | CHAMP1_0 | PEHQKIPCNSAEPKSIPALSMETQKLGSVLSPESPKPT |
| | | PLTPLEPQKPGSVVSPELQTPLPS |
| | CHAMP1_1 | SPEPPKSVPVCESQKLAPVPSPEPQKPAPVSPESVKA |
| | | TLSNPKPQKQSHFPETLGPPSASSP |
| | PLEKHA7_0 | KNPERKTVPLFPHPPVPSLSTSESKPPPQPSPPTSPVR |
| | | TPLEVRLFPQLQTYVPYRPHPPQL |
| | PLEKHA7_1 | LEVRLFPQLQTYVPYRPHPPQLRKVTSPLQSPTKAK |
| | | PKVEDEAPPRPPLPELYSPEDQPPAV |
| | PLEKHA7_2 | KVTSPLQSPTKAKPKVEDEAPPRPPLPELYSPEDQPP |
| | | AVPPLPREATIIRHTSVRGLKRQSD |
| | SEC24C | SQPNHVSSPPQALPPGTQMTGPLGPLPPMHSPQQPG |
| | | YQPQQNGSFGPARGPQSNYGGPYPAA |
| | ARHGEF10 | QAPSAPETGGAGASEAPAPTGGEDGAGAETTPVAEP |
| | | TKLVLPMKVNPYSVIDITPFQEDQPP |
| | EVL | SEAGRKPWERSNSVEKPVSSILSRTPSVAKSPEAKSP |
| | | LQSQPHSRMKPAGSVNDMALDAFDL |
| | PLIN1_0 | AERRASGAPSAGPEPAPRLAQPRRSLRSAQSPGAPP |
| | | GPGLEDEVATPAAPRPGFPAVPREKP |
| | PLIN1_1 | APRLAQPRRSLRSAQSPGAPPGPGLEDEVATPAAPR |
| | | PGFPAVPREKPKRRVSDSFFRPSVME |
| | THRAP3 | WPDATYGTGSASRASAVSELSPRERSPALKSPLQSV |
| | | VVRRRSPRPSPVPKPSPPLSSTSQMG |
| | PLEKHG4 | VLSEGPGPSGVESLLCPMSSHLSLAQGESDTPGVGL |
| | | VGDPGPSRAMPSGLSPGALDSDPVGL |
| | FNBP4 | DSTLANFLAEIDAITAPQPAAPVGASAPPPTPPRPEPK |
| | | EAATSTLSSSTSNGTDSTQTSGWQ |
| | RREB1_0 | EEAGSSEQPSPCPAPGPSLPVTLGPSGILESPMAPAPA |
| | | ATPEPPAQPLQGPVQLAVPIYSSA |
| | RREB1_1 | ASATKDCSHREEKVTAGWPSEPGQGDLNPESPAAL |
| | | GQDLLEPRSKRPAHPILATADGASQLV |
| | IRX2_0 | LKQPSLGPGCGPPGLPAAAAPASTGAPPGGSPYPAS |
| | | PLLGRPLYYTSPFYGNYTNYGNLNAA |
| | IRX2_1 | LGPGCGPPGLPAAAAPASTGAPPGGSPYPASPLLGR |
| | | PLYYTSPFYGNYTNYGNLNAALQGQG |
| 2270 | KATNAL1 | QVKSIVSTLESFKIDKPPDFPVSCQDEPFRDPAVWPP |
| | | PVPAEHRAPPQIRRPNREVRPLRKE |
| 2271 | GAB3 | GLGPHCSPDDYIPMNSGSISSPLPELPANLEPPPVNR |
| | | DLKPQRKSRPPPLDLRNLSIIREHA |
| | PDHX | DALKLVQLKQTGKITESRPTPAPTATPTAPSPLQATA |
| | | GPSYPRPVIPPVSTPGQPNAVGTFT |
| 2272 | CDK12 | EKEQRTRHLLTDLPLPPELPGGDLSPPDSPEPKAITPP |
| | | QQPYKKRPKICCPRYGERRQTESD |
| | SALL2 | PFSAGGVGRSHKPTPAPSPALPGSTDQLIASPHLAFP |
| | | STTGLLAAQCLGAARGLEATASPGL |
| | AUTS2_0 | PLSTQPPQGPPEAQLQPAPQPQVQRPPRPQSPTQLLH |
| | | QNLPPVQAHPSAQSLSQPLSAYNSS |
| 2273 | AUTS2_1 | AKQLARVPSPYVRTPVVESARPNSTSSREAEPRKGE |
| | | PAYENPKKSSEVKVKEERKEDHDLPP |
| 2274 | AUTS2_2 | RVPSPYVRTPVVESARPNSTSSREAEPRKGEPAYENP |
| | | KKSSEVKVKEERKEDHDLPPEAPQT |
| | FOSL1_0 | MSGSQELQWMVQPHFLGPSSYPRPLTYPQYSPPQPR |
| | | PGVIRALGPPPGVRRRPCEQISPEEE |
| | FOSL1_1 | RPVPCISLSPGPVLEPEALHTPTLMTTPSLTPFTPSLV |
| | | FTYPSTPEPCASAHRKSSSSSGDP |
| 2275 | SOWAHB_0 | ARHPQVPEARDQGPIRAWSVLPDNFLQLPLEPGSTE |
| | | PNSEPPDPCLSSHSLFPVVPDESWES |
| 2276 | SOWAHB_1 | VPEARDQGPIRAWSVLPDNFLQLPLEPGSTEPNSEPP |
| | | DPCLSSHSLFPVVPDESWESWAGNP |
| | BSX | KPLREVAPDHFASSLASRVPLLDYGYPLMPTPTLLA |
| | | PHAHHPLHKGDHHHPYFLTTSGMPVP |
| 2277 | PRRC2A_0 | SLKAENKGNDPNVSLVPKDGTGWASKQEQSDPKSS |
| | | DASTAQPPESQPLPASQTPASNQPKRP |
| 2278 | PRRC2A_1 | EADGKKGNSPNSEPPTPKTAWAETSRPPETEPGPPA |
| | | PKPPLPPPHRGPAGNWGPPGDYPDRG |
| 2279 | PRRC2A_2 | PSTPAPPPAVPKELPAPPAPPPASAPTPETEPEEPAQA |
| | | PPAQSTPTPGVAAAPTLVSGGGST |
| 2280 | PRRC2A_3 | PAPPPAVPKELPAPPAPPPASAPTPETEPEEPAQAPPA |
| | | QSTPTPGVAAAPTLVSGGGSTSST |
| 2281 | PRRC2A_4 | VSGGGSTSSTSSGSFEASPVEPQLPSKEGPEPPEEVPP |
| | | PTTPPVPKVEPKGDGIGPTRQPPS |
| | PRRC2A_5 | VSSGPCSQRSSPDGGLKGAAEGPPKRPGGSSPLNAV |
| | | PCEGPPGSEPPRRPPPAPHDGDRKEL |
| | PRRC2A_6 | PLSLLPVGPALQPPSLAVRPPPAPATRVLPSPARPFPA |
| | | SLGRAELHPVELKPFQDYQKLSSN |
| | DBNDD1 | AEVFADSDDENLNTESPAGLHPLPRAGYLRSPSWTR |
| | | TRAEQSHEKQPLGDPERQATVLDTFL |
| | TENT2 | YSLVLMVLHYLQTLPEPILPSLQKIYPESFSPAIQLHL |
| | | VHQAPCNVPPYLSKNESNLGDLLL |
| | PACS2_0 | VVKVGIVEPSSATSGDSDDAAPSGSGTLSSTPPSASP |
| | | AAKEASPTPPSSPSVSGGLSSPSQG |
| | PACS2_1 | IVEPSSATSGDSDDAAPSGSGTLSSTPPSASPAAKEA |
| | | SPTPPSSPSVSGGLSSPSQGVGAEL |
| 2282 | HES7 | AHDASPAARAQLFSALHGYLRPKPPRPKPVDPRPPA |
| | | PRPSLDPAAPALGPALHQRPPVHQGH |
| | GRAMD1A | RASSDADHGAEEDKEEQVDSQPDASSSQTVTPVAEP |
| | | PSTEPTQPDGPTTLGPLDLLPSEELL |
| 2283 | TAF1C_0 | CSWRDALTLPEAQPQNSENGALHVTKDLLWEPATP |
| | | GPLPMLPPLIDPWDPGLTARDLLFRGG |
| 2284 | TAF1C_1 | NSENGALHVTKDLLWEPATPGPLPMLPPLIDPWDPG |
| | | LTARDLLFRGGCRYRKRPRVVLDVTE |
| 2285 | DENND4C | YPEEDYESFPLSESDVPLFCLPMGATIECWDPETKYP |
| | | LPVFSTFVLTGSSAKKVYGAAIQFY |
| 2286 | SHROOM1_0 | ALARGTGQPGSRPTWPSQCLEELVQELARLDPSLCD |
| | | PLASQPSPEPPLGLLDGLIPLAEVRA |
| 2287 | SHROOM1_1 | TGQPGSRPTWPSQCLEELVQELARLDPSLCDPLASQ |
| | | PSPEPPLGLLDGLIPLAEVRAAMRPA |
| | CHD4_0 | KVQEFEHVNGRWSMPELAEVEENKKMSQPGSPSPK |
| | | TPTPSTPGDTQPNTPAPVPPAEDGIKI |
| | CHD4_1 | EHVNGRWSMPELAEVEENKKMSQPGSPSPKTPTPST |
| | | PGDTQPNTPAPVPPAEDGIKIEENSL |
| | CHD4_2 | VNGRWSMPELAEVEENKKMSQPGSPSPKTPTPSTPG |
| | | DTQPNTPAPVPPAEDGIKIEENSLKE |
| | CHD4_3 | RWSMPELAEVEENKKMSQPGSPSPKTPTPSTPGDTQ |
| | | PNTPAPVPPAEDGIKIEENSLKEEES |
| | FAM168A | ASSAAFRYTAGTPYKVPPTQSNTAPPPYSPSPNPYQT |
| | | AMYPIRSAYPQQNLYAQGAYYTQPV |
| | HOXD12 | FYFSNLRPNGGQLAALPPISYPRGALPWAATPASCA |
| | | PAQPAGATAFGGFSQPYLAGSGPLGL |
| | CEP85 | PHSNSSGVLPLGLQPAPGLSKPLPSQVWQPSPDTWH |
| | | PREQSCELSTCRQQLELIRLQMEQMQ |
| | EIF4G1 | DDRSQGAIIADRPGLPGPEHSPSESQPSSPSPTPSPSPV |
| | | LEPGSEPNLAVLSIPGDTMTTIQ |
| | FCHO1_0 | SPENVEDSGLDSPSHAAPGPSPDSWVPRPGTPQSPPS |
| | | CRAPPPEARGIRAPPLPDSPQPLAS |
| | FCHO1_1 | QSPPSCRAPPPEARGIRAPPLPDSPQPLASSPGPWGLE |
| | | ALAGGDLMPAPADPTAREGLAAPP |
| | USP25 | LSYGSGPKRFPLVDVLQYALEFASSKPVCTSPVDDI |
| | | DASSPPSGSIPSQTLPSTTEQQGALS |
| | RXRB | EQQTPEPEPGEAGRDGMGDSGRDSRSPDSSSPNPLP |
| | | QGVPPPSPPGPPLPPSTAPSLGGSGA |
| | SNW1 | MQKDPMEPPRFKINKKIPRGPPSPPAPVMHSPSRKM |
| | | TVKEQQEWKIPPCISNWKNAKGYTIP |
| | APC_0 | KKQNLKNNSKVFNDKLPNNEDRVRGSFAFDSPHHY |
| | | TPIEGTPYCFSRNDSLSSLDFDDDDVD |
| | APC_1 | SRGRTMIHIPGVRNSSSSTSPVSKKGPPLKTPASKSPS |
| | | EGQTATTSPRGAKPSVKSELSPVA |
| | APC_2 | MIHIPGVRNSSSSTSPVSKKGPPLKTPASKSPSEGQT |
| | | ATTSPRGAKPSVKSELSPVARQTSQ |
| | APC_3 | SSSTSPVSKKGPPLKTPASKSPSEGQTATTSPRGAKP |
| | | SVKSELSPVARQTSQIGGSSKAPSR |
| 2288 | ARHGEF16 | MVRGSPRVRDDAAFQPQVPAPPQPRPPGHEEPWPIV |
| | | LSTESPAALKLGTQQLIPKSLAVASK |
| 2289 | CCNB1 | AKPSATGKVIDKKLPKPLEKVPMLVPVPVSEPVPEP |
| | | EPEPEPEPVKEEKLSPEPILVDTASP |
| 2290 | RNF43 | KRFQWHGRKPGPETGVPQSRPPIPRTQPQPEPPSPDQ |
| | | QVTRSNSAAPSGRLSNPQCPRALPE |
| | RAPGEF6 | SQSQDDSIVGTRHCRHSLAIMPIPGTLSSSSPDLLQPT |
| | | TSMLDFSNPSDIPDQVIRVFKVDQ |
| 2291 | SMTN_0 | PSSSPTPASPEPPLEPAEAQCLTAEVPGSPEPPPSPPKT |
| | | TSPEPQESPTLPSTEGQVVNKLL |
| | SMTN_1 | EPPLEPAEAQCLTAEVPGSPEPPPSPPKTTSPEPQESP |
| | | TLPSTEGQVVNKLLSGPKETPAAQ |
| | PKN1 | TGTLEVRVVGCRDLPETIPWNPTPSMGGPGTPDSRP |
| | | PFLSRPARGLYSRSGSLSGRSSLKAE |
| | ASXL2_0 | FQVSPQPFLNRGDRIQVRKVPPLKIPVSRISPMPFHPS |
| | | QVSPRARFPVSITSPNRTGARTLA |
| | ASXL2_1 | RGDRIQVRKVPPLKIPVSRISPMPFHPSQVSPRARFPV |
| | | SITSPNRTGARTLADIKAKAQLVK |
| | ASXL2_2 | FSSTVLPLPADSPTHQPLLLPPLQTPKLYGSPTQIGPS |
| | | YRGMINVSTSSDMDHNSAVPGSQV |
| | AOC1 | NENIENEDLVAWVTVGFLHIPHSEDIPNTATPGNSV |
| | | GFLLRPFNFFPEDPSLASRDTVIVWP |
| 2292 | TBX4 | MLQDKGLSESEEAFRAPGPALGEASAANAPEPALA |
| | | APGLSGAALGSPPGPGADVVAAAAAEQ |
| | MAP3K7 | ISGNGQPRRRSIQDLTVTGTEPGQVSSRSSSPSVRMIT |
| | | TSGPTSEKPTRSHPWTPDDSTDTN |
| | TEPSIN_0 | PLPGSQVFLQPLSSTPVSSRSPAPSSGMPSSPVPTPPP |
| | | DASPIPAPGDPSEAEARLAESRRW |
| 2293 | TEPSIN_1 | SSRSPAPSSGMPSSPVPTPPPDASPIPAPGDPSEAEAR |
| | | LAESRRWRPERIPGGTDSPKRGPS |
| | KIDINS220 | HSGKRGIPHSLSGLQDPIIARMSICSEDKKSPSECSLI |
| | | ASSPEENWPACQKAYNLNRTPSTV |
| | CAPRIN1_0 | FTSGEKEQVDEWTVETVEVVNSLQQQPQAASPSVP |
| | | EPHSLTPVAQADPLVRRQRVQDLMAQM |
| 2294 | CAPRIN1_1 | KEQVDEWTVETVEVVNSLQQQPQAASPSVPEPHSL |
| | | TPVAQADPLVRRQRVQDLMAQMQGPYN |
| | CAPRIN1_2 | EWTVETVEVVNSLQQQPQAASPSVPEPHSLTPVAQ |
| | | ADPLVRRQRVQDLMAQMQGPYNFIQDS |
| | TEAD4 | PGQAGTSHDVKPFSQQTYAVQPPLPLPGFESPAGPA |
| | | PSPSAPPAPPWQGRSVASSKLWMLEF |
| 2295 | ZNF687 | GRGTTLARGSSARAQGPGRKRRQSSDSCSEEPDSTT |
| | | PPAKSPRGGPGSGGHGPLRYRSSSST |
| | PRRC1 | PVRPSAPLPFVPPPAVPSVPPLVTSMPPPVSPSTAAAF |
| | | GNPPVSHFPPSTSAPNTLLPAPPS |
| | TMPRSS13_0 | SHGNASPARTPSAGASPAQASPAGTPPGRASPAQAS |
| | | PAQASPAGTPPGRASPAQASPAGTPP |
| | TMPRSS13_1 | SPARTPSAGASPAQASPAGTPPGRASPAQASPAQAS |
| | | PAGTPPGRASPAQASPAGTPPGRASP |
| | TMPRSS13_2 | PSAGASPAQASPAGTPPGRASPAQASPAQASPAGTP |
| | | PGRASPAQASPAGTPPGRASPGRASP |
| | TMPRSS13_3 | SPAGTPPGRASPAQASPAQASPAGTPPGRASPAQAS |
| | | PAGTPPGRASPGRASPAQASPAQASP |
| | TMPRSS13_4 | PPGRASPAQASPAQASPAGTPPGRASPAQASPAGTP |
| | | PGRASPGRASPAQASPAQASPARASP |
| | TMPRSS13_5 | SPAQASPAGTPPGRASPAQASPAGTPPGRASPGRASP |
| | | AQASPAQASPARASPALASLSRSSS |
| | TMPRSS13_6 | SPAGTPPGRASPAQASPAGTPPGRASPGRASPAQASP |
| | | AQASPARASPALASLSRSSSGRSSS |
| | TMPRSS13_7 | PPGRASPAQASPAGTPPGRASPGRASPAQASPAQAS |
| | | PARASPALASLSRSSSGRSSSARSAS |
| | TMPRSS13_8 | SPAQASPAGTPPGRASPGRASPAQASPAQASPARAS |
| | | PALASLSRSSSGRSSSARSASVTTSP |
| | TMPRSS13_9 | SPAGTPPGRASPGRASPAQASPAQASPARASPALAS |
| | | LSRSSSGRSSSARSASVTTSPTRVYL |
| | TMPRSS13_10 | SLSRSSSGRSSSARSASVTTSPTRVYLVRATPVGAVP |
| | | IRSSPARSAPATRATRESPGTSLPK |
| | TMPRSS13_11 | SSARSASVTTSPTRVYLVRATPVGAVPIRSSPARSAP |
| | | ATRATRESPGTSLPKFTWREGQKQL |
| | SUPT5H_0 | THSPASYHPTPSPMAYQASPSPSPVGYSPMTPGAPSP |
| | | GGYNPHTPGSGIEQNSSDWVTTDIQ |
| | SUPT5H_1 | SYHPTPSPMAYQASPSPSPVGYSPMTPGAPSPGGYN |
| | | PHTPGSGIEQNSSDWVTTDIQVKVRD |
| 2296 | ZNF750 | HGLATIYSPYLLAGSSPECDAPLLSVYGTQDPRHFLP |
| | | HPGPIPKHLAPSPATYDHYRFFQQY |
| 2297 | TJP1_0 | TPVKHADDHTPKTVEEVTVERNEKQTPSLPEPKPVY |
| | | AQVGQPDVDLPVSPSDGVLPNSTHED |
| 2298 | TJP1_1 | PPFDNQHSQDLDSRQHPEESSERGYFPRFEEPAPLSY |
| | | DSRPRYEQAPRASALRHEEQPAPGY |
| | SOX5 | ATAGVVYPGAIAMAGMPSPHLPSEHSSVSSSPEPGM |
| | | PVIQSTYGVKGEEPHIKEEIQAEDIN |
| 2299 | CSF1 | CNNSFAECSSQDVVTKPDCNCLYPKAIPSSDPASVSP |
| | | HQPLAPSMAPVAGLTWEDSEGTEGS |
| 2300 | AIRE_0 | SPPLREIPSGTWRCSSCLQATVQEVQPRAEEPRPQEP |
| | | PVETPLPPGLRSAGEEVRGPPGEPL |
| 2301 | AIRE_1 | EIPSGTWRCSSCLQATVQEVQPRAEEPRPQEPPVETP |
| | | LPPGLRSAGEEVRGPPGEPLAGMDT |
| | AIRE_2 | TWRCSSCLQATVQEVQPRAEEPRPQEPPVETPLPPG |
| | | LRSAGEEVRGPPGEPLAGMDTTLVYK |
| | SEC16A_0 | HGGHPHGNMPGLDRPLSRQNPHDGVVTPAASPSLP |
| | | QPGLQMPGQWGPVQGGPQPSGQHRSPC |
| | SEC16A_1 | PDGPLASPARVPMFPVPLPPGPLEPGPGCVTPGPALG |
| | | FLEPSGPGLPPGVPPLQERRHLLQE |
| | SEC16A_2 | GTQRSEPALAPADFVAPLAPLPIPSNLFVPTPDAEEP |
| | | QLPDGTGREGPAAARGLANPEPAPE |
| 2302 | SEC16A_3 | PDAEEPQLPDGTGREGPAAARGLANPEPAPEPKVLS |
| | | SAASLPGSELPSSRPEGSQGGELSRC |
| 2303 | MYO18B_0 | LGSSATPTKKTVPFKRGVRRGDVLLMVAKLDPDSA |
| | | KPEKTHPHDAPPCKTSPPATDTGKEKK |
| | MYO18B_1 | GDVLLMVAKLDPDSAKPEKTHPHDAPPCKTSPPAT |
| | | DTGKEKKGETSRTPCGSQASTEILAPK |
| 2304 | MYO18B_2 | GTVALKKGEEGQSIVGKGLGTPKTTELKEAEPQGK |
| | | DRQGTRPQAQGPGEGVRPGKAEKEGAE |
| | NAV2 | NSVKVNPAAQPVSSPAQTSLQPGAKYPDVASPTLRR |
| | | LFGGKPTKQVPIATAENMKNSVVISN |
| 2305 | DYSF | KQPTGASLVLQVSYTPLPGAVPLFPPPTPLEPSPTLP |
| | | DLDVVADTGGEEDTEDQGLTGDEAE |
| 2306 | USP28_0 | VMRNHWCSYLGQDIAENLQLCLGEFLPRLLDPSAEI |
| | | IVLKEPPTIRPNSPYDLCSRFAAVME |
| 2307 | USP28_1 | GQDIAENLQLCLGEFLPRLLDPSAEIIVLKEPPTIRPN |
| | | SPYDLCSRFAAVMESIQGVSTVTV |
| | TCF7L2_0 | LEEAAKRQDGGLFKGPPYPGYPFIMIPDLTSPYLPNG |
| | | SLSPTARTLHFQSGSTHYSAYKTIE |
| 2308 | TCF7L2_1 | HHVHPLTPLITYSNEHFTPGNPPPHLPADVDPKTGIP |
| | | RPPHPPDISPYYPLSPGTVGQIPHP |
| | TCF7L2_2 | HFTPGNPPPHLPADVDPKTGIPRPPHPPDISPYYPLSP |
| | | GTVGQIPHPLGWLVPQQGQPVYPI |
| 2309 | CHEK2_0 | SSQSSHSSSGTLSSLETVSTQELYSIPEDQEPEDQEPE |
| | | EPTPAPWARLWALQDGFANLECVN |
| 2310 | CHEK2_1 | HSSSGTLSSLETVSTQELYSIPEDQEPEDQEPEEPTPA |
| | | PWARLWALQDGFANLECVNDNYWF |
| | CHEK2_2 | TLSSLETVSTQELYSIPEDQEPEDQEPEEPTPAPWAR |
| | | LWALQDGFANLECVNDNYWFGRDKS |
| | IL15RA_0 | CIRDPALVHQRPAPPSTVTTAGVTPQPESLSPSGKEP |
| | | AASSPSSNNTAATTAAIVPGSQLMP |
| 2311 | IL15RA_1 | ALVHQRPAPPSTVTTAGVTPQPESLSPSGKEPAASSP |
| | | SSNNTAATTAAIVPGSQLMPSKSPS |
| | IL15RA_2 | RPAPPSTVTTAGVTPQPESLSPSGKEPAASSPSSNNT |
| | | AATTAAIVPGSQLMPSKSPSTGTTE |
| 2312 | ZSWIM8 | YSVTPPSLAATAVSFPVPSMAPITVHPYHTEPGLPLP |
| | | TSVACELWGQGTVSSVHPASTFPAI |
| | UHRF2 | LNDIIQLLVRPDPDHLPGTSTQIEAKPCSNSPPKVKK |
| | | APRVGPSNQPSTSARARLIDPGFGI |
| | PDLIM2_0 | DSSLEVLATRFQGSVRTYTESQSSLRSSYSSPTSLSPR |
| | | AGSPFSPPPSSSSLTGEAAISRSF |
| | PDLIM2_1 | VLATRFQGSVRTYTESQSSLRSSYSSPTSLSPRAGSPF |
| | | SPPPSSSSLTGEAAISRSFQSLAC |
| | PDLIM2_2 | FQGSVRTYTESQSSLRSSYSSPTSLSPRAGSPFSPPPSS |
| | | SSLTGEAAISRSFQSLACSPGLP |
| | PNPLA6 | HNYLGLTNELFSHEIQPLRLFPSPGLPTRTSPVRGSK |
| | | RMVSTSATDEPRETPGRPPDPTGAP |
| | GP1BA_0 | TQESTKEQTTFPPRWTPNFTLHMESITFSKTPKSTTE |
| | | PTPSPTTSEPVPEPAPNMTTLEPTP |
| 2313 | GP1BA_1 | TPNFTLHMESITFSKTPKSTTEPTPSPTTSEPVPEPAP |
| | | NMTTLEPTPSPTTPEPTSEPAPSP |
| | GP1BA_2 | TPKSTTEPTPSPTTSEPVPEPAPNMTTLEPTPSPTTPEP |
| | | TSEPAPSPTTPEPTSEPAPSPTT |
| | GP1BA_3 | TEPTPSPTTSEPVPEPAPNMTTLEPTPSPTTPEPTSEPA |
| | | PSPTTPEPTSEPAPSPTTPEPTS |
| | GP1BA_4 | EPVPEPAPNMTTLEPTPSPTTPEPTSEPAPSPTTPEPTS |
| | | EPAPSPTTPEPTSEPAPSPTTPE |
| | GP1BA_5 | PEPAPNMTTLEPTPSPTTPEPTSEPAPSPTTPEPTSEPA |
| | | PSPTTPEPTSEPAPSPTTPEPTP |
| | GP1BA_6 | EPTPSPTTPEPTSEPAPSPTTPEPTSEPAPSPTTPEPTSE |
| | | PAPSPTTPEPTPIPTIATSPTI |
| | GP1BA_7 | PSPTTPEPTSEPAPSPTTPEPTSEPAPSPTTPEPTSEPAP |
| | | SPTTPEPTPIPTIATSPTILVS |
| 2314 | GP1BA_8 | PTTPEPTSEPAPSPTTPEPTSEPAPSPTTPEPTSEPAPSP |
| | | TTPEPTPIPTIATSPTILVSAT |
| | GPIBA_9 | EPAPSPTTPEPTSEPAPSPTTPEPTSEPAPSPTTPEPTPI |
| | | PTIATSPTILVSATSLITPKST |
| 2315 | GP1BA_10 | PTTPEPTSEPAPSPTTPEPTSEPAPSPTTPEPTPIPTIAT |
| | | SPTILVSATSLITPKSTFLTTT |
| 2316 | PNPO | KDGFRFFTNFESRKGKELDSNPFASLVFYWEPLNRQ |
| | | VRVEGPVKKLPEEEAECYFHSRPKSS |
| | ADAMTS7 | FLPEEDTPIGAPDLGLPSLSWPRVSTDGLQTPATPES |
| | | QNDFPVGKDSQSQLPPPWRDRTNEV |
| | TRIB1 | LDADDAAAVAAKCPRLSECSSPPDYLSPPGSPCSPQ |
| | | PPPAAPGAGGGSGSAPGPSRIADYLL |
| 2317 | RRS1 | LARDNTQLLINQLWQLPTERVEEAIVARLPEPTTRLP |
| | | REKPLPRPRPLTRWQQFARLKGIRP |
| | GMEB1 | QNVVLMPVSTPKPPKRPRLQRPASTTVLSPSPPVQQ |
| | | PQFTVISPITITPVGQSFSMGNIPVA |
| | RNF213 | LPRGLQVGQPNLVVCGHSEVLPAALAVYMQTPSQP |
| | | LPTYDEVLLCTPATTFEEVALLLRRCL |
| | IFI16_0 | ALSRKRKKEVDATSPAPSTSSTVKTEGAEATPGAQN |
| | | PKTVAKCQVTPRRNVLQKRPVIVKVL |
| | IFI16_1 | LKEGSHFPGPFMTSIGPAESHPHTPQMPPSTPSSSFLT |
| | | TLKPRLKTEPEEVSIEDSAQSDLK |
| 2318 | EOGT | LMLFVFGVLLHEVSLSGQNEAPPNTHSIPGEPLYNY |
| | | ASIRLPEEHIPFFLHNNRHIATVCRK |
| | KDM2A_0 | KAQKRKMEESDEEAVQAKVLRPLRSCDEPLTPPPHS |
| | | PTSMLQLIHDPVSPRGMVTRSSPGAG |
| | KDM2A_1 | KMEESDEEAVQAKVLRPLRSCDEPLTPPPHSPTSML |
| | | QLIHDPVSPRGMVTRSSPGAGPSDHH |
| 2319 | AHCYL2 | LKDLSPSEAESQLGLSTAAVGAMAPPAGGGDPEAP |
| | | APAAERPPVPGPGSGPAAALSPAAGKV |
| | NRK | ASAILYAGFVEVPEESPKQPSEVNVNPLYVSPACKK |
| | | PLIHMYEKEFTSEICCGSLWGVNLLL |
| | CGNL1 | SNWLKTLTEEGINNKKPWTCFPKPSNSQPTSPSLEDP |
| | | AKSGVTAIRLCSSVVIEDPKKQTSV |
| | DMTN | STSPPPSPEVWADSRSPGIISQASAPRTTGTPRTSLPH |
| | | FHHPETSRPDSNIYKKPPIYKQRE |
| 2320 | B4GALNT4 | EDEVQRRAFLFLNPDDFLDDEDEGELLDSLEPTEAA |
| | | PPRSGPQSPAPAAPAQPGATLAPPTP |
| | PABPC4 | TAVQNLAPRAAVAAAAPRAVAPYKYASSVRSPHPA |
| | | IQPLQAPQPAVHVQGQEPLTASMLAAA |
| 2321 | E2F1_0 | SSQIVIISAAQDASAPPAPTGPAAPAAGPCDPDLLLF |
| | | ATPQAPRPTPSAPRPALGRPPVKRR |
| | E2F1_1 | AAQDASAPPAPTGPAAPAAGPCDPDLLLFATPQAPR |
| | | PTPSAPRPALGRPPVKRRLDLETDHQ |
| | E2F1_2 | PPAPTGPAAPAAGPCDPDLLLFATPQAPRPTPSAPRP |
| | | ALGRPPVKRRLDLETDHQYLAESSG |
| 2322 | KPRP_0 | QHRSRSTSRCLPPPRRLQLFPRSCSPPRRFEPCSSSYL |
| | | PLRPSEGFPNYCTPPRRSEPIYNS |
| | KPRP_1 | GASCPELRPHVEPRPLPSFCPPRRLDQCPESPLQRCPP |
| | | PAPRPRLRPEPCISLEPRPRPLPR |
| | AGER | EEVQLVVEPEGGAVAPGGTVTLTCEVPAQPSPQIHW |
| | | MKDGVPLPLPPSPVLILPEIGPQDQG |
| 2323 | KHSRP_0 | PPHAGGPPPHQYPPQGWGNTYPQWQPPAPHDPSKA |
| | | AAAAADPNAAWAAYYSHYYQQPPGPVP |
| 2324 | KHSRP_1 | QYPPQGWGNTYPQWQPPAPHDPSKAAAAAADPNA |
| | | AWAAYYSHYYQQPPGPVPGPAPAPAAPP |
| 2325 | KHSRP_2 | WAAYYSHYYQQPPGPVPGPAPAPAAPPAQGEPPQP |
| | | PPTGQSDYTKAWEEYYKKIGQQPQQPG |
| 2326 | ABLIM1 | FTAHRRATITHLLYLCPKDYCPRGRVCNSVDPFVAH |
| | | PQDPHHPSEKPVIHCHKCGEPCKGEV |
| | SIK3 | AAGAGTGGAGPAGRLLPPPAPGSPAAPAAVSPAAG |
| | | QPRPPAPASRGPMPARIGYYEIDRTIG |
| | TAF4B | GETSGAAICLPSVKPVVSSAGTTSDKPVIGTPVQIKL |
| | | AQPGPVLSQPAGIPQAVQVKQLVVQ |
| | AKNA | PIMPYPPAAVYYAPAGPTSAQPAAKWPPTASPPPAR |
| | | RHRHSIQLDLGDLEELNKALSRAVQA |
| | NUP62 | STAQPSGFNIGSAGNSAQPTAPATLPFTPATPAATTA |
| | | GATQPAAPTPTATITSTGPSLFASI |
| 2327 | LATS2 | HVAFRPDCPVPSRTNSFNSHQPRPGPPGKAEPSLPAP |
| | | NTVTAVTAAHILHPVKSVRVLRPEP |
| | ARHGAP33_0 | RAGGGGRDAPEAAAQSPCSVPSQVPTPGFFSPAPRE |
| | | CLPPFLGVPKPGLYPLGPPSFQPSSP |
| | ARHGAP33_1 | TRSWSPFRSMPPDRLNASYGMLGQSPPLHRSPDFLL |
| | | SYPPAPSCFPPDHLGYSAPQHPARRP |
| | ARHGAP33_2 | PARRPTPPEPLYVNLALGPRGPSPASSSSSSPPAHPRS |
| | | RSDPGPPVPRLPQKQRAPWGPRTP |
| 2328 | TEAD2_0 | PPWNVPDVKPFSQTPFTLSLTPPSTDLPGYEPPQALS |
| | | PLPPPTPSPPAWQARGLGTARLQLV |
| | TEAD2_1 | DVKPFSQTPFTLSLTPPSTDLPGYEPPQALSPLPPPTP |
| | | SPPAWQARGLGTARLQLVEFSAFV |
| | TP53BP1_0 | EEGGEPFQKKLQSGEPVELENPPLLPESTVSPQASTPI |
| | | SQSTPVFPPGSLPIPSQPQFSHDI |
| | TP53BP1_1 | PFQKKLQSGEPVELENPPLLPESTVSPQASTPISQSTP |
| | | VFPPGSLPIPSQPQFSHDIFIPSP |
| | PPP1R13B_0 | LERRKEGSLPRPSAGLPSRQRPTLLPATGSTPQPGSS |
| | | QQIQQRISVPPSPTYPPAGPPAFPA |
| | PPP1R13B_1 | PSESTEKEPEQDGPAAPADGSTVESLPRPLSPTKLTPI |
| | | VHSPLRYQSDADLEALRRKLANAP |
| | PPP1R13B_2 | EKEPEQDGPAAPADGSTVESLPRPLSPTKLTPIVHSP |
| | | LRYQSDADLEALRRKLANAPRPLKK |
| | PPP1R13B_3 | QDGPAAPADGSTVESLPRPLSPTKLTPIVHSPLRYQS |
| | | DADLEALRRKLANAPRPLKKRSSIT |
| | EML3_0 | QEMELVKAALAEALRLLRLQVPPSSLQGSGTPAPPG |
| | | DSLAAPPGLPPTCTPSLVSRGTQTET |
| | EML3_1 | SEGGGSSSSGAGSPGPPGILRPLQPPQRADTPRRNSS |
| | | SSSSPSERPRQKLSRKAISSANLLV |
| | ZDHHC8_0 | SLSYDSLLNPGSPGGHACPAHPAVGVAGYHSPYLHP |
| | | GATGDPPRPLPRSFSPVLGPRPREPS |
| 2329 | ZDHHC8_1 | GSPGGHACPAHPAVGVAGYHSPYLHPGATGDPPRP |
| | | LPRSFSPVLGPRPREPSPVRYDNLSRT |
| | HIF3A_0 | QLNASEQLPRAYHRPLGAVPRPRARSFHGLSPPALE |
| | | PSLLPRWGSDPRLSCSSPSRGDPSAS |
| 2330 | HIF3A_1 | EQLPRAYHRPLGAVPRPRARSFHGLSPPALEPSLLPR |
| | | WGSDPRLSCSSPSRGDPSASSPMAG |
| 2331 | HIF3A_2 | LFPLSLSFLLTGGPAPGSLQDPSTPLLNLNEPLGLGPS |
| | | LLSPYSDEDTTQPGGPFQPRAGSA |
| 2332 | HUS1 | ELLSMSSSSRIVTHDIPIKVIPRKLWKDLQEPVVPDP |
| | | DVSIYLPVLKTMKSVVEKMKNISNH |
| 2333 | ZNF385A_0 | NSQSQAEAHYKGNRHARRVKGIEAAKTRGREPGVR |
| | | EPGDPAPPGSTPTNGDGVAPRPVSMEN |
| 2334 | ZNF385A_1 | AEAHYKGNRHARRVKGIEAAKTRGREPGVREPGDP |
| | | APPGSTPTNGDGVAPRPVSMENGLGPA |
| | ZNF385A_2 | ARRVKGIEAAKTRGREPGVREPGDPAPPGSTPTNGD |
| | | GVAPRPVSMENGLGPAPGSPEKQPGS |
| 2335 | ZNF385A_3 | KGTKHKTILEARSGLGPIKAYPRLGPPTPGEPEAPAQ |
| | | DRTFHCEICNVKVNSEVQLKQHISS |
| | ZNF385A_4 | TFSKELPKSLAGGLLPSPLAVAAVMAAAAGSPLSLR |
| | | PAPAAPLLQGPPITHPLLHPAPGPIR |
| | VASN_0 | ATTTTATVPTTRPVVREPTALSSSLAPTWLSPTEPAT |
| | | EAPSPPSTAPPTVGPVPQPQDCPPS |
| | VASN_1 | TRPVVREPTALSSSLAPTWLSPTEPATEAPSPPSTAPP |
| | | TVGPVPQPQDCPPSTCLNGGTCHL |
| | MYRF_0 | CFPDISAPASSASYSHGQPAMPGSSGVHHLSPPGGGP |
| | | SPGRHGPLPPPGYGTPLNCNNNNGM |
| 2336 | MYRF_1 | YGTPLNCNNNNGMGAAPKPFPGGTGPPIKAEPKAP |
| | | YAPGTLPDSPPDSGSEAYSPQQVNEPH |
| | MYRF_2 | PTRAPSPPWPPQGPLSPGPGSLPLSIARVQTPPWHPP |
| | | GAPSPGLLQDSDSLSGSYLDPNYQS |
| | MAP2K7 | RRRIDLNLDISPQRPRPTLQLPLANDGGSRSPSSESSP |
| | | QHPTPPARPRHMLGLPSTLFTPRS |
| 2337 | BOP1 | PAYGRFIQERFERCLDLYLCPRQRKMRVNVDPEDLI |
| | | PKLPRPRDLQPFPTCQALVYRGHSDL |
| | RORC | VVKTPPAGAQGADTLTYTLGLPDGQLPLGSSPDLPE |
| | | ASACPPGLLKASGSGPSYSNNLAKAG |
| 2338 | TRERF1_0 | NPNPAASYSGATLYQSQLRSPRVLGDHLLLDPTHEL |
| | | PPYTPPPMLSPVRQGSGLFSNVLISG |
| | TRERF1_1 | SQLRSPRVLGDHLLLDPTHELPPYTPPPMLSPVRQGS |
| | | GLFSNVLISGHGPGAHPQLPLTPLT |
| | EIF4B | TSTTSSRNARRRESEKSLENETLNKEEDCHSPTSKPP |
| | | KPDQPLKVMPAPPPKENAWVKRSSN |
| | MAP7D1_0 | RAGASLARGPQPDRTHPSAAVPVCPRSASASPLTPC |
| | | SVTRSVHRCAPAGERGERRKPNAGGS |
| | MAP7D1_1 | GPEDKSQSKRRASNEKESAAPASPAPSPAPSPTPAPP |
| | | QKEQPPAETPTDAAVLTSPPAPAPP |
| | MAP7D1_2 | KESAAPASPAPSPAPSPTPAPPQKEQPPAETPTDAAV |
| | | LTSPPAPAPPVTPSKPMAGTTDREE |
| 2339 | MAP7D1_3 | EANANGSSPEPVKAVEARSPGLQKEAVQKEEPIPQE |
| | | PQWSLPSKELPASLVNGLQPLPAHQE |
| 2340 | MAP7D1_4 | GSSPEPVKAVEARSPGLQKEAVQKEEPIPQEPQWSL |
| | | PSKELPASLVNGLQPLPAHQENGFST |
| | RAB11FIP5_0 | ASPHHSSSGEEKAKSSWFGLREAKDPTQKPSPHPVK |
| | | PLSAAPVEGSPDRKQSRSSLSIALSS |
| | RAB11FIP5_1 | SWFGLREAKDPTQKPSPHPVKPLSAAPVEGSPDRKQ |
| | | SRSSLSIALSSGLEKLKTVTSGSIQP |
| | RAD54L2 | LSEPRMFAPFPSPVLPSNLSRGMSIYPGYMSPHAGYP |
| | | AGGLLRSQVPPFDSHEVAEVGFSSN |
| | LZTS2 | CPSGTLSDSGRNSLSSLPTYSTGGAEPTTSSPGGHLP |
| | | SHGSGRGALPGPARGVPTGPSHSDS |
| | SH3BP1_0 | SGSPGTPQALPRRLVGSSLRAPTVPPPLPPTPPQPAR |
| | | RQSRRSPASPSPASPGPASPSPVSL |
| | SH3BP1_1 | RLVGSSLRAPTVPPPLPPTPPQPARRQSRRSPASPSPA |
| | | SPGPASPSPVSLSNPAQVDLGAAT |
| | SH3BP1_2 | GSSLRAPTVPPPLPPTPPQPARRQSRRSPASPSPASPG |
| | | PASPSPVSLSNPAQVDLGAATAEG |
| | SH3BP1_3 | SLRAPTVPPPLPPTPPQPARRQSRRSPASPSPASPGPA |
| | | SPSPVSLSNPAQVDLGAATAEGGA |
| | SH3BP1_4 | LPPTPPQPARRQSRRSPASPSPASPGPASPSPVSLSNP |
| | | AQVDLGAATAEGGAPEAISGVPTP |
| 2341 | L3MBTL1_0 | FWIDADHPDIHPAGWCSKTGHPLQPPLGPREPSSASP |
| | | GGCPPLSYRSLPHTRTSKYSFHHRK |
| | L3MBTL1_1 | DHPDIHPAGWCSKTGHPLQPPLGPREPSSASPGGCPP |
| | | LSYRSLPHTRTSKYSFHHRKCPTPG |
| | NBEAL2_0 | ARQAGWQDVLTRLYVLEAATAGSPPPSSPESPTSPK |
| | | PAPPKPPTESPAEPSDVFLPSEAPCP |
| | NBEAL2_1 | AGWQDVLTRLYVLEAATAGSPPPSSPESPTSPKPAPP |
| | | KPPTESPAEPSDVFLPSEAPCPDPD |
| | NBEAL2_2 | LEAATAGSPPPSSPESPTSPKPAPPKPPTESPAEPSDV |
| | | FLPSEAPCPDPDGFYHALSPFCTP |
| 2342 | NBEAL2_3 | ATAGSPPPSSPESPTSPKPAPPKPPTESPAEPSDVFLPS |
| | | EAPCPDPDGFYHALSPFCTPFDL |
| | TP53 | EQWFTEDPGPDEAPRMPEAAPPVAPAPAAPTPAAPA |
| | | PAPSWPLSSSVPSQKTYQGSYGFRLG |
| | RGL3 | LSAKLAREKSSSPSGSPGDPSSPTSSVSPGSPPSSPRS |
| | | RDAPAGSPPASPGPQGPSTKLPLS |
| | PRG4_0 | TPKAETTTKGPALTTPKEPTPTTPKEPASTTPKEPTPT |
| | | TIKSAPTTPKEPAPTTTKSAPTTP |
| | PRG4_1 | TTTKGPALTTPKEPTPTTPKEPASTTPKEPTPTTIKSA |
| | | PTTPKEPAPTTTKSAPTTPKEPAP |
| | PRG4_2 | PKEPTPTTPKEPASTTPKEPTPTTIKSAPTTPKEPAPTT |
| | | TKSAPTTPKEPAPTTTKEPAPTT |
| | PRG4_3 | TPKEPTPTTIKSAPTTPKEPAPTTTKSAPTTPKEPAPT |
| | | TTKEPAPTTPKEPAPTTTKEPAPT |
| 2343 | PRG4_4 | PAPTTPKEPAPTTTKEPAPTTTKSAPTTPKEPAPTTPK |
| | | KPAPTTPKEPAPTTPKEPTPTTPK |
| 2344 | PRG4_5 | APTTPKEPAPTTPKKPAPTTPKEPAPTTPKEPTPTTPK |
| | | EPAPTTKEPAPTTPKEPAPTAPKK |
| | PRG4_6 | TTPKEPAPTTPKKPAPTTPKEPAPTTPKEPTPTTPKEP |
| | | APTTKEPAPTTPKEPAPTAPKKPA |
| | PRG4_7 | KEPAPTTPKKPAPTTPKEPAPTTPKEPTPTTPKEPAPT |
| | | TKEPAPTTPKEPAPTAPKKPAPTT |
| 2345 | PRG4_8 | PAPTTPKEPAPTTPKEPTPTTPKEPAPTTKEPAPTTPK |
| | | EPAPTAPKKPAPTTPKEPAPTTPK |
| | PRG4_9 | PKEPAPTTPKEPTPTTPKEPAPTTKEPAPTTPKEPAPT |
| | | APKKPAPTTPKEPAPTTPKEPAPT |
| 2346 | PRG4_10 | PAPTTPKEPTPTTPKEPAPTTKEPAPTTPKEPAPTAPK |
| | | KPAPTTPKEPAPTTPKEPAPTTTK |
| 2347 | PRG4_11 | APTTPKEPAPTTPKETAPTTPKGTAPTTLKEPAPTTP |
| | | KKPAPKELAPTTTKEPTSTTSDKPA |
| | PRG4_12 | KEPAPTTPKETAPTTPKGTAPTTLKEPAPTTPKKPAP |
| | | KELAPTTTKEPTSTTSDKPAPTTPK |
| 2348 | PRG4_13 | APTTPKEPAPTTPKEPAPTTPKGTAPTTLKEPAPTTP |
| | | KKPAPKELAPTTTKGPTSTTSDKPA |
| | PRG4_14 | KEPAPTTPKEPAPTTPKGTAPTTLKEPAPTTPKKPAP |
| | | KELAPTTTKGPTSTTSDKPAPTTPK |
| | NHS | AGLASPSSGYSSQSETPTSSFPTAFFSGPLSPGGSKRK |
| | | PKVPERKSSLQQPSLKDGTISLSK |
| | TNK2_0 | SAQTAEIFQALQQECMRQLQAPAGSPAPSPSPGGDD |
| | | KPQVPPRVPIPPRPTRPHVQLSPAPP |
| | TNK2_1 | PIPPRPTRPHVQLSPAPPGEEETSQWPGPASPPRVPPR |
| | | EPLSPQGSRTPSPLVPPGSSPLPP |
| | TNK2_2 | STHYYLLPERPSYLERYQRFLREAQSPEEPTPLPVPL |
| | | LLPPPSTPAPAAPTATVRPMPQAAL |
| | TNK2_3 | LERYQRFLREAQSPEEPTPLPVPLLLPPPSTPAPAAPT |
| | | ATVRPMPQAALDPKANFSTNNSNP |
| 2349 | KMT2D_0 | KGGHVTSMQPKEPGPLQCEAKPLGKAGVQLEPQLE |
| | | APLNEEMPLLPPPEESPLSPPPEESPT |
| | KMT2D_1 | KPLGKAGVQLEPQLEAPLNEEMPLLPPPEESPLSPPP |
| | | EESPTSPPPEASRLSPPPEELPASP |
| | KMT2D_2 | LEPQLEAPLNEEMPLLPPPEESPLSPPPEESPTSPPPEA |
| | | SRLSPPPEELPASPLPEALHLSR |
| | KMT2D_3 | PEASRLSPPPEELPASPLPEALHLSRPLEESPLSPPPEE |
| | | SPLSPPPESSPFSPLEESPLSPP |
| | KMT2D_4 | PESSPFSPLEESPLSPPEESPPSPALETPLSPPPEASPLS |
| | | PPFEESPLSPPPEELPTSPPPE |
| | KMT2D_5 | PPEESPPSPALETPLSPPPEASPLSPPFEESPLSPPPEEL |
| | | PTSPPPEASRLSPPPEESPMSP |
| | KMT2D_6 | FEESPLSPPPEELPTSPPPEASRLSPPPEESPMSPPPEES |
| | | PMSPPPEASRLFPPFEESPLSP |
| | KMT2D_7 | PEELPTSPPPEASRLSPPPEESPMSPPPEESPMSPPPEA |
| | | SRLFPPFEESPLSPPPEESPLSP |
| | KMT2D_8 | PEESPMSPPPEESPMSPPPEASRLFPPFEESPLSPPPEE |
| | | SPLSPPPEASRLSPPPEDSPMSP |
| | KMT2D_9 | PEESPMSPPPEASRLFPPFEESPLSPPPEESPLSPPPEAS |
| | | RLSPPPEDSPMSPPPEESPMSP |
| | KMT2D_10 | FEESPLSPPPEESPLSPPPEASRLSPPPEDSPMSPPPEES |
| | | PMSPPPEVSRLSPLPVVSRLSP |
| | KMT2D_11 | PEESPLSPPPEASRLSPPPEDSPMSPPPEESPMSPPPEV |
| | | SRLSPLPVVSRLSPPPEESPLSP |
| | KMT2D_12 | PEESPMSPPPEVSRLSPLPVVSRLSPPPEESPLSPPPEE |
| | | SPTSPPPEASRLSPPPEDSPTSP |
| | KMT2D_13 | PEVSRLSPLPVVSRLSPPPEESPLSPPPEESPTSPPPEA |
| | | SRLSPPPEDSPTSPPPEDSPASP |
| | KMT2D_14 | PEESPLSPPPEESPTSPPPEASRLSPPPEDSPTSPPPEDS |
| | | PASPPPEDSLMSLPLEESPLLP |
| | KMT2D_15 | PEESPTSPPPEASRLSPPPEDSPTSPPPEDSPASPPPEDS |
| | | LMSLPLEESPLLPLPEEPQLCP |
| | KMT2D_16 | PEDSPTSPPPEDSPASPPPEDSLMSLPLEESPLLPLPEE |
| | | PQLCPRSEGPHLSPRPEEPHLSP |
| | KMT2D_17 | GEPALSEPGEPPLSPLPEELPLSPSGEPSLSPQLMPPD |
| | | PLPPPLSPIITAAAPPALSPLGEL |
| 2350 | KMT2D_18 | GAKGDSDPESPLAAPILETPISPPPEANCTDPEPVPPM |
| | | ILPPSPGSPVGPASPILMEPLPPQ |
| | KMT2D_19 | ILETPISPPPEANCTDPEPVPPMILPPSPGSPVGPASPIL |
| | | MEPLPPQCSPLLQHSLVPQNSP |
| | KMT2D_20 | SPILMEPLPPQCSPLLQHSLVPQNSPPSQCSPPALPLS |
| | | VPSPLSPIGKVVGVSDEAELHEME |
| | KMT2D_21 | DTAPLDGIDAPGSQPEPGQTPGSLASELKGSPVLLDP |
| | | EELAPVTPMEVYPECKQTAGQGSPC |
| 2351 | KMT2D_22 | PGELFLKLPPQVPAQVPSQDPFGLAPAYPLEPRFPTA |
| | | PPTYPPYPSPTGAPAQPPMLGASSR |
| | KMT2D_23 | CALPPRSLPSDPFSRVPASPQSQSSSQSPLTPRPLSAE |
| | | AFCPSPVTPRFQSPDPYSRPPSRP |
| | KMT2D_24 | FSRVPASPQSQSSSQSPLTPRPLSAEAFCPSPVTPRFQ |
| | | SPDPYSRPPSRPQSRDPFAPLHKP |
| | KMT2D_25 | VPASPQSQSSSQSPLTPRPLSAEAFCPSPVTPRFQSPD |
| | | PYSRPPSRPQSRDPFAPLHKPPRP |
| | KMT2D_26 | QSQSSSQSPLTPRPLSAEAFCPSPVTPRFQSPDPYSRP |
| | | PSRPQSRDPFAPLHKPPRPQPPEV |
| | KMT2D_27 | SAEAFCPSPVTPRFQSPDPYSRPPSRPQSRDPFAPLHK |
| | | PPRPQPPEVAFKAGSLAHTSLGAG |
| | KMT2D_28 | GAGPRPQGPPRLPAPPGALSTGPVLGPVHPTPPPSSP |
| | | QEPKRPSQLPSPSSQLPTEAQLPPT |
| | KMT2D_29 | PQGPPRLPAPPGALSTGPVLGPVHPTPPPSSPQEPKRP |
| | | SQLPSPSSQLPTEAQLPPTHPGTP |
| | KMT2D_30 | ALSTGPVLGPVHPTPPPSSPQEPKRPSQLPSPSSQLPT |
| | | EAQLPPTHPGTPKPQGPTLEPPPG |
| | KMT2D_31 | YTYNVSNLDVRQLSAPPPEEPSPPPSPLAPSPASPPTE |
| | | PLVELPTEPLAEPPVPSPLPLASS |
| 2352 | KMT2D_32 | LDVRQLSAPPPEEPSPPPSPLAPSPASPPTEPLVELPTE |
| | | PLAEPPVPSPLPLASSPESARPK |
| | ARHGAP32 | RFYSGDQPPSYLGASVDKLHHPLEFADKSPTPPNLPS |
| | | DKIYPPSGSPEENTSTATMTYMTTT |
| | ZNF652_0 | EKPYPCDVCGQRFRFSNMLKAHKEKCFRVTSPVNV |
| | | PPAVQIPLTTSPATPVPSVVNTATTPT |
| | ZNF652_1 | SNMLKAHKEKCFRVTSPVNVPPAVQIPLTTSPATPV |
| | | PSVVNTATTPTPPINMNPVSTLPPRP |
| | TNS2_0 | SYGGAVPSYCPAYGRVPHSCGSPGEGRGYPSPGAHS |
| | | PRAGSISPGSPPYPQSRKLSYEIPTE |
| | TNS2_1 | ASSELSGPSTPLHTSSPVQGKESTRRQDTRSPTSAPT |
| | | QRLSPGEALPPVSQAGTGKAPELPS |
| 2353 | TNS2_2 | TQRLSPGEALPPVSQAGTGKAPELPSGSGPEPLAPSP |
| | | VSPTFPPSSPSDWPQERSPGGHSDG |
| | TNS2_3 | PGEALPPVSQAGTGKAPELPSGSGPEPLAPSPVSPTF |
| | | PPSSPSDWPQERSPGGHSDGASPRS |
| | TNS2_4 | ALPPVSQAGTGKAPELPSGSGPEPLAPSPVSPTFPPSS |
| | | PSDWPQERSPGGHSDGASPRSPVP |
| | TNS2_5 | SPRSPVPTTLPGLRHAPWQGPRGPPDSPDGSPLTPVP |
| | | SQMPWLVASPEPPQSSPTPAFPLAA |
| 2354 | TNS2_6 | EPYFGSLSALVSQHSISPISLPCCLRIPSKDPLEETPEA |
| | | PVPTNMSTAADLLRQGAACSVLY |
| | TNS2_7 | SLSALVSQHSISPISLPCCLRIPSKDPLEETPEAPVPTN |
| | | MSTAADLLRQGAACSVLYLTSVE |
| 2355 | COL11A2_0 | QRERPQNQQPHRAQRSPQQQPSRLHRPQNQEPQSQP |
| | | TESLYYDYEPPYYDVMTTGTTPDYQD |
| 2356 | COL11A2_1 | ETELGPALSAETAHSGAAAHGPRGLKGEKGEPAVL |
| | | EPGMLVEGPPGPEGPAGLIGPPGIQGN |
| 2357 | COL11A2_2 | PALSAETAHSGAAAHGPRGLKGEKGEPAVLEPGML |
| | | VEGPPGPEGPAGLIGPPGIQGNPGPVG |
| | ARHGAP27_0 | LPSPVWETHTDAGTGRPYYYNPDTGVTTWESPFEA |
| | | AEGAASPATSPASVDSHVSLETEWGQY |
| 2358 | ARHGAP27_1 | TGETAWEDEAENEPEEELEMQPGLSPGSPGDPRPPT |
| | | PETDYPESLTSYPEEDYSPVGSFGEP |
| | ARHGAP27_2 | WEDEAENEPEEELEMQPGLSPGSPGDPRPPTPETDY |
| | | PESLTSYPEEDYSPVGSFGEPGPTSP |
| | FOXL1 | RSAEAQPEAGSGAGGSGPAISRLQAAPAGPSPLLDG |
| | | PSPPAPLHWPGTASPNEDAGDAAQGA |
| | TMEM132E | GPGGGEDEARGAGPPGSALPAPEAPGPGTASPVVPP |
| | | TEDFLPLPTGFLQVPRGLTDLEIGMY |
| 2359 | BAIAP2 | RAVQLMQQVASNGATLPSALSASKSNLVISDPIPGA |
| | | KPLPVPPELAPFVGRMSAQESTPIMN |
| | SOS1 | DYLFNKSLEIEPRNPKPLPRFPKKYSYPLKSPGVRPS |
| | | NPRPGTMRHPTPLQQEPRKISYSRI |
| | CRAMP1 | PSPRPGPGLLLDVCTKDLADAPAEELQEKGSPAGPP |
| | | PSQGQPAARPPKEVPASRLAQQLREE |
| | PIAS1 | EEPSAKRTCPSLSPTSPLNNKGILSLPHQASPVSRTPS |
| | | LPAVDTSYINTSLIQDYRHPFHMT |
| | PPP1R15B_0 | AGDIPGNTQESTEEKIELLTTEVPLALEEESPSEGCPS |
| | | SEIPMEKEPGEGRISVVDYSYLEG |
| 2360 | PPP1R15B_1 | IELLTTEVPLALEEESPSEGCPSSEIPMEKEPGEGRISV |
| | | VDYSYLEGDLPISARPACSNKLI |
| | JPH2_0 | LQEILENSESLLEPPDRGAGAAGLPQPPRESPQLHER |
| | | ETPRPEGGSPSPAGTPPQPKRPRPG |
| 2361 | JPH2_1 | DQPEPEVSGSESAPSSPATAPLQAPTLRGPEPARETP |
| | | AKLEPKPIIPKAEPRAKARKTEARG |
| | JPH2_2 | EVSGSESAPSSPATAPLQAPTLRGPEPARETPAKLEP |
| | | KPIIPKAEPRAKARKTEARGLTKAG |
| 2362 | JPH2_3 | ESAPSSPATAPLQAPTLRGPEPARETPAKLEPKPIIPK |
| | | AEPRAKARKTEARGLTKAGAKKKA |
| | PPFIBP2 | EEPEGGFSKWNATNKDPEELFKQEMPPRCSSPTVGP |
| | | PPLPQKSLETRAQKKLSCSLEDLRSE |
| | LPP_0 | IDSLTSILADLECSSPYKPRPPQSSTGSTASPPVSTPVT |
| | | GHKRMVIPNQPPLTATKKSTLKP |
| | LPP_1 | SILADLECSSPYKPRPPQSSTGSTASPPVSTPVTGHKR |
| | | MVIPNQPPLTATKKSTLKPQPAPQ |
| 2363 | NSD1 | KQHREGMLFISKLDGRLSCTEHDPCGPNPLEPGEIRE |
| | | YVPPPVPLPPGPSTHLAEQSTGMAA |
| | PMEL | QAVPSGEGDAFELTVSCQGGLPKEACMEISSPGCQP |
| | | PAQRLCQPVLPSPACQLVLHQILKGG |
| 2364 | LRFN1 | AAGEATAPVEVCVVPLPLMAPPPAAPPPLTEPGSSDI |
| | | ATPGRPGANDSAAERRLVAAELTSN |
| 2365 | RAD21 | GVMLPEQPAHDDMDEDDNVSMGGPDSPDSVDPVE |
| | | PMPTMTDQTTLVPNEEEAFALEPIDITV |
| 2366 | PGM2L1 | TSFHGVGHDYVQLAFKVFGFKPPIPVPEQKDPDPDF |
| | | STVKCPNPEEGESVLELSLRLAEKEN |
| | ITSN2 | SIAMKLIKLKLQGQQLPVVLPPIMKQPPMFSPLISAR |
| | | FGMGSMPNLSIPQPLPPAAPITSLS |
| | CSTF2 | EVRGMEARGMDTRGPVPGPRGPIPSGMQGPSPINM |
| | | GAVVPQGSRQVPVMQGTGMQGASIQGG |
| 2367 | BCL9L_0 | GAASTGGGTGGTHPNTPTATTANNPLPPGGDPSSAP |
| | | GPALLGEAAAPGNGQRSLVGSEGLSK |
| | BCL9L_1 | LTISINQMGSPGMGHLKSPTLSQVHSPLVTSPSANLK |
| | | SPQTPSQMVPLPSANPPGPLKSPQV |
| | BCL9L_2 | PGMGHLKSPTLSQVHSPLVTSPSANLKSPQTPSQMV |
| | | PLPSANPPGPLKSPQVLGSSLSVRSP |
| 2368 | BCL9L_3 | NSQPSQMHLNSAAAQSPMGMNLPGQQPLSHEPPPA |
| | | MLPSPTPLGSNIPLHPNAQGTGGPPQN |
| 2369 | ZNF142_0 | YVPGDQAWQLRYASQEPEGAMQGPTPPPDSEPSNQ |
| | | LSARPEGPGHEPGTVVDPSLDQALPEM |
| | ZNF142_1 | SFKQQRGLSTHLLKKCPVLLRKNKGLPRPDSPIPLQP |
| | | VLPGTQASEDTESGKPPPASQEAEL |
| | MED13L_0 | LNTPQMNTPVTLNSAAPASNSGAGVLPSPATPRFSV |
| | | PTPRTPRTPRTPRGGGTASGQGSVKY |
| | MED13L_1 | TLNSAAPASNSGAGVLPSPATPRFSVPTPRTPRTPRT |
| | | PRGGGTASGQGSVKYDSTDQGSPAS |
| | MED13L_2 | LYAQVCRHHLAPYLATLQLDSSLLIPPKYQTPPAAA |
| | | QGQATPGNAGPLAPNGSAAPPAGSAF |
| | MED13L_3 | APYLATLQLDSSLLIPPKYQTPPAAAQGQATPGNAG |
| | | PLAPNGSAAPPAGSAFNPTSNSSSTN |
| | MASTL | PNQIKSGTPYRTPKSVRRGVAPVDDGRILGTPDYLA |
| | | PELLLGRAHGPAVDWWALGVCLFEFL |
| 2370 | SAMD11_0 | YRRLVSALSEASTFEDPQRLYHLGLPSHGEDPPWHD |
| | | PPHHLPSHDLLRVRQEVAAAALRGPS |
| | SAMD11_1 | QGLAQHREGAAPAAAPSFSERELPQPPPLLSPQNAP |
| | | HVALGPHLRPPFLGVPSALCQTPGYG |
| 2371 | FLYWCH1 | RRQREKLPSLALPEGLGEPQGPEGPGGRVEEPLEGV |
| | | GPWQCPEEPEPTPGLVLSKPALEEEE |
| | BCORL1 | APVPTPVLAPMPASTPPAAPAPPSVPMPTPTPSSGPP |
| | | STPTLIPAFAPTPVPAPTPAPIFTP |
| | SETD1B_0 | RTKLLFLREPDSDTELQMEGSPISSSSSQLSPLAPFGT |
| | | NSQPGFRGPTPPSSRPSSTGLEDI |
| 2372 | SETD1B_1 | LSPEPPAKEVEARPPLSPERAPEHDLEVEPEPPMMLP |
| | | LPLQPPLPPPRPPRPPSPPPEPETT |
| | SETD1B_2 | HDLEVEPEPPMMLPLPLQPPLPPPRPPRPPSPPPEPET |
| | | TDASHPSVPPEPLAEDHPPHTPGL |
| | SETD1B_3 | TEEYMELAKSRGPWRRPPKKRHEDLVPPAGSPELSP |
| | | PQPLFRPRSEFEEMTILYDIWNGGID |
| | ZCCHC8 | GSQSSESFQFQPPLPPDTPPLPRGTPPPVFTPPLPKGT |
| | | PPLTPSDSPQTRTASGAVDEDALT |
| | IKBKG | RKRHVEVSQAPLPPAPAYLSSPLALPSQRRSPPEEPP |
| | | DFCCPKCQYQAPDMDTLQIHVMECI |
| | LASIL | ARRGWRLFNCSASLDWPRMVESCLGSPCWASPQLL |
| | | RIIFKAMGQGLPDEEQEKLLRICSIYT |
| | PDZD4_0 | PEKSDKDSTSAYNTGESCRSTPLLVEPLPESPLRRAM |
| | | AGNSNLNRTPPGPAVATPAKAAPPP |
| | PDZD4_1 | LVEPLPESPLRRAMAGNSNLNRTPPGPAVATPAKAA |
| | | PPPGSPAKFRSLSRDPEAGRRQHAEE |
| | PDZD4_2 | RRAMAGNSNLNRTPPGPAVATPAKAAPPPGSPAKF |
| | | RSLSRDPEAGRRQHAEERGRRNPKTGL |
| | ZNF106 | SAASFEVVRQCPTAEKPEQEHTPNKMPSLKSPLLPC |
| | | PATKSLSQKQDPKNISKNTKTNFFSP |
| | HNF1A | EEAFRHKLAMDTYSGPPPGPGPGPALPAHSSPGLPPP |
| | | ALSPSKVHGVRYGQPATSETAEVPS |
| | CLASP2 | NTGNGTQSSMGSPLTRPTPRSPANWSSPLTSPTNTSQ |
| | | NTLSPSAFDYDTENMNSEDIYSSLR |
| | KMT2B_0 | PVVSARSSRVIKTPRRFMDEDPPKPPKVEVSPVLRPP |
| | | ITTSPPVPQEPAPVPSPPRAPTPPS |
| | KMT2B_1 | IKTPRRFMDEDPPKPPKVEVSPVLRPPITTSPPVPQEP |
| | | APVPSPPRAPTPPSTPVPLPEKRR |
| | KMT2B_2 | EVSPVLRPPITTSPPVPQEPAPVPSPPRAPTPPSTPVPL |
| | | PEKRRSILREPTFRWTSLTRELP |
| 2373 | FLNC | KYVITIRFGGEHIPNSPFHVLACDPLPHEEEPSEVPQL |
| | | RQPYAPPRPGARPTHWATEEPVVP |
| 2374 | CIC_0 | GMFVWTNVEPRSVAVFPWHSLVPFLAPSQPDPSVQ |
| | | PSEAQQPASHPVASNQSKEPAESAAVA |
| | CIC_1 | PLVSPPFSVPVQNGAQPPSKIIQLTPVPVSTPSGLVPP |
| | | LSPATLPGPTSQPQKVLLPSSTRI |
| | CIC_2 | PTAPESELEGQPTPPAPPPLPETWTPTARSSPPLPPPA |
| | | EERTSAKGPETMASKFPSSSSDWR |
| | CIC_3 | FQARYADIFPSKVCLQLKIREVRQKIMQAATPTEQPP |
| | | GAEAPLPVPPPTGTAAAPAPTPSPA |
| | DCTN1_0 | GPSGSASAGELSSSEPSTPAQTPLAAPIIPTPVLTSPG |
| | | AVPPLPSPSKEEEGLRAQVRDLEE |
| | DCTN1_1 | ASAGELSSSEPSTPAQTPLAAPIIPTPVLTSPGAVPPLP |
| | | SPSKEEEGLRAQVRDLEEKLETL |
| 2375 | EPN1_0 | PPAADPWGGPAPTPASGDPWRPAAPAGPSVDPWGG |
| | | TPAPAAGEGPTPDPWGSSDGGVPVSGP |
| | EPN1_1 | PWGGPAPTPASGDPWRPAAPAGPSVDPWGGTPAPA |
| | | AGEGPTPDPWGSSDGGVPVSGPSASDP |
| | EPN1_2 | SGDPWRPAAPAGPSVDPWGGTPAPAAGEGPTPDPW |
| | | GSSDGGVPVSGPSASDPWTPAPAFSDP |
| 2376 | EPN1_3 | TPAPAAGEGPTPDPWGSSDGGVPVSGPSASDPWTPA |
| | | PAFSDPWGGSPAKPSTNGTTAAGGFD |
| 2377 | EPN1_4 | TPDPWGSSDGGVPVSGPSASDPWTPAPAFSDPWGG |
| | | SPAKPSTNGTTAAGGFDTEPDEFSDFD |
| | EPN1_5 | GSSDGGVPVSGPSASDPWTPAPAFSDPWGGSPAKPS |
| | | TNGTTAAGGFDTEPDEFSDFDRLRTA |
| | EPN1_6 | EVPARSPGAFDMSGVRGSLAEAVGSPPPAATPTPTP |
| | | PTRKTPESFLGPNAALVDLDSLVSRP |
| | EPN1_7 | DMSGVRGSLAEAVGSPPPAATPTPTPPTRKTPESFLG |
| | | PNAALVDLDSLVSRPGPTPPGAKAS |
| | CEBPE | TAMHLPPTLAAPGQPLRVLKAPLATAAPPCSPLLKA |
| | | PSPAGPLHKGKKAVNKDSLEYRLRRE |
| 2378 | KCNH2 | SSPESSEDEGPGRSSSPLRLVPFSSPRPPGEPPGGEPL |
| | | MEDCEKSSDTCNPLSGAFSGVSNI |
| | RFX4 | MKGEGSTAEVREEIILTEAAAPTPSPVPSFSPAKSATS |
| | | VEVPPPSSPVSNPSPEYTGLSTTG |
| 2379 | LPIN3_0 | PSTSVAGGVDPLGLPIQQTEAGADLQPDTEDPTLVG |
| | | PPLHTPETEESKTQSSGDMGLPPASK |
| | LPIN3_1 | PLGLPIQQTEAGADLQPDTEDPTLVGPPLHTPETEES |
| | | KTQSSGDMGLPPASKSWSWATLEVP |
| 2380 | RYR1 | ELPPEPEPEPEPELEPEKADAENGEKEEVPEPTPEPPK |
| | | KQAPPSPPPKKEEAGGEFWGELEV |
| | RAPGEF1 | SQSTELLPDATDEEVAPPKPPLPGIRVVDNSPPPALPP |
| | | KKRQSAPSPTRVAVVAPMSRATSG |
| | SAMD4A | AYSSPSTTPEARRREPQAPRQPSLMGPESQSPDCKD |
| | | GAAATGATATPSAGASGGLQPHQLSS |
| 2381 | C1orf198 | YGPEWARLPPAQQDEIIDRCLVGPRAPAPRDPGDSE |
| | | ELTRFPGLRGPTGQKVVRFGDEDLTW |
| 2382 | MANIC1 | VVAEIAGHAPAREQEPPPNPAPAAPAPGEDDPSSWA |
| | | SPRRRKGGLRRTRPTGPREEATAARG |
| | MAST4_0 | NPQQREGSSPKHQDHTTDPKLLTCLGQNLHSPDLAR |
| | | PRCPLPPEASPSREKPGLRESSERGP |
| | MAST4_1 | TTDPKLLTCLGQNLHSPDLARPRCPLPPEASPSREKP |
| | | GLRESSERGPPTARSERSAARADTC |
| | PRRC2C | QTHKPVQNPLQTTSQSSKQPPPSIRLPSAQTPNGTDY |
| | | VASGKSIQTPQSHGTLTAELWDNKV |
| 2383 | USP36 | QNGCIPPKLPSGSPSPKLSQTPTHMPTILDDPGKKVK |
| | | KPAPPQHFSPRTAQGLPGTSNSNSS |
| | PROP1 | MEAERRRQAEKPKKGRVGSNLLPERHPATGTPTTT |
| | | VDSSAPPCRRLPGAGGGRSRFSPQGGQ |
| | ARMC5_0 | RAQGGSFRSLRSWLISEGYATGPDDISPDWSPEQCPP |
| | | EPMEPASPAPTPTSLRAPRTQRTPG |
| 2384 | ARMC5_1 | RSWLISEGYATGPDDISPDWSPEQCPPEPMEPASPAP |
| | | TPTSLRAPRTQRTPGRSPAAAIEEP |
| | ARMC5_2 | ADSLSCLQDLVSPTVSPAVPQAVPMDLDSPSPCLYE |
| | | PLLGPAPVPAPDLHFLLDSGLQLPAQ |
| 2385 | CHD8_0 | KRKKYTEDLDIKITDDEEEEEVDVTGPIKPEPILPEPV |
| | | QEPDGETLPSMQFFVENPSEEDAA |
| 2386 | CHD8_1 | TEDLDIKITDDEEEEEVDVTGPIKPEPILPEPVQEPDG |
| | | ETLPSMQFFVENPSEEDAAIVDKV |
| 2387 | COL6A1 | LKGEKGEPGADGEAGRPGSSGPSGDEGQPGEPGPPG |
| | | EKGEAGDEGNPGPDGAPGERGGPGER |
| | CRYBG1_0 | SSPTKRKGRSRALEAVPAPPASGPRAPAKESPPKRVP |
| | | DPSPVTKGTAAESGEEAARAIPREL |
| | CRYBG1_1 | PTTVDTKDLPPTAMPKPQHTFSDSQSPAESSPGPSLS |
| | | LSAPAPGDVPKDTCVQSPISSFPCT |
| | DLAT_0 | IIVEKEADISAFADYRPTEVTDLKPQVPPPTPPPVAA |
| | | VPPTPQPLAPTPSAPCPATPAGPKG |
| | DLAT_1 | AFADYRPTEVTDLKPQVPPPTPPPVAAVPPTPQPLAP |
| | | TPSAPCPATPAGPKGRVFVSPLAKK |
| | DLAT_2 | TEVTDLKPQVPPPTPPPVAAVPPTPQPLAPTPSAPCP |
| | | ATPAGPKGRVFVSPLAKKLAVEKGI |
| | DLAT_3 | QVPPPTPPPVAAVPPTPQPLAPTPSAPCPATPAGPKG |
| | | RVFVSPLAKKLAVEKGIDLTQVKGT |
| | DENND2B_0 | ACRYPSHSSSRVLLKDRHPPAPSPQNPQDPSPDTSPP |
| | | TCPFKTASFGYLDRSPSACKRDAQK |
| | DENND2B_1 | NPVPKPKRTFEYEADKNPKSKPSNGLPPSPTPAAPPP |
| | | LPSTPAPPVTRRPKKDMRGHRKSQS |
| | DENND2B_2 | EYEADKNPKSKPSNGLPPSPTPAAPPPLPSTPAPPVT |
| | | RRPKKDMRGHRKSQSRKSFEFEDAS |
| 2388 | KATNIP | LRLSAVPTSMGDMPSAPATSPPVKCPPVHEEPSLIQQ |
| | | LENLMGRKICEPPGKTPSWLQPSPT |
| | PCDH12 | CEVGQSHKDVDKEAMMEAGWDPCLQAPFHLTPTL |
| | | YRTLRNQGNQGAPAESREVLQDTVNLLF |
| | SCARF2_0 | HTVEHGSPRTRDPTPRPPGLPEEATALAAPSPPRARA |
| | | RGRGPGLLEPTDAGGPPRSAPEAAS |
| | SCARF2_1 | LGRAEVALGAQGPREKPAPPQKAKRSVPPASPARAP |
| | | PATETPGPEKAATDLPAPETPRKKTP |
| | SCARF2_2 | QGPREKPAPPQKAKRSVPPASPARAPPATETPGPEK |
| | | AATDLPAPETPRKKTPIQKPPRKKSR |
| 2389 | IRAG1_0 | SIFGADAAEVPGTRGHSQQEAAMPHIPEDEEPPGEP |
| | | QAAQSPAGQGPPAAGVSCSPTPTIVL |
| | IRAG1_1 | PGTRGHSQQEAAMPHIPEDEEPPGEPQAAQSPAGQG |
| | | PPAAGVSCSPTPTIVLTGDATSPEGE |
| 2390 | AMER1 | WETAQMYPRPNMNLGYHPTTSPGHHGYMLLDPVR |
| | | SYPGLAPGELLTPQSDQQESAPNSDEGY |
| | CAMSAP3_0 | SLASPYLPEGTSKPLSDRPTKAPVYMPHPETPSKPSP |
| | | CLVGEASKPPAPSEGSPKAVASSPA |
| | CAMSAP3_1 | YLPEGTSKPLSDRPTKAPVYMPHPETPSKPSPCLVGE |
| | | ASKPPAPSEGSPKAVASSPAATNSE |
| 2391 | PIK3C2A | ALPSIYPSTYSKQAAFQNGFNPRMPTFPSTEPIYLSLP |
| | | GQSPYFSYPLTPATPFHPQGSLPI |
| 2392 | SP110_0 | AEGSSLHTPLALPPPQPPQPSCSPCAPRVSEPGTSSQQ |
| | | SDEILSESPSPSDPVLPLPALIQE |
| | SP110_1 | QPPQPSCSPCAPRVSEPGTSSQQSDEILSESPSPSDPV |
| | | LPLPALIQEGRSTSVTNDKLTSKM |
| | SP110_2 | DNLIPQIRDKEDPQEMPHSPLGSMPEIRDNSPEPNDP |
| | | EEPQEVSSTPSDKKGKKRKRCIWST |
| | COL6A2 | QKGKLGRIGPPGCKGDPGNRGPDGYPGEAGSPGER |
| | | GDQGGKGDPGRPGRRGPPGEIGAKGSK |
| | POLRIG | TCASAPQGTLRILEGPQQSLSGSPLQPIPASPPPQIPPG |
| | | LRPRFCAFGGNPPVTGPRSALAP |
| | USP54 | CSSSSSLPVIHDPSVFLLGPQLYLPQPQFLSPDVLMPT |
| | | MAGEPNRLPGTSRSVQQFLAMCDR |
| | FILIP1L | HTPGQPLHIKVTPDHVQNTATLEITSPTTESPHSYTS |
| | | TAVIPNCGTPKQRITILQNASITPV |
| 2393 | BRPF1 | PIMSSLRQRKRGRSPRPSSSSDSDSDKSTEDPPMDLP |
| | | ANGFSGGNQPVKKSFLVYRNDCSLP |
| | LITAF | GPYQAATGPSSAPSAPPSYEETVAVNSYYPTPPAPM |
| | | PGPTTGLVTGPDGKGMNPPSYYTQPA |
| | GLIS3 | HNPSSQLPPLTAVDAGAERFAPSAPSPHHISPRRVPA |
| | | PSSILQRTQPPYTQQPSGSHLKSYQ |
| | CPLANE1 | ISQAYGLMNELLSESVQLPTLPQKPLPNKPSPTQSSS |
| | | CQHCPSPRGENQHGHSFLINRPGKV |
| | CNOT2_0 | ALGLPMRGMSNNTPQLNRSLSQGTQLPSHVTPTTG |
| | | VPTMSLHTPPSPSRGILPMNPRNMMNH |
| | CNOT2_1 | LTFIRAAETDPGMVHLALGSDLTTLGLNLNSPENLY |
| | | PKFASPWASSPCRPQDIDFHVPSEYL |
| | CNOT2_2 | PGMVHLALGSDLTTLGLNLNSPENLYPKFASPWASS |
| | | PCRPQDIDFHVPSEYLTNIHIRDKLA |
| | CNOT2_3 | LALGSDLTTLGLNLNSPENLYPKFASPWASSPCRPQ |
| | | DIDFHVPSEYLTNIHIRDKLAAIKLG |
| | USP19_0 | LRKRQSQRWGGLEAPAARVGGAKVAVPTGPTPLDS |
| | | TPPGGAPHPLTGQEEARAVEKDKSKAR |
| | USP19_1 | SQRWGGLEAPAARVGGAKVAVPTGPTPLDSTPPGG |
| | | APHPLTGQEEARAVEKDKSKARSEDTG |
| | CNTFR | EFTIVKPDPPENVVARPVPSNPRRLEVTWQTPSTWP |
| | | DPESFPLKFFLRYRPLILDQWQHVEL |
| | MYO19 | QARYMADTFYTNAGCTLVALNPFKPVPQLYSPELM |
| | | REYHAAPQPQKLKPHVFTVGEQTYRNV |
| | NR4A1 | YGSPCSAPSPSTPSFQPPQLSPWDGSFGHFSPSQTYE |
| | | GLRAWTEQLPKASGPPQPPAFFSFS |
| | FAT4 | RSKSPQAMASHGSRPGSRLKQPIGQIPLESSPPVGLSI |
| | | EEVERLNTPRPRNPSICSADHGRS |
| 2394 | CC2D1B_0 | QLASVRRGRKINEDEIPPPVALGKRPLAPQEPANRSP |
| | | ETDPPAPPALESDNPSQPETSLPGI |
| | CC2D1B_1 | RRGRKINEDEIPPPVALGKRPLAPQEPANRSPETDPP |
| | | APPALESDNPSQPETSLPGISAQPV |
| | GRB7_0 | LDLSPPHLSSSPEDLCPAPGTPPGTPRPPDTPLPEEVK |
| | | RSQPLLIPTTGRKLREEERRATSL |
| | GRB7_1 | LIPTTGRKLREEERRATSLPSIPNPFPELCSPPSQSPIL |
| | | GGPSSARGLLPRDASRPHVVKVY |
| | GRB7_2 | GRKLREEERRATSLPSIPNPFPELCSPPSQSPILGGPSS |
| | | ARGLLPRDASRPHVVKVYSEDGA |
| 2395 | CLGN | FEVLVDQTVVNKGSLLEDVVPPIKPPKEIEDPNDKK |
| | | PEEWDERAKIPDPSAVKPEDWDESEP |
| | STPG1 | PGYYNPSDCTKVPKKTLFPKNPILNFSAQPSPLPPKP |
| | | PFPGPGQYEIVDYLGPRKHFISSAS |
| | TCOF1 | NPAAARAPSAKGTISAPGKVVTAAAQAKQRSPSKV |
| | | KPPVRNPQNSTVLARGPASVPSVGKAV |
| 2396 | ELF2_0 | PCVSTPEFIHAAMRPDVITETVVEVSTEESEPMDTSPI |
| | | PTSPDSHEPMKKKKVGRKPKTQQS |
| | ELF2_1 | PEFIHAAMRPDVITETVVEVSTEESEPMDTSPIPTSPD |
| | | SHEPMKKKKVGRKPKTQQSPISNG |
| | ELF2_2 | AAMRPDVITETVVEVSTEESEPMDTSPIPTSPDSHEP |
| | | MKKKKVGRKPKTQQSPISNGSPELG |
| 2397 | ELF2_3 | DVITETVVEVSTEESEPMDTSPIPTSPDSHEPMKKKK |
| | | VGRKPKTQQSPISNGSPELGIKKKP |
| 2398 | CLIP1 | PLCTSTASMVSSSPSTPSNIPQKPSQPAAKEPSATPPIS |
| | | NLTKTASESISNLSEAGSIKKGE |
| | BRD4_0 | GRGRKETGTAKPGVSTVPNTTQASTPPQTQTPQPNP |
| | | PPVQATPHPFPAVTPDLIVQTPVMTV |
| | BRD4_1 | QATPHPFPAVTPDLIVQTPVMTVVPPQPLQTPPPVPP |
| | | QPQPPPAPAPQPVQSHPPIIAATPQ |
| | BRD4_2 | PQQPSRPSNRAAALPPKPARPPAVSPALTQTPLLPQP |
| | | PMAQPPQVLLEDEEPPAPPLTSMQM |
| 2399 | SEPTIN4 | ELSKFVKDFSGNASCHPPEAKTWASRPQVPEPRPQA |
| | | PDLYDDDLEFRPPSRPQSSDNQQYFC |
| 2400 | MAP3K9_0 | GQLNQRVGIFPSNYVTPRSAFSSRCQPGGEDPSCYPP |
| | | IQLLEIDFAELTLEEIIGIGGFGKV |
| | MAP3K9_1 | DGALKPETLLASRSPSSNGLSPSPGAGMLKTPSPSRD |
| | | PGEFPRLPDPNVVFPPTPRRWNTQQ |
| 2401 | MAP3K9_2 | SSNGLSPSPGAGMLKTPSPSRDPGEFPRLPDPNVVFP |
| | | PTPRRWNTQQDSTLERPKTLEFLPR |
| | CBFA2T2 | RREENSFDRDTIAPEPPAKRVCTISPAPRHSPALTVPL |
| | | MNPGGQFHPTPPPLQHYTLEDIAT |
| 2402 | MYPN_0 | IAQLHVRGNEDLSNNGSLHSANSTTNLAAIEPQPSPP |
| | | HSEPPSVEQPPKPKLEGVLVNHNEP |
| | MYPN_1 | SEASSEAGVVTTRQTRPDSFQERFNGQATKTPEPSSP |
| | | VKEPPPVLAKPKLDSTQLQQLHNQV |
| | MYPN_2 | LLVSHPSVQTKSPGGLSIQNEPLPPGPTEPTPPPFTFSI |
| | | PSGNQFQPRCVSPIPVSPTSRIQ |
| | PTCHD3 | SATGPQWYQESQESESEGKQPPPGPLAPPKSPEPSGP |
| | | LASEQDAPLPEGDDAPPRPSMLDDA |
| | KDM6B | PPAPPSSCHQNTSGSFRRPESPRPRVSFPKTPEVGPGP |
| | | PPGPLSKAPQPVPPGVGELPARGP |
| | C2CD5 | GESGLVVRAIGTACTLDKLSSPAAFLPACNSPSKEM |
| | | KEIPFNEDPNPNTHSSGPSTPLKNQT |
| | SEC16B | GTTTENTFYQDFSGCQGYSEAPGYRSALWLTPEQTC |
| | | LLQPSPQQPFPLQPGSYPAGGGAGQT |
| | ARAP1_0 | AHTSPAPAPRPTPRPVPMKRHIFRSPPVPATPPEPLPT |
| | | TTEDEGLPAAPPIPPRRSCLPPTC |
| 2403 | ARAP1_1 | GPPRLLVSLPTKEEESLLPSLSSPPQPQSEEPLSTLPQ |
| | | GPPQPPSPPPCPPEIPPKPVRLFP |
| | ARAP1_2 | NGGWHTSSLSLSLPSTIAAPHPMDGPPGGSTPVTPVI |
| | | KAGWLDKNPPQGSYIYQKRWVRLDT |
| | TRAPPC12 | EGDAGDLGRVRDEAEPGGEGDPGPEPAGTPSPSGEA |
| | | DGDCAPEDAAPSSGGAPRQDAAREVP |
| | ACACA | ADVNLPAAQLQIAMGIPLYRIKDIRMMYGVSPWGD |
| | | SPIDFEDSAHVPCPRGHVIAARITSEN |
| | UBP1_0 | EDAVEHEQKKSSKRTLPADYGDSLAKRGSCSPWPD |
| | | APTAYVNNSPSPAPTFTSPQQSTCSVP |
| | UBP1_1 | LPADYGDSLAKRGSCSPWPDAPTAYVNNSPSPAPTF |
| | | TSPQQSTCSVPDSNSSSPNHQGDGAS |
| | DENND1A | AWSGSTLPSRPATPNVATPFTPQFSFPPAGTPTPFPQP |
| | | PLNPFVPSMPAAPPTLPLVSTPAG |
| | FAM193A_0 | GIMDPPVTDDIHIHQLPLQVDPAPDYLAERSPPSVSS |
| | | ASSGSGSSSPITIQQHPRLILTDSG |
| | FAM193A_1 | SSEADDEEADGESSGEPPGAPKEDGVLGSRSPRTEES |
| | | KADSPPPSYPTQQAEQAPNTCECHV |
| | FAM193A_2 | LHLYPHIHGHVPLHTVPHLPRPLIHPTLYATPPFTHS |
| | | KALPPAPVQNHTNKHQVFNASLQDH |
| | FAM193A_3 | FHGISKEDHRHSAPAAPRNSPTGLAPLPALSPAALSP |
| | | AALSPASTPHLANLAAPSFPKTATT |
| | FAM193A_4 | HSAPAAPRNSPTGLAPLPALSPAALSPAALSPASTPH |
| | | LANLAAPSFPKTATTTPGFVDTRKS |
| | SCYL3 | LNQLVFAEPVAVKSFLPYLLGPKKDHAQGETPCLLS |
| | | PALFQSRVIPVLLQLFEVHEEHVRMV |
| 2404 | YY1AP1 | EEASRSAAATNPGSRLTRWPPPDKREGSAVDPGKR |
| | | RSLAATPSSSLPCTLIALGLRHEKEAN |
| 2405 | MGRN1 | PFKKSKPHPASLASKKPKRETNSDSVPPGYEPISLLE |
| | | ALNGLRAVSPAIPSAPLYEEITYSG |
| | QRICH1_0 | LTVHQPTEQPIQVQVQIQGQAPQSAAPSIQTPSLQSP |
| | | SPSQLQAAQIQVQHVQAAQQIQAAE |
| | QRICH1_1 | PTEQPIQVQVQIQGQAPQSAAPSIQTPSLQSPSPSQLQ |
| | | AAQIQVQHVQAAQQIQAAEIPEEH |
| | TFPT_0 | TIVLEDEGSQGTDAPTPGNAENEPPEKETLSPPRRTP |
| | | APPEPGSPAPGEGPSGRKRRRVPRD |
| | TFPT_1 | DEGSQGTDAPTPGNAENEPPEKETLSPPRRTPAPPEP |
| | | GSPAPGEGPSGRKRRRVPRDGRRAG |
| 2406 | TFPT_2 | GTDAPTPGNAENEPPEKETLSPPRRTPAPPEPGSPAP |
| | | GEGPSGRKRRRVPRDGRRAGNALTP |
| | CXXC1 | GGPNKIRQKCRLRQCQLRARESYKYFPSSLSPVTPSE |
| | | SLPRPRRPLPTQQQPQPSQKLGRIR |
| 2407 | MZF1 | RQRSNLLQHQRIHGDPPGPGAKPPAPPGAPEPPGPFP |
| | | CSECRESFARRAVLLEHQAVHTGDK |
| | GORASP1 | PSYHKKPPGTPPPSALPLGAPPPDALPPGPTPEDSPSL |
| | | ETGSRQSDYMEALLQAPGSSMEDP |
| 2408 | PRR14_0 | QRAEPMRIVRQPTPPPGDLEPPFQPSALPADPLESPPT |
| | | APDPALELPSTPPPSSLLRPRLSP |
| 2409 | PRR14_1 | QPTPPPGDLEPPFQPSALPADPLESPPTAPDPALELPS |
| | | TPPPSSLLRPRLSPWGLAPLFRSV |
| | PRR14_2 | DPLESPPTAPDPALELPSTPPPSSLLRPRLSPWGLAPL |
| | | FRSVRSKLESFADIFLTPNKTPQP |
| | CRYZL2P-SEC16B | GTTTENTFYQDFSGCQGYSEAPGYRSALWLTPEQTC |
| | | LLQPSPQQPFPLQPGSYPAGGGAGQT |
| | NTRK1 | PFGQASASIMAAFMDNPFEFNPEDPIPVSFSPVDTNS |
| | | TSGDPVEKKDETPFGVSVAVGLAVF |
| 2410 | DOK1 | KPLYWDLYEHAQQQLLKAKLTDPKEDPIYDEPEGL |
| | | APVPPQGLYDLPREPKDAWWCQARVKE |
| | HMGXB3 | PGADVPTPSEGTSTSSPLPAPKKPTGADLLTPGSRAP |
| | | ELKGRARGKPSLLAAARPMRAILPA |
| | HMX2 | KAPACFCPDQHGPKEQGPKHHPPIPFPCLGTPKGSG |
| | | GSGPGGLERTPFLSPSHSDFKEEKER |
| 2411 | THADA | CNMGEKFLLLAMKENHPECFCKILKILHCMDPGEW |
| | | LPQTEHCVHLTPKEFLIWTMDIASNER |
| | MGA | KPLILSRKKDQATENTSPLNTPHTSANLVMTPQGQL |
| | | LTLKGPLFSGPVVAVSPDLLESDLKP |
| | FBF1 | LFPASPTREAHRESSVPVTPSVPPPASQHSTPAGLPPS |
| | | RAKPPTEGAGSPAKASQASKLRAS |
| | SULT1A1 | KCHRAPIFMRVPFLEFKAPGIPSGMETLKDTPAPRLL |
| | | KTHLPLALLPQTLLDQKVKVVYVAR |
| | KAT14 | SSSDRTPLTSPSPSPSLDFSAPGTPASHSATPSLLSEA |
| | | DLIPDVMPPQALFHDDDEMEGDGV |
| | ELK1_0 | PERTPGSGSGSGLQAPGPALTPSLLPTHTLTPVLLTPS |
| | | SLPPSIHFWSTLSPIAPRSPAKLS |
| | ELK1_1 | GSGSGSGLQAPGPALTPSLLPTHTLTPVLLTPSSLPPS |
| | | IHFWSTLSPIAPRSPAKLSFQFPS |
| | DAG1_0 | IHATPTPVTAIGPPTTAIQEPPSRIVPTPTSPAIAPPTET |
| | | MAPPVRDPVPGKPTVTIRTRGA |
| 2412 | DAG1_1 | LGPIQPTRVSEAGTTVPGQIRPTMTIPGYVEPTAVAT |
| | | PPTTTTKKPRVSTPKPATPSTDSTT |
| 2413 | PASK | EHYAASDRESPGHVPSTLDAGPEDTCPSAEEPRLNV |
| | | QVTSTPVIVMRGAAGLQREIQEGAYS |
| 2414 | MOV10 | CMEPESLVAIAGLMEVKETGDPGGQLVLAGDPRQL |
| | | GPVLRSPLTQKHGLGYSLLERLLTYNS |
| | GLIS1 | PLDATTSSHHHLSPLPMAESTRDGLGPGLLSPIVSPL |
| | | KGLGPPPLPPSSQSHSPGGQPFPTL |
| | PRDM2 | SSASPHPCPSPLSNATAQSPLPILSPTVSPSPSPIPPVEP |
| | | LMSAASPGPPTLSSSSSSSSSS |
| | POU2F2_0 | WFCNRRQKEKRINPCSAAPMLPSPGKPASYSPHMV |
| | | TPQGGAGTLPLSQASSSLSTTVTTLSS |
| | POU2F2_1 | RQKEKRINPCSAAPMLPSPGKPASYSPHMVTPQGGA |
| | | GTLPLSQASSSLSTTVTTLSSAVGTL |
| | FOXN1_0 | KHAGFSCSSFVSDGPPERTPSLPPHSPRIASPGPEQVQ |
| | | GHCPAGPGPGPFRLSPSDKYPGFG |
| | FOXN1_1 | APGPIPGKNPLQDLLMGHTPSCYGQTYLHLSPGLAP |
| | | PGPPQPLFPQPDGHLELRAQPGTPQD |
| | RIMS1 | DVELESESVSEKGDLDYYWLDPATWHSRETSPISSH |
| | | PVTWQPSKEGDRLIGRVILNKRTTMP |
| | MED12L | LYHTHPMPKPRSYYLQPLPLPPEEEEEEPTSPVSQEP |
| | | ERKSAELSDQGKTTTDEEKKTKGRK |
| | REPIN1 | HKRSEGSAQAAPGPGSPQLPAGPQESAAEPTPAVPL |
| | | KPAQEPPPGAPPEHPQDPIEAPPSLY |
| 2415 | WNK2_0 | AYQQPTAAPGLPVGSVPAPACPPSLQQHFPDPAMSF |
| | | APVLPPPSTPMPTGPGQPAPPGQQPP |
| | WNK2_1 | SVPAPACPPSLQQHFPDPAMSFAPVLPPPSTPMPTGP |
| | | GQPAPPGQQPPPLAQPTPLPQVLAP |
| | WNK2_2 | TPLAGIDGLPPALPDLPTATVPPVPPPQYFSPAVILPS |
| | | LAAPLPPASPALPLQAVKLPHPPG |
| 2416 | WNK2_3 | TRPPQPVLPPQPMLPPQPVLPPQPALPVRPEPLQPHL |
| | | PEQAAPAATPGSQILLGHPAPYAVD |
| | WNK2_4 | VSASVQSVPTQTATLLPPANPPLPGGPGIASPCPTVQ |
| | | LTVEPVQEEQASQDKPPGLPQSCES |
| 2417 | WNK2_5 | QTATLLPPANPPLPGGPGIASPCPTVQLTVEPVQEEQ |
| | | ASQDKPPGLPQSCESYGGSDVTSGK |
| 2418 | WNK2_6 | DRDGRQVASDSHVVPSVPQDVPAFVRPARVEPTDR |
| | | DGGEAGESSAEPPPSDMGTVGGQASHP |
| 2419 | ZFR2_0 | EERMRKQRHLAEERLEQLRRWHAERRRLEEEPPQD |
| | | VPPHAPPDWAQPLLMGRPESPASAPLQ |
| 2420 | ZFR2_1 | ANIVISSCEEPRMQVTISVTSPLMREDPSTDPGVEEP |
| | | QADAGDVLSPKKCLESLAALRHARW |
| 2421 | ZFR2_2 | SSCEEPRMQVTISVTSPLMREDPSTDPGVEEPQADA |
| | | GDVLSPKKCLESLAALRHARWFQARA |
| | GTF3C2_0 | TPMPKKRGRKSKAELLLLKLSKDLDRPESQSPKRPP |
| | | EDFETPSGERPRRRAAQVALLYLQEL |
| | GTF3C2_1 | SKAELLLLKLSKDLDRPESQSPKRPPEDFETPSGERP |
| | | RRRAAQVALLYLQELAEELSTALPA |
| | BTBD18 | TQDSPQIPDPGGDFQEPSGTQPFSSNEQEMSPTRTEL |
| | | CQDSPMCTKLQDILVSASHSPDHPV |
| 2422 | SPOCD1 | SCGDNIFQKALSQTPMPAPEMPKTRELSPTEPQDRV |
| | | PPSGLHVPAAPTKALPCLPPWEGVLD |
| | STXBP5 | TEVIPMLEVRLLYEINDVETPEGEQPPPLPTPVGGSN |
| | | PQPIPPQSHPSTSSSSSDGLRDNVP |
| | CNOT1_0 | CSNVMNKARQPPPGVMPKGRPPSASSLDAISPVQID |
| | | PLAGMTSLSIGGSAAPHTQSMQGFPP |
| 2423 | CNOT1_1 | NKARQPPPGVMPKGRPPSASSLDAISPVQIDPLAGM |
| | | TSLSIGGSAAPHTQSMQGFPPNLGSA |
| 2424 | CNOT4_0 | CGYQICRFCWHRIRTDENGLCPACRKPYPEDPAVYK |
| | | PLSQEELQRIKNEKKQKQNERKQKIS |
| | CNOT4_1 | EGAVTESQSLFSDNFRHPNPIPSGLPPFPSSPQTSSDW |
| | | PTAPEPQSLFTSETIPVSSSTDWQ |
| 2425 | CNOT4_2 | DNFRHPNPIPSGLPPFPSSPQTSSDWPTAPEPQSLFTS |
| | | ETIPVSSSTDWQAAFGFGSSKQPE |
| | FETUB | SQAPATGSENSAVNQKPTNLPKVEESQQKNTPPTDS |
| | | PSKAGPRGSVQYLPDLDDKNSQEKGP |
| | BCL11A_0 | AMEPPAMDFSRRLRELAGNTSSPPLSPGRPSPMQRL |
| | | LQPFQPGSKPPFLATPPLPPLQSAPP |
| | BCL11A_1 | SSPPLSPGRPSPMQRLLQPFQPGSKPPFLATPPLPPLQ |
| | | SAPPPSQPPVKSKSCEFCGKTFKF |
| 2426 | KDM3B_0 | LKGDRGEVDSNGSDGGEASRGPWKGGNASGEPGL |
| | | DQRAKQPPSTFVPQINRNIRFATYTKEN |
| | KDM3B_1 | GPSLSAMGNGRSSSPTSSLTQPIEMPTLSSSPTEERPT |
| | | VGPGQQDNPLLKTFSNVFGRHSGG |
| 2427 | BAHD1_0 | ENVAGPRSADEADELPPDLPKPPSPAPSSEDPGLAQP |
| | | RKRRLASLNAEALNNLLLEREDTSS |
| 2428 | BAHD1_1 | LEFPLPEAGHPASPAHPLLGCPVPSVPPAAEPVPHLQ |
| | | TPTSEPQTVARACPQSAKPPSGSKS |
| 2429 | PNPLA2 | LLLGLFCTNVAFPPEALRMRAPADPAPAPADPASPQ |
| | | HQLAGPAPLLSTPAPEARPVIGALGL |
| | RBM10 | SQSYTIMSPAVLKSELQSPTHPSSALPPATSPTAQES |
| | | YSQYPVPDVSTYQYDETSGYYYDPQ |
| | KIF20A | KKRLGTNQENQQPNQQPPGKKPFLRNLLPRTPTCQS |
| | | STDCSPYARILRSRRSPLLKSGPFGK |
| 2430 | OSGIN1 | KVFGVSLVLVLIGSHPDLSFLPGAGADFAVDPDQPL |
| | | SAKRNPIDVDPFTYQSTRQEGLYAMG |
| | DGKZ_0 | YVTEIAQDEIYILDPELLGASARPDLPTPTSPLPTSPC |
| | | SPTPRSLQGDAAPPQGEELIEAAK |
| | DGKZ_1 | AQDEIYILDPELLGASARPDLPTPTSPLPTSPCSPTPRS |
| | | LQGDAAPPQGEELIEAAKRNDFC |
| | DGKZ_2 | YILDPELLGASARPDLPTPTSPLPTSPCSPTPRSLQGD |
| | | AAPPQGEELIEAAKRNDFCKLQEL |
| | FOXF2 | PVPSSPAMASAIECHSPYTSPAAHWSSPGASPYLKQP |
| | | PALTPSSNPAASAGLHSSMSSYSLE |
| | HSPG2 | NKVGSAEAFAQLLVQGPPGSLPATSIPAGSTPTVQV |
| | | TPQLETKSIGASVEFHCAVPSDRGTQ |
| 2431 | MIA3_0 | ERAIAEEKREAANLRHKLLELTQKMAMLQEEPVIV |
| | | KPMPGKPNTQNPPRRGPLSQNGSFGPS |
| | MIA3_1 | GSSPTRVLDEGKVNMAPKGPPPFPGVPLMSTPMGG |
| | | PVPPPIRYGPPPQLCGPFGPRPLPPPF |
| | CREB3L2_0 | PTPPSSHGSDSEGSLSPNPRLHPFSLPQTHSPSRAAPR |
| | | APSALSSSPLLTAPHKLQGSGPLV |
| | CREB3L2_1 | SPNPRLHPFSLPQTHSPSRAAPRAPSALSSSPLLTAPH |
| | | KLQGSGPLVLTEEEKRTLIAEGYP |
| | NFATC1_0 | PQRSTLMPAAPGVSPKLHDLSPAAYTKGVASPGHC |
| | | HLGLPQPAGEAPAVQDVPRPVATHPGS |
| | NFATC1_1 | PGHCHLGLPQPAGEAPAVQDVPRPVATHPGSPGQPP |
| | | PALLPQQVSAPPSSSCPPGLEHSLCP |
| | PDE5A | PVCKEGIRGHTESCSCPLQQSPRADNSAPGTPTRKIS |
| | | ASEFDRPLRPIVVKDSEGTVSFLSD |
| | PRDM15 | ELRVWYAAFYAKKMDKPMLKQAGSGVHAAGTPE |
| | | NSAPVESEPSQWACKVCSATFLELQLLNE |
| | MYBL2_0 | VTTPLHRDKTPLHQKHAAFVTPDQKYSMDNTPHTP |
| | | TPFKNALEKYGPLKPLPQTPHLEEDLK |
| | MYBL2_1 | HRDKTPLHQKHAAFVTPDQKYSMDNTPHTPTPFKN |
| | | ALEKYGPLKPLPQTPHLEEDLKEVLRS |
| | ZYX | YVPPPVATPFSSKSSTKPAAGGTAPLPPWKSPSSSQP |
| | | LPQVPAPAQSQTQFHVQPQPQPKPQ |
| | FCMR | ARGADAAGTGEAPVPGPGAPLPPAPLQVSESPWLH |
| | | APSLKTSCEYVSLYHQPAAMMEDSDSD |
| | ATG12_0 | MAEEPQSVLQLPTSIAAGGEGLTDVSPETTTPEPPSS |
| | | AAVSPGTEEPAGDTKKKIDILLKAV |
| | ATG12_1 | LPTSIAAGGEGLTDVSPETTTPEPPSSAAVSPGTEEPA |
| | | GDTKKKIDILLKAVGDTPIMKTKK |
| 2432 | DMRT3 | QLRSQYVSPFPSNSTSVFRSSPVLPARATEDPRISIPD |
| | | DGCPFVSKQSIYTEDDYDERSDSS |
| | DLGAP2 | LCSGHTCGLAPPEDCEHLHHGPDARPPYLLSPADSC |
| | | PGGRHRCSPRSSVHSECVMMPVVLGD |
| | DNM3_0 | LGIIGDISTATVSTPAPPPVDDSWIQHSRRSPPPSPTT |
| | | QRRPTLSAPLARPTSGRGPAPAIP |
| | DNM3_1 | PPSPTTQRRPTLSAPLARPTSGRGPAPAIPSPGPHSGA |
| | | PPVPFRPGPLPPFPSSSDSFGAPP |
| | KLF16 | LAASILADLRGGPGAAPGGASPASSSSAASSPSSGRA |
| | | PGAAPSAAAKSHRCPFPDCAKAYYK |
| | WNT6 | TQACSMGELLQCGCQAPRGRAPPRPSGLPGTPGPPG |
| | | PAGSPEGSAAWEWGGCGDDVDFGDEK |
| | MUC16_0 | MTYTEKSEVSSSIHPRPETSAPGAETTLTSTPGNRAIS |
| | | LTLPFSSIPVEEVISTGITSGPDI |
| | MUC16_1 | RGPGDMSWQSSPSLENPSSLPSLLSLPATTSPPPISST |
| | | LPVTISSSPLPVTSLLTSSPVTTT |
| | MUC16_2 | PEDVSWPSPLSVEKNSPPSSLVSSSSVTSPSPLYSTPS |
| | | GSSHSSPVPVTSLFTSIMMKATDM |
| 2423 | KCNQ1 | APGPAPPASPAAPAAPPVASDLGPRPPVSLDPRVSIY |
| | | STRRPVLARTHVQGRVYNFLERPTG |
| 2424 | BCAR1_0 | PSVSKDVPDGPLLREETYDVPPAFAKAKPFDPARTP |
| | | LVLAAPPPDSPPAEDVYDVPPPAPDL |
| | BCAR1_1 | ETYDVPPAFAKAKPFDPARTPLVLAAPPPDSPPAED |
| | | VYDVPPPAPDLYDVPPGLRRPGPGTL |
| | FOXO4 | APGPSSLVPTLSMIAPPPVMASAPIPKALGTPVLTPPT |
| | | EAASQDRMPQDLDLDMYMENLECD |
| | AKTIS1 | RCLHDIALAHRAATAARPPAPPPAPQPPSPTPSPPRP |
| | | TLAREDNEEDEDEPTETETSGEQLG |
| 2425 | COL5A2_0 | SVGPVGPRGPQGLQGQQGGAGPTGPPGEPGDPGPM |
| | | GPIGSRGPEGPPGKPGEDGEPGRNGNP |
| | COL5A2_1 | AIGTDGTPGAKGPTGSPGTSGPPGSAGPPGSPGPQGS |
| | | TGPQGIRGQPGDPGVPGFKGEAGPK |
| 2426 | COL5A2_2 | PPGPTGFQGLPGPPGPPGEGGKPGDQGVPGDPGAVG |
| | | PLGPRGERGNPGERGEPGITGLPGEK |
| 2427 | COL5A2_3 | TPGKVGPTGATGDKGPPGPVGPPGSNGPVGEPGPEG |
| | | PAGNDGTPGRDGAVGERGDRGDPGPA |
| | CTC1_0 | SYLPPARWNSSGEGHLELWDAPVPVFPLTISPGPVTP |
| | | IPVLYPESASCLLRLRNKLRGVQRN |
| | CTC1_1 | ARWNSSGEGHLELWDAPVPVFPLTISPGPVTPIPVLY |
| | | PESASCLLRLRNKLRGVQRNLAGSL |
| | SH2D6 | PLSLAPAHLPGTEEDSLYLDHSGPLGPSKPSPPLPQP |
| | | TMLKGAVSLPVAGKQGPIFGRREQG |
| | KSR1 | DSSSNPSSTTSSTPSSPAPFPTSSNPSSATTPPNPSPGQ |
| | | RDSRFNFPAAYFIHHRQQFIFPV |
| | Clorf127_0 | AAPVLWTVESFFQCVGSGTESPASTAALRTTPSPPSP |
| | | GPETPPAGVPPAASSQVWAAGPAAQ |
| | Clorf127_1 | WTVESFFQCVGSGTESPASTAALRTTPSPPSPGPETP |
| | | PAGVPPAASSQVWAAGPAAQEWLSR |
| | Clorf127_2 | FFQCVGSGTESPASTAALRTTPSPPSPGPETPPAGVPP |
| | | AASSQVWAAGPAAQEWLSRDLLHR |
| | Clorf127_3 | QTSASILPRVVQAQRGPQPPPGEAGIPGHPTPPATLP |
| | | SEPVEGVQASPWRPRPVLPTHPALT |
| | Clorf127_4 | GVQASPWRPRPVLPTHPALTLPVSSDASSPSPPAPRP |
| | | ERPESLLVSGPSVTLTEGLGTVRPE |
| | Clorf127_5 | GHMDLSSSEPSQDIEGPGLSILPARDATFSTPSVRQP |
| | | DPSAWLSSGPELTGMPRVRLAAPLA |
| | C2CD4D_0 | AEPAARWAPSGLFSKRRAPGPPTSACPNVLTPDRIP |
| | | QFFIPPRLPDPGGAVPAARRHVAGRG |
| 2428 | C2CD4D_1 | RRAPGPPTSACPNVLTPDRIPQFFIPPRLPDPGGAVPA |
| | | ARRHVAGRGLPATCSLPHLAGREG |
| | C2CD4D_2 | SDTASSPDSSPFGSPRPGLGRRRVSRPHSLSPEKASS |
| | | ADTSPHSPRRAGPPTPPLFHLDFLC |
| 2429 | MESP1 | GLGLVSAVRAGASWGSPPACPGARAAPEPRDPPAL |
| | | FAEAACPEGQAMEPSPPSPLLPGDVLA |
| 2430 | PCF11 | DPAWPIKPLPPNVNTSSIHVNPKFLNKSPEEPSTPGT |
| | | VVSSPSISTPPIVPDIQKNLTQEQL |
| | LHX6 | TLQKLADMTGLSRRVIQVWFQNCRARHKKHTPQHP |
| | | VPPSGAPPSRLPSALSDDIHYTPFSSP |
| | FRMD1 | MAVPPRGRGIDPARTNPDTFPPSGARCMEPSPERPA |
| | | CSQQEPTLGMDAMASEHRDVLVLLPS |
| 2431 | SPHK2_0 | GSARFTLGTVLGLATLHTYRGRLSYLPATVEPASPT |
| | | PAHSLPRAKSELTLTPDPAPPMAHSP |
| | SPHK2_1 | TLGTVLGLATLHTYRGRLSYLPATVEPASPTPAHSL |
| | | PRAKSELTLTPDPAPPMAHSPLHRSV |
| | SPHK2_2 | GRLSYLPATVEPASPTPAHSLPRAKSELTLTPDPAPP |
| | | MAHSPLHRSVSDLPLPLPQPALASP |
| | SPHK2_3 | EPASPTPAHSLPRAKSELTLTPDPAPPMAHSPLHRSV |
| | | SDLPLPLPQPALASPGSPEPLPILS |
| | SPHK2_4 | AGDWGGAGDAPLSPDPLLSSPPGSPKAALHSPVSEG |
| | | APVIPPSSGLPLPTPDARVGASTCGP |
| | LACTB | GAAPAQSPAAPDPEASPLAEPPQEQSLAPWSPQTPA |
| | | PPCSRCFARAIESSRDLLHRIKDEVG |
| | SMAD2 | YISEDGETSDQQLNQSMDTGSPAELSPTTLSPVNHSL |
| | | DLQPVTYSEPAFWCSIAYYELNQRV |
| | TET3_0 | AKEKNISLQTAIAIEALTQLSSALPQPSHSTPQASCPL |
| | | PEALSPPAPFRSPQSYLRAPSWPV |
| | TET3_1 | KRSLFLEQVHDTSFPAPSEPSAPGWWPPPSSPVPRLP |
| | | DRPPKEKKKKLPTPAGGPVGTEKAA |
| 2432 | COL1A1_0 | VPGPMGPSGPRGLPGPPGAPGPQGFQGPPGEPGEPG |
| | | ASGPMGPRGPPGPPGKNGDDGEAGKP |
| 2433 | COL1A1_1 | PMGPSGPRGLPGPPGAPGPQGFQGPPGEPGEPGASG |
| | | PMGPRGPPGPPGKNGDDGEAGKPGRP |
| | COL1A1_2 | PPGERGGPGSRGFPGADGVAGPKGPAGERGSPGPA |
| | | GPKGSPGEAGRPGEAGLPGAKGLTGSP |
| | PER1_0 | RDFTQEKSVFCRIRGGPDRDPGPRYQPFRLTPYVTKI |
| | | RVSDGAPAQPCCLLIAERIHSGYEA |
| | PER1_1 | HQNPRAEAPCYVSHPSPVPPSTPWPTPPATTPFPAVV |
| | | QPYPLPVFSPRGGPQPLPPAPTSVP |
| | PER1_2 | LPNYLFPTPSSYPYGALQTPAEGPPTPASHSPSPSLPA |
| | | LAPSPPHRPDSPLFNSRCSSPLQL |
| | CARMIL1 | ENRFGLGTPEKNTKAEPKAEAGSRSRSSSSTPTSPKP |
| | | LLQSPKPSLAARPVIPQKPRTASRP |
| 2434 | WNT10A | PEFRTVGALLRSRFHRATLIRPHNRNGGQLEPGPAG |
| | | APSPAPGAPGPRRRASPADLVYFEKS |
| | CDCA8 | VGRLEVSMVKPTPGLTPRFDSRVFKTPGLRTPAAGE |
| | | RIYNISGNGSPLADSKEIFLTVPVGG |
| | AMPH_0 | AFTIQGAPSDSGPLRIAKTPSPPEEPSPLPSPTASPNHT |
| | | LAPASPAPARPRSPSQTRKGPPV |
| | AMPH_1 | LRIAKTPSPPEEPSPLPSPTASPNHTLAPASPAPARPR |
| | | SPSQTRKGPPVPPLPKVTPTKELQ |
| | POGZ_0 | QKKGKSLDSEPSVPSAAKPPSPEKTAPVASTPSSTPIP |
| | | ALSPPTKVPEPNENVGDAVQTKLI |
| | POGZ_1 | PSVPSAAKPPSPEKTAPVASTPSSTPIPALSPPTKVPEP |
| | | NENVGDAVQTKLIMLVDDFYYGR |
| | POGZ_2 | AGATPAEPEELLTPLAPALPSPASTATPPPTPTHPQA |
| | | LALPPLATEGAECLNVDDQDEGSPV |
| | NRIP1 | YARTSVIESPSTNRTTPVSTPPLLTSSKAGSPINLSQH |
| | | SLVIKWNSPPYVCSTQSEKLTNTA |
| | CHRNA4 | ATSGTQSLHPPSPSFCVPLDVPAEPGPSCKSPSDQLPP |
| | | QQPLEAEKASPHPSPGPCRPPHGT |
| 2435 | IRF5 | PPTLQPPTLRPPTLQPPTLQPPVVLGPPAPDPSPLAPP |
| | | PGNPAGFRELLSEVLEPGPLPASL |
| | PIK3R2 | RPRGPRPLPARPRDGAPEPGLTLPDLPEQFSPPDVAP |
| | | PLLVKLVEAIERTGLDSESHYRPEL |
| | ADAM17 | LSLFHPSNVEMLSSMDSASVRIIKPFPAPQTPGRLQP |
| | | APVIPSAPAAPKLDHQRMDTIQEDP |
| | PXN_0 | LLLELNAVQHNPPGFPADEANSSPPLPGALSPLYGV |
| | | PETNSPLGGKAGPLTKEKPKRNGGRG |
| | PXN_1 | NPPGFPADEANSSPPLPGALSPLYGVPETNSPLGGKA |
| | | GPLTKEKPKRNGGRGLEDVRPSVES |
| 2436 | UBR5_0 | AGLGRHEAGASSSDHQDPVSPPIAPPSWVPDPPAMD |
| | | PDGDIDFILAPAVGSLTTAATGTGQG |
| 2437 | UBR5_1 | HEAGASSSDHQDPVSPPIAPPSWVPDPPAMDPDGDI |
| | | DFILAPAVGSLTTAATGTGQGPSTST |
| | SNAI2 | THTVIISPYLYESYSMPVIPQPEILSSGAYSPITVWTT |
| | | AAPFHAQLPNGLSPLSGYSSSLGR |
| | IRS2 | NSASVENVSLRKSSEGGVGVGPGGGDEPPTSPRQLQ |
| | | PAPPLAPQGRPWTPGQPGGLVGCPGS |
| | USP10_0 | DGTGSASGTLPVSQPKSWASLFHDSKPSSSSPVAYV |
| | | ETKYSPPAISPLVSEKQVEVKEGLVP |
| | USP10_1 | PVSQPKSWASLFHDSKPSSSSPVAYVETKYSPPAISP |
| | | LVSEKQVEVKEGLVPVSEDPVAIKI |
| | USP10_2 | KSWASLFHDSKPSSSSPVAYVETKYSPPAISPLVSEK |
| | | QVEVKEGLVPVSEDPVAIKIAELLE |
| | GFI1B | EPELEQDQNLARMAPAPEGPIVLSRPQDGDSPLSDSP |
| | | PFYKPSFSWDTLATTYGHSYRQAPS |
| | LPA | PVTESSVLTTPTVAPVPSTEAPSEQAPPEKSPVVQDC |
| | | YHGDGRSYRGISSTTVTGRTCQSWS |
| | TNKS1BP1_0 | QTPEASQASPCPAVTPSAPSAALPDEGSRHTPSPGLP |
| | | AEGAPEAPRPSSPPPEVLEPHSLDQ |
| 2438 | TNKS1BP1_1 | EGSRHTPSPGLPAEGAPEAPRPSSPPPEVLEPHSLDQP |
| | | PATSPRPLIEVGELLDLTRTFPSG |
| 2439 | VCP | VINQILTEMDGMSTKKNVFIIGATNRPDIIDPAILRPG |
| | | RLDQLIYIPLPDEKSRVAILKANL |
| 2440 | CDKL5 | PLSQASGGSSNIRQEPAPKGRPALQLPGQMDPGWH |
| | | VSSVTRSATEGPSYSEQLGAKSGPNGH |
| 2441 | CYP46A1 | ETLIDGVRVPGNTPLLFSTYVMGRMDTYFEDPLTFN |
| | | PDRFGPGAPKPRFTYFPFSLGHRSCI |
| | NIPBL_0 | YQQTTISHSPSSRFVPPQTSSGNRFMPQQNSPVPSPY |
| | | APQSPAGYMPYSHPSSYTTHPQMQQ |
| | NIPBL_1 | SSRFVPPQTSSGNRFMPQQNSPVPSPYAPQSPAGYM |
| | | PYSHPSSYTTHPQMQQASVSSPIVAG |
| | FOXL2 | AHHLHAAAAPPPAPPHHGAAAPPPGQLSPASPATAA |
| | | PPAPAPTSAPGLQFACARQPELAMMH |
| | PLEKHG5 | AGTHGTPSAPSRSLSELCLAVPAPGIRTQGSPQEAGP |
| | | SWDCRGAPSPGSGPGLVGCLAGEPA |
| 2442 | COL4A2 | AGECRCTEGDEAIKGLPGLPGPKGFAGINGEPGRKG |
| | | DRGDPGQHGLPGFPGLKGVPGNIGAP |
| | COL11A1 | DDGMRGEDGEIGPRGLPGEAGPRGLLGPRGTPGAP |
| | | GQPGMAGVDGPPGPKGNMGPQGEPGPP |
| | PSD4 | SQDRDEREGGHPQESLPCTLAPCPWRSPASSPEPSSP |
| | | ESESRGPGPRPSPASSQEGSPQLQH |
| | MAP4K1_0 | ESSDDDYDDVDIPTPAEDTPPPLPPKPKFRSPSDEGP |
| | | GSMGDDGQLSPGVLVRCASGPPPNS |
| | MAP4K1_1 | PSDEGPGSMGDDGQLSPGVLVRCASGPPPNSPRPGP |
| | | PPSTSSPHLTAHSEPSLWNPPSRELD |
| 2443 | GDF5_0 | HSYGGGATNANARAKGGTGQTGGLTQPKKDEPKK |
| | | LPPRPGGPEPKPGHPPQTRQATARTVTP |
| 2444 | GDF5_1 | FHCEGLCEFPLRSHLEPTNHAVIQTLMNSMDPESTPP |
| | | TCCVPTRLSPISILFIDSANNVVYK |
| | COL3A1_0 | GPGAAGFPGARGLPGPPGSNGNPGPPGPSGSPGKDG |
| | | PPGPAGNTGAPGSPGVSGPKGDAGQP |
| | COL3A1_1 | AAGIKGHRGFPGNPGAPGSPGPAGQQGAIGSPGPAG |
| | | PRGPVGPSGPPGKDGTSGHPGPIGPP |
| 2445 | SMARCA2 | APEHVSSPMSGGGPTPPQMPPSQPGALIPGDPQAMS |
| | | QPNRGPSPFSPVQLHQLRAQILAYKM |
| | TBKBP1_0 | SSLQGRILRTLLQEQARSGGQRHSPLSQRHSPAPQCP |
| | | SPSPPARAAPPCPPCQSPVPQRRSP |
| | TBKBP1 | SPAPQCPSPSPPARAAPPCPPCQSPVPQRRSPVPPCPS |
| | | PQQRRSPASPSCPSPVPQRRSPVP |
| | TBKBP1_2 | RAAPPCPPCQSPVPQRRSPVPPCPSPQQRRSPASPSCP |
| | | SPVPQRRSPVPPSCQSPSPQRRSP |
| | TBKBP1_3 | CQSPVPQRRSPVPPCPSPQQRRSPASPSCPSPVPQRRS |
| | | PVPPSCQSPSPQRRSPVPPSCPAP |
| | INSYN1 | LDVSTPSDSVDGPESTRPGAGPDYRLMNGGTPIPNG |
| | | PRVETPDSSSEEAFGAGPTVKSQLPQ |
| 2446 | OAS3 | LRGMGDPVQSWKGPGLPRAGCSGLGHPIQLDPNQK |
| | | TPENSKSLNAVYPRAGSKPPSCPAPGP |
| | PLEKHA4 | HRMMTGGNLDSQGDPLPGVPLPPSDPTRQETPPPRS |
| | | PPVANSGSTGFSRRGSGRGGGPTPWG |
| 2447 | PLCG1 | GGWWRGDYGGKKQLWFPSNYVEEMVNPVALEPE |
| | | REHLDENSPLGDLLRGVLDVPACQIAIRP |
| | GIGYF2 | LSQIPSDTASPLLILPPPVPNPSPTLRPVETPVVGAPG |
| | | MGSVSTEPDDEEGLKHLEQQAEKM |
| | YIF1B | AVDTMYVGRKLGLLFFPYLHQDWEVQYQQDTPVA |
| | | PRFDVNAPDLYIPAMAFITYVLVAGLAL |
| | EIF4ENIF1 | SQANRYTKEQDYRPKATGRKTPTLASPVPTTPFLRP |
| | | VHQVPLVPHVPMVRPAHQLHPGLVQR |
| 2448 | ODAD1 | LADAALLVLGQSLEDLPKKMAPLQPPDTLEDPPGFE |
| | | ASDDYPMSREELLSQVEKLVELQEQA |
| | KAT5 | IPGGEPDQPLSSSSCLQPNHRSTKRKVEVVSPATPVP |
| | | SETAPASVFPQNGAARRAVAAQPGR |
| | MICALL1_0 | IMTYVSQYYNHFCSPGQAGVSPPRKGLAPCSPPSVA |
| | | PTPVEPEDVAQGEELSSGSLSEQGTG |
| | MICALL1_1 | PFEEEEEDKEEEAPAAPSLATSPALGHPESTPKSLHP |
| | | WYGITPTSSPKTKKRPAPRAPSASP |
| | MICALL1_2 | EAPAAPSLATSPALGHPESTPKSLHPWYGITPTSSPK |
| | | TKKRPAPRAPSASPLALHASRLSHS |
| | MICALL1_3 | APSLATSPALGHPESTPKSLHPWYGITPTSSPKTKKR |
| | | PAPRAPSASPLALHASRLSHSEPPS |
| | MED26 | HTSSPGLGKPPGPCLQPKASVLQQLDRVDETPGPPH |
| | | PKGPPRCSFSPRNSRHEGSFARQQSL |
| | ANKRD40_0 | VPNYLANPAFPFIYTPTAEDSAQMQNGGPSTPPASPP |
| | | ADGSPPLLPPGEPPLLGTFPRDHTS |
| | ANKRD40_1 | PFIYTPTAEDSAQMQNGGPSTPPASPPADGSPPLLPP |
| | | GEPPLLGTFPRDHTSLALVQNGDVS |
| 2449 | IL17RA_0 | QDAPSLDEEVFEEPLLPPGTGIVKRAPLVREPGSQAC |
| | | LAIDPLVGEEGGAAVAKLEPHLQPR |
| 2450 | IL17RA_1 | FEEPLLPPGTGIVKRAPLVREPGSQACLAIDPLVGEE |
| | | GGAAVAKLEPHLQPRGQPAPQPLHT |
| | DBP | AALPAATTPGPGLETAGPADAPAGAVVGGGSPRGR |
| | | PGPVPAPGLLAPLLWERTLPFGDVEYV |
| | FHIP1B_0 | ALFLRQQSLGGSESPGPAPCSPGLSASPASSPGRRPT |
| | | PAEEPGELEDNYLEYLREARRGVDR |
| | FHIP1B_1 | SPLEPPLPLEEEEAYESFTCPPEPPGPFLSSPLRTLNQL |
| | | PSQPFTGPFMAVLFAKLENMLQN |
| 2451 | CANX | FEILVDQSVVNSGNLLNDMTPPVNPSREIEDPEDRKP |
| | | EDWDERPKIPDPEAVKPDDWDEDAP |
| | EXOSC10 | ALADFIHQQRTQQVEQDMFAHPYQYELNHFTPADA |
| | | VLQKPQPQLYRPIEETPCHFISSLDEL |
| 2452 | STAT2 | SQTVPEPDQGPVSQPVPEPDLPCDLRHLNTEPMEIFR |
| | | NCVKIEEIMPNGDPLLAGQNTVDEV |
| 2453 | DBX2 | FGNLGKSFLIENLLRVGGAPTPRLQPPAPHDPATAL |
| | | ATAGAQLRPLPASPVPLKLCPAAEQV |
| | KRTAP10-7 | CSDSWQVDDCPESCCEPPCCAPAPCLSLVCTPVSYV |
| | | SSPCCRVTCEPSPCQSGCTSSCTPSC |
| 2454 | KIAA0754_0 | HAPEEPDTAAVRVSTPEEPASPAAAVPTPEEPTSPAA |
| | | AVPTPEEPTSPAAAVPPPEEPTSPA |
| 2455 | KIAA0754_1 | STPEEPASPAAAVPTPEEPTSPAAAVPTPEEPTSPAA |
| | | AVPPPEEPTSPAAAVPTPEEPTSPA |
| 2456 | KIAA0754_2 | PTPEEPTSPAAAVPTPEEPTSPAAAVPPPEEPTSPAAA |
| | | VPTPEEPTSPAAAVPTPEEPTSPA |
| 2457 | KIAA0754_3 | PTPEEPTSPAAAVPPPEEPTSPAAAVPTPEEPTSPAAA |
| | | VPTPEEPTSPAAAVPTPEEPTSPA |
| 2458 | KIAA0754_4 | PPPEEPTSPAAAVPTPEEPTSPAAAVPTPEEPTSPAAA |
| | | VPTPEEPTSPAAAVPTPEEPTSPA |
| 2459 | KIAA0754_5 | PTPEEPTSPAAAVPTPEEPTSPAAAVPTPEEPTSPAAA |
| | | VPTPEEPTSPAAAVPTPEEPTSPA |
| 2460 | KIAA0754_6 | PTPEEPTSPAAAVPTPEEPTSPAAAVPTPEEPTSPAAA |
| | | VPTPEEPTSPAAAVPTPEEPASPA |
| 2461 | KIAA0754_7 | PTPEEPTSPAAAVPTPEEPTSPAAAVPTPEEPTSPAAA |
| | | VPTPEEPASPAAAVPTPEEPASPA |
| 2462 | KIAA0754_8 | PTPEEPTSPAAAVPTPEEPTSPAAAVPTPEEPASPAA |
| | | AVPTPEEPASPAAAVPTPEEPAFPA |
| 2463 | KIAA0754_9 | PTPEEPTSPAAAVPTPEEPASPAAAVPTPEEPASPAA |
| | | AVPTPEEPAFPAPAVPTPEESASAA |
| | KIAA0754_10 | EEPTSPAAAVPTPEEPASPAAAVPTPEEPASPAAAVP |
| | | TPEEPAFPAPAVPTPEESASAAVAV |
| 2464 | KIAA0754_11 | PTPEEPASPAAAVPTPEEPASPAAAVPTPEEPAFPAP |
| | | AVPTPEESASAAVAVPTPEESASPA |
| | KIAA0754_12 | AAVPTPEEPASPAAAVPTPEEPAFPAPAVPTPEESAS |
| | | AAVAVPTPEESASPAAAVPTPAESA |
| | KIAA0754_13 | AVVATLEEPTSPAASVPTPAAMVATLEEFTSPAASV |
| | | PTSEEPASLAAAVSNPEEPTSPAAAV |
| 2465 | KIAA0754_14 | SPAASVPTPAAMVATLEEFTSPAASVPTSEEPASLAA |
| | | AVSNPEEPTSPAAAVPTLEEPTSSA |
| 2466 | KIAA0754_15 | PTSEEPASLAAAVSNPEEPTSPAAAVPTLEEPTSSAA |
| | | AVLTPEELSSPAASVPTPEEPASPA |
| | ATG9B_0 | FSPPTAGPPCSVLQGTGASQSCHSALPIPATPPTQAQ |
| | | PAMTPASASPSWGSHSTPPLAPATP |
| | ATG9B_1 | SVLQGTGASQSCHSALPIPATPPTQAQPAMTPASASP |
| | | SWGSHSTPPLAPATPTPSQQCPQDS |
| | ATG9B_2 | TGASQSCHSALPIPATPPTQAQPAMTPASASPSWGS |
| | | HSTPPLAPATPTPSQQCPQDSPGLRV |
| | ATG9B_3 | PTQAQPAMTPASASPSWGSHSTPPLAPATPTPSQQC |
| | | PQDSPGLRVGPLIPEQDYERLEDCDP |
| | ILF3 | RDSSKGEDSAEETEAKPAVVAPAPVVEAVSTPSAAF |
| | | PSDATAEQGPILTKHGKNPVMELNEK |
| | SLC25A46 | RSFSTGSDLGHWVTTPPDIPGSRNLHWGEKSPPYGV |
| | | PTTSTPYEGPTEEPFSSGGGGSVQGQ |
| | CBS | PEDKEAKEPLWIRPDAPSRCTWQLGRPASESPHHHT |
| | | APAKSPKILPDILKKIGDTPMVRINK |
| | PELP1 | SSFCSEALVTCAALTHPRVPPLQPMGPTCPTPAPVPP |
| | | PEAPSPFRAPPFHPPGPMPSVGSMP |
| | PAK5 | QKFTGLPQQWHSLLADTANRPKPMVDPSCITPIQLA |
| | | PMKTIVRGNKPCKETSINGLLEDFDN |
| 2467 | VHL | PEESGPEELGAEEEMEAGRPRPVLRSVNSREPSQVIF |
| | | CNRSPRVVLPVWLNFDGEPQPYPTL |
| | NR4A3_0 | DPPMKAVPTVAGARFPLFHFKPSPPHPPAPSPAGGH |
| | | HLGYDPTAAAALSLPLGAAAAAGSQA |
| | NR4A3_1 | GSQAAALESHPYGLPLAKRAAPLAFPPLGLTPSPTAS |
| | | SLLGESPSLPSPPSRSSSSGEGTCA |
| 2468 | TRIM11 | PNRPLAKMAEMARRLHPPSPVPQGVCPAHREPLAA |
| | | FCGDELRLLCAACERSGEHWAHRVRPL |
| | TFAP2A_0 | HDGTSNGTARLPQLGTVGQSPYTSAPPLSHTPNADF |
| | | QPPYFPPPYQPIYPQSQDPYSHVNDP |
| 2469 | TFAP2A_1 | TPNADFQPPYFPPPYQPIYPQSQDPYSHVNDPYSLNP |
| | | LHAQPQPQHPGWPGQRQSQESGLLH |
| | FAM161A | IKREKILADIEADEENLKETRWPYLSPRRKSPVRCAG |
| | | VNPVPCNCNPPVPTVSSRGREQAVR |
| | ADAMTS14_0 | HRLCCVSCIKKASGPNPGPDPGPTSLPPFSTPGSPLPG |
| | | PQDPADAAEPPGKPTGSEDHQHGR |
| 2470 | ADAMTS14_1 | KASGPNPGPDPGPTSLPPFSTPGSPLPGPQDPADAAE |
| | | PPGKPTGSEDHQHGRATQLPGALDT |
| | FNDC3A | VQVNPGEAFTIRREDGQFQCITGPAQVPMMSPNGSV |
| | | PPIYVPPGYAPQVIEDNGVRRVVVVP |
| 2471 | PARL | PQLLGRRFNFFIQQKCGFRKAPRKVEPRRSDPGTSG |
| | | EAYKRSALIPPVEETVFYPSPYPIRS |
| 2472 | GDF6_0 | RSRKEGKMQRAPRDSDAGREGQEPQPRPQDEPRAQ |
| | | QPRAQEPPGRGPRVVPHEYMLSIYRTY |
| 2473 | GDF6_1 | APRDSDAGREGQEPQPRPQDEPRAQQPRAQEPPGR |
| | | GPRVVPHEYMLSIYRTYSIAEKLGINA |
| | GDF6_2 | GAELRLFRQAPSAPWGPPAGPLHVQLFPCLSPLLLD |
| | | ARTLDPQGAPPAGWEVFDVWQGLRHQ |
| 2474 | GDF6_3 | YHCEGVCDFPLRSHLEPTNHAIIQTLMNSMDPGSTP |
| | | PSCCVPTKLTPISILYIDAGNNVVYK |
| 2475 | ACHE | LVTVRGGRLRGIRLKTPGGPVSAFLGIPFAEPPMGPR |
| | | RFLPPEPKQPWSGVVDATTFQSVCY |
| | ZMYND8 | SASEESMDFLDKSTASPASTKTGQAGSLSGSPKPFSP |
| | | QLSAPITTKTDKTSTTGSILNLNLD |
| | SOX8 | QGDYGDLQASSYYGAYPGYAPGLYQYPCFHSPRRP |
| | | YASPLLNGLALPPAHSPTSHWDQPVYT |
| | ROBO4 | QTQPPVAPQAPSSILLPAAPIPILSPCSPPSPQASSLSG |
| | | PSPASSRLSSSSLSSLGEDQDSV |
| | MYO15A_0 | SPPVPPRPPSSGPPPAPPLSPALSGLPRPASPYGSLRR |
| | | HPPPWAAPAHVPPAPQASWWAFVE |
| 2476 | MYO15A_1 | PYGSLRRHPPPWAAPAHVPPAPQASWWAFVEPPAV |
| | | SPEVPPDLLAFPGPRPSFRGSRRRGAA |
| | MYO15A_2 | RRHPPPWAAPAHVPPAPQASWWAFVEPPAVSPEVP |
| | | PDLLAFPGPRPSFRGSRRRGAAFGFPG |
| | MYO15A_3 | PPFLPPARRPRSLQESPAPRRAAGRLGPPGSPLPGSPR |
| | | PPSPPLGLCHSPRRSSLNLPSRLP |
| | MYO15A_4 | SLPAEKPPAPEAQPTSVGTGPPAKPVLLRATPKPLAP |
| | | APLAKAPRLPIKPVAAPVLAQDQAS |
| 2477 | NCOR2_0 | NGPKPPATLGADGPPPGPPTPPPEDIPAPTEPTPASEA |
| | | TGAPTPPPAPPSPSAPPPVVPKEE |
| | NCOR2_1 | PKPPATLGADGPPPGPPTPPPEDIPAPTEPTPASEATG |
| | | APTPPPAPPSPSAPPPVVPKEEKE |
| | NCOR2_2 | GPPPGPPTPPPEDIPAPTEPTPASEATGAPTPPPAPPSP |
| | | SAPPPVVPKEEKEEETAAAPPVE |
| | ELK3 | AAAASAFLASSVSAKISSLMLPNAASISSASPFSSRSP |
| | | SLSPNSPLPSEHRSLFLEAACHDS |
| 2478 | SIRT2 | PSTGLYDNLEKYHLPYPEAIFEISYFKKHPEPFFALA |
| | | KELYPGQFKPTICHYFMRLLKDKGL |
| | E2F7_0 | VGPSSGQLPSFSVPCMVLPSPPLGPFPVLYSPAMPGP |
| | | VSSTLGALPNTGPVNFSLPGLGSIA |
| | E2F7_1 | SHSVVQQPESPVYVGHPVSVVKLHQSPVPVTPKSIQ |
| | | RTHRETFFKTPGSLGDPVLKRRERNQ |
| 2479 | CDHR5 | QAFLPDHKANWAPVPSPTHDPKPAEAPMPAEPAPP |
| | | GPASPGGAPEPPAAARAGGSPTAVRSI |
| 2480 | KLF4_0 | PPPTAPFNLADINDVSPSGGFVAELLRPELDPVYIPPQ |
| | | QPQPPGGGLMGKFVLKASLSAPGS |
| | KLF4_1 | GLMGKFVLKASLSAPGSEYGSPSVISVSKGSPDGSH |
| | | PVVVAPYNGGPPRTCPKIKQEAVSSC |
| | PKD1 | WEPLKVLLEALYFSLVAKRLHPDEDDTLVESPAVTP |
| | | VSARVPRVRPPHGFALFLAKEEARKV |
| | ATXN2_0 | VPWPSPCPSPSSRPPSRYQSGPNSLPPRAATPTRPPSR |
| | | PPSRPSRPPSHPSAHGSPAPVSTM |
| | ATXN2_1 | NPNAKEFNPRSFSQPKPSTTPTSPRPQAQPSPSMVGH |
| | | QQPTPVYTQPVCFAPNMMYPVPVSP |
| | ATXN2_2 | SFSQPKPSTTPTSPRPQAQPSPSMVGHQQPTPVYTQP |
| | | VCFAPNMMYPVPVSPGVQPLYPIPM |
| | ATXN2_3 | SPSMVGHQQPTPVYTQPVCFAPNMMYPVPVSPGVQ |
| | | PLYPIPMTPMPVNQAKTYRAVPNMPQQ |
| | KNG1 | IQSDDDWIPDIQIDPNGLSFNPISDFPDTTSPKCPGRP |
| | | WKSVSEINPTTQMKESYYFDLTDG |
| 2481 | TUBGCP6 | SDVVSTRPRWNTHVPIPPPHMVLGALSPEAEPNTPR |
| | | PQQSPPGHTSQSALSLGAQSTVLDCG |
| | ULK1 | SHGLQSCRNLRGSPKLPDFLQRNPLPPILGSPTKAVP |
| | | SFDFPKTPSSQNLLALLARQGVVMT |
| 2482 | WEE1_0 | FSPCSDCEEEEEEEEEEGSGHSTGEDSAFQEPDSPLPP |
| | | ARSPTEPGPERRRSPGPAPGSPGE |
| | WEE1_1 | EEEEEEEGSGHSTGEDSAFQEPDSPLPPARSPTEPGPE |
| | | RRRSPGPAPGSPGELEEDLLLPGA |
| 2483 | WEE1_2 | EEEEGSGHSTGEDSAFQEPDSPLPPARSPTEPGPERR |
| | | RSPGPAPGSPGELEEDLLLPGACPG |
| | WEE1_3 | FQEPDSPLPPARSPTEPGPERRRSPGPAPGSPGELEED |
| | | LLLPGACPGADEAGGGAEGDSWEE |
| | COL2A1_0 | PAGEQGPRGDRGDKGEKGAPGPRGRDGEPGTPGNP |
| | | GPPGPPGPPGPPGLGGNFAAQMAGGFD |
| 2484 | COL2A1_1 | PMGPMGPRGPPGPAGAPGPQGFQGNPGEPGEPGVS |
| | | GPMGPRGPPGPPGKPGDDGEAGKPGKA |
| 2485 | COL2A1_2 | EQGPKGEPGPAGPQGAPGPAGEEGKRGARGEPGGV |
| | | GPIGPPGERGAPGNRGFPGQDGLAGPK |
| | COL2A1_3 | LVGPRGERGFPGERGSPGAQGLQGPRGLPGTPGTDG |
| | | PKGASGPAGPPGAQGPPGLQGMPGER |
| 2486 | COL2A1_4 | PKGARGDSGPPGRAGEPGLQGPAGPPGEKGEPGDD |
| | | GPSGAEGPPGPQGLAGQRGIVGLPGQR |
| | COL2A1_5 | APGASGDRGPPGPVGPPGLTGPAGEPGREGSPGADG |
| | | PPGRDGAAGVKGDRGETGAVGAPGAP |
| 2487 | AMH | APLPAHGQLDTVPFPPPRPSAELEESPPSADPFLETLT |
| | | RLVRALRVPPARASAPRLALDPDA |
| | CACNA1G | LQLPKDAPHLLQPHSAPTWGTIPKLPPPGRSPLAQRP |
| | | LRRQAAIRTDSLDVQGLGSREDLLA |
| | PTK2_0 | RMESRRQATVSWDSGGSDEAPPKPSRPGYPSPRSSE |
| | | GFYPSPQHMVQTNHYQVSGYPGSHGI |
| | PTK2_1 | SWDSGGSDEAPPKPSRPGYPSPRSSEGFYPSPQHMV |
| | | QTNHYQVSGYPGSHGITAMAGSIYPG |
| | TAB3 | QSSPQGPVPHYSQRPLPVYPHQQNYQPSQYSPKQQQ |
| | | IPQSAYHSPPPSQCPSPFSSPQHQVQ |
| 2488 | FCRLA_0 | GPGIPETASVVAITVQELFPAPILRAVPSAEPQAGSP |
| | | MTLSCQTKLPLQRSAARLLFSFYKD |
| | FCRLA_1 | ETASVVAITVQELFPAPILRAVPSAEPQAGSPMTLSC |
| | | QTKLPLQRSAARLLFSFYKDGRIVQ |
| | PTCH1 | LNGLVLLPVLLSFFGPYPEVSPANGLNRLPTPSPEPPP |
| | | SVVRFAMPPGHTHSGSDSSDSEYS |
| 2489 | REST | KKQNTCMKKSTKKKTLKNKSSKKSSKPPQKEPVEK |
| | | GSAQMDPPQMGPAPTEAVQKGPVQVEP |
| | ZNF804A | CEVYQHILQPNMLANKVKFTFPPAALPPPSTPLQPLP |
| | | LQQSLCSTSVTTIHHTVLQQHAAAA |
| | RGS12 | VQESSDSPSTSPGSASSPPGPPGTTPPGQKSPSGPFCT |
| | | PQSPVSLAQEGTAQIWKRQSQEVE |
| 2490 | COL5A1_0 | PSEIGPGMPANQDTIYEGIGGPRGEKGQKGEPAIIEP |
| | | GMLIEGPPGPEGPAGLPGPPGTMGP |
| 2491 | COL5A1_1 | PGMPANQDTIYEGIGGPRGEKGQKGEPAIIEPGMLIE |
| | | GPPGPEGPAGLPGPPGTMGPTGQVG |
| 2492 | COL5A1_2 | RLALRGPAGPMGLTGRPGPVGPPGSGGLKGEPGDV |
| | | GPQGPRGVQGPPGPAGKPGRRGRAGSD |
| 2493 | COL5A1_3 | IKGDRGEIGPPGPRGEDGPEGPKGRGGPNGDPGPLG |
| | | PPGEKGKLGVPGLPGYPGRQGPKGSI |
| | COL5A1_4 | FPGDRGLPGPVGALGLKGNEGPPGPPGPAGSPGERG |
| | | PAGAAGPIGIPGRPGPQGPPGPAGEK |
| | COL5A1_5 | ERGEKGESGPSGAAGPPGPKGPPGDDGPKGSPGPVG |
| | | FPGDPGPPGEPGPAGQDGPPGDKGDD |
| | COL5A1_6 | PIGPQGAPGKPGPDGLRGIPGPVGEQGLPGSPGPDGP |
| | | PGPMGPPGLPGLKGDSGPKGEKGHP |
| | PAK4_0 | APNGPSAGGLAIPQSSSSSSRPPTRARGAPSPGVLGP |
| | | HASEPQLAPPACTPAAPAVPGPPGP |
| 2494 | PAK4_1 | AIPQSSSSSSRPPTRARGAPSPGVLGPHASEPQLAPPA |
| | | CTPAAPAVPGPPGPRSPQREPQRV |
| 2495 | CAMSAP2 | YLVFMAELFWWFEVVKPSFVQPRVVRPQGAEPVK |
| | | DMPSIPVLNAAKRNVLDSSSDFPSSGEG |
| 2496 | FGF21 | ELLLEDGYNVYQSEAHGLPLHLPGNKSPHRDPAPR |
| | | GPARFLPLPGLPPALPEPPGILAPQPP |
| | SFPQ | GVGSAPPASSSAPPATPPTSGAPPGSGPGPTPTPPPAV |
| | | TSAPPGAPPPTPPSSGVPTTPPQA |
| | ANKRD11_0 | DSPMPPSMEDRAPLPPVPAEKFACLSPGYYSPDYGL |
| | | PSPKVDALHCPPAAVVTVTPSPEGVF |
| 2497 | ANKRD11_1 | DGAGPEDDTEASRAAAPAEGPPGGIQPEAAEPKPTA |
| | | EAPKAPRVEEIPQRMTRNRAQMLANQ |
| | TICRR_0 | TPRTPKRQGTQPPGFLPNCTWPHSVNSSPESPSCPAP |
| | | PTSSTAQPRRECLTPIRDPLRTPPR |
| | TICRR_1 | PALSMPRASRSLSKPEPTYVSPPCPRLSHSTPGKSRG |
| | | QTYICQACTPTHGPSSTPSPFQTDG |
| | PSMB8 | APRGQRPESALPVAGSGRRSDPGHYSFSMRSPELAL |
| | | PRGMQPTEFFQSLGGDGERNVQIEMA |
| 2498 | CARMIL2_0 | LSAARDQLVESLAQQATVTMPPALPAPDGGEPSLLE |
| | | PGELEGLFFPEEKEEEKEKDDSPPQK |
| 2499 | CARMIL2_1 | DQLVESLAQQATVTMPPALPAPDGGEPSLLEPGELE |
| | | GLFFPEEKEEEKEKDDSPPQKWPELS |
| 2500 | ESRRA | SSQVVGIEPLYIKAEPASPDSPKGSSETETEPPVALAP |
| | | GPAPTRCLPGHKEEEDGEGAGPGE |
| | STIM1_0 | LAKKALLALNHGLDKAHSLMELSPSAPPGGSPHLDS |
| | | SRSHSPSSPDPDTPSPVGDSRALQAS |
| | STIM1_1 | HGLDKAHSLMELSPSAPPGGSPHLDSSRSHSPSSPDP |
| | | DTPSPVGDSRALQASRNTRIPHLAG |
| 2501 | STIM1_2 | AHSLMELSPSAPPGGSPHLDSSRSHSPSSPDPDTPSPV |
| | | GDSRALQASRNTRIPHLAGKKAVA |
| | CAPN15 | MLEPGEYAVVCCAFNHWGPPLPGTPAPQASSPSAG |
| | | VPRASPEPPGHVLAVYSSRLVMVEPVE |
| 2502 | GRID2IP | SHPYASLDSSRAPSPQPGPGPICPDSPPSPDPTRPPSR |
| | | RKLFTFSHPVRSRDTDRFLDVLSE |
| | BAHCC1 | PTAPGAPSPAAGPTKLPPCCHPPDPKPPASSPTPPPRP |
| | | SAPCTLNVCPASSPGPGSRVRSAE |
| 2503 | MAGI1 | QQQQQQTEEWTEDHSALVPPVIPNHPPSNPEPAREV |
| | | PLQGKPFFTRNPSELKGKFIHTKLRK |
| 2504 | ZBTB20 | FDSGVSSSIGTEPDSVEQQFGPGAARDSQAEPTQPEQ |
| | | AAEAPAEGGPQTNQLETGASSPERS |
| | KIAA1210_0 | QVIIRGLPVWFSHFQGILEGSLQCVTQTLETPNLDEP |
| | | LPVEPKEEEPNLPLVSEEEKSITKP |
| 2506 | KIAA1210_1 | GLPVWFSHFQGILEGSLQCVTQTLETPNLDEPLPVEP |
| | | KEEEPNLPLVSEEEKSITKPKEINE |
| 2507 | KIAA1210_2 | FSHFQGILEGSLQCVTQTLETPNLDEPLPVEPKEEEP |
| | | NLPLVSEEEKSITKPKEINEKKLGM |
| 2508 | KIAA1210_3 | GILEGSLQCVTQTLETPNLDEPLPVEPKEEEPNLPLV |
| | | SEEEKSITKPKEINEKKLGMDSADS |
| | KIAA1210_4 | GNLTKISYVADKQQSRPKSESMAKKQPACKTPGKP |
| | | AGQQSDYAVSEPVWITMAKQKQKSFKA |
| | MYO9B_0 | ASTESLLEERAGRGASEGPPAPALPCPGAPTPSPLPT |
| | | VAAPPRRRPSSFVTVRVKTPRRTPI |
| | MYO9B_1 | WAPGAREAAAPVRRREPPARRPDQIHSVYITPGADL |
| | | PVQGALEPLEEDGQPPGAKRRYSDPP |
| | TRPM2_0 | FRGAVYHSYLTIFGQIPGYIDGVNFNPEHCSPNGTDP |
| | | YKPKCPESDATQQRPAFPEWLTVLL |
| 2509 | TRPM2_1 | YHSYLTIFGQIPGYIDGVNFNPEHCSPNGTDPYKPKC |
| | | PESDATQQRPAFPEWLTVLLLCLYL |
| 2510 | TRPM2_2 | ARHLLYPNCPVTRFPVPNEKVPWETEFLIYDPPFYT |
| | | AERKDAAAMDPMGDTLEPLSTIQYNV |
| | TBX10 | AFLSAGLGILAPSETYPLPTTSSGWEPRLGSPFPSGPC |
| | | TSSTGAQAVAEPTGQGPKNPRVSR |
| | C11orf53 | ALLEPYFPQEPYGDYRPPALTPNAGSLFSASPLPPLL |
| | | PPPFPGDPAHFLFRDSWEQTLPDGL |
| 2511 | GNL1 | QIQEPYTAVGYLASRIPVQALLHLRHPEAEDPSAEHP |
| | | WCAWDICEAWAEKRGYKTAKAARND |
| | UNC13A | LPPAAPGKEDKAPVAPTEAPDMAKVAPKPATPDKV |
| | | PAAEQIPEAEPPKDEESFRPREDEEGQ |
| | AGAP2_0 | VPPGPPLSGGLSPDPKPGGAPTSSRRPLLSSPSWGGP |
| | | EPEGRAGGGIPGSSSPHPGTGSRRL |
| | AGAP2_1 | KGKSKTLDNSDLHPGPPAGSPPPLTLPPTPSPATAVT |
| | | AASAQPPGPAPPITLEPPAPGLKRG |
| 2512 | ZNF517 | HHRLHAQEGAQDGGVGQGALLGAAQRPQAGDPPH |
| | | ECPVCGRPFRHNSLLLLHLRLHTGEKPF |
| | SOCS1 | VAHNQVAADNAVSTAAEPRRRPEPSSSSSSSPAAPA |
| | | RPRPCPAVPAPAPGDTHFRTFRSHAD |
| | SPATA31D4 | LADLFSPSPLRDPLPPQPVSPLDSKFPIDHSPPQQLPF |
| | | PLLPPHHIERVEPSLQPEASLSLN |
| 2513 | KIAA1671_0 | PFSKEQDVKSPVPSLRPSSTGPSPSGGLSEEPAAKDL |
| | | DNRMPGLVGQEVGSGEGPRTSSPLF |
| | KIAA1671_1 | IIDVDALWSHRGSEDGPRPQSNWKESANKMSPSGG |
| | | APQTTPTLRSRPKDLPVRRKTDVISDT |
| 2514 | ERFL | PSPFGGAPGPDAPPLTPETLQTLFSAPRLGEPGARTP |
| | | LFTSETDKLRLDSPFPFLGSGATSY |
| | PROX2 | RVQLQAGVPVGNLSLAKRLDSPRYPIPPRMTPKPCQ |
| | | DPPANFPLTAPSHIQENQILSQLLGH |
| | LRRC37A3 | PEHSHLTQATVQPLDLGFTITPESMTEVELSPTMKET |
| | | PTQPPKKVVPQLRVYQGVTNPTPGQ |
| 2515 | MROH1_0 | AMAHHGYLEQPGGEAMIEYIVQQCALPPEQEPEKP |
| | | GPGSKDPKADSVRAISVRTLYLVSTTV |
| 2516 | MROH1_1 | PGGEAMIEYIVQQCALPPEQEPEKPGPGSKDPKADS |
| | | VRAISVRTLYLVSTTVDRMSHVLWPY |
| | POM121L2 | TIWSLRHPRPIWSPVTIRITPPDQRVPPSTSPEDVIALA |
| | | GLPPSEELADPCSKETVLRALRE |
| 2517 | MIER2 | LPSSEPGPCSFQQLDESPAVPLSHRPPALADPASYQP |
| | | AVTAPEPDASPRLAVDFALPKELPL |
| | LRRC66 | SAHYSEVPYGDPRDTGPSVFPPRWDSGLDVTPANK |
| | | EPVQKSTPSDTCCELESDCDSDEGSLF |
| | KIF26A_0 | LQAPASHEDLDAPHGGPSLAPPSTTTSSRDTPGPAGP |
| | | AGRQPGRAGPDRTKGLAWSPGPSVQ |
| | KIF26A_1 | TSSRDTPGPAGPAGRQPGRAGPDRTKGLAWSPGPS |
| | | VQVSVAPAGLGGALSTVTIQAQQCLEG |
| 2518 | KIF26A_2 | RIWPAQGAQRSAEAMSFLKVDPRKKQVILYDPAAG |
| | | PPGSAGPRRAATAAVPKMFAFDAVFPQ |
| 2519 | KIF26A_3 | SSSGGESSCEEGRARRPPHLRPFHPRTVALDPDRTPP |
| | | CLPGDPDYSSSSEQSCDTVIYVGPG |
| | KIF26A_4 | TFAELQERLECMDGNEGPSGGPGGTDGAQASPARG |
| | | GRKPSPPEAASPRKAVGTPMAASTPRG |
| | KIF26A_5 | LAPKAGFLPRPSGAAPPAPPTRKSSLEQRSSPASAPP |
| | | HAVNPARVGAAAVLRGEEEPRPSSR |
| 2520 | PRRC2B_0 | SLKSENKGNDPNIVIVPKDGTGWANKQDQQDPKSS |
| | | SATASQPPESLPQPGLQKSVSNLQKPT |
| | PRRC2B_1 | DQKCKQARKAGEARKQAEKEVPWSPSAEKASPQE |
| | | NGPAVHKGSPEFPAQETPTTFPEEAPTV |
| 2521 | DDR1 | NSSPALGGTFPPAPWWPPGPPPTNFSSLELEPRGQQP |
| | | VAKAEGSPTAILIGCLVAIILLLLL |
| | BNC1 | KGQPAFPNIGQNGVLFPNLKTVQPVLPFYRSPATPA |
| | | EVANTPGILPSLPLLSSSIPEQLISN |
| | SPATA31D3 | LADLFSPSPLRDPLPPQPVSPLDSKFPIDHSPPQQLPF |
| | | PLLPPHHIERVEPSLQPEASLSLN |
| | CXorf49_0 | ADTSRQASFHCKESYLPVPGRFLTSAPRGLTPVAER |
| | | PAVGELEDSPQKKMQSRAWGKVEVRP |
| 2522 | CXorf49_1 | RPGLPRLSVRRGEFSSSDPNIRAPQLPGTSEPSAYSPG |
| | | GLVPRRHAPSGNQQPPVHPPRPER |
| | CXorf49_2 | RLSVRRGEFSSSDPNIRAPQLPGTSEPSAYSPGGLVP |
| | | RRHAPSGNQQPPVHPPRPERQQQPP |
| | CXorf49B_0 | ADTSRQASFHCKESYLPVPGRFLTSAPRGLTPVAER |
| | | PAVGELEDSPQKKMQSRAWGKVEVRP |
| 2523 | CXorf49B_1 | RPGLPRLSVRRGEFSSSDPNIRAPQLPGTSEPSAYSPG |
| | | GLVPRRHAPSGNQQPPVHPPRPER |
| | CXorf49B_2 | RLSVRRGEFSSSDPNIRAPQLPGTSEPSAYSPGGLVP |
| | | RRHAPSGNQQPPVHPPRPERQQQPP |
| 2524 | PITPNM2 | IPALDVFQLRPACQQVYNLFHPADPSASRLEPLLERR |
| | | FHALPPFSVPRYQRYPLGDGCSTLL |
| 2525 | AHRR | ETPGPTKPLPWTAGKHSEDGARPRLQPSKNDPPSLR |
| | | PMPRGSCLPCPCVQGTFRNSPISHPP |
| | TNRC18_0 | ALKAKVIQKLEDVSKPPAYAYPATPSSHPTSPPPASP |
| | | PPTPGITRKEEAPENVVEKKDLELE |
| | TNRC18_1 | AATLEEGNPTDEVPSTPLALEPSSTPGSKKSPPEPVD |
| | | KRAKAPKARPAPPQPSPAPPAFTSC |
| 2526 | TNRC18_2 | VDKRAKAPKARPAPPQPSPAPPAFTSCPAPEPFAELP |
| | | APATSLAPAPLITMPATRPKPKKAR |
| 2527 | ODF3B | PHRPRGPIAAHYGGPGPKYKLPPNTGYALHDPSRPR |
| | | APAFTFGARFPTQQTTCGPGPGHLVP |
| 2528 | IL16 | PAASEARDPGVSESPPPGRQPNQKTLPPGPDPLLRLL |
| | | STQAEESQGPVLKMPSQRARSFPLT |
| 2529 | SRMS | LRRRLAFLSFFWDKIWPAGGEPDHGTPGSLDPNTDP |
| | | VPTLPAEPCSPFPQLFLALYDFTARC |
| | RNF225 | RPQLVALAPAPGFSWFPPRPPPGSPWAPAWTPRPTG |
| | | PDLDTALPGTAEDALEPEAGPEDPAE |
| 2530 | PCNX3_0 | RPPGPGLLSSEGPSGKWSLGGRKGLGGSDGEPASGS |
| | | PKGGTPKSQAPLDLSLSLSLSLSPDV |
| | PCNX3_1 | GLLSSEGPSGKWSLGGRKGLGGSDGEPASGSPKGG |
| | | TPKSQAPLDLSLSLSLSLSPDVSTEAS |
| | PCNX3_2 | EGPSGKWSLGGRKGLGGSDGEPASGSPKGGTPKSQ |
| | | APLDLSLSLSLSLSPDVSTEASPPRAS |
| | RGL4 | PRPGQHALTMPALEPAPPLLADLGPALEPESPAALG |
| | | PPGYLHSAPGPAPAPGEGPPPGTVLE |
| | SALL3_0 | PVEKEAEPMDAEPAGDTRAPRPPPAAPAPPTPAYGA |
| | | PSTNVTLEALLSTKVAVAQFSQGARA |
| | SALL3_1 | VPTSVGLQLPPTVPGAHGYADSPSATPASRSPQRPSP |
| | | ASSECASLSPGLNHVESGVSATAES |
| | SALL3_2 | GLQLPPTVPGAHGYADSPSATPASRSPQRPSPASSEC |
| | | ASLSPGLNHVESGVSATAESPQSLL |
| | SREBF1_0 | LQLINNQDSDFPGLFDPPYAGSGAGGTDPASPDTSSP |
| | | GSLSPPPATLSSSLEAFLSGPQAAP |
| | SREBF1_1 | SPGSLSPPPATLSSSLEAFLSGPQAAPSPLSPPQPAPTP |
| | | LKMYPSMPAFSPGPGIKEESVPL |
| | SHISA7 | DINVPRALVDILRHQAGPGTRPDRARSSSLTPGIGGP |
| | | DSMPPRTPKNLYNTVKTPNLDWRAL |
| | KIF24 | LPVSSATRHLWLSSSPPDNKPGGDLPALSPSPIRQHP |
| | | ADKLPSREADLGEACQSRETVLFSH |
| | C4orf54 | PETGQYVDVPMTSQQQAVAPMSISVPPLALSPGAY |
| | | GPTYMIYPGFLPTVLPTNALQPTPIAR |
| | NPIPB8 | PPSVDDNLKECLFVPLPPSPLPPSVDDNLKTPPLATQ |
| | | EAEVEKPPKPKRWRVDEVEQSPKPK |
| 2531 | ASCL5_0 | ALVDRRPLGPPSCMQLGVMPPPRQAPLPPAEPLGNV |
| | | PFLLYPGPAEPPYYDAYAGVFPYVPF |
| 2532 | ASCL5_1 | LGVMPPPRQAPLPPAEPLGNVPFLLYPGPAEPPYYD |
| | | AYAGVFPYVPFPGAFGVYEYPFEPAF |
| | ATXN2L | LKPQPLQQPSQPQQPPPTQQAVARRPPGGTSPPNGG |
| | | LPGPLATSAAPPGPPAAASPCLGPVA |
| | HDAC5 | SKEPTPGGLNHSLPQHPKCWGAHHASLDQSSPPQSG |
| | | PPGTPPSYKLPLPGPYDSRDDFPLRK |
| | ATF7IP_0 | WKETPCILSVNVKNKQDDDLNCEPLSPHNITPEPVS |
| | | KLPAEPVSGDPAPGDLDAGDPASGVL |
| 2533 | ATF7IP_1 | ETPCILSVNVKNKQDDDLNCEPLSPHNITPEPVSKLP |
| | | AEPVSGDPAPGDLDAGDPASGVLAS |
| 2534 | ATF7IP_2 | NVKNKQDDDLNCEPLSPHNITPEPVSKLPAEPVSGD |
| | | PAPGDLDAGDPASGVLASGDSTSGDP |
| 2535 | ATF7IP_3 | QDDDLNCEPLSPHNITPEPVSKLPAEPVSGDPAPGDL |
| | | DAGDPASGVLASGDSTSGDPTSSEP |
| 2536 | ATF7IP_4 | SPHNITPEPVSKLPAEPVSGDPAPGDLDAGDPASGVL |
| | | ASGDSTSGDPTSSEPSSSDAASGDA |
| 2537 | ATF7IP_5 | DATSGDAPSGDVSPGDATSGDATADDLSSGDPTSSD |
| | | PIPGEPVPVEPISGDCAADDIASSEI |
| 2538 | ATF7IP_6 | DAPSGDVSPGDATSGDATADDLSSGDPTSSDPIPGEP |
| | | VPVEPISGDCAADDIASSEITSVDL |
| 2539 | ATF7IP_7 | DVSPGDATSGDATADDLSSGDPTSSDPIPGEPVPVEP |
| | | ISGDCAADDIASSEITSVDLASGAP |
| 2540 | ATF7IP_8 | DATSGDATADDLSSGDPTSSDPIPGEPVPVEPISGDC |
| | | AADDIASSEITSVDLASGAPASTDP |
| 2541 | ATF7IP_9 | TTTYVVNNGLTLGSTGPQLTVHHRPPQVHTEPPRPV |
| | | HPAPLPEAPQPQRLPPEAASTSLPQK |
| | RNF217 | APASEQLSPPASPPGAPPVLNPPSTRSSFPSPRLSLPT |
| | | DSLSPDGGSIELEFYLAPEPFSMP |
| | ZNF831 | REAPWDSAPMASPGLPAASTQPWRKLPEQKSPTAG |
| | | KPCALQRQQATAAEKPWDAKAPEGRLR |
| 2542 | ITPKB | QPPEALVERQGQFLGSETSPAPERGGPRDGEPPGKM |
| | | GKGYLPCGMPGSGEPEVGKRPEETTV |
| 2543 | MAGE-like | MNNSVACSAFTVWCSHHRCLLPNRFIPPRGDPMCII |
| | | PPRGDPMCIIPPRGDPMWIITPRGDP |
| 2544 | MAGE-like | TVWCSHHRCLLPNRFIPPRGDPMCIIPPRGDPMCIIPP |
| | | RGDPMWIITPRGDPMCIIPPRGDP |
| 2545 | MAGE-like | LPNRFIPPRGDPMCIIPPRGDPMCIIPPRGDPMWIITPR |
| | | GDPMCIIPPRGDPMWIIPPRGDP |
| 2546 | MAGE-like | DPMCIIPPRGDPMCIIPPRGDPMWIITPRGDPMCIIPPR |
| | | GDPMWIIPPRGDPMCIIPPRGDP |
| 2547 | MAGE-like | DPMCIIPPRGDPMWIITPRGDPMCIIPPRGDPMWIIPP |
| | | RGDPMCIIPPRGDPMCIIPPRGDP |
| 2548 | MAGE-like | DPMWIITPRGDPMCIIPPRGDPMWIIPPRGDPMCIIPP |
| | | RGDPMCIIPPRGDPMCIIPPRGDP |
| 2549 | MAGE-like | DPMCIIPPRGDPMWIIPPRGDPMCIIPPRGDPMCIIPPR |
| | | GDPMCIIPPRGDPMCIIPPRGDP |
| 2550 | MAGE-like | DPMWIIPPRGDPMCIIPPRGDPMCIIPPRGDPMCIIPPR |
| | | GDPMCIIPPRGDPMCIIPPRGDP |
| 2551 | MAGE-like | DPMCIIPPRGDPMCIIPPRGDPMCIIPPRGDPMCIIPPR |
| | | GDPMCIIPPRGDPMCIIPPRGDP |
| 2552 | MAGE-like | DPMCIIPPRGDPMCIIPPRGDPMCIIPPRGDPMCIIPPR |
| | | GDPMCIIPPRGDPMCIIPPRGDP |
| 2553 | MAGE-like | DPMCIIPPRGDPMCIIPPRGDPMCIIPPRGDPMCIIPPR |
| | | GDPMCIIPPRGDPMCIIPPRGDP |
| 2554 | MAGE-like | DPMCIIPPRGDPMCIIPPRGDPMCIIPPRGDPMCIIPPR |
| | | GDPMCIIPPRGDPMCIIPPRGDP |
| 2555 | MAGE-like | DPMCIIPPRGDPMCIIPPRGDPMCIIPPRGDPMCIIPPR |
| | | GDPMCIIPPRGDPMCIIPPRGDP |
| 2556 | MAGE-like | DPMCIIPPRGDPMCIIPPRGDPMCIIPPRGDPMCIIPPR |
| | | GDPMCIIPPRGDPMCIIPPRGDP |
| 2557 | MAGE-like | DPMCIIPPRGDPMCIIPPRGDPMCIIPPRGDPMCIIPPR |
| | | GDPMCIIPPRGDPMCIIPPRGDP |
| 2558 | MAGE-like | DPMCIIPPRGDPMCIIPPRGDPMCIIPPRGDPMCIIPPR |
| | | GDPMCIIPPRGDPMCIIPPRGDP |
| 2559 | MAGE-like | DPMCIIPPRGDPMCIIPPRGDPMCIIPPRGDPMCIIPPR |
| | | GDPMCIIPPRGDPMCIIPPRGDP |
| 2560 | MAGE-like | DPMCIIPPRGDPMCIIPPRGDPMCIIPPRGDPMCIIPPR |
| | | GDPMCIIPPRGDPMCIIPPRGDP |
| 2561 | MAGE-like | DPMCIIPPRGDPMCIIPPRGDPMCIIPPRGDPMCIIPPR |
| | | GDPMCIIPPRGDPMCIIPPRGDP |
| 2562 | MAGE-like | DPMCIIPPRGDPMCIIPPRGDPMCIIPPRGDPMCIIPPR |
| | | GDPMCIIPPRGDPMWIIPPRGDP |
| 2563 | MAGE-like | DPMCIIPPRGDPMCIIPPRGDPMCIIPPRGDPMCIIPPR |
| | | GDPMWIIPPRGDPMWIIPPRGDP |
| 2564 | MAGE-like | DPMCIIPPRGDPMCIIPPRGDPMCIIPPRGDPMWIIPPR |
| | | GDPMWIIPPRGDPMCIIPPRGDP |
| 2565 | MAGE-like | DPMCIIPPRGDPMCIIPPRGDPMWIIPPRGDPMWIIPP |
| | | RGDPMCIIPPRGDPMCIIPPRGDP |
| 2566 | MAGE-like | DPMCIIPPRGDPMWIIPPRGDPMWIIPPRGDPMCIIPP |
| | | RGDPMCIIPPRGDPMCIIPPRGDP |
| 2567 | MAGE-like | DPMWIIPPRGDPMWIIPPRGDPMCIIPPRGDPMCIIPP |
| | | RGDPMCIIPPRGDPMCIIPPRGDP |
| 2568 | MAGE-like | DPMWIIPPRGDPMCIIPPRGDPMCIIPPRGDPMCIIPPR |
| | | GDPMCIIPPRGDPMCIIPPRGDP |
| 2569 | MAGE-like | DPMCIIPPRGDPMCIIPPRGDPMCIIPPRGDPMCIIPPR |
| | | GDPMCIIPPRGDPMCIIPPRGDP |
| 2570 | MAGE-like | DPMCIIPPRGDPMCIIPPRGDPMCIIPPRGDPMCIIPPR |
| | | GDPMCIIPPRGDPMCIIPPRGDP |
| 2571 | MAGE-like | DPMCIIPPRGDPMCIIPPRGDPMCIIPPRGDPMCIIPPR |
| | | GDPMCIIPPRGDPMCIIPPRGDP |
| 2572 | MAGE-like | DPMCIIPPRGDPMCIIPPRGDPMCIIPPRGDPMCIIPPR |
| | | GDPMCIIPPRGDPMCIIPPRGDP |
| 2573 | MAGE-like | DPMCIIPPRGDPMCIIPPRGDPMCIIPPRGDPMCIIPPR |
| | | GDPMCIIPPRGDPMCIIPPRGDP |
| 2574 | MAGE-like | DPMCIIPPRGDPMCIIPPRGDPMCIIPPRGDPMCIIPPR |
| | | GDPMCIIPPRGDPMCIIPPRGDP |
| | CBSL | PEDKEAKEPLWIRPDAPSRCTWQLGRPASESPHHHT |
| | | APAKSPKILPDILKKIGDTPMVRINK |
| 2575 | FBN1 | PPVLPVPPGFPPGPQIPVPRPPVEYLYPSREPPRVLPV |
| | | NVTDYCQLVRYLCQNGRCIPTPGS |
| | INPP5E_0 | PPEGRTLQGQLPGAPPAQRAGSPPDAPGSESPALAC |
| | | STPATPSGEDPPARAAPIAPRPPARP |
| | INPP5E_1 | LPGAPPAQRAGSPPDAPGSESPALACSTPATPSGEDP |
| | | PARAAPIAPRPPARPRLERALSLDD |
| 2576 | INPP5E_2 | PAQRAGSPPDAPGSESPALACSTPATPSGEDPPARAA |
| | | PIAPRPPARPRLERALSLDDKGWRR |
| | PEX1_0 | HLGKVWIPDDLRKRLNIEMHAVVRITPVEVTPKIPR |
| | | SLKLQPRENLPKDISEEDIKTVFYSW |
| 2577 | PEX1_1 | VVNQLLTQLDGVEGLQGVYVLAATSRPDLIDPALL |
| | | RPGRLDKCVYCPPPDQVSRLEILNVLS |
| | CAPRIN2 | EEQKKQETPKLWPVQLQKEQDPKKQTPKSWTPSM |
| | | QSEQNTTKSWTTPMCEEQDSKQPETPKS |
| | CBX4 | RCLSETHGEREPCKKRLTARSISTPTCLGGSPAAERP |
| | | ADLPPAAALPQPEVILLDSDLDEPI |
| | XDH | GDGNNPNCCMNQKKDHSVSLSPSLFKPEEFTPLDPT |
| | | QEPIFPPELLRLKDTPRKQLRFEGER |
| | EPAS1_0 | ATELRSHSTQSEAGSLPAFTVPQAAAPGSTTPSATSS |
| | | SSSCSTPNSPEDYYTSLDNDLKIEV |
| | EPAS1_1 | VPNDKFTQNPMRGLGHPLRHLPLPQPPSAISPGENS |
| | | KSRFPPQCYATQYQDYSLSSAHKVSG |
| | SHANK3_0 | GLVPPPEEFANGVLLATPLAGPGPSPTTVPSPASGKP |
| | | SSEPPPAPESAADSGVEEADTRSSS |
| | SHANK3_1 | GELTDTHTSFADGHTFLLEKPPVPPKPKLKSPLGKGP |
| | | VTFRDPLLKQSSDSELMAQQHHAAS |
| | ATF6 | PSAQPVLAVAGGVTQLPNHVVNVVPAPSANSPVNG |
| | | KLSVTKPVLQSTMRNVGSDIAVLRRQQ |
| | BCOR | KASNPEPSFKANENGLPPSSIFLSPNEAFRSPPIPYPRS |
| | | YLPYPAPEGIAVSPLSLHGKGPV |
| 2578 | CHD5 | PVPASPAHLLPAPLGLPDKMEAQLGYMDEKDPGAQ |
| | | KPRQPLEVQALPAALDRVESEDKHESP |
| | CCP110 | SDERGAHIMNSTCAAMPKLHEPYASSQCIASPNFGT |
| | | VSGLKPASMLEKNCSLQTELNKSYDV |
| 2579 | MMP9_0 | LMYPMYRFTEGPPLHKDDVNGIRHLYGPRPEPEPRP |
| | | PTTTTPQPTAPPTVCPTGPPTVHPSE |
| | MMP9_1 | GPPLHKDDVNGIRHLYGPRPEPEPRPPTTTTPQPTAP |
| | | PTVCPTGPPTVHPSERPTAGPTGPP |
| | BCL11B_0 | LNPMAIDSPAMDFSRRLRELAGNSSTPPPVSPGRGN |
| | | PMHRLLNPFQPSPKSPFLSTPPLPPM |
| | BCL11B_1 | AGNSSTPPPVSPGRGNPMHRLLNPFQPSPKSPFLSTP |
| | | PLPPMPPGGTPPPQPPAKSKSCEFC |
| | BCL11B_2 | TPPPVSPGRGNPMHRLLNPFQPSPKSPFLSTPPLPPMP |
| | | PGGTPPPQPPAKSKSCEFCGKTFK |
| | BCL11B_3 | PMHRLLNPFQPSPKSPFLSTPPLPPMPPGGTPPPQPPA |
| | | KSKSCEFCGKTFKFQSNLIVHRRS |
| | RB1 | DSIIVFYNSVFMQRLKTNILQYASTRPPTLSPIPHIPRS |
| | | PYKFPSSPLRIPGGNIYISPLKS |
| | AHSG_0 | GAEVAVTCMVFQTQPVSSQPQPEGANEAVPTPVVD |
| | | PDAPPSPPLGAPGLPPAGSPPDSHVLL |
| | AHSG_1 | GANEAVPTPVVDPDAPPSPPLGAPGLPPAGSPPDSH |
| | | VLLAAPPGHQLHRAHYDLRHTFMGVV |
| | TCTN3 | TDGGTLQSPSEATATRPAVPGLPTVVPTLVTPSAPG |
| | | NRTVDLFPVLPICVCDLTPGACDINC |
| | NR2F2 | QDEVPGSQGSQASQAPPVPGPPPGAPHTPQTPGQGG |
| | | PASTPAQTAAGGQGGPGGPGSDKQQQ |
| | KHDRBS1 | PSVRQTPSRQPPLPHRSRGGGGGSRGGARASPATQP |
| | | PPLLPPSATGPDATVGGPAPTPLLPP |
| | ARRB1 | SSDVAVELPFTLMHPKPKEEPPHREVPENETPVDTN |
| | | LIELDTNDDDIVFEDFARQRLKGMKD |
| | TFAP2B | HDGVPSHSSRLSQLGSVSQGPYSSAPPLSHTPSSDFQ |
| | | PPYFPPPYQPLPYHQSQDPYSHVND |
| 2580 | ASPH | DVDDAKVLLGLKERSTSEPAVPPEEAEPHTEPEEQV |
| | | PVEAEPQNIEDEAKEQIQSLLHEMVH |
| | KSR2_0 | IQWPTTETGKENNPVCPPEPTPWIRTHLSQSPRVPSK |
| | | CVQHYCHTSPTPGAPVYTHVDRLTV |
| | KSR2_1 | RSLPPSPRQRHAVRTPPRTPNIVTTVTPPGTPPMRKK |
| | | NKLKPPGTPPPSSRKLIHLIPGFTA |
| | KSR2_2 | RQQKNFNLPASHYYKYKQQFIFPDVVPVPETPTRAP |
| | | QVILHPVTSNPILEGNPLLQIEVEPT |
| | TNS1_0 | SGYIPSGHSLGTPEPAPRASLESVPPGRSYSPYDYQP |
| | | CLAGPNQDFHSKSPASSSLPAFLPT |
| | TNS1_1 | LPAFLPTTHSPPGPQQPPASLPGLTAQPLLSPKEATS |
| | | DPSRTPEEEPLNLEGLVAHRVAGVQ |
| | TNS1_2 | SASGYQAPSTPSFPVSPAYYPGLSSPATSPSPDSAAF |
| | | RQGSPTPALPEKRRMSVGDRAGSLP |
| | ZEB2 | SNSRSPSLERSSKPLAPNSNPPTKDSLLPRSPVKPMD |
| | | SITSPSIAELHNSVTNCDPPLRLTK |
| | CREBBP_0 | QGQVPGAALPNPLNMLGPQASQLPCPPVTQSPLHPT |
| | | PPPASTAAGMPSLQHTTPPGMTPPQP |
| | CREBBP_1 | GAALPNPLNMLGPQASQLPCPPVTQSPLHPTPPPAST |
| | | AAGMPSLQHTTPPGMTPPQPAAPTQ |
| | CREBBP_2 | AQLMRRRMATMNTRNVPQQSLPSPTSAPPGTPTQQ |
| | | PSTPQTPQPPAQPQPSPVSMSPAGFPS |
| | CREBBP_3 | MATMNTRNVPQQSLPSPTSAPPGTPTQQPSTPQTPQ |
| | | PPAQPQPSPVSMSPAGFPSVARTQPP |
| | CREBBP_4 | MNTRNVPQQSLPSPTSAPPGTPTQQPSTPQTPQPPAQ |
| | | PQPSPVSMSPAGFPSVARTQPPTTV |
| | CREBBP_5 | GQQIATSLSNQVRSPAPVQSPRPQSQPPHSSPSPRIQP |
| | | QPSPHHVSPQTGSPHPGLAVTMAS |
| | CREBBP_6 | QVRSPAPVQSPRPQSQPPHSSPSPRIQPQPSPHHVSPQ |
| | | TGSPHPGLAVTMASSIDQGHLGNP |
| | CREBBP_7 | APVQSPRPQSQPPHSSPSPRIQPQPSPHHVSPQTGSPH |
| | | PGLAVTMASSIDQGHLGNPEQSAM |
| 2581 | ARHGEF10L | SAALGVPSLAPERDTDPPLIHLDSIPVTDPDPAAAPP |
| | | GTGVPAWVSNGDAADAAFSGARHSS |
| 2582 | KAT8 | EGEPGPGENAAAEGTAPSPGRVSPPTPARGEPEVTV |
| | | EIGETYLCRRPDSTWHSAEVIQSRVN |
| 2583 | GBF1_0 | PSALWEITWERIDCFLPHLRDELFKQTVIQDPMPME |
| | | PQGQKPLASAHLTSAAGDTRTPGHPP |
| | GBF1_1 | IPSELGACDFEKPESPRAASSSSPGSPVASSPSRLSPTP |
| | | DGPPPLAQPPLILQPLASPLQVG |
| | GBF1_2 | GACDFEKPESPRAASSSSPGSPVASSPSRLSPTPDGPP |
| | | PLAQPPLILQPLASPLQVGVPPMT |
| | GBF1_3 | CDFEKPESPRAASSSSPGSPVASSPSRLSPTPDGPPPL |
| | | AQPPLILQPLASPLQVGVPPMTLP |
| | ESRP2 | QATPTLIPTETAALYPSSALLPAARVPAAPTPVAYYP |
| | | GPATQLYLNYTAYYPSPPVSPTTVG |
| | FGFR1 | PYWTSPEKMEKKLHAVPAAKTVKFKCPSSGTPNPT |
| | | LRWLKNGKEFKPDHRIGGYKVRYATWS |
| | FNDC1_0 | IVAMPTTSKADVEQNTEDNGKPEKPEPSSPSPRAPAS |
| | | SQHPSVPASPQGRNAKDLLLDLKNK |
| 2584 | FNDC1_1 | GHAASPARPSRPGGPQSRARVPSRAAPGKSEPPSKR |
| | | PLSSKSQQSVSAEDDEEEDAGFFKGG |
| | LCAT | PWQWVTLLLGLLLPPAAPFWLLNVLFPPHTTPKAEL |
| | | SNHTRPVILVPGCLGNQLEAKLDKPD |
| 2585 | COL4A5_0 | RSGVPGLKGDDGLQGQPGLPGPTGEKGSKGEPGLP |
| | | GPPGPMDPNLLGSKGEKGEPGLPGIPG |
| 2586 | COL4A5_1 | LLGSKGEKGEPGLPGIPGVSGPKGYQGLPGDPGQPG |
| | | LSGQPGLPGPPGPKGNPGLPGQPGLI |
| | COL4A5_2 | IKGSVGDPGLPGLPGTPGAKGQPGLPGFPGTPGPPGP |
| | | KGISGPPGNPGLPGEPGPVGGGGHP |
| | FGD1 | PGQSLEPHPEGPQRLRSDPGPPTETPSQRPSPLKRAP |
| | | GPKPQVPPKPSYLQMPRMPPPLEPI |
| | PIK3R1 | IGWLNGYNETTGERGDFPGTYVEYIGRKKISPPTPKP |
| | | RPPRPLPVAPGSSKTEADVEQQALT |
| 2587 | RELA | TGPGWEARGSFSQADVHRQVAIVFRTPPYADPSLQ |
| | | APVRVSMQLRRPSDRELSEPMEFQYLP |
| | EP300_0 | MQIQRAAETQRQMAHVQIFQRPIQHQMPPMTPMAP |
| | | MGMNPPPMTRGPSGHLEPGMGPTGMQQ |
| | EP300_1 | GQQIPNSLSNQVRSPQPVPSPRPQSQPPHSSPSPRMQ |
| | | PQPSPHHVSPQTSSPHPGLVAAQAN |
| | EP300_2 | QVRSPQPVPSPRPQSQPPHSSPSPRMQPQPSPHHVSP |
| | | QTSSPHPGLVAAQANPMEQGHFASP |
| | EP300_3 | QPVPSPRPQSQPPHSSPSPRMQPQPSPHHVSPQTSSP |
| | | HPGLVAAQANPMEQGHFASPDQNSM |
| 2588 | FEN1 | SIEEIVRRLDPNKYPVPENWLHKEAHQLFLEPEVLDP |
| | | ESVELKWSEPNEEELIKFMCGEKQF |
| | FOXM1_0 | VGGLDFSPVQTSQGASDPLPDPLGLMDLSTTPLQSA |
| | | PPLESPQRLLSSEPLDLISVPFGNSS |
| | FOXM1_1 | TSQGASDPLPDPLGLMDLSTTPLQSAPPLESPQRLLS |
| | | SEPLDLISVPFGNSSPSDIDVPKPG |
| | ACD | ICSAPATLTPRSPHASRTPSSPLQSCTPSLSPRSHVPSP |
| | | HQALVTRPQKPSLEFKEFVGLPC |
| 2589 | SON_0 | DSYTDTYTEAYMVPPLPPEEPPTMPPLPPEEPPMTPP |
| | | LPPEEPPEGPALPTEQSALTAENTW |
| | SON_1 | SETAETFDSMRASGHVASEVSTSLLVPAVTTPVLAE |
| | | SILEPPAMAAPESSAMAVLESSAVTV |
| | HTT_0 | INICAHVLDDVAPGPAIKAALPSLTNPPSLSPIRRKGK |
| | | EKEPGEQASVPLSPKKGSEASAAS |
| 2590 | HTT_1 | VAPGPAIKAALPSLTNPPSLSPIRRKGKEKEPGEQAS |
| | | VPLSPKKGSEASAASRQSDTSGPVT |
| | PHLPP1 | APGAFGGPPRAPPADLPLPVGGPGGWSRRASPAPSD |
| | | SSPGEPFVGGPVSSPRAPRPVVSDTE |
| | NAF1 | DFGVGEGPAAPSPGSAPVPGTQPPLQSFEGSPDAGQ |
| | | TVEVKPAGEQPLQPVLNAVAAGTPAP |
| | ERBB2 | PSETDGYVAPLTCSPQPEYVNQPDVRPQPPSPREGPL |
| | | PAARPAGATLERPKTLSPGKNGVVK |
| 2591 | DAZAP1_0 | RGFGFVKFKDPNCVGTVLASRPHTLDGRNIDPKPCT |
| | | PRGMQPERTRPKEGWQKGPRSDNSKS |
| | DAZAP1_1 | VKFKDPNCVGTVLASRPHTLDGRNIDPKPCTPRGM |
| | | QPERTRPKEGWQKGPRSDNSKSNKIFV |
| 2592 | SMAD3 | PRHTEIPAEFPPLDDYSHSIPENTNFPAGIEPQSNIPET |
| | | PPPGYLSEDGETSDHQMNHSMDA |
| | E2F8 | VAPLDPPVNAEMELTAPSLIQPLGMVPLIPSPLSSAV |
| | | PLILPQAPSGPSYAIYLQPTQAHQS |
| 2593 | SQSTM1 | WTHLSSKEVDPSTGELQSLQMPESEGPSSLDPSQEG |
| | | PTGLKEAALYPHLPPEADPRLIESLS |
| | PC_0 | RPAQNRAQKLLHYLGHVMVNGPTTPIPVKASPSPTD |
| | | PVVPAVPIGPPPAGFRDILLREGPEG |
| 2594 | PC_1 | RAQKLLHYLGHVMVNGPTTPIPVKASPSPTDPVVPA |
| | | VPIGPPPAGFRDILLREGPEGFARAV |
| | TMIGD2 | QSIYSTSFPQPAPRQPHLASRPCPSPRPCPSPRPGHPV |
| | | SMVRVSPRPSPTQQPRPKGFPKVG |
| | MAPT_0 | PAKTPPAPKTPPSSGEPPKSGDRSGYSSPGSPGTPGS |
| | | RSRTPSLPTPPTREPKKVAVVRTPP |
| | MAPT_1 | PPSSGEPPKSGDRSGYSSPGSPGTPGSRSRTPSLPTPP |
| | | TREPKKVAVVRTPPKSPSSAKSRL |
| | MAPT_2 | EPPKSGDRSGYSSPGSPGTPGSRSRTPSLPTPPTREPK |
| | | KVAVVRTPPKSPSSAKSRLQTAPV |
| 2595 | MAPT_3 | GDRSGYSSPGSPGTPGSRSRTPSLPTPPTREPKKVAV |
| | | VRTPPKSPSSAKSRLQTAPVPMPDL |
| | KCNQ2_0 | LIPPLNQLELLRNLKSKSGLAFRKDPPPEPSPSKGSPC |
| | | RGPLCGCCPGRSSQKVSLKDRVFS |
| | KCNQ2_1 | NQLELLRNLKSKSGLAFRKDPPPEPSPSKGSPCRGPL |
| | | CGCCPGRSSQKVSLKDRVFSSPRGV |
| | MBNL2 | SFAPYLAPVTPGVGLVPTEILPTTPVIVPGSPPVTVPG |
| | | STATQKLLRTDKLEVCREFQRGNC |
| 2593 | SCARA3 | LRGAPGPPGPRGFKGDMGVKGPVGGRGPKGDPGSL |
| | | GPLGPQGPQGQPGEAGPVGERGPVGPR |
| | FN1 | PGTSGQQPSVGQQMIFEEHGFRRTTPPTTATPIRHRP |
| | | RPYPPNVGEEIQIGHIPREDVDYHL |
| | KLF5_0 | TAVKQFQGMPPCTYTMPSQFLPQQATYFPPSPPSSEP |
| | | GSPDRQAEMLQNLTPPPSYAATIAS |
| 2597 | KLF5_1 | FQGMPPCTYTMPSQFLPQQATYFPPSPPSSEPGSPDR |
| | | QAEMLQNLTPPPSYAATIASKLAIH |
| | uncharacterized_ | VIRALGPLVPPTEGGLWSDQVSWPLWEDVKTPEPG |
| | LOC101060588_0 | EPGSPLPASPHPPLQPPAFPDPPIRSP |
| 2598 | uncharacterized_ | GPLVPPTEGGLWSDQVSWPLWEDVKTPEPGEPGSP |
| | LOC101060588_1 | LPASPHPPLQPPAFPDPPIRSPDPAVS |
| 2599 | uncharacterized | WEDVKTPEPGEPGSPLPASPHPPLQPPAFPDPPIRSPD |
| | LOC101060588_2 | PAVSSAHSFPAPRLAWSCVLHSPL |
| | uncharacterized_ | TPEPGEPGSPLPASPHPPLQPPAFPDPPIRSPDPAVSSA |
| | LOC101060588_3 | HSFPAPRLAWSCVLHSPLSLPLS |
| | translation_initiation_ | PGSLLPTPASLWQAQCPRHMHSWSSAPGRLTPHPPG |
| | factor_IF-2-like | PAPGTKLATGATSSACSRPQGRPCPQ |
| | putative_uncharacterized | SAQAGPPETAHAADPQPRGPQAPPRLPPSLSPERVHP |
| | protein_MGC34800 | GQPAAPAEPAPGAPALRSGPSQPRG |
| | uncharacterized_ | SLPWPLRAAPLYAGRSGQGGEPGARAPRQGTPEPG |
| | LOC100507221 | ELDQERPPAPPEQGRRAAAAVAKSGGG |
| 2600 | basic_proline- | KEPAQATRPPRTPLRPPGLLGPRSGHPASSDPAQATR |
| | rich_protein-like_0 | PPRTPQNTPKAHGRLLTVRTGWESF |
| | basic_proline- | SAGNKENARTWRRSEGGLAGPPLAKAPRSHSPPGC |
| | rich_protein-like_1 | SPHGQSLPPRRRTPPSQLTGSARSRRP |
| | basic_proline- | ENARTWRRSEGGLAGPPLAKAPRSHSPPGCSPHGQS |
| | rich_protein-like_2 | LPPRRRTPPSQLTGSARSRRPGSPFR |
| | basic_proline- | RSPGAGGVQGGGAGGIPAPRAPRPPPSGAPSPTHVE |
| | rich_protein-like_3 | PPRPRRPAPTREGTRASPHTRASRSR |
| | uncharacterized_ | CWDSHLPFRKKGAAPAPGCGDRIDTVPTSATPNGRT |
| | LOC107987269 | PGRGALLAAPILSQPCHFQSCQHPSQ |
| | sine oculis- | GCLSKGSQRSLTPSWSPSVSPGSEADSSWGTPSTPPR |
| | binding protein_ | PHSPPSLPRPSPSPWVQARPGIPPP |
| | homolog_0 | |
| | sine_oculis- | SPGSEADSSWGTPSTPPRPHSPPSLPRPSPSPWVQAR |
| | binding_protein_ | PGIPPPSEQTLFKGLWRLEGIEPPP |
| | homolog_1 | |
| 2601 | uncharacterized_ | LAMLLGRAVGTRVGQAPCPALGLSFFIDAAEPGGPP |
| | LOC107987285_0 | PELCIPLGVTHGRGQPLGHCAFTGDG |
| 2602 | uncharacterized_ | LSAAVVFHRLTEAGLTRAEIHPSVYSPTSFEPQPTQT |
| | LOC107987285_1 | HGGGTNALKPRAMIHNEDTEHFRHP |
| | mucin-1-like_0 | PAGSPAAPLQTATSVPPWVSSCTTSNCNISSPLGLQQ |
| | | HGPQPGTSAPPNPGLQLHSPQPGTS |
| | mucin-1-like_1 | NCNISSPLGLQQHGPQPGTSAPPNPGLQLHSPQPGTS |
| | | APPNPGLQLHGPQTGTSAPCRVSSC |

### Small Molecule Inhibitors

In some embodiments, the current invention provides an inhibitor of DNA-speckle association which is a small molecule that mimics the key chemistry of the peptide inhibitor. These features are determined based on the optimization of the speckle-targeting portion of the peptide inhibitor, and includes features that mimic the kinks that are a feature of Proline-containing peptides as well as the negatively charged components at particular locations of the molecule.

### Using the Speckle Signature as a Prognostic Tool

In some embodiments the speckle signature expressed by cells, including cancer cells, is used as a prognostic or diagnostic tool in order to determine patient prognosis, as well as to identify cancers which would benefit from treatments that alter speckle regulated gene expression such as the polypeptides and compositions of the present invention. The data disclosed herein indicate that speckle signature divides clear cell renal cell carcinoma and neuroblastoma patients into distinct subclasses that differ in survival rates, and in the key molecular features of clear cell renal cell carcinoma. The same speckle signature is present in 24 of the 30 adult cancer types examined, and predicted patient survival of other cancer types depending on mutation status, being predictive of survival in: melanoma with wild type KMT2D, thyroid cancer with wild type BRAF, endometrial cancer with mutant PIK3R1, and lung adenocarcinoma with mutant TTN. In the case of lung adenocarcinoma, splitting cancers by speckle signature enables prediction of patient survival based on p53 mutation status. Hence the speckle signature can be used in the clinic to identify high-risk patient groups and prioritize them for specific targeted therapies, including the polypeptides and compositions of the present invention, recently FDA-approved HIF2A inhibitors, tyrosine kinase inhibitors, immunotherapy, and any routinely used treatment employed in each respective cancer type.

Gene expression readouts of speckle signature: The speckle signature can be determined from genome-wide RNA expression data of groups of patient samples or from expression analysis of the minimal speckle signature, consisting of 18 speckle protein genes (FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, and EPC2). This minimal speckle signature represents the overlap between the 16 different cancer types, and is sufficient to separate tumor samples into the two speckle signature groups. Speckle gene expression from genome wide or the minimal speckle signature can then be used to generate a speckle score that provides a quantitative value to the speckle score, using the following method:
1. Getting the Z-score of each speckle protein gene in a group of patients
2. For each Signature I speckle protein gene, divide its Z-score by the number of speckle protein genes in speckle Signature I, then take the sum of all these values for Signature I speckle protein genes.
3. For each Signature II speckle protein gene, divide its Z-score by the number of speckle protein genes in speckle Signature II, then take the sum of all these values for Signature II speckle protein genes.
4. Take the log(2) of the ratio of the result from Step 2 to the result from Step 3. In the calculated speckle score, samples with high positive values are strongly Signature I and samples with low negative values are strongly Signature II.

Further development of gene expression readouts of speckle signature involves bioinformatic identification the minimal number of genes needed to assign a tumor sample to speckle Signature I or Signature II. This process incorporates gene expression read-outs of non-speckle protein genes that are highly correlated with the speckle score, including, but not limited to GADD45GIP1 (readout of Signature I) and LATS1 (readout of Signature II). Gene expression readouts of speckle signature can include RNA or protein measurements of gene expression.

### Readouts of Speckle Signature

In some embodiments, the current invention provides methods for determining the speckle signature of a particular tissue or tumor sample. The level of one or more speckle signature genes is measured in the sample. In some embodiments, the sample is a tissue sample that includes a tumor cell, for example, from a biopsy or formalin-fixed, paraffin-embedded (FFPE) sample. Exemplary test samples also include body fluids (e.g. blood, serum, plasma, amniotic fluid, sputum, urine, cerebrospinal fluid, lymph, tear fluid, feces, or gastric fluid), tissue extracts, and culture media (e.g., a liquid in which a cell, such as a pathogen cell, has been grown). If desired, the sample is purified prior to detection using any standard method typically used for isolating nucleic acid molecules from a biological sample.

In some embodiments, the expression levels of speckle signature genes are determined using imaging-based immunofluorescence methods of detecting speckle signature. Here, the expression of SON protein expression and location is assessed. SON is a speckle-associated protein that has been found to be required for speckle organization and structure. Visualization of SON protein enables the visualization of speckle structure and positioning within the nucleus. This method of visualization can be applied to FFPE tumor tissue sections, which are frequently collected in the clinic to assess tumor pathology. In some embodiments, the determination of speckle signature can be accomplished by means for analyzing multiple types of nucleic acids or proteins present in a sample, including DNA and RNA. In various embodiments, sample preparation involves extracting a mixture of nucleic acid molecules (e.g., DNA and RNA). In some embodiments, the radial position of speckles in the nucleus are correlated with speckle signature score. For example, more centralized speckle formation is associated with speckle signature II, and speckle signature II RNA expression patterns. Likewise, more diffuse or less centralized speckle expression correlates with speckle signature I and speckle signature I RNA expression patterns.

The expression levels of speckle signature genes can be detected by any suitable method. The methods described herein can be used individually or in combination for a more accurate detection of the speckle signature genes. Methods for conducting polynucleotide hybridization assays have been developed in the art. Hybridization assay procedures and conditions will vary depending on the application and are selected in accordance with the general binding methods known including those referred to in: Sambrook and Russell, Molecular Cloning: A Laboratory Manual (3^{rd} Ed. Cold Spring Harbor, N.Y, 2001); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623. A data analysis algorithm (E-predict) for interpreting the hybridization results from an array is publicly available (see Urisman, 2005, Genome Biol 6:R78).

The term “speckle signature” as used herein refers to the reproducible reciprocal expression pattern of nuclear speckle protein genes as determined by analysis of human tumor RNA-seq datasets.

The term “speckle signature I” refers to the speckle signature with generally higher levels, compared to the cohort average, of speckle protein genes: VAX2, JDP2, PLEKHN1, HDAC5, C11ORF49, SLC4A2, STYXL1, TMEM179B, TAB1, ZNF446, TBXA2R, UNC45A, PCBP1, PHLDB3, KTI12, AKAP17A, PRCC, ZNF821, SPINDOC, HSF4, DEXI, HEXIM2, EHMT2, VPS72, DDX39A, KIF22, DPCD, LHPP, CD2BP2, CDK11B, GTF2H4, DGKZ, SARNP, ALYREF, SLC2A4RG, TEPSIN, AKAP8L, PPIE, STK19, FIBP, C60RF226, H2AFX, EGFL8, PSMD13, CACTIN, EXOSC7, C120RF57, THAP4, TMEM259, THOC6, AP5Z1, PQBP1, RBM10, C1ORF35, C19ORF24, SART1, CDC34, FASTK, POMP, PRPF6, PRPF19, BRK1, UFC1, SNRPA1, ZCCHC17, SNRPB2, PCP2, SSH3, SETD1A, WDR90, THEM6, U2AF2, RBM14, MAST3, LIMK1, SF3B4, DDX39B, RTEL1, ZNF165, MAPK12, PSMD8, CDK5RAP1, PDZK1IP1, SETD4, CHTOP, CDK11A, SRSF4, TBX19, RTN2, CCDC32, CYSRT1, IQCK, MPP1, MAMSTR, ILRUN, DBNDD1, EPHB6, TCF15, C60RF52, CYGB, CCDC85C, PHYHD1, ITPKC, CDC25C, RMI2, SNRNP40, HISTIHIE, ZC3H18, and generally lower levels, compared to the cohort average, of speckle protein genes: SON, RBM27, TCF12, BCLAF1, ERBIN, SETD2, TCP11L2, EPC2, TRIP12, YLPM1, LMTK2, GPATCH8, DDX46, PRPF4B, TAB3, EPG5, RSBN1L, SF3B1, PUS7L, KCTD20, RBM26, BAZ2A, RBM41, RREB1, ZNF621, FAM160B1, CDK13, SDE2, DHX15, PRPF40A, CHIC1, SREK1, LIN52, BARD1, ZNF441, GNAQ, THRAP3, HBP1, SMC5, PPP4R3B, RBBP6, TTC26, COG6, ZC3H14, UBE3B, MRTFB, YTHDF3, UBE4A, CBLL1, API5, CMTR2, TBC1D12, WRN, KIAA1328, TMEM209, ZCCHC4, MAPK14, ZNF160, SLU7, ERCC8, FOXJ3, PCLO, RSRC1, ZC3H11A, BMP2K, RALGAPB, FBXL4, RTL6, RCAN3, FBXO34, ZBTB8A, CWF19L2, SRRM2, HELQ, FYTTD1, PPIG, ANKRD44, SOCS6, S100PBP, ZNF304, ZNF543, RBM25, EFCAB13, CPD, ARMCX5, POLI, ZNF551, MAML3, POLR3B, SFMBT2, DDX17, RNF169, KAT6A, DDX42, GPATCH2, CBFA2T2, E2F3, ZNF169, TAF5L, KIAA0100, PRKAA1, LHX4, RSRC2, CSRNP2, NCBP3, NCAPG2, SF3A1, DENND1B, BRD2, PNISR, E2F7, LRRC8B, PACSIN2, PNN, KIAA0556, SAP130, CPSF6, MAP3K7, TADA2A, HP1BP3, ZNF217, BRD1, SRRM1, SRSF11, GLYR1, FAM227B, AAGAB, PLRG1, FCHSD2, MECOM, TMEM56, CDYL, ELOA, STK17A, RIOK1, ARHGAP42, R3HCC1L, COPS4, BORCS7, THOC1, CIR1, PYROXD1, ARHGAP18, NSL1, WTAP, ZNHIT6, BCAS2, HAUS6, MORF4L1, SMC4, MBD4, PRPF18, CWC22, UBAP2L, SMURF2, KDM6B, PRKAA2, LIFR, RBM8A, SNURF, DAZAP2, FAM120C, WDR17, ZDHHC15, GTF2H2C, SRGAP1, ZSWIM5, RAF1, ZNF286B, ZNF528, ZNF572, ZNF527, XYLB, FNBP4, PRPF4, SIPA1L3, ZNF382, RFXAP, RBM39, CWC25, ZIM2, ANXA9, MFSD11, BPNT1, GPN3, MAPT, PPP1R16B, ZNF250, RAD52, ZNF786, GNB5, MNS1, TARBP1, RBM6, PRKN, ZCWPW2, MAMDC2, IPCEF1, NFATC4, LPAR1, VXN, FAM107A, IL16, USP22, RNF112, CRY2, PLAGI, IQUB, PPP1R8, BNIP3L, or any combination thereof. Not all of the speckle protein genes will be expressed, and not all of them will completely fit in with the rest of the signature. The speckle signature rather refers to the general pattern of expression of the group of speckle protein genes, as can be observed. speckle signature I, as defined herein, is the reciprocal of speckle signature II.

The term “speckle signature II” refers to the speckle signature with generally higher levels, compared to the cohort average, of speckle protein genes: SON, RBM27, TCF12, BCLAF1, ERBIN, SETD2, TCP11L2, EPC2, TRIP12, YLPM1, LMTK2, GPATCH8, DDX46, PRPF4B, TAB3, EPG5, RSBN1L, SF3B1, PUS7L, KCTD20, RBM26, BAZ2A, RBM41, RREB1, ZNF621, FAM160B1, CDK13, SDE2, DHX15, PRPF40A, CHIC1, SREK1, LIN52, BARD1, ZNF441, GNAQ, THRAP3, HBP1, SMC5, PPP4R3B, RBBP6, TTC26, COG6, ZC3H14, UBE3B, MRTFB, YTHDF3, UBE4A, CBLL1, API5, CMTR2, TBC1D12, WRN, KIAA1328, TMEM209, ZCCHC4, MAPK14, ZNF160, SLU7, ERCC8, FOXJ3, PCLO, RSRC1, ZC3H11A, BMP2K, RALGAPB, FBXL4, RTL6, RCAN3, FBXO34, ZBTB8A, CWF19L2, SRRM2, HELQ, FYTTD1, PPIG, ANKRD44, SOCS6, S100PBP, ZNF304, ZNF543, RBM25, EFCAB13, CPD, ARMCX5, POLI, ZNF551, MAML3, POLR3B, SFMBT2, DDX17, RNF169, KAT6A, DDX42, GPATCH2, CBFA2T2, E2F3, ZNF169, TAF5L, KIAA0100, PRKAA1, LHX4, RSRC2, CSRNP2, NCBP3, NCAPG2, SF3A1, DENND1B, BRD2, PNISR, E2F7, LRRC8B, PACSIN2, PNN, KIAA0556, SAP130, CPSF6, MAP3K7, TADA2A, HP1BP3, ZNF217, BRD1, SRRM1, SRSF11, GLYR1, FAM227B, AAGAB, PLRG1, FCHSD2, MECOM, TMEM56, CDYL, ELOA, STK17A, RIOK1, ARHGAP42, R3HCC1L, COPS4, BORCS7, THOC1, CIR1, PYROXD1, ARHGAP18, NSL1, WTAP, ZNHIT6, BCAS2, HAUS6, MORF4L1, SMC4, MBD4, PRPF18, CWC22, UBAP2L, SMURF2, KDM6B, PRKAA2, LIFR, RBM8A, SNURF, DAZAP2, FAM120C, WDR17, ZDHHC15, GTF2H2C, SRGAP1, ZSWIM5, RAF1, ZNF286B, ZNF528, ZNF572, ZNF527, XYLB, FNBP4, PRPF4, SIPA1L3, ZNF382, RFXAP, RBM39, CWC25, ZIM2, ANXA9, MFSD11, BPNT1, GPN3, MAPT, PPP1R16B, ZNF250, RAD52, ZNF786, GNB5, MNS1, TARBP1, RBM6, PRKN, ZCWPW2, MAMDC2, IPCEF1, NFATC4, LPAR1, VXN, FAM107A, IL16, USP22, RNF112, CRY2, PLAGI, IQUB, PPP1R8, BNIP3L, and generally lower levels, compared to the cohort average, of speckle protein genes: VAX2, JDP2, PLEKHN1, HDAC5, C11ORF49, SLC4A2, STYXL1, TMEM179B, TAB1, ZNF446, TBXA2R, UNC45A, PCBP1, PHLDB3, KTIl2, AKAP17A, PRCC, ZNF821, SPINDOC, HSF4, DEXI, HEXIM2, EHMT2, VPS72, DDX39A, KIF22, DPCD, LHPP, CD2BP2, CDK11B, GTF2H4, DGKZ, SARNP, ALYREF, SLC2A4RG, TEPSIN, AKAP8L, PPIE, STK19, FIBP, C60RF226, H2AFX, EGFL8, PSMD13, CACTIN, EXOSC7, C120RF57, THAP4, TMEM259, THOC6, AP5Z1, PQBP1, RBM10, C1ORF35, C19ORF24, SART1, CDC34, FASTK, POMP, PRPF6, PRPF19, BRK1, UFC1, SNRPA1, ZCCHC17, SNRPB2, PCP2, SSH3, SETD1A, WDR90, THEM6, U2AF2, RBM14, MAST3, LIMK1, SF3B4, DDX39B, RTEL1, ZNF165, MAPK12, PSMD8, CDK5RAP1, PDZK1IP1, SETD4, CHTOP, CDK11A, SRSF4, TBX19, RTN2, CCDC32, CYSRT1, IQCK, MPP1, MAMSTR, ILRUN, DBNDD1, EPHB6, TCF15, C60RF52, CYGB, CCDC85C, PHYHD1, ITPKC, CDC25C, RMI2, SNRNP40, HISTIHIE, ZC3H18 or any combination thereof. Depending on the context, not all the speckle protein genes will be expressed, and not all of them will completely fit in with the rest of the signature. The speckle signature rather refers to the general pattern of expression of the group of speckle protein genes. speckle signature II, as defined herein, is the reciprocal of speckle signature I.

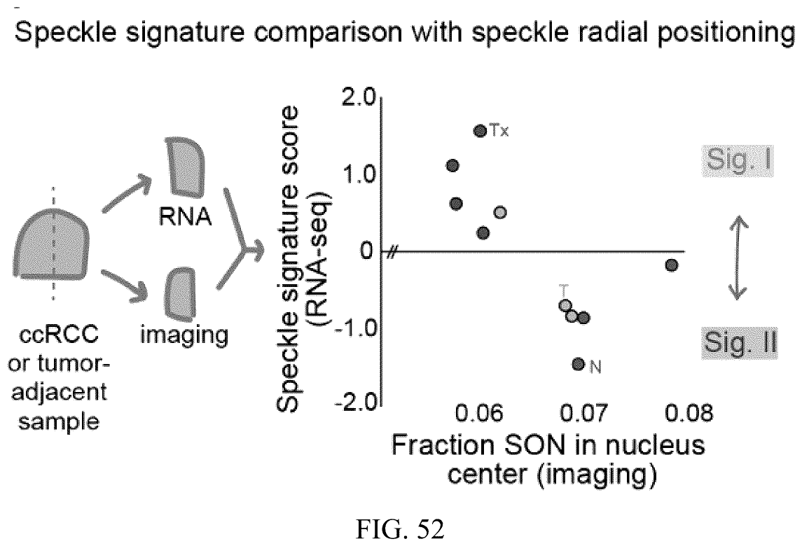

In some embodiments, the radial positioning of the speckle structures also correlates to speckle signature. In some embodiments, a SON signal being more central corresponds to the speckle Signature II RNA expression pattern; SON signal being less central corresponds to the Signature I RNA expression pattern, as per FIG. 52

### Methods

In some embodiments, the current invention provides a method for inhibiting transcription factor/DNA-speckle association in a cell, comprising contacting the cell with an effect amount of an inhibitor of transcription factor/DNA-speckle association. In some embodiments, the inhibitor is a polypeptide comprising a first polypeptide domain, a second polypeptide domain, and a third polypeptide domain, wherein the first polypeptide domain comprises a cell penetrating peptide, the second polypeptide domain comprises a linker region, and the third polypeptide domain comprises a DNA-speckle targeting motif. In some embodiments, the DNA-speckle targeting motif comprises an amino acid sequence set forth in any one of SEQ ID NOs: 1-2602. In some embodiments, the inhibitor is a small molecule. In some embodiments, the inhibitor is a combination of a small molecule and a polypeptide comprising one or more of the polypeptides set for in SEQ ID NOs: 1-2602.

In some embodiments, the invention includes a method of generating inhibitors of DNA speckle association, comprising screening a library of protein sequences for those comprising a DNA-speckle targeting motif as identified by the following rules:
1. The sequence comprises the pattern X1(30)-X2-P-X1(30), wherein X1 is any amino acid and X2 is an amino acid selected from T, S, E, or D.
2. The sequence may be the full 62 contiguous amino acid sequence, or truncated versions therein.
3. The sequence does not comprise four or more consecutive proline residues.
4. The sequence contains proline residues in a minimum of three of positions 16, 21, 36, 41, or 46.
5. The sequence comprises at least five negative or phosphorylatable amino acids selected from the group consisting of D, E, T, and S.
6. The sequence comprises at least five small or hydrophobic amino acids selected from the group consisting of A, M, V, F, L, and I.
7. The sequence comprises fewer than fifteen positively charged amino acids selected from the group consisting of R, H, and K.

The protein sequences which comprise the DNA-speckle targeting motif are then synthesized as distinct inhibitor peptides, which can then be administered to a cell or a subject in need thereof to disrupt the target protein's association with DNA-speckles thereby achieving inhibition. In some embodiments, the inhibitor peptides are further modified by the addition of one or more cell-penetration sequences, which can include but are not limited to HIV TAT peptides, penetratin peptides, R8 peptides, transportan peptides, cyclic R8 peptides, cyclic TAT peptides, HA-TAT peptides, and xentry peptides among others. In some preferred embodiments, the cell-penetration peptide is an HIV-TAT peptide and comprises the amino acid sequence GRKKRRQRRRPQ (SEQ ID NO: 2603). In some embodiments, the inhibitor peptide is further modified with a nuclear localization sequence (NLS) which directs the peptide into the nucleus once it has crossed the plasma membrane into the cytosol of the target cell. In some embodiments, the inhibitor peptide further comprises a linker sequence between the cell-permeability sequence and the DNA-speckle motif sequence. In some embodiments, the linker comprises the amino acid sequence GGSGGGSG (SEQ ID NO: 2604). It is also contemplated that any GS-rich linker sequence known in the art may be used, and that the skilled artisan would be able to select an appropriate linker for use in the inhibitor peptides of the invention.

In some embodiments, the invention also includes a method for screening a tissue specimen in order to determine its speckle signature score. In some embodiments, the tissue specimen is cancer or tumor tissue from a subject or patient. In some embodiments, the determination of the Speckle signature score informs the use of DNA-speckle association inhibitors in order to alter the expression of speckle signature proteins in order to treat the cancer. Two speckle signatures are identified in the present disclosure, speckle Signature I and speckle signature II. The speckle signature score informs whether the gene expression pattern is primarily Signature I or Signature II. The expression of speckle Signature I correlates with poorer patient prognosis and shorter survival, and the inhibition of Signature I genes thus aids in treating the cancer. In some embodiments of the present invention, the method of determining the speckle signature score is accomplished by obtaining a specimen of tumor tissue, isolating and purifying RNA from the specimen, performing RNA-seq using the RNA to determine relative gene expression levels of speckle signature genes, and determining the Z-score of each speckle signature gene. For each speckle Signature I gene, its Z-score is divided by the number of speckle protein genes in speckle signature I, then the sum of all these values is determined for Signature I speckle protein genes. For each speckle Signature II gene, its Z-score is divided by the number of speckle protein genes in speckle signature II, then the sum of all these values is determined for Signature II speckle protein genes. Lastly, the log(2) of the ratio of the results from the previous two steps is calculated in order to determine the speckle signature score of the specimen. Samples with high positive values are strongly Signature I and samples with low negative values are strongly Signature II.

In some embodiments, the speckle signature comprises a minimal speckle signature, which comprises the genes FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, and EPC2. The minimal signature represents the smallest set of genes which can be used to separate tumor samples into Signature I or Signature II.

In some embodiments, the genes comprising speckle Signature I are selected from the group consisting of VAX2, JDP2, PLEKHN1, HDAC5, C11ORF49, SLC4A2, STYXL1, TMEM179B, TAB1, ZNF446, TBXA2R, UNC45A, PCBP1, PHLDB3, KTI12, AKAP17A, PRCC, ZNF821, SPINDOC, HSF4, DEXI, HEXIM2, EHMT2, VPS72, DDX39A, KIF22, DPCD, LHPP, CD2BP2, CDK11B, GTF2H4, DGKZ, SARNP, ALYREF, SLC2A4RG, TEPSIN, AKAP8L, PPIE, STK19, FIBP, C60RF226, H2AFX, EGFL8, PSMD13, CACTIN, EXOSC7, C120RF57, THAP4, TMEM259, THOC6, AP5Z1, PQBP1, RBM10, C1ORF35, C19ORF24, SART1, CDC34, FASTK, POMP, PRPF6, PRPF19, BRK1, UFC1, SNRPA1, ZCCHC17, SNRPB2, PCP2, SSH3, SETD1A, WDR90, THEM6, U2AF2, RBM14, MAST3, LIMK1, SF3B4, DDX39B, RTEL1, ZNF165, MAPK12, PSMD8, CDK5RAP1, PDZK1IP1, SETD4, CHTOP, CDK11A, SRSF4, TBX19, RTN2, CCDC32, CYSRT1, IQCK, MPP1, MAMSTR, ILRUN, DBNDD1, EPHB6, TCF15, C60RF52, CYGB, CCDC85C, PHYHD1, ITPKC, CDC25C, RMI2, SNRNP40, HISTIHIE, ZC3H18, SON, RBM27, TCF12, BCLAF1, ERBIN, SETD2, TCP1 IL2, EPC2, TRIP12, YLPM1, LMTK2, GPATCH8, DDX46, PRPF4B, TAB3, EPG5, RSBN1L, SF3B1, PUS7L, KCTD20, RBM26, BAZ2A, RBM41, RREB1, ZNF621, FAM160B1, CDK13, SDE2, DHX15, PRPF40A, CHIC1, SREK1, LIN52, BARD1, ZNF441, GNAQ, THRAP3, HBP1, SMC5, PPP4R3B, RBBP6, TTC26, COG6, ZC3H14, UBE3B, MRTFB, YTHDF3, UBE4A, CBLL1, API5, CMTR2, TBC1D12, WRN, KIAA1328, TMEM209, ZCCHC4, MAPK14, ZNF160, SLU7, ERCC8, FOXJ3, PCLO, RSRC1, ZC3H11A, BMP2K, RALGAPB, FBXL4, RTL6, RCAN3, FBXO34, ZBTB8A, CWF19L2, SRRM2, HELQ, FYTTD1, PPIG, ANKRD44, SOCS6, S100PBP, ZNF304, ZNF543, RBM25, EFCAB13, CPD, ARMCX5, POLI, ZNF551, MAML3, POLR3B, SFMBT2, DDX17, RNF169, KAT6A, DDX42, GPATCH2, CBFA2T2, E2F3, ZNF169, TAF5L, KIAA0100, PRKAA1, LHX4, RSRC2, CSRNP2, NCBP3, NCAPG2, SF3A1, DENND1B, BRD2, PNISR, E2F7, LRRC8B, PACSIN2, PNN, KIAA0556, SAP130, CPSF6, MAP3K7, TADA2A, HP1BP3, ZNF217, BRD1, SRRM1, SRSF11, GLYR1, FAM227B, AAGAB, PLRG1, FCHSD2, MECOM, TMEM56, CDYL, ELOA, STK17A, RIOK1, ARHGAP42, R3HCC1L, COPS4, BORCS7, THOC1, CIR1, PYROXD1, ARHGAP18, NSL1, WTAP, ZNHIT6, BCAS2, HAUS6, MORF4L1, SMC4, MBD4, PRPF18, CWC22, UBAP2L, SMURF2, KDM6B, PRKAA2, LIFR, RBM8A, SNURF, DAZAP2, FAM120C, WDR17, ZDHHC15, GTF2H2C, SRGAP1, ZSWIM5, RAF1, ZNF286B, ZNF528, ZNF572, ZNF527, XYLB, FNBP4, PRPF4, SIPA1L3, ZNF382, RFXAP, RBM39, CWC25, ZIM2, ANXA9, MFSD11, BPNT1, GPN3, MAPT, PPP1R16B, ZNF250, RAD52, ZNF786, GNB5, MNS1, TARBP1, RBM6, PRKN, ZCWPW2, MAMDC2, IPCEF1, NFATC4, LPAR1, VXN, FAM107A, IL16, USP22, RNF112, CRY2, PLAGI, IQUB, PPP1R8, BNIP3L or any combination thereof.

In some embodiments, the genes comprising speckle Signature II are selected from the group consisting of SON, RBM27, TCF12, BCLAF1, ERBIN, SETD2, TCP1 IL2, EPC2, TRIP12, YLPM1, LMTK2, GPATCH8, DDX46, PRPF4B, TAB3, EPG5, RSBN1L, SF3B1, PUS7L, KCTD20, RBM26, BAZ2A, RBM41, RREB1, ZNF621, FAM160B1, CDK13, SDE2, DHX15, PRPF40A, CHIC1, SREK1, LIN52, BARD1, ZNF441, GNAQ, THRAP3, HBP1, SMC5, PPP4R3B, RBBP6, TTC26, COG6, ZC3H14, UBE3B, MRTFB, YTHDF3, UBE4A, CBLL1, API5, CMTR2, TBC1D12, WRN, KIAA1328, TMEM209, ZCCHC4, MAPK14, ZNF160, SLU7, ERCC8, FOXJ3, PCLO, RSRC1, ZC3H11A, BMP2K, RALGAPB, FBXL4, RTL6, RCAN3, FBXO34, ZBTB8A, CWF19L2, SRRM2, HELQ, FYTTD1, PPIG, ANKRD44, SOCS6, S100PBP, ZNF304, ZNF543, RBM25, EFCAB13, CPD, ARMCX5, POLI, ZNF551, MAML3, POLR3B, SFMBT2, DDX17, RNF169, KAT6A, DDX42, GPATCH2, CBFA2T2, E2F3, ZNF169, TAF5L, KIAA0100, PRKAA1, LHX4, RSRC2, CSRNP2, NCBP3, NCAPG2, SF3A1, DENND1B, BRD2, PNISR, E2F7, LRRC8B, PACSIN2, PNN, KIAA0556, SAP130, CPSF6, MAP3K7, TADA2A, HP1BP3, ZNF217, BRD1, SRRM1, SRSF11, GLYR1, FAM227B, AAGAB, PLRG1, FCHSD2, MECOM, TMEM56, CDYL, ELOA, STK17A, RIOK1, ARHGAP42, R3HCC1L, COPS4, BORCS7, THOC1, CIR1, PYROXD1, ARHGAP18, NSL1, WTAP, ZNHIT6, BCAS2, HAUS6, MORF4L1, SMC4, MBD4, PRPF18, CWC22, UBAP2L, SMURF2, KDM6B, PRKAA2, LIFR, RBM8A, SNURF, DAZAP2, FAM120C, WDR17, ZDHHC15, GTF2H2C, SRGAP1, ZSWIM5, RAF1, ZNF286B, ZNF528, ZNF572, ZNF527, XYLB, FNBP4, PRPF4, SIPA1L3, ZNF382, RFXAP, RBM39, CWC25, ZIM2, ANXA9, MFSD11, BPNT1, GPN3, MAPT, PPP1R16B, ZNF250, RAD52, ZNF786, GNB5, MNS1, TARBP1, RBM6, PRKN, ZCWPW2, MAMDC2, IPCEF1, NFATC4, LPAR1, VXN, FAM107A, IL16, USP22, RNF112, CRY2, PLAGI, IQUB, PPP1R8, BNIP3L, VAX2, JDP2, PLEKHN1, HDAC5, C11ORF49, SLC4A2, STYXL1, TMEM179B, TAB1, ZNF446, TBXA2R, UNC45A, PCBP1, PHLDB3, KTI12, AKAP17A, PRCC, ZNF821, SPINDOC, HSF4, DEXI, HEXIM2, EHMT2, VPS72, DDX39A, KIF22, DPCD, LHPP, CD2BP2, CDK11B, GTF2H4, DGKZ, SARNP, ALYREF, SLC2A4RG, TEPSIN, AKAP8L, PPIE, STK19, FIBP, C60RF226, H2AFX, EGFL8, PSMD13, CACTIN, EXOSC7, C120RF57, THAP4, TMEM259, THOC6, AP5Z1, PQBP1, RBM10, C1ORF35, C19ORF24, SART1, CDC34, FASTK, POMP, PRPF6, PRPF19, BRK1, UFC1, SNRPA1, ZCCHC17, SNRPB2, PCP2, SSH3, SETD1A, WDR90, THEM6, U2AF2, RBM14, MAST3, LIMK1, SF3B4, DDX39B, RTEL1, ZNF165, MAPK12, PSMD8, CDK5RAP1, PDZK1IP1, SETD4, CHTOP, CDK11A, SRSF4, TBX19, RTN2, CCDC32, CYSRT1, IQCK, MPP1, MAMSTR, ILRUN, DBNDD1, EPHB6, TCF15, C60RF52, CYGB, CCDC85C, PHYHD1, ITPKC, CDC25C, RMI2, SNRNP40, HIST1H1E, ZC3H18.

In some embodiments, the invention also includes a method of treating a DNA-speckle related cancer in a subject in need thereof, comprising administering to the subject an effective amount of the pharmaceutical composition comprising the polypeptides of the invention disclosed herein, thereby treating the cancer.

In some embodiments, the invention includes a method of treating a DNA-speckle related cancer in a subject in need thereof, comprising administering to the subject an effective amount of a polypeptide comprising a first polypeptide domain, a second polypeptide domain, and a third polypeptide domain, wherein the first polypeptide domain comprises a cell penetrating peptide, the second polypeptide domain comprises a linker region, and the third polypeptide domain comprises a DNA-speckle targeting motif. In some embodiments, the DNA-speckle targeting motif comprises an amino acid sequence set forth in any one of SEQ ID NOs: 1-1730.

In some embodiments, the current disclosure also provides a method of treating a speckle signature-associated cancer in a subject in need thereof, comprising obtaining a specimen of tumor tissue, isolating and purifying RNA from the specimen, performing RNA-seq using the RNA to determine the speckle signature of the tumor tissue, and administering an effective amount of an anticancer therapeutic, thereby treating the cancer. In certain embodiments of the method, the sensitivity of the tumor to the anticancer therapeutic correlates with the speckle signature of the tumor tissue.

In certain embodiments, the speckle signature is associated with speckle signature I. In certain embodiments, the speckle signature is associated with speckle Signature II. In certain embodiments of the method, choosing a speckle signature correlated treatment strategy improves treatment prognosis. In some embodiments, the cancer is selected from the group consisting of clear cell renal cell carcinoma, neuroblastoma, KMT2D wild type melanoma, TTN wild type lung adenocarcinoma, BRAF wild type thyroid cancer, and PIK3R1 mutant endometrial cancer. In some embodiments, the anticancer therapeutic is selected from the group consisting of a biologic, a small molecule, a chemotherapeutic, an immunotherapy, and any combination thereof. It is envisioned that any anticancer treatment which can be demonstrated to have a beneficial effect which correlates with tumor speckle signature can be used with the methods of the current disclosure. In certain embodiments, the immunotherapy is an immune checkpoint inhibitor. A non-limiting example of an immune checkpoint inhibitor that demonstrates a treatment correlation with DNA speckle signature is inhibition of the PD-1 signaling pathway (e.g., by nivolumab, an anti-PD1 antibody). The PD-1 signaling pathway can be inhibited by a number of strategies, including antibody blockade of PD-1, PD-L1, PD-L2, and/or the use of receptor antagonists or non-functional ligands. Other examples of immune checkpoint inhibitors that can be used with the methods of the current disclosure include, but are not limited to inhibitors of CTLA-4, Lag-3, TIGIT, Tim-3, BTLA, VISTA, among others, including combinations thereof. In some embodiments, the therapeutic inhibitor is an inhibitor of HIF-2a. A number of HIF-2a inhibitors are known in the art, including but not limited to PT2399, PT2385, and PT2977 also known as belzutifan and MK-6482.

In some embodiments, the current disclosure provides methods for determining the speckle phenotype by measuring the localization profile of nuclear speckles within the cell nucleus in formalin-fixed, paraffin-embedded (FFPE) tumor specimens. In some embodiments, this involves at least one speckle-resident protein or other protein whose nuclear localization correlates with speckle location. Non-limiting examples of speckle-resident and/or speckle-associated proteins include, but are not limited to SON, SRRM2, and RBM25, among others. In some embodiments, the gene expression-calculated speckle signature profile corresponds to the physical location of the speckle structure within the nucleus (e.g. in the center of the nucleus or dispersed within the nucleus. For example, gene expression-calculated speckle signature II is correlated with centrally-located speckles, while gene expression-calculated speckle signature I is correlated with more dispersed speckle structures which are spread throughout the nucleus. In some embodiments, the determination of a speckle phenotype is informed by determining the expression level of one or more speckle-associated proteins. In some embodiments, the determination of a speckle phenotype is informed by determining the positioning or localization of a speckle-resident protein or a nuclear speckle structure within the nucleus. In some embodiments, the determination of a speckle phenotype is informed by both the expression level of one or more speckle-resident proteins and the positioning or localization of a speckle-associated protein or a nuclear speckle structure within the nucleus.

In some embodiments, the speckle relevant cancer displays a speckle signature. In some embodiments, the speckle signature is speckle Signature I as defined herein. In some embodiments, the expression pattern characteristic of a speckle signature correlates with worse prognosis and survival. Depending on the cancer, the speckle signature associated with worse clinical outcome can be Signature I or Signature II. In certain preferred embodiments, the cancer is clear cell renal cell carcinoma (ccRCC), wherein expression of speckle Signature I is associated with poor prognosis and survival. Because the prevalence of the speckle Signature I or speckle Signature II has been found in many types of cancer, it is contemplated that the methods of the current invention can be used in the treatment of any cancer which possesses a speckle Signature I or II gene expression pattern. Additionally, because Signature I or II gene expression patterns correspond to differential functional pathways in many different cancer types, it is contemplated that the methods of the current invention can be used to predict responses to cancer treatments in any cancer which possesses a speckle Signature I or II gene expression pattern, regardless of whether the speckle Signature gene expression pattern correlates with overall prognosis in the cancer type. Examples of cancers which have been found to express speckle signatures include but are not limited to breast cancer, cervical squamous cell carcinoma, endocervical adenocarcinoma, colon adenocarcinoma, rectum adenocarcinoma, glioblastoma, head and neck squamous cell carcinoma, kidney renal papillary cell carcinoma, glioma, liver hepatocellular carcinoma, lung squamous cell carcinoma, lung adenocarcinoma, neuroblastoma, ovarian cancer, prostate adenocarcinoma, sarcoma, skin cutaneous melanoma, stomach adenocarcinoma, tenosynovial giant cell tumor, and thymoma.

### Manipulating Nuclear Speckles by Shifting the Speckle Signature

In some embodiments, the present invention provides methods to shift gene expression programs by manipulating nuclear speckles. The applications of these methods include, but are not limited to the treatment of clear cell renal cell carcinoma, neuroblastoma, melanoma, lung adenocarcinoma, thyroid cancer, endometrial cancer, p53 gain-of-function mutant cancers, and p53 wild type cancers that are treated with p53-activating agents.

In some embodiments, the present invention provides methods to manipulate speckles from signature I-like toward signature II-like. That is, manipulations that result in decreased amounts of speckle proteins or speckle protein genes that are high in speckle Signature I and/or that result in increased amounts of speckle proteins or speckle protein genes that are high in speckle Signature II or vice versa. Methods of manipulating speckle signature can be applied to cancers and diseases where speckle signature is associated with poorer subject prognosis and/or unfavorable outcomes. The goal of such methods is to shift the DNA-speckle gene expression signature from Signature I to Signature II or vice versa, depending on which signature is associated with worse clinical outcomes. Examples of such cancers the treatment of which would benefit from speckle signature manipulation include but not limited to clear cell renal cell carcinoma, neuroblastoma, KMT2D wild type melanoma, and PIK3R1 mutant endometrial cancer, among others.

In some embodiments, the present invention provides methods to manipulate speckles from Signature II-like toward Signature I-like. That is, manipulations that result in decreased amounts of speckle proteins or speckle protein genes that are highly expressed in speckle Signature II and/or that result in increased amounts of speckle proteins or speckle protein genes that are highly expressed in speckle Signature I. Such manipulations can be applied to treat cancers and diseases where speckle Signature II is associated with poorer subject prognosis and/or unfavorable outcomes, including but not limited to TTN wild type lung adenocarcinoma and BRAF wild type thyroid cancer among others.

Methods that manipulate the nuclear speckle signature are expected to globally skew gene expression patterns. In instances where the manipulations shift from a speckle Signature I-like gene expression pattern to a speckle Signature II-like gene expression pattern, expression of speckle-associated genes are expected to be generally reduced and expression of non-speckle-associated genes are expected to be generally elevated. In instances where the manipulations shift from a speckle signature II-like signature to a speckle signature I-like signature, expression of non-speckle-associated genes are expected to be generally reduced and expression of speckle associated genes are expected to be generally elevated.

In some embodiments, inhibiting or promoting individual speckle protein genes within the speckle signature will be sufficient to shift the speckle signature. This has been demonstrated for SART1 using siRNAs to deplete SART1 levels, which indicated an interdependence of speckle protein gene expression supporting a shift in speckle signature beyond the individual target of the manipulation. Hence, any of the speckle protein genes within the speckle signature are considered to be potential therapeutic targets that may be used to shift towards a favorable speckle signature.

In some embodiments, the effectiveness of each manipulation in shifting the speckle signature is benchmarked using RNA sequencing comparing the manipulation to an appropriate control condition (i.e. non-targeting control siRNA for siRNA manipulations), assessing the degree to which the manipulation shifts gene expression patterns depending on their speckle association status, and comparing the RNA expression fold change in manipulated condition versus control to patient signature group-defined expression patterns.

In addition, shifts in the speckle signature are assessed by immunofluorescence studies of the key speckle proteins using the assays described in the present disclosure. The efficaciousness of shifting speckle signature for treating clear cell renal cell carcinoma is assessed in cell-based cancer assays, including anchorage-independent growth, invasion assays, and assessing expression properties of the cells. In addition, mouse xenograft assays can be used to determine the tumor suppressive or tumor promoting consequences of shifting the speckle signature in ccRCC pre-clinical models.

In some embodiments, the current invention includes methods for shifting the speckle signature of a particular tissue comprising the use of nucleic acid inhibitors and activators including but not limited to siRNAs, shRNAs, CRISPR/Cas9 technology, dominant negative expression plasmids, and overexpression plasmids and the like. Such inhibitory nucleic acids are well known in the art and are directed against the mRNA of one or more target genes, thereby decreasing the expression of the target genes. In some embodiments, the methods for shifting the speckle signature comprise the use of antibody inhibitors and PROTACs (proteolysis targeting chimeras) or other small molecule inhibitors that alter the amount or localization of speckle protein genes.

Measurement of Nuclear Speckle Positioning within the Nucleus

In some aspects, the current invention measures nuclear speckle positioning within the nucleus using immunofluorescence detection of the speckle-resident protein, SON, in formalin-fixed paraffin-embedded (FFPE) tissue sections. In some embodiments, the protein SON is detected using immunofluorescence microscopy using the SON antibody, ab121759 (abcam; RRID: AB_11132447). However, any antibody or specific marker that suitably labels nuclear speckles may be substituted. To assess positioning of nuclear speckles within the nucleus, the present invention makes use of a nuclear stain. In some embodiments, this nuclear stain labels DNA, such as DAPI or Hoechst 33342. In some embodiments, the nuclear speckle and nuclear stain of the current invention are detected by fluorescence microscopy. In one embodiment, images are obtained at 20× magnification on a widefield microscope (for example, Nikon Ti2E; objective: CFI60 Plan Apochromat Lambda 20× Objective Lens, N.A. 0.75, W.D. 1.0 mm, F.O.V. 25 mm, DIC, Spring Loaded), or an instrument and objective with comparable resolution. In some embodiments images are obtained at several (ie 7-9) optical sections and combined into a single maximum projection image using analysis tools typical to one familiar in the art, including, but not limited to, the MakeProjection module of the CellProfiler software. In another embodiment, images are obtained at a single in-focus optical section and used directly for subsequent calculation of nuclear speckle positioning. Nuclear speckle positioning is calculated by the fraction of nuclear speckle marker (ie SON) signal within radially-distributed bins within the cell nucleus. In one embodiment, the nucleus is fractioned radially into four bins—for example, with the first bin being the nucleus center and the fourth bin being the nucleus periphery—and the fraction of speckle signal is calculated for each bin using tools available to those familiar with the art, including, but not limited to the MeasureObjectlntensityDistribution module of CellProfiler. For each sample, per-nucleus measurements are extracted, and the median of these measurements is assigned to the subject.

As comparators, a cohort of tissue-matched tumor adjacent samples may be used. In one embodiment, high-risk ccRCC subjects are classified as those with lower speckle signal at the central nuclear fraction than the bottom 10% of the tissue-matched tumor adjacent samples. In another embodiment, high-risk ccRCC subjects are classified as those in the bottom 40% of fraction SON signal in the nucleus center of an early stage (Grade 1 and Grade 2) ccRCC cohort. It is noted that these percentages are to serve as general guidelines, and that the exact risk stratification may be contingent on the precise circumstance.

In some embodiments, other predictors of patient outcomes are paired with speckle signal radial distribution within the nucleus. These include, but are not limited to subject age, and radial distribution measurements of the DNA signal, which is also collected using the methods described in the present invention. In one embodiment, the coefficient of variation of DNA signal within the central radial fraction (ie RadialCV1of4 extracted from CellProfiler module MeasureObjectIntensityDistribution applied to DNA stained images) is used in combination with speckle radial distribution to identify high-risk subjects.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed herein.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the exemplary embodiments of the present invention, and are not to be construed as limiting in any way the remainder of the disclosure.

### Methods for Screening and Developing Peptide and Small Molecule Inhibitor Compositions

Inhibitors of speckle targeting are screened in imaging-based assays. For p53, this employed the MCF7-H2 cell line that harbors endogenously-labelled transcription sites of the p21 p53 target. MCF7-H2 cells were subjected to p53 activation with p53-activating compounds such as Nutlin-3a. Cells are then stained for immunofluorescence using the speckle marker protein, SRRM2. The cells are then imaged in well-plates, with each well containing a different speckle targeting inhibitor candidate peptide or small molecule. Known disruptors of p53-mediated speckle association, such as knockdown of the SON speckle protein gene are included on each plate as a positive control for speckle-targeting-blocking compounds. Using semi-automated image analysis software, speckle association of p21 is measured and other properties of the cells were assessed, including nuclear size, as well as speckle area and shape. For HIF2A, similar assays are performed in 786O ccRCC cell lines that have hyperactive HIF2A, and using immunoRNA-FISH for the DDIT4 HIF2A target gene. To determine transcription factor STM-targeting specificity, the concentration dependent inhibitory activities of each designed peptide are determined for each system, with the expectation that STM-containing peptides that more closely resemble the p53 STM will have higher specificity to p53-mediated speckle targeting and that peptides that more closely resemble the HIF2A STM had have higher specificity to HIF2A-mediated speckle targeting.

To assess the efficaciousness of inhibitors of speckle targeting for restricting cancer cell growth in an on-target manner, the effects of each inhibitor on proliferation are determined in cell lines that are and are not expected to be influenced by the inhibitor. For p53, this includes cancer cell lines that have gain-of-function p53 versus those that have null p53 (as in Zhu et al., 2015). For HIF2A, this includes ccRCC cell lines with hyperactive HIF2A (786O, A498, UMRC2, RCC4, and RCC10) versus primary renal tubule epithelial cell lines (i.e. HK2 or RPTEC-hTERT) and cancer cell lines without hyperactive HIF2A (i.e. cell lines used in for p53 testing). The designed compositions should lead to selective killing of p53 gain-of-function cancer cell lines (for p53-targeting STM compounds) and cancer cell lines with hyperactive HIF2A (for HIF2A-targeting STM compounds). Inhibitors of transcription factor speckle targeting are expected to have consequences on gene expression programs, reducing expression of speckle-associating transcription factor target genes and leading to either no change or an increase in non-speckle-associating transcription factor target genes. This is tested by RNA-seq and/or qRT-PCR for each successful speckle-targeting-blocking composition.

### Example 1. P53 Mediates Target Gene Association with Nuclear Speckles for Amplified RNA Expression

Recent studies have demonstrated that DNA-speckle association can be mediated by the p53 transcription factor (Alexander et al., 2021). Relevant to the present invention, it was found that not all p53 targets experience DNA-speckle association and the corresponding expression boost, and these associating and non-associating p53 targets fall into distinct functional categories. These studies also mapped the domain required for p53-mediated speckle association to the p53 proline rich domain, showing that deletion of p53 amino acids 62-77 disrupted its speckle targeting function. Likewise, mutagenesis studies of individual amino acids within p53 together with identification of a second speckle-targeting transcription factor, HIF2A (see Example 2), enabled the identification of the speckle targeting motif, derivatives of which are the basis for compositions of the present invention.

Specific Locations of Negatively Charged Amino Acids are Critical for p53-Mediated DNA Speckle Association.

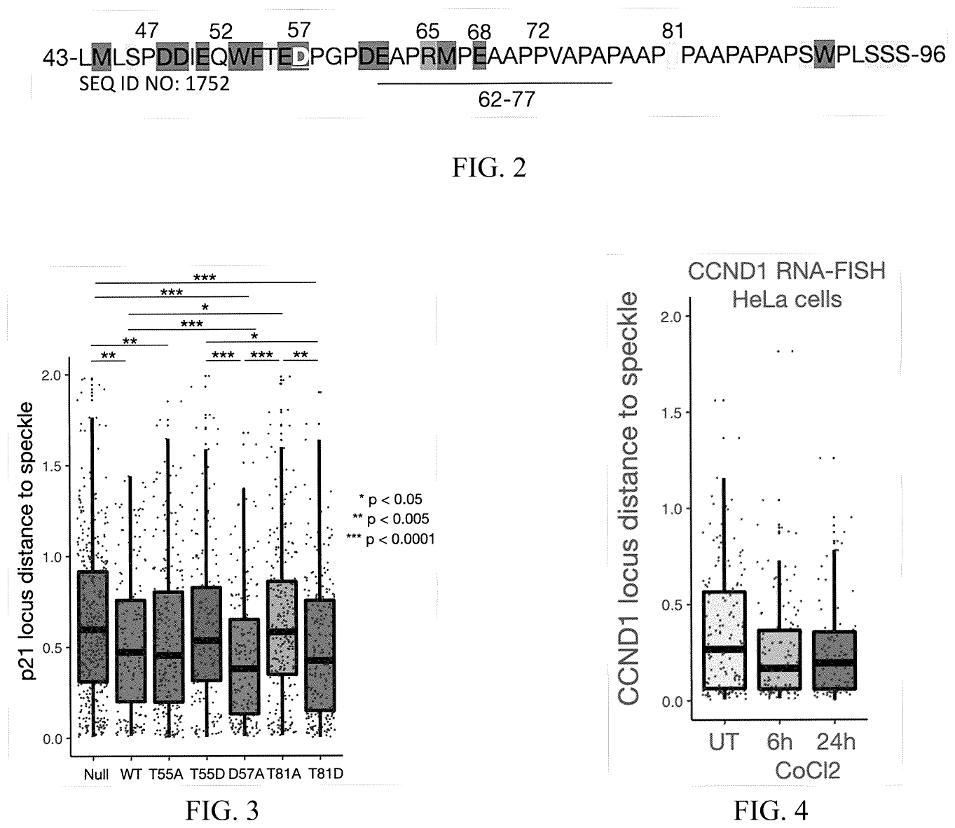

To identify the specific amino acids required for DNA-speckle association by p53, p53 point mutants were screened for speckle targeting abilities of the p21 p53 target gene using immunoDNA-FISH in the Saos2 p53-null osteosarcoma cell line induced to express exogenous wild type or mutant p53 with a doxycycline-inducible system. In these experiments, immunofluorescence with the speckle protein SON were used in combination with DNA-FISH probes to the p21 DNA locus as previously described (Alexander et al., 2021). With the expectation that previously described mutants possessing deletion of amino acids 62-77 may have disrupted those amino acids together with the chemistry of surrounding regions, the current study focused on p53 mutants spanning and surrounding this region, from P47 to T81 (P47A, D48A, D49A, Q52A, E56A, D57A, G59A, R65A, M66A, E68A, P72R, and T81A). Of these point mutations, two were identified to significantly alter the ability of p53 to drive speckle association of the p21 locus: the p53 D57A mutation, which increased p53-mediated speckle association, and the p53 T81A mutation, which decreased speckle association (FIG. 1). Of note, D57 in p53 is two amino acids away from T55, a phosphorylatable p53 residue (see FIG. 2 for p53 proline rich domain sequence). Meanwhile, T81 is also subject to regulated phosphorylation within the p53 protein. Based on the results of this mutagenesis screen, and without wishing to be bound by theory, it was hypothesized that the speckle targeting functions of p53 may be subject to regulation by phosphorylation and that p53-mediated speckle association could be manipulated by altering the negative charge at particular amino acid positions. To test this, Threonine to Alanine mutations were utilized that cannot carry a negative charge and Threonine to Aspartate mutations that are constitutively negatively charged. It was found that the p53 T55A mutant were competent at p21 speckle targeting, while the T55D mutant was defective (FIG. 3), indicating that a negative charge at the T55 residue is inhibitory towards speckle association. This finding is consistent with previous findings that elimination of negative charge in the area of D57A, improves DNA-speckle targeting by p53. Previous NMR studies of p53 phosphorylation at T55 indicate that phosphorylation of this amino acid resulted in increased contact between the second p53 transactivation domain and the p53 DNA binding domain (Sun et al., 2021). Hence, phosphorylation of T55 may obscure the proline rich domain that lies between the transactivation domain and the DNA binding domain, potentially masking it from speckle-targeting machinery.

Based on this observation, the importance of a linker region in the peptide inhibitor composition of the present invention is noted, which enables accessibility of the speckle targeting motif. Further studies and analysis, detailed below, indicate that T55 does not fall within the conserved speckle targeting motif, which instead begins at p53 amino acid 60. Thus, the effect of negative charge of T55 is more likely due to interference of other p53 protein domains with speckle targeting p53 functions.

The T81 mutation behaved in an opposite pattern to the T55 p53 mutations in that introduction of a negative charge in the T81D mutant resulted in competent p53-driven speckle association, while the uncharged T81A mutant was defective at speckle targeting (FIG. 3). Hence, the negative charge at this position supports p53 mediated DNA speckle association and is thus a critical feature for the peptide inhibitors of the present disclosure.

### Example 2. HIF2A Mediates Target Gene Association with Nuclear Speckles

Beyond p53 (Alexander et al., 2021), the extent to which other transcription factors mediate the association between specific DNA targets and nuclear speckles is not known.

Hypoxia Induction with CoCl2 Induces Speckle Association of HIF2A Target Gene CCND1.

Without wishing to be bound by theory, it was hypothesized that transcription-factor-based targeting of specific DNA sequences to speckles is a widely used mechanism of gene regulation that is employed by most eukaryotic cells. To explore this idea, speckle targeting was investigated in the context of hypoxia, a cell stress that results in the activation of hypoxia-inducible transcription factors (HIF transcription factors: HIF1A, HIF2A, and HIF3A). Using immunoRNA-FISH to measure speckle association, HeLa cells were treated with CoCl2, a mimic of hypoxia, and assessed for changes in speckle association of the HIF2A target gene CCND1. It was found that CoCl2 treatment resulted in increased speckle association of the CCND1 gene locus (FIG. 4), indicating regulated speckle association of this gene upon hypoxic stimulus, but not yet pinpointing the involvement of a specific transcription factor.

Treatment of ccRCC Cell Lines with HIF2A Inhibitor Abolishes Speckle Association.

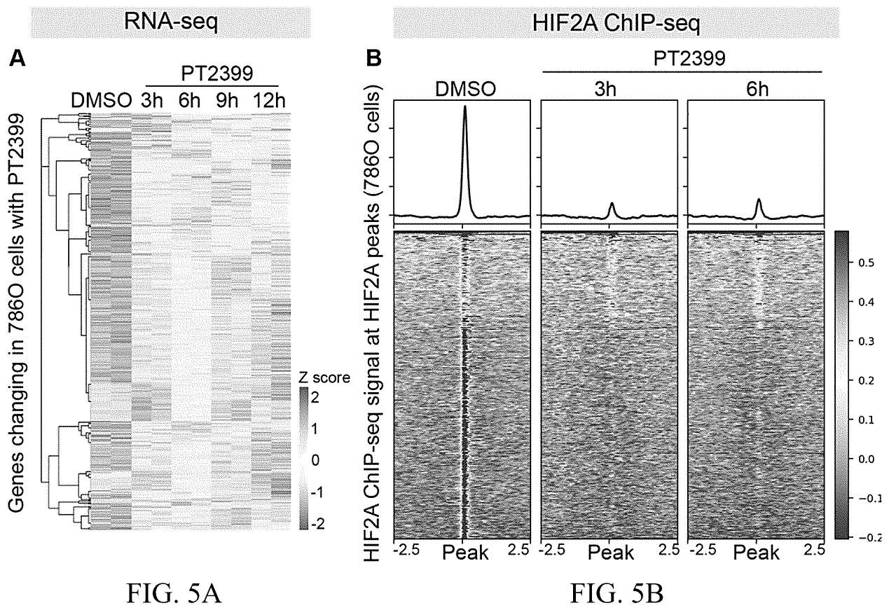

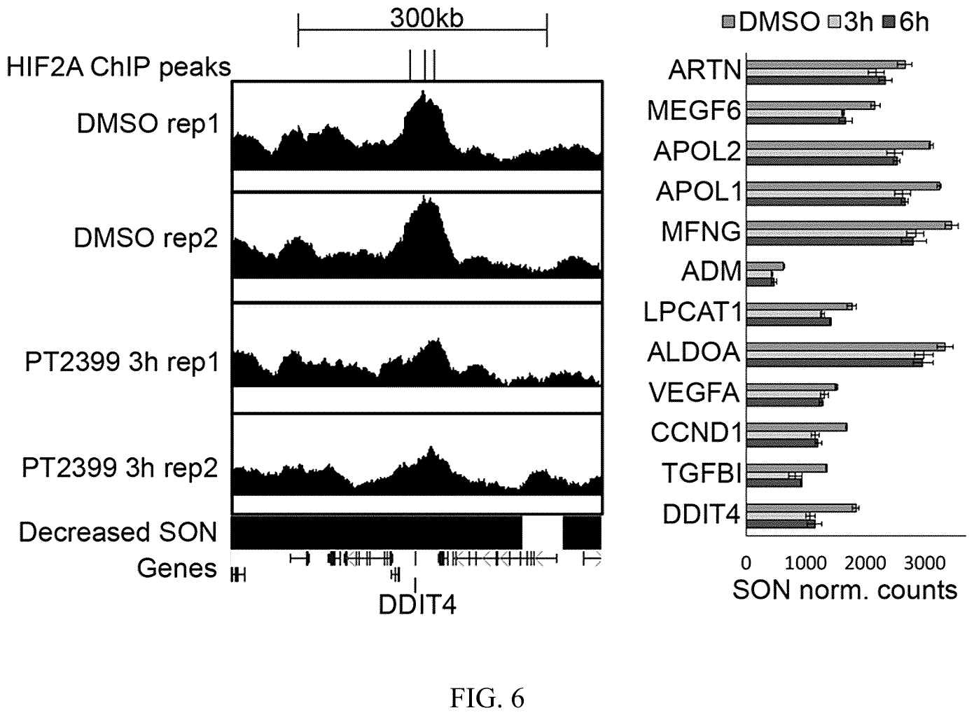

The hypoxia transcription factors are frequently hyper-active in cancer, particularly in clear cell renal cell carcinoma, which is typified by inactivating mutations in the VHL negative regulator of HIF1A and HIF2A. HIF2A inhibition as a therapeutic strategy for clear cell renal cell carcinoma has been particularly promising in pre-clinical models and in clinical trials, and a specific inhibitor targeting the interaction between HIF2A and its obligate DNA-binding heterodimer, HIF1B, has recently been FDA approved for use in individuals with germline mutations in the VHL protein. To specifically probe the role of HIF2A in maintaining speckle contacts when constitutively active in clear cell renal cell carcinoma conditions, genome-wide speckle contacts were measured using SON TSA-seq in 786O cells, a clear cell renal cell carcinoma cell line with constitutive HIF2A in the absence of HIF1A, treated with a DMSO vehicle control or with PT2399, a specific HIF2A inhibitor. To validate the on-target activity of PT2399, RNA-seq and ChIP-seq of HIF2A in 786O cells were first performed in a time-course study of PT2399 treatment (FIG. 5), which confirmed that PT2399 was behaving as expected, that is, inhibiting HIF2A genomic binding as well as HIF2A-dependent gene expression. Assessing speckle association upon PT2399 treatment identified 175 HIF2A-dependent genes (defined as genes that decrease upon PT2399 treatment) that decreased their SON TSA-seq speckle signal upon HIF2A inhibition (FIG. 6). Like p53-mediated speckle association, the speckle-associating HIF2A targets were of distinct functional categories as compared to the non-associating HIF2A targets. These studies establish HIF2A as a second transcription factor capable of driving DNA-speckle association of gene targets and provide additional evidence that speckle-associating abilities of transcription factors may benefit particular classes of target genes. This aspect is of particular importance to the present disclosure, in those changes in speckle association or in speckle content are capable of shifting the type of gene expression programs within cells.

HIF2A has a Homologous Domain to p53, Identifying it as a Conserved Speckle Targeting Motif.

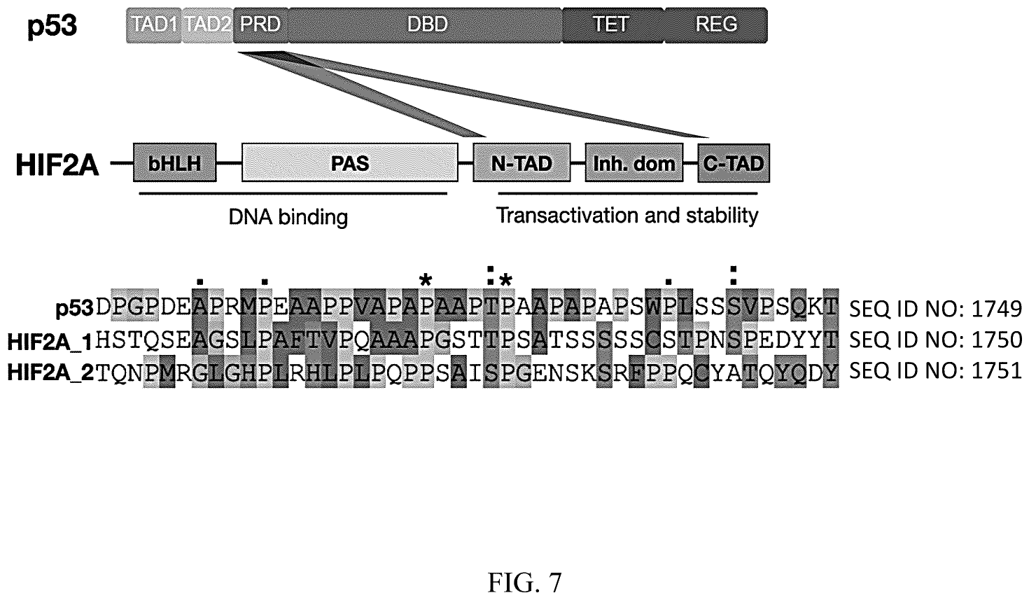

The identification of a second speckle-targeting transcription factor allowed the comparison of the two factors in search for a homologous motif that confers speckle-targeting abilities. To do so, a pairwise alignment tool that searches for local peptide sequence similarities was utilized (EMBOSS Matcher). This tool found that the most similar amino acid sequence between p53 and HIF2A was p53 amino acids 62-90 with HIF2A amino acids 450-478 (FIG. 7). This finding matched exactly with previous experiments showing that p53 amino acids 62-77 were essential for p53-mediated speckle association and provided additional insight into the finding that the charge status of p53 amino acid T81 modulates p53-speckle targeting of p21. Based on our combined observations of the centrality of the p53 T81 amino acid to this conserved motif, termed the speckle targeting motif, together with our finding that the T81A mutation abolishes p53-mediated speckle association, we posit that this particular Threonine is a key feature of the speckle targeting motif. The other key similarity between the HIF2A and p53 speckle targeting motifs is the periodicity of Proline amino acids, which occur every five amino acids in the HIF2A and p53 speckle targeting motifs leading up to the central TP/SP dipeptide and continue at this periodicity for the p53 speckle targeting motif.

A Search of the Proteome Reveals that the Speckle Targeting Motif is a Recurring Structure Found in Regulators of Gene Expression.

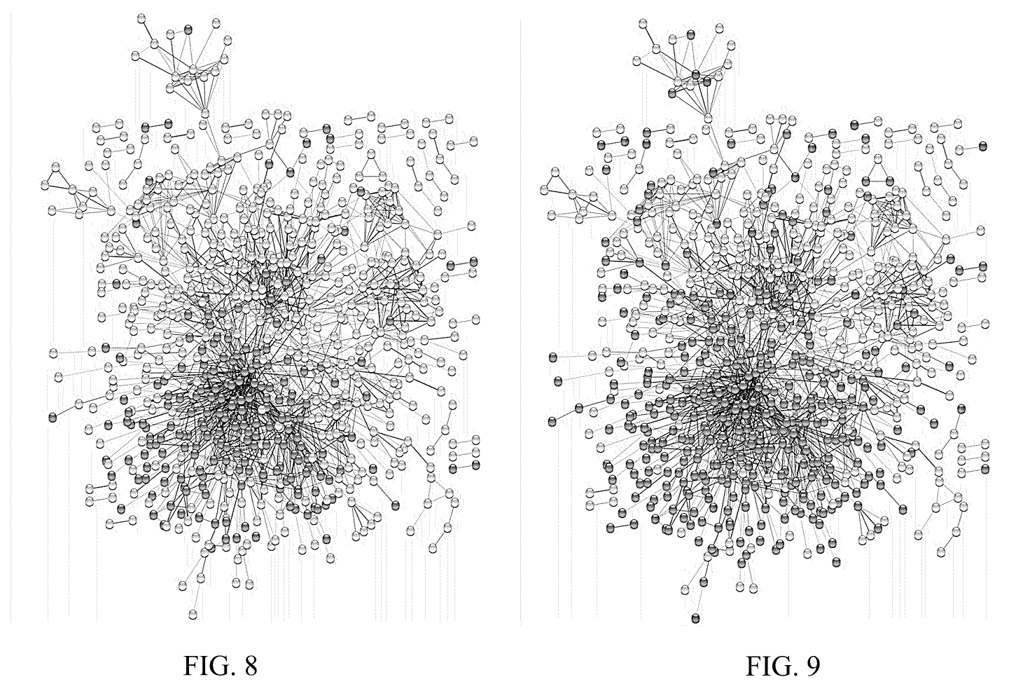

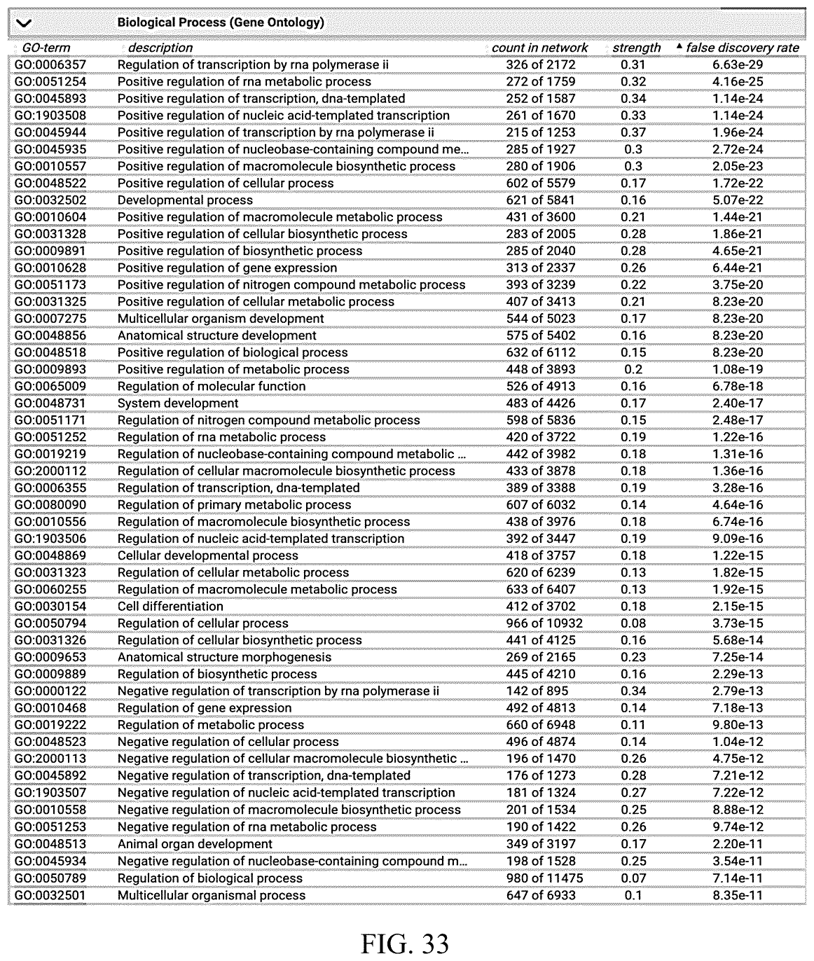

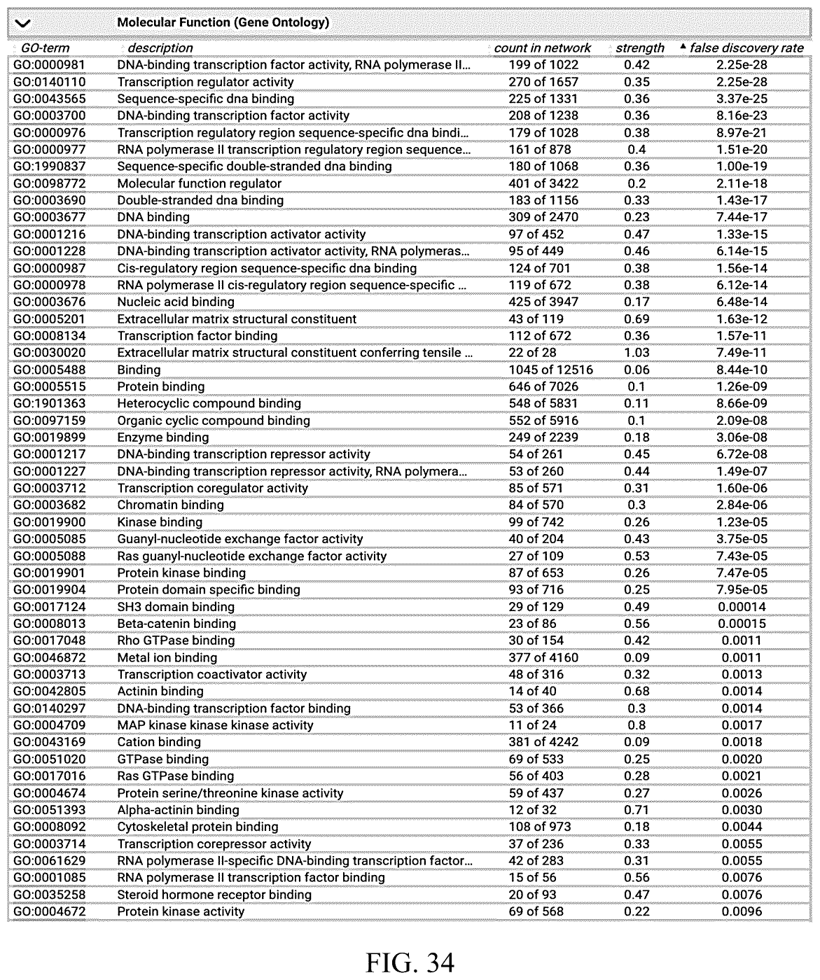

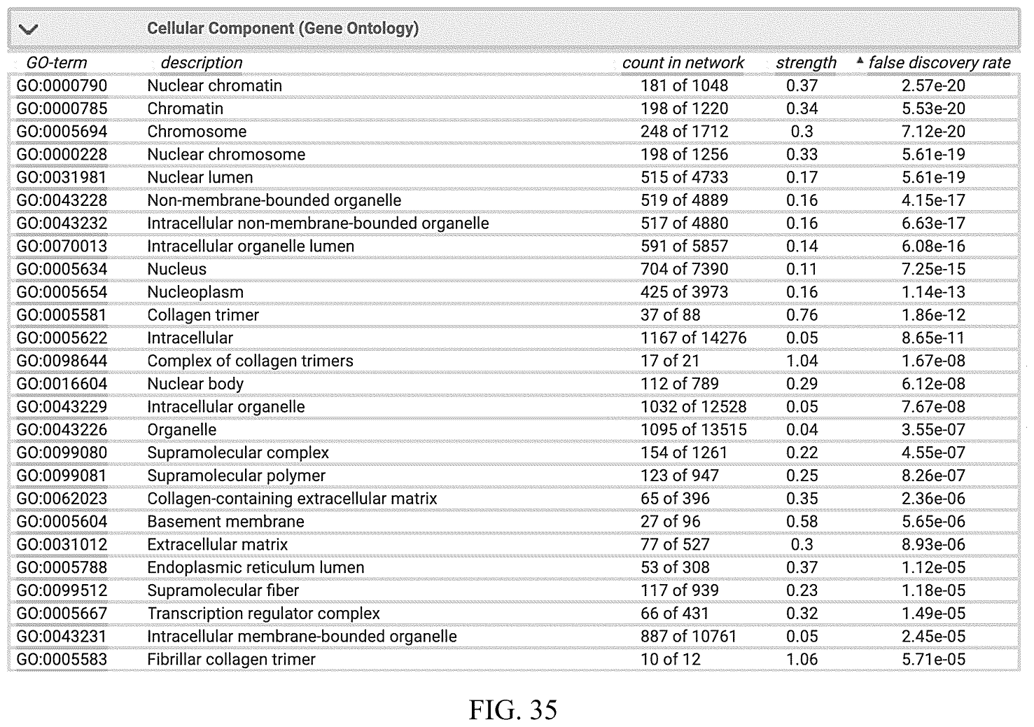

A search of the proteome revealed that the speckle targeting motif is a recurring structure found in regulators of gene expression. Based on the discovery of a conserved speckle targeting motif between HIF2A and p53, a set of properties was devised for speckle targeting motifs in general. Based on this definition, studies then used the MOTIF2 online tool to extract all human peptides with the x(30)-[TSED]-P-x(30) or x(30)-[TS]-P-x(30) motifs in separate analyses. A Python program was then written to format the files and apply the aforementioned properties. This approach identified 1075 proteins (for x(30)-[TS]-P-x(30); Table 1) and 1460 proteins (for x(30)-[TSED]-P-x(30); Table 2) that harbored putative speckle targeting motifs. Inputting these proteins into STRING-DB, a database of protein-protein interactions, it was found that speckle target motif-containing proteins were more likely to be interconnected with one another compared with random chance in a physical protein interaction network (p<1⁻¹⁶; FIGS. 8 and 9 show connected components of the network). The speckle target motif-containing proteins were extremely enriched in Biological Process, Molecular Function, and Cellular Component categories relating to RNA production and nuclear chromatin (see FIG. 33 for Biological Process; FIG. 34 for Molecular Function; FIG. 35 for Cellular Component). These discoveries revealed that the speckle targeting motif recurs among proteins involved in gene expression and that are found within the cell nucleus, specifically among factors that bind DNA. For the disclosure of the present invention, these observations provide support for the broad utility of compositions and methods that target DNA-speckle association by gene regulators and that target nuclear speckles. In parallel, the identification of biologically occurring speckle targeting motifs helps guide decisions for manipulation of the biochemical properties of the compositions of the present invention.

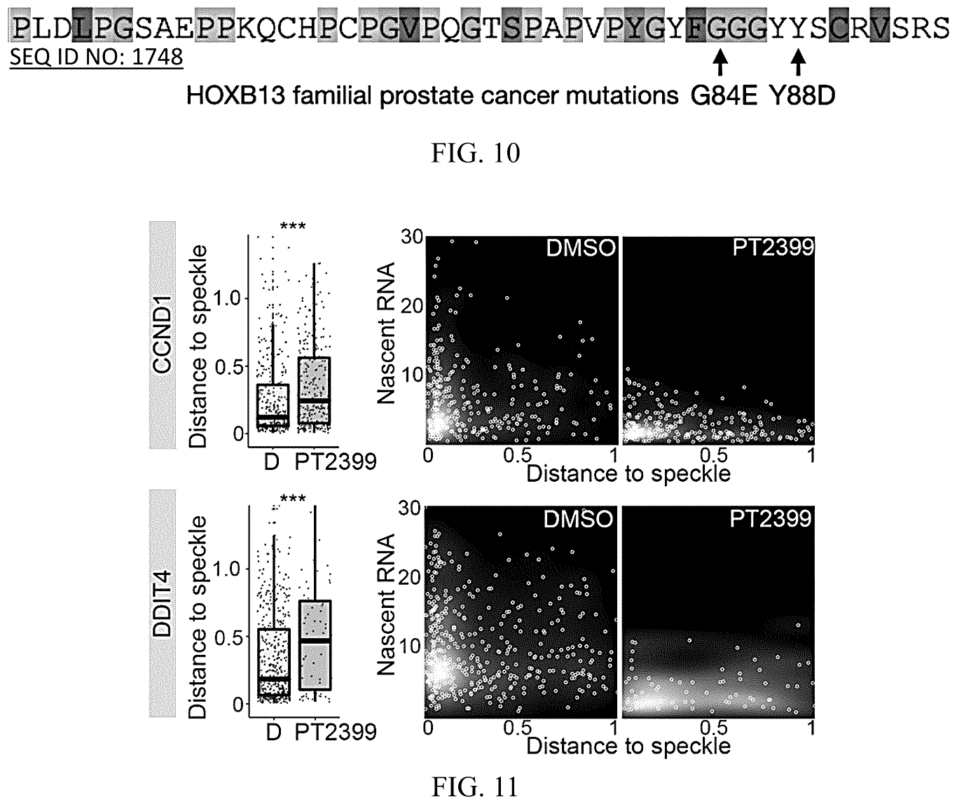

Proteins that contain speckle targeting motifs include many factors that are of high interest for therapeutic targeting. Of particular interest for commercial development are:
1. KLF4, OCT4, and TOX4 in the context of induced pluripotent stem cell generation
2. Factors implicated in T cell function and T cell exhaustion, including FLIT, TOX2, and HIVEP3.
3. Factors involved in neurogenesis (including NEUROD1), mental health (which was enriched within the disease category of factors with speckle targeting motifs; FIG. 9), and neurodegeneration (including HTT, the protein responsible for Huntington disease).
4. HOXB13, which contains genetic risk factor for prostate cancer within speckle targeting motif (FIG. 10).

### Example 3. Nuclear Speckles Broadly Regulate Gene Expression in Clear Cell Renal Cell Carcinoma and are Predictive of Patient Outcomes

Here the present disclosure demonstrates that nuclear speckle expression patterns are predictive of patient survival in ccRCC and can be manipulated to globally shift gene expression patterns depending on gene speckle association status.

ccRCC Cell Lines Differ in Speckle Association Phenotypes and Functions.

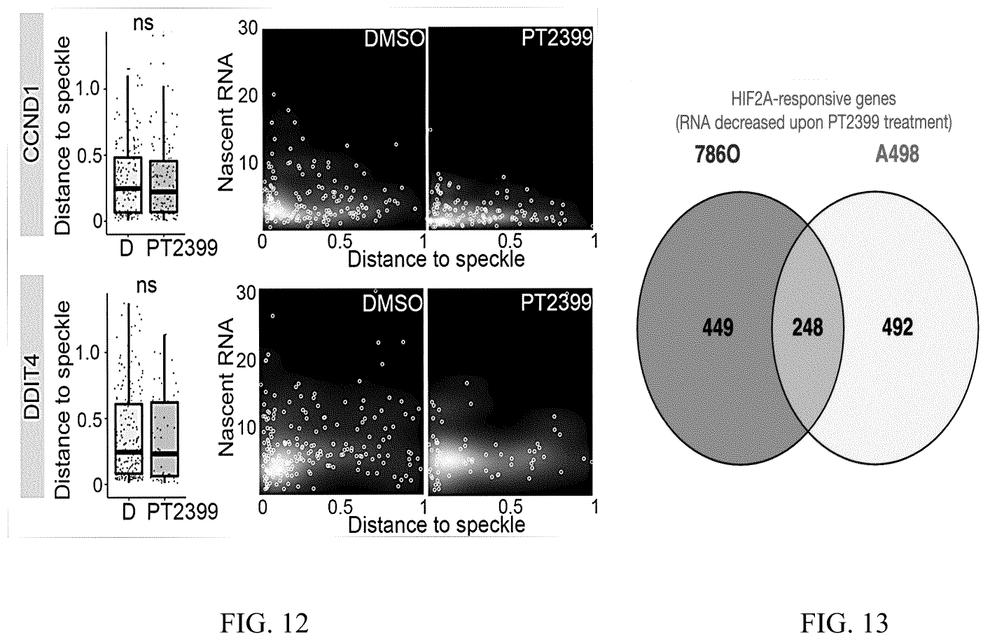

As an independent method to validate the speckle targeting activities of HIF2A in clear cell renal cell carcinoma observed in Example 2, immunoRNA-FISH experiments were used to measure changes in speckle association upon HIF2A inhibition with the PT2399 drug. These experiments used 786O cells, which were used for the genomics experiments in Example 2, and A498 cells, another ccRCC cell line that, like 786O cells, have hyperactive HIF2A in the absence of HIF1A. Consistent with our SON TSA-seq experiments, 786O cells showed HIF2A-dependent speckle association of HIF2A-responsive genes CCND1 and DDIT4 (FIG. 11). Under control, HIF2A hyperactive conditions, these cells displayed an L-shaped relationship between nascent RNA amount within transcription sites and distance to speckle, indicating that speckle-adjacent transcription sites accumulate nascent RNAs. These findings were similar to previously published observations of RNA-FISH with p53-mediated speckle association. In contrast, A498 cells did not show HIF2A-dependent changes in gene-speckle association or the L-shaped relationship between nascent RNA amounts and distance to speckle (FIG. 12). Thus, these two different ccRCC cell lines differ in their speckle association phenotypes. PT2399 treatment of each cell type resulted in a comparable number of decreased genes in each cell type (FIG. 13), indicating that this cell type difference was not due to different degrees of responsiveness to the HIF2A inhibitor. Although there were many HIF2A-responsive genes that were uniquely regulated in one or the other cell line. Hence, 786O cells and A498 cells differ both in speckle-association phenotypes as well as which genes are responsive to HIF2A inhibition.

Nuclear Speckle Content Varies Among ccRCC Patients.

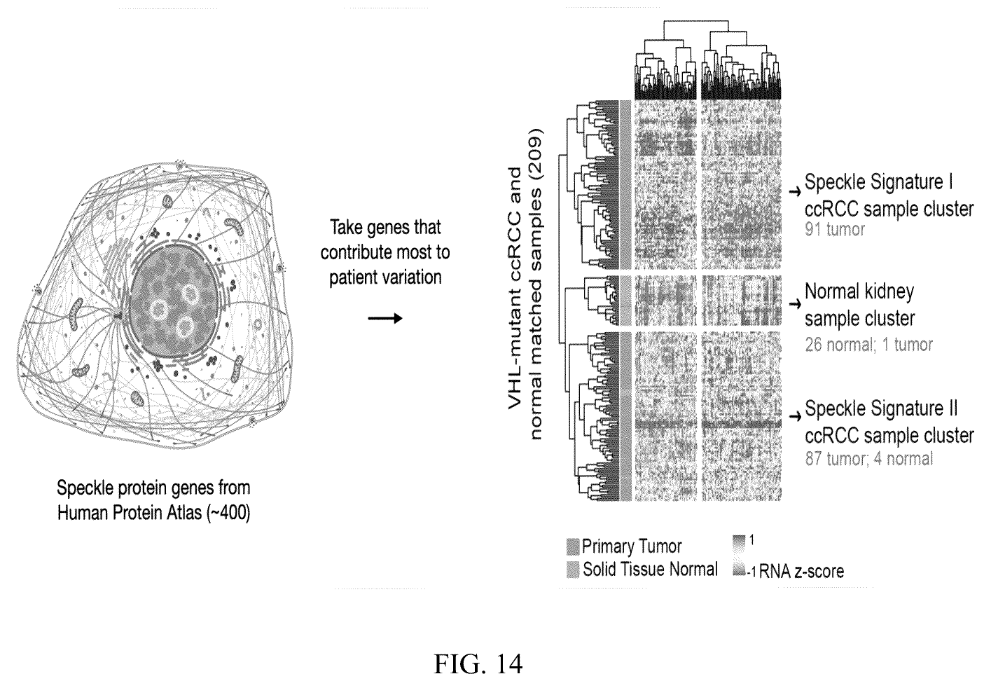

Given the present findings of cell type variations in speckle association phenotypes between the two patient-derived ccRCC cell lines, the existence of patient-to-patient variation in nuclear speckles was then investigated. To examine this, the Human Protein Atlas was used to extract speckle-resident proteins and their RNA expression was determined using The Cancer Genome Atlas (TCGA) RNA-seq data downloaded from the GDC in September 2021. To focus on HIF2A-driven clear cell renal cell carcinoma, this analysis specifically used patient tumor samples and tissue-adjacent controls from the subset of VHL-mutated patients among the kirc TCGA cohort. To narrow upon the most differential speckle protein genes, the genes that contributed most to patient variation were extracted in principle component analysis principal component 1 (PC1). Hierarchical clustering of expression of these speckle protein genes showed that tissues separated into three distinct speckle protein gene expression clusters: two tumor clusters (called Signature I and II) and a normal tissue cluster (FIG. 14). Both tumor clusters show aberrant expression of speckle protein genes as compared to the normal tissue cluster. However, the speckle Signature I patient cluster is more dissimilar to normal tissue and displays reciprocal expression of speckle protein genes compared to the speckle Signature II patient cluster. These results demonstrate that ccRCC patients can be split into two groups based on their speckle protein gene expression patterns.

Speckle Signature I is Associated with Poor Patient Outcomes and Molecular Features.

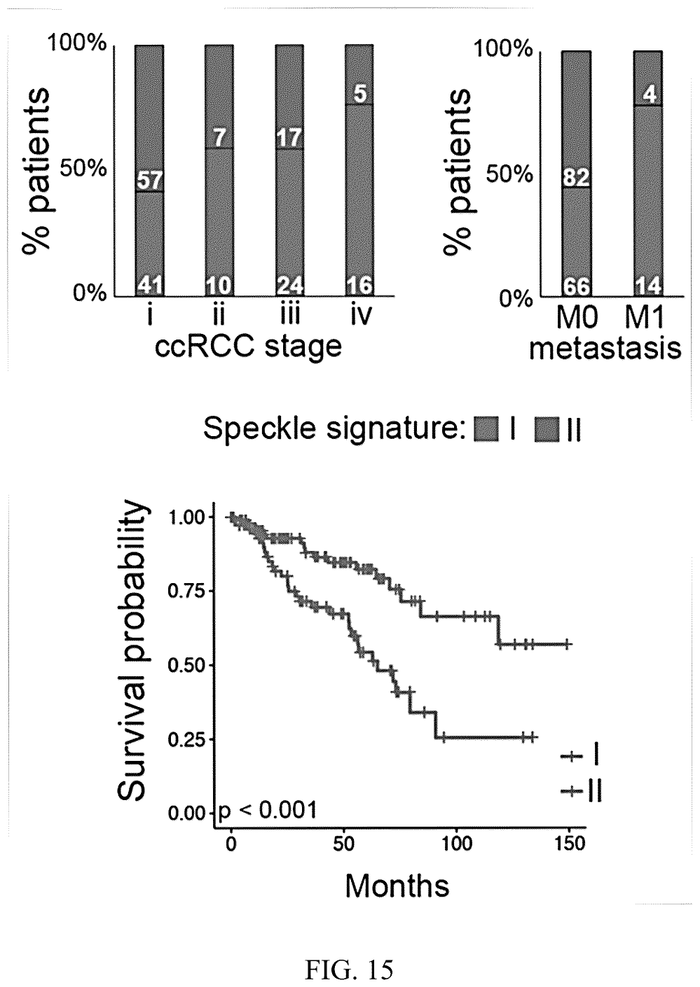

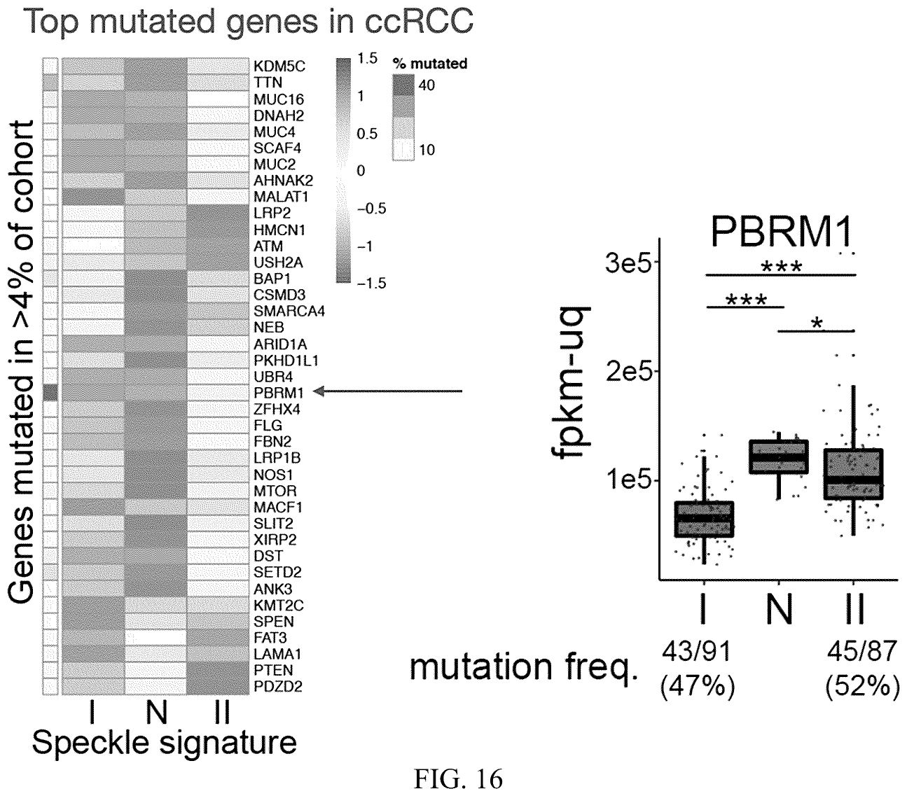

To illuminate whether speckle signature may impact patient outcomes, studies next compared clinical characteristics of patients with speckle Signature I versus speckle Signature II (FIG. 15). It was found that patients with speckle Signature I were more likely to have advanced stages of ccRCC, were more likely to have metastatic disease, and had significantly poorer overall survival compared to patients with speckle signature II. To understand the etiology of the poor outcomes in the speckle Signature I patient group, we assessed expression patterns of the top mutated genes in ccRCC within the patient cohort. While mutation frequencies did not differ between patient groups, the expression of the top mutated genes in ccRCC did differ (FIG. 16). For example, the only gene mutated in the VHL-mutant ccRCC cohort in more than 10% of patients was PBRM1. PBRM1 was mutated in a similar percentage of tumors with speckle Signature I and Signature II, but notably was expressed at lower levels in tumors with speckle signature I. Thus, the speckle signature may be an alternative strategy used by tumors to drive decreased or increased function of particular cancer-critical genes. These findings highlight the finding that separating patients by speckle signature provides a new means by which to sub classify ccRCC patients that differ their prognosis and in key molecular features of ccRCC.

### Biased Expression of HIF2A-Responsive Genes Between the Speckle Signature Patient Groups.

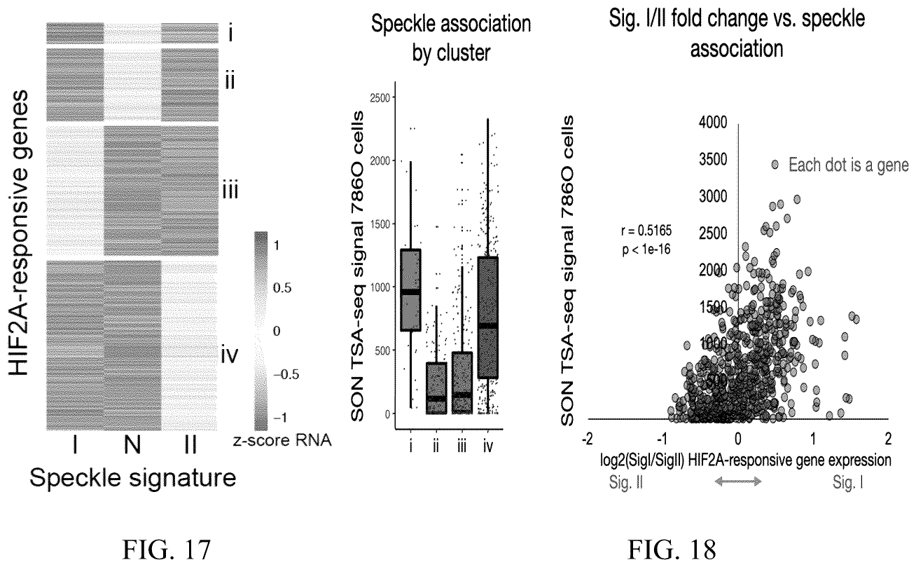

Studies next investigated whether the speckle signature alters expression of HIF2A-responsive genes. Separating the patients by speckle signature, it was found that certain HIF2A-responsive genes were preferentially expressed in samples with speckle signature I, while others were preferentially expressed in samples with speckle Signature II (FIG. 17). The HIF2A-responsive genes preferentially induced within Signature I versus Signature II patients belonged to distinct functional categories, indicating that the HIF2A functional program differs between these two patient groups. These data provide further evidence that the speckle protein gene expression signature defines distinct subclasses of ccRCC.

Expression Biases Between the Speckle Signature Patient Groups is Highly Correlated with Gene Speckle Association Status.

The findings of the present disclosure link a nuclear speckle phenotype to patient outcomes and indicate that nuclear speckles and DNA-speckle association are consequential and widespread gene regulatory mechanisms that shift transcription factor functional programs. As such, it can be hypothesized that speckle signature in ccRCC shifts expression of genes depending on their speckle association status. The speckle association status of HIF2A-responsive genes was first examined based on whether they were preferentially expressed in the Signature I or Signature II ccRCC patient groups. This analysis revealed that Signature I-biased HIF2A-responsive genes have high amounts of speckle association, while Signature II-biased HIF2A-responsive genes have low amounts of speckle association (FIG. 18). In a quantitative analysis taking the ratio of gene expression in the Signature I to II patient group versus the SON TSA-seq speckle association signal, there was a highly significant correlation (Linear Regression p<1⁻¹⁶) indicating that speckle associating genes are much more likely to be highly expressed in the Signature I patient group, while non-speckle associating genes are much more likely to be highly expressed in the Signature II patient group. These data demonstrate a strong link between speckle phenotype and expression of speckle-associating genes and also suggest reciprocal regulation of speckle and non-speckle-associating genes predicted by the speckle signature.

### 786O Cells More Closely Resemble the Speckle Signature I Patient Group.

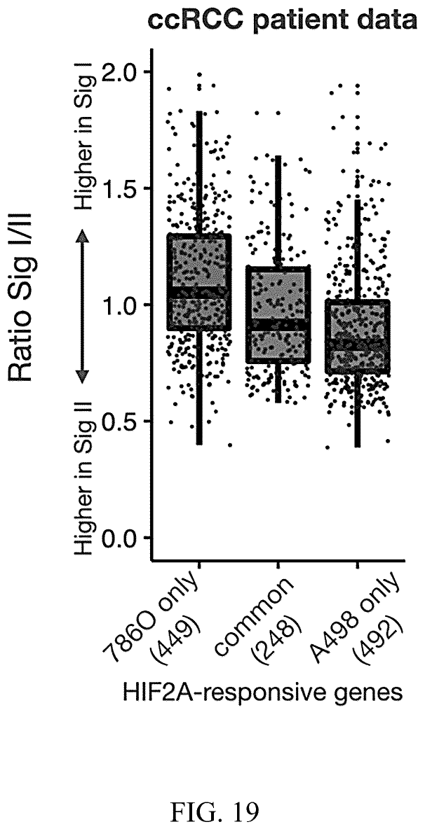

The determination of speckle signatures in ccRCC patients disclosed herein provides additional context to understand previous findings of differences between the 786O cell line, where HIF2A was required for speckle association and HIF2A targets displayed a speckle-association boost in nascent RNA (FIG. 11), and the A498 cell line where HIF2A did not regulate speckle association and did not display speckle-associated boosts in nascent RNA (FIG. 12). To investigate whether 786O and A498 cells reflected the different speckle signature patient groups, it was then assessed whether the 786O-specific and A498-specific HIF2A target genes previously identified in RNA-seq studies (FIG. 13) showed biased expression in the patient speckle signature groups. This analysis revealed that 786O-specific HIF2A-responsive genes were biased toward being highly expressed in the speckle Signature I patient group, the group of genes that was commonly regulated in both 786O and A498 cells showed little bias between patient groups, and the group of A498-specific HIF2A-responsive genes was biased toward being highly expressed in the speckle Signature II patient group (FIG. 19; p-value for each comparison <1⁻¹⁶). Hence, 786O cells more closely resemble the speckle Signature I patient group, which is biased towards higher expression of speckle associating genes. This finding is consistent with previous findings of the relationship between speckle association and boosted amounts of nascent RNA in 786O cells, but not A498 cells (compare FIGS. 11 and 12).

### Depletion of Speckle Signature I Speckle Protein Gene, SART1, Compromises Expression of Speckle Associated Genes and Boosts Expression of Non-Speckle Associating Genes in 786O Cells.

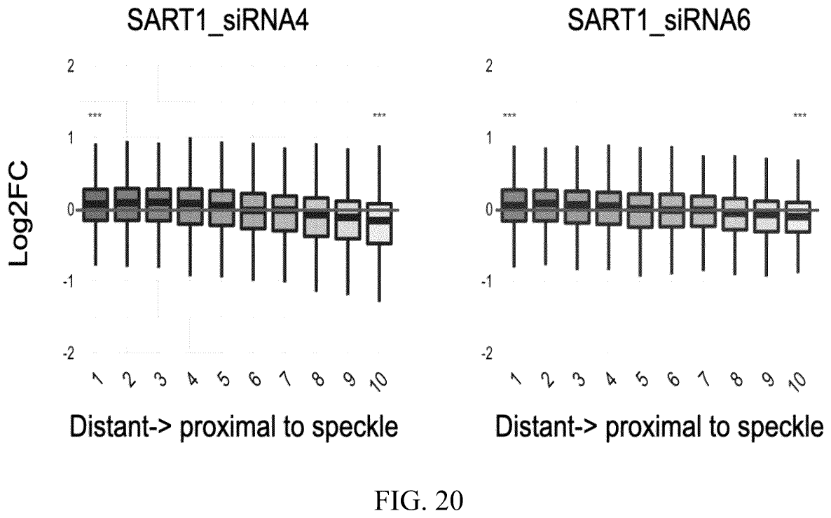

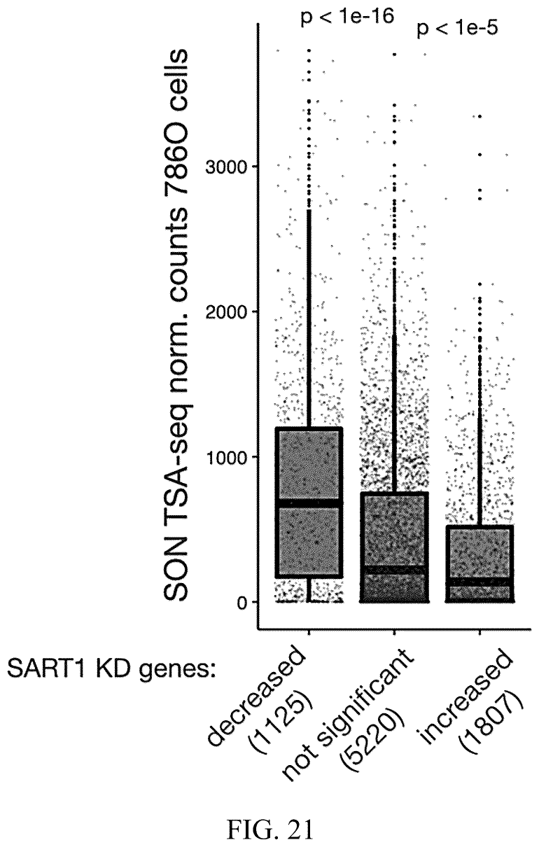

The present findings suggest that speckle Signature I supports expression of speckle associating genes and worsens patient outcomes in ccRCC, while speckle Signature II supports expression of non-speckle-associating genes and improves patient outcomes. To functionally test this, studies next sought to shift the Signature I-like 786O cells toward a Signature II-like phenotype by manipulating the expression levels of speckle protein genes. When compared to A498 cells, 786O cells have significantly higher expression levels of 27 of the speckle protein genes that are high in speckle signature I. As a proof-of-principle experiment, one of these Signature I speckle protein genes, SART1, was selected and knocked-down in 786O cells. Splitting the genome up into deciles based on gene speckle association levels, and graphing the fold change of gene expression upon SART1 siRNA knockdown, it was found that SART1 knockdown resulted in a global decrease in expression of speckle-associated genes (FIG. 20; Group 10) together with a global increase in expression of non-speckle associated genes (FIG. 20; Group 1), supporting the conclusion that speckle Signature I promotes expression of speckle-associating genes. In a second analysis, genes decreasing upon SART1 knockdown were found to have higher speckle association than unchanged genes, and that genes increasing upon SART1 knockdown have lower speckle association than unchanged genes (FIG. 21). Together, these data provide strong evidence that SART1 depletion shifts gene expression away from speckle associated genes in favor of non-speckle-associated genes. It also supports the concept of reciprocal expression of speckle associating and non-speckle-associating genes.

### Depletion of Speckle Signature I Speckle Protein Gene, SART1, Transforms 786O Cells Toward a Speckle Signature II-Like Expression Phenotype.

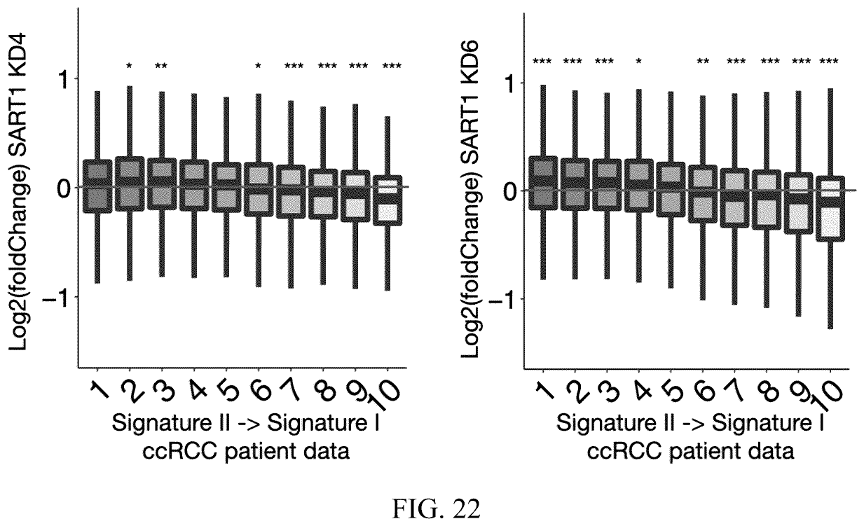

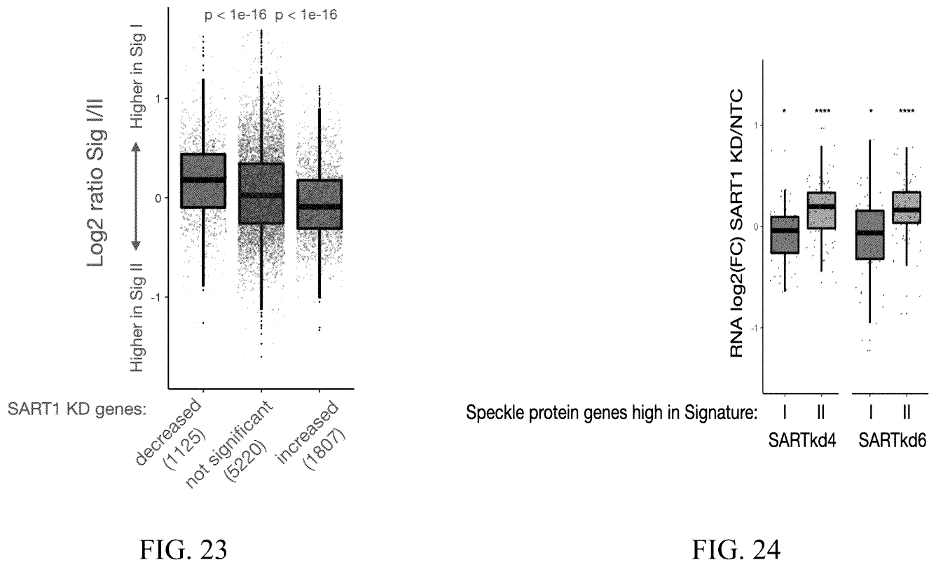

Studies next investigated whether SART1 siRNA knockdown altered the expression patterns of Signature I and Signature II biased genes. To accomplish this, the genome was split up into deciles based on gene expression bias to Signature I versus Signature II, and the fold change upon SART1 knockdown was examined within each bin. The Signature I-biased genes were found to be significantly decreased upon SART1 knockdown (FIG. 22, Groups 6-10), while the Signature II-biased genes were significantly increased upon SART1 knockdown (FIG. 22; Groups 1-4). Using a separate analysis to demonstrate the same principle, genes whose expression decreases upon SART1 knockdown are biased to the speckle Signature I patient group, while genes not changing upon SART1 knockdown are not biased toward either patient group, and genes increasing upon SART1 knockdown are biased toward the speckle Signature II patient group (FIG. 23). Together, these results demonstrate that knockdown of an individual speckle protein gene is capable of driving global shifts in gene expression that transform 786O cells from a Signature I expression phenotype toward a Signature II expression phenotype.

Because the speckle signature involves expression patterns of ˜100 speckle protein genes, it was somewhat unexpected that the knockdown of a single speckle protein gene was sufficient to shift cells from a Signature I to a Signature II expression phenotype. To explore how a single gene knockdown is capable of driving this transformation, the consequences of SART1 knockdown on the expression of other speckle protein genes was investigated. This analysis revealed that SART1 knockdown results in a modest, but significant, decrease in expression of the other speckle Signature I speckle protein genes together with a robust increase in the expression of Signature II speckle protein genes (FIG. 24). These results suggest that the presence of an interconnected speckle regulatory circuit that is capable of toggling between speckle signatures. The presence of such regulatory feedback on the speckle signature helps explain observations that tumor samples segregate into two reciprocally-expressed speckle groups, with few to no patient cases showing globally high expression or globally low expression of all identified speckle protein genes.

### Other Regulators of Speckle Signature.

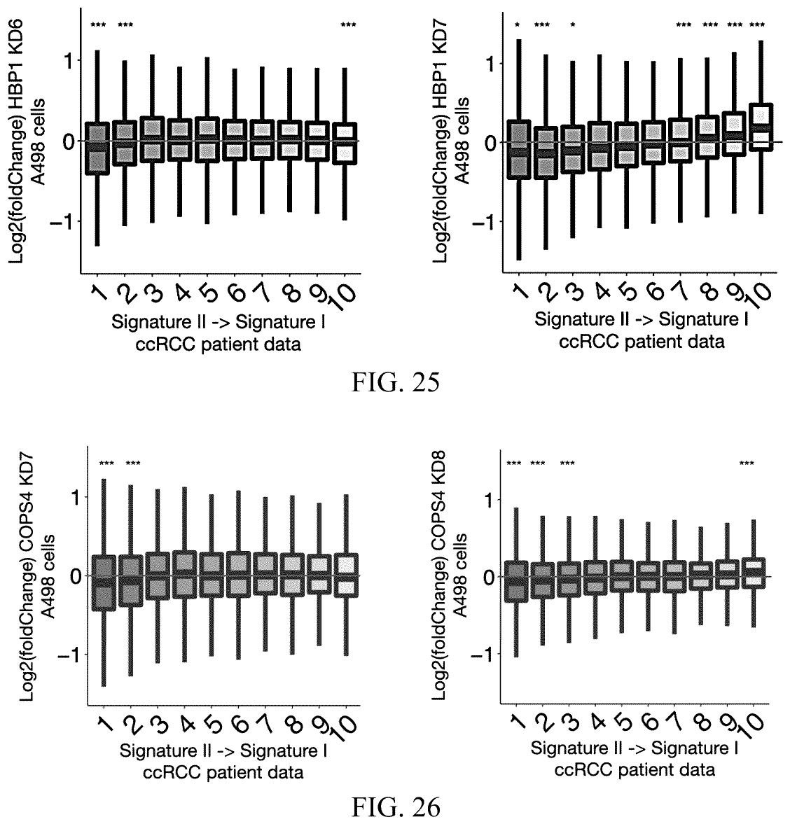

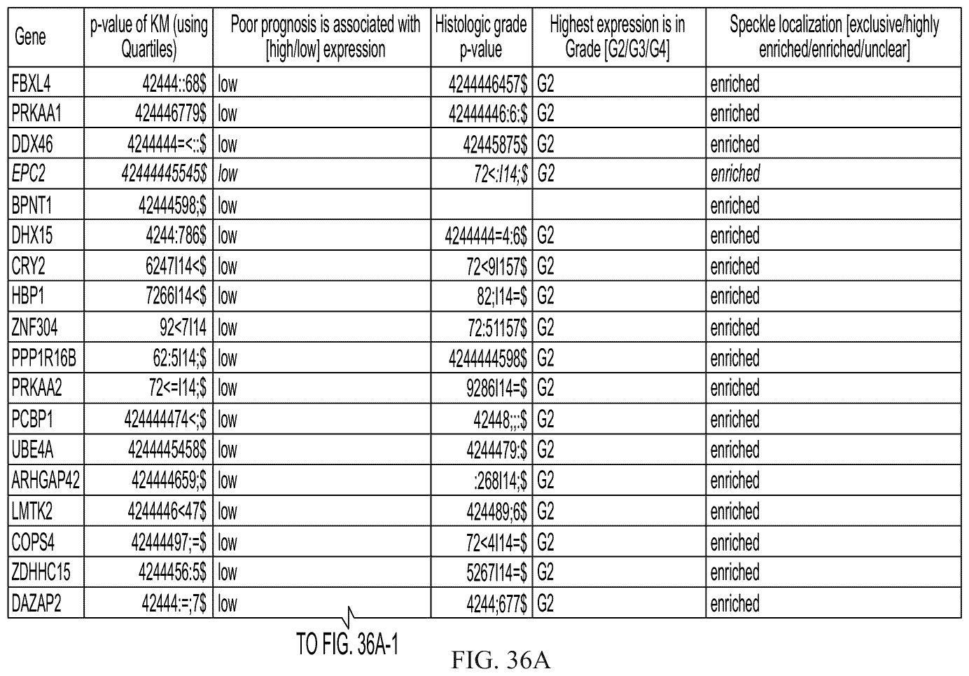

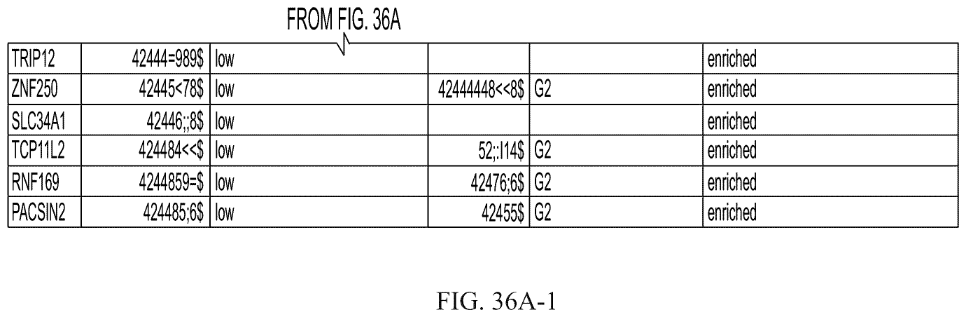

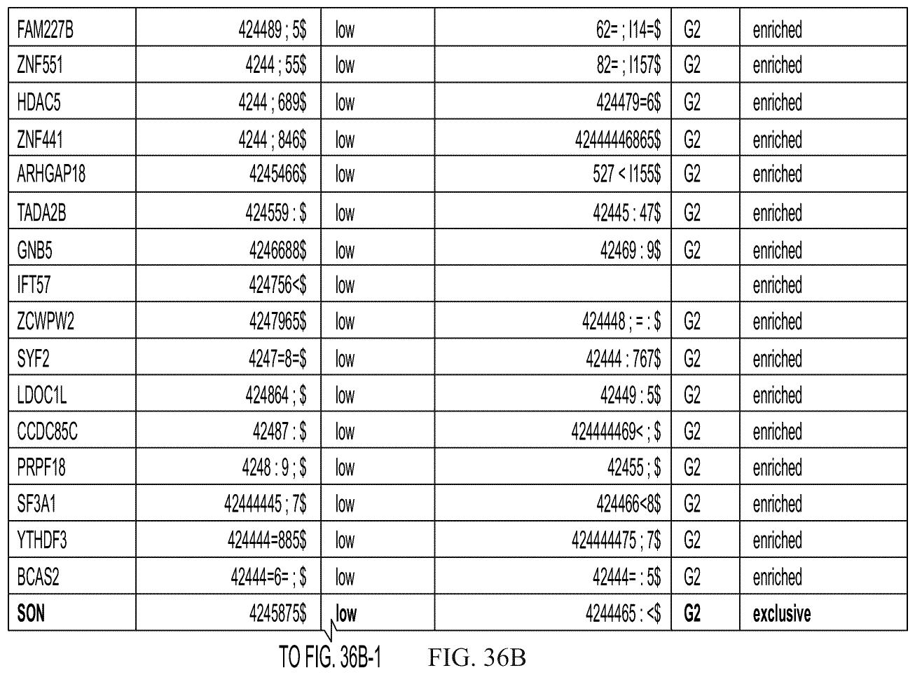

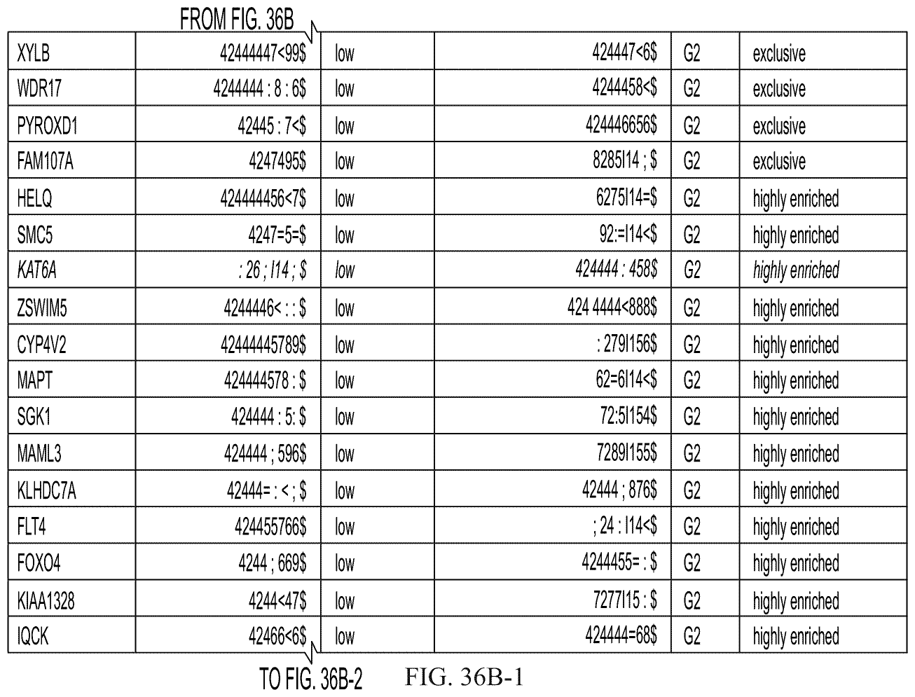

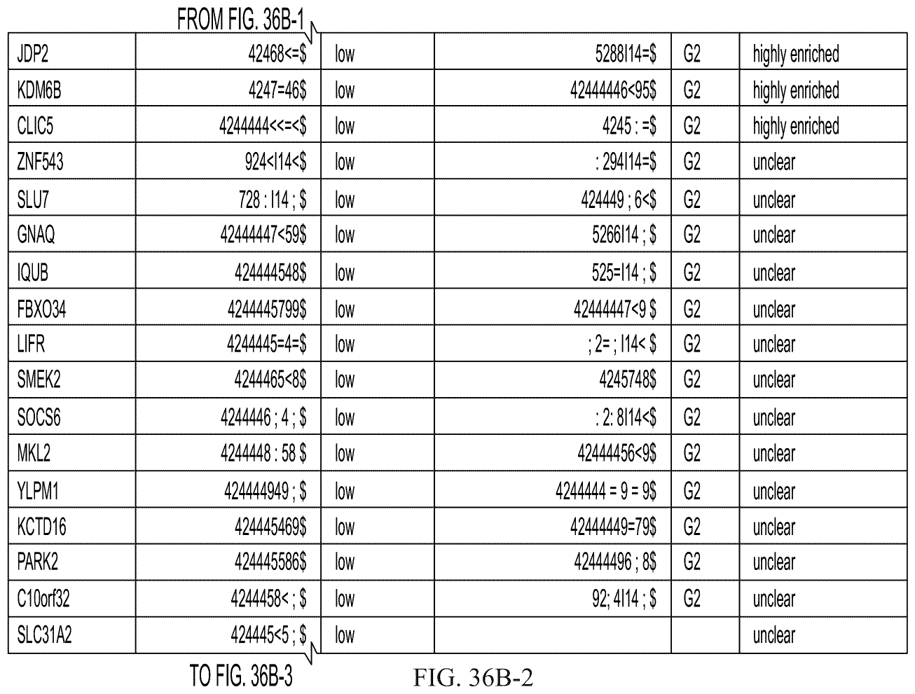

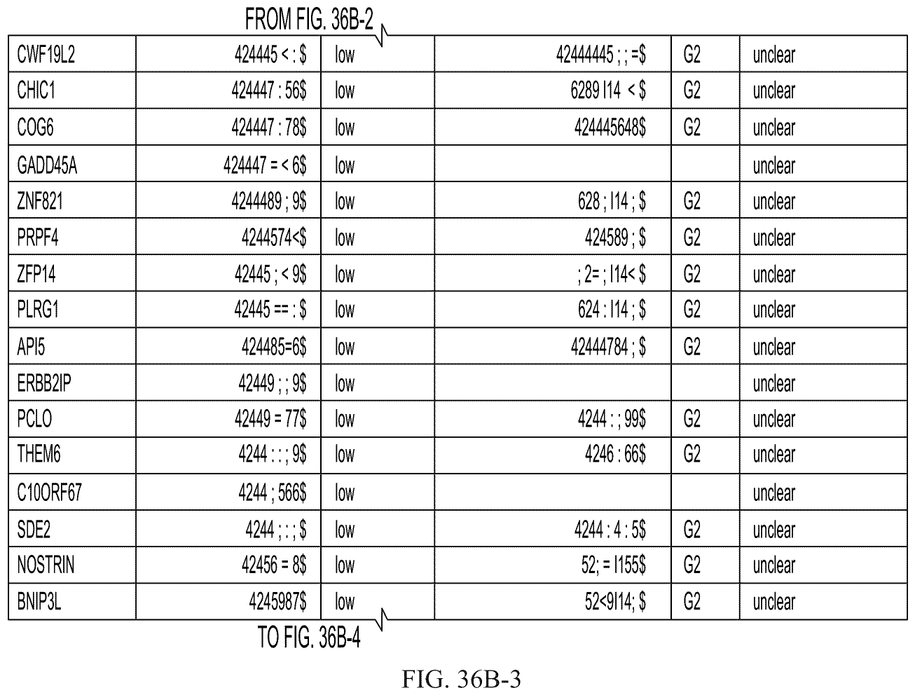

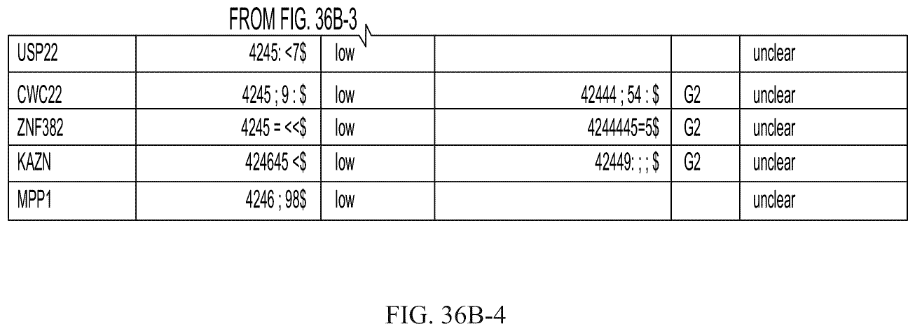

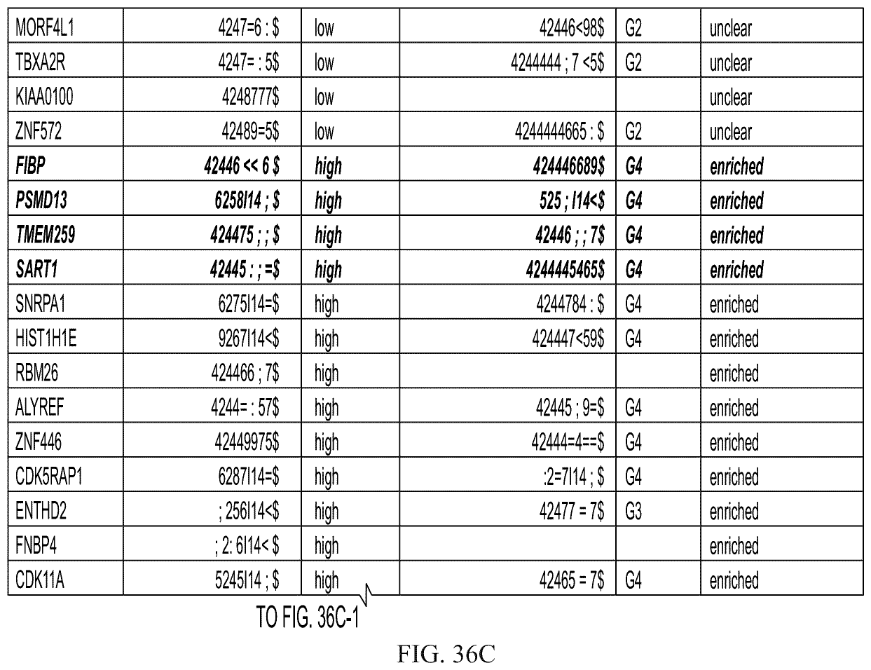

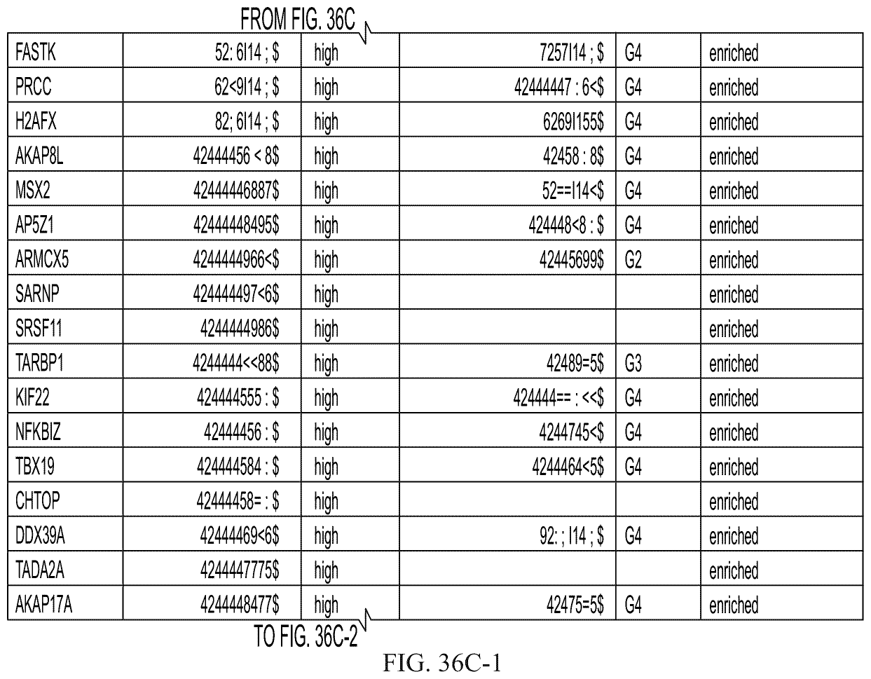

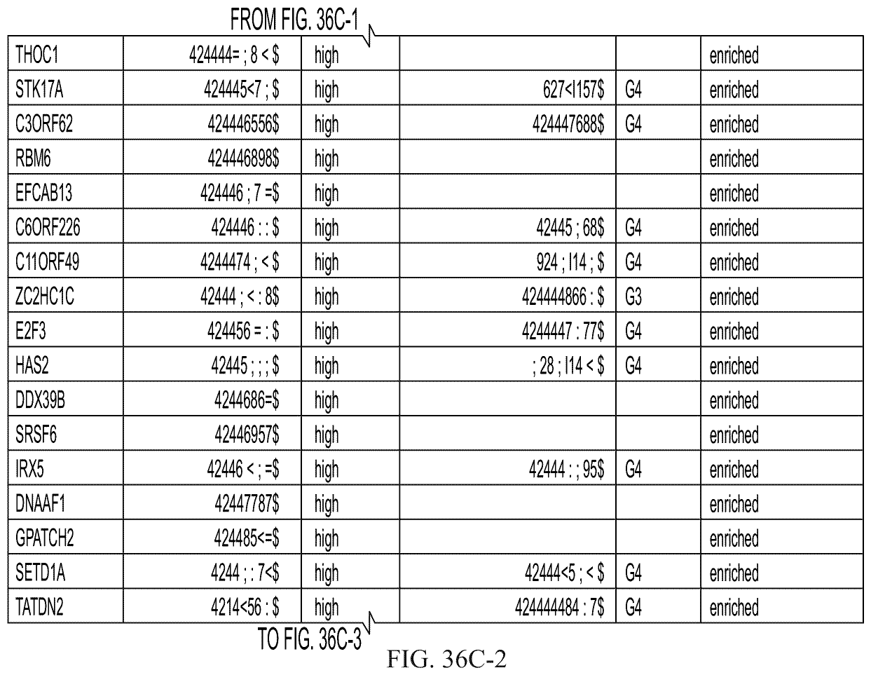

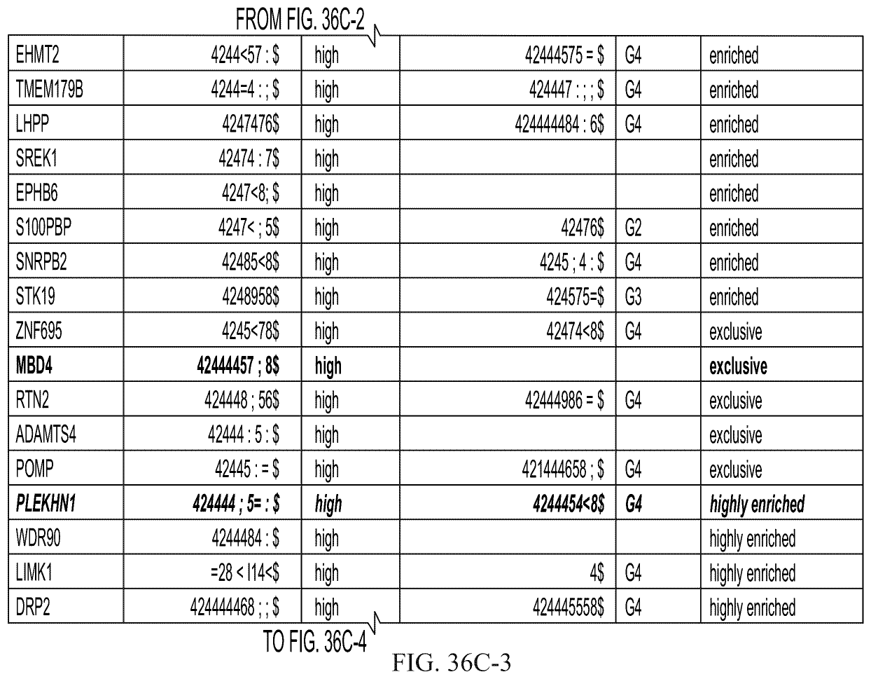

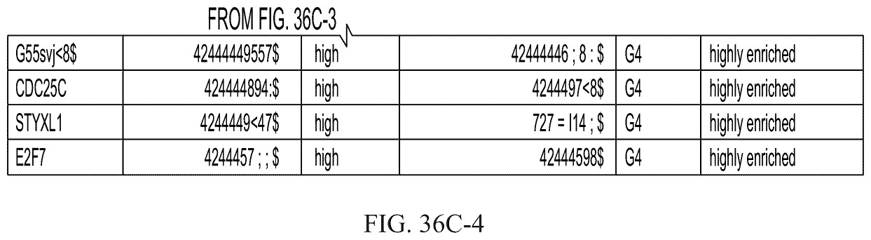

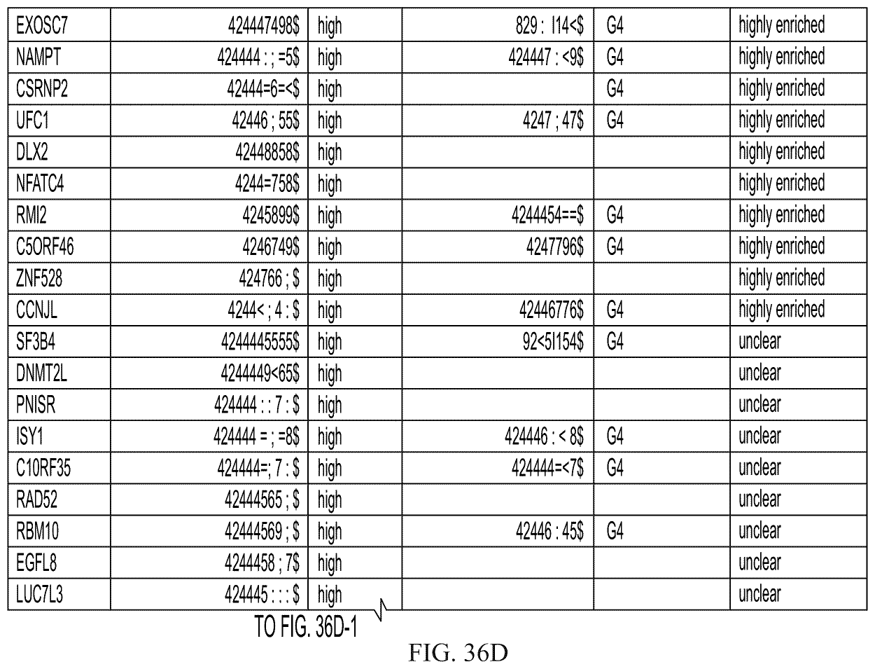

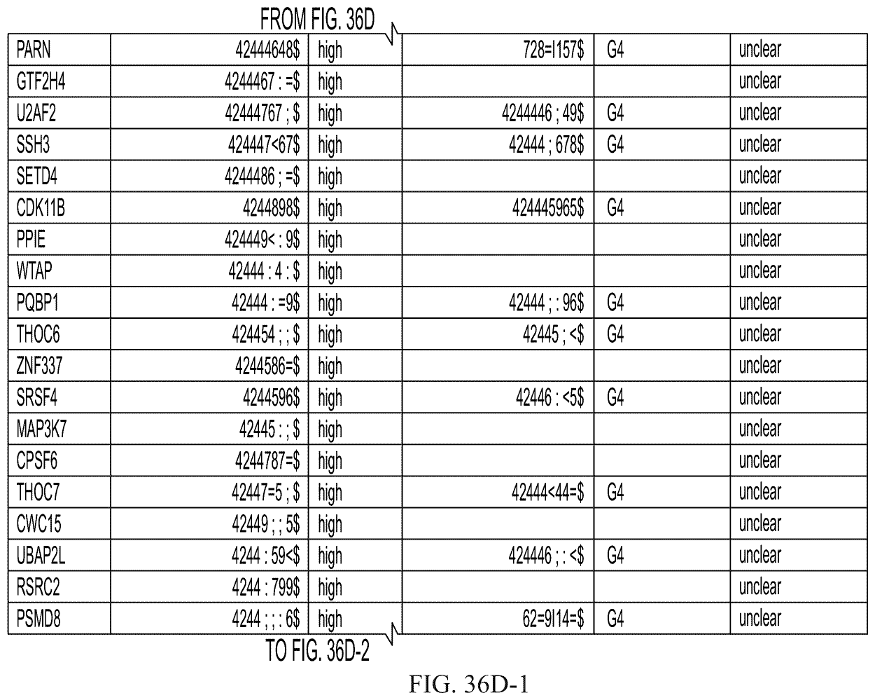

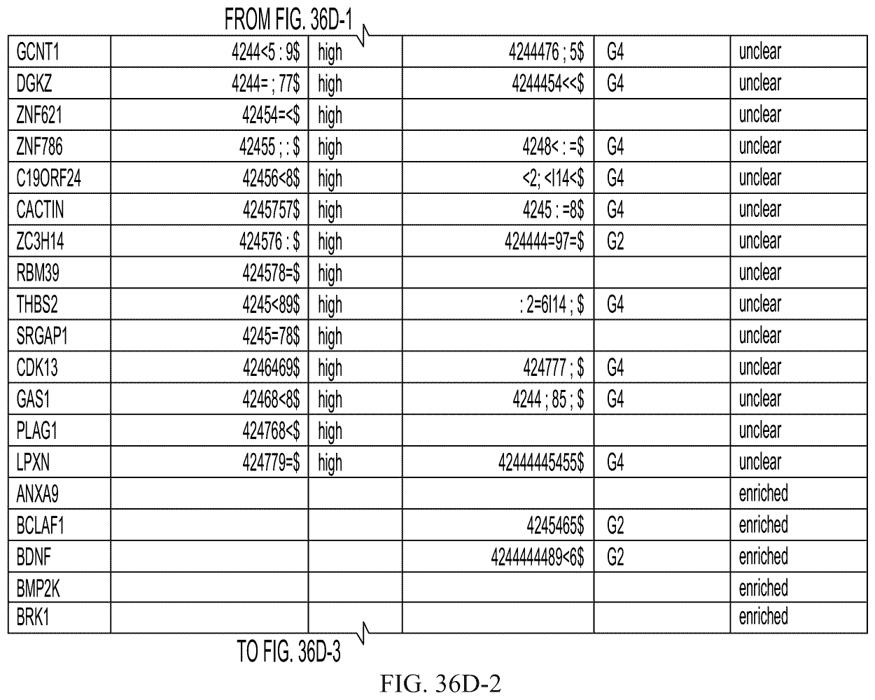

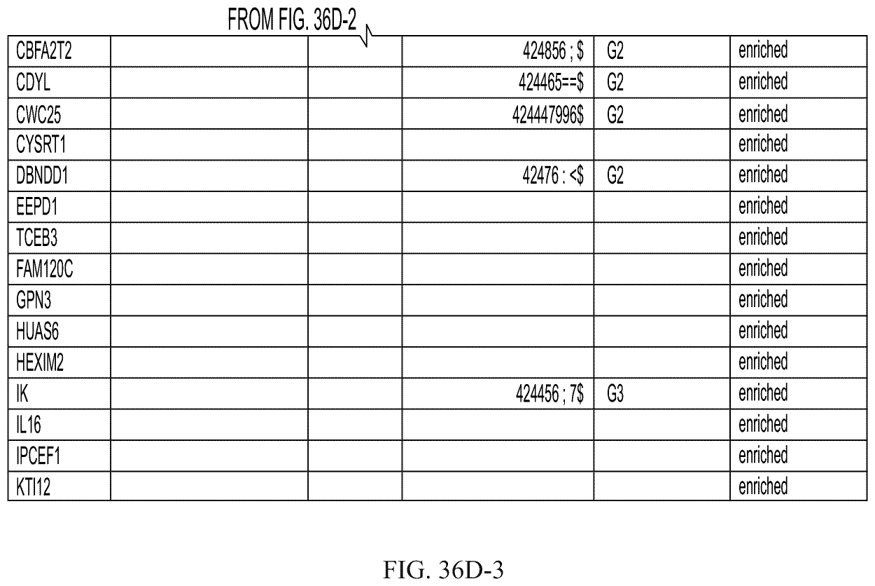

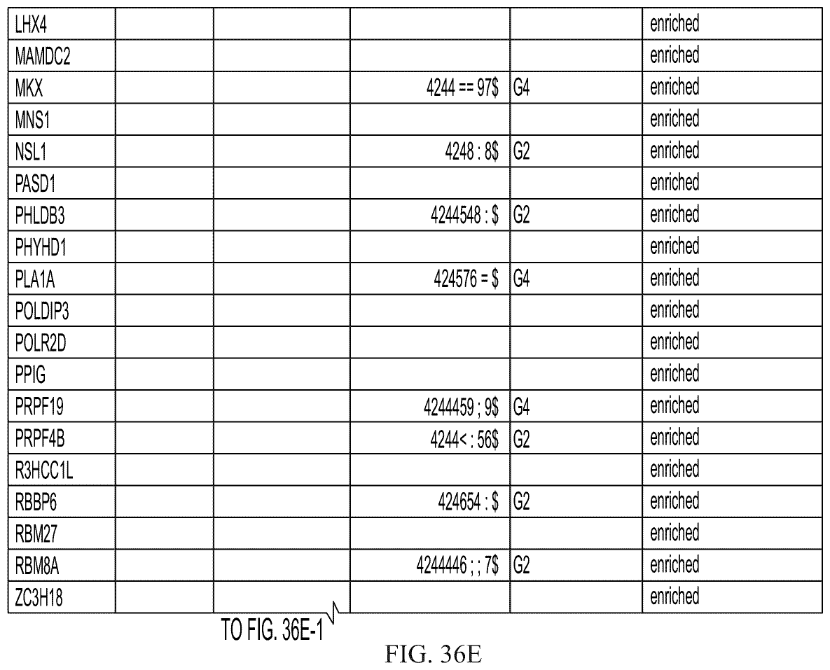

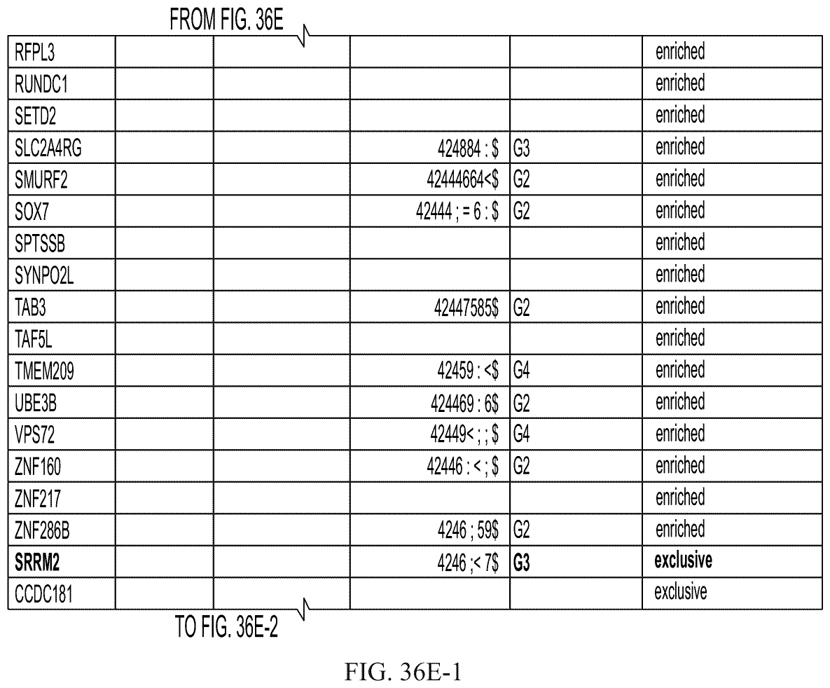

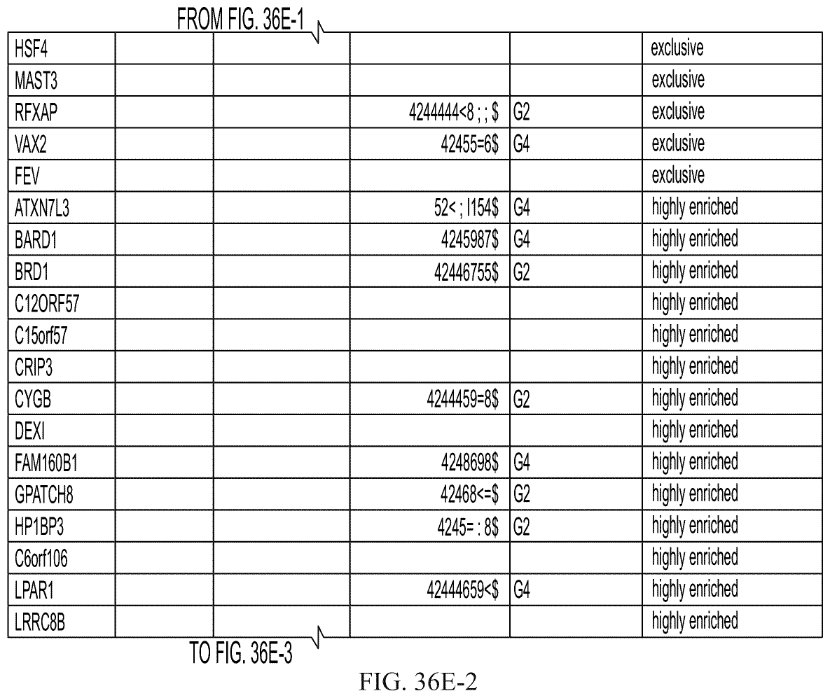

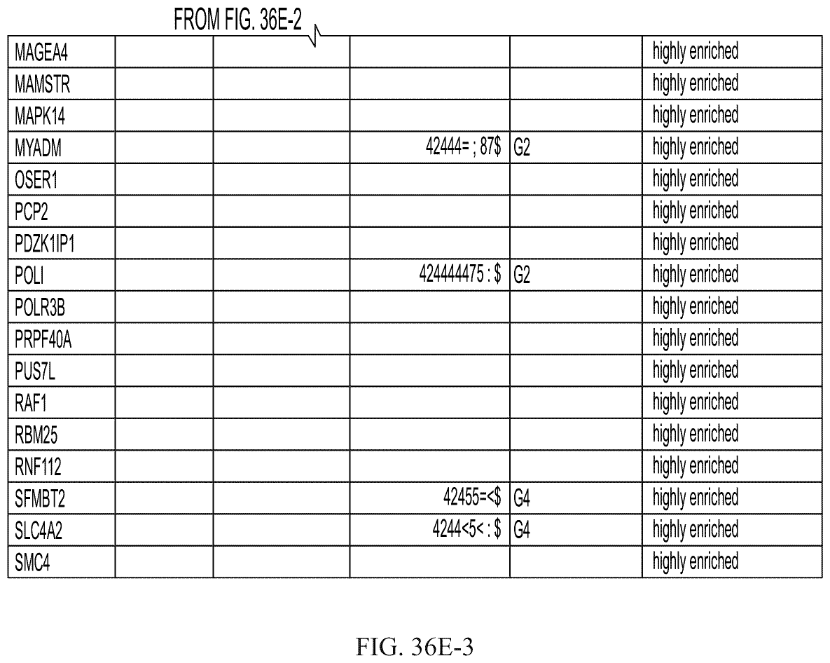

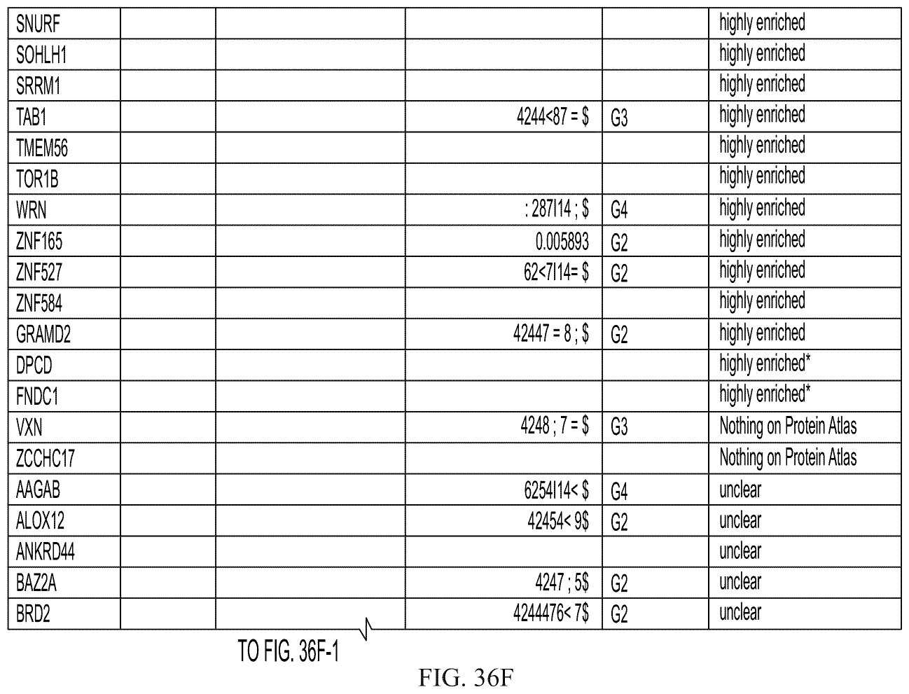

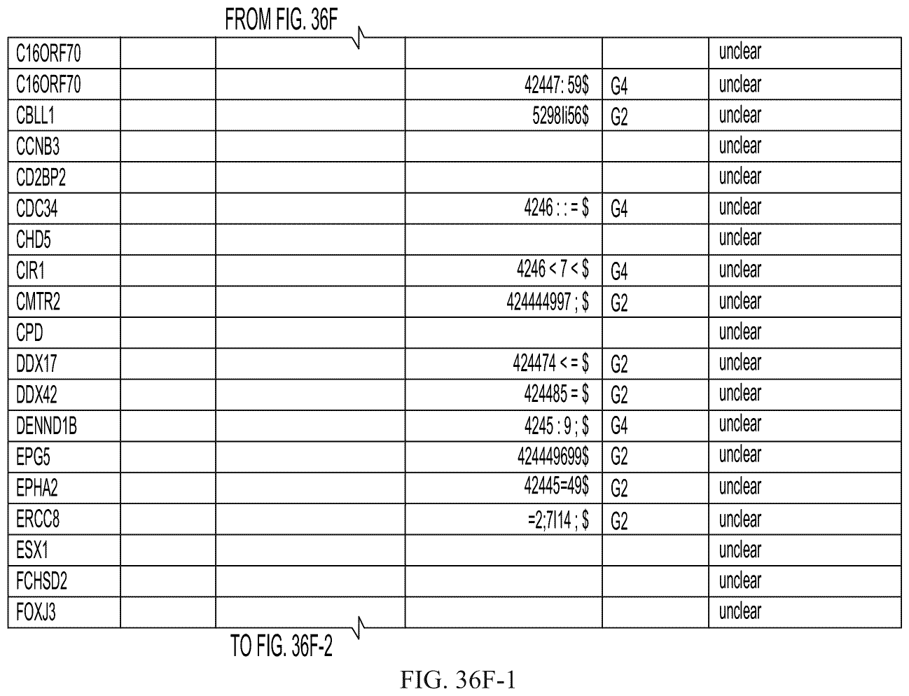

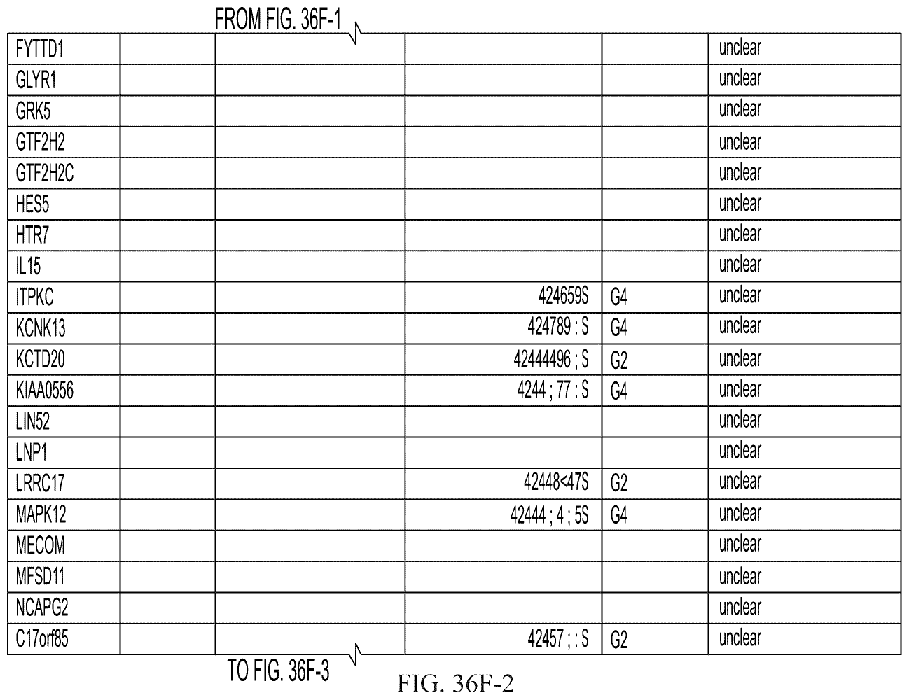

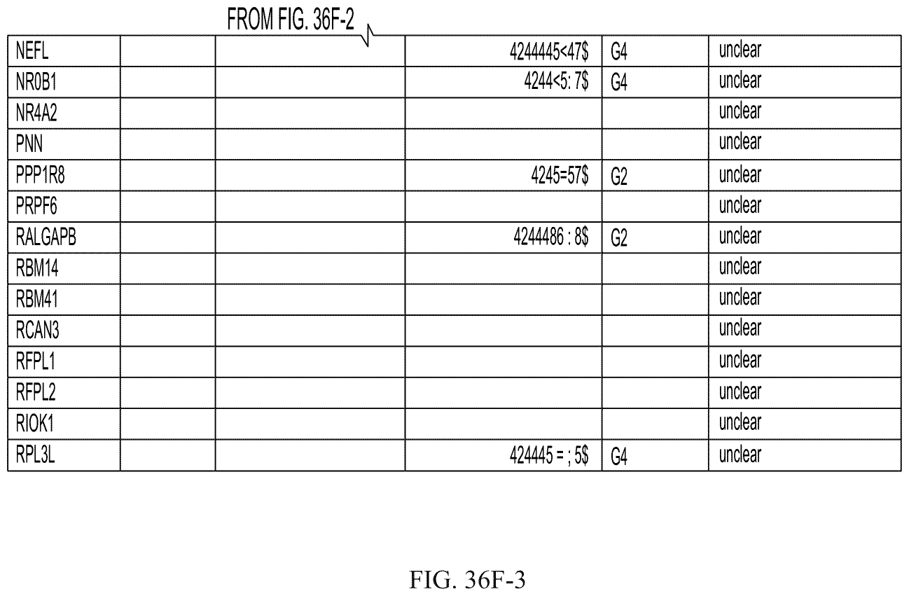

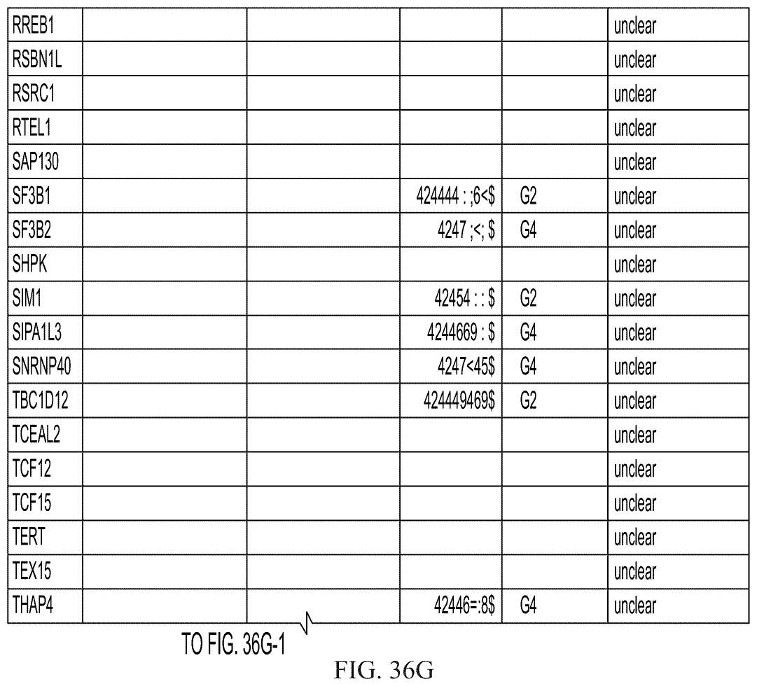

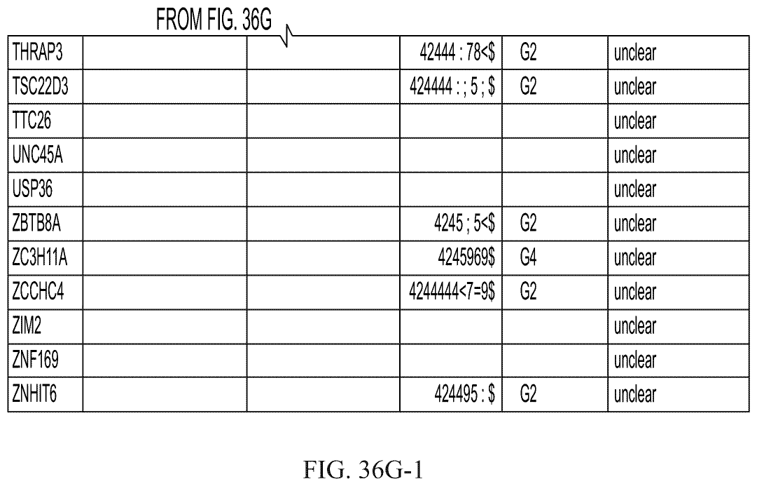

The findings presented herein provide the basis for one of the key methods for the present invention: using speckle manipulations to shift the speckle signature. An RNA-seq comparison between 786O and A498 cells, the bioinformatic definition of the speckle signature presented herein, and the generation of a resource listing all the speckle protein genes, their individual ability to predict ccRCC survival, and accompanying manual annotations of the specificity of their speckle localization based on data from the Human Protein Atlas are presented in FIGS. 35A-36G-1. Based on this analysis, knockdown of Signature II speckle protein genes HBP1 or COPS4 were found to be capable of shifting A498 cells from a Signature II-like expression phenotype to a Signature I-like expression phenotype (FIGS. 25 and 26).

### Example 4. The Speckle Signature is a Reproducible Phenomenon Among Human Cancers and is Predictive of Survival Depending on Mutation Status

Studies disclosed herein in Experimental Example 3 establish that nuclear speckles are critical regulators of gene expression patterns that predict patient survival in ccRCC. Based on these findings, and without wishing to be bound by theory, it was hypothesized that the importance of nuclear speckles for expression phenotypes and patient outcomes extends well beyond ccRCC and may be a novel therapeutic target for many cancer types.

### The Speckle Signature Exists Among Many Cancer Types.

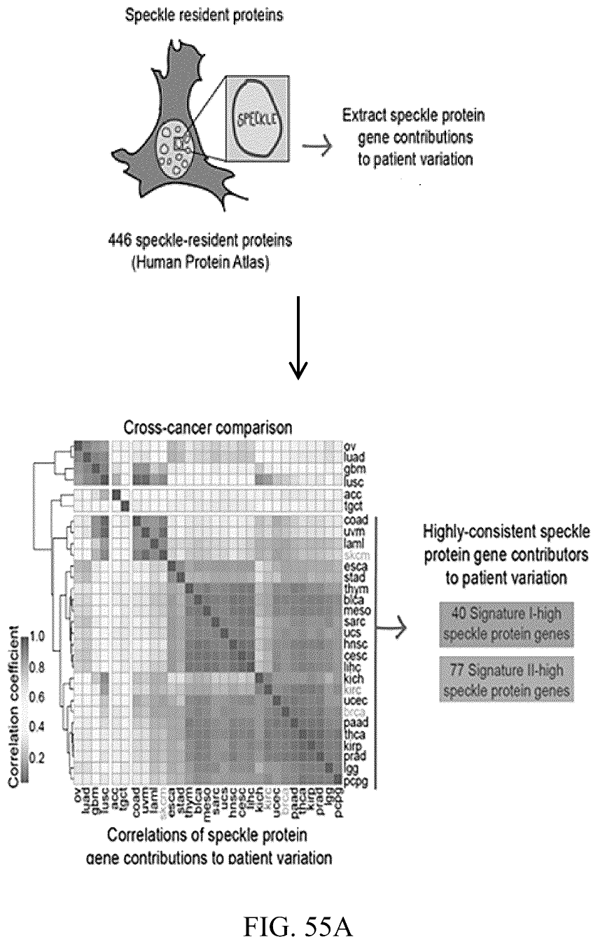

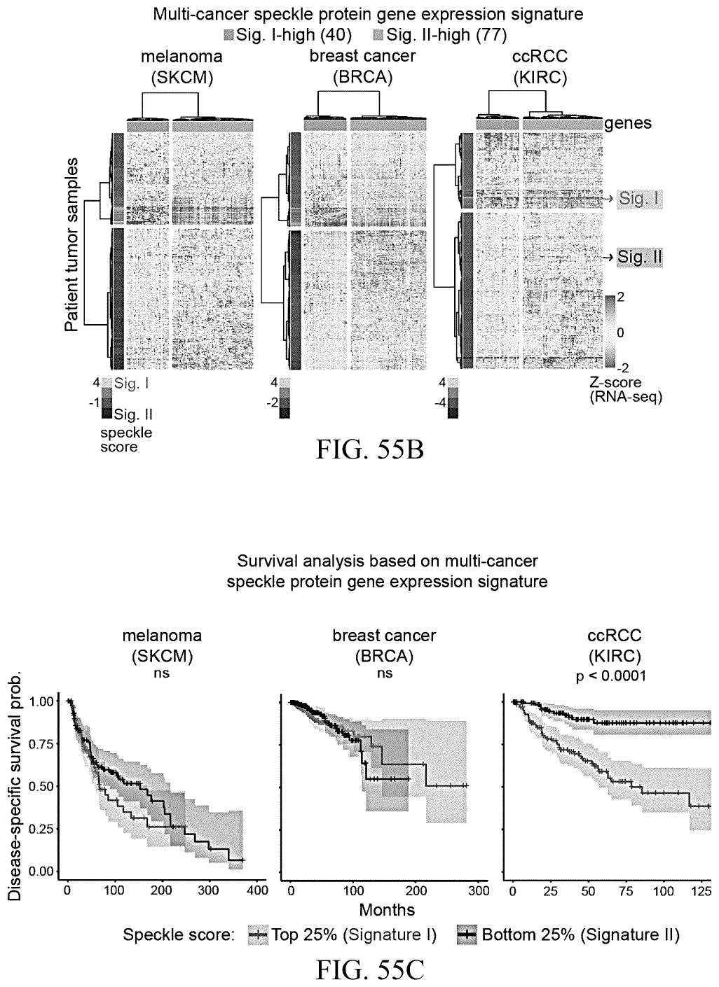

Although speckle-resident proteins are mutated in cancers and developmental disorders, methods to systematically evaluate nuclear speckle phenotypes in altered states are lacking. A characterization of nuclear speckle variation was undertaken in human cancer, utilizing RNA expression of genes encoding speckle-resident proteins as a proxy for speckle phenotypes. 446 speckle-resident proteins were extracted based on speckle-localization annotations from the Human Protein Atlas (FIG. 55A) and estimated speckle phenotypes from their RNA expression in The Cancer Genome Atlas (TCGA) using Principal Component Analysis (PCA). Comparing speckle protein gene expression contributions to patient variation between cancer types (derived from PCA analysis), remarkable correlations were observed between cancer types (FIG. 55A, strong correlations are orange and red), indicating that speckle protein gene expression varies reproducibly in cancer.

Based on this consistent speckle protein gene expression variation across many cancer types, a multi-cancer 117 gene “speckle signature” was generated containing speckle protein genes that consistently contributed to patient variation (FIG. 55A). This included 40 “Signature I-high” speckle protein genes and 77 “Signature 11-high” speckle protein genes that were consistently reciprocally expressed, and that separated tumor samples into two groups (FIG. 55B). Each patient was assigned a speckle signature score based on the collective expression of these 117 speckle protein genes (FIG. 55B, speckle score on the left colored column of each heatmap) and used this quantitative measure for Kaplan-Meier survival analysis. Overall and disease-specific survival was assessed, separating patients by the top versus bottom quartiles of speckle scores. Of 24 cancers with highly consistent speckle protein gene contributions to patient variation (right grey bar in FIG. 55A), 21 showed no correlation between speckle signature and patient outcomes for any survival measurement, as shown by examples of melanoma (SKCM) and breast cancer (BRCA) (FIG. 55C, left panels), and two, ovarian (OV) and head and neck cancer (HNSC), showed modest survival correlations.

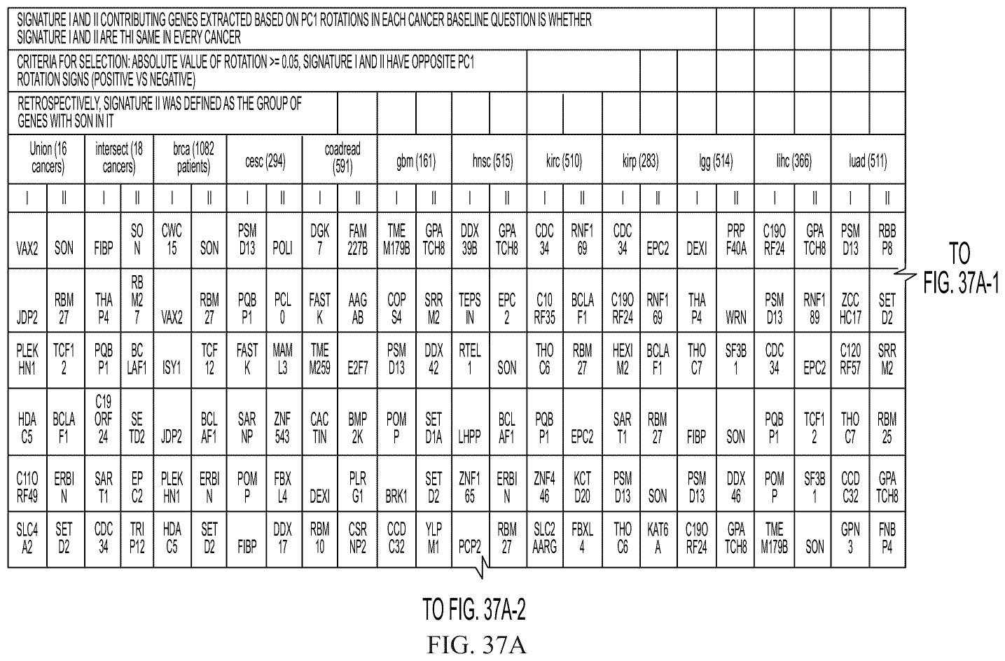

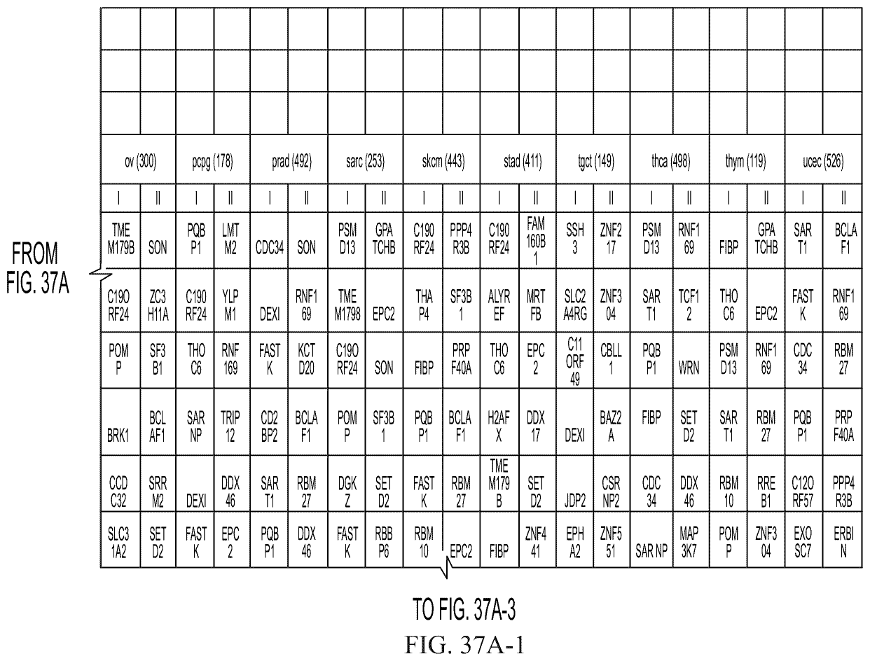

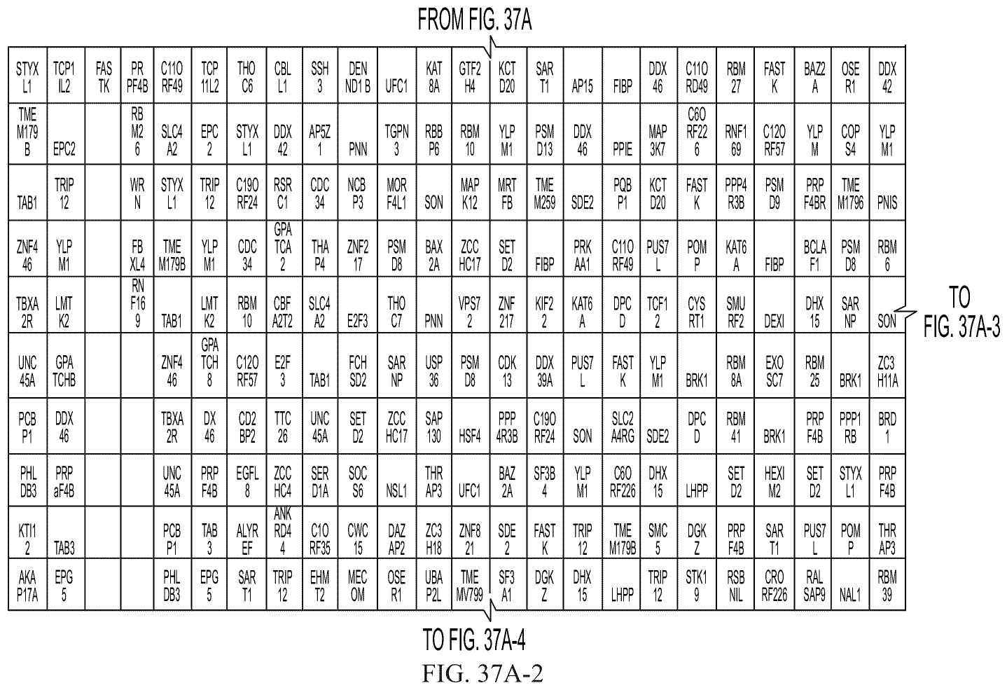

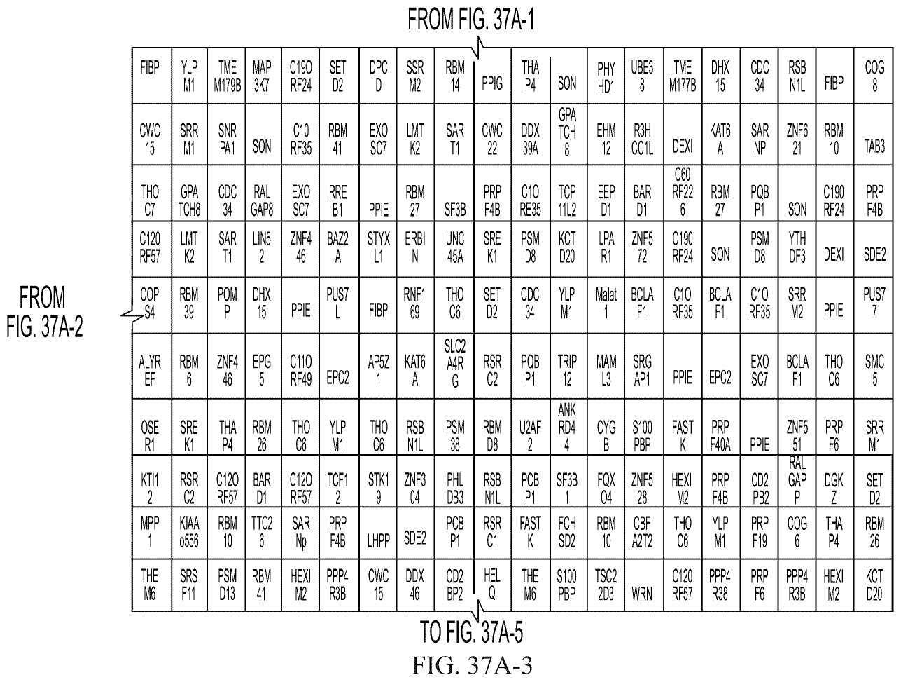

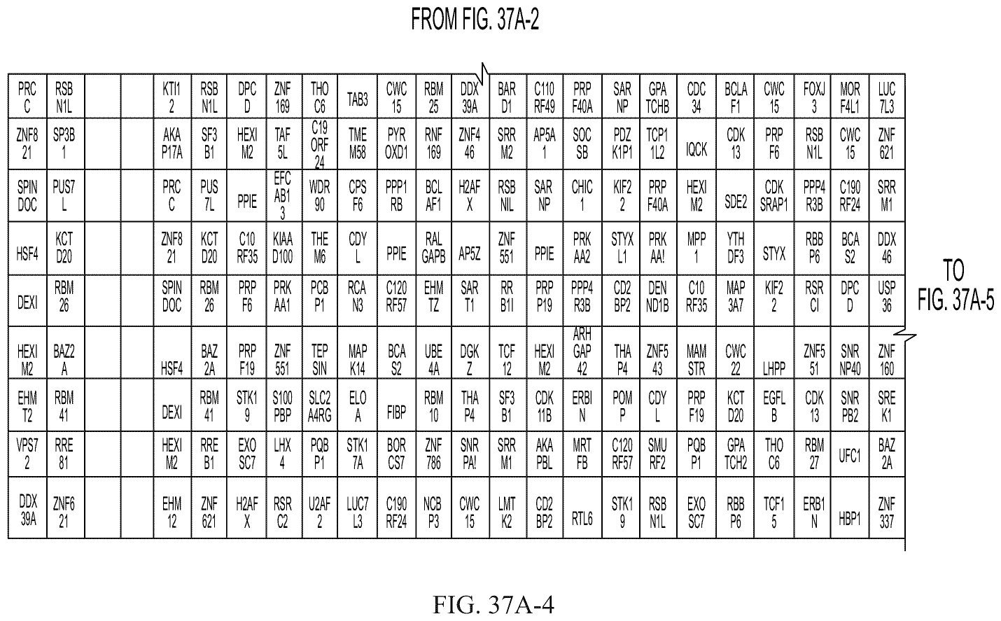

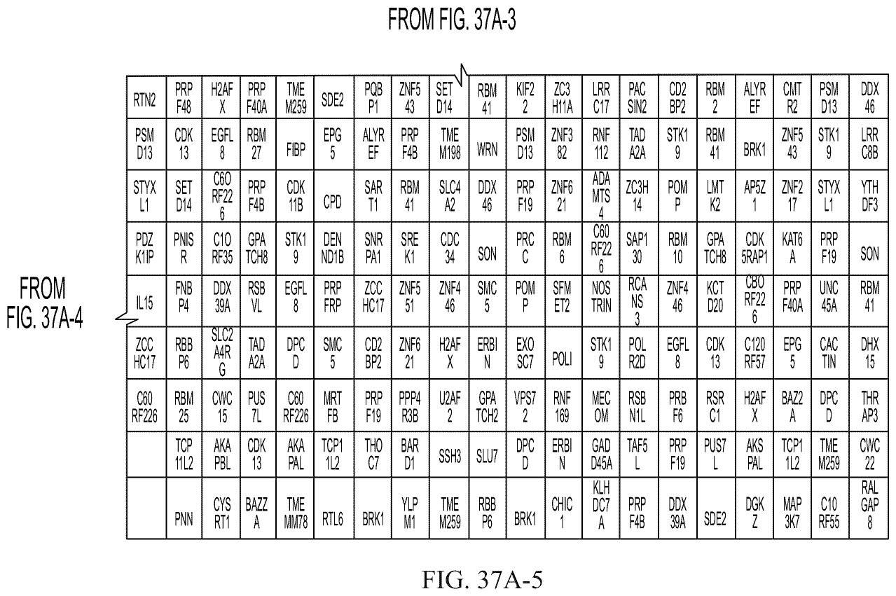

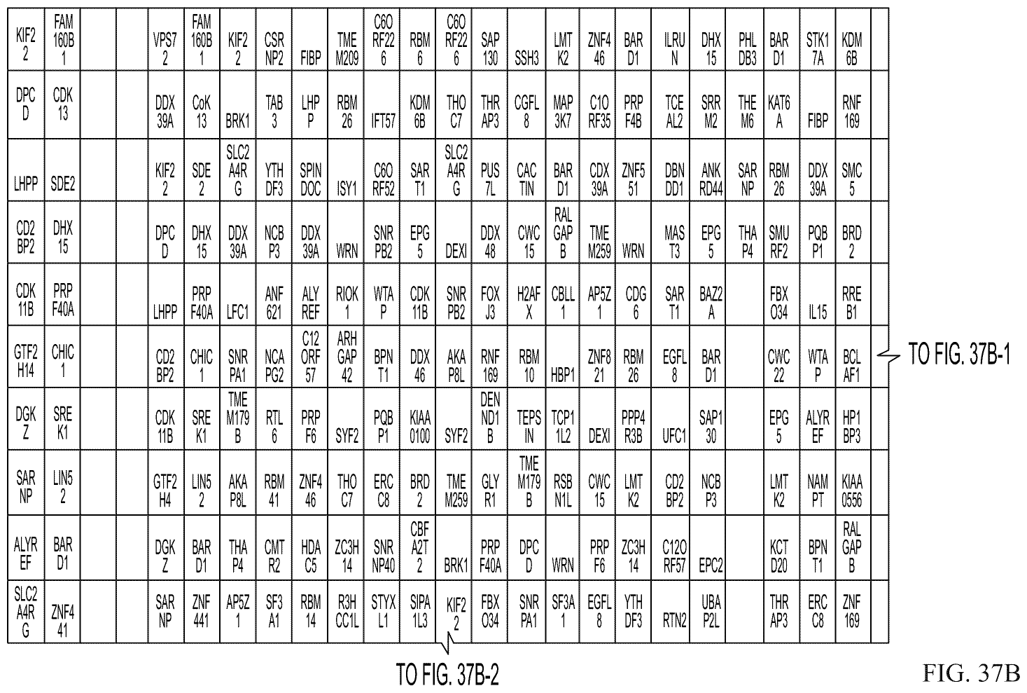

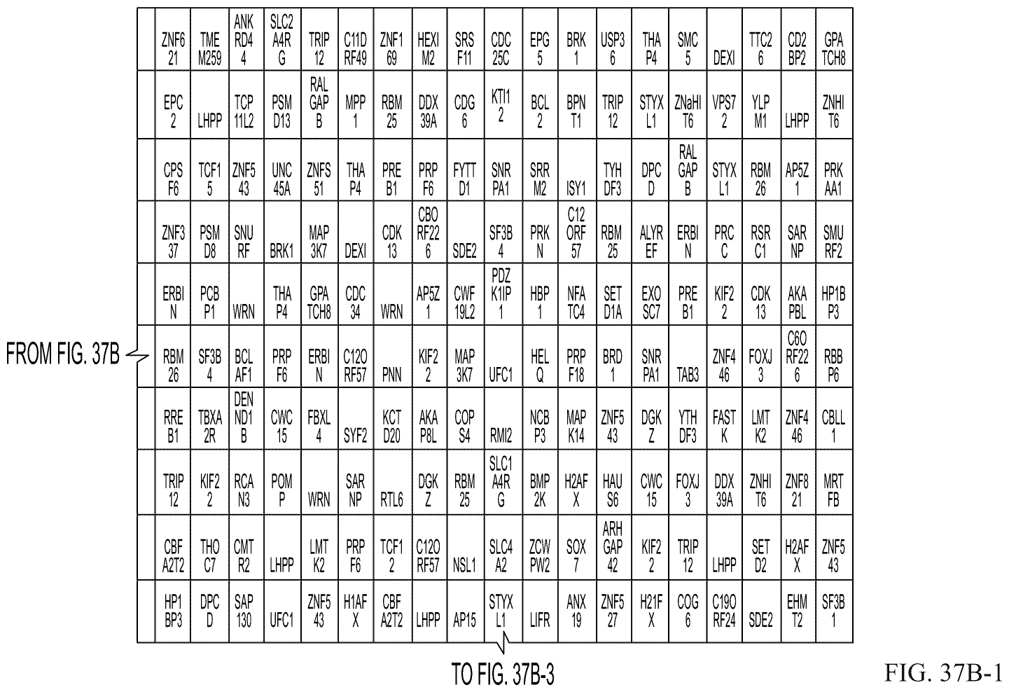

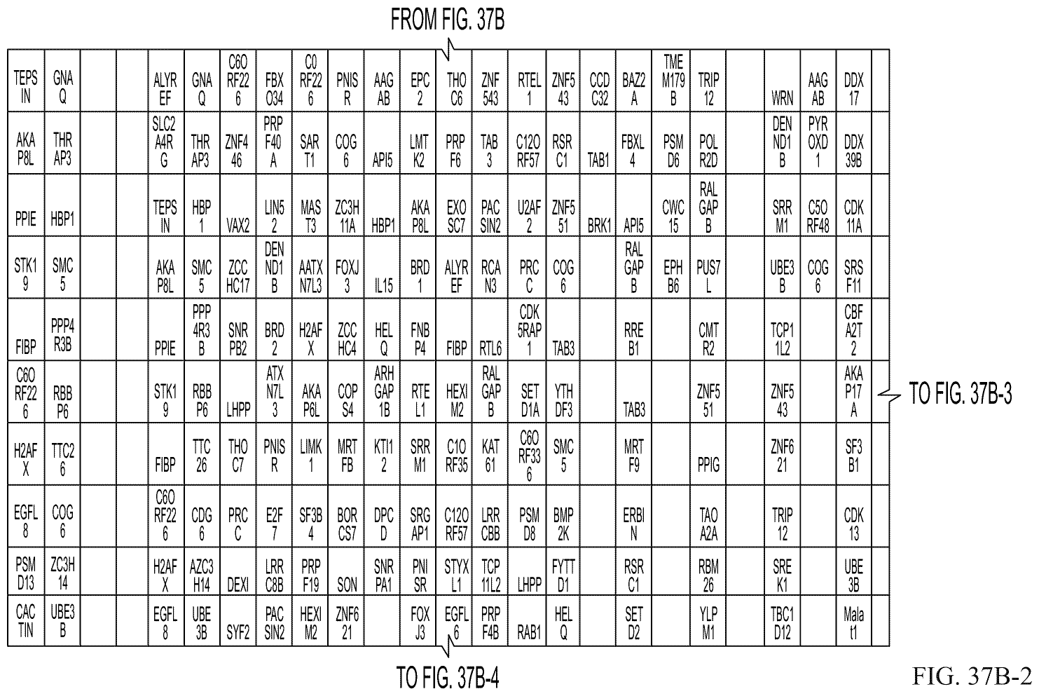

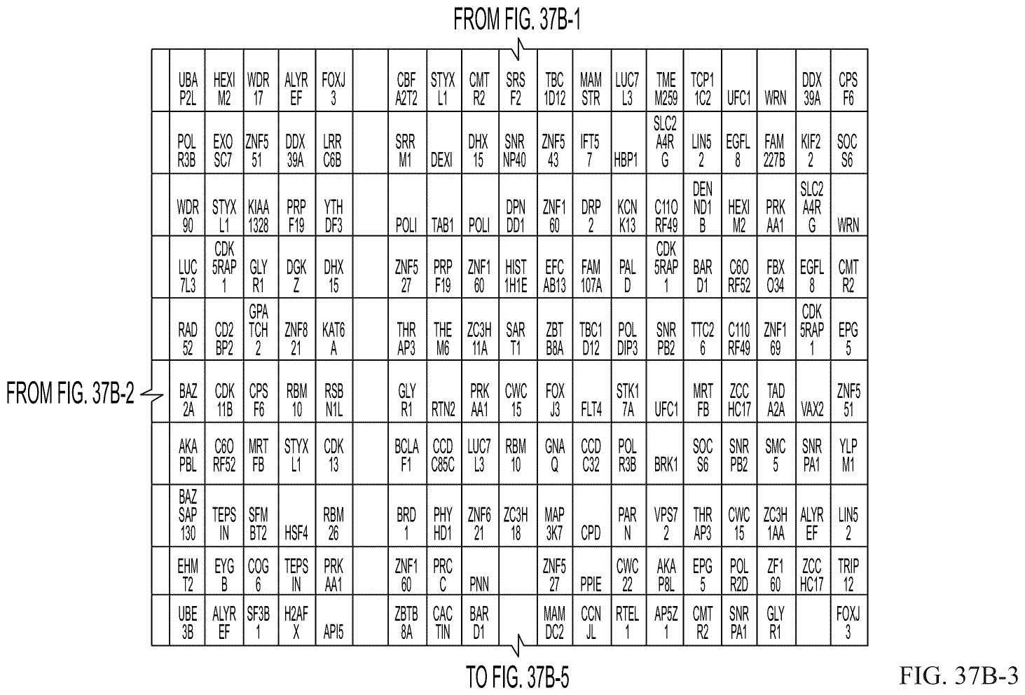

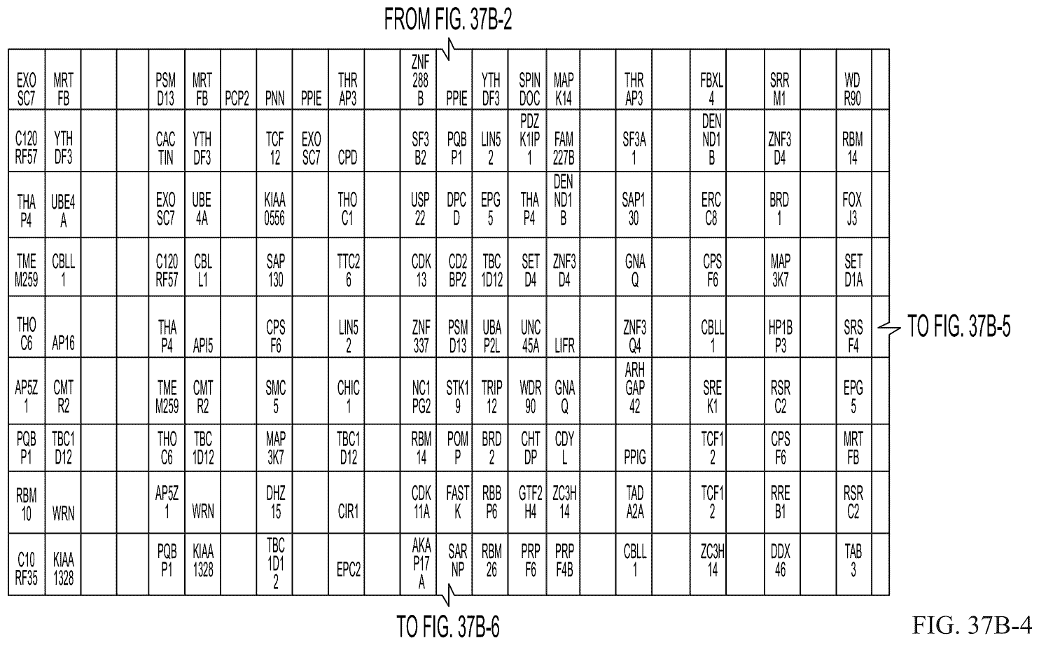

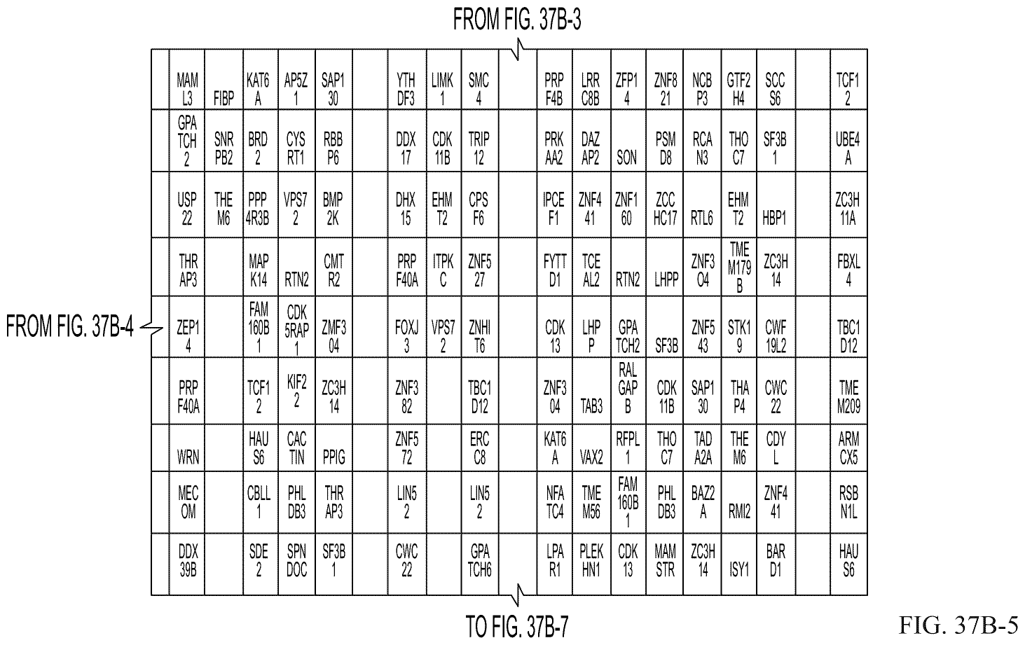

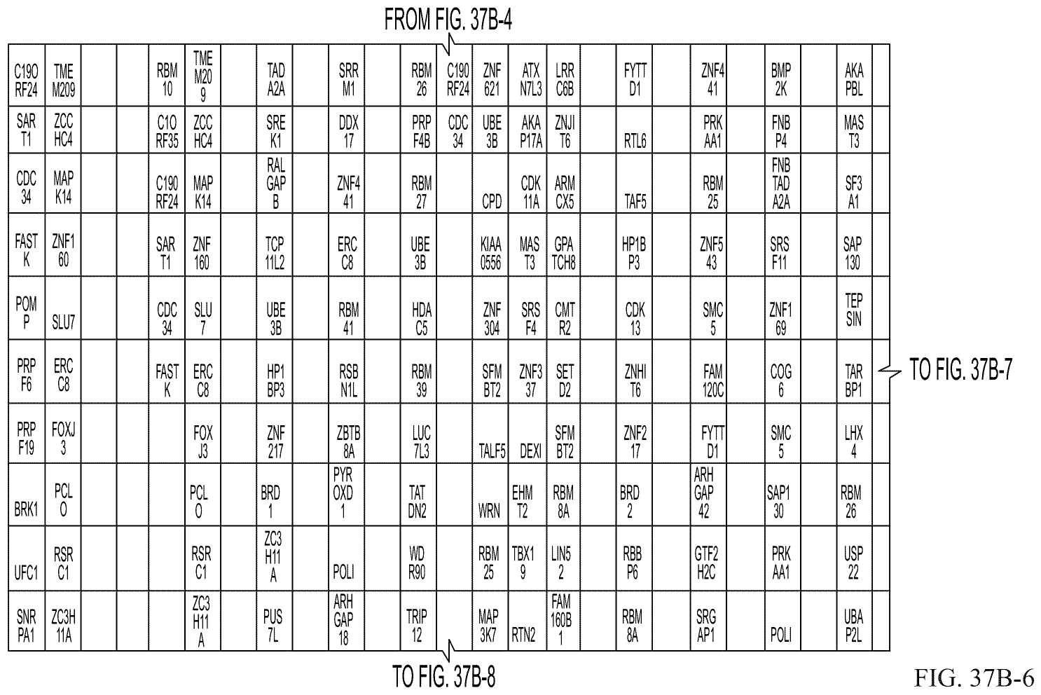

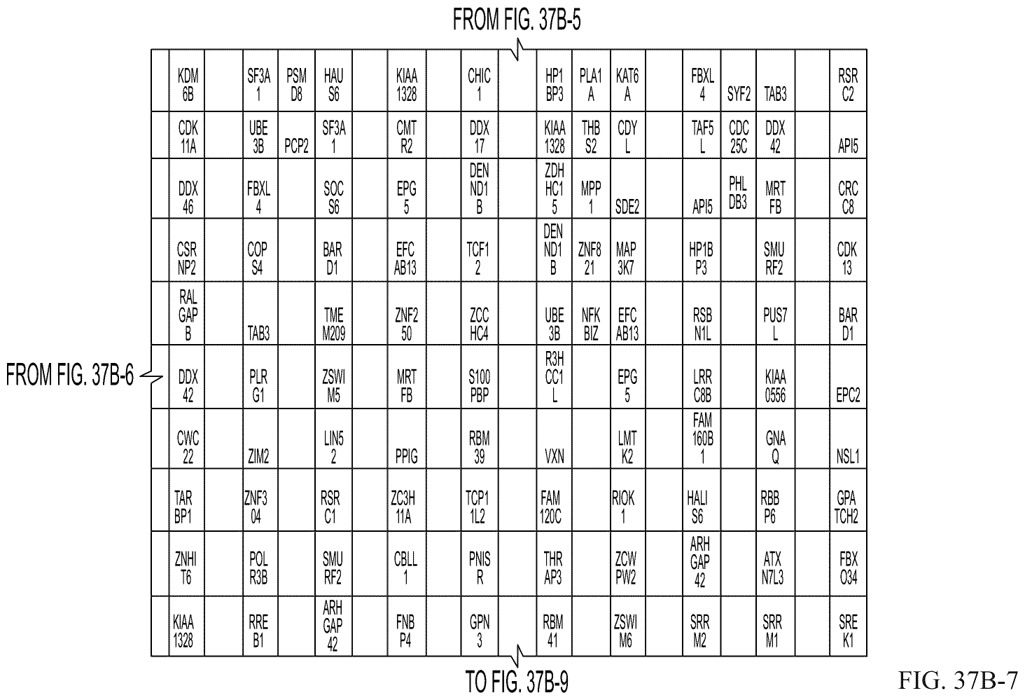

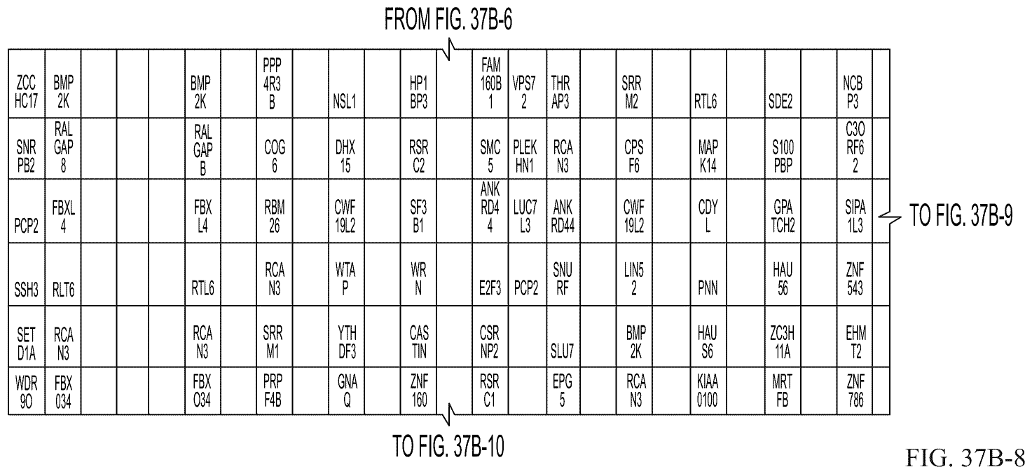

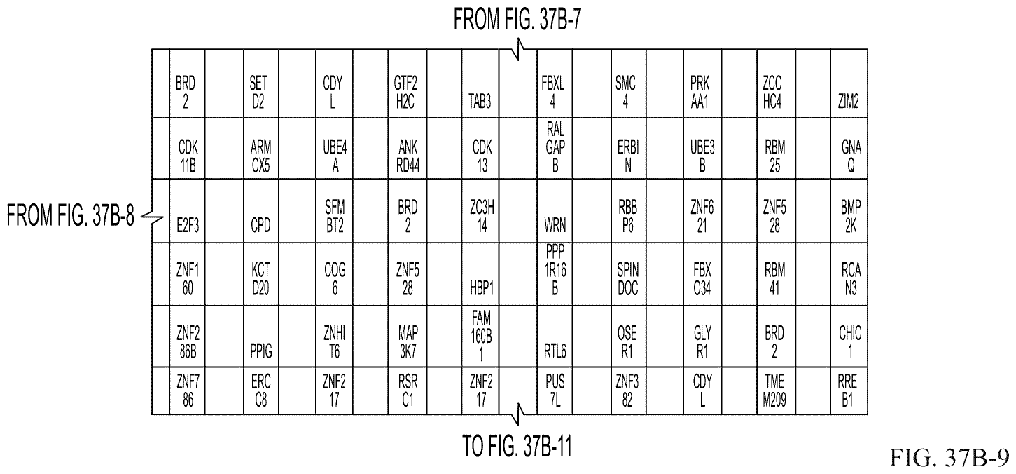

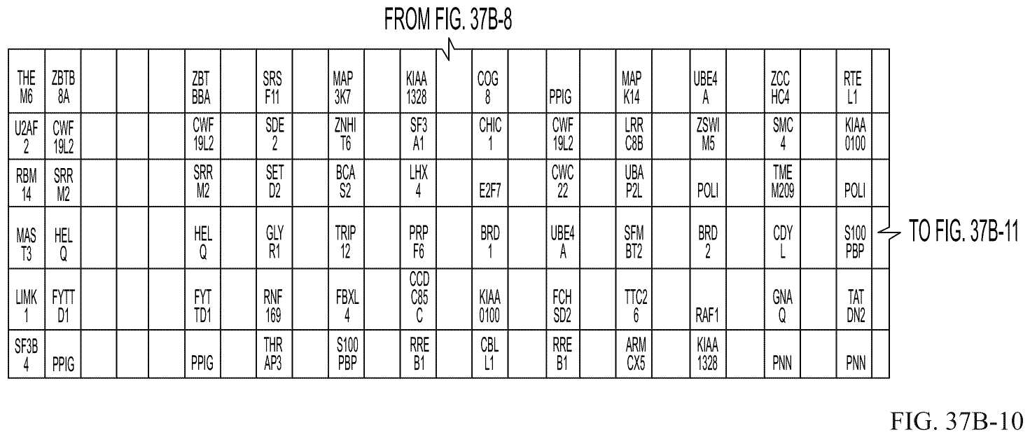

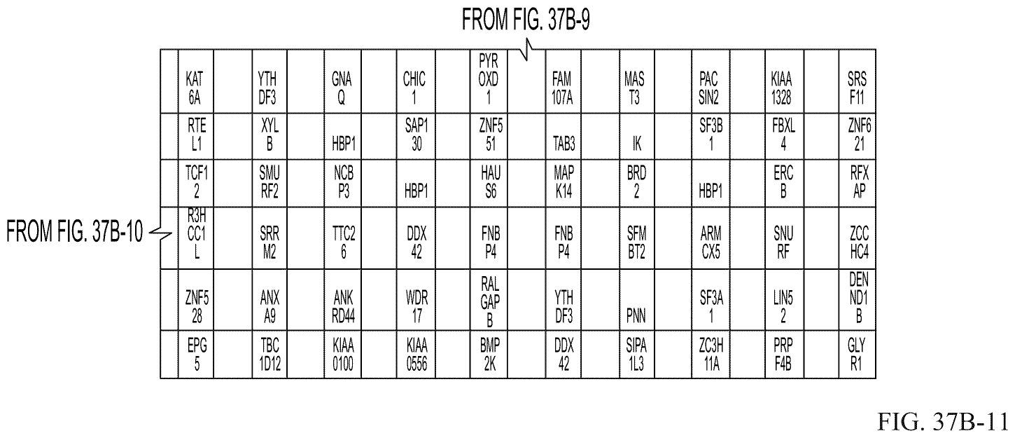

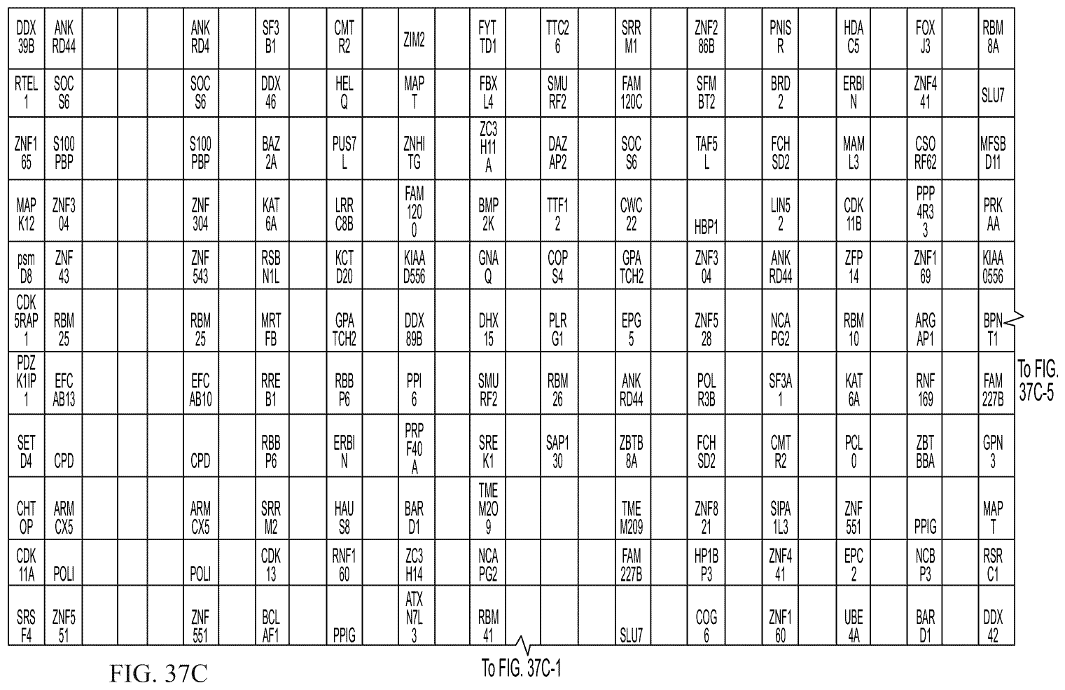

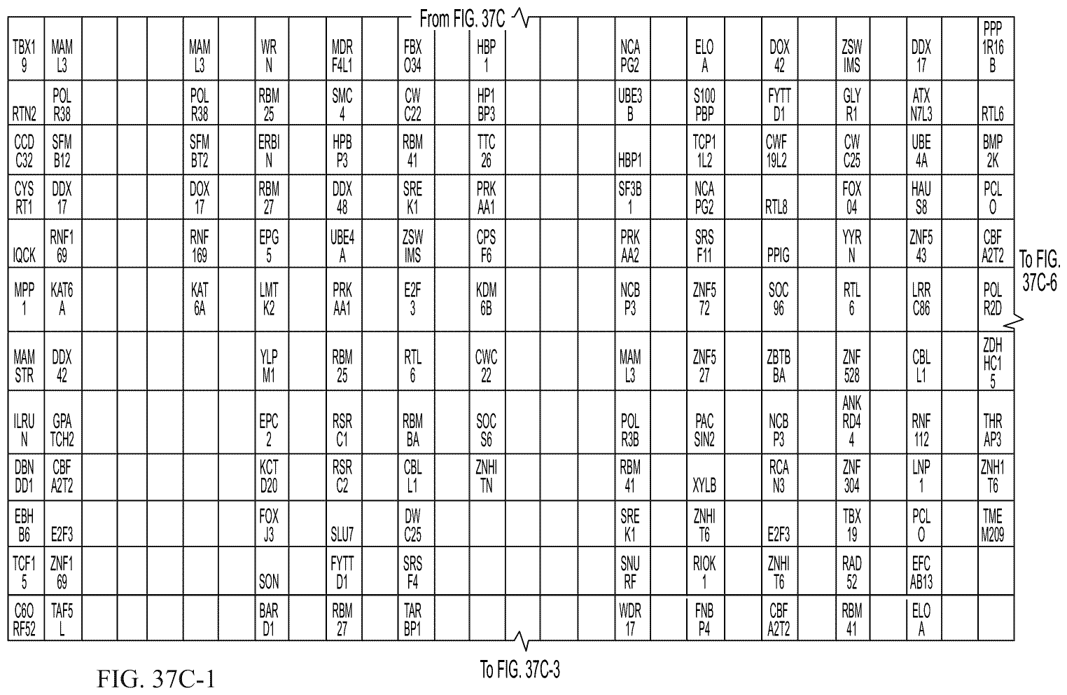

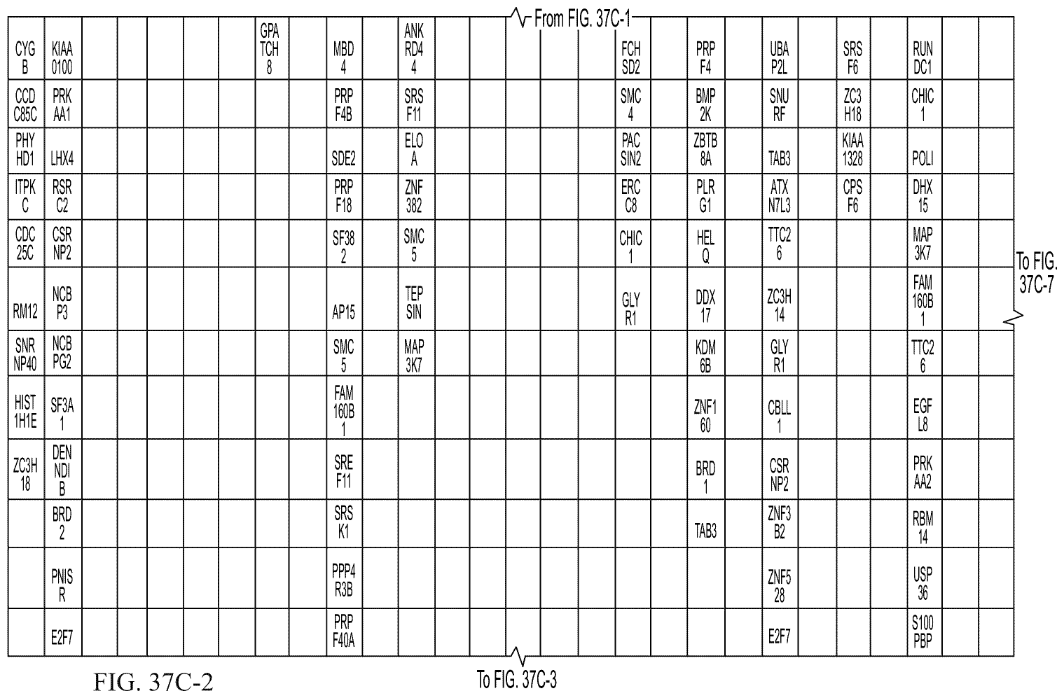

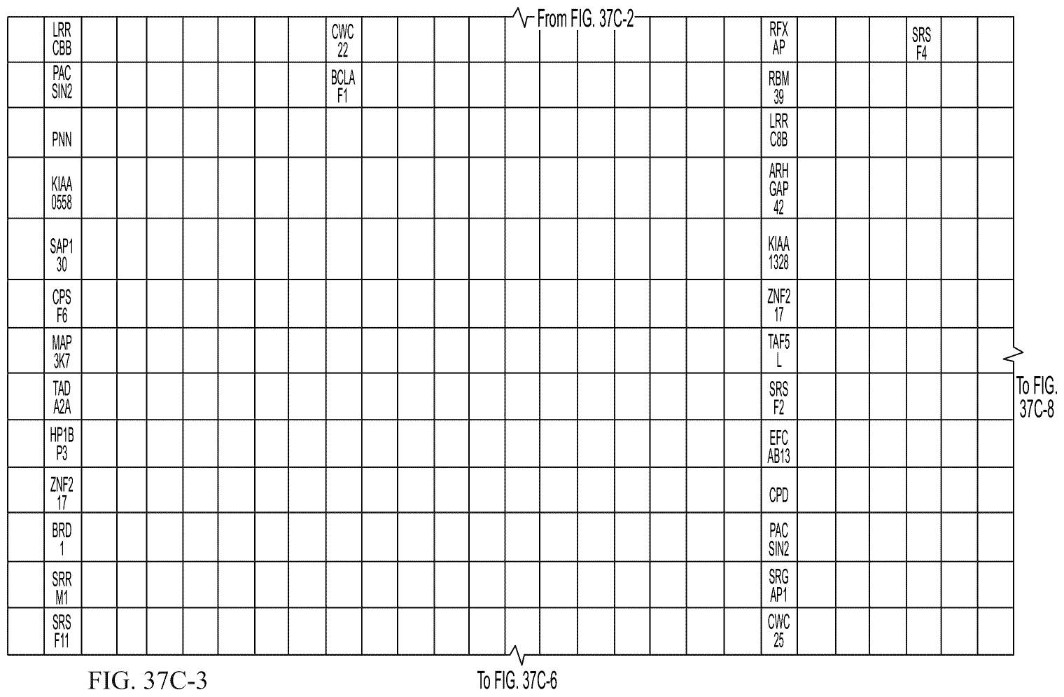

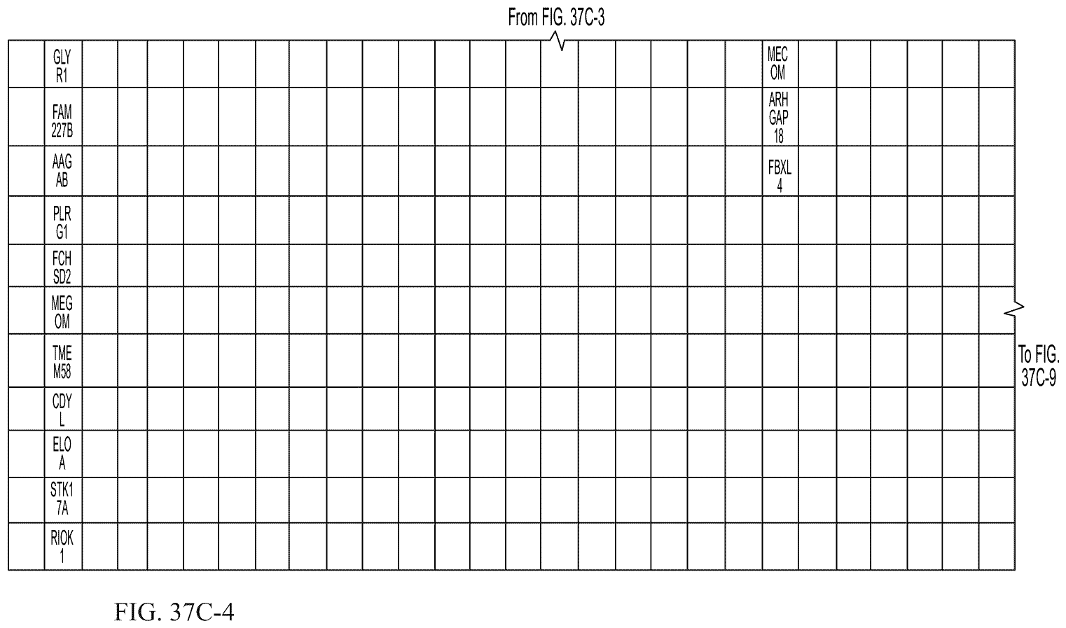

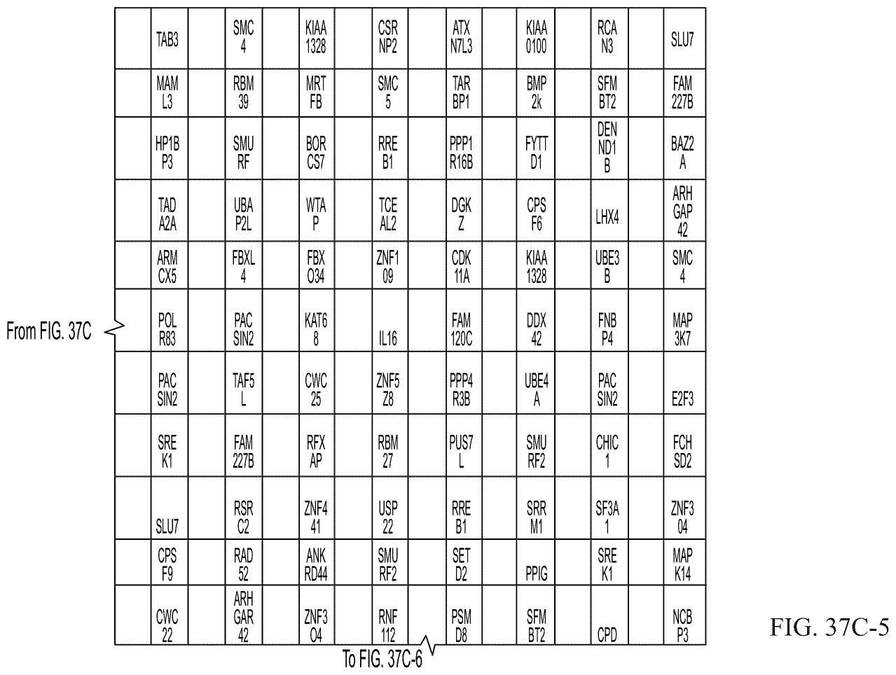

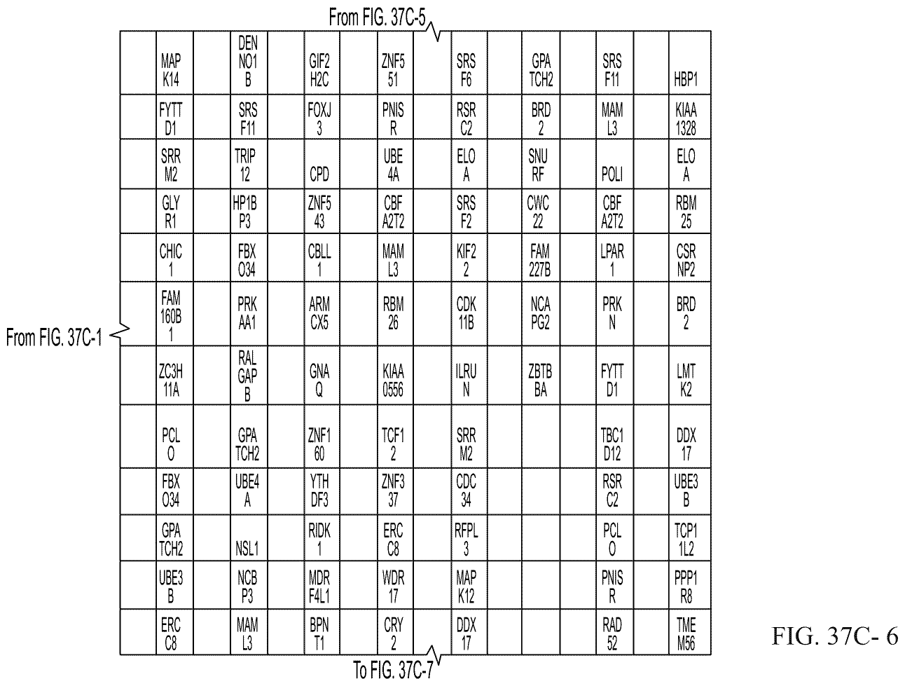

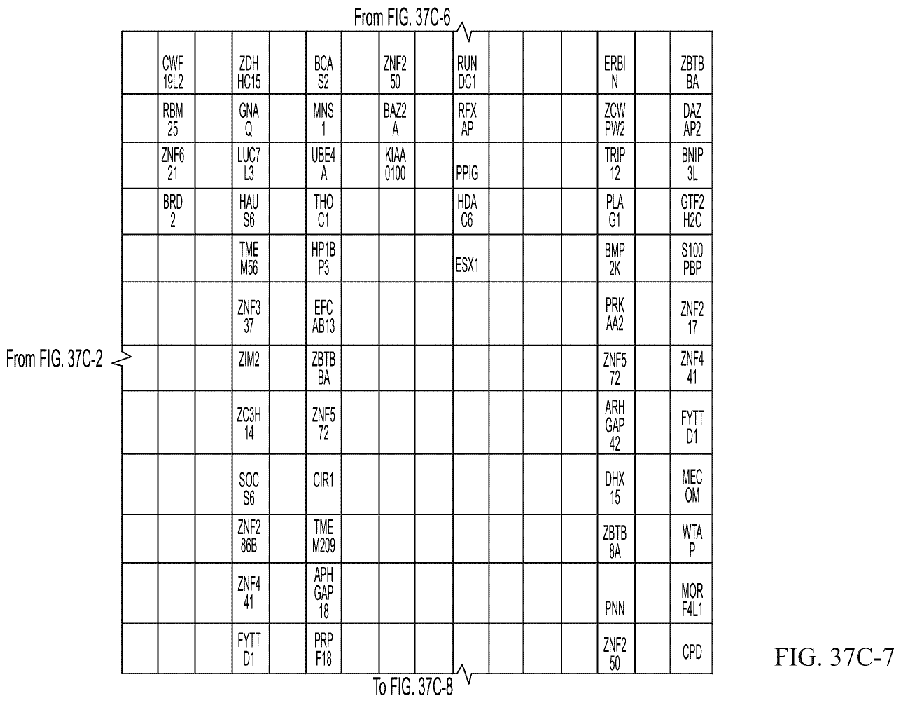

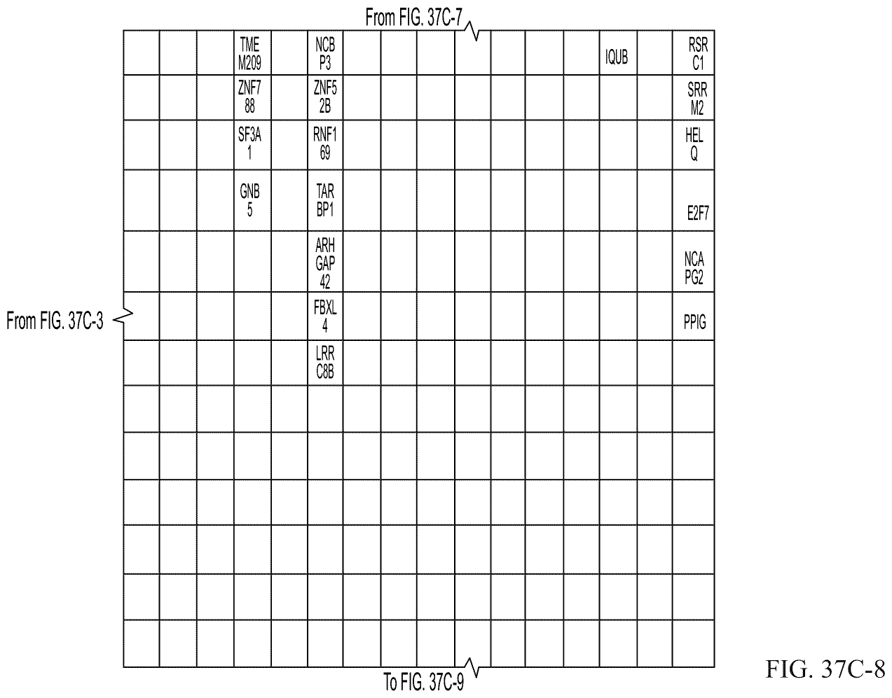

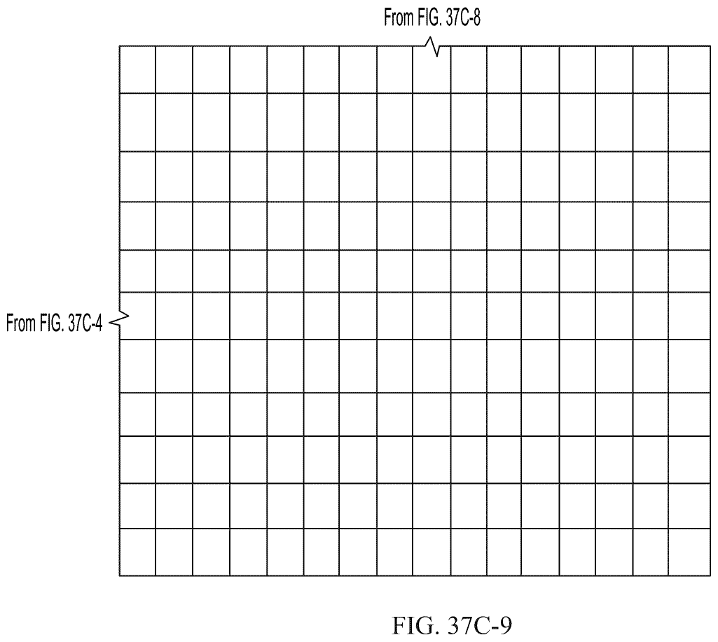

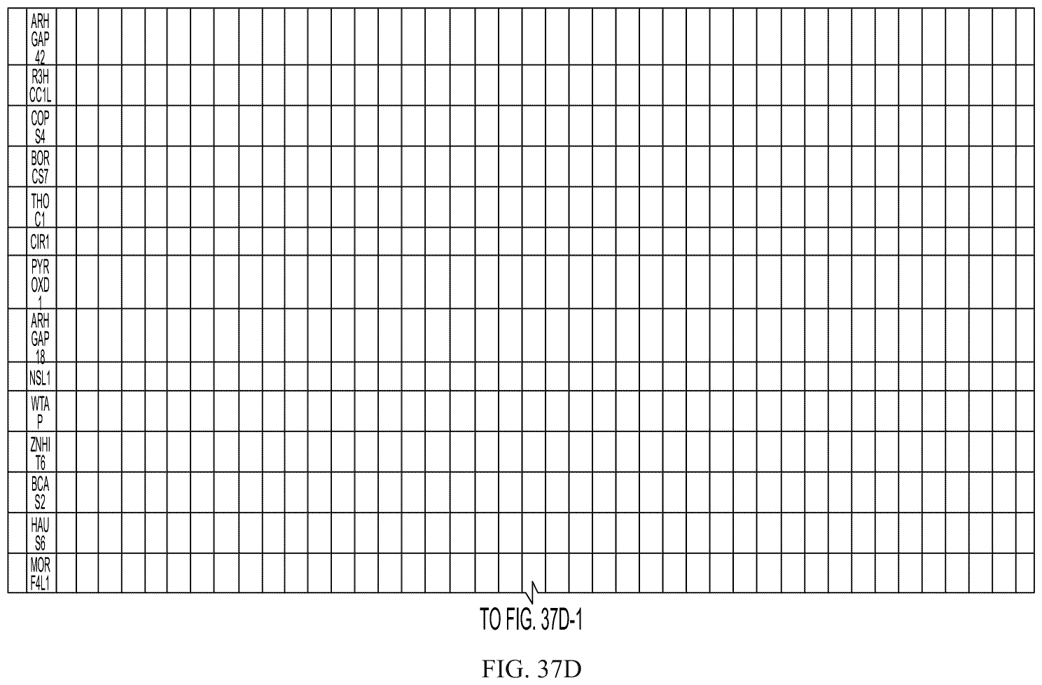

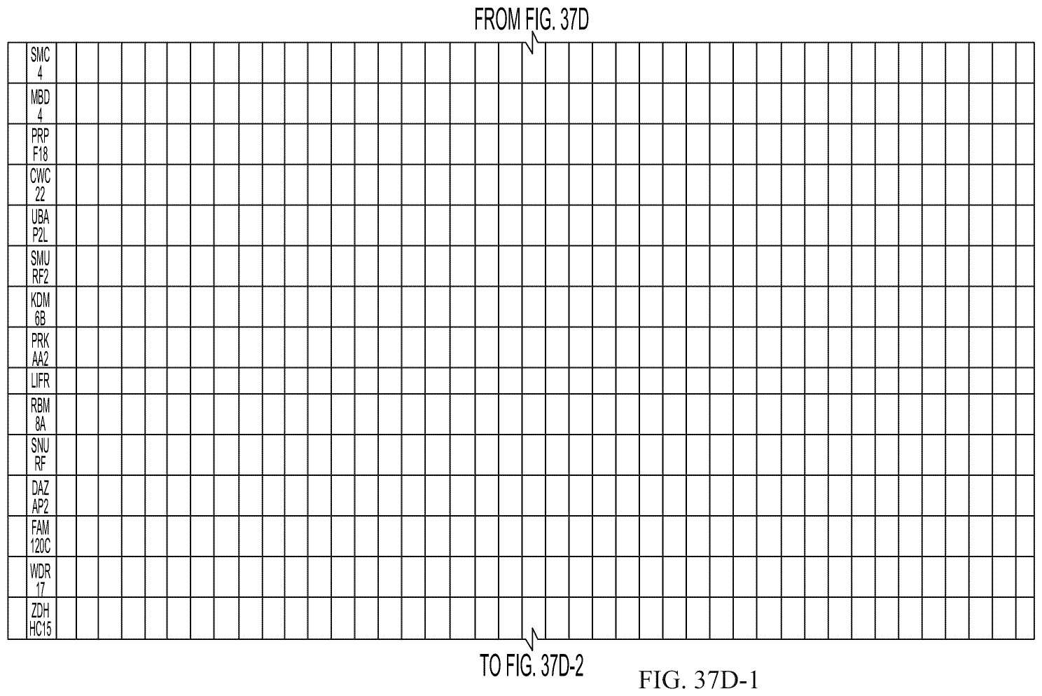

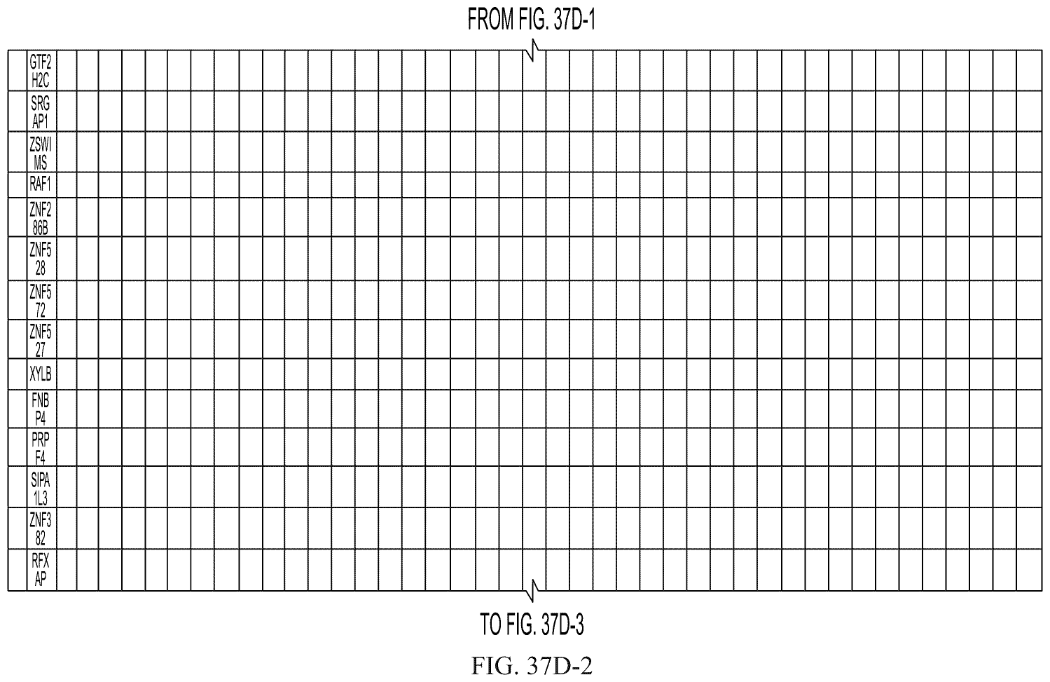

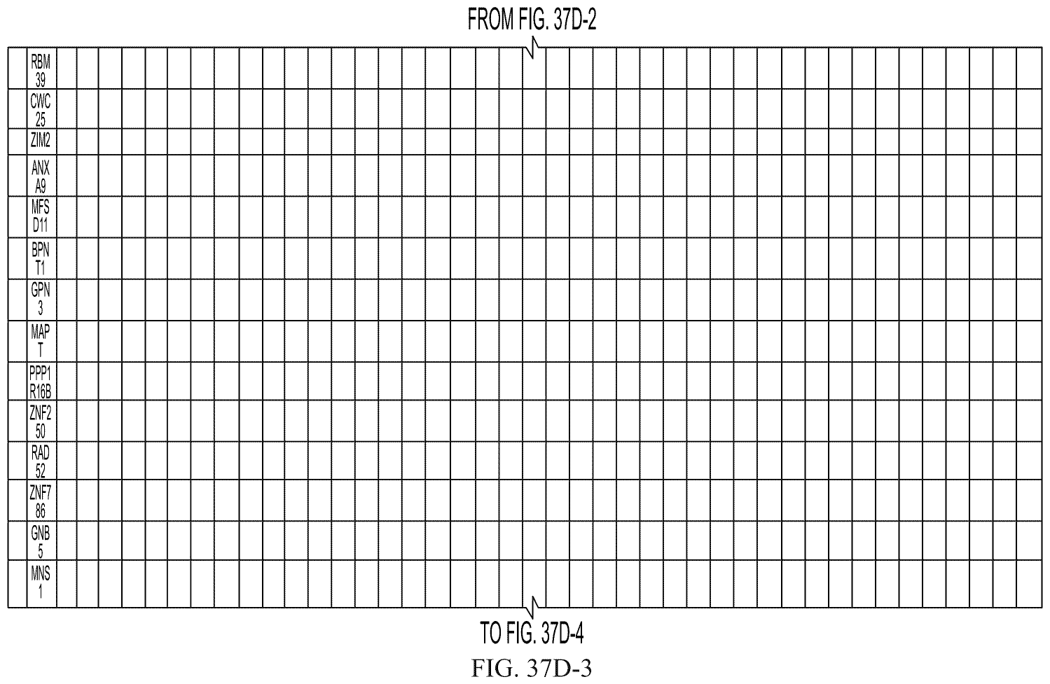

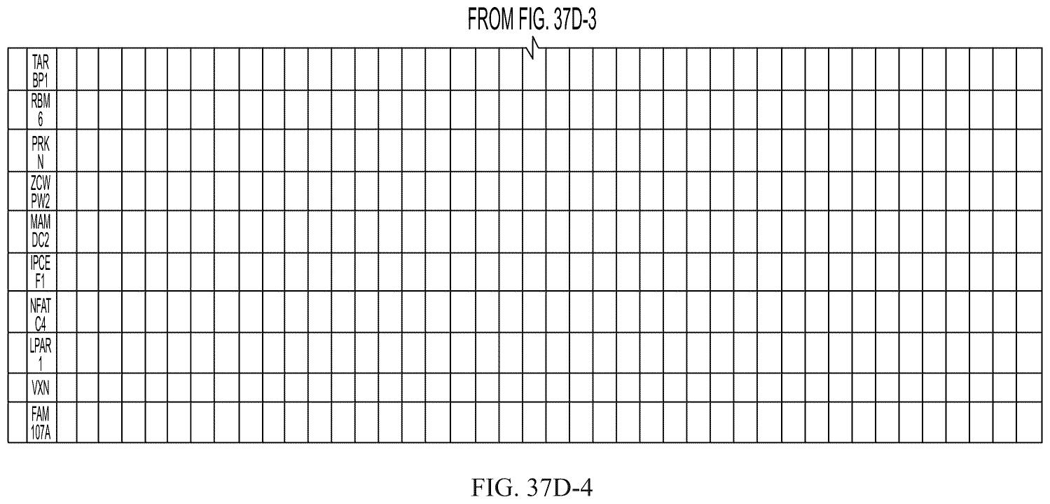

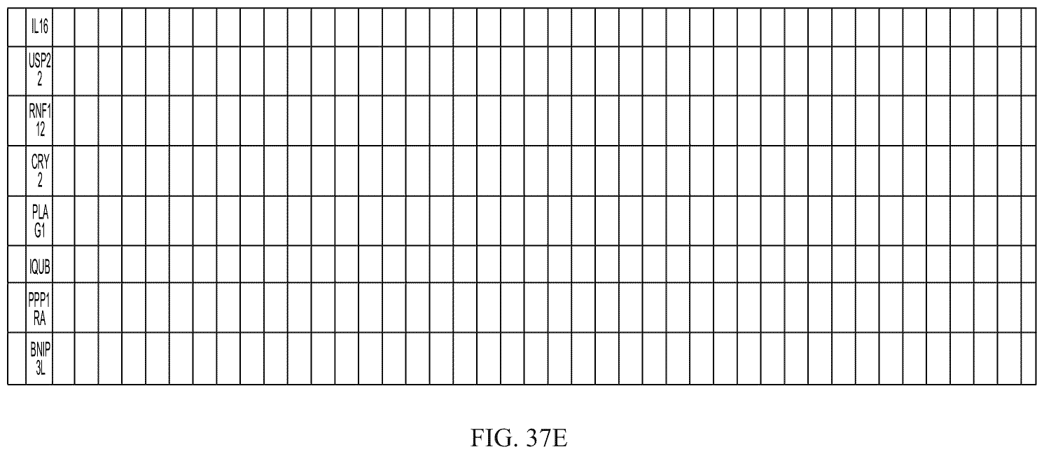

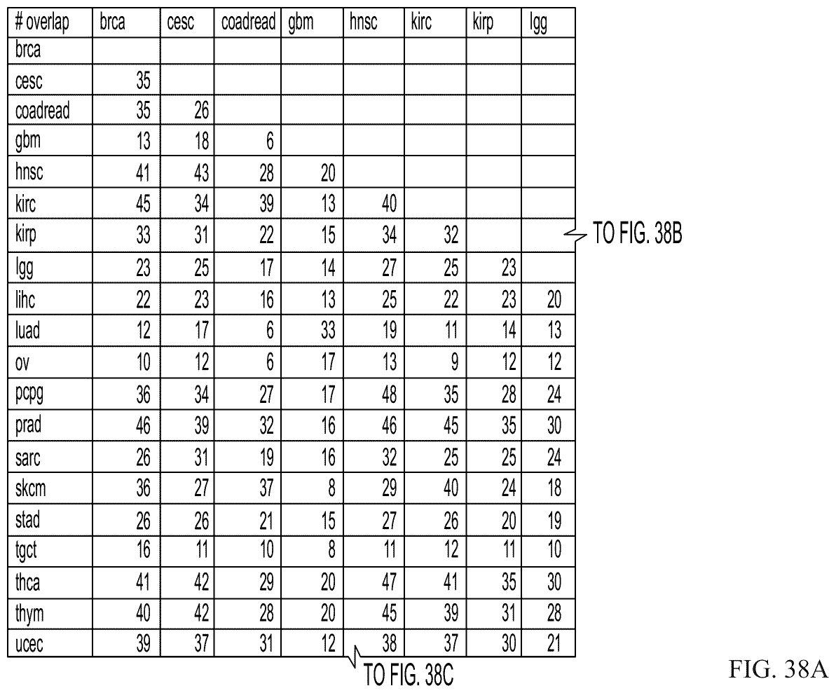

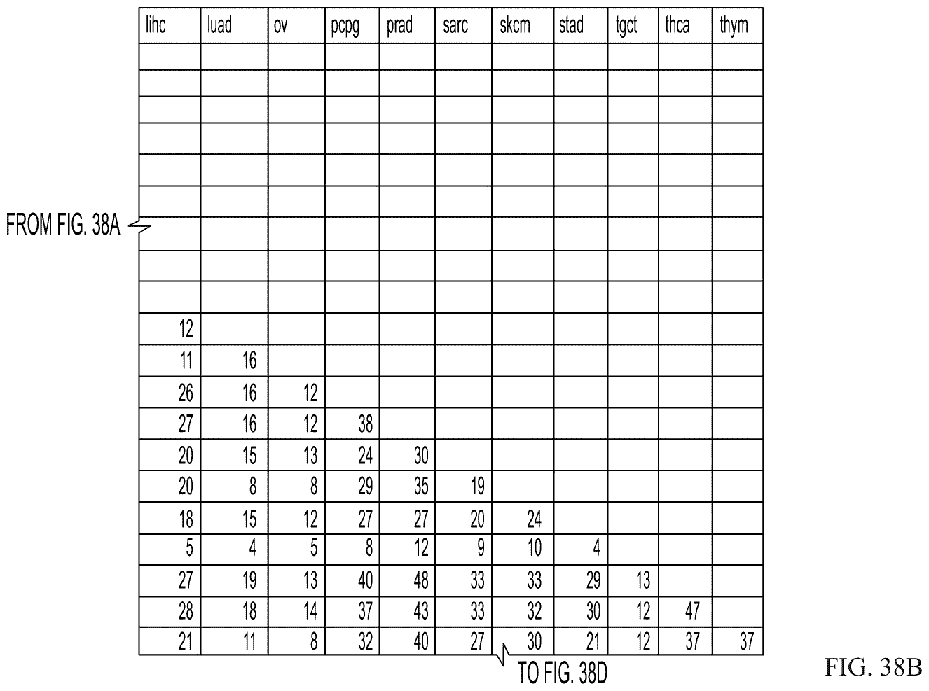

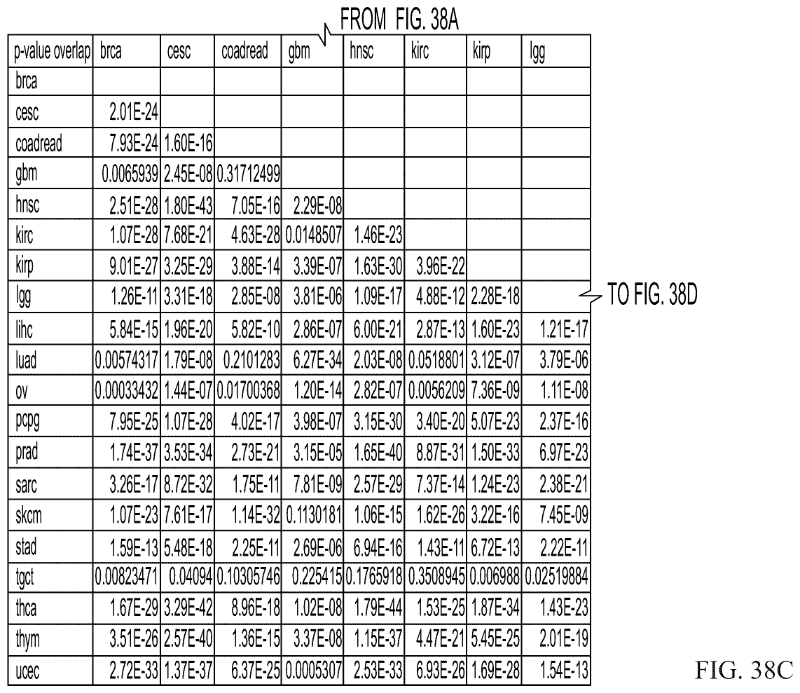

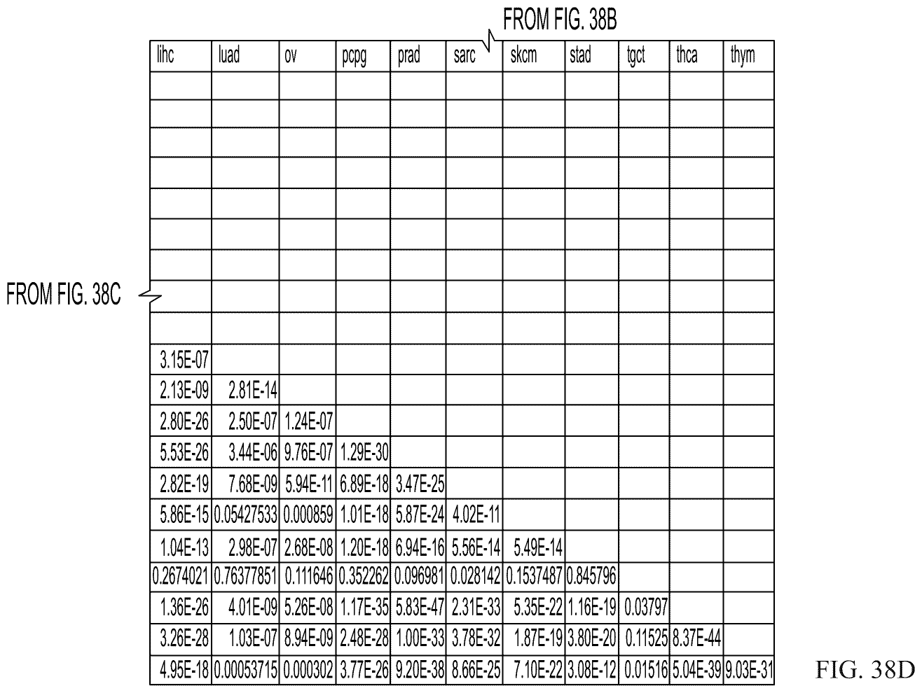

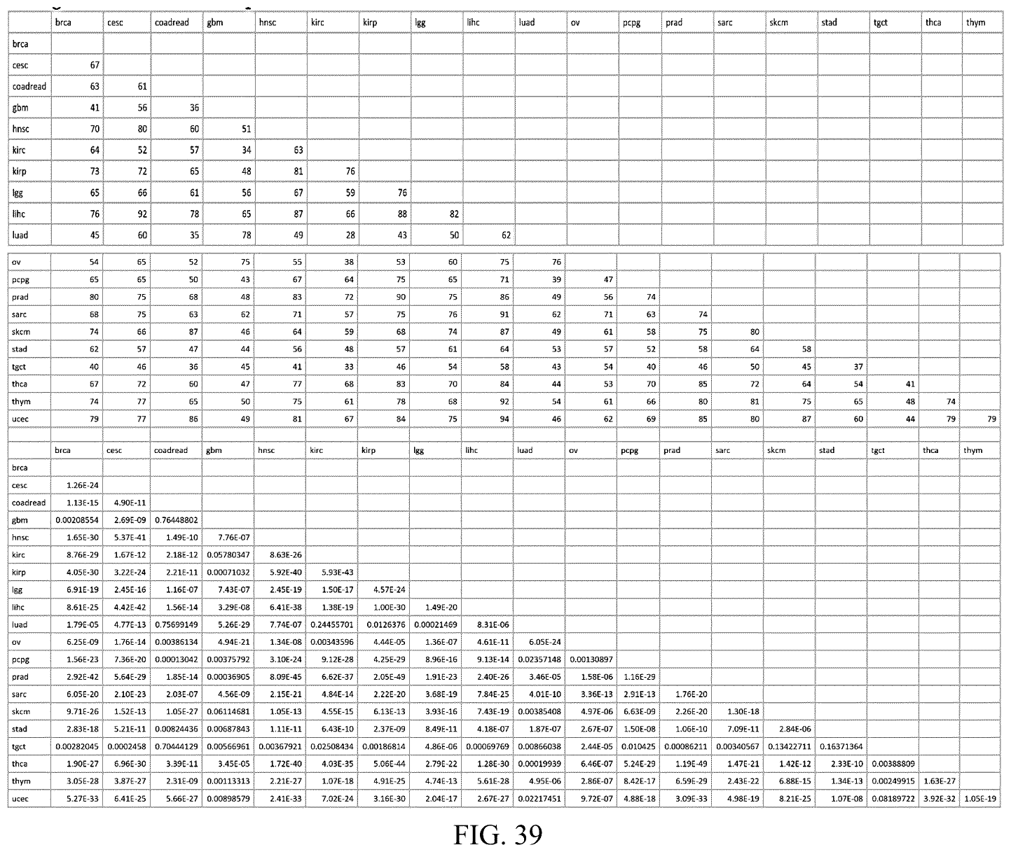

As an additional method to investigate whether speckles vary among individuals of cancer types beyond ccRCC, speckle protein gene expression patterns for 19 additional cancer types was assessed using RNA-seq data from The Cancer Genome Atlas (downloaded through cBioPortal in 2018). For each cancer type, the speckle protein genes that contribute the most to patient variation were extracted by taking the speckle protein genes with the highest rotation values in principle component 1 from principal component analysis (FIGS. 37A-37E). Similar to ccRCC, this analysis revealed two reciprocally-expressed groups of speckle protein genes among tumor samples. Comparing the groups of genes between cancer types, a high degree of overlap from cancer type to cancer type was observed. The two speckle protein groups in each cancer type were therefore assigned to speckle Signature I or Signature II, defining Signature II as the speckle protein group containing the protein SON, and calculated the significance of speckle protein gene overlap for each pairwise comparison of the 20 cancer types, including ccRCC (called kirc in TCGA data). In 19 of the 20 cancer types, a substantial overlap was found between the different cancer types, both in the speckle protein genes from speckle Signature I (FIGS. 38A-38D), and those from speckle Signature II (FIG. 39). This finding demonstrates that the two speckle signatures are reproducibly found across many cancer types. This discovery enabled the definition of a set of 18 speckle protein genes that were found in speckle Signature I or speckle Signature II in nearly all cancer types (at least 16 of 20), constituting a minimal speckle signature that is sufficient to separate patients into the speckle signature groups.

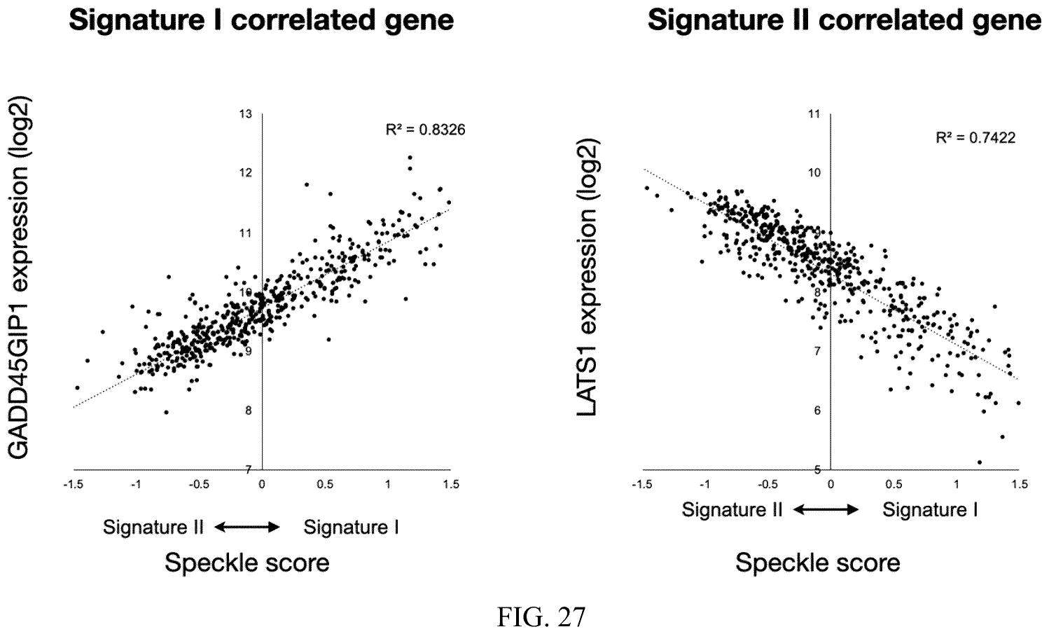

The finding that the speckle signature is consistent across cancer types also allowed for the identification of what genes in the genome are highly correlated with speckle signature irrespective of the cancer type. This involved assigning each patient a speckle score based on speckle protein gene expression (see “Using speckle signature as a prognostic indicator”), and calculating the Spearman's correlation coefficient between the speckle score and gene expression of every gene in the genome. This analysis revealed the most highly correlated genes with the speckle signature, including GADD45GIP1 and LATS1 (FIG. 27). As the speckle prognostic portions of the present invention are developed further, these observations will be of particular use to define a minimal set of genes that is capable of separating patients by speckle signature groups.

### Speckle Signature Predicts Patient Outcomes Depending on Mutation Status.

Separating patients by speckle signature did not reveal any other cancer types other than ccRCC (kirc) among the TCGA PANCAN dataset where speckle signature was predictive of overall patient survival. Without wishing to be bound by theory, it was hypothesized that this was because ccRCC has more homogenous etiologies as compared to other cancer types, with nearly all patients displaying hyperactive HIF2A. Therefore, to obtain an indication of whether speckle signature predicts patient outcomes in particular cancer subclasses, each cancer type was separated based on the top mutated genes within the cancer. In doing so, five additional cases were identified where speckle signature predicted or informed patient outcomes, detailed below. Notably not all cancer subtypes have been exhaustively analyzed. Hence, there are likely many more circumstances where speckle signature predicts patient outcomes.

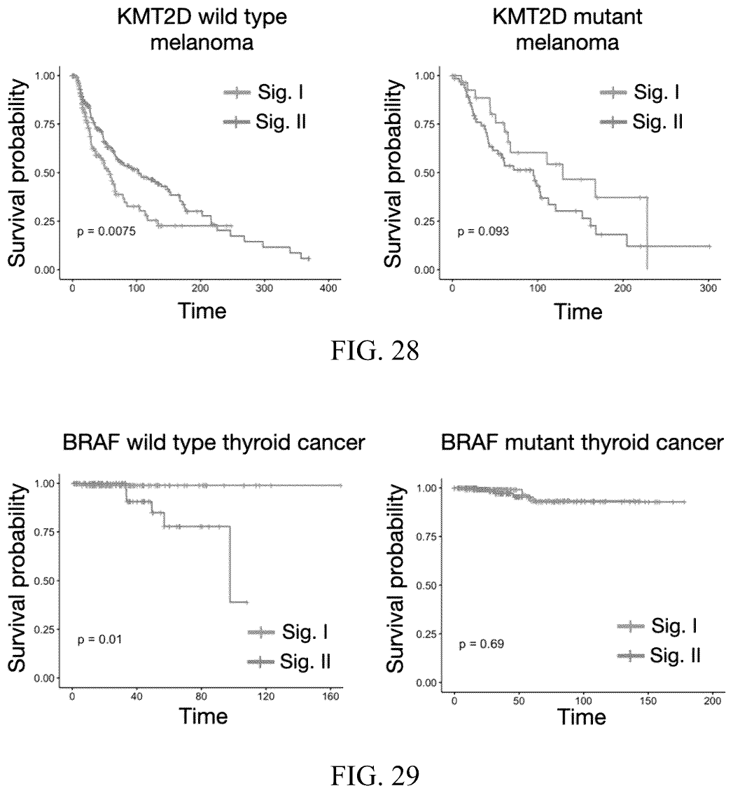

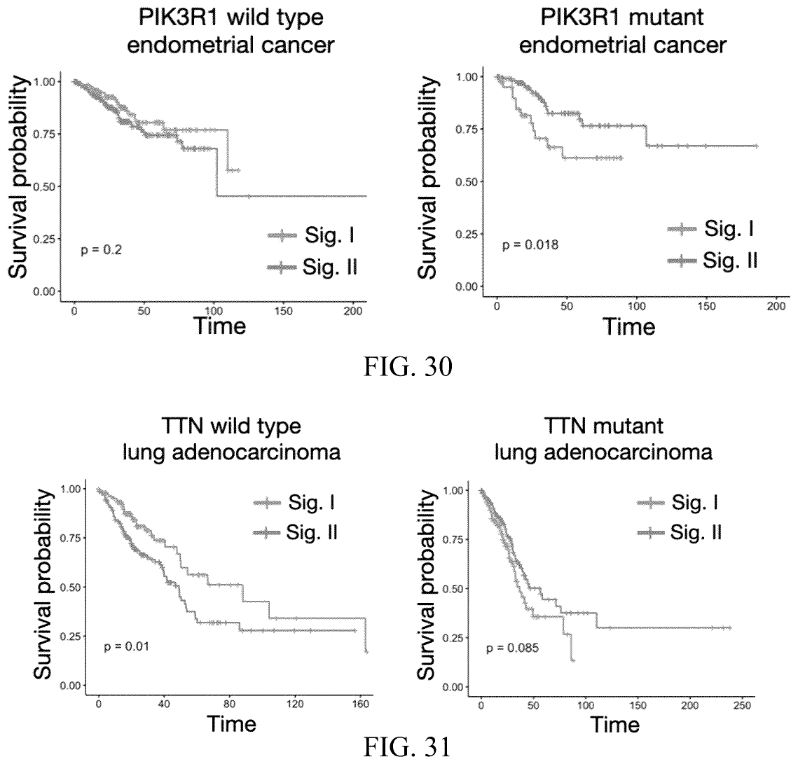

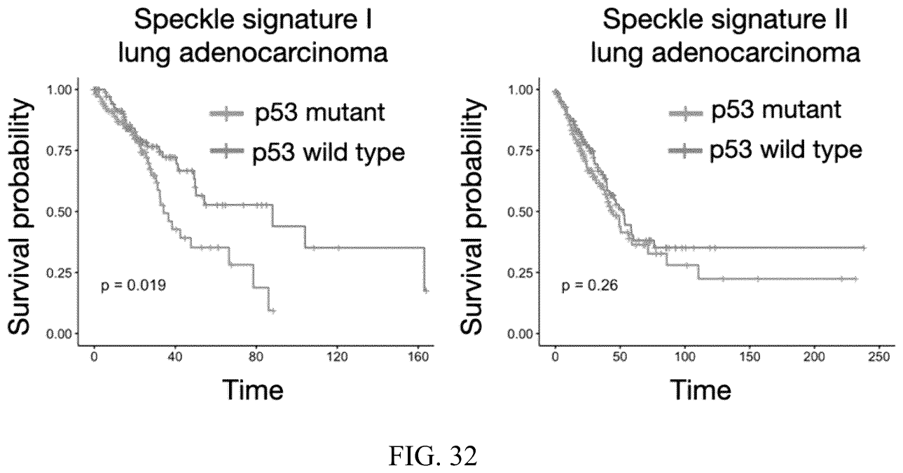

These studies found that speckle signature predicts patient outcomes in the following cases:
1. In KMT2D wild type melanoma, speckle Signature I is associated with poorer survival (p<0.01), while in KMT2D mutant melanoma, speckle Signature II trends towards poorer survival (p<0.1) (FIG. 28). Note that KMT2D is an STM-containing co-activator. Hence, compositions and methods that target the speckle associating abilities or that target the speckle signature may be effective.
2. In BRAF wild type thyroid cancer, speckle Signature II is associated with poorer survival (p<0.01; FIG. 29). This case together with TTN wild type lung adenocarcinoma, below, provides an application for shifting the speckle signature from Signature I to Signature II.
3. In PIK3R1 mutant endometrial cancer, speckle Signature I is associated with poorer survival (p<0.05; FIG. 30). This poor prognosis of speckle Signature I is similar to the ccRCC example, with similar methods potentially applicable
4. In TTN wild type lung adenocarcinoma, speckle Signature II is associated with poorer survival (p<0.05), while in TTN mutant lung adenocarcinoma, speckle Signature I trends towards poorer survival (p<0.1) (FIG. 31). TTN is a speckle target motif-containing protein. However, it is also highly correlated with mutational burden in cancer. This is particularly important for lung adenocarcinoma, which separates into non-smokers with low mutational burden and smokers with high mutational burden. Thus, it is possible that our findings here reflect differences of patient survival in the subgroup non-smoker lung adenocarcinoma patients. This line of reasoning provides rationale for investigating the importance of speckle signature for cancer subtypes defined by variables other than the top mutated genes.
5. Lung adenocarcinoma with mutant p53 has worse prognosis than those with wild type p53 specifically in patients with speckle Signature I (FIG. 32).

In total, the identification of several subtypes of cancer where speckle signature is predictive of patient survival indicates a high potential for speckle targeting therapies to become therapeutic strategies. Meanwhile, the speckle signature provides a new prognostic method for identifying high-risk patients who may benefit from particular treatment options.

### Example 5: Nuclear Speckle Positioning Predicts Patient Prognosis in Clear Cell Renal Cell Carcinoma

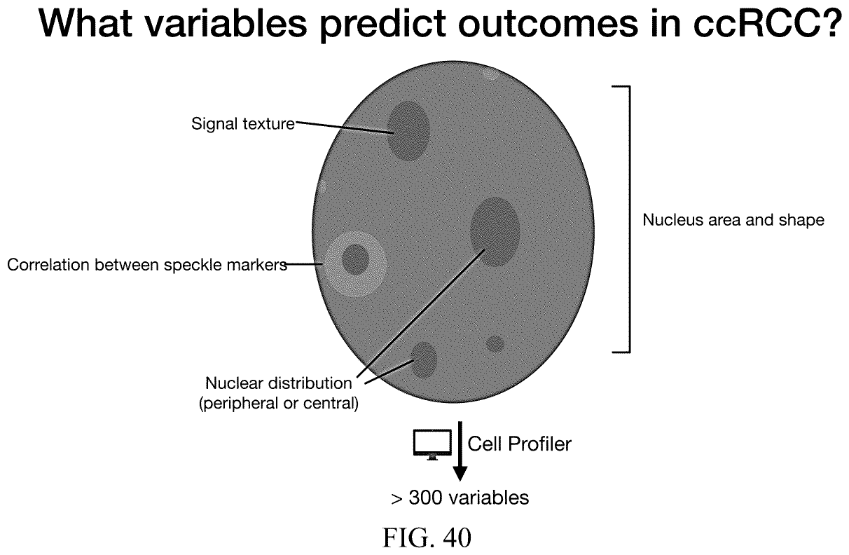

The data presented in the present disclosure demonstrates that positioning of genes in relation to nuclear speckles is a novel mechanism of gene regulation utilized by transcription factors (ie. p53 in Alexander et al., 2021 and HIF2A in the present disclosure). Additionally, these data demonstrated that nuclear speckle expression patterns, as assessed in RNA-seq data, are predictive of patient survival in VHL mutant clear cell renal cell carcinoma, KMT2D wild type melanoma, BRAF mutant thyroid cancer, PIK3KR1 mutant endometrial cancer, and TTN wild type lung adenocarcinoma (see previous discloser Example 4). In addition, speckle expression patterns informed survival prediction in lung adenocarcinoma separated by p53 mutation status. Based on these data, and without wishing to be bound by theory, it was hypothesized that nuclear speckles may serve as a prognostic indicator depending on the underlying transcriptional and mutation cancer dependencies. Particularly in clear cell renal cell carcinoma, which is characterized by hyperactivation of the speckle-targeting transcription factor HIF2A, which involves inactivating mutations of the VHL protein. Previous RNA-based estimations of nuclear speckle phenotypes can be limited because they 1) were an indirect assessment of nuclear speckle phenotypes, and 2) lacked scalability to enable large-scale application of a prognostic method. In this example, these limitations were addressed by applying an immunofluorescence-based protocol to directly visualize nuclear speckles in FFPE tissue sections, which are routinely collected for pathology in the clinic. It was unexpectedly discovered that the radial positioning of speckles within tumor cell nuclei was highly predictive of survival in clear cell renal cell carcinoma, providing a robust immuno-based imaging assay to classify high-risk patients based on their nuclear speckle phenotype (see FIG. 40).

Radial Positioning of Nuclear Speckles within the Cell Nucleus Predicts ccRCC Patient Outcomes

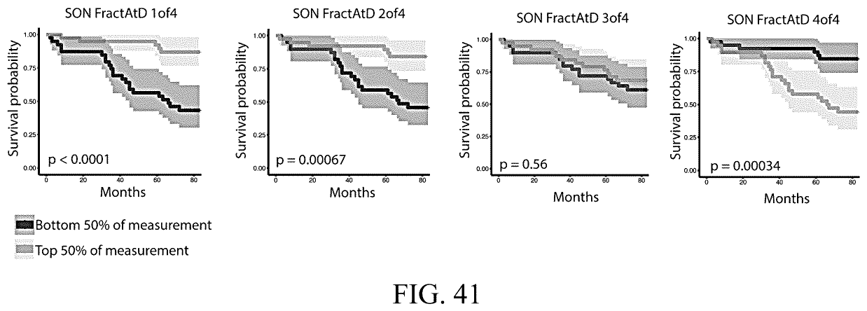

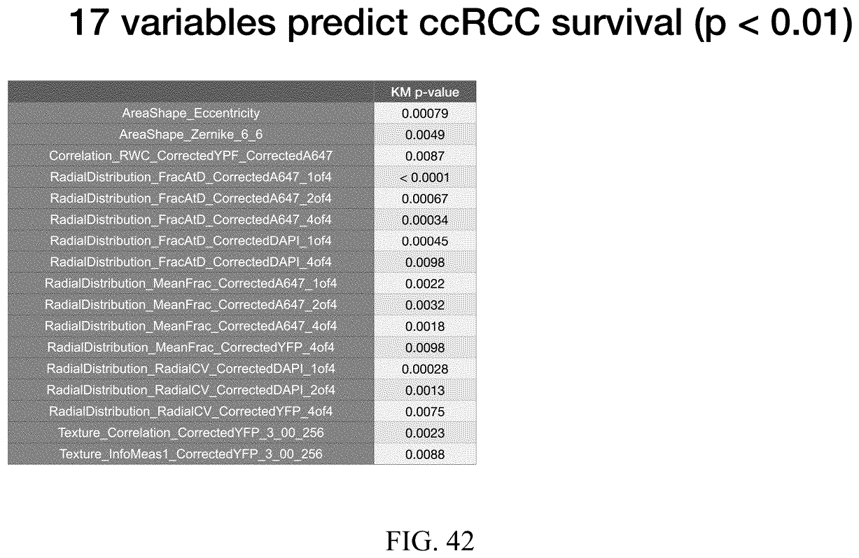

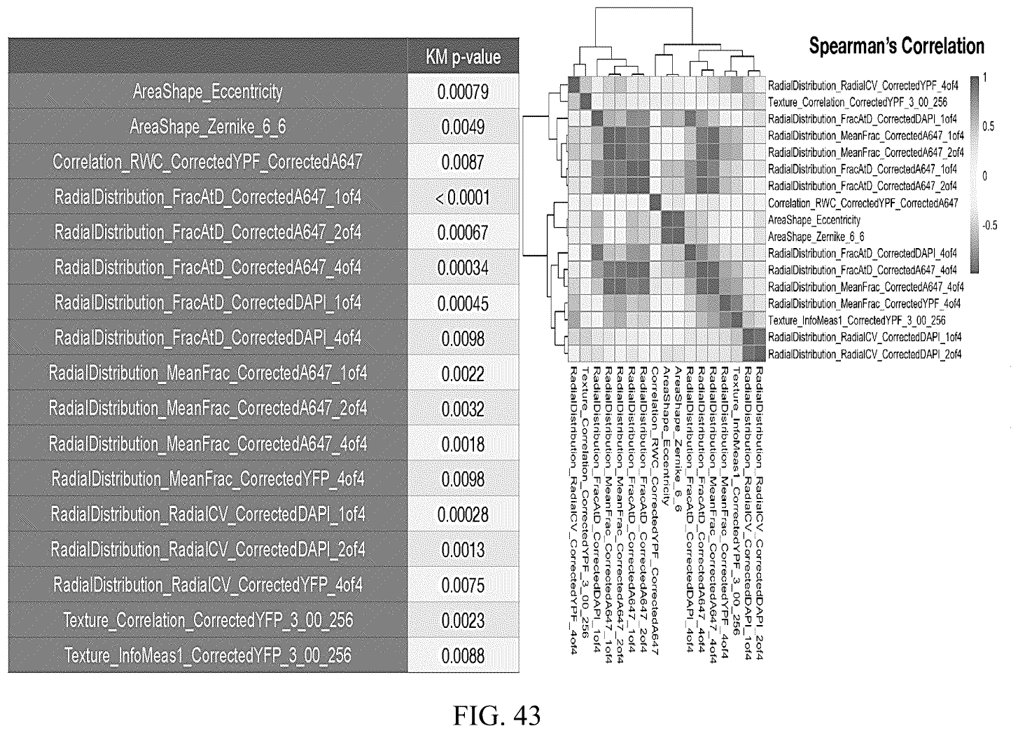

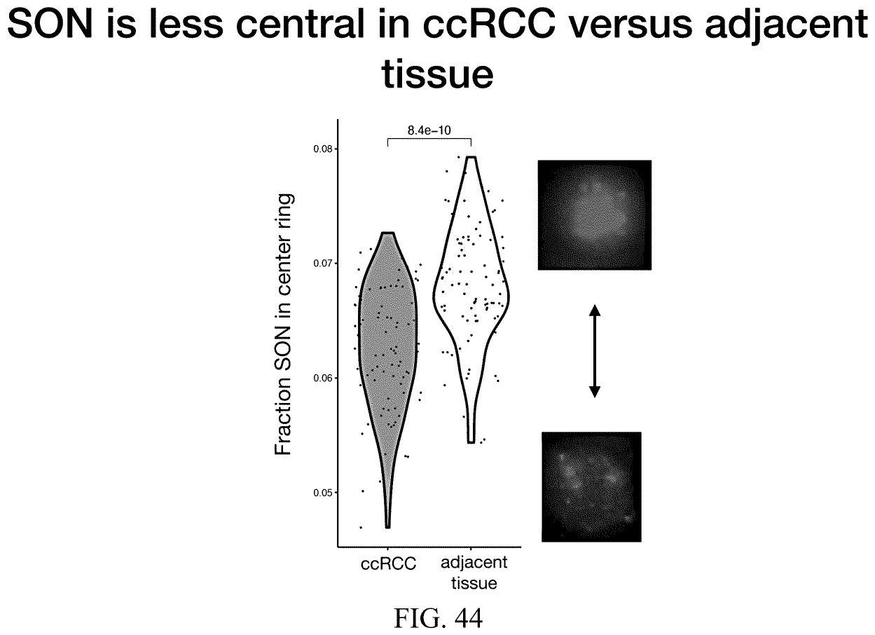

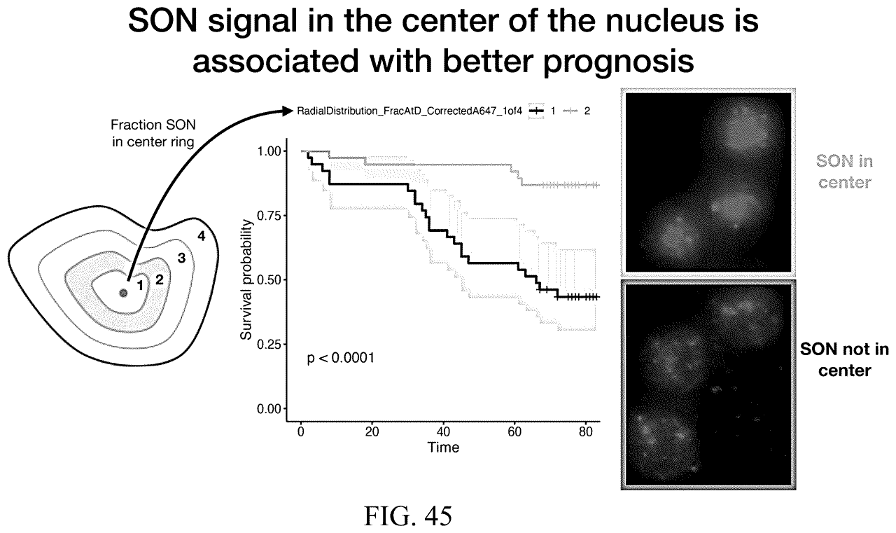

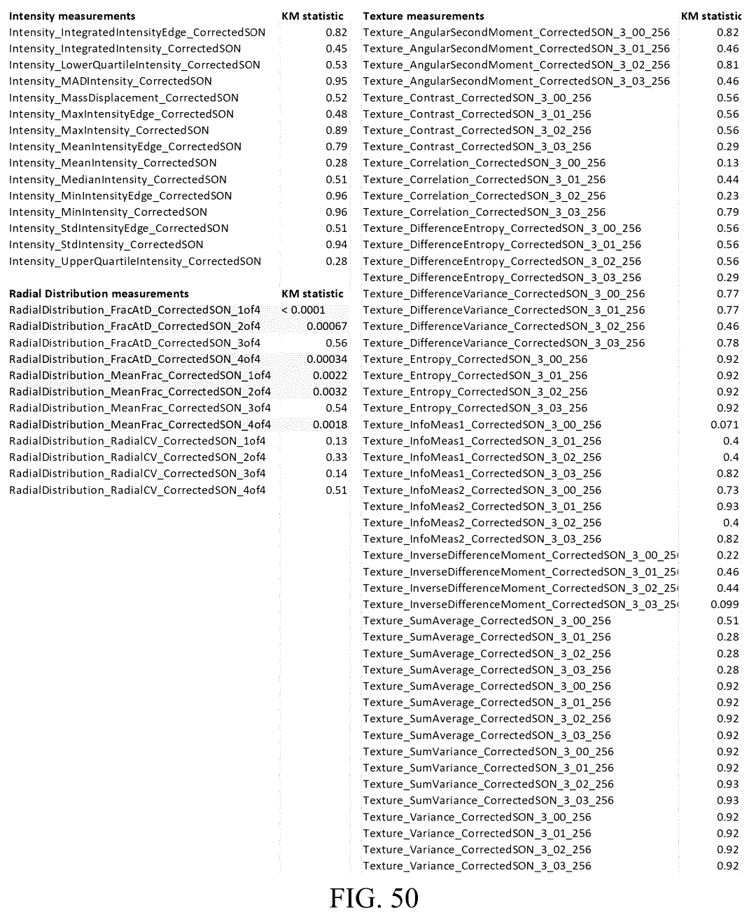

To determine whether nuclear speckle phenotypes predict patient outcomes in clear cell renal cell carcinoma (ccRCC), a tissue microarray containing 90 ccRCC tissue samples and 90 matched adjacent tissues was obtained, of which 77 had associated patient survival data. Immunofluorescence of the well-established speckle marker protein SON was then employed, together with DAPI staining, followed by imaging the entirety of each sample at 20× magnification. The correlation between speckle phenotypes and patient outcomes was then assessed. From each sample, per-nucleus SON intensity, texture, and radial distribution measurements was assessed, for a total of 79 SON-related measurements, which were used to calculate Kaplan Meier statistics by splitting the patient population into the top and bottom half based on the median value of all nuclei within the sample. Using this method, it was found that none of the intensity or texture measurements of SON immunofluorescence significantly (p<0.01) predicted ccRCC patient survival (FIG. 50). In contrast, several measurements of SON radial positioning were significantly correlated with survival (FIG. 50 and FIG. 41).

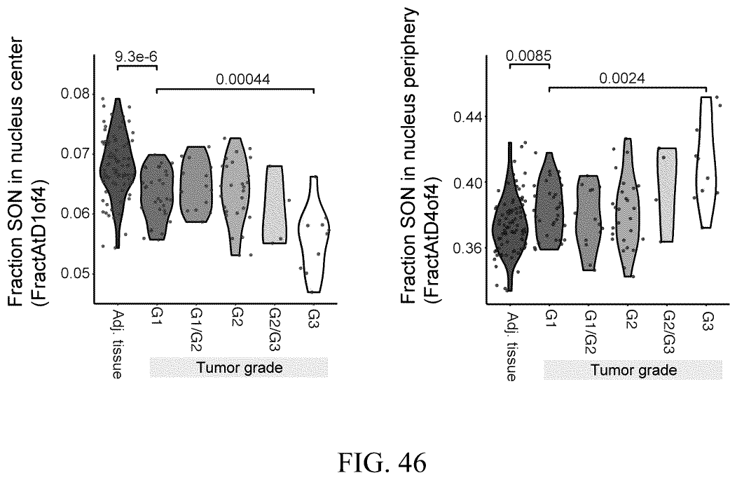

Radial distribution measurements were performed by binning the nucleus into four bins, the innermost (bin 1 of 4) to the outermost bin (bin 4 of 4), and calculating the fraction of signal (FractAtD), the mean fractional intensity (MeanFrac), or the coefficient of variation (radialCV) of SON signal within each bin. Specifically, it was found that ccRCC patients with high fraction of SON signal at the center of the nucleus (FractAtD 1 of 4) displayed favorable survival, while ccRCC patients with low fraction of SON signal at the center of the nucleus displayed unfavorable survival (FIG. 41, left; p<0.0001). Consistently, patients with high fraction of SON at the periphery of the nucleus (FractAtD 4of4) showed less favorable outcomes as compared to patients with low fraction of SON at the periphery of the nucleus (FIG. 41, right; p=0.00034). Examples of ccRCC tumor samples with high central SON or high peripheral SON are shown in FIG. 46. These findings demonstrate that central positioning of speckles within the cell nucleus is associated with favorable outcomes in ccRCC, while peripheral positioning of speckles within the cell nucleus is associated with poor outcomes.

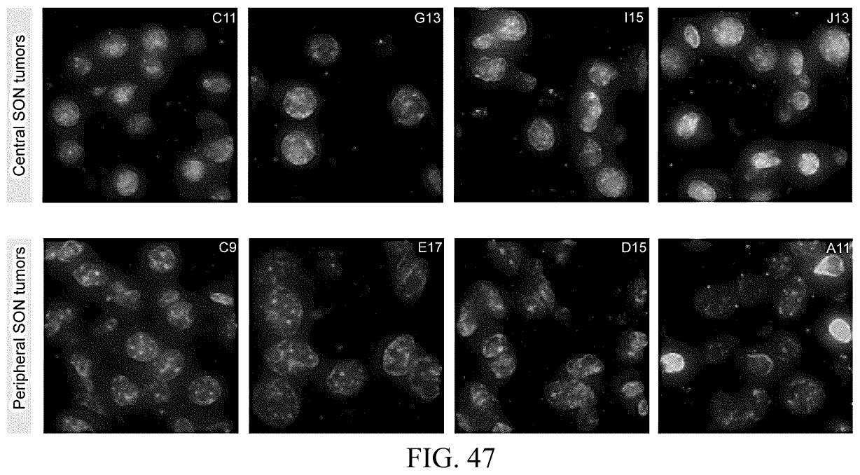

Another study was performed which directly compared RNA- and imaging-based measurements of speckle phenotype in the same cohort of samples, with the hypothesis that samples with lower central SON would correspond to Signature I speckle protein gene expression. Thus, clinical ccRCC tumor and tumor-adjacent samples were obtained and divided in order to perform both RNA-seq and FFPE SON immunofluorescence (FIG. 52A, left schematic), including three tumor-adjacent primary tubule renal epithelium samples, three primary human ccRCC tumors, and four patient-derived mouse xenograft ccRCC tumors derived from the human individual. First, speckle protein gene expression scores were calculated via RNA-seq as previously described herein, and then the same tissue/tumor was imaged. Primary renal tubule epithelial samples (normal adjacent) displayed Signature II speckle scores and high central SON by imaging (FIG. 52A; dark, lower-right points; N—normal primary renal tubule epithelium), while two primary ccRCC tumors also showed Signature II speckle scores with high central SON (FIG. 52A; light points; T—primary tumor); the remaining primary ccRCC tumor and all four patient-derived xenograft samples showed the opposite Signature I speckle scores, with corresponding low central SON (FIG. 52A; see yellow primary tumor point and dark, upper left xenograft points (Tx) on upper left portion of graph). These direct comparisons indicate that speckle Signature I manifests as more spread out/less central speckles, associated with worse ccRCC survival (e.g. FIG. 47, top), while, in contrast, speckle Signature II manifests as central larger speckles associated with better ccRCC survival (e.g., FIG. 47, bottom). Therefore, these data directly link RNA-seq and imaging-based speckle phenotypes, demonstrating that they may be used interchangeably, adding to potential therapeutic relevance.

Speckle Signature Correlates with ccRCC Tumor/Patient Drug Response

Without wishing to be bound by theory, it is envisioned that having both RNA- and imaging-based methods for measuring speckle phenotypes will assist in the development of cancer- and patient-specific treatment strategies. As a proof-of-concept for a potential use of nuclear speckle phenotyping, a series of studies was undertaken in which speckle signature was correlated with tumor/patient drug response in available data from a human clinical trial and patient-derived mouse xenograft studies.

Comparing RNA speckle signature between xenograft tumors that were sensitive or resistant to the PT2399 HIF-2a inhibitor, it was found that ˜75% of Signature I tumors were sensitive to PT2399, while only ˜30% of Signature II tumors were sensitive (FIG. 52B), suggesting that Signature I tumors were more likely to be sensitive to HIF-2a inhibition. As a second potential application, it was found that in a ccRCC clinical trial comprised of mTOR inhibitor (everolimus) and PD1 inhibitor (nivolumab) arms, Signature I patients did not differ in overall survival between the two treatment groups, while Signature II patients had higher overall survival probability when treated with nivolumab compared to everolimus (FIG. 52C). Thus, contrasting with HIF-2a inhibition, PD1 inhibition may have a greater impact in individuals with Signature II tumors. Without wishing to be bound by theory, these findings suggest differential drug sensitivities depending on tumor speckle signatures, emphasizing the need and potential utility of evaluating how speckles relate to tumor/patient drug responses.

High Grade ccRCC have Less Central and More Peripheral Nuclear Positioning of Speckles

Studies next compared the radial positioning of SON signal between matched adjacent tissue and ccRCC tissue separated by tumor grade. Compared to adjacent tissues, ccRCC tumor samples had less central SON (FIG. 47, left) and more peripheral SON (FIG. 47, right). The fraction of SON signal in the center of the nucleus also decreased with tumor grade (compare G1 with G3). Reciprocally the fraction of SON signal in the periphery of the nucleus increased with tumor grade. Hence nuclear speckle positioning becomes dysregulated in ccRCC compared to adjacent tissue, and this dysregulation becomes more severe in later grade tumors.

Radial Positioning of Speckles within the Nucleus is Predictive of ccRCC Survival in Low Grade ccRCC

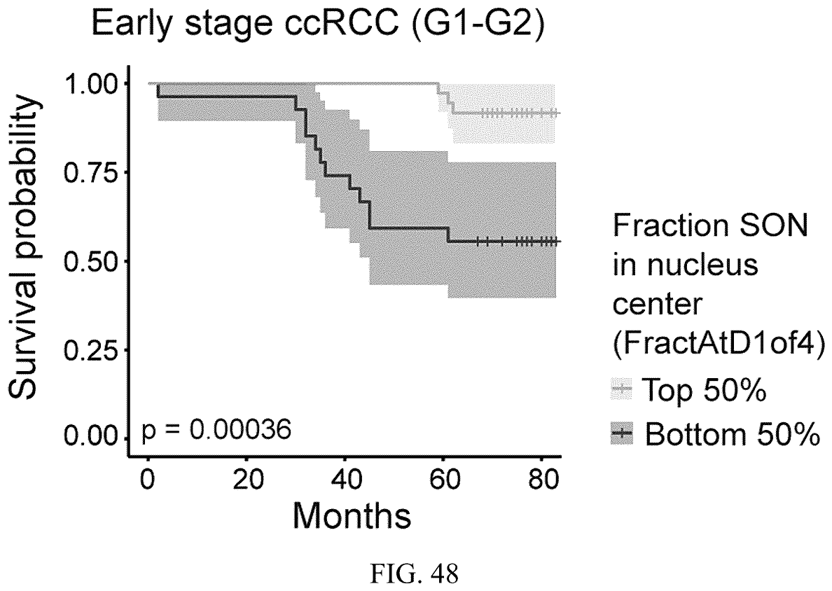

While later grade ccRCC displayed the most dramatic differences in radial distribution of nuclear speckles as compared to adjacent tissue, early stage ccRCC displayed a distribution of speckle positioning. To determine whether speckle positioning within early grade tumors is predictive of survival, survival analysis was performed including only Grade 1 and Grade 2 tumors (G1, G1/G2, and G2). It was found that nuclear speckle radial distribution still predicted patient outcomes in lower grade ccRCC (FIG. 48). These results demonstrate that the poor outcome of tumors with low central speckle positioning can be predicted at early stage ccRCC. This finding is critical because it enables classification of patient risk groups at early clinical stages based on nuclear speckle phenotypes.

### Stratification of High-Risk Nuclear Speckle Radial Positioning

To examine whether a particular nuclear speckle signal radial positioning cutoff could be used to stratify high-risk ccRCC patients, studies then evaluated Kaplan Meier statistics using different values for the fraction of SON in the center of the nucleus (FractAtD1of4), which was most predictive of patient outcomes when patients were split into the top and bottom 50% based on this measurement. It was found that splitting patients at a SON FractAtD1of4 of 0.0615 had the most significant Kaplan Meier p-value for early stage ccRCC (p=0.00012), and thus may serve as a reference point for risk assessment. Ten percent of the matched adjacent tissue samples (9 of 90) and 44.4% of the ccRCC samples (40 of 90) were found to be below this reference value. These metrics provide guidance for setting thresholds for classifying high-risk ccRCC patients.

Additional Predictors of ccRCC Patient Outcomes

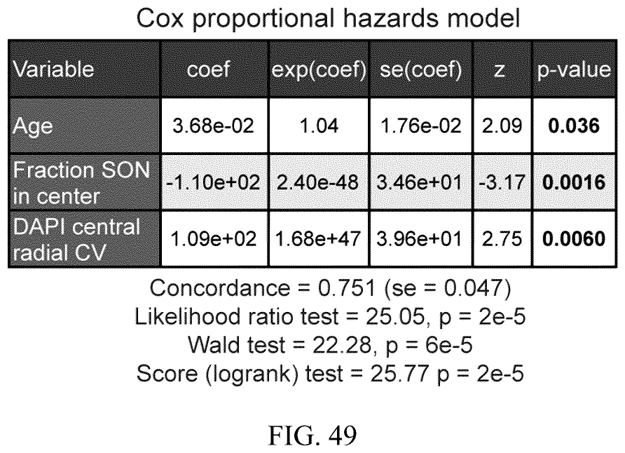

To quantify the effect of nuclear speckle radial positioning, and to assess the impact of different variables on ccRCC outcome predictions, a Cox proportional hazards model was generated. It was found that subject age, radial distribution of SON signal (FractAtD1of4 for SON), and the coefficient of variation for the central DAPI radial fraction (RadialCV1of4 for DAPI) were each separately predictive of ccRCC patient outcomes, and together were highly predictive of ccRCC patient outcomes as assessed by the model (FIG. 49). These results demonstrate that SON radial positioning is predictive of ccRCC outcomes even when subject age is accounted for, and illustrate a method to refine patient risk classification by combining information from speckle positioning with the simultaneously-collected DNA staining data.

### Speckle Signature I Tumors are Enriched in Oxidative Phosphorylation and Ribosome Pathways

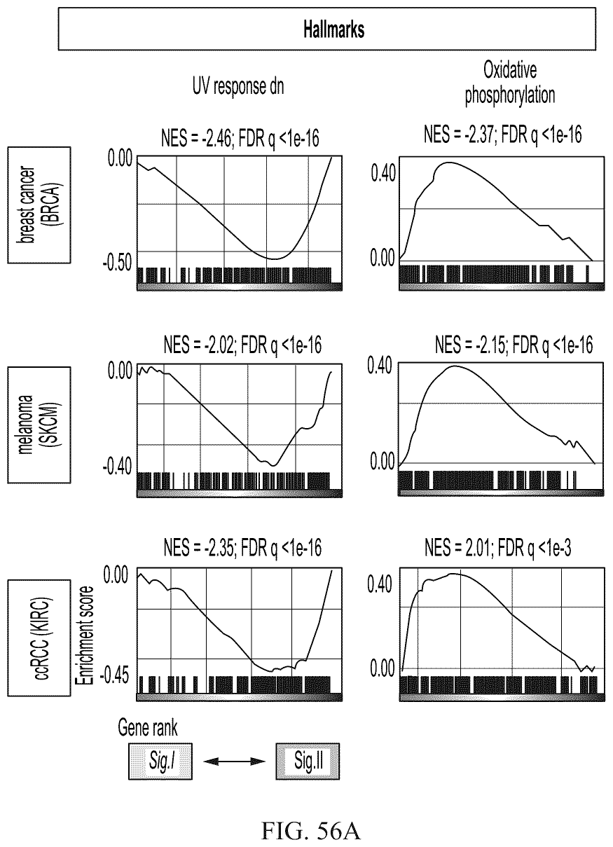

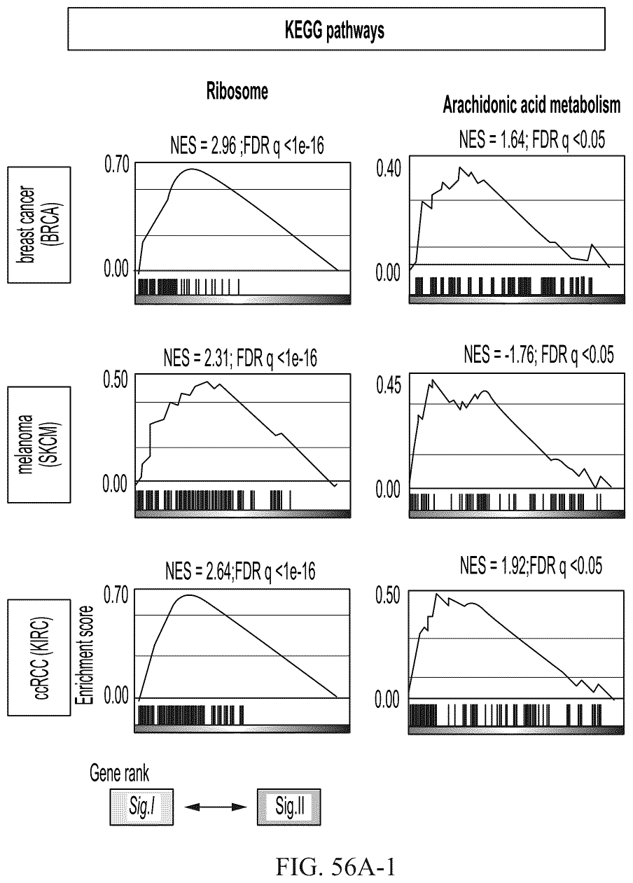

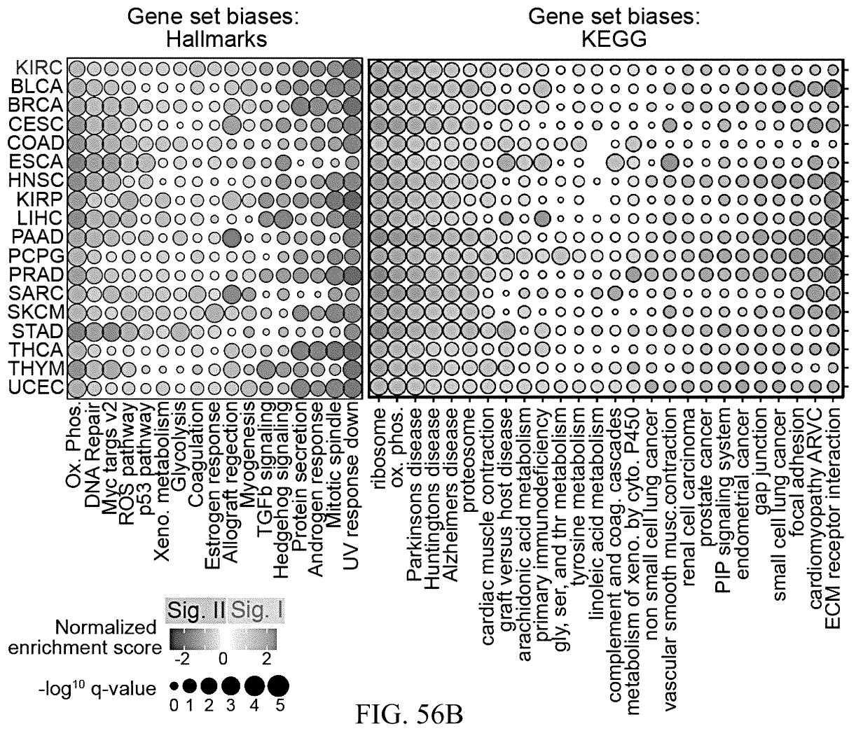

The speckle signature, while present in many cancers, was particularly predictive of survival in ccRCC (see FIG. 55). Given the findings presented herein that HIF-2a regulates DNA-speckle association, we hypothesized that HIF-2a combines with speckle phenotypes, resulting in poor ccRCC outcomes. To broadly understand the consequences of cancer speckle signature, attention was shifted to deeper analysis of gene expression differences between speckle signature patient groups in TCGA data. TCGA samples were divided into Signature I and Signature II groups using the top and bottom 25% of sample speckle scores (from FIG. 55; Signature I—top 25%, Signature II—bottom 25%), calculated gene expression fold changes, and used Gene Set Enrichment Analysis (GSEA) to identify which biological pathways were differential between the two patient groups. We found striking enrichment of “Oxidative phosphorylation” and “Ribosome” in the Signature I group among all cancer types, including ccRCC (KIRC) (FIGS. 56A-56B). Hence, across many cancer types, speckle Signature I correlates with increased oxidative phosphorylation and ribosomal pathways, suggesting that speckle Signature I tumors, which reflect the aberrant cancer speckle signature, may exist in a “hyper-productive” state with enhanced metabolic and protein production capacity. Based on these findings, and without wishing to be bound by theory, it is hypothesised that while speckle signature does not correlate with overall survival in all cancer types, it may broadly predict responses to therapy, particularly therapies that target metabolism and protein production pathways.

Methods Details for this Example

Antibody staining of FFPE tissue sections. The tissue array, HKID-CRC180SUR-01 contain 90 ccRCC samples and 90 matched adjacent 5 micron tissue sections with associated survival and grade data was obtained from USBioMax and stained for nuclear speckles using the following method. The slide was baked for 2 hours at 60° C. to help tissues sections adhere to the slide and deparaffinized and re-hydrated with 3×5 minute washes in Xylenes, 2×10 minute washes each in 100%, 95%, 80%, 70%, and 50% ethanol, 2×5 minute washes in deionized water. Antigen retrieval was performed in 1×HIER antigen retrieval buffer (ab208572) for 5 minutes in a pressure cooker. The slide was washed 2×5 minutes in deionized water, then blocked for 90 minutes in 10% goat serum in PBS with 0.2% Triton X-100. Primary antibody (SON; ab121759) was applied at a 1:100 dilution in 1% goat serum in PBS with 0.2% Triton X-100 and incubated overnight in a humidified chamber. The slide was washed 2×10 minutes in 1% goat serum in PBS with 0.2% Triton X-100, and the slide was incubated in secondary antibody (ThermoFisher A-21245) for one hour at room temperature. The slide was washed for 10 minutes in 1% goat serum in PBS with 0.2% Triton X-100, then DAPI stained at a final concentration of 0.2 ug/mL for 10 minutes in 1% goat serum in PBS with 0.2% Triton X-100, then washed 2×10 minutes in 1% goat serum in PBS with 0.2% Triton X-100. Excess liquid was drained from the slide, mounting media (20 mM Tris pH 8.0, 0.5% N-propyl gallate, 90% Glycerol) was added to cover tissue sections, a coverslip was placed over the mounting media, and the coverslip was sealed with nail polish.

Imaging. Tissue sections were scanned at 20× magnification on a wide-field Nikon 2iE microscope (objective lens: CFI60 Plan Apochromat Lambda 20× Objective Lens, N.A. 0.75, W.D. 1.0 mm, F.O.V. 25 mm, DIC, Spring Loaded) with 7 optical sections, imaging over 2000 nuclei per sample, and covering the entirety of the tissue section.

Analysis. Maximum Z projections were made using CellProfiler with the module “MakeProjection” with the Type of projection set to “Maximum”, and saved using the module “SaveImages”. Using the resultant maximum projections as input, the following steps were performed in CellProfiler: uneven illumination was calculated and corrected using modules “CorrectIlluminationCalculate” and “CorrectIlluminationApply”, and nuclei were segmented using “IdentifyPrimaryObjects” on the DAPI signal. Per-nucleus intensity, radial distribution, and texture measurements were performed using the CellProfiler modules “MeasureObjectIntensity”, “MeasureObjectlntensityDistribution”, and “MeasureTexture” applied to the aforementioned nuclei objects. These per-nuclei measurements were performed for each of the 90 ccRCC and 90 matched adjacent tissues and exported. Next, the per-sample medians were calculated for each per-nucleus measurement, and Kaplan Meier statistics were performed by splitting ccRCC subjects based on the top and bottom 50% based on these median measurements.

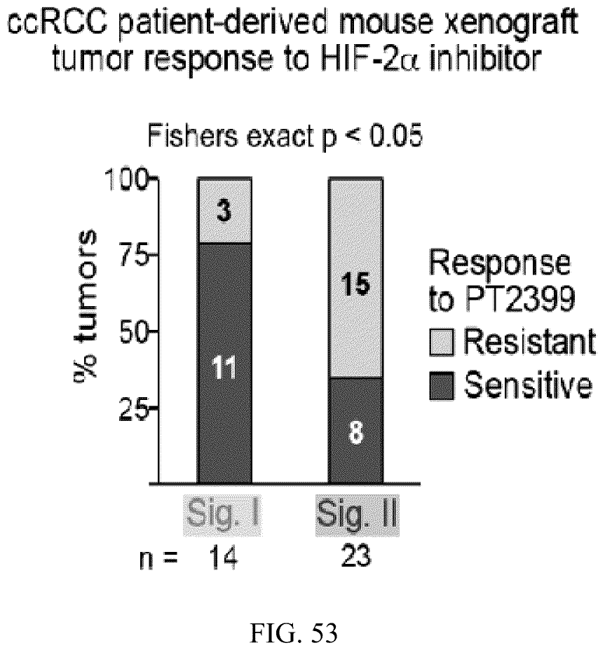

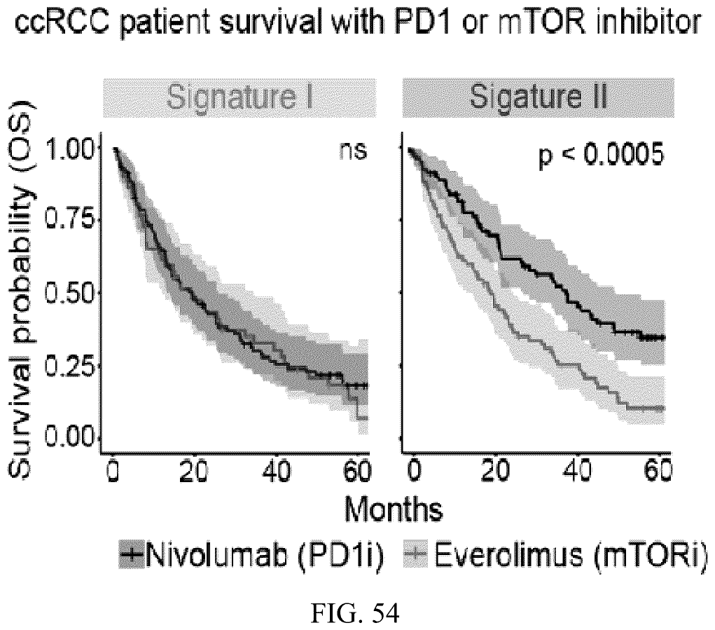

Methods for determining speckle signature and TCGA survival analysis. Four-hundred and forty-six protein genes annotated as “Enhanced”, “Supported”, or “Approved” for subcellular localization within nuclear speckles were identified in The Human Protein Atlas and their upper-quartile normalized RNA expression was extracted from the 30 PanCan TCGA projects that had greater than 50 samples. Principal Component Analysis was then performed on these 446 speckle protein genes. In doing so, each speckle protein gene was assigned a weight (called rotation in the analysis) that was used in the analysis to separate tumor sample along the first Principal Component (PC1). The absolute value of a speckle protein gene PC1 weight thus estimates the contribution of each speckle protein gene to patient variation and the PC1 weight sign, positive or negative, reflects genes that have opposite expression patterns to one another. To compare speckle protein gene expression contributions to patient variation between cancer types, the pairwise Pearson's correlation coefficients of the speckle protein PC1 weights were used. In order to obtain a set of speckle protein genes that consistently contributed to patient variation in many cancer types, the rotation signs were flipped so that the speckle protein gene, SON, was always assigned a negative weight. The speckle protein genes that had consistently signed rotations were then extracted across 22 cancer types (the 22 cancer types that showed highly similar speckle protein gene PC1 weights to one taking the z-scores of speckle protein gene expression, calculated per cancer, and applying the following formula: sum((z-score Sig I speckle protein gene)*1/(number Sig I speckle protein genes))+sum((z-score Sig II speckle protein gene)*−1/(number Sig II speckle protein genes). In this manner a speckle score was assigned to samples so that it would be strongly positive for tumors with the strongest Signature I expression pattern and strongly negative for tumors with the strongest Signature II expression pattern. Speckle score was then used to separate samples into groups for Kaplan Meier and gene expression analysis between the two groups. With collected ccRCC samples and published drug response studies, speckle scores were calculated using the above formula. In drug-response data (related to FIG. 52), samples with positive speckle scores were considered speckle Signature I and samples with negative speckle scores were considered speckle Signature II. Then differences in drug responses were calculated using a Fisher's exact test (FIG. 53) or Kaplan Meier statistics (FIG. 54).

### Example 6: Nuclear Speckle Positioning Predicts Patient Prognosis in Neuroblastoma

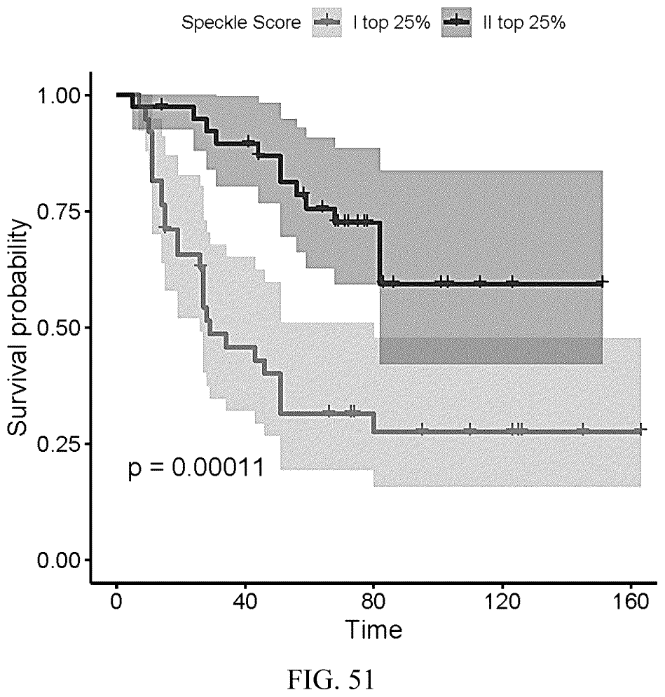

Without wishing to be bound by theory, it was hypothesized that the findings disclosed herein, where speckle score can be demonstrated to correlated with patient prognosis can be applied to different types of cancer. Having demonstrated a strong correlation in ccRCC, a studies was then carried out which used the speckle signature determining techniques disclosed herein to correlate survival and speckle score in neuroblastoma, a mostly pediatric cancer that develops in certain types of nervous tissues. RNA-seg and survival data from the TARGET 2018 study was analyzed and found to show that the speckle signature correlates with patient outcomes (FIG. 51), thus demonstrating the applicability of these methods to different kinds of cancer.

### Enumerated Embodiments

The following enumerated embodiments are provided, the numbering of which is not to be construed as designating levels of importance.

Embodiment 1 provides a polypeptide inhibitor of transcription factor/DNA-speckle association comprising a first polypeptide domain, a second polypeptide domain, and a third polypeptide domain, wherein:
a. the first polypeptide domain comprises a cell penetrating peptide;
b. the second polypeptide domain comprises a linker region; and
c. the third polypeptide domain comprises a DNA-speckle targeting motif.

Embodiment 2 provides the polypeptide of embodiment 1, wherein the cell penetrating peptide is selected from the group consisting of an HIV TAT peptide, a penetratin peptide, an R8 peptide, a transportan peptide, a cyclic R8 peptide, a cyclic TAT peptide, an HA-TAT peptide, and an xentry peptide.

Embodiment 3 provides the polypeptide of embodiment 2, wherein the cell penetrating peptide is an HIV TAT peptide.

Embodiment 4 provides the polypeptide of embodiment 3, wherein the HIV TAT peptide comprise an amino acid sequence of GRKKRRQRRRPQ (SEQ ID NO: 2603).

Embodiment 5 provides the polypeptide of embodiment 1, wherein the linker region comprises an amino acid sequence of GGSGGGSG (SEQ ID NO: 2604).

Embodiment 6 provides the polypeptide of embodiment 1, wherein the DNA-speckle targeting motif comprises a polypeptide sequence which is at least 62 amino acids.

Embodiment 7 provides the polypeptide of embodiment 6, wherein the polypeptide sequence comprises the pattern X1(30)-X2-P-X1(30), wherein
a. X1 is any amino acid; and
b. X2 is T, S, E, or D.

Embodiment 8 provides the polypeptide of embodiment 7, wherein the polypeptide sequence does not comprise four or more consecutive proline residues.

Embodiment 9 provides the polypeptide of embodiment 7, wherein the polypeptide sequence contains proline residues in a minimum of three of positions 16, 21, 36, 41, or 46.

Embodiment 10 provides the polypeptide of embodiment 7, wherein the polypeptide sequence comprises at least five negative or phosphorylatable amino acids.

Embodiment 11 provides the polypeptide of embodiment 10, wherein the negative or phosphorylatable amino acids are selected from the group consisting of D, E, T, and S.

Embodiment 12 provides the polypeptide of embodiment 7, wherein the polypeptide sequence comprises at least five small or hydrophobic amino acids.

Embodiment 13 provides the polypeptide of embodiment 12, wherein the small or hydrophobic amino acids are selected from the group consisting of A, M, V, F, L, and I.

Embodiment 14 provides the polypeptide of embodiment 7, wherein the polypeptide sequence comprises fewer than fifteen positively charged amino acids.

Embodiment 15 provides the polypeptide of embodiment 14, wherein the positively charged amino acids are selected from the group consisting of R, H, and K.

Embodiment 16 provides the polypeptide of embodiment 1, wherein the DNA-speckle targeting motif comprises an amino acid sequence set forth in any one of SEQ ID

Nos: 1-2602.

Embodiment 17 provides the polypeptide of embodiment 1, wherein the transcription factor is p53.

Embodiment 18 provides the polypeptide of embodiment 1, wherein the transcription factor is HIF2A.

Embodiment 19 provides a pharmaceutical composition comprising at least one polypeptide inhibitors of transcription factor/DNA-speckle association of any one of embodiments 1-18 and a pharmaceutically acceptable diluent or excipient.

Embodiment 20 provides a method for inhibiting transcription factor/DNA-speckle association in a cell, comprising contacting the cell with an effective amount of an inhibitor of transcription factor/DNA-speckle association, wherein the inhibitor is the polypeptide of any one of embodiments 1-18.

Embodiment 21 provides a method for inhibiting transcription factor/DNA-speckle association in a cell, comprising contacting the cell with an effective amount of an inhibitor of transcription factor/DNA-speckle association, wherein the inhibitor is a small molecule.

Embodiment 22 provides a method for inhibiting transcription factor/DNA-speckle association in a cell, comprising contacting the cell with an effective amount of an inhibitor of transcription factor/DNA-speckle association, wherein the inhibitor is a combination of a small molecule and the polypeptide of any one of embodiments 1-18.

Embodiment 23 provides a method of treating a DNA-speckle related cancer in a subject in need thereof, comprising administering to the subject an effective amount of the pharmaceutical composition of embodiment 19, thereby treating the cancer.

Embodiment 24 provides the method of embodiment 23, wherein the cancer is clear cell renal cell carcinoma (ccRCC).

Embodiment 25 provides the method of embodiment 23, wherein the cancer is selected from the group consisting of breast cancer, cervical squamous cell carcinoma, endocervical adenocarcinoma, colon adenocarcinoma, rectum adenocarcinoma, glioblastoma, head and neck squamous cell carcinoma, kidney renal papillary cell carcinoma, glioma, liver hepatocellular carcinoma, lung squamous cell carcinoma, lung adenocarcinoma, ovarian cancer, pheochromocytoma, paraganglioma, prostate adenocarcinoma, sarcoma, skin cutaneous melanoma, stomach adenocarcinoma, tenosynovial giant cell tumor, and thymoma.

Embodiment 26 provides a method of treating a DNA-speckle related cancer in a subject in need thereof, comprising administering to the subject an effective amount of the polypeptide of any one of embodiments 1-18, thereby treating the cancer.

Embodiment 27 provides the method of embodiment 27, wherein the cancer is clear cell renal cell carcinoma (ccRCC).

Embodiment 28 provides the method of embodiment 27, wherein the cancer is selected from the group consisting of breast cancer, cervical squamous cell carcinoma, endocervical adenocarcinoma, colon adenocarcinoma, rectum adenocarcinoma, glioblastoma, head and neck squamous cell carcinoma, kidney renal papillary cell carcinoma, glioma, liver hepatocellular carcinoma, lung squamous cell carcinoma, lung adenocarcinoma, ovarian cancer, pheochromocytoma, paraganglioma, prostate adenocarcinoma, sarcoma, skin cutaneous melanoma, stomach adenocarcinoma, tenosynovial giant cell tumor, and thymoma.

Embodiment 29 provides a method of generating peptide inhibitors of DNA speckle association, the method comprising:
a. screening a library of protein sequences for those comprising a DNA-speckle targeting motif comprising:
i. at least 62 contiguous amino acids;
ii. comprising the pattern X1(30)-X2-P-X1(30), wherein
iii. X1 is any amino acid; and
iv. X2 is T, S, E, or D;
v. does not comprise four or more consecutive proline residues;
vi. contains proline residues in a minimum of three of positions 16, 21, 36, 41, or 46;
vii. comprises at least five negative or phosphorylatable amino acids selected from the group consisting of D, E, T, and S;
viii. comprises at least five small or hydrophobic amino acids selected from the group consisting of A, M, V, F, L, and I; and
ix. comprises fewer than fifteen positively charged amino acids selected from the group consisting of R, H, and K;
b. identifying proteins comprising said motif sequence; and
c. generating peptides comprising said motif sequence.

Embodiment 30 provides the method of embodiment 29, wherein generating the peptide inhibitor further comprises adding a cell-permeability sequence to the DNA-speckle targeting motif sequence.

Embodiment 31 provides the method of embodiment 30, wherein the cell penetrating peptide is selected from the group consisting of an HIV TAT peptide, a penetratin peptide, an R8 peptide, a transportan peptide, a cyclic R8 peptide, a cyclic TAT peptide, an HA-TAT peptide, and an xentry peptide.

Embodiment 32 provides the method of embodiment 31, wherein the cell penetrating peptide is an HIV TAT peptide.

Embodiment 33 provides the method of embodiment 32, wherein the HIV TAT peptide comprise an amino acid sequence of GRKKRRQRRRPQ (SEQ ID NO: 2603).

Embodiment 34 provides the method of embodiment 29, wherein generating the peptide inhibitor further comprises adding a linker sequence between the cell-permeability sequence and the DNA-speckle targeting motif sequence.

Embodiment 35 provides the method of embodiment 34, wherein the linker region comprises an amino acid sequence of GGSGGGSG (SEQ ID NO: 2604).

Embodiment 36 provides a method of screening a tumor tissue to determine speckle signature score, comprising:
a. obtaining a specimen of tumor tissue;
b. isolating and purifying RNA from the specimen;
c. performing RNA-seq using the RNA to determine relative gene expression levels of Speckle signature genes;
d. determining the Z-score of each speckle signature gene;
e. for each speckle Signature I gene, divide its Z-score by the number of speckle protein genes in speckle Signature I, then take the sum of all these values for Signature I speckle protein genes;
f. for each speckle Signature II gene, divide its Z-score by the number of speckle protein genes in speckle Signature II, then take the sum of all these values for Signature II speckle protein genes; and
g. take the log(2) of the ratio of the result from step e to the result from step f thereby determining the speckle signature score of the specimen; wherein, samples with high positive values are strongly Signature I and samples with low negative values are strongly Signature II.

Embodiment 37 provides the method of embodiment 36, wherein the speckle signature comprises the genes FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, and EPC2.

Embodiment 38 provides the method of embodiment 36, wherein the genes comprising speckle Signature I are selected from the group consisting of VAX2, JDP2, PLEKHN1, HDAC5, C110RF49, SLC4A2, STYXL1, TMEM179B, TAB1, ZNF446, TBXA2R, UNC45A, PCBP1, PHLDB3, KTI12, AKAP17A, PRCC, ZNF821, SPINDOC, HSF4, DEXI, HEXIM2, EHMT2, VPS72, DDX39A, KIF22, DPCD, LHPP, CD2BP2, CDK11B, GTF2H4, DGKZ, SARNP, ALYREF, SLC2A4RG, TEPSIN, AKAP8L, PPIE, STK19, FIBP, C60RF226, H2AFX, EGFL8, PSMD13, CACTIN, EXOSC7, C120RF57, THAP4, TMEM259, THOC6, AP5Z1, PQBP1, RBM10, C1ORF35, C19ORF24, SART1, CDC34, FASTK, POMP, PRPF6, PRPF19, BRK1, UFC1, SNRPA1, ZCCHC17, SNRPB2, PCP2, SSH3, SETD1A, WDR90, THEM6, U2AF2, RBM14, MAST3, LIMK1, SF3B4, DDX39B, RTEL1, ZNF165, MAPK12, PSMD8, CDK5RAP1, PDZK1IP1, SETD4, CHTOP, CDK11A, SRSF4, TBX19, RTN2, CCDC32, CYSRT1, IQCK, MPP1, MAMSTR, ILRUN, DBNDD1, EPHB6, TCF15, C60RF52, CYGB, CCDC85C, PHYHD1, ITPKC, CDC25C, RMI2, SNRNP40, HISTIHIE, ZC3H18, SON, RBM27, TCF12, BCLAF1, ERBIN, SETD2, TCP11L2, EPC2, TRIP12, YLPM1, LMTK2, GPATCH8, DDX46, PRPF4B, TAB3, EPG5, RSBN1L, SF3B1, PUS7L, KCTD20, RBM26, BAZ2A, RBM41, RREB1, ZNF621, FAM160B1, CDK13, SDE2, DHX15, PRPF40A, CHIC1, SREK1, LIN52, BARD1, ZNF441, GNAQ, THRAP3, HBP1, SMC5, PPP4R3B, RBBP6, TTC26, COG6, ZC3H14, UBE3B, MRTFB, YTHDF3, UBE4A, CBLL1, API5, CMTR2, TBC1D12, WRN, KIAA1328, TMEM209, ZCCHC4, MAPK14, ZNF160, SLU7, ERCC8, FOXJ3, PCLO, RSRC1, ZC3H11A, BMP2K, RALGAPB, FBXL4, RTL6, RCAN3, FBXO34, ZBTB8A, CWF19L2, SRRM2, HELQ, FYTTD1, PPIG, ANKRD44, SOCS6, S100PBP, ZNF304, ZNF543, RBM25, EFCAB13, CPD, ARMCX5, POLI, ZNF551, MAML3, POLR3B, SFMBT2, DDX17, RNF169, KAT6A, DDX42, GPATCH2, CBFA2T2, E2F3, ZNF169, TAF5L, KIAA0100, PRKAA1, LHX4, RSRC2, CSRNP2, NCBP3, NCAPG2, SF3A1, DENNDlB, BRD2, PNISR, E2F7, LRRC8B, PACSIN2, PNN, KIAA0556, SAP130, CPSF6, MAP3K7, TADA2A, HP1BP3, ZNF217, BRD1, SRRM1, SRSF11, GLYR1, FAM227B, AAGAB, PLRG1, FCHSD2, MECOM, TMEM56, CDYL, ELOA, STK17A, RIOK1, ARHGAP42, R3HCC1L, COPS4, BORCS7, THOC1, CIR1, PYROXD1, ARHGAP18, NSL1, WTAP, ZNHIT6, BCAS2, HAUS6, MORF4L1, SMC4, MBD4, PRPF18, CWC22, UBAP2L, SMURF2, KDM6B, PRKAA2, LIFR, RBM8A, SNURF, DAZAP2, FAM120C, WDR17, ZDHHC15, GTF2H2C, SRGAP1, ZSWIM5, RAF1, ZNF286B, ZNF528, ZNF572, ZNF527, XYLB, FNBP4, PRPF4, SIPA1L3, ZNF382, RFXAP, RBM39, CWC25, ZIM2, ANXA9, MFSD11, BPNT1, GPN3, MAPT, PPP1R16B, ZNF250, RAD52, ZNF786, GNB5, MNS1, TARBP1, RBM6, PRKN, ZCWPW2, MAMDC2, IPCEF1, NFATC4, LPAR1, VXN, FAM107A, IL16, USP22, RNF112, CRY2, PLAGI, IQUB, PPP1R8, BNIP3L or any combination thereof.

Embodiment 39 provides the method of embodiment 36, wherein the genes comprising speckle Signature II are selected from the group consisting of SON, RBM27, TCF12, BCLAF1, ERBIN, SETD2, TCP11L2, EPC2, TRIP12, YLPM1, LMTK2, GPATCH8, DDX46, PRPF4B, TAB3, EPG5, RSBN1L, SF3B1, PUS7L, KCTD20, RBM26, BAZ2A, RBM41, RREB1, ZNF621, FAM160B1, CDK13, SDE2, DHX15, PRPF40A, CHIC1, SREK1, LIN52, BARD1, ZNF441, GNAQ, THRAP3, HBP1, SMC5, PPP4R3B, RBBP6, TTC26, COG6, ZC3H14, UBE3B, MRTFB, YTHDF3, UBE4A, CBLL1, API5, CMTR2, TBC1D12, WRN, KIAA1328, TMEM209, ZCCHC4, MAPK14, ZNF160, SLU7, ERCC8, FOXJ3, PCLO, RSRC1, ZC3H11A, BMP2K, RALGAPB, FBXL4, RTL6, RCAN3, FBXO34, ZBTB8A, CWF19L2, SRRM2, HELQ, FYTTD1, PPIG, ANKRD44, SOCS6, S100PBP, ZNF304, ZNF543, RBM25, EFCAB13, CPD, ARMCX5, POLI, ZNF551, MAML3, POLR3B, SFMBT2, DDX17, RNF169, KAT6A, DDX42, GPATCH2, CBFA2T2, E2F3, ZNF169, TAF5L, KIAA0100, PRKAA1, LHX4, RSRC2, CSRNP2, NCBP3, NCAPG2, SF3A1, DENND1B, BRD2, PNISR, E2F7, LRRC8B, PACSIN2, PNN, KIAA0556, SAP130, CPSF6, MAP3K7, TADA2A, HP1BP3, ZNF217, BRD1, SRRM1, SRSF11, GLYR1, FAM227B, AAGAB, PLRG1, FCHSD2, MECOM, TMEM56, CDYL, ELOA, STK17A, RIOK1, ARHGAP42, R3HCC1L, COPS4, BORCS7, THOC1, CIR1, PYROXD1, ARHGAP18, NSL1, WTAP, ZNHIT6, BCAS2, HAUS6, MORF4L1, SMC4, MBD4, PRPF18, CWC22, UBAP2L, SMURF2, KDM6B, PRKAA2, LIFR, RBM8A, SNURF, DAZAP2, FAM120C, WDR17, ZDHHC15, GTF2H2C, SRGAP1, ZSWIM5, RAF1, ZNF286B, ZNF528, ZNF572, ZNF527, XYLB, FNBP4, PRPF4, SIPA1L3, ZNF382, RFXAP, RBM39, CWC25, ZIM2, ANXA9, MFSD11, BPNT1, GPN3, MAPT, PPP1R16B, ZNF250, RAD52, ZNF786, GNB5, MNS1, TARBP1, RBM6, PRKN, ZCWPW2, MAMDC2, IPCEF1, NFATC4, LPAR1, VXN, FAM107A, IL16, USP22, RNF112, CRY2, PLAGI, IQUB, PPP1R8, BNIP3L, VAX2, JDP2, PLEKHN1, HDAC5, C11ORF49, SLC4A2, STYXL1, TMEM179B, TAB1, ZNF446, TBXA2R, UNC45A, PCBP1, PHLDB3, KTI12, AKAP17A, PRCC, ZNF821, SPINDOC, HSF4, DEXI, HEXIM2, EHMT2, VPS72, DDX39A, KIF22, DPCD, LHPP, CD2BP2, CDK11B, GTF2H4, DGKZ, SARNP, ALYREF, SLC2A4RG, TEPSIN, AKAP8L, PPIE, STK19, FIBP, C60RF226, H2AFX, EGFL8, PSMD13, CACTIN, EXOSC7, C120RF57, THAP4, TMEM259, THOC6, AP5Z1, PQBP1, RBM10, C1ORF35, C19ORF24, SART1, CDC34, FASTK, POMP, PRPF6, PRPF19, BRK1, UFC1, SNRPA1, ZCCHC17, SNRPB2, PCP2, SSH3, SETD1A, WDR90, THEM6, U2AF2, RBM14, MAST3, LIMK1, SF3B4, DDX39B, RTEL1, ZNF165, MAPK12, PSMD8, CDK5RAP1, PDZK1IP1, SETD4, CHTOP, CDK11A, SRSF4, TBX19, RTN2, CCDC32, CYSRT1, IQCK, MPP1, MAMSTR, ILRUN, DBNDD1, EPHB6, TCF15, C60RF52, CYGB, CCDC85C, PHYHD1, ITPKC, CDC25C, RMI2, SNRNP40, HISTIHIE, ZC3H18.

Embodiment 40 provides a method of treating a Speckle signature associated cancer in a subject in need thereof, comprising:
a. obtaining a specimen of tumor tissue;
b. isolating and purifying RNA from the specimen;
c. performing RNA-seq using the RNA to determine the speckle signature of the tumor tissue; and
d. administering an effective amount of an inhibitor of expression for at least one speckle signature gene, thereby treating the cancer;

Embodiment 41 provides the method of embodiment 40, further comprising determining the nuclear localization profile of at least one speckle signature gene.

Embodiment 42 provides the method of embodiment 41, wherein a radial nuclear localization profile correlates with worse prognosis.

Embodiment 43 provides the method of embodiment 41, wherein the at least one inhibited speckle gene is associated with speckle Signature I.

Embodiment 44 provides the method of embodiment 42, wherein the inhibition of at least one gene associated with Speckle Signature I shifts the Speckle signature of the tumor tissue to Speckle Signature II.

Embodiment 45 provides the method of embodiment 41, wherein the at least one inhibited Speckle gene is associated with Speckle Signature II.

Embodiment 46 provides the method of embodiment 44, wherein the inhibition of at least one gene associated with Speckle Signature II shifts the Speckle signature of the tumor tissue to Speckle Signature I.

Embodiment 47 provides the method of any one of embodiments 41-45, wherein shifting the Speckle signature of the tumor tissue improves prognosis.

Embodiment 48 provides the method of embodiment 41, wherein the cancer is selected from the group consisting of clear cell renal cell carcinoma, KMT2D wild type melanoma, TTN wild type lung adenocarcinoma, BRAF wild type thyroid cancer, and PIK3R1 mutant endometrial cancer.

Embodiment 49 provides the method of embodiment 41, wherein the inhibitor of Speckle signature gene expression is selected from the group consisting of an inhibitory RNA, a small molecule, a PROTAC, a CRISPR/Cas9 system, and any combination thereof.

Embodiment 50 provides the method of embodiment 48, wherein the inhibitory RNA is selected from the group consisting of an siRNA, and an shRNA or any combination thereof.

Embodiment 51 provides the method of embodiment 41, wherein the Speckle signature gene is SART1.

Embodiment 52 provides the method of embodiment 41, wherein the speckle signature gene is HBP1.

Embodiment 53 provides the method of embodiment 41, wherein the speckle signature gene is COPS4

Embodiment 54 provides the method of embodiment 41, wherein the speckle signature is determined by immunofluorescence of FFPE tumor samples.

Embodiment 55 provides the method of embodiment 41, wherein the speckle signature is determined by RNA or protein analysis of a subset of speckle protein genes comprising FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, EPC2, or any combination thereof.

Embodiment 56 provides a method of determining the prognosis of a speckle-related cancer in a subject in need thereof, comprising:
d. obtaining a specimen of cancer tissue;
e. preparing the tissue specimen such that nuclear localization of at least one speckle-related protein can be visualized and quantified; and
f. determining the nuclear localization profile of at least one speckle-related protein in the tissue, thereby indicating the severity of the speckle-related cancer;
wherein radial positioning speckle-related protein expression indicates a worse prognosis.

Embodiment 57 provides the method of embodiment 56, wherein the at least one speckle-related protein is selected from the group consisting of FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, EPC2, or any combination thereof.

Embodiment 58 provides the method of embodiment 56, wherein the at least one speckle-related protein is SON.

Embodiment 59 provides the method of embodiment 56, wherein the visualization and quantification of the speckle protein localization comprises immunofluorescence microscopy.

Embodiment 60 provides the method of embodiment 56, wherein the cancer is selected from the group consisting of clear cell renal cell carcinoma, KMT2D wild type melanoma, TTN wild type lung adenocarcinoma, BRAF wild type thyroid cancer, and PIK3R1 mutant endometrial cancer.

Embodiment 61 provides a method of treating a speckle-related cancer in a subject in need thereof, comprising:
c. performing RNA-seq using RNA purified from a tumor specimen from the subject to determine the speckle signature of the tumor tissue; and
d. administering an effective amount of an anticancer therapeutic, thereby treating the cancer;
wherein, the sensitivity of the tumor to the anticancer therapeutic correlates with the speckle signature of the tumor tissue.

Embodiment 62 provides the method of embodiment 61, further comprising determining the nuclear localization profile of nuclear speckles.

Embodiment 63 provides the method of embodiment 61, wherein the speckle signature is associated with speckle signature I.

Embodiment 64 provides the method of embodiment 61, wherein the speckle signature is associated with speckle Signature II.

Embodiment 65 provides the method of embodiments 61, wherein choosing a speckle signature correlated treatment strategy improves treatment prognosis.

Embodiment 66 provides the method of embodiment 61, wherein the cancer is selected from the group consisting of clear cell renal cell carcinoma, neuroblastoma, KMT2D wild type melanoma, TTN wild type lung adenocarcinoma, BRAF wild type thyroid cancer, and PIK3R1 mutant endometrial cancer.

Embodiment 67 provides the method of embodiment 61, wherein the cancer is clear cell renal cell carcinoma.

Embodiment 68 provides the method of embodiment 61, wherein the anticancer therapeutic is selected from the group consisting of a biologic, a small molecule, an immunotherapy, and any combination thereof.

Embodiment 69 provides the method of embodiment 67, wherein the immunotherapy is an immune checkpoint inhibitor.

Embodiment 70 provides the method of embodiment 68, wherein the immune checkpoint inhibitor is an inhibitor of PD-1.

Embodiment 71 provides the method of embodiment 69, wherein the PD-1 inhibitor is nivolumab.

Embodiment 72 provides the method of embodiment 61, wherein the anticancer therapeutic is an inhibitor of HIF-2a.

Embodiment 73 provides the method of embodiment 72, wherein the inhibitor of HIF-2a is PT2399.

Embodiment 74 provides the method of embodiment 62, wherein the speckle signature is determined by the nuclear localization profile of nuclear speckles.

Embodiment 75 provides the method of embodiment 74, wherein the nuclear localization profile is determined by immunofluorescence of FFPE tumor samples.

Embodiment 76 provides the method of embodiment 61, wherein the speckle signature is determined by RNA or protein analysis of a subset of speckle protein genes comprising FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, EPC2, or any combination thereof.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to Ie all such embodiments and equivalent variations.

What is claimed:

1. A polypeptide inhibitor of transcription factor/DNA-speckle association comprising a first polypeptide domain, a second polypeptide domain, and a third polypeptide domain, wherein:
a. the first polypeptide domain comprises a cell penetrating peptide;
b. the second polypeptide domain comprises a linker region; and
c. the third polypeptide domain comprises a DNA-speckle targeting motif.

2. The polypeptide of claim 1, wherein the cell penetrating peptide is selected from the group consisting of an HIV TAT peptide, a penetratin peptide, an R8 peptide, a transportan peptide, a cyclic R8 peptide, a cyclic TAT peptide, an HA-TAT peptide, and an xentry peptide.

3. The polypeptide of claim 2, wherein the cell penetrating peptide is an HIV TAT peptide.

4. The polypeptide of claim 3, wherein the HIV TAT peptide comprise an amino acid sequence of GRKKRRQRRRPQ (SEQ ID NO: 2603).

5. The polypeptide of claim 1, wherein the linker region comprises an amino acid sequence of GGSGGGSG (SEQ ID NO: 2604).

6. The polypeptide of claim 1, wherein the DNA-speckle targeting motif comprises a polypeptide sequence which is at least 62 amino acids.

7. The polypeptide of claim 6, wherein the polypeptide sequence comprises the pattern X₁(30)-X₂-P-X₁(30), wherein
a. X₁ is any amino acid; and
b. X₂ is T, S, E, or D.

8. The polypeptide of claim 7, wherein the polypeptide sequence does not comprise four or more consecutive proline residues.

9. The polypeptide of claim 7, wherein the polypeptide sequence contains proline residues in a minimum of three of positions 16, 21, 36, 41, or 46.

10. The polypeptide of claim 7, wherein the polypeptide sequence comprises at least five negative or phosphorylatable amino acids.

11. The polypeptide of claim 10, wherein the negative or phosphorylatable amino acids are selected from the group consisting of D, E, T, and S.

12. The polypeptide of claim 7, wherein the polypeptide sequence comprises at least five small or hydrophobic amino acids.

13. The polypeptide of claim 12, wherein the small or hydrophobic amino acids are selected from the group consisting of A, M, V, F, L, and I.

14. The polypeptide of claim 7, wherein the polypeptide sequence comprises fewer than fifteen positively charged amino acids.

15. The polypeptide of claim 14, wherein the positively charged amino acids are selected from the group consisting of R, H, and K.

16. The polypeptide of claim 1, wherein the DNA-speckle targeting motif comprises an amino acid sequence set forth in any one of SEQ ID Nos: 1-2602.

17. The polypeptide of claim 1, wherein the transcription factor is p53.

18. The polypeptide of claim 1, wherein the transcription factor is HIF2A.

19. A pharmaceutical composition comprising at least one polypeptide inhibitors of transcription factor/DNA-speckle association of claim 1 and a pharmaceutically acceptable diluent or excipient.

20. A method for inhibiting transcription factor/DNA-speckle association in a cell, comprising contacting the cell with an effective amount of an inhibitor of transcription factor/DNA-speckle association, wherein the inhibitor is the polypeptide of claim 1.

21. A method for inhibiting transcription factor/DNA-speckle association in a cell, comprising contacting the cell with an effective amount of an inhibitor of transcription factor/DNA-speckle association, wherein the inhibitor is a small molecule.

22. A method for inhibiting transcription factor/DNA-speckle association in a cell, comprising contacting the cell with an effective amount of an inhibitor of transcription factor/DNA-speckle association, wherein the inhibitor is a combination of a small molecule and the polypeptide of claim 1.

23. A method of treating a DNA-speckle related cancer in a subject in need thereof, comprising administering to the subject an effective amount of the pharmaceutical composition of claim 19, thereby treating the cancer.

24. The method of claim 23, wherein the cancer is clear cell renal cell carcinoma (ccRCC).

25. The method of claim 23, wherein the cancer is selected from the group consisting of breast cancer, cervical squamous cell carcinoma, endocervical adenocarcinoma, colon adenocarcinoma, rectum adenocarcinoma, glioblastoma, head and neck squamous cell carcinoma, kidney renal papillary cell carcinoma, glioma, liver hepatocellular carcinoma, lung squamous cell carcinoma, lung adenocarcinoma, ovarian cancer, pheochromocytoma, paraganglioma, prostate adenocarcinoma, sarcoma, skin cutaneous melanoma, stomach adenocarcinoma, tenosynovial giant cell tumor, and thymoma.

26. A method of treating a DNA-speckle related cancer in a subject in need thereof, comprising administering to the subject an effective amount of the polypeptide of claim 1, thereby treating the cancer.

27. The method of claim 26, wherein the cancer is clear cell renal cell carcinoma (ccRCC).

28. The method of claim 26, wherein the cancer is selected from the group consisting of breast cancer, cervical squamous cell carcinoma, endocervical adenocarcinoma, colon adenocarcinoma, rectum adenocarcinoma, glioblastoma, head and neck squamous cell carcinoma, kidney renal papillary cell carcinoma, glioma, liver hepatocellular carcinoma, lung squamous cell carcinoma, lung adenocarcinoma, ovarian cancer, pheochromocytoma, paraganglioma, prostate adenocarcinoma, sarcoma, skin cutaneous melanoma, stomach adenocarcinoma, tenosynovial giant cell tumor, and thymoma.

29. A method of generating peptide inhibitors of DNA speckle association, the method comprising:
a. screening a library of protein sequences for those comprising a DNA-speckle targeting motif comprising:
i. at least 62 contiguous amino acids;
ii. comprising the pattern X₁(30)-X₂-P-X₁(30), wherein
X₁ is any amino acid; and
X₂ is T, S, E, or D;
iii. does not comprise four or more consecutive proline residues;
iv. contains proline residues in a minimum of three of positions 16, 21, 36, 41, or 46;
v. comprises at least five negative or phosphorylatable amino acids selected from the group consisting of D, E, T, and S;
vi. comprises at least five small or hydrophobic amino acids selected from the group consisting of A, M, V, F, L, and I; and
vii. comprises fewer than fifteen positively charged amino acids selected from the group consisting of R, H, and K;
b. identifying proteins comprising said motif sequence; and
c. generating peptides comprising said motif sequence.

30. The method of claim 29, wherein generating the peptide inhibitor further comprises adding a cell-permeability sequence to the DNA-speckle targeting motif sequence.

31. The method of claim 30, wherein the cell penetrating peptide is selected from the group consisting of an HIV TAT peptide, a penetratin peptide, an R8 peptide, a transportan peptide, a cyclic R8 peptide, a cyclic TAT peptide, an HA-TAT peptide, and an xentry peptide.

32. The method of claim 31, wherein the cell penetrating peptide is an HIV TAT peptide.

33. The method of claim 32, wherein the HIV TAT peptide comprise an amino acid sequence of GRKKRRQRRRPQ (SEQ ID NO: 2603).

34. The method of claim 29, wherein generating the peptide inhibitor further comprises adding a linker sequence between the cell-permeability sequence and the DNA-speckle targeting motif sequence.

35. The method of claim 34, wherein the linker region comprises an amino acid sequence of GGSGGGSG (SEQ ID NO: 2604).

36. A method of screening a tumor tissue to determine speckle signature score, comprising:
a. obtaining a specimen of tumor tissue;
b. isolating and purifying RNA from the specimen;
c. performing RNA-seq using the RNA to determine relative gene expression levels of speckle signature genes;
d. determining the Z-score of each speckle signature gene;
e. for each speckle Signature I gene, divide its Z-score by the number of speckle protein genes in speckle signature I, then take the sum of all these values for Signature I speckle protein genes;
f. for each speckle Signature II gene, divide its Z-score by the number of speckle protein genes in speckle signature II, then take the sum of all these values for Signature II speckle protein genes; and
g. take the log(2) of the ratio of the result from step e to the result from step f thereby determining the speckle signature score of the specimen;
wherein, samples with high positive values are strongly Signature I and samples with low negative values are strongly Signature II.

37. The method of claim 36, wherein the speckle signature comprises the genes FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, and EPC2.

38. The method of claim 36, wherein the genes comprising speckle Signature I are selected from the group consisting of VAX2, JDP2, PLEKHN1, HDAC5, C110RF49, SLC4A2, STYXL1, TMEM179B, TAB1, ZNF446, TBXA2R, UNC45A, PCBP1, PHLDB3, KTI12, AKAP17A, PRCC, ZNF821, SPINDOC, HSF4, DEXI, HEXIM2, EHMT2, VPS72, DDX39A, KIF22, DPCD, LHPP, CD2BP2, CDK11B, GTF2H4, DGKZ, SARNP, ALYREF, SLC2A4RG, TEPSIN, AKAP8L, PPIE, STK19, FIBP, C60RF226, H2AFX, EGFL8, PSMD13, CACTIN, EXOSC7, C120RF57, THAP4, TMEM259, THOC6, AP5Z1, PQBP1, RBM10, C10RF35, C19ORF24, SART1, CDC34, FASTK, POMP, PRPF6, PRPF19, BRK1, UFC1, SNRPA1, ZCCHC17, SNRPB2, PCP2, SSH3, SETD1A, WDR90, THEM6, U2AF2, RBM14, MAST3, LIMK1, SF3B4, DDX39B, RTEL1, ZNF165, MAPK12, PSMD8, CDK5RAP1, PDZK1IP1, SETD4, CHTOP, CDK11A, SRSF4, TBX19, RTN2, CCDC32, CYSRT1, IQCK, MPP1, MAMSTR, ILRUN, DBNDD1, EPHB6, TCF15, C60RF52, CYGB, CCDC85C, PHYHD1, ITPKC, CDC25C, RMI2, SNRNP40, HISTIHIE, ZC3H18, SON, RBM27, TCF12, BCLAF1, ERBIN, SETD2, TCP11L2, EPC2, TRIP12, YLPM1, LMTK2, GPATCH8, DDX46, PRPF4B, TAB3, EPG5, RSBN1L, SF3B1, PUS7L, KCTD20, RBM26, BAZ2A, RBM41, RREB1, ZNF621, FAM160B1, CDK13, SDE2, DHX15, PRPF40A, CHIC1, SREK1, LIN52, BARD1, ZNF441, GNAQ, THRAP3, HBP1, SMC5, PPP4R3B, RBBP6, TTC26, COG6, ZC3H14, UBE3B, MRTFB, YTHDF3, UBE4A, CBLL1, API5, CMTR2, TBC1D12, WRN, KIAA1328, TMEM209, ZCCHC4, MAPK14, ZNF160, SLU7, ERCC8, FOXJ3, PCLO, RSRC1, ZC3H11A, BMP2K, RALGAPB, FBXL4, RTL6, RCAN3, FBXO34, ZBTB8A, CWF19L2, SRRM2, HELQ, FYTTD1, PPIG, ANKRD44, SOCS6, S100PBP, ZNF304, ZNF543, RBM25, EFCAB13, CPD, ARMCX5, POLI, ZNF551, MAML3, POLR3B, SFMBT2, DDX17, RNF169, KAT6A, DDX42, GPATCH2, CBFA2T2, E2F3, ZNF169, TAF5L, KIAA0100, PRKAA1, LHX4, RSRC2, CSRNP2, NCBP3, NCAPG2, SF3A1, DENND1B, BRD2, PNISR, E2F7, LRRC8B, PACSIN2, PNN, KIAA0556, SAP130, CPSF6, MAP3K7, TADA2A, HP1BP3, ZNF217, BRD1, SRRM1, SRSF11, GLYR1, FAM227B, AAGAB, PLRG1, FCHSD2, MECOM, TMEM56, CDYL, ELOA, STK17A, RIOK1, ARHGAP42, R3HCC1L, COPS4, BORCS7, THOC1, CIR1, PYROXD1, ARHGAP18, NSL1, WTAP, ZNHIT6, BCAS2, HAUS6, MORF4L1, SMC4, MBD4, PRPF18, CWC22, UBAP2L, SMURF2, KDM6B, PRKAA2, LIFR, RBM8A, SNURF, DAZAP2, FAM120C, WDR17, ZDHHC15, GTF2H2C, SRGAP1, ZSWIM5, RAF1, ZNF286B, ZNF528, ZNF572, ZNF527, XYLB, FNBP4, PRPF4, SIPA1L3, ZNF382, RFXAP, RBM39, CWC25, ZIM2, ANXA9, MFSD11, BPNT1, GPN3, MAPT, PPP1R16B, ZNF250, RAD52, ZNF786, GNB5, MNS1, TARBP1, RBM6, PRKN, ZCWPW2, MAMDC2, IPCEF1, NFATC4, LPAR1, VXN, FAM107A, IL16, USP22, RNF112, CRY2, PLAGI, IQUB, PPP1R8, BNIP3L or any combination thereof.

39. The method of claim 36, wherein the genes comprising speckle Signature II are selected from the group consisting of SON, RBM27, TCF12, BCLAF1, ERBIN, SETD2, TCP11L2, EPC2, TRIP12, YLPM1, LMTK2, GPATCH8, DDX46, PRPF4B, TAB3, EPG5, RSBN1L, SF3B1, PUS7L, KCTD20, RBM26, BAZ2A, RBM41, RREB1, ZNF621, FAM160B1, CDK13, SDE2, DHX15, PRPF40A, CHIC1, SREK1, LIN52, BARD1, ZNF441, GNAQ, THRAP3, HBP1, SMC5, PPP4R3B, RBBP6, TTC26, COG6, ZC3H14, UBE3B, MRTFB, YTHDF3, UBE4A, CBLL1, API5, CMTR2, TBC1D12, WRN, KIAA1328, TMEM209, ZCCHC4, MAPK14, ZNF160, SLU7, ERCC8, FOXJ3, PCLO, RSRC1, ZC3H11A, BMP2K, RALGAPB, FBXL4, RTL6, RCAN3, FBXO34, ZBTB8A, CWF19L2, SRRM2, HELQ, FYTTD1, PPIG, ANKRD44, SOCS6, S100PBP, ZNF304, ZNF543, RBM25, EFCAB13, CPD, ARMCX5, POLI, ZNF551, MAML3, POLR3B, SFMBT2, DDX17, RNF169, KAT6A, DDX42, GPATCH2, CBFA2T2, E2F3, ZNF169, TAF5L, KIAA0100, PRKAA1, LHX4, RSRC2, CSRNP2, NCBP3, NCAPG2, SF3A1, DENND1B, BRD2, PNISR, E2F7, LRRC8B, PACSIN2, PNN, KIAA0556, SAP130, CPSF6, MAP3K7, TADA2A, HP1BP3, ZNF217, BRD1, SRRM1, SRSF11, GLYR1, FAM227B, AAGAB, PLRG1, FCHSD2, MECOM, TMEM56, CDYL, ELOA, STK17A, RIOK1, ARHGAP42, R3HCC1L, COPS4, BORCS7, THOC1, CIR1, PYROXD1, ARHGAP18, NSL1, WTAP, ZNHIT6, BCAS2, HAUS6, MORF4L1, SMC4, MBD4, PRPF18, CWC22, UBAP2L, SMURF2, KDM6B, PRKAA2, LIFR, RBM8A, SNURF, DAZAP2, FAM120C, WDR17, ZDHHC15, GTF2H2C, SRGAP1, ZSWIM5, RAF1, ZNF286B, ZNF528, ZNF572, ZNF527, XYLB, FNBP4, PRPF4, SIPA1L3, ZNF382, RFXAP, RBM39, CWC25, ZIM2, ANXA9, MFSD11, BPNT1, GPN3, MAPT, PPP1R16B, ZNF250, RAD52, ZNF786, GNB5, MNS1, TARBP1, RBM6, PRKN, ZCWPW2, MAMDC2, IPCEF1, NFATC4, LPAR1, VXN, FAM107A, IL16, USP22, RNF112, CRY2, PLAGI, IQUB, PPP1R8, BNIP3L, VAX2, JDP2, PLEKHN1, HDAC5, C11ORF49, SLC4A2, STYXL1, TMEM179B, TAB1, ZNF446, TBXA2R, UNC45A, PCBP1, PHLDB3, KTI12, AKAP17A, PRCC, ZNF821, SPINDOC, HSF4, DEXI, HEXIM2, EHMT2, VPS72, DDX39A, KIF22, DPCD, LHPP, CD2BP2, CDK11B, GTF2H4, DGKZ, SARNP, ALYREF, SLC2A4RG, TEPSIN, AKAP8L, PPIE, STK19, FIBP, C60RF226, H2AFX, EGFL8, PSMD13, CACTIN, EXOSC7, C120RF57, THAP4, TMEM259, THOC6, AP5Z1, PQBP1, RBM10, C1ORF35, C19ORF24, SART1, CDC34, FASTK, POMP, PRPF6, PRPF19, BRK1, UFC1, SNRPA1, ZCCHC17, SNRPB2, PCP2, SSH3, SETD1A, WDR90, THEM6, U2AF2, RBM14, MAST3, LIMK1, SF3B4, DDX39B, RTEL1, ZNF165, MAPK12, PSMD8, CDK5RAP1, PDZK1IP1, SETD4, CHTOP, CDK11A, SRSF4, TBX19, RTN2, CCDC32, CYSRT1, IQCK, MPP1, MAMSTR, ILRUN, DBNDD1, EPHB6, TCF15, C60RF52, CYGB, CCDC85C, PHYHD1, ITPKC, CDC25C, RMI2, SNRNP40, HISTIHIE, ZC3H18.

40. A method of treating a speckle signature associated cancer in a subject in need thereof, comprising:
a. obtaining a specimen of tumor tissue;
b. isolating and purifying RNA from the specimen;
c. performing RNA-seq using the RNA to determine the speckle signature of the tumor tissue; and
d. administering an effective amount of an inhibitor of expression for at least one speckle signature gene, thereby treating the cancer.

41. The method of claim 40, further comprising determining the nuclear localization profile of at least one speckle signature gene.

42. The method of claim 41, wherein a radial nuclear localization profile correlates with worse prognosis.

43. The method of claim 40, wherein the at least one inhibited speckle protein gene is associated with speckle signature I.

44. The method of claim 43, wherein the inhibition of at least one gene associated with speckle Signature I shifts the speckle signature of the tumor tissue to speckle Signature II.

45. The method of claim 40, wherein the at least one inhibited speckle protein gene is associated with speckle Signature II.

46. The method of claim 45, wherein the inhibition of at least one gene associated with speckle Signature II shifts the speckle signature of the tumor tissue to speckle Signature I.

47. The method of claim 40, wherein inhibiting the expression of the speckle signature gene shifts the speckle signature of the tumor tissue and improves prognosis.

48. The method of claim 40, wherein the cancer is selected from the group consisting of clear cell renal cell carcinoma, KMT2D wild type melanoma, TTN wild type lung adenocarcinoma, BRAF wild type thyroid cancer, and PIK3R1 mutant endometrial cancer.

49. The method of claim 40, wherein the inhibitor of speckle signature gene expression is selected from the group consisting of an inhibitory RNA, a small molecule, a PROTAC, a CRISPR/Cas9 system, and any combination thereof.

50. The method of claim 47, wherein the inhibitory RNA is selected from the group consisting of an siRNA, and an shRNA or any combination thereof.

51. The method of claim 40, wherein the speckle signature gene is SART1.

52. The method of claim 40, wherein the speckle signature gene is HBP1.

53. The method of claim 40, wherein the speckle signature gene is COPS4.

54. The method of claim 40, wherein the speckle signature is determined by immunofluorescence of FFPE tumor samples.

55. The method of claim 40, wherein the speckle signature is determined by RNA or protein analysis of a subset of speckle protein genes comprising FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, EPC2, or any combination thereof.

56. A method of determining the prognosis of a speckle-related cancer in a subject in need thereof, comprising:
a. obtaining a specimen of cancer tissue;
b. preparing the tissue specimen such that nuclear localization of at least one speckle-related protein can be visualized and quantified; and
c. determining the nuclear localization profile of at least one speckle-related protein in the tissue, thereby indicating the severity of the speckle-related cancer;
wherein radial positioning speckle-related protein expression indicates a worse prognosis.

57. The method of claim 56, wherein the at least one speckle-related protein is selected from the group consisting of FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, EPC2, or any combination thereof.

58. The method of claim 56, wherein the at least one speckle-related protein is SON.

59. The method of claim 56, wherein the visualization and quantification of the speckle protein localization comprises immunofluorescence microscopy.

60. The method of claim 56, wherein the cancer is selected from the group consisting of clear cell renal cell carcinoma, KMT2D wild type melanoma, TTN wild type lung adenocarcinoma, BRAF wild type thyroid cancer, and PIK3R1 mutant endometrial cancer.

61. A method of treating a speckle-related cancer in a subject in need thereof, comprising:
a. performing RNA-seq using RNA purified from a tumor specimen from the subject to determine the speckle signature of the tumor tissue; and
b. administering an effective amount of an anticancer therapeutic, thereby treating the cancer;
wherein, the sensitivity of the tumor to the anticancer therapeutic correlates with the speckle signature of the tumor tissue.

62. The method of claim 61, further comprising determining the nuclear localization profile of nuclear speckles.

63. The method of claim 61, wherein the speckle signature is associated with speckle signature I.

64. The method of claim 61, wherein the speckle signature is associated with speckle Signature II.

65. The method of claim 61, wherein choosing a speckle signature correlated treatment strategy improves treatment prognosis.

66. The method of claim 61, wherein the cancer is selected from the group consisting of clear cell renal cell carcinoma, neuroblastoma, KMT2D wild type melanoma, TTN wild type lung adenocarcinoma, BRAF wild type thyroid cancer, and PIK3R1 mutant endometrial cancer.

67. The method of claim 61, wherein the cancer is clear cell renal cell carcinoma.

68. The method of claim 61, wherein the anticancer therapeutic is selected from the group consisting of an a biologic, a small molecule, an immunotherapy, and any combination thereof.

69. The method of claim 67, wherein the immunotherapy is an immune checkpoint inhibitor.

70. The method of claim 68, wherein the immune checkpoint inhibitor is an inhibitor of PD-1.

71. The method of claim 69, wherein the PD-1 inhibitor is nivolumab.

72. The method of claim 61, wherein the anticancer therapeutic is an inhibitor of HIF-2a.

73. The method of claim 72, wherein the inhibitor of HIF-2a is PT2399.

74. The method of claim 61, wherein the speckle signature is determined by the nuclear localization profile of nuclear speckles.

75. The method of claim 74, wherein the nuclear localization profile is determined by immunofluorescence of FFPE tumor samples.

76. The method of claim 61, wherein the speckle signature is determined by RNA or protein analysis of a subset of speckle protein genes comprising FIBP, PQBP1, SART1, THRAP4, FASTK, C19ORF24, CDC34, FBXL4, WRN, RNF169, TRIP12, SON, RBM27, BCLAF1, PRPF4B, SETD2, RBM26, EPC2, or any combination thereof.
